SEQUENCE LISTING (541 sequences)

SEQ ID NO: 1 - protein, 5 aa, synthetic construct
GSDMS

SEQ ID NO: 2 - protein, 16 aa, synthetic construct
YITSGGTTYYPDSVKG

SEQ ID NO: 3 - protein, 12 aa, synthetic construct
ARLTDSHYVEDA

SEQ ID NO: 4 - protein, 5 aa, synthetic construct
SADMS

SEQ ID NO: 5 - protein, 17 aa, synthetic construct
YINADGSKTYYPDSVKG

SEQ ID NO: 6 - protein, 12 aa, synthetic construct
AKLTDTHYVEDY

SEQ ID NO: 7 - protein, 5 aa, synthetic construct
NYDMS

SEQ ID NO: 8 - protein, 17 aa, synthetic construct
YINAGGSNTDYPDSVKG

SEQ ID NO: 9 - protein, 12 aa, synthetic construct
AKLTDTHYVEDY

SEQ ID NO: 10 - protein, 5 aa, synthetic construct
GSDMS

SEQ ID NO: 11 - protein, 16 aa, synthetic construct
YITGSGTTYYPDSVKG

SEQ ID NO: 12 - protein, 12 aa, synthetic construct
ARLTDSHYVEDA

SEQ ID NO: 13 - protein, 5 aa, synthetic construct
GSDMS

SEQ ID NO: 14 - protein, 16 aa, synthetic construct
YITSGGTTYYPDSVKG

SEQ ID NO: 15 - protein, 12 aa, synthetic construct
SRLTDSHYVEDA

SEQ ID NO: 16 - protein, 5 aa, synthetic construct
TKAMG

SEQ ID NO: 17 - protein, 16 aa, synthetic construct
AISESGSTYYADSVKD

SEQ ID NO: 18 - protein, 15 aa, synthetic construct
KWYSGSFDDTRSYDY

SEQ ID NO: 19 - protein, 5 aa, synthetic construct
FYAMA

SEQ ID NO: 20 - protein, 16 aa, synthetic construct
TITAYGNTNYADSVKG

SEQ ID NO: 21 - protein, 9 aa, synthetic construct
NLPSASHNY

SEQ ID NO: 22 - protein, 120 aa, synthetic construct
QVQLQESGGGMVHPGGSLRLSCAASGFTFSGSDMSWVRQAPGKGPEWVSYITSGGTTYYPDSVKGRFTISRDNAKNTLYLQMNSLTPDDTALYRCAKARLTDSHYVEDAWGQGTQVTVSS

SEQ ID NO: 23 - protein, 121 aa, synthetic construct
QVQLQESGGGMVQVGGSLRLSCAASGFPFSSADMSWVRQAPGKGPEWVSYINADGSKTYYPDSVKGRFTISRDNAKNTLYLLMNNLKPEDTALYRCARAKLTDTHYVEDYWGQGTQVTVSS

SEQ ID NO: 24 - protein, 121 aa, synthetic construct
QVQLQESGGGMVQPGGSLRLSCAASGFTFSNYDMSWVRQAPGKGPEWVSYINAGGSNTDYPDSVKGRFTISRDNAKNMLYLLMNNLKPEDTALYRCARAKLTDTHYVEDYWGQGTQVTVSS

SEQ ID NO: 25 - protein, 120 aa, synthetic construct
QVQLQESGGGLVQPGRSLRLSCAASGFTFSGSDMSWVRQAPGKGPEWVSYITGSGTTYYPDSVKGRFTTSRDNAKNMLYLQMNSLKPDDTALYRCAKARLTDSHYVEDAWGQGTQVTVSS

SEQ ID NO: 26 - protein, 120 aa, synthetic construct
QVQLQESGGGLVEAGGSLRLSCAASGFTFSGSDMSWVRQAPGKGPEWVSYITSGGTTYYPDSVKGRFTISRDNAKNTLYLQMNSLKPDDTALYRCAKSRLTDSHYVEDAWGQGTQVTVSS

SEQ ID NO: 27 - protein, 123 aa, synthetic construct
QVQLQESGGGLVQAGNSLRLSCVASVRTFSTKAMGWFRQAPGKEREFTAAISESGSTYYADSVKDRFTISRDNVKNTVYLQMNSLKPEDTAVYYCGVKWYSGSFDDTRSYDYWGQGTQVTVSS

SEQ ID NO: 28 - protein, 117 aa, synthetic construct
QVQLQESGGGLVQPGGSLRLSCAASGFAFSFYAMAWYRQAPGKQRDMVATITAYGNTNYADSVKGRFTTSRDNAKNTVYLQMNSLKPEDTAVYVCNANLPSASHNYWGQGTQVTVSS

SEQ ID NO: 29 - protein, 352 aa, synthetic construct
QVQLQESGGGMVHPGGSLRLSCAASGFTFSGSDMSWVRQAPGKGPEWVSYITSGGTTYYPDSVKGRFTISRDNAKNTLYLQMNSLTPDDTALYRCAKARLTDSHYVEDAWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 353 aa, synthetic construct
QVQLQESGGGMVQVGGSLRLSCAASGFPFSSADMSWVRQAPGKGPEWVSYINADGSKTYYPDSVKGRFTISRDNAKNTLYLLMNNLKPEDTALYRCARAKLTDTHYVEDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 353 aa, synthetic construct
QVQLQESGGGMVQPGGSLRLSCAASGFTFSNYDMSWVRQAPGKGPEWVSYINAGGSNTDYPDSVKGRFTISRDNAKNMLYLLMNNLKPEDTALYRCARAKLTDTHYVEDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 352 aa, synthetic construct
QVQLQESGGGLVQPGRSLRLSCAASGFTFSGSDMSWVRQAPGKGPEWVSYITGSGTTYYPDSVKGRFTTSRDNAKNMLYLQMNSLKPDDTALYRCAKARLTDSHYVEDAWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 352 aa, synthetic construct
QVQLQESGGGLVEAGGSLRLSCAASGFTFSGSDMSWVRQAPGKGPEWVSYITSGGTTYYPDSVKGRFTISRDNAKNTLYLQMNSLKPDDTALYRCAKSRLTDSHYVEDAWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 355 aa, synthetic construct
QVQLQESGGGLVQAGNSLRLSCVASVRTFSTKAMGWFRQAPGKEREFTAAISESGSTYYADSVKDRFTISRDNVKNTVYLQMNSLKPEDTAVYYCGVKWYSGSFDDTRSYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 349 aa, synthetic construct
QVQLQESGGGLVQPGGSLRLSCAASGFAFSFYAMAWYRQAPGKQRDMVATITAYGNTNYADSVKGRFTTSRDNAKNTVYLQMNSLKPEDTAVYVCNANLPSASHNYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 15 aa, synthetic construct
EPKSCDKTHTCPPCP

SEQ ID NO: 37 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 38 - protein, 106 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 323 aa, synthetic construct
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 40 - protein, 323 aa, synthetic construct
MARGPGLAPPPLRLPLLLLLLAAVTGHTAAQDNCTCPTNKMTVCSPDGPGGRCQCRALGSGVAVDCSTLTSKCLLLKARMSAPKNARTLVRPNEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDVKGESLFQGRGGLDLRVRGEPLQVERTLIYYLDEIPPKFSMKRLTAGLIAVIVVVVVALVAGVAVLVISNRRKSGKYKKVEIKELGELRKEPSL

SEQ ID NO: 41 - protein, 317 aa, synthetic construct
MARGLDLAPLLLLLLAMATRFCTAQSNCTCPTNKMTVCDTNGPGGVCQCRAMGSQVLVDCSTLTSKCLLLKARMSARKSGRSLVMPSEHAILDNDGLYDPECDDKGRFKARQCNQTSVCWCVNSVGVRRTDKGDQSLRCDEVVRTHHILIELRHRPTDRAFNHSDLDSELRRLFQERYKLHPSFLSAVHYEEPTIQIELRQNASQKGLRDVDIADAAYYFERDIKGESLFMGRRGLDVQVRGEPLHVERTLIYYLDEKPPQFSMKRLTAGVIAVIAVVSVAVVAGVVVLVVTKRRKSGKYKKVELKELGEMRSEPSL

SEQ ID NO: 42 - protein, 12 aa, synthetic construct
ESKYGPPCPPCP

SEQ ID NO: 43 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 350 aa, synthetic construct
QVQLQESGGGMVQVGGSLRLSCAASGFPFSSADMSWVRQAPGKGPEWVSYINADGSKTYYPDSVKGRFTISRDNAKNTLYLLMNNLKPEDTALYRCARAKLTDTHYVEDYWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 349 aa, synthetic construct
QVQLQESGGGLVQPGRSLRLSCAASGFTFSGSDMSWVRQAPGKGPEWVSYITGSGTTYYPDSVKGRFTTSRDNAKNMLYLQMNSLKPDDTALYRCAKARLTDSHYVEDAWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 10 aa, synthetic construct
GFPFSSADMS

SEQ ID NO: 47 - protein, 8 aa, synthetic construct
INADGSKT

SEQ ID NO: 48 - protein, 14 aa, synthetic construct
ARAKLTDTHYVEDY

SEQ ID NO: 49 - protein, 10 aa, synthetic construct
GFTFSGSDMS

SEQ ID NO: 50 - protein, 7 aa, synthetic construct
ITSGGTT

SEQ ID NO: 51 - protein, 14 aa, synthetic construct
AKSRLTDSHYVEDA

SEQ ID NO: 52 - protein, 8 aa, synthetic construct
GFTFSGSD

SEQ ID NO: 53 - protein, 7 aa, synthetic construct
ITSGGTT

SEQ ID NO: 54 - protein, 14 aa, synthetic construct
AKARLTDSHYVEDA

SEQ ID NO: 55 - protein, 8 aa, synthetic construct
GFPFSSAD

SEQ ID NO: 56 - protein, 8 aa, synthetic construct
GFTFSNYD

SEQ ID NO: 57 - protein, 8 aa, synthetic construct
INAGGSNT

SEQ ID NO: 58 - protein, 14 aa, synthetic construct
ARAKLTDTHYVEDY

SEQ ID NO: 59 - protein, 8 aa, synthetic construct
GFTFSGSD

SEQ ID NO: 60 - protein, 7 aa, synthetic construct
ITGSGTT

SEQ ID NO: 61 - protein, 14 aa, synthetic construct
AKARLTDSHYVEDA

SEQ ID NO: 62 - protein, 8 aa, synthetic construct
GFTFSGSD

SEQ ID NO: 63 - protein, 8 aa, synthetic construct
VRTFSTKA

SEQ ID NO: 64 - protein, 7 aa, synthetic construct
ISESGST

SEQ ID NO: 65 - protein, 17 aa, synthetic construct
GVKWYSGSFDDTRSYDY

SEQ ID NO: 66 - protein, 8 aa, synthetic construct
GFAFSFYA

SEQ ID NO: 67 - protein, 7 aa, synthetic construct
ITAYGNT

SEQ ID NO: 68 - protein, 11 aa, synthetic construct
NANLPSASHNY

SEQ ID NO: 69-70 - skipped (2 entries)

SEQ ID NO: 71 - protein, 121 aa, synthetic construct
EVQLVESGGGLVQPGGSLRLSCAASGFPFSSADMHWVRQAPGKGLVWVSRINADGSKTSYADTVKGRFTISRDNAKNTLYLQMNSLRAEDTAVYYCARAKLTDTHYVEDYWGQGTLVTVSS

SEQ ID NO: 72 - protein, 121 aa, synthetic construct
EVQLVESGGGLVQPGGSLRLSCAASGFPFSSADMSWVRQAPGKGLEWVSYINADGSKTSYADTVKGRFTISRDNAKNTLYLQMNSLRAEDTAVYYCARAKLTDTHYVEDYWGQGTLVTVSS

SEQ ID NO: 73 - protein, 121 aa, synthetic construct
EVQLVESGGGLVQPGGSLRLSCAASGFPFSSADMSWVRQAPGKGPEWVSYINADGSKTYYADTVKGRFTISRDNAKNTLYLQMNSLRAEDTAVYYCARAKLTDTHYVEDYWGQGTQVTVSS

SEQ ID NO: 74 - protein, 121 aa, synthetic construct
QVQLVESGGGLVQPGGSLRLSCAASGFPFSSADMSWVRQAPGKGPEWVSYINADGSKTYYPDTVKGRFTISRDNAKNTLYLQMNSLRAEDTAVYYCARAKLTDTHYVEDYWGQGTQVTVSS

SEQ ID NO: 75 - protein, 121 aa, synthetic construct
QVQLVESGGGLVQPGGSLRLSCAASGFPFSSADMSWVRQAPGKGPEWVSYINADGSKTYYPDTVKGRFTISRDNAKNTLYLQMNSLRAEDTAVYRCARAKLTDTHYVEDYWGQGTQVTVSS

SEQ ID NO: 76 - protein, 121 aa, synthetic construct
QVQLVESGGGLVQVGGSLRLSCAASGFPFSSADMSWVRQAPGKGPEWVSYINADGSKTYYPDTVKGRFTISRDNAKNTLYLQMNSLKPEDTAVYRCARAKLTDTHYVEDYWGQGTQVTVSS

SEQ ID NO: 77 - protein, 121 aa, synthetic construct
QVQLQESGGGMVQVGGSLRLSCAASGFPFSSADMSWVRQAPGKGPEWVSYINADGSKTYYPDTVKGRFTISRDNAKNTLYLQMNSLKPEDTAVYRCARAKLTDTHYVEDYWGQGTQVTVSS

SEQ ID NO: 78 - protein, 121 aa, synthetic construct
QVQLQESGGGMVQVGGSLRLSCAASGFPFSSADMSWVRQAPGKGPEWVSYINADGSKTYYPDSVKGRFTISRDNAKNTLYLQMNSLKPEDTAVYRCARAKLTDTHYVEDYWGQGTQVTVSS

SEQ ID NO: 79 - protein, 121 aa, synthetic construct
EVQLLESGGGLVQPGGSLRLSCAASGFPFSSADMSWVRQAPGKGLEWVSVINADGSKTYYADTVKGRFTISRDNSKNTLYLQMNSLRAEDTAVYYCARAKLTDTHYVEDYWGQGTMVTVSS

SEQ ID NO: 80 - protein, 121 aa, synthetic construct
QVQLQESGGGLVQPGGSLRLSCAASGFPFSSADMSWVRQAPGKGPEWVSYINADGSKTYYPDTVKGRFTISRDNSKNTLYLQMNNLRAEDTALYRCARAKLTDTHYVEDYWGQGTMVTVSS

SEQ ID NO: 81 - protein, 121 aa, synthetic construct
QVQLQESGGGLVQPGGSLRLSCAASGFPFSSADMSWVRQAPGKGLEWVSYINADGSKTYYADTVKGRFTISRDNSKNTLYLQMNSLRAEDTAVYRCARAKLTDTHYVEDYWGQGTMVTVSS

SEQ ID NO: 82 - protein, 121 aa, synthetic construct
EVQLQESGGGLVQPGGSLRLSCAASGFPFSSADMSWVRQAPGKGLEWVSYINADGSKTYYADTVKGRFTISRDNSKNTLYLQMNSLRAEDTAVYRCARAKLTDTHYVEDYWGQGTMVTVSS

SEQ ID NO: 83 - protein, 121 aa, synthetic construct
QVQLLESGGGLVQPGGSLRLSCAASGFPFSSADMSWVRQAPGKGLEWVSYINADGSKTYYADTVKGRFTISRDNSKNTLYLQMNSLRAEDTAVYRCARAKLTDTHYVEDYWGQGTMVTVSS

SEQ ID NO: 84 - protein, 121 aa, synthetic construct
QVQLQESGGGLVQPGGSLRLSCAASGFPFSSADMSWVRQAPGKGLEWVSVINADGSKTYYADTVKGRFTISRDNSKNTLYLQMNSLRAEDTAVYRCARAKLTDTHYVEDYWGQGTMVTVSS

SEQ ID NO: 85 - protein, 121 aa, synthetic construct
QVQLQESGGGLVQPGGSLRLSCAASGFPFSSADMSWVRQAPGKGLEWVSYINADGSKTYYADTVKGRFTISRDNSKNTLYLQMNSLRAEDTAVYYCARAKLTDTHYVEDYWGQGTMVTVSS

SEQ ID NO: 86 - protein, 121 aa, synthetic construct
QVQLQESGGGLVQPGGSLRLSCAASGFPFSSADMSWVRQAPGKGPEWVSYINADGSKTYYADTVKGRFTISRDNSKNTLYLQMNSLRAEDTAVYRCARAKLTDTHYVEDYWGQGTMVTVSS

SEQ ID NO: 87 - protein, 121 aa, synthetic construct
QVQLQESGGGLVQPGGSLRLSCAASGFPFSSADMSWVRQAPGKGLEWVSYINADGSKTYYPDTVKGRFTISRDNSKNTLYLQMNSLRAEDTAVYRCARAKLTDTHYVEDYWGQGTMVTVSS

SEQ ID NO: 88 - protein, 121 aa, synthetic construct
QVQLQESGGGLVQPGGSLRLSCAASGFPFSSADMSWVRQAPGKGLEWVSYINADGSKTYYADTVKGRFTISRDNSKNTLYLQMNNLRAEDTAVYRCARAKLTDTHYVEDYWGQGTMVTVSS

SEQ ID NO: 89 - protein, 121 aa, synthetic construct
QVQLQESGGGLVQPGGSLRLSCAASGFPFSSADMSWVRQAPGKGLEWVSYINADGSKTYYADTVKGRFTISRDNSKNTLYLQMNSLRAEDTALYRCARAKLTDTHYVEDYWGQGTMVTVSS

SEQ ID NO: 90 - protein, 121 aa, synthetic construct
QVQLQESGGGMVQPGGSLRLSCAASGFPFSSADMSWVRQAPGKGLEWVSYINADGSKTYYADTVKGRFTISRDNSKNTLYLQMNSLRAEDTAVYRCARAKLTDTHYVEDYWGQGTQVTVSS

SEQ ID NO: 91 - protein, 121 aa, synthetic construct
QVQLQESGGGLVQPGGSLRLSCAASGFPFSSADMSWVRQAPGKQREWVSYINADGSKTYYADTVKGRFTISRDNSKNTLYLQMNSLRAEDTAVYRCARAKLTDTHYVEDYWGQGTMVTVSS

SEQ ID NO: 92 - protein, 121 aa, synthetic construct
QVQLQESGGGLVQPGGSLRLSCAASGFPFSSADMSWVRQAPGKGLEWVSYINAEGSKTYYADTVKGRFTISRDNSKNTLYLQMNSLRAEDTAVYRCARAKLTDTHYVEDYWGQGTMVTVSS

SEQ ID NO: 93 - protein, 121 aa, synthetic construct
QVQLQESGGGLVQPGGSLRLSCAASGFPFSSADMSWVRQAPGKGLEWVSYINANGSKTYYADTVKGRFTISRDNSKNTLYLQMNSLRAEDTAVYRCARAKLTDTHYVEDYWGQGTMVTVSS

SEQ ID NO: 94 - protein, 120 aa, synthetic construct
EVQLLESGGGLVQPGGSLRLSCAASGFTFSGSDMSWVRQAPGKGLEWVSAITSGGTTYYADTVKGRFTISRDNSKNTLYLQMNSLRAEDTAVYYCAKSRLTDSHYVEDAWGQGTLVTVSS

SEQ ID NO: 95 - protein, 120 aa, synthetic construct
EVQLLESGGGLVQPGGSLRLSCAASGFTFSGSDMSWVRQAPGKGLEWVSYITSGGTTYYADTVKGRFTISRDNSKNTLYLQMNSLRAEDTAVYYCAKSRLTDSHYVEDAWGQGTLVTVSS

SEQ ID NO: 96 - protein, 120 aa, synthetic construct
EVQLLESGGGLVQPGGSLRLSCAASGFTFSGSDMSWVRQAPGKGLEWVSYITSGGTTYYADTVKGRFTISRDNSKNTLYLQMNSLRAEDTAVYRCAKSRLTDSHYVEDAWGQGTLVTVSS

SEQ ID NO: 97 - protein, 120 aa, synthetic construct
QVQLLESGGGLVQPGGSLRLSCAASGFTFSGSDMSWVRQAPGKGPEWVSYITSGGTTYYADTVKGRFTISRDNSKNTLYLQMNSLRAEDTAVYYCAKSRLTDSHYVEDAWGQGTLVTVSS

SEQ ID NO: 98 - protein, 120 aa, synthetic construct
QVQLLESGGGLVQPGGSLRLSCAASGFTFSGSDMSWVRQAPGKGPEWVSYITSGGTTYYADTVKGRFTISRDNSKNTLYLQMNSLRAEDTAVYRCAKSRLTDSHYVEDAWGQGTLVTVSS

SEQ ID NO: 99 - protein, 120 aa, synthetic construct
QVQLQESGGGLVQPGGSLRLSCAASGFTFSGSDMSWVRQAPGKGPEWVSYITSGGTTYYPDTVKGRFTISRDNSKNTLYLQMNSLRAEDTAVYYCAKSRLTDSHYVEDAWGQGTQVTVSS

SEQ ID NO: 100 - protein, 120 aa, synthetic construct
QVQLQESGGGLVQPGGSLRLSCAASGFTFSGSDMSWVRQAPGKGPEWVSYITSGGTTYYPDTVKGRFTISRDNSKNTLYLQMNSLRAEDTAVYRCAKSRLTDSHYVEDAWGQGTQVTVSS

SEQ ID NO: 101 - protein, 120 aa, synthetic construct
QVQLQESGGGLVQPGGSLRLSCAASGFTFSGSDMSWVRQAPGKGPEWVSYITSGGTTYYPDTVKGRFTISRDNSKNTLYLQMNSLKPDDTAVYRCAKSRLTDSHYVEDAWGQGTQVTVSS

SEQ ID NO: 102 - protein, 120 aa, synthetic construct
QVQLQESGGGLVQPGGSLRLSCAASGFTFSGSDMSWVRQAPGKGPEWVSYITSGGTTYYPDTVKGRFTISRDNAKNTLYLQMNSLKPDDTALYRCAKSRLTDSHYVEDAWGQGTQVTVSS

SEQ ID NO: 103 - protein, 120 aa, synthetic construct
EVQLVESGGGLIQPGGSLRLSCAASGFTFSGSDMSWVRQAPGKGLEWVSVITSGGTTYYADTVKGRFTISRDNSKNTLYLQMNSLRAEDTAVYYCAKSRLTDSHYVEDAWGQGTLVTVSS

SEQ ID NO: 104 - protein, 120 aa, synthetic construct
QVQLQESGGGLIQPGGSLRLSCAASGFTFSGSDMSWVRQAPGKGPEWVSYITSGGTTYYPDTVKGRFTISRDNSKNTLYLQMNSLRADDTALYRCAKSRLTDSHYVEDAWGQGTLVTVSS

SEQ ID NO: 105 - protein, 120 aa, synthetic construct
QVQLQESGGGLIQPGGSLRLSCAASGFTFSGSDMSWVRQAPGKGLEWVSYITSGGTTYYADTVKGRFTISRDNSKNTLYLQMNSLRAEDTAVYRCAKSRLTDSHYVEDAWGQGTLVTVSS

SEQ ID NO: 106 - protein, 120 aa, synthetic construct
EVQLQESGGGLIQPGGSLRLSCAASGFTFSGSDMSWVRQAPGKGLEWVSYITSGGTTYYADTVKGRFTISRDNSKNTLYLQMNSLRAEDTAVYRCAKSRLTDSHYVEDAWGQGTLVTVSS

SEQ ID NO: 107 - protein, 120 aa, synthetic construct
QVQLVESGGGLIQPGGSLRLSCAASGFTFSGSDMSWVRQAPGKGLEWVSYITSGGTTYYADTVKGRFTISRDNSKNTLYLQMNSLRAEDTAVYRCAKSRLTDSHYVEDAWGQGTLVTVSS

SEQ ID NO: 108 - protein, 120 aa, synthetic construct
QVQLQESGGGLIQPGGSLRLSCAASGFTFSGSDMSWVRQAPGKGLEWVSVITSGGTTYYADTVKGRFTISRDNSKNTLYLQMNSLRAEDTAVYRCAKSRLTDSHYVEDAWGQGTLVTVSS

SEQ ID NO: 109 - protein, 120 aa, synthetic construct
QVQLQESGGGLIQPGGSLRLSCAASGFTFSGSDMSWVRQAPGKGLEWVSYITSGGTTYYADTVKGRFTISRDNSKNTLYLQMNSLRAEDTAVYYCAKSRLTDSHYVEDAWGQGTLVTVSS

SEQ ID NO: 110 - protein, 120 aa, synthetic construct
QVQLQESGGGLIQPGGSLRLSCAASGFTFSGSDMSWVRQAPGKGPEWVSYITSGGTTYYADTVKGRFTISRDNSKNTLYLQMNSLRAEDTAVYRCAKSRLTDSHYVEDAWGQGTLVTVSS

SEQ ID NO: 111 - protein, 120 aa, synthetic construct
QVQLQESGGGLIQPGGSLRLSCAASGFTFSGSDMSWVRQAPGKGLEWVSYITSGGTTYYPDTVKGRFTISRDNSKNTLYLQMNSLRAEDTAVYRCAKSRLTDSHYVEDAWGQGTLVTVSS

SEQ ID NO: 112 - protein, 120 aa, synthetic construct
QVQLQESGGGLIQPGGSLRLSCAASGFTFSGSDMSWVRQAPGKGLEWVSYITSGGTTYYADTVKGRFTISRDNSKNTLYLQMNSLRADDTAVYRCAKSRLTDSHYVEDAWGQGTLVTVSS

SEQ ID NO: 113 - protein, 120 aa, synthetic construct
QVQLQESGGGLIQPGGSLRLSCAASGFTFSGSDMSWVRQAPGKGLEWVSYITSGGTTYYADTVKGRFTISRDNSKNTLYLQMNSLRAEDTALYRCAKSRLTDSHYVEDAWGQGTLVTVSS

SEQ ID NO: 114 - protein, 120 aa, synthetic construct
QVQLQESGGGLIEPGGSLRLSCAASGFTFSGSDMSWVRQAPGKGLEWVSYITSGGTTYYADTVKGRFTISRDNSKNTLYLQMNSLRAEDTAVYRCAKSRLTDSHYVEDAWGQGTQVTVSS

SEQ ID NO: 115 - protein, 120 aa, synthetic construct
QVQLQESGGGLIQPGGSLRLSCAASGFTFSGSDMSWYRQAPGKGREWVSYITSGGTTYYADTVKGRFTISRDNSKNTLYLQMNSLRAEDTAVYRCAKSRLTDSHYVEDAWGQGTLVTVSS

SEQ ID NO: 116 - protein, 17 aa, synthetic construct
MSWVRQAPGKGPEWVSY

SEQ ID NO: 117 - protein, 17 aa, synthetic construct
MSWVRQAPGKGLEWVSY

SEQ ID NO: 118 - protein, 17 aa, synthetic construct
MSWVRQAPGKGLEWVSV

SEQ ID NO: 119 - protein, 17 aa, synthetic construct
MSWVRQAPGKQREWVSY

SEQ ID NO: 120 - protein, 17 aa, synthetic construct
MSWVRQAPGKGLEWVSA

SEQ ID NO: 121 - protein, 17 aa, synthetic construct
  VARIANT 11..17: note = Xaa is any amino acid
MSWVRQAPGKXXEWVSX

SEQ ID NO: 122 - protein, 8 aa, synthetic construct
  VARIANT 3..7: note = Xaa is any amino acid
GFXXSXXD

SEQ ID NO: 123 - protein, 8 aa, synthetic construct
INANGSKT

SEQ ID NO: 124 - protein, 8 aa, synthetic construct
INAEGSKT

SEQ ID NO: 125 - skipped

SEQ ID NO: 126 - protein, 14 aa, synthetic construct
  VARIANT 2..14: note = Xaa is any amino acid
AXXXLTDXHYVEDX

SEQ ID NO: 127 - protein, 5 aa, synthetic construct
SADMH

SEQ ID NO: 128 - skipped

SEQ ID NO: 129 - protein, 17 aa, synthetic construct
RINADGSKTSYADTVKG

SEQ ID NO: 130 - protein, 17 aa, synthetic construct
YINADGSKTSYADTVKG

SEQ ID NO: 131 - protein, 17 aa, synthetic construct
YINADGSKTYYPDTVKG

SEQ ID NO: 132 - protein, 17 aa, synthetic construct
VINADGSKTYYADTVKG

SEQ ID NO: 133 - protein, 17 aa, synthetic construct
YINADGSKTYYADTVKG

SEQ ID NO: 134 - protein, 17 aa, synthetic construct
YINAEGSKTYYADTVKG

SEQ ID NO: 135 - protein, 17 aa, synthetic construct
YINANGSKTYYADTVKG

SEQ ID NO: 136 - protein, 16 aa, synthetic construct
AITSGGTTYYADTVKG

SEQ ID NO: 137 - protein, 16 aa, synthetic construct
YITSGGTTYYADTVKG

SEQ ID NO: 138 - protein, 16 aa, synthetic construct
YITSGGTTYYPDTVKG

SEQ ID NO: 139 - protein, 16 aa, synthetic construct
VITSGGTTYYADTVKG

SEQ ID NO: 140 - protein, 17 aa, synthetic construct
  VARIANT 1..14: note = Xaa is any amino acid
XIXXXXXXTXYXDXVKG

SEQ ID NO: 141 - protein, 12 aa, synthetic construct
SRLTDSHYVEDA

SEQ ID NO: 142 - protein, 12 aa, synthetic construct
  VARIANT 1..12: note = Xaa is any amino acid
XXLTDXHYVEDX

SEQ ID NO: 143 - protein, 121 aa, synthetic construct
  VARIANT 1..116: note = Xaa is any amino acid
XVQLXESGGGXVQXGGSLRLSCAASGFPFSSADMSWVRQAPGKXXEWVSXINAXGSKTYYXDXVKGRFTISRDNXKNTLYLXMNXLXXEDTAXYXCARAKLTDTHYVEDYWGQGTXVTVSS

SEQ ID NO: 144 - protein, 120 aa, synthetic construct
  VARIANT 1..115: note = Xaa is any amino acid
XVQLXESGGGLXXXGGSLRLSCAASGFTFSGSDMSWVRQAPGKGXEWVSXITSGGTTYYXDXVKGRFTISRDNXKNTLYLQMNSLXXXDTAXYXCAKSRLTDSHYVEDAWGQGTXVTVSS

SEQ ID NO: 145 - protein, 121 aa, synthetic construct
  VARIANT 11..110: note = Xaa is any amino acid
QVQLQESGGGXVXXGXSLRLSCAASGFXFSXXDMSWVRQAPGKGPEWVSYIXXXXXXTXYPDSVKGRFTXSRDNAKNXLYLXMNXLXPXDTALYRCAXXXLTDXHYVEDXWGQGTQVTVSS

SEQ ID NO: 146 - protein, 121 aa, synthetic construct
  VARIANT 1..116: note = Xaa is any amino acid
XVQLXESGGGXXXXGXSLRLSCAASGFXFSXXDMXWXRQAPGKXXXWVSXIXXXGXXTXYXDXVKGRFTXSRDNXKNXLYLXMNXLXXXDTAXYXCAXXXLTDXHYVEDXWGQGTXVTVSS

SEQ ID NO: 147 - protein, 313 aa, synthetic construct
MARGPGLAPPPLRLPLLLLVLAAVTGPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDDLVRTHHILIDLRHRPTAGAFNHSDLDAELRRLFRERYRLHPKFVAAVHYEQPTIQIELRQNTSQKAAGEVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 148 - protein, 304 aa, synthetic construct
MARGPGLAPPPLRLPLLLLVLAAVTGSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDDLVRTHHILIDLRHRPTAGAFNHSDLDAELRRLFRERYRLHPKFVAAVHYEQPTIQIELRQNTSQKAAGEVDIGDAAYYFERDIKGESLFQGRGGLDLRVRGEPLQVERTLIYYLDEIPPKFSMKRLTAGLIAVIVVVVVALVAGMAVLVITNRKSGKYKKVEIKELGELRKEPSL

SEQ ID NO: 149 - protein, 293 aa, synthetic construct
MARGPGLAPPPLRLPLLLLVLAAVTGALGSGMAVDCSTLTSKCLLLKARMSAPKNARTLVRPSEHALVDNDGLYDPDCDPEGRFKARQCNQTSVCWCVNSVGVRRTDKGDLSLRCDDLVRTHHILIDLRHRPTAGAFNHSDLDAELRRLFRERYRLHPKFVAAVHYEQPTIQIELRQNTSQKAAGEVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 150 - protein, 485 aa, synthetic construct
QVQLQESGGGLVQPGRSLRLSCAASGFTFSGSDMSWVRQAPGKGPEWVSYITGSGTTYYPDSVKGRFTTSRDNAKNMLYLQMNSLKPDDTALYRCAKARLTDSHYVEDAWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPAPAPAPAPAPKAQVQLQESGGGLVQPGGSLRLSCAASRFDFSVNDIRWYRQAPGNERELVARITGGGRTDYADSVKGRFTISRDNAKNTVYLQMNNLKPEDTAVYYCWGRGYWGQGTQVTVSS

SEQ ID NO: 151 - protein, 485 aa, synthetic construct
QVQLQESGGGLVQPGGSLRLSCAASRFDFSVNDIRWYRQAPGNERELVARITGGGRTDYADSVKGRFTISRDNAKNTVYLQMNNLKPEDTAVYYCWGRGYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPAPAPAPAPAPKAQVQLQESGGGLVQPGRSLRLSCAASGFTFSGSDMSWVRQAPGKGPEWVSYITGSGTTYYPDSVKGRFTTSRDNAKNMLYLQMNSLKPDDTALYRCAKARLTDSHYVEDAWGQGTQVTVSS

SEQ ID NO: 152 - protein, 348 aa, synthetic construct
QVQLQESGGGMVQVGGSLRLSCAASGFPFSSADMSWVRQAPGKGPEWVSYINADGSKTYYPDSVKGRFTISRDNAKNTLYLLMNNLKPEDTALYRCARAKLTDTHYVEDYWGQGTQVTVSSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 153 - protein, 347 aa, synthetic construct
QVQLQESGGGLVEAGGSLRLSCAASGFTFSGSDMSWVRQAPGKGPEWVSYITSGGTTYYPDSVKGRFTISRDNAKNTLYLQMNSLKPDDTALYRCAKSRLTDSHYVEDAWGQGTQVTVSSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 154 - protein, 354 aa, synthetic construct
DVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 155 - protein, 111 aa, synthetic construct
QVQLQESGGGLVQPGGSLRLSCAASRFDFSVNDIRWYRQAPGNERELVARITGGGRTDYADSVKGRFTISRDNAKNTVYLQMNNLKPEDTAVYYCWGRGYWGQGTQVTVSS

SEQ ID NO: 156 - protein, 5 aa, synthetic construct
VNDIR

SEQ ID NO: 157 - protein, 16 aa, synthetic construct
RITGGGRTDYADSVKG

SEQ ID NO: 158 - protein, 5 aa, synthetic construct
WGRGY

SEQ ID NO: 159 - protein, 8 aa, synthetic construct
RFDFSVND

SEQ ID NO: 160 - protein, 7 aa, synthetic construct
ITGGGRT

SEQ ID NO: 161 - protein, 5 aa, synthetic construct
WGRGY

SEQ ID NO: 162 - protein, 349 aa, synthetic construct
QVQLQESGGGLVEAGGSLRLSCAASGFTFSGSDMSWVRQAPGKGPEWVSYITSGGTTYYPDSVKGRFTISRDNAKNTLYLQMNSLKPDDTALYRCAKSRLTDSHYVEDAWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - protein, 22 aa, synthetic construct
APAPAPAPAPAPAPAPAPAPKA

SEQ ID NO: 164 - protein, 274 aa, synthetic construct
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLT

SEQ ID NO: 165 - DNA, 1056 bp, synthetic construct
  source 1..1056: mol_type = other DNA
caggtgcaattgcaagagtctggcggcggaatggtgcaccctggtggatctctgagactgtcttgtgccgcctccggcttcaccttctctggctctgatatgtcctgggtccgacaggctcctggcaagggacctgaatgggtgtcctacatcacctccggcggcaccacctactatcccgactctgtgaagggcagattcaccatctctcgggacaacgccaagaacaccctgtacctgcagatgaactctctgacccctgacgacacagccctgtacagatgtgccaaggccagactgaccgacagccactacgtggaagatgcttggggccagggaacacaggtcaccgtgtctagtgaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaa

SEQ ID NO: 166 - DNA, 1059 bp, synthetic construct
  source 1..1059: mol_type = other DNA
caggtgcaattgcaagagtctggcggcggaatggtgcaagtcggcggatctctgagactgtcttgtgccgcctctggcttccctttctcctccgccgatatgtcctgggtccgacaggctcctggaaagggacctgagtgggtgtcctacatcaacgccgacggctccaagacctactatcccgactctgtgaagggcagattcaccatctctcgggacaacgccaagaacaccctgtacctgctgatgaacaacctgaagcctgaggacacagccctgtacagatgcgccagagccaagctgaccgacacacactacgtggaagattactggggccagggcacacaggtcaccgtgtctagtgaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaa

SEQ ID NO: 167 - DNA, 1059 bp, synthetic construct
  source 1..1059: mol_type = other DNA
caggtgcaattgcaagagtctggcggcggaatggtgcagcctggtggatctctgagactgtcttgtgccgcctccggcttcaccttctccaactacgacatgtcctgggtccgacaggctcctggaaagggacctgagtgggtgtcctacatcaacgctggcggctccaacaccgactatcccgactctgtgaagggcagattcaccatctctcgggacaacgccaagaacatgctgtacctgctgatgaacaacctgaagcctgaggacacagccctgtacagatgcgccagagccaagctgaccgacacacactacgtggaagattactggggccagggcacacaggtcaccgtgtctagtgaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaa

SEQ ID NO: 168 - DNA, 1056 bp, synthetic construct
  source 1..1056: mol_type = other DNA
caggtgcaattgcaagagtctggcggcggactggtgcagcctggaagatctctgagactgtcttgtgccgcctccggcttcaccttctctggctctgatatgtcctgggtccgacaggctcctggcaagggacctgaatgggtgtcctacatcaccggctccggcaccacctactatcccgactctgtgaagggcagattcaccacctccagagacaacgccaagaacatgctgtacctgcagatgaactccctgaagcctgacgacacagccctgtacagatgcgccaaggccagactgaccgacagccactacgtggaagatgcttggggccagggaacacaggtcaccgtgtctagtgaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaa

SEQ ID NO: 169 - DNA, 1056 bp, synthetic construct
  source 1..1056: mol_type = other DNA
caggtgcaattgcaagagtctggcggcggactggtggaagctggtggatctctgagactgtcttgtgccgcctccggcttcaccttctctggctctgatatgtcctgggtccgacaggctcctggcaagggacctgaatgggtgtcctacatcacctccggcggcaccacctactatcccgactctgtgaagggcagattcaccatctctcgggacaacgccaagaacaccctgtacctgcagatgaactccctgaagcctgacgacacagccctgtacagatgcgccaagtctcggctgaccgacagccactacgtggaagatgcttggggccagggaacacaggtcaccgtgtctagtgaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaa

SEQ ID NO: 170 - protein, 14 aa, synthetic construct
WVRQAPGKGPEWVS

SEQ ID NO: 171 - protein, 14 aa, synthetic construct
WVRQAPGKGLEWVS

SEQ ID NO: 172 - protein, 14 aa, synthetic construct
WVRQAPGKQREWVS

SEQ ID NO: 173 - protein, 14 aa, synthetic construct
WYRQAPGKGREWVS

SEQ ID NO: 174 - protein, 14 aa, synthetic construct
WVRQAPGKGLVWVS

SEQ ID NO: 175 - protein, 14 aa, synthetic construct
  VARIANT 2..11: note = Xaa is any amino acid
WXRQAPGKXXXWVS

SEQ ID NO: 176 - protein, 139 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSP

SEQ ID NO: 177 - protein, 350 aa, synthetic construct; identical to SEQ ID NO: 44

SEQ ID NO: 178 - protein, 349 aa, synthetic construct; identical to SEQ ID NO: 45

SEQ ID NO: 179 - protein, 110 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 180 - protein, 17 aa, synthetic construct
  VARIANT 11: note = Q, G or W
  VARIANT 12: note = R, L or P
  VARIANT 17: note = Y, A or V
MSWVRQAPGKXXEWVSX

SEQ ID NO: 181 - protein, 8 aa, synthetic construct
  VARIANT 6: note = G, N, S or D
  VARIANT 3: note = Xaa is any amino acid
  VARIANT 7: note = Xaa is any amino acid
  VARIANT 4: note = Xaa is F, V, L, I or M
GFXXSXXD

SEQ ID NO: 182 - protein, 8 aa, synthetic construct
  VARIANT 3: note = T or P
  VARIANT 6: note = G, N or S
  VARIANT 7: note = S, Y or A
  VARIANT 4: note = Xaa is F or L
GFXXSXXD

SEQ ID NO: 183-184 - skipped (2 entries)

SEQ ID NO: 185 - protein, 14 aa, synthetic construct
  VARIANT 2: note = Q, H, R or K
  VARIANT 3: note = S, T or A
  VARIANT 4: note = Q, H, R or K
  VARIANT 8: note = any amino acid or S, T or A
  VARIANT 14: note = any amino acid or absent
AXXXLTDXHYVEDX

SEQ ID NO: 186 - protein, 14 aa, synthetic construct
  VARIANT 2: note = R or K
  VARIANT 3: note = A or S
  VARIANT 4: note = K or R
  VARIANT 8: note = T, S or G
  VARIANT 14: note = Y, A or absent
AXXXLTDXHYVEDX

SEQ ID NO: 187-188 - skipped (2 entries)

SEQ ID NO: 189 - protein, 17 aa, synthetic construct
  VARIANT 3: note = any amino acid or absent
  VARIANT 4: note = S, T or A
  VARIANT 6: note = S, A or G
  VARIANT 7: note = S, A or G
  VARIANT 8: note = S, T, N or K
  VARIANT 14: note = S, T or A
  VARIANT 1: note = Xaa is any amino acid
  VARIANT 5: note = Xaa is any amino acid
  VARIANT 10: note = Xaa is any amino acid
  VARIANT 12: note = Xaa is any amino acid
XIXXXXXXTXYXDXVKG

SEQ ID NO: 190 - protein, 17 aa, synthetic construct
  VARIANT 1: note = Y, A, V or R
  VARIANT 3: note = absent or N
  VARIANT 4: note = T or A
  VARIANT 5: note = G, S, D, N or E
  VARIANT 6: note = S or G
  VARIANT 7: note = G or S
  VARIANT 8: note = T, N or K
  VARIANT 10: note = Y, D or S
  VARIANT 12: note = P or A
  VARIANT 14: note = S or T
XIXXXXXXTXYXDXVKG

SEQ ID NO: 191 - protein, 12 aa, synthetic construct
  VARIANT 1: note = S, T or A
  VARIANT 2: note = Q, H, R or K
  VARIANT 6: note = S, T or A
  VARIANT 12: note = any amino acid or absent
XXLTDXHYVEDX

SEQ ID NO: 192 - protein, 12 aa, synthetic construct
  VARIANT 1: note = A or S
  VARIANT 2: note = K or R
  VARIANT 6: note = T or S
  VARIANT 12: note = Y, A or absent
XXLTDXHYVEDX

SEQ ID NO: 193 - protein, 14 aa, synthetic construct
  VARIANT 2: note = V or Y
  VARIANT 9: note = G or Q
  VARIANT 10: note = P, L or R
  VARIANT 11: note = E or V
WXRQAPGKXXXWVS

SEQ ID NO: 194 - protein, 14 aa, synthetic construct
  VARIANT 9: note = G or Q
  VARIANT 10: note = P, L or R
WVRQAPGKXXEWVS

SEQ ID NO: 195 - protein, 121 aa, synthetic construct
  VARIANT 11: note = M or L
  VARIANT 14: note = V, P or A
  VARIANT 54: note = D, G or S
  VARIANT 82: note = L or Q
  VARIANT 85: note = N or S
  VARIANT 87: note = K or T
QVQLQESGGGXVQXGGSLRLSCAASGFPFSSADMSWVRQAPGKGPEWVSYINAXGSKTYYPDSVKGRFTISRDNAKNTLYLXMNXLXPEDTALYRCARAKLTDTHYVEDYWGQGTQVTVSS

SEQ ID NO: 196 - protein, 121 aa, synthetic construct; identical to SEQ ID NO: 143
  VARIANT 1: note = Q or E
  VARIANT 5: note = Q, V or L
  VARIANT 11: note = M or L
  VARIANT 14: note = V or P
  VARIANT 44: note = G or Q
  VARIANT 45: note = P, L or R
  VARIANT 50: note = Y or V
  VARIANT 54: note = D, N or E
  VARIANT 61: note = P or A
  VARIANT 63: note = S or T
  VARIANT 75: note = A or S
  VARIANT 82: note = L or Q
  VARIANT 85: note = N or S
  VARIANT 87: note = K or R
  VARIANT 88: note = P or A
  VARIANT 93: note = L or V
  VARIANT 95: note = R or Y
  VARIANT 116: note = Q or M

SEQ ID NO: 197 - protein, 121 aa, synthetic construct
  VARIANT 1: note = Q or E
  VARIANT 5: note = Q, V or L
  VARIANT 11: note = M or L
  VARIANT 14: note = V or P
  VARIANT 44: note = G or Q
  VARIANT 45: note = P, L or R
  VARIANT 61: note = P or A
  VARIANT 63: note = S or T
  VARIANT 75: note = A or S
  VARIANT 82: note = L or Q
  VARIANT 85: note = N or S
  VARIANT 87: note = K or R
  VARIANT 88: note = P or A
  VARIANT 93: note = L or V
  VARIANT 95: note = R or Y
  VARIANT 116: note = Q or M
XVQLXESGGGXVQXGGSLRLSCAASGFPFSSADMSWVRQAPGKXXEWVSYINADGSKTYYXDXVKGRFTISRDNXKNTLYLXMNXLXXEDTAXYXCARAKLTDTHYVEDYWGQGTXVTVSS

SEQ ID NO: 198 - protein, 120 aa, synthetic construct
  VARIANT 13: note = Q, H or E
  VARIANT 14: note = V, P or A
  VARIANT 86: note = K or T
  VARIANT 88: note = E or D
QVQLQESGGGLVXXGGSLRLSCAASGFTFSGSDMSWVRQAPGKGPEWVSYITSGGTTYYPDSVKGRFTISRDNAKNTLYLQMNSLXPXDTALYRCAKSRLTDSHYVEDAWGQGTQVTVSS

SEQ ID NO: 199 - protein, 120 aa, synthetic construct; identical to SEQ ID NO: 144
  VARIANT 1: note = Q or E
  VARIANT 5: note = Q, L or V
  VARIANT 12: note = V or I
  VARIANT 13: note = E or Q
  VARIANT 14: note = A or P
  VARIANT 45: note = P or L
  VARIANT 50: note = Y, A or V
  VARIANT 60: note = P or A
  VARIANT 62: note = S or T
  VARIANT 74: note = A or S
  VARIANT 86: note = K or R
  VARIANT 87: note = P or A
  VARIANT 115: note = Q or L
  VARIANT 92: note = L or V
  VARIANT 94: note = R or Y
  VARIANT 88: note = D or E

SEQ ID NO: 200 - protein, 120 aa, synthetic construct; identical to SEQ ID NO: 144
  VARIANT 1: note = Q or E
  VARIANT 5: note = Q or L
  VARIANT 12: note = V or I
  VARIANT 13: note = E or Q
  VARIANT 14: note = A or P
  VARIANT 45: note = P or L
  VARIANT 50: note = Y or A
  VARIANT 60: note = P or A
  VARIANT 62: note = S or T
  VARIANT 74: note = A or S
  VARIANT 86: note = K or R
  VARIANT 87: note = P or A
  VARIANT 88: note = D or E
  VARIANT 92: note = L or V
  VARIANT 94: note = R or Y
  VARIANT 115: note = Q or L

SEQ ID NO: 201 - protein, 121 aa, synthetic construct; identical to SEQ ID NO: 145
  VARIANT 11: note = M or L
  VARIANT 13: note = Q, H or E
  VARIANT 14: note = V, P or A
  VARIANT 16: note = G or R
  VARIANT 28: note = P or T
  VARIANT 31: note = S, N or G
  VARIANT 32: note = A, Y or S
  VARIANT 52: note = N or T
  VARIANT 53: note = A, S or G
  VARIANT 54: note = D, G or S
  VARIANT 55: note = G or absent
  VARIANT 56: note = S or G
  VARIANT 57: note = K, N or T
  VARIANT 59: note = Y or D
  VARIANT 70: note = I or T
  VARIANT 78: note = T or M
  VARIANT 82: note = L or Q
  VARIANT 85: note = N or S
  VARIANT 87: note = K or T
  VARIANT 89: note = E or D
  VARIANT 98: note = R or K
  VARIANT 99: note = A or S
  VARIANT 100: note = K or R
  VARIANT 104: note = T or S
  VARIANT 110: note = Y or A

SEQ ID NO: 202 - protein, 121 aa, synthetic construct; identical to SEQ ID NO: 145
  VARIANT 11: note = M or L
  VARIANT 13: note = Q, H or E
  VARIANT 14: note = V, P or A
  VARIANT 16: note = G or R
  VARIANT 28: note = P or T
  VARIANT 31: note = S, N or G
  VARIANT 32: note = A, Y or S
  VARIANT 52: note = N or T
  VARIANT 53: note = A, S or G
  VARIANT 54: note = D, G or S
  VARIANT 55: note = G or absent
  VARIANT 56: note = S or G
  VARIANT 57: note = K, N or T
  VARIANT 59: note = Y or D
  VARIANT 70: note = I or T
  VARIANT 78: note = T or M
  VARIANT 82: note = L or Q
  VARIANT 85: note = N or S
  VARIANT 87: note = K or T
  VARIANT 89: note = E or D
  VARIANT 98: note = R or K
  VARIANT 99: note = A or S
  VARIANT 100: note = K or R
  VARIANT 104: note = T or S
  VARIANT 110: note = Y or A

SEQ ID NO: 203 - protein, 121 aa, synthetic construct
  VARIANT 11: note = M or L
  VARIANT 13: note = Q, H or E
  VARIANT 14: note = V, P or A
  VARIANT 16: note = G or R
  VARIANT 28: note = P or T
  VARIANT 31: note = S, N or G
  VARIANT 32: note = A, Y or S
  VARIANT 52: note = N or T
  VARIANT 53: note = A, S or G
  VARIANT 54: note = D, G or S
  VARIANT 55: note = G or absent
  VARIANT 56: note = S or G
  VARIANT 57: note = K, N or T
  VARIANT 70: note = I or T
  VARIANT 78: note = T or M
  VARIANT 82: note = L or Q
  VARIANT 85: note = N or S
  VARIANT 87: note = K or T
  VARIANT 89: note = E or D
  VARIANT 98: note = R or K
  VARIANT 99: note = A or S
QVQLQESGGGXVXXGXSLRLSCAASGFXFSXXDMSWVRQAPGKGPEWVSYIXXXXXXTYYPDSVKGRFTXSRDNAKNXLYLXMNXLXPXDTALYRCAXXRLTDSHYVEDAWGQGTQVTVSS

SEQ ID NO: 204 - protein, 121 aa, synthetic construct
  VARIANT 11: note = M or L
  VARIANT 13: note = Q, H or E
  VARIANT 14: note = V, P or A
  VARIANT 16: note = G or R
  VARIANT 28: note = P or T
  VARIANT 31: note = S, N or G
  VARIANT 32: note = A, Y or S
  VARIANT 52: note = N or T
  VARIANT 53: note = A, S or G
  VARIANT 54: note = D, G or S
  VARIANT 56: note = S or G
  VARIANT 57: note = K, N or T
  VARIANT 59: note = Y or D
  VARIANT 70: note = I or T
  VARIANT 78: note = T or M
  VARIANT 82: note = L or Q
  VARIANT 85: note = N or S
  VARIANT 87: note = K or T
  VARIANT 89: note = E or D
  VARIANT 98: note = R or K
  VARIANT 99: note = A or S
  VARIANT 100: note = K or R
  VARIANT 104: note = T or S
  VARIANT 110: note = Y or A
QVQLQESGGGXVXXGXSLRLSCAASGFXFSXXDMSWVRQAPGKGPEWVSYIXXXGXXTXYPDSVKGRFTXSRDNAKNXLYLXMNXLXPXDTALYRCAXXXLTDXHYVEDXWGQGTQVTVSS

SEQ ID NO: 205 - protein, 121 aa, synthetic construct; identical to SEQ ID NO: 146
  VARIANT 1: note = Q or E
  VARIANT 5: note = Q, L or V
  VARIANT 11: note = M or L
  VARIANT 12: note = V or I
  VARIANT 13: note = Q, H or E
  VARIANT 14: note = P, A or V
  VARIANT 16: note = G or R
  VARIANT 28: note = P or T
  VARIANT 31: note = G, S or N
  VARIANT 32: note = A or S
  VARIANT 35: note = S or H
  VARIANT 37: note = V or Y
  VARIANT 44: note = G or Q
  VARIANT 45: note = P, L or R
  VARIANT 46: note = E or V
  VARIANT 50: note = Y, A or R
  VARIANT 52: note = T or N
  VARIANT 53: note = A, S or G
  VARIANT 54: note = G, S, D, N or E
  VARIANT 56: note = absent or S
  VARIANT 57: note = K or T
  VARIANT 59: note = Y, D or S
  VARIANT 61: note = P or A
  VARIANT 63: note = S or T
  VARIANT 70: note = I or T
  VARIANT 75: note = S or A
  VARIANT 78: note = T or M
  VARIANT 82: note = Q or L
  VARIANT 85: note = S or N
  VARIANT 87: note = K, R or T
  VARIANT 88: note = P or A
  VARIANT 89: note = E or D
  VARIANT 93: note = V or L
  VARIANT 95: note = Y or R
  VARIANT 99: note = A or S
  VARIANT 100: note = K or R
  VARIANT 104: note = S or T
  VARIANT 110: note = Y or A
  VARIANT 116: note = Q, L or M
  VARIANT 98: note = R or K

SEQ ID NO: 206 - protein, 5 aa, synthetic construct
GGGGS

SEQ ID NO: 207 - protein, 8 aa, synthetic construct
  REPEAT 2..6: note = amino acid residues 2-6 can be repeated from one to ten times
  VARIANT 1: note = Xaa is absent or A
XPAPAPKA

SEQ ID NO: 208 - protein, 121 aa, synthetic construct
  VARIANT 1: note = Q or E
  VARIANT 5: note = Q, V or L
  VARIANT 11: note = M or L
  VARIANT 14: note = V or P
  VARIANT 44: note = G or Q
  VARIANT 45: note = P, L or R
  VARIANT 61: note = P or A
  VARIANT 63: note = S or T
  VARIANT 75: note = A or S
  VARIANT 87: note = K or R
  VARIANT 88: note = P or A
  VARIANT 93: note = L or V
  VARIANT 95: note = R or Y
  VARIANT 116: note = Q or M
XVQLXESGGGXVQXGGSLRLSCAASGFPFSSADMSWVRQAPGKXXEWVSYINADGSKTYYXDXVKGRFTISRDNXKNTLYLQMNSLXXEDTAXYXCARAKLTDTHYVEDYWGQGTXVTVSS

SEQ ID NO: 209 - protein, 120 aa, synthetic construct
  VARIANT 1: note = Q or E
  VARIANT 5: note = Q, L or V
  VARIANT 12: note = V or I
  VARIANT 45: note = P or L
  VARIANT 50: note = Y, A or V
  VARIANT 60: note = P or A
  VARIANT 74: note = A or S
  VARIANT 86: note = K or R
  VARIANT 87: note = P or A
  VARIANT 88: note = D or E
  VARIANT 92: note = L or V
  VARIANT 94: note = R or Y
  VARIANT 115: note = Q or L
XVQLXESGGGLXQPGGSLRLSCAASGFTFSGSDMSWVRQAPGKGXEWVSXITSGGTTYYXDTVKGRFTISRDNXKNTLYLQMNSLXXXDTAXYXCAKSRLTDSHYVEDAWGQGTXVTVSS

SEQ ID NO: 210 - protein, 120 aa, synthetic construct
  VARIANT 1: note = Q or E
  VARIANT 5: note = Q, L or V
  VARIANT 12: note = V or I
  VARIANT 13: note = E or Q
  VARIANT 45: note = P or L
  VARIANT 50: note = Y, A or V
  VARIANT 60: note = P or A
  VARIANT 74: note = A or S
  VARIANT 86: note = K or R
  VARIANT 87: note = P or A
  VARIANT 88: note = D or E
  VARIANT 92: note = L or V
  VARIANT 94: note = R or Y
  VARIANT 115: note = Q or L
XVQLXESGGGLXXPGGSLRLSCAASGFTFSGSDMSWVRQAPGKGXEWVSXITSGGTTYYXDTVKGRFTISRDNXKNTLYLQMNSLXXXDTAXYXCAKSRLTDSHYVEDAWGQGTXVTVSS

SEQ ID NO: 211 - protein, 94 aa, synthetic construct
  VARIANT 1..89: note = Xaa is any amino acid
XVQLXESGGGXVQXGGSLRLSCAASXMSWVRQAPGKXXEWVSXXYYXDXVKGRFTISRDNXKNTLYLXMNXLXXEDTAXYXCXWGQGTXVTVSS

SEQ ID NO: 212 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 94 aa, synthetic construct
  VARIANT 1..89: note = Xaa is any amino acid
XVQLXESGGGXXXXGGSLRLSCXASXMSWVRQAPGKGXEWVSXXYYXDXVKGRFTISRDNXKNTLYLQMNSLXXXDTAXYXCXXGQGTXVTVSS

SEQ ID NO: 214 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 215 - protein, 5 aa, synthetic construct
GSDMS

SEQ ID NO: 216 - protein, 16 aa, synthetic construct
YITSGGTTYYPDSVKG

SEQ ID NO: 217 - protein, 12 aa, synthetic construct
ARLTDSHYVEDA

SEQ ID NO: 218 - protein, 8 aa, synthetic construct
GFTFSGSD

SEQ ID NO: 219 - protein, 7 aa, synthetic construct
ITSGGTT

SEQ ID NO: 220 - protein, 14 aa, synthetic construct
AKARLTDSHYVEDA

SEQ ID NO: 221 - protein, 120 aa, synthetic construct
QVQLQESGGGLVQAGGSLRLSCAASGFTFSGSDMSWVRQAPGKGPEWVSYITSGGTTYYPDSVKGRFTISRDNAKNTLYLQMNSLKPDDTALYRCAKARLTDSHYVEDAWGQGTQVTVSS

SEQ ID NO: 222 - protein, 120 aa, synthetic construct
QVQLQESGGGLVQPGGSLRLSCAASGFTFSGSDMSWVRQAPGKGPEWVSYITSGGTTYYPDSVKGRFTISRDNAKNTLYLQMNSLKPDDTALYRCAKARLTDSHYVEDAWGQGTQVTVSS

SEQ ID NO: 223 - protein, 120 aa, synthetic construct
QVQLQESGGGLVQPGGSLRLSCAASGFTFSGSDMSWVRQAPGKGPEWVSYITSGGTTYYPDSVKGRFTISRDNAKNTLYLQMNSLTPDDTALYRCAKARLTDSHYVEDAWGQGTQVTVSS

SEQ ID NO: 224 - protein, 120 aa, synthetic construct
QVQLQESGGGLVHPGGSLRLSCAASGFTFSGSDMSWVRQAPGKGPEWVSYITSGGTTYYPDSVKGRFTISRDNAKNTLYLQMNSLKPDDTALYRCAKARLTDSHYVEDAWGQGTQVTVSS

SEQ ID NO: 225 - protein, 5 aa, synthetic construct
GSDMS

SEQ ID NO: 226 - protein, 16 aa, synthetic construct
YITGGGTTYYPDSVKG

SEQ ID NO: 227 - protein, 12 aa, synthetic construct
ARLTDSHYVEDA

SEQ ID NO: 228 - protein, 8 aa, synthetic construct
GFTFSGSD

SEQ ID NO: 229 - protein, 7 aa, synthetic construct
ITGGGTT

SEQ ID NO: 230 - protein, 14 aa, synthetic construct
AKARLTDSHYVEDA

SEQ ID NO: 231 - protein, 120 aa, synthetic construct
QVQLQESGGGLVQAGGSLRLSCAASGFTFSGSDMSWVRQAPGKGPEWVSYITGGGTTYYPDSVKGRFTISRDNAKNTLYLQMNSLKPDDTALYRCAKARLTDSHYVEDAWGQGTQVTVSS

SEQ ID NO: 232 - protein, 120 aa, synthetic construct
QVQLQESGGGLVQPGGSLRLSCAASGFTFSGSDMSWVRQAPGKGPEWVSYITGGGTTYYPDSVKGRFTISRDNAKNTLYLQMNSLKPDDTALYRCAKARLTDSHYVEDAWGQGTQVTVSS

SEQ ID NO: 233 - protein, 5 aa, synthetic construct
GSDMS

SEQ ID NO: 234 - protein, 16 aa, synthetic construct
YITAGGTTYYPDSVKG

SEQ ID NO: 235 - protein, 12 aa, synthetic construct
ARLTDSHYVEDA

SEQ ID NO: 236 - protein, 8 aa, synthetic construct
GFTFSGSD

SEQ ID NO: 237 - protein, 7 aa, synthetic construct
ITAGGTT

SEQ ID NO: 238 - protein, 14 aa, synthetic construct
AKARLTDSHYVEDA

SEQ ID NO: 239 - protein, 120 aa, synthetic construct
QVQLQESGGGLVQAGGSLRLSCAASGFTFSGSDMSWVRQAPGKGPEWVSYITAGGTTYYPDSVKGRFTISRDNAKNTLYLQMNSLKPDDTALYRCAKARLTDSHYVEDAWGQGTQVTVSS

SEQ ID NO: 240 - protein, 120 aa, synthetic construct
QVQLQESGGGLVQPGGSLRLSCAASGFTFSGSDMSWVRQAPGKGPEWVSYITAGGTTYYPDSVKGRFTISRDNAKNTLYLQMNSLKPDDTALYRCAKARLTDSHYVEDAWGQGTQVTVSS

SEQ ID NO: 241 - protein, 5 aa, synthetic construct
GSDMS

SEQ ID NO: 242 - protein, 16 aa, synthetic construct
YITSGGGTYYPDSVKG

SEQ ID NO: 243 - protein, 12 aa, synthetic construct
ARLTDSHYVEDA

SEQ ID NO: 244 - protein, 8 aa, synthetic construct
GFTFSGSD

SEQ ID NO: 245 - protein, 7 aa, synthetic construct
ITSGGGT

SEQ ID NO: 246 - protein, 14 aa, synthetic construct
AKARLTDSHYVEDA

SEQ ID NO: 247 - protein, 120 aa, synthetic construct
QVQLQESGGGLVQPGGSLRLSCAASGFTFSGSDMSWVRQAPGKGPEWVSYITSGGGTYYPDSVKGRFTISRDNAKNTLYLQMNSLKPEDTALYRCAKARLTDSHYVEDAWGQGTQVTVSS

SEQ ID NO: 248 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGMVQVGGSLRLSCAASXMSWVRQAPGKGPEWVSYXYYPDSVKGRFTISRDNAKNTLYLLMNNLKPEDTALYRCXWGQGTQVTVSS

SEQ ID NO: 249 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
EVQLVESGGGLVQPGGSLRLSCAASXMHWVRQAPGKGLVWVSRXSYADTVKGRFTISRDNAKNTLYLQMNSLRAEDTAVYYCXWGQGTLVTVSS

SEQ ID NO: 250 - protein, 94 aa, synthetic construct
  MOTIF 26: note = X is an amino acid sequence of IMGT CDR1
  MOTIF 44: note = X is an amino acid sequence of IMGT CDR2
  MOTIF 86: note = X is an amino acid sequence of IMGT CDR3
EVQLVESGGGLVQPGGSLRLSCAASXMSWVRQAPGKGLEWVSYXSYADTVKGRFTISRDNAKNTLYLQMNSLRAEDTAVYYCXWGQGTLVTVSS

SEQ ID NO: 251 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
EVQLVESGGGLVQPGGSLRLSCAASXMSWVRQAPGKGPEWVSYXYYADTVKGRFTISRDNAKNTLYLQMNSLRAEDTAVYYCXWGQGTQVTVSS

SEQ ID NO: 252 - protein, 94 aa, synthetic construct
  MOTIF 26: note = X is an amino acid sequence of IMGT CDR1
  MOTIF 44
  MOTIF 84: note = X is an amino acid sequence of IMGT CDR3
QVQLVESGGGLVQPGGSLRLSCAASXMSWVRQAPGKGPEWVSYXYYPDTVKGRFTISRDNAKNTLYLQMNSLRAEDTAVYYCXWGQGTQVTVSS

SEQ ID NO: 253 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLVESGGGLVQPGGSLRLSCAASXMSWVRQAPGKGPEWVSYXYYPDTVKGRFTISRDNAKNTLYLQMNSLRAEDTAVYRCXWGQGTQVTVSS

SEQ ID NO: 254 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLVESGGGLVQVGGSLRLSCAASXMSWVRQAPGKGPEWVSYXYYPDTVKGRFTISRDNAKNTLYLQMNSLKPEDTAVYRCXWGQGTQVTVSS

SEQ ID NO: 255 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGMVQVGGSLRLSCAASXMSWVRQAPGKGPEWVSYXYYPDTVKGRFTISRDNAKNTLYLQMNSLKPEDTAVYRCXWGQGTQVTVSS

SEQ ID NO: 256 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGMVQVGGSLRLSCAASXMSWVRQAPGKGPEWVSYXYYPDSVKGRFTISRDNAKNTLYLQMNSLKPEDTAVYRCXWGQGTQVTVSS

SEQ ID NO: 257 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
EVQLLESGGGLVQPGGSLRLSCAASXMSWVRQAPGKGLEWVSVXYYADTVKGRFTISRDNSKNTLYLQMNSLRAEDTAVYYCXWGQGTMVTVSS

SEQ ID NO: 258 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGPEWVSYXYYPDTVKGRFTISRDNSKNTLYLQMNNLRAEDTALYRCXWGQGTMVTVSS

SEQ ID NO: 259 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGLEWVSYXYYADTVKGRFTISRDNSKNTLYLQMNSLRAEDTAVYRCXWGQGTMVTVSS

SEQ ID NO: 260 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
EVQLQESGGGLVQPGGSLRLSCAASXMSWVRQAPGKGLEWVSYXYYADTVKGRFTISRDNSKNTLYLQMNSLRAEDTAVYRCXWGQGTMVTVSS

SEQ ID NO: 261 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLLESGGGLVQPGGSLRLSCAASXMSWVRQAPGKGLEWVSYXYYADTVKGRFTISRDNSKNTLYLQMNSLRAEDTAVYRCXWGQGTMVTVSS

SEQ ID NO: 262 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGLEWVSVXYYADTVKGRFTISRDNSKNTLYLQMNSLRAEDTAVYRCXWGQGTMVTVSS

SEQ ID NO: 263 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGLEWVSYXYYADTVKGRFTISRDNSKNTLYLQMNSLRAEDTAVYYCXWGQGTMVTVSS

SEQ ID NO: 264 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGPEWVSYXYYADTVKGRFTISRDNSKNTLYLQMNSLRAEDTAVYRCXWGQGTMVTVSS

SEQ ID NO: 265 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGLEWVSYXYYPDTVKGRFTISRDNSKNTLYLQMNSLRAEDTAVYRCXWGQGTMVTVSS

SEQ ID NO: 266 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGLEWVSYXYYADTVKGRFTISRDNSKNTLYLQMNNLRAEDTAVYRCXWGQGTMVTVSS

SEQ ID NO: 267 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGLEWVSYXYYADTVKGRFTISRDNSKNTLYLQMNSLRAEDTALYRCXWGQGTMVTVSS

SEQ ID NO: 268 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGMVQPGGSLRLSCAASXMSWVRQAPGKGLEWVSYXYYADTVKGRFTISRDNSKNTLYLQMNSLRAEDTAVYRCXWGQGTQVTVSS

SEQ ID NO: 269 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKQREWVSYXYYADTVKGRFTISRDNSKNTLYLQMNSLRAEDTAVYRCXWGQGTMVTVSS

SEQ ID NO: 270 - protein, 94 aa, synthetic construct; identical to SEQ ID NO: 259
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3

SEQ ID NO: 271 - protein, 94 aa, synthetic construct; identical to SEQ ID NO: 259
  MOTIF 26: note = X is an amino acid sequnce of IMGT CDR1
  MOTIF 44: note = X is an amino acid sequence of IMGT CDR2
  MOTIF 83: note = X is an amino acid sequence of IMGT CDR3

SEQ ID NO: 272 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVEAGGSLRLSCAASXMSWVRQAPGKGPEWVSYXYYPDSVKGRFTISRDNAKNTLYLQMNSLKPDDTALYRCXWGQGTQVTVSS

SEQ ID NO: 273 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
EVQLLESGGGLVQPGGSLRLSCAASXMSWVRQAPGKGLEWVSAXYYADTVKGRFTISRDNSKNTLYLQMNSLRAEDTAVYYCXWGQGTLVTVSS

SEQ ID NO: 274 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
EVQLLESGGGLVQPGGSLRLSCAASXMSWVRQAPGKGLEWVSYXYYADTVKGRFTISRDNSKNTLYLQMNSLRAEDTAVYYCXWGQGTLVTVSS

SEQ ID NO: 275 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
EVQLLESGGGLVQPGGSLRLSCAASXMSWVRQAPGKGLEWVSYXYYADTVKGRFTISRDNSKNTLYLQMNSLRAEDTAVYRCXWGQGTLVTVSS

SEQ ID NO: 276 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLLESGGGLVQPGGSLRLSCAASXMSWVRQAPGKGPEWVSYXYYADTVKGRFTISRDNSKNTLYLQMNSLRAEDTAVYYCXWGQGTLVTVSS

SEQ ID NO: 277 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLLESGGGLVQPGGSLRLSCAASXMSWVRQAPGKGPEWVSYXYYADTVKGRFTISRDNSKNTLYLQMNSLRAEDTAVYRCXWGQGTLVTVSS

SEQ ID NO: 278 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGPEWVSYXYYPDTVKGRFTISRDNSKNTLYLQMNSLRAEDTAVYYCXWGQGTQVTVSS

SEQ ID NO: 279 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGPEWVSYXYYPDTVKGRFTISRDNSKNTLYLQMNSLRAEDTAVYRCXWGQGTQVTVSS

SEQ ID NO: 280 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGPEWVSYXYYPDTVKGRFTISRDNSKNTLYLQMNSLKPDDTAVYRCXWGQGTQVTVSS

SEQ ID NO: 281 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVQPGGSLRLSCAASXMSWVRQAPGKGPEWVSYXYYPDTVKGRFTISRDNAKNTLYLQMNSLKPDDTALYRCXWGQGTQVTVSS

SEQ ID NO: 282 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
EVQLVESGGGLIQPGGSLRLSCAASXMSWVRQAPGKGLEWVSVXYYADTVKGRFTISRDNSKNTLYLQMNSLRAEDTAVYYCXWGQGTLVTVSS

SEQ ID NO: 283 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIQPGGSLRLSCAASXMSWVRQAPGKGPEWVSYXYYPDTVKGRFTISRDNSKNTLYLQMNSLRADDTALYRCXWGQGTLVTVSS

SEQ ID NO: 284 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIQPGGSLRLSCAASXMSWVRQAPGKGLEWVSYXYYADTVKGRFTISRDNSKNTLYLQMNSLRAEDTAVYRCXWGQGTLVTVSS

SEQ ID NO: 285 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
EVQLQESGGGLIQPGGSLRLSCAASXMSWVRQAPGKGLEWVSYXYYADTVKGRFTISRDNSKNTLYLQMNSLRAEDTAVYRCXWGQGTLVTVSS

SEQ ID NO: 286 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLVESGGGLIQPGGSLRLSCAASXMSWVRQAPGKGLEWVSYXYYADTVKGRFTISRDNSKNTLYLQMNSLRAEDTAVYRCXWGQGTLVTVSS

SEQ ID NO: 287 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIQPGGSLRLSCAASXMSWVRQAPGKGLEWVSVXYYADTVKGRFTISRDNSKNTLYLQMNSLRAEDTAVYRCXWGQGTLVTVSS

SEQ ID NO: 288 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIQPGGSLRLSCAASXMSWVRQAPGKGLEWVSYXYYADTVKGRFTISRDNSKNTLYLQMNSLRAEDTAVYYCXWGQGTLVTVSS

SEQ ID NO: 289 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIQPGGSLRLSCAASXMSWVRQAPGKGPEWVSYXYYADTVKGRFTISRDNSKNTLYLQMNSLRAEDTAVYRCXWGQGTLVTVSS

SEQ ID NO: 290 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIQPGGSLRLSCAASXMSWVRQAPGKGLEWVSYXYYPDTVKGRFTISRDNSKNTLYLQMNSLRAEDTAVYRCXWGQGTLVTVSS

SEQ ID NO: 291 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIQPGGSLRLSCAASXMSWVRQAPGKGLEWVSYXYYADTVKGRFTISRDNSKNTLYLQMNSLRADDTAVYRCXWGQGTLVTVSS

SEQ ID NO: 292 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIQPGGSLRLSCAASXMSWVRQAPGKGLEWVSYXYYADTVKGRFTISRDNSKNTLYLQMNSLRAEDTALYRCXWGQGTLVTVSS

SEQ ID NO: 293 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIEPGGSLRLSCAASXMSWVRQAPGKGLEWVSYXYYADTVKGRFTISRDNSKNTLYLQMNSLRAEDTAVYRCXWGQGTQVTVSS

SEQ ID NO: 294 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLIQPGGSLRLSCAASXMSWYRQAPGKGREWVSYXYYADTVKGRFTISRDNSKNTLYLQMNSLRAEDTAVYRCXWGQGTLVTVSS

SEQ ID NO: 295 - protein, 120 aa, synthetic construct
QVQLQESGGGLVQPGGSLRLSCAASGFTFSGSDMSWVRQAPGKGPEWVSYITGGGTTYYPDSVKGRFTISRDNANNMLYLQMNSLKPEDTALYRCAKARLTDSHYVEDAWGQGTQVTVSS

SEQ ID NO: 296 - protein, 120 aa, synthetic construct
QVQLQESGGGMVQPGGSLRLSCAASGFTFSGSDMSWVRQAPGKGPEWVSYITGGGTTYYPDSVKGRFTISRDNANNMLYLQMNSLKPEDTALYRCAKARLTDSHYVEDAWGQGTQVTVSS

SEQ ID NO: 297 - protein, 120 aa, synthetic construct
QVQLQESGGGMVHPGGSLRLSCAASGFTFSGSDMSWVRQAPGKGPEWVSYITGGGTTYYPDSVKGRFTISRDNANNMLYLQMNSRKPEDTAVYRCAKARLTDSHYVEDAWGQGTQVTVSS

SEQ ID NO: 298 - protein, 5 aa, synthetic construct
GSDMS

SEQ ID NO: 299 - protein, 16 aa, synthetic construct
YITGSGTTYYPDSVKG

SEQ ID NO: 300 - protein, 12 aa, synthetic construct
ARLTDSHYVEDA

SEQ ID NO: 301 - protein, 8 aa, synthetic construct
GFTFSGSD

SEQ ID NO: 302 - protein, 7 aa, synthetic construct
ITGSGTT

SEQ ID NO: 303 - protein, 14 aa, synthetic construct
AKARLTDSHYVEDA

SEQ ID NO: 304 - protein, 120 aa, synthetic construct
QVQLQESGGGLVQPGGSLRLSCAASGFTFSGSDMSWVRQAPGKGPEWVSYITGSGTTYYPDSVKGRFTISRDNAKNTLYLQMNSLKPDDTALYRCAKARLTDSHYVEDAWGQGTQVTVSS

SEQ ID NO: 305 - protein, 5 aa, synthetic construct
SYDMS

SEQ ID NO: 306 - protein, 17 aa, synthetic construct
TINTGGGSTYYADSVKG

SEQ ID NO: 307 - protein, 12 aa, synthetic construct
ARLTDSHYVEDA

SEQ ID NO: 308 - protein, 8 aa, synthetic construct
GFTFSSYD

SEQ ID NO: 309 - protein, 8 aa, synthetic construct
INTGGGST

SEQ ID NO: 310 - protein, 14 aa, synthetic construct
AKARLTDSHYVEDA

SEQ ID NO: 311 - protein, 121 aa, synthetic construct
QVQLQESGGGMVQPGGSLRLSCAASGFTFSSYDMSWVRQAPGKGPEWVSTINTGGGSTYYADSVKGRFTISRDNANNMLYLQMNSLKPEDTALYRCAKARLTDSHYVEDAWGQGTQVTVSS

SEQ ID NO: 312 - protein, 5 aa, synthetic construct
SYDMS

SEQ ID NO: 313 - protein, 17 aa, synthetic construct
TINTDGGSTYYADSVKD

SEQ ID NO: 314 - protein, 12 aa, synthetic construct
ARLTDSHYVEDA

SEQ ID NO: 315 - protein, 8 aa, synthetic construct
GFTFSSYD

SEQ ID NO: 316 - protein, 8 aa, synthetic construct
INTDGGST

SEQ ID NO: 317 - protein, 14 aa, synthetic construct
AKARLTDSHYVEDA

SEQ ID NO: 318 - protein, 121 aa, synthetic construct
QVQLQESGGGMVQPGGSLRLSCAASGFTFSSYDMSWVRQAPGKGPEWLSTINTDGGSTYYADSVKDRFTISRDNAKNTLYLQMNSLTPDDTALYRCAKARLTDSHYVEDAWGQGTQVTVSS

SEQ ID NO: 319 - protein, 5 aa, synthetic construct
GSDMS

SEQ ID NO: 320 - protein, 16 aa, synthetic construct
YITGGGGTYYADSVKG

SEQ ID NO: 321 - protein, 12 aa, synthetic construct
ARLTDSHYVEDA

SEQ ID NO: 322 - protein, 8 aa, synthetic construct
GFTFSGSD

SEQ ID NO: 323 - protein, 7 aa, synthetic construct
ITGGGGT

SEQ ID NO: 324 - protein, 14 aa, synthetic construct
AKARLTDSHYVEDA

SEQ ID NO: 325 - protein, 120 aa, synthetic construct
QVQLQESGGGMVHPGGSLRLSCAASGFTFSGSDMSWVRQAPGKGPEWVSYITGGGGTYYADSVKGRFTISRDHAKNMLYLQMNSLKPEDTALYRCAKARLTDSHYVEDAWGQGTQVTVSS

SEQ ID NO: 326 - protein, 5 aa, synthetic construct
DSDMS

SEQ ID NO: 327 - protein, 16 aa, synthetic construct
YITGGGGTYYADSVKG

SEQ ID NO: 328 - protein, 12 aa, synthetic construct
ARLTDSHYVEDA

SEQ ID NO: 329 - protein, 8 aa, synthetic construct
GFTFSDSD

SEQ ID NO: 330 - protein, 7 aa, synthetic construct
ITGGGGT

SEQ ID NO: 331 - protein, 14 aa, synthetic construct
AKARLTDSHYVEDA

SEQ ID NO: 332 - protein, 120 aa, synthetic construct
QVQLQESGGGLVQPGGSLRLSCAASGFTFSDSDMSCVRQAPGTGPEWVSYITGGGGTYYADSVKGRFTISRDNAKNMLYLQMNSLKPEDTALYRCAKARLTDSHYVEDAWGQGTQVTVSS

SEQ ID NO: 333 - protein, 5 aa, synthetic construct
SADMS

SEQ ID NO: 334 - protein, 17 aa, synthetic construct
YINADGSKTYYPDSVKG

SEQ ID NO: 335 - protein, 12 aa, synthetic construct
ARLTDSHYVEDA

SEQ ID NO: 336 - protein, 8 aa, synthetic construct
GFPFSSAD

SEQ ID NO: 337 - protein, 8 aa, synthetic construct
INADGSKT

SEQ ID NO: 338 - protein, 14 aa, synthetic construct
AKARLTDSHYVEDA

SEQ ID NO: 339 - protein, 121 aa, synthetic construct
QVQLQESGGGMVQVGGSLRLSCAASGFPFSSADMSWVRQAPGKGPEWVSYINADGSKTYYPDSVKGRFTISRDNAKNTLYLLMNNLRPEDTALYRCAKARLTDSHYVEDAWGQGTQVTVSS

SEQ ID NO: 340 - protein, 5 aa, synthetic construct
GSDMS

SEQ ID NO: 341 - protein, 16 aa, synthetic construct
SLTSGGTTYYPDSVKG

SEQ ID NO: 342 - protein, 12 aa, synthetic construct
ARLTDSHYVEDA

SEQ ID NO: 343 - protein, 8 aa, synthetic construct
GFTFSGSD

SEQ ID NO: 344 - protein, 7 aa, synthetic construct
LTSGGTT

SEQ ID NO: 345 - protein, 14 aa, synthetic construct
AKARLTDSHYVEDA

SEQ ID NO: 346 - protein, 120 aa, synthetic construct
QVQLQESGGGMVHPGGSLRLSCAASGFTFSGSDMSWVRQVPGQGPEWVSSLTSGGTTYYPDSVKGRFSISRDNAKNTLYLQMNSLKPDDTALYRCAKARLTDSHYVEDAWGQGTHVTVSS

SEQ ID NO: 347 - protein, 5 aa, synthetic construct
NYAMS

SEQ ID NO: 348 - protein, 16 aa, synthetic construct
YITSGGGTYYPDSVKG

SEQ ID NO: 349 - protein, 12 aa, synthetic construct
ARLTDSHYVEDA

SEQ ID NO: 350 - protein, 8 aa, synthetic construct
GFSFNNYA

SEQ ID NO: 351 - protein, 7 aa, synthetic construct
ITSGGGT

SEQ ID NO: 352 - protein, 14 aa, synthetic construct
AKARLTDSHYVEDA

SEQ ID NO: 353 - protein, 120 aa, synthetic construct
QVQLQESGGGLVQPGGSLRLSCAASGFSFNNYAMSWVRQAPGKGPEWVSYITSGGGTYYPDSVKGRFTISRDNAKNTLYLQMNSLKPEDTALYRCAKARLTDSHYVEDAWGQGTQVTVSS

SEQ ID NO: 354 - protein, 17 aa, synthetic construct
MSWVRQAPGKGPEWLST

SEQ ID NO: 355 - protein, 17 aa, synthetic construct
MSCVRQAPGTGPEWVSY

SEQ ID NO: 356 - protein, 17 aa, synthetic construct
MSWVRQVPGQGPEWVSS

SEQ ID NO: 357 - protein, 14 aa, synthetic construct
WVRQAPGKGPEWLS

SEQ ID NO: 358 - protein, 14 aa, synthetic construct
CVRQAPGTGPEWVS

SEQ ID NO: 359 - protein, 14 aa, synthetic construct
WVRQVPGQGPEWVS

SEQ ID NO: 360 - protein, 17 aa, synthetic construct
MSWVRQAPGKGPEWVST

SEQ ID NO: 361 - protein, 17 aa, synthetic construct
MSWVRQAPGKGPEWGSY

SEQ ID NO: 362 - protein, 120 aa, synthetic construct; identical to SEQ ID NO: 22

SEQ ID NO: 363 - protein, 120 aa, synthetic construct
QVQLQESGGGMVHPGGSLRLSCAASGFTFSGSDMSWVRQAPGKGPEWVSYITSGGTTYYPDSVKGRFTISRDNAKNTLYLQMNSLKPDDTALYRCAKARLTDSHYVEDAWGQGTQVTVSS

SEQ ID NO: 364 - protein, 120 aa, synthetic construct
QVQLQESGGGMVDPGGSLRLSCVASGFTFSGSDMSWVRQAPGKGPEWVSYITSGGTTYYPDSVKGRFTISRDNAKNTLYLQMNSLKPDDTALYRCAKARLTDSHYVEDAWGQGTQVTVSS

SEQ ID NO: 365 - protein, 120 aa, synthetic construct
QVQLQESGGGLVQPGGSLRLSCAASGFTFSGSDMSWVRQAPGKGPEWVSYITSGGTTYYPDSVKGRFTISRDNANNMLYLQMNSLKPEDTALYRCAKARLTDSHYVEDAWGQGTQVTVSS

SEQ ID NO: 366 - protein, 120 aa, synthetic construct
QVQLQESGGGLVQPGDSLRLSCAASGFTFSGSDMSWVRQAPGKGPEWVSYITSGGTTYYPDSVKGRFTISRDNAKNTLYLQMNSLKPDDTALYRCAKARLTDSHYVEDAWGQGTQVTVSS

SEQ ID NO: 367 - protein, 120 aa, synthetic construct
QVQLQESGGGMVHPGGSLRLSCAASGFTFSGSDMSWVRQAPGKGPEWVSYITSGGTTYYPDSVKGRFTISRDNAKNTLYLQMNSLKPDDTALDRCAKARLTDSHYVEDAWGQGTQVTVSS

SEQ ID NO: 368 - protein, 120 aa, synthetic construct
QVQLQESGGGMVHPGGSLRLSCAASGFTFSGSDMSWVRQAPGKGPEWVSYITSGGTTYYPDSVKGRFTISRDNANNMLYLQMNSLKPEDTALYRCAKARLTDSHYVEDAWGQGTQVTVSS

SEQ ID NO: 369 - protein, 120 aa, synthetic construct
QVQLQESGGGLVHPGGSLRLSCAASGFTFSGSDMSWVRQAPGKGPEWVSYITSGGTTYYPDSVKGRFTISRDNAKNTLYLQMNSLKPDDTALYRCAKARLTDSHYVEDAQGQGTQVTVSS

SEQ ID NO: 370 - protein, 120 aa, synthetic construct
QVQLQESGGGMVHPGGSLRLSCAASGFTFSGSDMSWVRQAPGKGPEWVSYITSGGTTYYPDSVKGRFTISRDNAKNTLYLQMNSLKPDDTALYRCAKARLTDSHYVEDAQGQGTQVTVSS

SEQ ID NO: 371 - protein, 120 aa, synthetic construct
QVQLQESGGGMVHPGGSLRLSCAASGFTFSGSDMSWVRQAPGKGPEWVSYITSGGTTYYPDSVKGRFTISRDNAKNTLYLQMNSLTPDDTALYRCAKARLTDSHYVEDAQGQGTQVTVSS

SEQ ID NO: 372 - protein, 120 aa, synthetic construct
QVQLQESGGGMVHPGGSLRLSCVASGFTFSGSDMSWVRQAPGKGPEWVSYITGGGTTYYPDSVKGRFTISRDNAKNTLYLQMNSLKPDDTALYRCAKARLTDSHYVEDAWGQGTQVTVSS

SEQ ID NO: 373 - protein, 120 aa, synthetic construct
QVQLQESGGGMVHPGGSLRLSCAASGFTFSGSDMSWVRQAPGKGPEWVSYITGGGTTYYPDSVKGRFTISRDNAKNTLYLQMNSLKPDDTALYRCAKARLTDSHYVEDAWGQGTQVTVSS

SEQ ID NO: 374 - protein, 120 aa, synthetic construct
QVQLQESGGGMVHPGGSLRLSCAASGFTFSGSDMSWVRQAPGKGPEWVSYITGGGTTYYPDSVKGRFTISRDNANNMLYLQMNSLKPEDTALYRCAKARLTDSHYVEDAWGQGTQVTVSS

SEQ ID NO: 375 - protein, 120 aa, synthetic construct
QVQLQESGGGMVHPGGSLRLSCAASGFTFSGSDMSWVRQAPGKGPEWGSYITGGGTTYYPDSVKGRFTISRDNAKNTLYLQMNSLKPDDTALYRCAKARLTDSHYVEDAWGQGTQVTVSS

SEQ ID NO: 376 - protein, 120 aa, synthetic construct
QVQLQESGGGLVHPGGSLRLSCAASGFTFSGSDMSWVRQAPGKGPEWVSYITGGGTTYYPDSVKGRFTISRDNAKNTLYLQMNSLKPDDTALYRCAKARLTDSHYVEDAWGQGTQVTVSS

SEQ ID NO: 377 - protein, 120 aa, synthetic construct
QVQLQESGGGLVQAGGSLRLSCVASGFTFSGSDMSWVRQAPGKGPEWVSYITGGGTTYYPDSVKGRFTISRDNAKNTLYLQMNSLKPDDTALYRCAKARLTDSHYVEDAWGQGTQVTVSS

SEQ ID NO: 378 - protein, 120 aa, synthetic construct
QVQLQESGGGLVQAGGSLRLSCAASGFTFSGSDMSWVRQAPGKGPEWVSYITGGGTTYYPDSVKGRFTISRDNANNMLYLQMNSLKPEDTALYRCAKARLTDSHYVEDAWGQGTQVTVSS

SEQ ID NO: 379 - protein, 120 aa, synthetic construct
QVQLQESGGGLVQPGGSLRLSCVASGFTFSGSDMSWVRQAPGKGPEWVSYITGGGTTYYPDSVKGRFTISRDNAKNTLYLQMNSLKPDDTALYRCAKARLTDSHYVEDAWGQGTQVTVSS

SEQ ID NO: 380 - protein, 120 aa, synthetic construct
QVQLQESGGGMVHPGGSLRLSCAASGFTFSGSDMSWVRQAPGKGPEWVSYITGGGTTYYPDSVKGRFTISRDNAKNTLYLQMNSLKPDDTALYRCAKARLTDSHYVEDAQGQGTQVTVSS

SEQ ID NO: 381 - protein, 120 aa, synthetic construct
QVQLQESGGGMVQPGGSLRLSCAASGFTFSGSDMSWVRQAPGKGPEWVSYITGGGTTYYPDSVKGRFTISRDNAKNTLYLQMNSLKPDDTALYRCAKARLTDSHYVEDAQGQGTQVTVSS

SEQ ID NO: 382 - protein, 120 aa, synthetic construct
QVQLQESGGGMVHPGGSLRLSCVASGFTFSGSDMSWVRQAPGKGPEWVSYITGGGTTYYPDSVKGRFTISRDNAKNTLYLQMNSLKPDDTALYRCAKARLTDSHYVEDAQGQGTQVTVSS

SEQ ID NO: 383 - protein, 120 aa, synthetic construct
QVQLQESGGGMVHPGGSLRLSCAASGFTFSGSDMSWVRQAPGKGPEWVSYITAGGTTYYPDSVKGRFTISRDNAKNTLYLQMNSLKPDDTALYRCAKARLTDSHYVEDAQGQGTQVTVSS

SEQ ID NO: 384 - protein, 120 aa, synthetic construct
QVQLQESGGGLVQPGGSLRLSCAASGFTFSGSDMSWVRQAPGKGPEWVSYITAGGTTYYPDSVKGRFTISRDNAKNTLYLQMNSLKPDDTALYRCAKARLTDSHYVEDAQGQGTQVTVSS

SEQ ID NO: 385 - protein, 120 aa, synthetic construct
QVQLQESGGGLVQPGGSLRLSCVASGFTFSGSDMSWVRQAPGKGPEWVSYITAGGTTYYPDSVKGRFTISRDNAKNTLYLQMNSLKPDDTALYRCAKARLTDSHYVEDAWGQGTQVTVSS

SEQ ID NO: 386 - protein, 120 aa, synthetic construct
QVQLQESGGGLVHPGGSLRLSCAASGFTFSGSDMSWVRQAPGKGPEWVSYITAGGTTYYPDSVKGRFTISRDNAKNTLYLQMNSLKPDDTALYRCAKARLTDSHYVEDAWGQGTQVTVSS

SEQ ID NO: 387 - protein, 120 aa, synthetic construct
QVQLQESGGGMVHPGGSLRLSCAASGFTFSGSDMSWVRQAPGKGPEWVSYITAGGTTYYPDSVKGRFTISRDNAKNTLYLQMNSLKPDDTALYRCAKARLTDSHYVEDAWGQGTQVTVSS

SEQ ID NO: 388 - protein, 120 aa, synthetic construct
QVQLQESGGGMVHPGGSLRLSCAASGFTFSGSDMSWVRQAPGKGPEWVSYITSGGGTYYPDSVKGRFTISRDNAKNTLYLQMNSLKPEDTALYRCAKARLTDSHYVEDAWGQGTQVTVSS

SEQ ID NO: 389 - protein, 121 aa, synthetic construct
QVQLQESGGGLVQPGGSLRLSCAASGFTFSSYDMSWVRQAPGKGPEWVSTINTGGGSTYYADSVKGRFTISRDNAKNTLYLQMNSLKPDDTALYRCAKARLTDSHYVEDAWGQGTQVTVSS

SEQ ID NO: 390 - protein, 120 aa, synthetic construct
QVQLQESGGGMVHPGGSLRLSCAASGFTFSGSDMSWVRQAPGKGPEWVSYITGGGGTYYADSVKGRFTISRDNAKNTLYLQMNSLKPEDTALYRCAKARLTDSHYVEDAWGQGTQVTVSS

SEQ ID NO: 391 - protein, 5 aa, synthetic construct
GSDMS

SEQ ID NO: 392 - protein, 16 aa, synthetic construct
YITAGGSTEYADSVKG

SEQ ID NO: 393 - protein, 12 aa, synthetic construct
ARLTDSHYVEDA

SEQ ID NO: 394 - protein, 8 aa, synthetic construct
GFTFSGSD

SEQ ID NO: 395 - protein, 7 aa, synthetic construct
ITAGGST

SEQ ID NO: 396 - protein, 14 aa, synthetic construct
AKARLTDSHYVEDA

SEQ ID NO: 397 - protein, 120 aa, synthetic construct
QVQLQESGGGMVLPGGSLRLSCAASGFTFSGSDMSWVRQAPGKGPEWVSYITAGGSTEYADSVKGRFTISRDNAKKKLYLQMNSLKPEDTALYRCAKARLTDSHYVEDAWGQGTQVTVSS

SEQ ID NO: 398 - protein, 120 aa, synthetic construct
QVQLQESGGGLVQPGGSLRLSCAASGFTFSGSDMSWVRQAPGKGPEWVSYITAGGSTEYADSVKGRFTISRDNAKKKLYLQMNSLKPEDTALYRCAKARLTDSHYVEDAWGQGTQVTVSS

SEQ ID NO: 399 - protein, 5 aa, synthetic construct
GSDMS

SEQ ID NO: 400 - protein, 16 aa, synthetic construct
YITGGGTTYYPDSVKG

SEQ ID NO: 401 - protein, 12 aa, synthetic construct
ARLTDSHYVEDA

SEQ ID NO: 402 - protein, 8 aa, synthetic construct
GFAFSGSD

SEQ ID NO: 403 - protein, 7 aa, synthetic construct
ITGGGTT

SEQ ID NO: 404 - protein, 14 aa, synthetic construct
AKARLTDSHYVEDA

SEQ ID NO: 405 - protein, 120 aa, synthetic construct
QVQLQESGGGMVHPGGSLRLSCAASGFAFSGSDMSWVRQAPGKGPEWVSYITGGGTTYYPDSVKGRFTISRDNAKNTLYLQMNSLKPDDTALYRCAKARLTDSHYVEDAWGQGTQVTVSS

SEQ ID NO: 406 - protein, 5 aa, synthetic construct
GSDMS

SEQ ID NO: 407 - protein, 16 aa, synthetic construct
YITGGGSTYYADSVKG

SEQ ID NO: 408 - protein, 12 aa, synthetic construct
ARLTDSHYVEDA

SEQ ID NO: 409 - protein, 8 aa, synthetic construct
GFTFSGSD

SEQ ID NO: 410 - protein, 7 aa, synthetic construct
ITGGGST

SEQ ID NO: 411 - protein, 14 aa, synthetic construct
AKARLTDSHYVEDA

SEQ ID NO: 412 - protein, 120 aa, synthetic construct
QVQLQESGGGMVHPGGSLRLSCASSGFTFSGSDMSWVRQAPGKGPEWVSYITGGGSTYYADSVKGRFTISRDNAKNTLYLQMNNLKPEDTALYRCAKARLTDSHYVEDAWGQGTQVTVSS

SEQ ID NO: 413 - protein, 5 aa, synthetic construct
GSDMS

SEQ ID NO: 414 - protein, 16 aa, synthetic construct
YITAGGTTYYPDSVKG

SEQ ID NO: 415 - protein, 12 aa, synthetic construct
ARLTDSHYVEDA

SEQ ID NO: 416 - protein, 8 aa, synthetic construct
GRTFSGSD

SEQ ID NO: 417 - protein, 7 aa, synthetic construct
ITAGGTT

SEQ ID NO: 418 - protein, 14 aa, synthetic construct
AKARLTDSHYVEDA

SEQ ID NO: 419 - protein, 120 aa, synthetic construct
QVQLQESGGGLVQAGGSLRLSCAASGRTFSGSDMSWVRQAPGKGPEWVSYITAGGTTYYPDSVKGRFTISRDNAKNTLYLQMNSLKPDDTALYRCAKARLTDSHYVEDAWGQGTQVTVSS

SEQ ID NO: 420 - protein, 5 aa, synthetic construct
GSDMS

SEQ ID NO: 421 - protein, 16 aa, synthetic construct
YITSGGTTYYPDSVKG

SEQ ID NO: 422 - protein, 12 aa, synthetic construct
VRLTDSHYVEDA

SEQ ID NO: 423 - protein, 8 aa, synthetic construct
GFTFSGSD

SEQ ID NO: 424 - protein, 7 aa, synthetic construct
ITSGGTT

SEQ ID NO: 425 - protein, 14 aa, synthetic construct
AKVRLTDSHYVEDA

SEQ ID NO: 426 - protein, 120 aa, synthetic construct
QVQLQESGGGMVHPGGSLRLSCAASGFTFSGSDMSWVRQAPGKGPEWVSYITSGGTTYYPDSVKGRFTISRDNAKNTLYLQMNSLKPDDTALYRCAKVRLTDSHYVEDAWGQGTQVTVSS

SEQ ID NO: 427 - protein, 5 aa, synthetic construct
GSDMS

SEQ ID NO: 428 - protein, 16 aa, synthetic construct
YITGGGTTYYPDSVKG

SEQ ID NO: 429 - protein, 12 aa, synthetic construct
ARLTDGHYVEDA

SEQ ID NO: 430 - protein, 8 aa, synthetic construct
GFTFSGSD

SEQ ID NO: 431 - protein, 7 aa, synthetic construct
ITGGGTT

SEQ ID NO: 432 - protein, 14 aa, synthetic construct
AKARLTDGHYVEDA

SEQ ID NO: 433 - protein, 120 aa, synthetic construct
QVQLQESGGGMVHPGGSLRLSCAASGFTFSGSDMSWVRQAPGKGPEWVSYITGGGTTYYPDSVKGRFTISRDNAKNTLYLQMNSLKPDDTALYRCAKARLTDGHYVEDAWGQGTQVTVSS

SEQ ID NO: 434 - protein, 5 aa, synthetic construct
GSDMS

SEQ ID NO: 435 - protein, 16 aa, synthetic construct
YITGGGTTYYPDSVKG

SEQ ID NO: 436 - protein, 12 aa, synthetic construct
ARLTDSHYVEDA

SEQ ID NO: 437 - protein, 8 aa, synthetic construct
GLTFSGSD

SEQ ID NO: 438 - protein, 7 aa, synthetic construct
ITGGGTT

SEQ ID NO: 439 - protein, 14 aa, synthetic construct
AKARLTDSHYVEDA

SEQ ID NO: 440 - protein, 120 aa, synthetic construct
QVQLQESGGGLVQPGGSLRLSCAASGLTFSGSDMSWVRQAPGKGPEWVSYITGGGTTYYPDSVKGRFTISRDNAKNTLYLQMNSLKPDDTALYRCAKARLTDSHYVEDAQGQGTQVTVSS

SEQ ID NO: 441 - protein, 5 aa, synthetic construct
SADMS

SEQ ID NO: 442 - protein, 17 aa, synthetic construct
YINTGGSSTYYADSVKG

SEQ ID NO: 443 - protein, 12 aa, synthetic construct
ARLTDSHYVEDY

SEQ ID NO: 444 - protein, 8 aa, synthetic construct
GFTFSSAD

SEQ ID NO: 445 - protein, 8 aa, synthetic construct
INTGGSST

SEQ ID NO: 446 - protein, 14 aa, synthetic construct
AKARLTDSHYVEDY

SEQ ID NO: 447 - protein, 121 aa, synthetic construct
QVQLQESGGGLVQPGGSLRLSCAASGFTFSSADMSWVRQAPGKGPEWVSYINTGGSSTYYADSVKGRFTISRDDAKSTLYLQMNSLKPEDTALYRCAKARLTDSHYVEDYWGQGTQVTVSS

SEQ ID NO: 448 - protein, 5 aa, synthetic construct
SYDMS

SEQ ID NO: 449 - protein, 17 aa, synthetic construct
TINTGGGTTYYADSVKG

SEQ ID NO: 450 - protein, 12 aa, synthetic construct
ARLTDSHYVEDA

SEQ ID NO: 451 - protein, 8 aa, synthetic construct
GFTFSSYD

SEQ ID NO: 452 - protein, 8 aa, synthetic construct
INTGGGTT

SEQ ID NO: 453 - protein, 14 aa, synthetic construct
AKARLTDSHYVEDA

SEQ ID NO: 454 - protein, 121 aa, synthetic construct
QVQLQESGGGMVQPGGSLRLSCAASGFTFSSYDMSWVRQAPGKGPEWVSTINTGGGTTYYADSVKGRFTISRDNAKNTLYLQMNSLKPDDTALYRCAKARLTDSHYVEDAWGQGTQVTVSS

SEQ ID NO: 455 - protein, 5 aa, synthetic construct
SNDMS

SEQ ID NO: 456 - protein, 17 aa, synthetic construct
TIKGDGGATYYADSVKG

SEQ ID NO: 457 - protein, 12 aa, synthetic construct
ARLTDSHYVEDA

SEQ ID NO: 458 - protein, 8 aa, synthetic construct
GFTFSSND

SEQ ID NO: 459 - protein, 8 aa, synthetic construct
IKGDGGAT

SEQ ID NO: 460 - protein, 14 aa, synthetic construct
AKARLTDSHYVEDA

SEQ ID NO: 461 - protein, 121 aa, synthetic construct
QVQLQESGGGMVQPGGSLRLSCAASGFTFSSNDMSWVRQAPGKGPEWVSTIKGDGGATYYADSVKGRFTISRDNAKNTLYLQMNSLKPDDTALYRCAKARLTDSHYVEDAWGQGTQVTVSS

SEQ ID NO: 462 - protein, 5 aa, synthetic construct
DSDMS

SEQ ID NO: 463 - protein, 16 aa, synthetic construct
YITAGGITYYADSVKG

SEQ ID NO: 464 - protein, 12 aa, synthetic construct
ARLTETHYVEDA

SEQ ID NO: 465 - protein, 8 aa, synthetic construct
GFTFSDSD

SEQ ID NO: 466 - protein, 7 aa, synthetic construct
ITAGGIT

SEQ ID NO: 467 - protein, 14 aa, synthetic construct
AKARLTETHYVEDA

SEQ ID NO: 468 - protein, 120 aa, synthetic construct
QVQLQESGGGMVHPGGSLRLSCAASGFTFSDSDMSWVRQAPGKGPEWVSYITAGGITYYADSVKGRFTISRDNADHTLYLQMNSLKPEDTALYRCAKARLTETHYVEDAWGQGTQVTVSS

SEQ ID NO: 469 - protein, 120 aa, synthetic construct
EVQLLESGGGMVQPGGSLRLSCAASGFTFSGSDMSWVRQAPGKGLEWVSYITSGGTTYYADTVKGRFTISRDNSKNTLYLQMNSLRAEDTAVYRCAKARLTDSHYVEDAWGQGTLVTVSS

SEQ ID NO: 470 - protein, 120 aa, synthetic construct
EVQLLESGGGLVQPGGSLRLSCAASGFTFSGSDMSWVRQAPGKGLEWVSYITSGGTTYYADTVKGRFTISRDNSKNTLYLQMNSLRAEDTAVYRCAKARLTDSHYVEDAWGQGTLVTVSS

SEQ ID NO: 471 - protein, 120 aa, synthetic construct
QVQLQESGGGMIQPGGSLRLSCAASGFTFSGSDMSWVRQAPGKGPEWVSYITSGGTTYYPDTVKGRFTISRDNSKNTLYLQMNSLRADDTALYRCAKARLTDSHYVEDAWGQGTLVTVSS

SEQ ID NO: 472 - protein, 120 aa, synthetic construct
QVQLQESGGGLIQPGGSLRLSCAASGFTFSGSDMSWVRQAPGKGPEWVSYITSGGTTYYPDTVKGRFTISRDNSKNTLYLQMNSLRADDTALYRCAKARLTDSHYVEDAWGQGTLVTVSS

SEQ ID NO: 473 - protein, 120 aa, synthetic construct
QVQLQESGGGLVHPGGSLRLSCAASGFTFSGSDMSWVRQAPGKGPEWVSYITSGGTTYYPDTVKGRFTISRDNAKNTLYLQMNSLTPEDTALYRCAKARLTDSHYVEDAWGQGTQVTVSS

SEQ ID NO: 474 - protein, 94 aa, synthetic construct
  MOTIF 26: note = X is an amino acid sequence of IMGT CDR1
  MOTIF 44: note = X is an amino acid sequenceof IMGT CDR2
  MOTIF 83: note = X is an amino acid sequence of IMGT CDR3
EVQLLESGGGMVQPGGSLRLSCAASXMSWVRQAPGKGLEWVSYXYYADTVKGRFTISRDNSKNTLYLQMNSLRAEDTAVYRCXWGQGTLVTVSS

SEQ ID NO: 475 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequenceof IMGT CDR3
QVQLQESGGGMIQPGGSLRLSCAASXMSWVRQAPGKGPEWVSYXYYPDTVKGRFTISRDNSKNTLYLQMNSLRADDTALYRCXWGQGTLVTVSS

SEQ ID NO: 476 - protein, 94 aa, synthetic construct
  MOTIF 26: note = X is an amino acid sequence of IMGT CDR1
  MOTIF 44: note = X is an amino acid sequence of IMGT CDR2
  MOTIF 83: note = X is an amino acid sequence of IMGT CDR3
QVQLQESGGGLVHPGGSLRLSCAASXMSWVRQAPGKGPEWVSYXYYPDTVKGRFTISRDNAKNTLYLQMNSLTPEDTALYRCXWGQGTQVTVSS

SEQ ID NO: 477 - protein, 94 aa, synthetic construct; identical to SEQ ID NO: 211
  VARIANT 1: note = Xaa is Q or E
  VARIANT 5: note = Xaa is Q, V or L
  VARIANT 11: note = Xaa is M or L
  VARIANT 14: note = Xaa is V or P
  MOTIF 26: note = X is an amino acid sequence of IMGT CDR1
  VARIANT 37: note = Xaa is G or Q
  VARIANT 38: note = Xaa is P, L or R
  VARIANT 43: note = Xaa is Y or V
  MOTIF 44: note = X is an amino acid sequence of IMGT CDR2
  VARIANT 47: note = Xaa is P or A
  VARIANT 49: note = Xaa is S or T
  VARIANT 61: note = Xaa is A or S
  VARIANT 68: note = Xaa is L or Q
  VARIANT 71: note = Xaa is N or S
  VARIANT 73: note = Xaa is K or R
  VARIANT 74: note = Xaa is P or A
  VARIANT 79: note = Xaa is L or V
  VARIANT 81: note = Xaa is R or Y
  MOTIF 83: note = X is an amino acid sequence of IMGT CDR3
  VARIANT 89: note = Xaa is Q or M

SEQ ID NO: 478 - protein, 94 aa, synthetic construct; identical to SEQ ID NO: 211
  VARIANT 1: note = Xaa is Q or E
  VARIANT 5: note = Xaa is Q, V or L
  VARIANT 11: note = Xaa is M or L
  VARIANT 14: note = Xaa is V or P
  MOTIF 26: note = X is an amino acid sequence of IMGT CDR1
  VARIANT 37: note = Xaa is G or Q
  VARIANT 38: note = Xaa is P, L or R
  VARIANT 43: note = Xaa is Y
  MOTIF 44: note = X is an amino acid sequence of IMGT CDR2
  VARIANT 47: note = Xaa is P or A
  VARIANT 49: note = Xaa is S or T
  VARIANT 61: note = Xaa is A or S
  VARIANT 68: note = Xaa is L or Q
  VARIANT 71: note = Xaa is N or S
  VARIANT 73: note = Xaa is K or R
  VARIANT 74: note = Xaa is P or A
  VARIANT 79: note = Xaa is L or V
  MOTIF 83: note = X is an amino acid sequence of IMGT CDR3
  VARIANT 89: note = Xaa is Q or M

SEQ ID NO: 479 - protein, 94 aa, synthetic construct; identical to SEQ ID NO: 213
  VARIANT 1: note = Xaa is Q or E
  VARIANT 5: note = Xaa is Q, L or V
  VARIANT 11: note = Xaa is L
  VARIANT 12: note = Xaa is V or I
  VARIANT 13: note = Xaa is E, D, Q or H
  VARIANT 14: note = Xaa is A or P
  VARIANT 23: note = Xaa is A
  MOTIF 26: note = X is an amino acid sequence of IMGT CDR1
  VARIANT 38: note = Xaa is P or L
  VARIANT 43: note = Xaa is Y, A or V
  MOTIF 44: note = X is an amino acid sequence of IMGT CDR2
  VARIANT 47: note = Xaa is P or A
  VARIANT 49: note = Xaa is S or T
  VARIANT 61: note = Xaa is A or S
  VARIANT 73: note = Xaa is K, R or T
  VARIANT 74: note = Xaa is P or A
  VARIANT 75: note = Xaa is D or E
  VARIANT 79: note = Xaa is L or V
  VARIANT 81: note = Xaa is R or Y
  MOTIF 83: note = X is an amino acid sequence of IMGT CDR3
  VARIANT 84: note = Xaa is W
  VARIANT 89: note = Xaa is Q or L

SEQ ID NO: 480 - protein, 94 aa, synthetic construct; identical to SEQ ID NO: 213
  VARIANT 1: note = Xaa is Q or E
  VARIANT 5: note = Xaa is Q, L or V
  VARIANT 11: note = Xaa is L or M
  VARIANT 12: note = Xaa is V or I
  VARIANT 13: note = Xaa is E, D, Q or H
  VARIANT 14: note = Xaa is A or P
  VARIANT 23: note = Xaa is A or V
  MOTIF 26: note = X is an amino acid sequence of IMGT CDR1
  VARIANT 38: note = Xaa is P or L
  VARIANT 43: note = Xaa is Y, A, V or I
  MOTIF 44: note = X is an amino acid sequence of IMGT CDR2
  VARIANT 47: note = Xaa is P or A
  VARIANT 49: note = Xaa is S or T
  VARIANT 61: note = Xaa is A or S
  VARIANT 73: note = Xaa is K, R or T
  VARIANT 74: note = Xaa is P or A
  VARIANT 75: note = Xaa is D or E
  VARIANT 79: note = Xaa is L or V
  VARIANT 81: note = Xaa is R or Y
  MOTIF 83: note = X is an amino acid sequence of IMGT CDR3
  VARIANT 84: note = Xaa is W or Q
  VARIANT 89: note = Xaa is Q or L

SEQ ID NO: 481 - protein, 15 aa, synthetic construct
EPKSSDKTHTCPPCP

SEQ ID NO: 482 - protein, 352 aa, synthetic construct
EVQLLESGGGLVQPGGSLRLSCAASGFTFSGSDMSWVRQAPGKGLEWVSYITSGGTTYYADTVKGRFTISRDNSKNTLYLQMNSLRAEDTAVYRCAKARLTDSHYVEDAWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 483 - protein, 352 aa, synthetic construct
QVQLQESGGGLIQPGGSLRLSCAASGFTFSGSDMSWVRQAPGKGPEWVSYITSGGTTYYPDTVKGRFTISRDNSKNTLYLQMNSLRADDTALYRCAKARLTDSHYVEDAWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 484 - protein, 352 aa, synthetic construct
QVQLQESGGGLVHPGGSLRLSCAASGFTFSGSDMSWVRQAPGKGPEWVSYITSGGTTYYPDTVKGRFTISRDNAKNTLYLQMNSLTPEDTALYRCAKARLTDSHYVEDAWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 485 - protein, 5 aa, synthetic construct
GSDMS

SEQ ID NO: 486 - protein, 16 aa, synthetic construct
YITGSGTTYYPDSVKG

SEQ ID NO: 487 - protein, 12 aa, synthetic construct
ARLTDSHYVEDA

SEQ ID NO: 488 - protein, 8 aa, synthetic construct
GFTLSGSD

SEQ ID NO: 489 - protein, 7 aa, synthetic construct
ITGSGTT

SEQ ID NO: 490 - protein, 7 aa, synthetic construct
ITGSGTT

SEQ ID NO: 491 - protein, 120 aa, synthetic construct
QVQLQESGGGLVQPGRSLRLSCAASGFTLSGSDMSWVRQAPGKWPEWVSYITGSGTTYYPDSVKGRFTTSRDNAKNMLYLQMNSLKPDDTALYRCAKARLTDSHYVEDAWGQGTQVTVSS

SEQ ID NO: 492 - protein, 16 aa, synthetic construct
YITGSGTTYYADTVKG

SEQ ID NO: 493 - protein, 120 aa, synthetic construct
EVQLLESGGGLVQPGGSLRLSCAASGFTLSGSDMSWVRQAPGKWPEWVSYITGSGTTYYADTVKGRFTTSRDNAKNTLYLQMNSLRAEDTAVYRCAKARLTDSHYVEDAWGQGTLVTVSS

SEQ ID NO: 494 - protein, 352 aa, synthetic construct
EVQLLESGGGLVQPGGSLRLSCAASGFTLSGSDMSWVRQAPGKWPEWVSYITGSGTTYYADTVKGRFTTSRDNAKNTLYLQMNSLRAEDTAVYRCAKARLTDSHYVEDAWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 495 - protein, 16 aa, synthetic construct
YITGSGTTYYADSVKG

SEQ ID NO: 496 - protein, 120 aa, synthetic construct
EVQLLESGGGLVQPGRSLRLSCAASGFTLSGSDMSWVRQAPGKWPEWVSYITGSGTTYYADSVKGRFTTSRDNAKNTLYLQMNSLRAEDTAVYRCAKARLTDSHYVEDAWGQGTLVTVSS

SEQ ID NO: 497 - protein, 352 aa, synthetic construct
EVQLLESGGGLVQPGRSLRLSCAASGFTLSGSDMSWVRQAPGKWPEWVSYITGSGTTYYADSVKGRFTTSRDNAKNTLYLQMNSLRAEDTAVYRCAKARLTDSHYVEDAWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 498 - protein, 16 aa, synthetic construct
YITGSGTTYYPDSAKG

SEQ ID NO: 499 - protein, 12 aa, synthetic construct
ARLTDGHYVEDA

SEQ ID NO: 500 - protein, 8 aa, synthetic construct
GFTFSGSD

SEQ ID NO: 501 - protein, 14 aa, synthetic construct
AKARLTDGHYVEDA

SEQ ID NO: 502 - protein, 120 aa, synthetic construct
QVQLQESGGGLVQPGRSLRLSCAASGFTFSGSDMSWVRQAPGKGPEWVSYITGSGTTYYPDSAKGRFTTSRDNAKNMLYLQMNNLKPDDTALYRCAKARLTDGHYVEDAWGQGTQVTVSS

SEQ ID NO: 503 - protein, 120 aa, synthetic construct
EVQLVESGGGLVQPGRSLRLSCAASGFTFSGSDMSWVRQAPGKGPEWVSYITGSGTTYYPDSVKGRFTISRDNSKNTLYLQMNSLRAEDTALYRCAKARLTDGHYVEDAWGQGTLVTVSS

SEQ ID NO: 504 - protein, 352 aa, synthetic construct
EVQLVESGGGLVQPGRSLRLSCAASGFTFSGSDMSWVRQAPGKGPEWVSYITGSGTTYYPDSVKGRFTISRDNSKNTLYLQMNSLRAEDTALYRCAKARLTDGHYVEDAWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 505 - protein, 17 aa, synthetic construct
MSWVRQAPGKWPEWVSY

SEQ ID NO: 506 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 507 - protein, 120 aa, synthetic construct
  VARIANT 1..115: note = Xaa is any amino acid residue
XVQLXESGGGXXXXGXSLRLSCXASGFTFSGSDMSWVRQAPGKGXEWVSYITSGGTTYYXDXVKGRFTISRDNXXNXLYLQMNSLXXXDTAXXRCAKARLTDSHYVEDAXGQGTXVTVSS

SEQ ID NO: 508 - protein, 120 aa, synthetic construct; identical to SEQ ID NO: 507
  VARIANT 1: note = Xaa is E, Q, N, K or D
  VARIANT 5: note = Xaa is any amino acid residue
  VARIANT 11: note = Xaa is L, M, I or V
  VARIANT 12: note = Xaa is I, V, L or M
  VARIANT 13: note = Xaa is Q, H or D
  VARIANT 14: note = Xaa is any amino acid residue
  VARIANT 16: note = Xaa is G, D, S or N
  VARIANT 23: note = Xaa is A, V or T
  VARIANT 45: note = Xaa is any amino acid residue
  VARIANT 60: note = Xaa is any amino acid residue
  VARIANT 62: note = Xaa is S, T or A
  VARIANT 74: note = Xaa is A, S or T
  VARIANT 75: note = Xaa is K, N, E, Q, D or H
  VARIANT 77: note = Xaa is any amino acid residue
  VARIANT 86: note = Xaa is any amino acid residue
  VARIANT 87: note = Xaa is any amino acid residue
  VARIANT 88: note = Xaa is D, E, N or Q
  VARIANT 92: note = Xaa is L, V, M or I
  VARIANT 93: note = Xaa is any amino acid residue
  VARIANT 110: note = Xaa is any amino acid residue
  VARIANT 115: note = Xaa is any amino acid residue

SEQ ID NO: 509 - protein, 120 aa, synthetic construct; identical to SEQ ID NO: 507
  VARIANT 1: note = Xaa is E or Q
  VARIANT 5: note = Xaa is Q or L
  VARIANT 11: note = Xaa is L or M
  VARIANT 12: note = Xaa is I or V
  VARIANT 13: note = Xaa is Q, H or D
  VARIANT 14: note = Xaa is P or A
  VARIANT 16: note = Xaa is G or D
  VARIANT 23: note = Xaa is A or V
  VARIANT 45: note = Xaa is P or L
  VARIANT 60: note = Xaa is P or A
  VARIANT 62: note = Xaa is S or T
  VARIANT 74: note = Xaa is A or S
  VARIANT 75: note = Xaa is K or N
  VARIANT 77: note = Xaa is T or M
  VARIANT 86: note = Xaa is R, K or T
  VARIANT 87: note = Xaa is P or A
  VARIANT 88: note = Xaa is D or E
  VARIANT 92: note = Xaa is L or V
  VARIANT 93: note = Xaa is Y or D
  VARIANT 110: note = Xaa is W or Q
  VARIANT 115: note = Xaa is L or Q

SEQ ID NO: 510 - protein, 120 aa, synthetic construct; identical to SEQ ID NO: 507
  VARIANT 11: note = Xaa is M or L
  VARIANT 13: note = Xaa is H, D or Q
  VARIANT 14: note = Xaa is P or A
  VARIANT 16: note = Xaa is G or D
  VARIANT 23: note = Xaa is A or V
  VARIANT 75: note = Xaa is K or N
  VARIANT 77: note = Xaa is T or M
  VARIANT 86: note = Xaa is K or T
  VARIANT 88: note = Xaa is D or E
  VARIANT 93: note = Xaa is Y or D
  VARIANT 110: note = Xaa is W or Q

SEQ ID NO: 511 - protein, 120 aa, synthetic construct; identical to SEQ ID NO: 507
  VARIANT 1: note = Xaa is E or Q
  VARIANT 5: note = Xaa is Q or L
  VARIANT 11: note = Xaa is L or M
  VARIANT 12: note = Xaa is I or V
  VARIANT 13: note = Xaa is Q or H
  VARIANT 45: note = Xaa is P or L
  VARIANT 60: note = Xaa is P or A
  VARIANT 62: note = xaa is S or T
  VARIANT 74: note = Xaa is A or S
  VARIANT 86: note = Xaa is R or T
  VARIANT 87: note = Xaa is A or P
  VARIANT 88: note = Xaa is D or E
  VARIANT 92: note = Xaa is L or V
  VARIANT 115: note = Xaa is L or Q

SEQ ID NO: 512 - protein, 120 aa, synthetic construct
  VARIANT 16: note = Xaa is any amino acid residue
  VARIANT 69: note = Xaa is any amino acid residue
  VARIANT 77: note = Xaa is any amino acid residue
QVQLQESGGGLVQPGXSLRLSCAASGFTFSGSDMSWVRQAPGKGPEWVSYITGSGTTYYPDSVKGRFTXSRDNAKNXLYLQMNSLKPDDTALYRCAKARLTDSHYVEDAWGQGTQVTVSS

SEQ ID NO: 513 - protein, 118 aa, synthetic construct
  VARIANT 16: note = Xaa is R or G
  VARIANT 69: note = Xaa is T or I
  VARIANT 77: note = Xaa is M or T
QVQLQESGGGLVQPGSLRLSCAASGFTFSGSDMSWVRQAPGKGPEWVSYITGSGTTYYPDSVKGRFTSRDNAKNXLYLQMNSLKPDDTALYRCAKARLTDSHYVEDAWGQGTQVTVSS

SEQ ID NO: 514 - protein, 120 aa, synthetic construct
  VARIANT 11..110: note = Xaa is any amino acid residue
QVQLQESGGGXVXXGGSLRLSCXASGFTFSGSDMSWVRQAPGKGPEWXSYITGGGTTYYPDSVKGRFTISRDNAXNXLYLQMNSXKPXDTAXYRCAKARLTDSHYVEDAXGQGTQVTVSS

SEQ ID NO: 515 - protein, 120 aa, synthetic construct; identical to SEQ ID NO: 514
  VARIANT 11: note = Xaa is M, L, I or V
  VARIANT 13: note = Xaa is H, Q, N, K or R
  VARIANT 14: note = Xaa is any amino acid residue
  VARIANT 23: note = Xaa is A, V or T
  VARIANT 48: note = Xaa is any amino acid residue
  VARIANT 75: note = Xaa is K, N, E, Q or H
  VARIANT 77: note = Xaa is any amino acid residue
  VARIANT 85: note = Xaa is any amino acid residue
  VARIANT 88: note = Xaa is E, D, N or Q
  VARIANT 92: note = Xaa is L, V, M or I
  VARIANT 110: note = Xaa is any amino acid residue

SEQ ID NO: 516 - protein, 120 aa, synthetic construct; identical to SEQ ID NO: 514
  VARIANT 11: note = Xaa is M or L
  VARIANT 13: note = Xaa is H or Q
  VARIANT 14: note = Xaa is P or A
  VARIANT 23: note = Xaa is A or V
  VARIANT 48: note = Xaa is V or G
  VARIANT 75: note = Xaa is K or N
  VARIANT 77: note = Xaa is T or M
  VARIANT 85: note = Xaa is R or L
  VARIANT 88: note = Xaa is E or D
  VARIANT 92: note = Xaa is L or V
  VARIANT 110: note = Xaa is W or Q

SEQ ID NO: 517 - protein, 120 aa, synthetic construct
  VARIANT 11..110: note = Xaa is any amino acid residue
QVQLQESGGGXVXXGGSLRLSCXASGFTFSGSDMSWVRQAPGKGPEWVSYITAGGTTYYPDSVKGRFTISRDNAKNTLYLQMNSLKPDDTALYRCAKARLTDSHYVEDAXGQGTQVTVSS

SEQ ID NO: 518 - protein, 120 aa, synthetic construct; identical to SEQ ID NO: 517
  VARIANT 11: note = Xaa is L, M, I or V
  VARIANT 13: note = Xaa is Q, H, N or K
  VARIANT 14: note = Xaa is any amino acid residues
  VARIANT 23: note = Xaa is A, V or T
  VARIANT 110: note = Xaa is any amino acid residue

SEQ ID NO: 519 - protein, 120 aa, synthetic construct; identical to SEQ ID NO: 517
  VARIANT 11: note = Xaa is L or M
  VARIANT 13: note = Xaa is Q or H
  VARIANT 14: note = Xaa is A or P
  VARIANT 23: note = Xaa is A or V
  VARIANT 110: note = Xaa is W or Q

SEQ ID NO: 520 - protein, 120 aa, synthetic construct
  VARIANT 11: note = Xaa is any amino acid residue
  VARIANT 14: note = Xaa is any amino acid residue
QVQLQESGGGXVXPGGSLRLSCAASGFTFSGSDMSWVRQAPGKGPEWVSYITSGGGTYYPDSVKGRFTISRDNAKNTLYLQMNSLKPEDTALYRCAKARLTDSHYVEDAWGQGTQVTVSS

SEQ ID NO: 521 - protein, 120 aa, synthetic construct; identical to SEQ ID NO: 520
  VARIANT 11: note = Xaa is M, L, I or V
  VARIANT 14: note = Xaa is H, Q, N or K

SEQ ID NO: 522 - protein, 120 aa, synthetic construct; identical to SEQ ID NO: 520
  VARIANT 11: note = xaa is M or L
  VARIANT 14: note = Xaa is H or Q

SEQ ID NO: 523 - protein, 120 aa, synthetic construct
  VARIANT 73: note = Xaa is any amino acid residue
  VARIANT 77: note = Xaa is any amino acid residue
QVQLQESGGGMVHPGGSLRLSCAASGFTFSGSDMSWVRQAPGKGPEWVSYITGGGGTYYADSVKGRFTISRDXAKNXLYLQMNSLKPEDTALYRCAKARLTDSHYVEDAWGQGTQVTVSS

SEQ ID NO: 524 - protein, 120 aa, synthetic construct; identical to SEQ ID NO: 523
  VARIANT 73: note = Xaa is N, H, Q or K
  VARIANT 77: note = Xaa is any amino acid residue

SEQ ID NO: 525 - protein, 120 aa, synthetic construct; identical to SEQ ID NO: 523
  VARIANT 73: note = Xaa is N or H
  VARIANT 77: note = Xaa is M or T

SEQ ID NO: 526 - protein, 120 aa, synthetic construct
  VARIANT 11: note = Xaa is any amino acid residue
  VARIANT 13: note = Xaa is any amino acid residue
QVQLQESGGGXVXPGGSLRLSCAASGFTFSGSDMSWVRQAPGKGPEWVSYITAGGSTEYADSVKGRFTISRDNAKKKLYLQMNSLKPEDTALYRCAKARLTDSHYVEDAWGQGTQVTVSS

SEQ ID NO: 527 - protein, 120 aa, synthetic construct; identical to SEQ ID NO: 526
  VARIANT 11: note = Xaa is M, L, I or V
  VARIANT 13: note = Xaa is any amino acid residue

SEQ ID NO: 528 - protein, 120 aa, synthetic construct; identical to SEQ ID NO: 526
  VARIANT 11: note = Xaa is M or L
  VARIANT 13: note = Xaa is Q or L

SEQ ID NO: 529 - protein, 121 aa, synthetic construct
  VARIANT 11..89: note = Xaa is any amino acid residue
QVQLQESGGGXVQPGGSLRLSCAASGFTFSSYDMSWVRQAPGKGPEWVSTINTGGGSTYYADSVKGRFTISRDNAXNXLYLQMNSLKPXDTALYRCAKARLTDSHYVEDAWGQGTQVTVSS

SEQ ID NO: 530 - protein, 121 aa, synthetic construct; identical to SEQ ID NO: 529
  VARIANT 11: note = Xaa is M, L, I or V
  VARIANT 76: note = Xaa is N, K, E, Q or D
  VARIANT 78: note = Xaa is any amino acid residue
  VARIANT 89: note = Xaa is D, E, N or Q

SEQ ID NO: 531 - protein, 121 aa, synthetic construct; identical to SEQ ID NO: 529
  VARIANT 11: note = Xaa is M or L
  VARIANT 76: note = Xaa is N or K
  VARIANT 78: note = Xaa is M or T
  VARIANT 89: note = Xaa is D or E

SEQ ID NO: 532 - protein, 5 aa, synthetic construct
RSSMS

SEQ ID NO: 533 - protein, 17 aa, synthetic construct
IISSSGIITHYSDSVKG

SEQ ID NO: 534 - protein, 9 aa, synthetic construct
VLDDDSGDY

SEQ ID NO: 535 - protein, 8 aa, synthetic construct
GFTFSRSS

SEQ ID NO: 536 - protein, 8 aa, synthetic construct
ISSSGIIT

SEQ ID NO: 537 - protein, 11 aa, synthetic construct
NAVLDDDSGDY

SEQ ID NO: 538 - protein, 118 aa, synthetic construct
QVQLQESGGGWVQPGGSLRLSCVASGFTFSRSSMSWHRQAPGKERDLVAIISSSGIITHYSDSVKGRFTISRDNAKDTVYLQMNSLKPEDTAVYVCNAVLDDDSGDYWGQGTQVTVSS

SEQ ID NO: 539 - protein, 120 aa, synthetic construct
  VARIANT 11..48: note = Xaa is any amino acid residue
  VARIANT 53: note = Xaa is A or G
  VARIANT 75..110: note = Xaa is any amino acid residue
QVQLQESGGGXVXXGGSLRLSCXASGFTFSGSDMSWVRQAPGKGPEWXSYITXGGTTYYPDSVKGRFTISRDNAXNXLYLQMNSXKPXDTAXYRCAKARLTDSHYVEDAXGQGTQVTVSS

SEQ ID NO: 540 - protein, 120 aa, synthetic construct; identical to SEQ ID NO: 539
  VARIANT 11: note = Xaa is M, L, I or V
  VARIANT 13: note = Xaa is H, Q, N, K or R
  VARIANT 14: note = Xaa is any amino acid residue
  VARIANT 23: note = Xaa is A, V or T
  VARIANT 48: note = Xaa is any amino acid residue
  VARIANT 53: note = Xaa is A or G
  VARIANT 75: note = Xaa is K, N, E, Q or H
  VARIANT 77: note = Xaa is any amino acid residue
  VARIANT 85: note = Xaa is any amino acid residue
  VARIANT 88: note = Xaa is E, D, N or Q
  VARIANT 92: note = Xaa is L, V, M or I
  VARIANT 110: note = Xaa is any amino acid residue

SEQ ID NO: 541 - protein, 120 aa, synthetic construct; identical to SEQ ID NO: 539
  VARIANT 11: note = Xaa is M or L
  VARIANT 13: note = Xaa is H or Q
  VARIANT 14: note = Xaa is P or A
  VARIANT 23: note = Xaa is A or V
  VARIANT 48: note = Xaa is V or G
  VARIANT 53: note = Xaa is A or G
  VARIANT 75: note = Xaa is K or N
  VARIANT 77: note = Xaa is T or M
  VARIANT 85: note = Xaa is R or L
  VARIANT 88: note = Xaa is E or D
  VARIANT 92: note = Xaa is L or V
  VARIANT 110: note = Xaa is W or Q

(19) United States
(12) Patent Application Publication
(10) Pub. No.: US 2026/0297210 A1
(43) Pub. Date: Oct. 1, 2026

(54) BINDING AGENTS TARGETING TROP2-EXPRESSING TUMOR CELLS

(71) Applicant: KISOJI BIOTECHNOLOGY INC., Montreal (CA)

(72) Inventors: Wenyang HOU, Montreal (CA); Shugang YAO, San Diego, CA (US); Luis DA CRUZ, Montreal (CA); David S. YOUNG, Montreal (CA)

(21) Appl. No.: 19/349,296

(22) Filed: Oct. 3, 2025

Related U.S. Application Data
(63) Continuation of application No. PCT/CA2024/050435, filed on Apr. 4, 2024.
(60) Provisional application No. 63/457,092, filed on Apr. 4, 2023.
(60) Provisional application No. 63/535,531, filed on Aug. 30, 2023.
(60) Provisional application No. 63/598,055, filed on Nov. 10, 2023.

(51) Int. Cl. C07K 16/30; A61K 39/00; A61P 35/00; C07K 16/28

(52) U.S. Cl. CPC C07K 16/30; A61P 35/00; C07K 16/2803; C07K 16/2818; A61K 2039/505; C07K 2317/24; C07K 2317/31; C07K 2317/569

(57) ABSTRACT

The present disclosure generally relates to binding agents, such as antibodies and antigen binding fragments thereof, that are capable of binding to trophoblast cell surface antigen-2 (TROP2). Also disclosed herein are binding agents that are capable of targeting TROP2-expressing tumor cells and their use for the treatment of cancer. Single domain antibodies that specifically binds to amino acid residues of the extracellular domain of TROP2 are provided.

### CROSS REFERENCED APPLICATIONS

This application is a Continuation Application of International Application No. PCT/CA2024/050435, filed on Apr. 4, 2024, which claims priority to U.S. Provisional Application Ser. No. 63/457,092, filed on Apr. 4, 2023; 63/535,531, filed on Aug. 30, 2023, and 63/598,055, filed on Nov. 10, 2023; the entirety of each of which is hereby incorporated by reference.

### INCORPORATION OF SEQUENCE LISTING

The instant application contains a Sequence Listing, which has been submitted electronically in XML file format and is hereby incorporated by reference in its entirety. Said XML copy, created on Sep. 22, 2025, is named KJB-010US1_SL.XML, which is 778,240 bytes in size.

### TECHNICAL FIELD

The present disclosure generally relates to binding agents, such as antibodies and antigen binding fragments thereof, that are capable of binding to trophoblast cell surface antigen-2 (TROP2). Also disclosed herein are binding agents that are capable of targeting TROP2-expressing tumor cells and their use for the treatment of cancer. Single domain antibodies that specifically binds to amino acid residues of the extracellular domain of TROP2 are provided.

### BACKGROUND

Trophoblast cell surface antigen-2 (TROP2) is a membrane-bound protein expressed at low levels in normal adult tissues. TROP2 has pleiotropic functions including inducing cell signaling and formation of cell-cell junctions by mediating interactions with extracellular and intracellular proteins. TROP2 has an extracellular epidermal growth factor (EGF)-like domain, followed by a 23 amino acids transmembrane domain and the cytoplasmic tail. The EGF-like domain contains a cysteine-rich domain, a thyroglobulin type-1 domain, and a cysteine-poor domain. In situations of TROP2 deficiency, its role in normal tissues appears to be compensated by expression of EpCAM, which is highly related to TROP2. TROP2 is highly expressed in a wide range of late-stage epithelial cancers including breast cancer and pancreatic cancer (Stepan et al., J. Histochem and Cytochem, 2011, the entire content of which is incorporated herein by reference). Overexpression of TROP2 was shown to confer oncogenic behavior in several in vitro and in vivo models. TROP2 upregulation in solid cancers is associated with increased tumor aggressiveness, metastasis, and an overall decreased survival in large groups of difficult-to-treat cancers, making it an attractive target for cancer therapy.

TROP2 positive cancers have been successfully targeted clinically by antibody-drug conjugates (ADC) but not by naked functional antibodies. ADC's have associated drug toxicity in healthy tissues, off-target effects, and payload delivery issues. Given ADC dose-limiting side effects, narrow therapeutic windows, and efficacy limitations, there is a pressing need to improve anti-TROP2 therapeutics for patients who are poorly served by current therapies, if at all.

Camelids and cartilaginous fishes naturally produce antibodies composed of functional homodimeric heavy chain only antibodies (HCAbs) (Hamers-Casterman et al., 1993; Muyldermans and Smider, 2016). The heavy chains of HCAbs lack the first constant domain (CH1) and differs from classical antibodies by only a few amino acids substitutions normally involved in light chain pairing (Muyldermans et al., 1994; Vu et al., 1997). These substitutions (Val37Phe/Tyr, Gly44Glu, Leu45Arg, and Trp47Gly) are present in framework region 2 (FR2).

The antigen-binding fragment of HCAbs is referred to as, VHH or Nanobody®. VHHs have a molecular weight of around 15 kDa which makes them amenable to applications that require enhanced tissue penetration or rapid clearance, such as radioisotope-based imaging. However, for therapeutic applications, the VHH half-life usually needs to be increased so as to minimize renal clearance and optimize therapeutic efficacy (De Vlieger et al., Antibodies 8(1), 1-22, 2019). Although methods to increase VHH half-life such as PEGylation, N-glycosylation, HSA or other carrier protein fusions have been exploited, such constructs can introduce immunogenicity or have limited success.

Variable regions of single domain antibodies have been exploited as building blocks to make bispecific and multispecific antibodies. In some studies, bivalent constructs have been shown to be have increased avidity or affinity compared to the monovalent form (Conrath et al., 2001; Coppieters et al., 2006; Hmila et al., 2008; Simmons et al., 2006 and Hultberg et al., 2011, Jahnichen et al. (2010), Fridy et al., 2014).

Novel TROP2 binding agents including antibodies and antigen binding fragments thereof such as single domain antibodies and antigen binding fragments thereof are disclosed herein.

### SUMMARY

The present disclosure therefore generally relates to a binding agent comprising one or more antigen binding domains, wherein at least one of the one or more antigen binding domains specifically bind to trophoblast cell surface antigen-2 (TROP2).

The binding agent, or at least one of its one or more binding domains, binds to human TROP2. Accordingly, the binding agent of the present disclosure may be used for treatment of human diseases or disorders associated with expression or overexpression of TROP2.

In some embodiments, the binding agent of the present disclosure may be capable of binding to the extracellular domain (ECD) of TROP2 (as represented by amino acids 27 to 274 of TROP2 (e.g., SEQ ID NO:39 or SEQ ID NO:40), see Goldenberg et al., 2018, Ikeda, M. et al., 2015, the entire contents of which are incorporated herein by reference).

In some embodiments, the one or more antigen binding domains of the binding agent comprise a complementarity determining region (CDR), a heavy chain variable region or a heavy chain of a single domain antibody (sdAbs) disclosed herein.

The single domain antibodies of the present disclosure have unique binding features and activity. For example, the complementarity determining region 3 (CDR3) of the sdAbs forms a loop fitting into a groove within the cysteine rich domain (CRD) of the extracellular domain (ECD) of TROP2. Amino acid residues of the CDR3 and of the framework region 2 (FR2) interact with amino acid residues of TROP2 cysteine rich domain and together form a clip around the CRD as a bi-paratopic antibody. These unique properties confer high affinity and a slow dissociation rate to the sdAbs disclosed herein. Surprisingly, amino acid residues of the complementarity determining region 1 (CDR1) and of the complementarity determining region 2 (CDR2) do not appear to interact with amino acid residues of the CRD. Variation in the amino acid sequence of CDR1 and CDR2 is therefore contemplated as such variation may not affect binding to TROP2. The one or more antigen binding domains therefore comprise, at least CDR3 amino acid residues.

In some instances, the one or more antigen binding domains may comprise CDR3 amino acid residues and framework region 2 (FR2) amino acid residues.

In other instances, the one or more antigen binding domains may comprise CDR3 amino acid residues and framework region 2 (FR2) amino acid residues and optionally CDR1 and/or CDR2 amino acid residues.

In a further embodiment, the one or more antigen binding domains may comprise CDR1, CDR2 and CDR3 amino acid residues.

In yet a further embodiment, the one or more antigen binding domains may comprise CDR1, FR2, CDR2 and CDR3 amino acid residues.

In some embodiments, the CDR1, CDR2, CDR3 and FR2 amino acid residues correspond to those of a single domain antibody disclosed herein.

In accordance with the present disclosure, the CDRs may be identified using the Kabat numbering scheme (e.g., Kabat, J Immunol., 147:1709-19 (1991); Chothia C, Lesk A M, J Mol Biol. August 20; 196(4):901-17 (1987)).

Alternatively, the CDRs may be identified using the IMGT numbering scheme (e.g., Lefranc, M.-P., The Immunologist, 7, 132-136 (1999)).

In some instances, the CDR1, CDR2, CDR3 and/or FR2 correspond to the Kabat CDR1, CDR2, CDR3 and/or FR2.

In other instances, the CDR1, CDR2, CDR3 and/or FR2 correspond to the IMGT CDR1, CDR2, CDR3 and/or FR2.

In some embodiment, the one or more antigen binding domains may comprise amino acid residues of variable region.

For example, in some embodiments, the one or more antigen binding domains may comprise an amino acid sequence of a camelid VH or VHH. In other embodiments, the one or more antigen binding domains may comprise an amino acid sequence of a mouse VH. In yet other embodiments, the one or more antigen binding domains may comprise an amino acid sequence of human VH. In a further embodiment, the one or more antigen binding domains may comprise an amino acid sequence of humanized VH or VHH (e.g., a humanized camelid VH or VHH, or a humanized mouse VH).

In some instances, the present disclosure relates to anti-TROP2 single domain antibodies that are capable of inducing antibody-dependent cellular cytotoxicity (ADCC) and/or antibody-dependent cellular phagocytosis (ADCP). Moreover, in other instances, the present disclosure relates to anti-TROP2 single domain antibodies that are capable of internalization making them amenable to deliver drug payloads into the tumor microenvironment or into tumor cells. In yet other instances, the present disclosure relates to anti-TROP2 single domain antibodies that interfere/block TROP2 function.

Although, binding agents of the present disclosure may have activity against tumor cells when unconjugated, the present disclosure also encompasses conjugation of binding agents with therapeutic moiety. Conjugation may be particularly beneficial when binding agents have no or low activity against tumor cells.

Accordingly, the present disclosure relates to an unconjugated (naked) single domain antibody that is capable or binding TROP2. The present disclosure also relates to an antibody drug conjugate (ADC) comprising a single domain antibody that is capable or binding TROP2 and a drug payload.

The binding function of sdAbs is conferred by a single polypeptide chain namely, the heavy chain variable region. This unique property provides formatting flexibility. For example, multiple sdAbs or sdAb antigen binding domains may be fused into a single polypeptide chain conferring multivalence and/or multispecificity. In addition, multiple polypeptide chains may be assembled to increase the diversity (multi-paratopic binding) or avidity of interactions with TROP2. Moreover, the sdAbs antigen binding domains may be fused to any type of polypeptide chain including for example and without limitation, fusion with a heavy chain and/or light chain of a native antibody or antigen binding fragment thereof, with protein scaffold, with immune cell modulating agent and the like.

As such, the present disclosure not only relates to the single domain antibodies disclosed herein but also more generally to binding agents that comprise one or more antigen binding domains of the single domain antibodies disclosed herein.

In addition, the present disclosure relates to binding agents that compete with the single domain antibodies of the present disclosure. For example, single domain antibodies that compete with one of more single domain antibodies disclosed herein for the binding of at least one epitope comprising amino acid residues of the extracellular domain (ECD) of TROP2 are provided.

The binding agent, or antigen binding domain(s) of the binding agent may come from various sources including from animals capable of producing single domain antibodies such as camelids, shark, transgenic animals, from screening of an antibody library etc. For example, antibodies may be obtained from hybridoma after immunization of rodents (mouse, rats etc.) or from larger mammals such as camelids. In some embodiments, the animal may be immunized with a TROP2 antigen.

In accordance with the present disclosure, the binding agent may comprise two heavy chain variable regions. Each heavy chain variable regions may be on same or separate polypeptide chains. In some instances the two heavy chain variable region are linked to dimerization domain allowing the formation of a dimer.

In some embodiments, the binding agent comprises a polypeptide chain comprising in a N- to C-terminal fashion, a) an antigen binding domain of a single domain antibody, b) optionally a linker and c) a dimerization domain.

In some embodiments, the binding agent comprises a polypeptide chain comprising in a N- to C-terminal fashion, a) a dimerization domain, b) optionally a linker and c) an antigen binding domain of a single domain antibody.

In other embodiments, the binding agent comprises a polypeptide chain comprising in a N- to C-terminal fashion, a) an antigen binding domain of a single domain antibody, b) a linker, c) a dimerization domain, and one or more of d) a linker and e) an antigen binding domain of a single domain antibody.

In some embodiments, the antigen binding domain of a) and the antigen binding domain of e) are different. In some embodiments, the antigen binding domain of a) and the antigen binding domain of e) are the same. In some embodiments, the linker of b) and the linker of d) are the same. In some embodiments, the linker of b) and the linker of d) are different.

In yet other embodiments, the binding agent may have the format set forth in formula I, formula II, formula III, formula IIIa and formula IIIb, formula IV, formula V, formula VI, formula VII or formula VIII disclosed herein.

In accordance with the present disclosure, the binding agent may comprise additional amino acid sequence or moieties (small molecules, labels and the like) at an N- and/or C-terminus. For example, in accordance with the present disclosure, the binding agent may comprise additional antigen binding domain(s) of single domain antibody(ies) at an N- or C-terminus. The additional antigen binding domain(s) may be separated from the core by one or more linkers.

In accordance with the present disclosure, the binding agent or antigen binding domain may comprise a sequence as set forth herein. In accordance with the present disclosure, the single domain may comprise a sequence as set forth herein.

In some embodiments, the binding agent or at least one of the one or more antigen binding domains comprise the CDR1, CDR2, CDR3, FR2 or combination thereof of the heavy chain variable domain set forth in any one of SEQ ID NOs: SEQ ID NO:231, SEQ ID NO:232, SEQ ID NO:239, SEQ ID NO:240, SEQ ID NO:247, SEQ ID NO:295, SEQ ID NO:296, SEQ ID NO:297, SEQ ID NO:311, SEQ ID NO:318, SEQ ID NO:325, SEQ ID NO:332, SEQ ID NO:339, SEQ ID NO:346, SEQ ID NO:353, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:491, SEQ ID NO:493, SEQ ID NO:496, SEQ ID NO:502, or SEQ ID NO:503.

In some embodiments, the binding agent or at least one of the one or more antigen binding domains are capable of binding to human trophoblast cell surface antigen-2 (human TROP2) and comprise an amino acid sequence at least 65% identical, at least 70% identical, at least 75% identical, at least 80% identical, at least 85% identical, at least 90% identical, at least 95% identical, at least 99% identical or identical to the amino acid sequence set forth in any one of SEQ ID NOs: SEQ ID NO:231, SEQ ID NO:232, SEQ ID NO:239, SEQ ID NO:240, SEQ ID NO:247, SEQ ID NO:295, SEQ ID NO:296, SEQ ID NO:297, SEQ ID NO:311, SEQ ID NO:318, SEQ ID NO:325, SEQ ID NO:332, SEQ ID NO:339, SEQ ID NO:346, SEQ ID NO:353, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:491, SEQ ID NO:493, SEQ ID NO:496, SEQ ID NO:502, or SEQ ID NO:503.

In some embodiments, the binding agent or at least one of the one or more antigen binding domains comprise a variable region having a sequence set forth herein (including original or humanized sequences) and comprising from one to five amino acid substitutions, from one to five amino acid deletions or from one to five amino acid additions thereof.

Particular embodiments of the present disclosure includes a binding agent such as for example, a single domain antibody that comprises one or more antigen binding domains, wherein at least one of the one or more antigen binding domains binds to trophoblast cell surface antigen-2 (TROP2) and comprises:
a) a heavy chain complementarity determining region 1 (CDRH1) having the amino acid sequence set forth in SEQ ID NO:225, a heavy chain complementarity determining region 2 (CDRH2) having the amino acid sequence set forth in SEQ ID NO:226, SEQ ID NO:234, SEQ ID NO:242, SEQ ID NO:320, SEQ ID NO: 392 and a heavy chain complementarity determining region 3 (CDRH3) having the amino acid sequence set forth in SEQ ID NO:227 or;
b) a heavy chain complementarity determining region 1 (CDRH1) having the amino acid sequence set forth in SEQ ID NO:228, a heavy chain complementarity determining region 2 (CDRH2) having the amino acid sequence set forth in SEQ ID NO:229, SEQ ID NO:237, SEQ ID NO:245 or SEQ ID NO:323 or SEQ ID NO:395 and a heavy chain complementarity determining region 3 (CDRH3) having the amino acid sequence set forth in SEQ ID NO:230.

In more specific embodiments of the present disclosure the at least one of the one or more antigen binding domains of the binding agent or single domain antibody comprises:
a) a heavy chain complementarity determining region 1 (CDRH1) having the amino acid sequence set forth in SEQ ID NO:225, a heavy chain complementarity determining region 2 (CDRH2) having the amino acid sequence set forth in SEQ ID NO:226, SEQ ID NO:234, SEQ ID NO:242, SEQ ID NO:320, SEQ ID NO: 392 and a heavy chain complementarity determining region 3 (CDRH3) having the amino acid sequence set forth in SEQ ID NO:227 or;
b) a heavy chain complementarity determining region 1 (CDRH1) having the amino acid sequence set forth in SEQ ID NO:228, a heavy chain complementarity determining region 2 (CDRH2) having the amino acid sequence set forth in SEQ ID NO:229, SEQ ID NO:237, SEQ ID NO:245 or SEQ ID NO:323 or SEQ ID NO:395 and a heavy chain complementarity determining region 3 (CDRH3) having the amino acid sequence set forth in SEQ ID NO:230 and;
   an amino acid sequence at least 80% identical, at least 85% identical, at least 90% identical, at least 95% identical, at least 99% identical or identical to the amino acid sequence set forth in SEQ ID NO:373, SEQ ID NO:232, SEQ ID NO:231, SEQ ID NO:295, SEQ ID NO:296, SEQ ID NO:297, SEQ ID NO:372, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:239, SEQ ID NO:240, SEQ ID NO:383, SEQ ID NO:384, SEQ ID NO:385, SEQ ID NO:386, SEQ ID NO:387, SEQ ID NO:247, SEQ ID NO:388, SEQ ID NO:325, SEQ ID NO:390, SEQ ID NO:397, or SEQ ID NO:398.

In other specific embodiments, the at least one of the one or more antigen binding domains of the binding agent or single domain antibody comprises a heavy chain complementarity determining region 1 (CDRH1) having the amino acid sequence set forth in SEQ ID NO:488, a heavy chain complementarity determining region 2 (CDRH2) having the amino acid sequence set forth in SEQ ID NO:489 and a heavy chain complementarity determining region 3 (CDRH3) having the amino acid sequence set forth in SEQ ID NO:490 and an amino acid sequence at least 80% identical to the amino acid sequence set forth in any one of SEQ ID NO: 493, SEQ ID NO:496 or SEQ ID NO:491.

In yet other particular embodiments, the at least one of the one or more antigen binding domains of the binding agent or single domain antibody comprises a heavy chain complementarity determining region 1 (CDRH1) having the amino acid sequence set forth in SEQ ID NO:500, a heavy chain complementarity determining region 2 (CDRH2) having the amino acid sequence set forth in SEQ ID NO:489 and a heavy chain complementarity determining region 3 (CDRH3) having the amino acid sequence set forth in SEQ ID NO:501 and an amino acid sequence at least 80% identical to the amino acid sequence set forth in any one of SEQ ID NO:503 or SEQ ID NO:502.

In a further particular embodiment, the at least one of the one or more antigen binding domains of the binding agent or single domain antibody comprises a heavy chain complementarity determining region 1 (CDRH1) having the amino acid sequence set forth in SEQ ID NO:308, a heavy chain complementarity determining region 2 (CDRH2) having the amino acid sequence set forth in SEQ ID NO:309 and a heavy chain complementarity determining region 3 (CDRH3) having the amino acid sequence set forth in SEQ ID NO:310 and an amino acid sequence at least 80% identical to the amino acid sequence set forth in any one of SEQ ID NO:311 or SEQ ID NO:389.

In some instances, the at least one of the one or more antigen binding domains comprises an amino acid sequence as set forth in SEQ ID NO:539.

In exemplary embodiments, the binding agent of any of the preceding embodiments, wherein the at least one of the one or more antigen binding domains comprises an amino acid sequence as set forth in SEQ ID NO:540.

In other exemplary embodiments, the binding agent of any of the preceding embodiments, wherein the at least one of the one or more antigen binding domains comprises an amino acid sequence as set forth in SEQ ID NO:541.

In exemplary embodiments, the binding agent of any of the preceding embodiments, wherein the at least one of the one or more antigen binding domains comprises an amino acid sequence as set forth in any one of SEQ ID NOs:514-516.

In other exemplary embodiments, the binding agent of any of the preceding embodiments, wherein the at least one of the one or more antigen binding domains comprises an amino acid sequence as set forth in any one of SEQ ID NOs:517-519.

In other exemplary embodiments, the binding agent of any of the preceding embodiments, wherein the at least one of the one or more antigen binding domains comprises an amino acid sequence as set forth in any one of SEQ ID NOs:520-522.

In exemplary embodiments, the binding agent of any of the preceding embodiments, wherein the at least one of the one or more antigen binding domains comprises an amino acid sequence as set forth in any one of SEQ ID NOs:523-525.

In other exemplary embodiments, the binding agent of any of the preceding embodiments, wherein the at least one of the one or more antigen binding domains comprises an amino acid sequence as set forth in any one of SEQ ID NOs:526-528.

In an exemplary embodiment, the binding agent or antigen binding domain(s) of the present disclosure may have ADCP activity towards cells expressing human TROP2 and Cluster of Differentiation 47 (CD47) (Uniprot Accession No. Q08722-1). An exemplary and non-limiting embodiment of a CD47 antigen is provided in SEQ ID NO:176.

Binding agents comprising one or more antigen binding domains capable of binding to TROP2 and one or more antigen binding domain capable of binding to CD47 and/or to PD-1 are therefore encompassed by the present disclosure.

In some embodiments, the binding agent is an antibody or an antigen binding fragment thereof (e.g., single domain antibody or antigen binding fragment thereof), an antibody-like molecule, a protein scaffold, an immune cell modulating agent or the like.

The present disclosure also relates to a binding agent that is capable of competing with one or more binding agents disclosed herein.

Other aspects and embodiments of the present disclosure relate to a composition or pharmaceutical composition comprising the binding agent disclosed herein and a pharmaceutically acceptable carrier.

Yet other aspects and embodiments of the present disclosure relate to a pharmaceutical composition comprising a conjugated binding agent as disclosed herein and a pharmaceutically acceptable carrier.

Yet other aspects and embodiments of the present disclosure relate to a nucleic acid or set of nucleic acids encoding the binding agent disclosed herein.

Additional aspects and embodiments of the present disclosure relate to a vector comprising a nucleic disclosed herein or a set of vectors each comprising a nucleic acid disclosed herein Further aspects and embodiments of the present disclosure relate to a cell expressing the binding agent disclosed herein.

In some embodiments, the cells are engineered to express a binding agent such as for example, an antibody or an antigen binding fragment thereof or an antibody-like molecule.

Accordingly, the present disclosure encompasses isolated cells that express an antibody or an antigen binding fragment thereof or an antibody-like molecule.

In other embodiments, the cells are engineered to express a binding agent such as for example, a chimeric antigen receptor (CAR) construct. Accordingly, the present disclosure encompasses T-cells, NK cells, monocytes or macrophages that express a chimeric antigen receptor (CAR) construct.

Additional aspects and embodiments of the present disclosure relate to a cell comprising the nucleic acid or the vector disclosed herein.

Further aspects and embodiments of the present disclosure relate to a kit comprising the binding agent disclosed herein.

In some embodiments, the kit may comprise a binding agent that binds to TROP2 and a binding agent that binds to a macrophage marker. For example, the kit may comprise an antibody or an antigen binding fragment thereof that specifically binds to TROP2. In some instances, the kit may comprise a binding agent as described herein. The kit may also comprise an antibody that binds to a macrophage marker, such as for example and without limitations an antibody or antigen binding fragment thereof that binds to CD68 (human CD68).

Yet further aspects and embodiments of the present disclosure relate to a kit comprising the nucleic acid or vector disclosed herein.

Yet further aspects and embodiments of the present disclosure relate to a kit comprising the cells disclosed herein.

In an additional aspect and embodiments, the present disclosure relates to a method of treating a disorder or disease comprising administering the binding agent, composition or pharmaceutical composition disclosed herein.

In some embodiments, the TROP2 binding agent, composition or pharmaceutical composition disclosed herein is administered to a subject having cancer selected from epithetical cancer, lung cancer (such as small cell lung cancer, non small cell lung cancer), myeloma, prostate cancer, breast cancer (such as triple negative breast cancer), rectal cancer, pancreatic cancer, glioblastoma, cervical cancer, colorectal cancer, gastric cancer, ovarian cancer, thyroid cancer, stomach cancer, cancer of the urinary bladder, cancer of the uterus, esophageal cancer, head and neck cancer, blood cancer.

In other embodiments, the TROP2 binding agent, composition or pharmaceutical compositions disclosed herein is administered to a subject having a metastatic cancer.

Other aspects of the disclosure relate to a method of treating cancer, which comprises administering the binding agent disclosed herein to a subject having a tumor expressing TROP2 and expressing one or more macrophage markers such as, for example and without limitations, CD68.

In other aspects and embodiments, the present disclosure relates to a method of making the binding agent disclosed herein by transforming cells with one or more vectors comprising the nucleic acid disclosed herein and optionally conjugating the binding agent with a therapeutic moiety, detectable moiety, or a protein allowing an extended half-life or to nanoparticles.

Further scope, applicability and advantages of the present disclosure will become apparent from the non-restrictive detailed description given hereinafter. It should be understood, however, that this detailed description, while indicating exemplary embodiments of the disclosure, is given by way of example only, with reference to the accompanying drawings.

### BRIEF DESCRIPTION OF THE DRAWINGS

FIGS. 1A-1D: graphs illustrating the tumor volume over time of SCID mice bearing MDA-MB-453 (FIG. 1A), MDA-MB-231 (FIG. 1B), BxPC-3 (FIG. 1C) or T.Tn (FIG. 1D) treated with the KD001, KD002, KD003, KD004 or KD005 anti-TROP2 sdAbs or with PBS.

FIG. 1E: histogram showing binding of KD001, KD002, KD003, KD004 or KD005 anti-TROP2 sdAbs to MDA-MB-453, MDA-MB-231, BxPC3, and A375 cells compared with AR47A6.4.2 anti-TROP2 monoclonal antibody as analyzed by flow cytometry. Negative control is sdAb that binds HEWL (NC).

FIG. 2A: graph of in vivo dose/response study of weekly treatment of KD001, KD002, KD003, KD004 or KD005 anti-TROP2 sdAbs at doses of 0.5 mg/kg, 2 mg/kg, 8 mg/kg and 30 mg/kg i.p. in MDA-MB-453 implanted NCG mice tumor model. Negative control is PBS.

FIG. 2B: graph of in vivo weekly treatment of large tumor with KD004 at 8 mg/kg, once weekly (QW) i.p. in MDA-MB-453 implanted NCG mice tumor model. Negative control is PBS.

Treatment was discontinued at around day 70, and replaced with PBS, and the negative control treated mice were treated with KD004 at 8 mg/kg QW.

FIG. 2C: graph showing the anti-tumor effect of various treatment regimen with KD004 at a dose of 8 mg/kg with the indicated frequencies i.p. in MDA-MB-453 implanted NCG mice tumor model. Negative control is PBS.

FIG. 2D: graph showing the anti-tumor effect of various treatment regimen with KD002 or KD005 at a dose of 0.1 mg/kg i.p. in MDA-MB-453 implanted NCG mice tumor model. Negative control is PBS.

FIG. 2E: Anti-TROP2 in vivo pharmacokinetic parameters.

FIG. 3: graph showing tumor volume over time of MDA-MB-453 implanted NCG mice treated with KD004 at 8 mg/kg i.p. at the indicated intervals in the presence or absence of human PBMCs. Negative control is PBS.

FIG. 4A: histogram showing cell viability of MDA-MB-453 cells following 12 days treatment with 1 μM KD001, KD002, KD003, KD004 or KD005 anti-TROP2 sdAbs in comparison with AR47A6.4.2 anti-TROP2 monoclonal antibody. Negative controls include sdAb that binds HEWL (NC) or untreated cells.

FIG. 4B image of MDA-MB-453 cells at 4× resolution after 12 days of treatment with KD001, KD002, KD003, KD004 or KD005 anti-TROP2 sdAb. Negative controls include sdAb that binds HEWL (NC) or untreated cells.

FIG. 5A: histogram showing cell viability of MDA-MB-453 cells reflecting the ADCC activity of KD001, KD002, KD003, KD004 or KD005 anti-TROP2 sdAbs. Negative controls include untreated cells (No Ab), no effector cells, no target cells or sdAb that binds HEWL (NC). Comparisons includes cells treated with human IgG1 anti-TROP2 antibodies generated with the sequences from clones hTINA-1 and h7E6.

FIG. 5B: sdAbs tumor growth inhibition (TGI) was tested in vivo in NCG and SCID mice having different effector function background and implanted with either MDA-MB-231 or MDA-MB-453 cells s.c. Administration of KD002 or KD004 was initiated when average tumor volume reached 100 mm³.

FIGS. 6A-6B: histograms illustrating internalization of KD001, KD002, KD003, KD004 or KD005 anti-TROP2 sdAbs (FIG. 6A) or AR47A6.4.2 (FIG. 6B) in MDA-MB-453 cells after 4 or 24 hours. Negative controls include sdAb that binds HEWL (NC) or medium only.

FIG. 6C: graph illustrating internalization of KD001, KD002, KD003, KD004 or KD005 anti-TROP2 sdAbs in MDA-MB-453. Internalization was assessed by conjugation of HCAbs with a pH-sensitive probe and monitored by flow cytometry (Em. 560 nm).

FIG. 6D: Confocal microscopy images of MDA-MB-453 cells showing KD002 and KD005 antibody binding and internalization were taken at 0, 6 and 24 hours. For each time point, one representative cell is shown. The cells were stained with anti-human IgG Fc-FITC and the nuclei were counterstained with DAPI. Images were obtained using confocal fluorescence microscopy. Scale bar, 10 μM.

FIGS. 7A-7D: binding of KD001, KD002, KD003, KD004, KD005, KD006 or KD007 anti-TROP2 sdAbs to CHO (FIG. 7A) or HEK parental cells (FIG. 7C) or CHO (FIG. 7B) or HEK cells expressing human TROP2 (FIG. 7D) compared with negative control (sdAb that binds HEWL (NC)) or comparator monoclonal antibodies AR47A6.4.2, hTINA-1, h7E6 and hRS7 as assessed by flow cytometry.

FIG. 7E: Bar graph illustrating binding of KD001, KD002, KD003, KD004, KD005, KD006 or KD007 anti-TROP2 sdAbs to recombinant human EpCAM (rhEpCAM) and to recombinant human IGFBP-1 (rhIGFB-1) proteins.

FIGS. 8A-8E: graph illustrating competition assays performed with either His tag conjugated KD001 (FIG. 8A), KD002 (FIG. 8B), KD003 (FIG. 8C), KD004 (FIG. 8D) or KD005 (FIG. 8E) and each of KD001, KD002, KD003, KD004 or KD005 anti-TROP2 sdAbs for binding to human recombinant TROP2.

FIG. 9A: graph illustrating competition assays between His tag conjugated KD004 and hRS7 or AR47A6.4.2 antibodies for the binding of human recombinant TROP2.

FIG. 9B: graph illustrating competition assays between His tagged KD006 and KD007 anti-TROP2 sdAbs for the binding of human recombinant TROP2.

FIG. 10A: schematic showing the various constructs used to map the binding domain of KD001, KD002, KD003, KD004 or KD005 anti-TROP2 sdAbs.

FIG. 10B: picture of immunoblots performed with KD001, KD002, KD003, KD004 or KD005 anti-TROP2 sdAbs or with AR47A6.4.2 and hRS7 on human TROP2 extracellular domain (ECD) and human TROP2 cysteine poor domain (CPD). Negative control includes sdAb that binds HEWL (NC).

FIGS. 11A-11E: pictures of immunoblots performed with KD001, KD002, KD003, KD004 or KD005 anti-TROP2 sdAbs on different forms of recombinant human TROP2 (non-denaturated, linear, reduced, deglycosylated using PNGase F).

FIGS. 12A-12D: graph showing binding of KD001, KD002, KD003, KD004 or KD005 anti-TROP2 sdAbs on recombinant cynomolgus/Rhesus TROP2 protein (FIG. 12A), on recombinant human TROP2 protein (FIG. 12B), on mouse TROP2 protein (FIG. 12C) or on rat TROP2 protein (FIG. 12D). Negative control includes sdAb that binds HEWL. Positive control includes anti-TROP2 polyclonal antibody.

FIG. 12E: table illustrating affinity of KD001, KD002, KD003, KD004 or KD005 anti-TROP2 sdAbs to human TROP2 and to cynomolgus TROP2 by SPR.

FIG. 12F: table illustrating affinity of KD001, KD002, KD003, KD004 or KD005 anti-TROP2 sdAbs to human TROP2 with ka, kd and KD indicated.

FIGS. 13A-13B: histogram showing the binding of the indicated TROP2 antibodies at 100 nM to HEK cells transfected with TROP2 constructs with various cysteine-rich domain truncations as indicated.

FIG. 13C: graph showing the tumor growth of in the xenograft model of T.Tn in the SCID mice upon treatment with anti-TROP2 antibodies at 8 mg/kg i.p., once per week. Treatment with KD006 led to a tumor growth inhibition of 48.6% in the xenograft model of T.Tn.

FIG. 14A: graph of the antibody-mediated cellular phagocytosis activity of IgG1- or IgG4 versions of KD002 and KD004 against MDA-MB-453 tumors.

FIG. 14B: graph showing CD47 and TROP2 expression level on MDA-MB-453, MDA-MB-231, and NCI-H292 cells measured by staining with anti-CD47 (Biolegend, cat #323124) and anti-TROP2 (Biolegend, cat #363804) antibodies at 1/100 dilutions. Data are shown as fold change of CD47 and TROP2 level relative to the isotype control staining.

FIGS. 14C-14E: graphs showing the dose-response ADCP activity of KD002 and KD004 TROP2 antibodies against the MDA-MB-453 (FIG. 14C), MDA-MB-231 (FIG. 14D), and NCI-H292 (FIG. 14E) cells. The ADCP assays were performed with the Promega ADCP assay kits (Promega, cat #CS314906). Testing antibodies were serial diluted from 1.88 nM with 1/2.2 dilution. TROP2 antibodies KD002 and KD004 showed a high level of ADCP activity against MDA-MB-453 cells, whereas relatively low ADCP activity on MDA-MB-231 and NCI-H292 cells.

FIG. 14F: graph illustrating the ADCP activity of KD002 and KD004 and the corresponding IgG4 version; KD017 and KD018 against MDA-MB-453, MDA-MB-231, and NCI-H292 cells. Assay was performed at the antibody concentration of 1 nM.

FIG. 15A: graph illustrating the CD47 expression level on MDA-MB-231 and NCI-H292 cells after CD47 knockdown with siRNA. The CD47 siRNA and control siRNA were used in the knockdown assay (Thermofisher CD47 silencer select pre-designed siRNA (Cat #4392421) and silencer negative control siRNA (Cat #AM4635).

FIGS. 15B-15C: graphs showing the dose-response ADCP activity of KD002 and KD005 TROP2 antibodies against the MDA-MB-231 (FIG. 15B), and NCI-H292 cells (FIG. 15C) upon CD47 knockdown. Testing antibodies were serial diluted from 2.1 nM with 1/2.5 dilution.

FIG. 15D: graphs illustrating the ADCP activity of KD002 and KD005 and the corresponding IgG4 version, KD017 and KD019 against the MDA-MB-453, MDA-MB-231, and NCI-H292 cells upon inhibition of CD47 expression with siRNA (siCD47) or not (siNC). Assay was performed at the antibody concentration of 1 nM.

FIG. 15E: graphs showing in vivo efficacy of TROP2-CD47 bispecific antibodies in the xenograft model of MDA-MB-231 in SCID mice. Mice were treated with KD004 at 8 mg/kg, once per week and KD065 and KD066 were administered at the same molarity (11 mg/kg) i.p.

FIG. 16A: plots showing the THP-1 differentiated M1 and M2 macrophages upon treatment with PMA and cytokines. THP-1 cells showed upregulation of CD68 after PMA treatment. The M1 cells showed upregulation of CD38 and CD86 after treatment with IFN-7 and LPS. M2 macrophages showed upregulation of CD206 and downregulation of CD38 after treatment with IL-4 and IL-13.

FIG. 16B: bar graph showing the percentage of phagocytosed M1 or M2 macrophages upon treatment with KD002, KD004 or KD005 at 10 nM as illustrated by gating on the pHrodo positive and Celltrace Violet positive cells.

FIG. 17A: bar graph illustrating the binding of TROP2 antibodies at 25 nM to TROP2 recombinant proteins by ELISA using an anti-IgG4 Fc secondary antibody.

FIG. 17B: graph showing the anti-tumor effect of KD002 and KD017 at a dose of 8 mg/kg i.v. in MDA-MB-453 implanted NCG mice tumor model. Negative control is PBS.

FIG. 18A: graph showing the dose-response ADCP activity of KD002 and KD005 TROP2 antibodies against the MDA-MB-453 (upper panel), MDA-MB-468 (middle panel), and T.Tn cells (lower panel). The ADCP assays were performed using the Promega ADCP assay kits (Promega, cat #CS314906).

FIG. 18B: graph showing the anti-tumor effect of KD002 and KD005 in the MDA-MB-453 (upper panel), MDA-MB-468 (middle panel), and T.T.n (lower panel) xenograft tumor models. Testing antibodies were administered with 8 mg/kg i.p., once per week.

FIG. 19A: Photomicrographs of IHC staining of mouse macrophages with F4/80 antibody on the xenograft tumors collected from COLO205, CAPAN1, MDA-MB-231, T.T.n and MDA-MB-453 xenograft models treated with the TROP2 antibodies KD002 and KD005 at the dose level of 8 mg/kg. Tumors were collected at the end of the study.

FIG. 19B: Human colon adenocarcinoma tumor tissue microarray with KD067, KD068 or isotype control KD069 antibody. The human multiple tumor tissue microarray was purchased from Novus Biologics (cat #NBP2-42056) and stained with the antibodies at 2 μg/ml.

FIG. 19C: Human breast invasive ductal carcinoma tumor tissue microarray with KD067, KD068 or isotype control KD069 antibody. The human multiple tumor tissue microarray was purchased from Novus Biologics (cat #NBP2-42052) and stained with the antibodies at 2 μg/ml.

FIG. 19D: Human head and neck tumor tissue microarray with KD067, KD068 or isotype control KD069 antibody (H&N squamous cell carcinoma). The human multiple tumor tissue microarray was purchased from Novus Biologics (cat #NBP2-42056) and stained with the antibodies at 2 μg/ml.

FIG. 19E: Human kidney transitional cell carcinoma microarray with KD067, KD068 or isotype control KD069 antibody. The human multiple tumor tissue microarray was purchased from Novus Biologics (cat #NBP2-42052) and stained with the antibodies at 2 μg/ml.

FIG. 20A: X-ray crystal structure of the extracellular domain of TROP2 protein with translucent surface rendering. The relative position of the Cystein-Rich Domain (CRD), Thyroglobulin Domain (TY-1), and Cystein-Poor Domain (CPD) are indicated.

FIG. 20B: X-ray cocrystal of KD012 with human TROP2 (surface rendering shown). The contact region on TROP2 is shown on the surface of TROP2 in dark grey. The contacts between KD012 and TROP2 occur mainly in the CRD of TROP2 and CDR3 and framework 2 of KD012. CDRs and framework 2 are indicated with arrows.

FIG. 20C: X-ray cocrystal structure of KD012 with human TROP2. KD102 is depicted as a ribbon diagram. Selected residues on KD012 are shown, with the interacting residues on TROP2 shown in dark grey.

FIG. 20D: shows amino acid residues involved in the interaction between KD012 and human TROP2 (within 4.5 Å; CDRs annotated in accordance with IMGT numbering).

FIGS. 21A-21B: represents dose-response ELISA binding of KD002 and humanized variants on human TROP2 recombinant protein. TROP2 antibodies were serially diluted from 200 nM to 0.00256 nM with 1/5 dilutions.

FIGS. 22A-22B: represents dose-response ELISA binding of KD005 and humanized variants on human TROP2 recombinant protein. TROP2 antibodies were serially diluted from 200 nM to 0.00256 nM with 1/5 dilutions.

FIGS. 23A-23B: bar graphs illustrating the relative light units of KD002 humanized variants compared to KD002 at 0.5 nM (FIG. 23A) or 0.1 nM (FIG. 23B) as measured with the Promega ADCP reporter assays.

FIGS. 24A-24B: bar graphs showing the relative light units of KD005 and KD005 humanized variants at 0.5 nM (FIG. 24A) and 0.1 nM (FIG. 24B) as measured with the Promega ADCP reporter assays.

FIGS. 24C-24I: dose-response ADCP assays carried out in MDA-MB-453 cells with KD045 (FIG. 24C), KD047 (FIG. 24D), KD048 (FIG. 24E), KD049 (FIG. 24F), KD053 (FIG. 24G), KD059 (FIG. 24H) and KD063 (FIG. 24I).

FIGS. 25A-25B: bar graphs showing the relative light units of KD002 humanized variants compared to KD002 at 0.5 nM (FIG. 25A) or 0.1 nM (FIG. 25B) as measured with the Promega ADCC reporter assays.

FIGS. 26A-26B: bar graphs showing the relative light units of KD005 and KD005 humanized variants at 0.5 nM (FIG. 26A) and 0.1 nM (FIG. 26B) as measured with the Promega ADCC reporter assays.

FIG. 27: Alignment between KD001, KD002, KD003, KD004 and KD005 variable regions.

FIG. 28: Alignment and consensus sequences of KD002 variants.

FIG. 29: Alignment of KD002 variants selected for their ADCC and ADCP activity.

FIG. 30: Alignment and consensus sequences of KD005 variants.

FIG. 31: Alignment of KD005 variants selected for their binding activity.

FIG. 32: Alignment of KD005 variants selected for their ADCP activity.

FIG. 33: Alignment of KD005 variants selected for their ADCC activity.

FIG. 34: Alignment of KD005 variants selected based on all characteristics.

FIG. 35: KD005 caused complete tumor regression with long duration of response in large Colo201 colorectal cancer xenograft model in SCID mice.

FIG. 36: KD005 treatment caused tumor growth inhibition in the NCI-H2228 Non-small cell lung cancer xenograft model in SCID mice.

FIG. 37: KD005 treatment caused tumor growth inhibition in the Cal27 Head and Neck cancer xenograft model in SCID mice.

FIG. 38: KD005 treatment caused regression of the Colo201 colorectal cancer xenograft model in SCID mice. Treatment with the effector function-null LALAPG Fc mutant version of KD005 did not affect tumor growth.

FIG. 39: KD005 treatment caused regression of the MDA-MB-453 breast cancer xenograft model in NCG mice. KD005 treatment did not change tumor growth of the MDA-MB-453 xenograft model implanted in mice deficient in all Fcg Receptors (FcgR^{ko}) FIG. 40A: Table of EC50 values (nM) for KD005, KD001, KD132, KD134 binding to human TROP2 recombinant protein.

FIG. 40B: Table of EC50 values (nM) for KD001, KD132, KD134 binding to MDA-MB-453 and COLO 201 cell lines.

FIG. 40C: Table of EC50 values (nM) for KD001, KD132, KD134 ADCP activity in MDA-MB-453 and COLO 201 cells.

FIGS. 41A-41D: Treatment with KD133, KD135 and KD136 induces tumor regression in MDA-MB-453 (FIGS. 41A-41B) and COLO201 (FIGS. 41C-41D) xenograft in comparison with positive control KD005 and negative isotype control having specifity for Hen Egg-White Lysosyme (HEWL).

FIG. 42A: Table of EC50 values (nM) for KD005, KD137, KD138, KD139 binding to human TROP2 recombinant protein.

FIG. 42B: Table of EC50 values (nM) for KD005, KD138, KD139 binding to MDA-MB-453 and COLO 201 cell lines.

FIG. 42C: Table of EC50 values (nM) for KD005, KD138, KD139 ADCP activity in MDA-MB-453 and COLO 201 cells.

FIGS. 43A-43D: Treatment with KD137, KD138 and KD139 induces tumor regression in MDA-MB-453 (FIGS. 43A-43B) and COLO201 (FIGS. 43C-43D) xenograft in comparison with positive control KD005 and negative isotype control having specifity for Hen Egg-White Lysosyme (HEWL).

FIGS. 44A-44B: KD142 and KD143 binding to human TROP2 recombinant protein (FIG. 44A) and to MDA-MB-453 cells and to NCI-H292 cells (FIG. 44B).

FIGS. 45A-45B: Alignment of heavy chain variable regions of Table 15E and Table 15F.

### DETAILED DESCRIPTION

### Definitions

The use of the terms “a” and “an” and “the” and similar referents in the context of describing embodiments (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

As used herein the term “TROP2” refers to a TROP2 protein or to a polypeptide comprising the TROP2 amino acid sequence. The term “TROP2” encompasses the human TROP2 protein and any protein having at least 80% identity with the human TROP2 protein.

The term “anti-TROP2 antibody” refers to an antibody of any format that binds to the TROP2 protein such as a TROP2 antigen or to a portion thereof and includes without limitations “anti-TROP2 single domain antibody”.

The term “binding agent” refers to a molecule that comprises a polypeptidic portion (such as polypeptide chain(s)) and that is capable of specifically binding an antigen or that comprises a domain such as an antigen binding domain that is capable of specifically binding an antigen.

The term “antibody” is used in the broadest sense and encompasses various antibody formats and structures, including any immunoglobulin, monoclonal antibody, polyclonal antibody, bivalent antibody, monovalent antibody, bispecific antibody, multiple specific (multi-specific) antibody, conventional antibody, single domain antibody, single chain antibody, heavy chain only antibody, nanobody, full-length antibody, humanized antibody, chimeric antibody that binds to a specific antigen, and any antigen binding fragment that exhibits the desired antigen binding activity. An antibody can be naturally occurring (native) or the results or recombination technologies.

As used herein the expressions “single domain antibody” and “heavy chain only antibody” are used interchangeably. The term “single domain antibody” includes naturally occurring single domain antibody or heavy chain only antibody as well as single domain antibody in which the naturally occurring constant region is replaced by a dimerization domain as disclosed herein as well as variants such as humanized or chimeric single domain antibodies. When a single domain antibodies has a dimerization domain, it is generally composed of two polypeptide chains.

A “naturally occurring single domain antibody” includes antibodies produced by camelids (camelid antibodies) or by shark. “Naturally occurring single domain antibody(ies)” also encompass antibody(ies) that are produced by transgenic animals modified to express heavy chain only antibodies. Exemplary embodiments of transgenic animals are provided in international application No. PCT/CA2021/050951 filed on Jul. 21, 2021, and published on Jan. 20, 2022, under No. WO2022/011457, the entire content of which is incorporated herein by reference.

It is to be understood herein that any of the terms “KD001”, “KD002”, “KD003”, “KD004”, “KD005”, “KD006”, KD007”, “KD008”, “KD009”, “KD010”, “KD011”, “KD012”, “KD013”, “KD014”, “KD015”, “KD016”, “KD017”, “KD018”, “KD019”, “KD020”, “KD021”, “KD022”, “KD023”, “KD024”, “KD025”, “KD026”, “KD027”, “KD028”, “KD029”, “KD030”, “KD031”, “KD032”, “KD033”, “KD034”, “KD035”, “KD036”, “KD037”, “KD038”, “KD039”, “KD040”, “KD041”, “KD042”, “KD043”, “KD044”, “KD045”, “KD046”, “KD047”, “KD048”, “KD049”, “KD050”, “KD051”, “KD052”, “KD053”, “KD054”, “KD055”, “KD056”, “KD057”, “KD058”, “KD059”, “KD060”, “KD061”, “KD062”, “KD063”, “KD064”, “KD067”, “KD068”, “KD069”, “KD071”, “KD072”, “KD073”, KD074”, “KD075”, “KD076”, “KD077”, “KD078”, “KD079”, KD080”, “KD081”, “KD082”, “KD083”, “KD084”, “KD085”, KD086”, “KD087”, “KD088”, “KD089”, “KD090”, “KD091”, “KD092”, “KD093”, “KD094”, “KD095”, “KD096”, “KD097”, “KD098”, “KD099”, “KD100”, KD101”, “KD102”, “KD103”, “KD104”, “KD105”, “KD106”, “KD107”, “KD108”, “KD109”, “KD110”, “KD111”, “KD112”, “KD113”, “KD114”, “KD115”, “KD116”, “KD117”, “KD118”, “KD119”, “DK120”, “KD121”, “KD122”, “KD123”, “KD124”, “KD125”, “KD126”, “KD127”, “KD128”, KD129”, KD130”, “KD131”, “KD132”, “KD133”, “KD134”, “KD135”, “KD136”, KD137”, “KD138”, KD139”, “KD140”, or “KD141” refer to a single domain antibody that comprises two heavy chains, each having the corresponding heavy chain amino acid sequence set forth in any of Table 15A to Table 15K, Table 16, Table 18 or Table 19 or, each having the corresponding variable region amino acid sequence set forth in any of Table 15A to Table 15K, Table 16, Table 18 or Table 19.

The terms “KD065” or “KD066” refer to a binding agent that comprises two chains, each having the corresponding amino acid sequence of the polypeptide chain set forth SEQ ID NO:150 or SEQ ID NO:151 respectively or each having the corresponding variable region amino acid sequence of the polypeptide chain. In some embodiments, any one of KD065 or KD066 may have the corresponding variable region amino acid sequence of KD065 or KD66 and a different hinge and/or dimerization domain (e.g., CH2 and/or CH3) than KD065 or KD66.

The term “subject in need” as used herein refer to a subject having, suspected of having a disorder or disease associated with TROP2, TROP2 expression (e.g., expression in tissue, cells or serum) or TROP2 overexpression. The term “subject in need” also refers to a subject having, suspected of having a disorder or disease that may benefit from treatment with a binding agent targeting TROP2. A “subject in need” include a subject having cancer or suspected of having cancer.

The term “treatment” for purposes of this disclosure refers to both therapeutic treatment and prophylactic or tumor prevention measures. Subject in need of treatment include those already with the disorder as well as those prone to have the disorder or those in whom the disorder is to be prevented. Subject in need of treatment thus include subjects that already have cancer as well as those prone to have cancer or those in whom cancer is to be managed.

The term “natural” as used in the context of a sequence refers to a sequence that occurs in nature.

The term “about” or “approximately” with respect to a given value means that variation in the value is contemplated. In some embodiments, the term “about” or “approximately” shall generally mean a range within +/−10 percent, within +/−5 percent, within +/−4 percent, within +/−3 percent, within +/−2 percent or within +/−1 percent of a given value or range.

It is to be understood herein, that expressions referring to ranges of values in the format such as “from A to B”, include each individual value and any sub-range comprised and including such ranges. For example, the expression “from 1 to 10” includes sub-ranges such as and without limitations, “from 2 to 10”, “from 2 to 9”, “from 3 to 6”, “from 5 to 7” and any individual values comprised between and including 1 and 10, i.e., 1, 2, 3, 4, 5, 6, 7, 8, 9 or 10.

It is to be understood herein that the term “at least” with respect to a given value intends to include the value and superior values. For example, the term “at least 80%” includes “at least 80%”, “at least 81%”, “at least 82%”, “at least 83%”, “at least 84%”, “at least 85%”, “at least 86%”, “at least 87%”, “at least 88%”, “at least 89%”, “at least 90%”, “at least 91%”, “at least 92%”, “at least 93%”, “at least 94%”, “at least 95%”, “at least 96%”, “at least 97%”, “at least 98%”, “at least 99%”, “at least 99.1%”, “at least 99.2%”, at least 99.3%”, at least 99.4%”, at least 99.5%”, at least 99.6%”, at least 99.7%”, at least 99.8%”, at least 99.9%”, and 100%.

As used herein, the term “humanized” means that the binding agent such as an antibody or antigen binding fragment comprises CDRs or CDR amino acid residues derived from non-human animal antibodies, FR regions derived from human antibodies, and when applicable, the constant regions derived from human antibodies.

As used herein the expression “fully humanized framework region” means that the amino acid sequence of all framework regions of a given binding agent such as an antibody or antigen binding fragment is identical to that of a human antibody variable region e.g., a human germline antibody variable region sequence).

As used herein the expression “partially humanized” means that the amino acid sequence of one or more or all framework regions of the binding agent such as an antibody or antigen binding fragment is not totally identical to that of a human antibody variable region such as a germline human antibody variable region (e.g., a human germline antibody variable region sequence).

In some instances, a humanized, fully humanized or partially humanized binding agent such as an antibody or antigen binding fragment may comprises CDRs or CDR amino acid residues of a human antibody variable region (e.g., a human germline antibody variable region sequence).

A “partially humanized framework region” thus encompasses, for example and without limitations, a human framework region that contains back-mutations reintroducing an original amino acid at its original location. A “partially humanized framework region” also encompasses, for example and without limitations, a human framework region in which one or more amino acid residue has been replaced, added or deleted.

As used herein, the term “affinity” refers to the strength of non-covalent interaction between a binding agent or antigen binding domain(s) thereof and an antigen. “Affinity” is represented, for example, by K_{D} value, i.e., the ratio of dissociation rate to association rate (k_{off}/kₒₙ) when the binding between the antigen and the binding agent (e.g., antibody such as single domain antibody) reaches equilibrium. The affinity may be determined by using any conventional method known in the art, including but are not limited to, surface plasmon resonance method, microscale thermophoresis method, HPLC-MS method and flow cytometry (such as FACS) method. An antibody generally has a K_{D} value of ≤10⁻⁶ M (e.g., ≤5×10⁻⁷ M, ≤2×10⁻⁷ M, ≤10⁻⁷M, 5×10⁻⁸M, ≤2×10⁻⁸M, 10⁻⁸M, 5×10⁻⁹M, 4×10⁻⁹M, 3×10⁻⁹M, 2×10⁻⁹ M, or 10⁻⁹ M and any value 10⁻⁶). Preferably, an antibody has a K_{D} value in the nanomolar range or lower (e.g., ≤9×10⁻⁹ M, ≤8×10⁻⁹ M, ≤7×10⁻⁹ M, 6×10⁻⁹ M, ≤5×10⁻⁹ M, ≤4×10⁹ M, ≤3×10⁻⁹ M, ≤2×10⁻⁹ M, ≤1×10⁻⁹ M or lower e.g., ≤1×10⁻¹⁰ M, ≤1×10⁻¹¹ M, 1×10¹² M). Even more preferably, an antibody has a K_{D} value in the picomolar range or lower (e.g., ≤9×10⁻¹²M, ≤8×10⁻¹²M, ≤7×10⁻¹²M, 6×10⁻¹²M, ≤5×10⁻¹²M, ≤4×10⁻¹²M, ≤3×10⁻¹²M, ≤2×10⁻¹² M, ≤1×10⁻¹² M or lower).

The term “specific binding” or “specifically binds” as used herein refers to a non-random binding reaction between two molecules, such as for example between an antibody and an antigen. Specific binding can be characterized in binding affinity, A K_{D} value of 10⁻⁶ M (e.g., ≤5×10⁻⁷ M, ≤2×10⁻⁷ M, ≤10⁻⁷ M, ≤5×10⁻⁸ M, 2×10⁻⁸ M, ≤10⁻⁸ M, ≤5×10⁻⁹ M, ≤4×10⁻⁹M, 3×10⁻⁹ M, ≤2×10⁻⁹ M, or ≤10⁻⁹ M and any value 10⁻⁶) can indicate specific binding between a binding agent (e.g., antibody such as single domain antibody) and TROP2 (e.g., human TROP2).

As used herein, the term “competing for binding”, “compete for binding”, “compete(s) with” or “competing with” refers to the ability of a first antibody or antigen-binding fragment thereof (e.g., single domain antibody or antigen binding fragment thereof) to inhibit the binding interaction between TROP2 and a second anti-TROP2 antibody (e.g., a single domain antibody or antigen binding fragment thereof) to any detectable degree.

As used herein the term “epitope” as used herein refers to the specific group of atoms or amino acid residues on an antigen to which an antibody (e.g., single domain antibody) binds. Two antibodies may bind the same or a closely related epitope within an antigen if they exhibit competitive binding for the antigen. An epitope can be linear or conformational (i.e., including amino acid residues spaced apart). For example, if an antibody or antigen binding fragment blocks binding of a reference antibody (e.g., single domain antibody) to the antigen by at least 85%, or at least 90%, or at least 95%, then the antibody or antigen-binding fragment may be considered to bind the same/closely related epitope as the reference antibody. For example, a monoclonal, chimeric, human or humanized antibody or an antigen binding fragment thereof may compete with a single domain antibody of the present disclosure for binding to human TROP2.

As used herein, the term “sequence identity” of the present invention indicates the degree of identity between two nucleic acid or two amino acid sequences when best compared and compared when a mutation such as substitution, insertion or deletion is appropriate. The sequence identity can be at least 85%, 90% or 95%, preferably at least 95%. Non-limiting examples include 85%, 86%, 87%, 88%, 89%, 90%, 91%, 92%, 93%, 94%, 95%, 95%, 96%, 97%, 98%, 99%, and 100%.

The term “antigen binding fragment(s)” as used in the expression “antibody or antigen binding fragment thereof” or “single domain antibody or antigen binding fragment thereof” refers to a fragment of the antibody or single antibody that encompasses the antigen binding domain and that may incorporate or not, other portion(s) of the antibody or single domain antibody such as for example amino acid residues of the hinge region, amino acid residues of a constant region, portion of a Fc region. Regardless of structure, an antigen binding fragment binds to the same antigen that is recognized by the complete antibody (e.g., single domain antibody).

An antigen binding fragment of a single domain antibody includes for example the CDRs and sequences that encompass the CDRs such as the entire variable region or portion thereof or the entire heavy chain or portion thereof and may include or not, other portion(s) of the antibody.

The term “antigen binding domain” relates to the portion of an antibody that is involved in antigen binding and comprises for example, one or more CDRs, one or more framework regions (FR) or the entire variable region. The term “antigen binding domain” in the context of a single domain antibody thereof relates to the portion of single domain antibody that is involved in antigen binding and comprises for example, one or more of CDR1 (CDRH1), CDR2 (CDRH2) or CDR3 (CDRH3), one or more framework regions FR1, FR2, FR3, FR4 or the entire variable region (VH or VHH). The term “antigen binding domain”” in the context of a native antibody thereof relates to the portion of a native antibody that is involved in antigen binding and comprises for example, one or more of CDRH1, CDRH2, CDRH3, CDRL1, CDRL2 or CDRL3, one or more light chain or heavy chain framework regions FR1, FR2, FR3, FR4 or one or both entire variable regions (heavy chain variable region (VH) and/or light chain variable region (VL)).

As used herein the terms “CDRH1”, “CDRH2” and “CDRH3” respectively refer to the CDR1, CDR2 or CDR3 of an antibody heavy chain.

### TROP2 Antigens

Antibodies or antigen binding fragments of the present disclosure may be obtained, for example, by immunizing an animal with a TROP2 antigen.

Alternatively, the antibodies or antigen binding fragments of the present disclosure may be obtained from a library of antibodies.

Binding agents that bind to TROP2 may be identified by in silico, in vitro and/or in vivo methods. Several in vitro methods rely on the binding of candidate antibodies or antigen binding fragments thereof to an antigen. As such anti-TROP2 antibodies or antigen binding fragments thereof may be identified by methods involving binding to the TROP2 antigen disclosed herein and/or using assays disclosed herein.

In an embodiment, the TROP2 antigen comprises a human TROP2 protein (e.g., SEQ ID NO:39) or to a TROP2 homologue or TROP2 variant having a sequence at least 80% identical to the human TROP2 protein thereof or to a portion of human TROP2 protein (e.g., an extracellular domain of human TROP2: SEQ ID NO:164), of the homologue or variant thereof. In some embodiment, the TROP2 homologue is a TROP2 sequence or portion thereof of a primate.

In an embodiment, the TROP2 antigen is a human TROP2, a TROP2 homologue, a TROP2 variant having a sequence at least 80% identical to human TROP2 thereof or a fragment thereof.

In an exemplary embodiment, the TROP2 homologue is cynomolgus TROP2.

In another exemplary embodiment, the TROP2 homologue is Rhesus TROP2.

In some embodiments, the TROP2 antigen comprises the extracellular domain of TROP2 (e.g., of human TROP2, TROP2 homologue or variant thereof).

In some embodiment, the TROP2 antigen comprises the extracellular domain (ECD) of TROP2 (as represented by amino acids 27 to 274 of TROP2 (e.g., SEQ ID NO:39 or SEQ ID NO:40), see Goldenberg et al., 2018, Ikeda, M. et al., 2015, the entire contents of which are incorporated herein by reference).

In some embodiment, the TROP2 antigen comprises an epitope that is common to human TROP2 and to cynomolgus TROP2.

In some embodiment, the TROP2 antigen comprises an epitope that is similar in human TROP2 and to cynomolgus TROP2.

In some embodiments, the TROP2 antigen comprises amino acid residues 27-274 of human TROP2, or of a TROP2 homologue or a fragment thereof. Accordingly, the TROP2 antigen may comprise a polypeptide comprising a human TROP2 amino acid sequence consisting of amino acid residues 27-274 or a fragment thereof.

In other embodiments, the TROP2 antigen comprises amino acid residues 27-146 of human TROP2, or of a TROP2 homologue or a fragment thereof. Accordingly, the TROP2 antigen may comprise a polypeptide comprising a human TROP2 amino acid sequence consisting of amino acid residues 27-146 or a fragment thereof.

In yet other embodiments, the TROP2 antigen comprises amino acid residues 27-73 of human TROP2, or of a TROP2 homologue or a fragment thereof. Accordingly, the TROP2 antigen may comprise a polypeptide comprising a human TROP2 amino acid sequence consisting of amino acid residues 27-73 or a fragment thereof.

In accordance with the present disclosure, the TROP2 antigen may comprise at least 10 amino acid residues of human TROP2.

More particularly, the TROP2 antigen is a fragment of at least 10 amino acid residues of a human TROP2 amino acid sequence consisting of amino acid residues 27-146.

In accordance with the present disclosure the TROP2 antigen comprises between 10 to 110 amino acid residues, including for example between 10 to 100 amino acid residues, between 10 to 90 amino acid residues, between 10 to 80 amino acid residues, between 10 to 70 amino acid residues, between 10 to 60 amino acid residues, between 10 to 50 amino acid residues, between 10 to 40 amino acid residues, between 10 to 30 amino acid residues, 10 to 29 amino acid residues, between 10 to 28 amino acid residues, between 10 to 27 amino acid residues, between 10 to 26 amino acid residues, between 10 to 25 amino acid residues, between 10 to 24 amino acid residues, between 10 to 23 amino acid residues, between 10 to 22 amino acid residues, between 10 to 21 amino acid residues, between 10 to 20 amino acid residues, between 10 to 19 amino acid residues, between 10 to 18 amino acid residues, between 10 to 17 amino acid residues, between 10 to 16 amino acid residues, between 10 to 15 amino acid residues, between 10 to 14 amino acid residues, between 10 to 13 amino acid residues, between 10 to 12 amino acid residues between 10 to 11 amino acid residues of a human TROP2 amino acid sequence consisting of amino acid residues 27-146.

For instances, the TROP2 antigen comprises at least 10 amino acid residues of a human TROP2 amino acid sequence consisting of amino acid residues 27-73.

In accordance with the present disclosure the TROP2 antigen comprises between 10 to 45 amino acid residues, including for example between 10 to 40 amino acid residues, between 10 to 35 amino acid residues, between 10 to 30 amino acid residues, 10 to 29 amino acid residues, between 10 to 28 amino acid residues, between 10 to 27 amino acid residues, between 10 to 26 amino acid residues, between 10 to 25 amino acid residues, between 10 to 24 amino acid residues, between 10 to 23 amino acid residues, between 10 to 22 amino acid residues, between 10 to 21 amino acid residues, between 10 to 20 amino acid residues, between 10 to 19 amino acid residues, between 10 to 18 amino acid residues, between 10 to 17 amino acid residues, between 10 to 16 amino acid residues, between 10 to 15 amino acid residues, between 10 to 14 amino acid residues, between 10 to 13 amino acid residues, between 10 to 12 amino acid residues between 10 to 11 amino acid residues of a human TROP2 amino acid sequence consisting of amino acid residues 27-73.

In some instances, the TROP2 antigen comprises a human TROP2 amino acid sequence consisting of amino acid residues 27-56 of human TROP2 or a fragment thereof.

In other instances, the TROP2 antigen comprises a human TROP2 amino acid sequence consisting of amino acid residues 27-66 of human TROP2 or a fragment thereof.

In yet other instances, the TROP2 antigen comprises a human TROP2 amino acid sequence consisting of amino acid residues 47-56 of human TROP2 or a fragment thereof.

In other instances, the TROP2 antigen comprises a human TROP2 amino acid sequence consisting of amino acid residues 60-66 of human TROP2 or a fragment thereof.

In yet other instances, the TROP2 antigen comprises a human TROP2 amino acid sequence consisting of amino acid residues 47-66 of human TROP2 or a fragment thereof.

For example, the TROP2 antigen comprises a human TROP2 amino acid sequence consisting of amino acid residues 27-56, 27-66, 28-66, 29-66, 30-66, 31-66, 32-66, 33-66, 34-66, 35-66, 36-66, 37-66, 38-66, 39-66, 40-66, 41-66, 42-66, 43-66, 44-66, 45-66, 46-66 or 47-66.

In some instances, the TROP2 antigen may exclude the cysteine poor (CPD) domain of TROP2.

An exemplary and non-limiting embodiment of a TROP2 antigen is provided in SEQ ID NO:164.

Accordingly, in some embodiment, the binding agent, or antigen binding domain(s) of the binding agent of the present disclosure is capable of binding human TROP2.

In some embodiment, the binding agent, or antigen binding domain(s) of the binding agent of the present disclosure is capable of binding human TROP2 and cynomolgus TROP2.

In some embodiment, the binding agent, or antigen binding domain(s) of the binding agent of the present disclosure preferentially binds to human TROP2 over mouse TROP2 or has a better affinity for human TROP2 over mouse TROP2.

In some embodiment, the binding agent, or antigen binding domain(s) of the binding agent of the present disclosure preferentially binds to human TROP2 over rat TROP2 or has a better affinity for human TROP2 over rat TROP2.

In some embodiment, the binding agent, or antigen binding domain(s) of the binding agent of the present disclosure binds to amino acid residues that are common to the extracellular domain of human TROP2 and to the extracellular domain of cynomolgus TROP2.

In some embodiment, the binding agent, or antigen binding domain(s) of the binding agent of the present disclosure binds to amino acid residues that are not present in the extracellular domain of mouse TROP2.

In some embodiment, the binding agent, or antigen binding domain(s) of the binding agent of the present disclosure binds to amino acid residues that are not present in the extracellular domain of rat TROP2.

In some embodiment, the binding agent, or antigen binding domain(s) of the binding agent of the present binds to amino acid residues that are present in the extracellular domain of human TROP2, cynomolgus TROP2, mouse TROP2 and rat TROP2.

Accordingly, the binding agent, or antigen binding domain(s) of the binding agent may be capable of binding to human TROP2 and to cynomolgus TROP2.

In exemplary embodiments, the binding agent, or antigen binding domain(s) of the binding agent may be capable of binding to a denatured form human TROP2.

In another exemplary embodiments, the binding agent, or antigen binding domain(s) of the binding agent may be capable of binding to a reduced form of human TROP2.

In yet other exemplary embodiments, the binding agent, or antigen binding domain(s) of the binding agent may be capable of binding to a deglycosylated form of human TROP2.

Binding agents Exemplary embodiments of binding agents of the present disclosure includes a polypeptide chain disclosed herein.

Exemplary embodiments of binding agents of the present disclosure includes antibodies or antigen binding fragments thereof.

Exemplary embodiments of binding agents therefore include without limitations, native antibodies or antigen binding fragments thereof or single domain antibodies or antigen binding fragments thereof or variants thereof such as for example, humanized, chimeric or human antibodies or antigen binding fragments thereof or humanized or chimeric single domain antibodies or antigen binding fragments thereof.

Single domain antibodies or antigen binding fragments thereof disclosed herein are particularly contemplated as well as binding agents that comprise one or more antigen binding domain(s) of one or more single domain antibodies disclosed herein.

Single domain antibodies that compete with one or more single domain antibodies (having an amino acid sequence as disclosed herein) for the binding of a TROP2 antigen are also encompassed by the present disclosure.

Other exemplary embodiments of binding agents of the present disclosure include binding agents comprising antigen binding domain(s) such as antibody-like molecules (including single chain antibody), protein scaffold molecules and immune cell modulating agents.

Accordingly, exemplary embodiments of binding agents of the present disclosure include antibody-like molecules that comprise one or more antigen binding domain(s) of one or more single antibodies disclosed herein.

Additional exemplary embodiments of binding agents of the present disclosure include protein scaffold molecules that comprise one or more antigen binding domain(s) of one or more single antibodies disclosed herein.

Yet additional exemplary embodiments of binding agents of the present disclosure comprises immune cell modulating agents that comprise one or more antigen binding domain(s) of one or more single antibodies disclosed herein.

Binding agents including for example and without limitations, antibodies or antigen binding fragments thereof as well as single domain antibodies or antigen binding fragments thereof that competes with of a single domain antibody having an amino acid sequence disclosed herein are also encompassed by the present disclosure.

The present disclosure thus relates to a binding agent comprising one or more antigen binding domains, wherein at least one of the one or more antigen binding domains comprise an antigen binding fragment thereof that is capable of binding to trophoblast cell surface antigen-2 (TROP2).

In some embodiments, the binding agent of the present disclosure is capable of binding to the extracellular domain (ECD) of TROP2.

In some embodiments, the binding agent of the present disclosure is capable of binding to the cysteine-rich domain and/or to the thyroglobulin type-1 domain of TROP2.

In some embodiments, the binding agent of the present disclosure is capable of binding to an epitope comprising amino acids of the extracellular domain of TROP2.

In some embodiments, the binding agent of the present disclosure is capable of binding to an epitope comprising amino acids of the cysteine-rich domain and/or of the thyroglobulin type-1 domain of TROP2.

In some embodiments, the binding agent of the present disclosure is capable of binding to an epitope including amino acid residues comprised within amino acid sequence 27-274 of TROP2 or a fragment thereof.

In some embodiments, the binding agent of the present disclosure is not capable of binding to the cysteine-poor domain (CPD) of TROP2.

In some embodiments, the binding agent of the present disclosure is not capable of binding to the amino acid 146 to 274 of TROP2.

In some embodiments, the binding agent comprises an antigen binding domain of a single domain antibody capable of binding TROP2.

In some embodiments, the binding agent comprises a single domain antibody.

In other embodiments, the binding agent is a single domain antibody.

In some embodiments, the antigen binding domain of the binding agent comprises the CDRs of a single domain antibody.

In some embodiments, the antigen binding domain of the binding agent is a variable region of a single domain antibody. In some instances, the variable region may be a camelid VH or VHH. In some instances, the variable region comprises for example, camelid CDRs and framework regions such as camelid framework regions, humanized framework regions, human framework regions, mouse framework regions.

In some embodiments, the binding agents comprises a variable region that comprises CDRs of camelid origin and humanized framework regions.

In other embodiments, the binding agents comprises a variable region that comprises CDRs of camelid origin and human framework regions.

In some embodiments the binding agent comprises two heavy chain variable regions of a single domain antibody each comprising a dimerization domain thereby forming a dimer.

In some embodiments, the level of humanization of a single domain antibody may be calculated based on the sequence identity to the closest human VH germline gene. Accordingly, the percentage of humanization score is based on framework regions 1-3 (FR1, FR2, FR3) and CDR1 and CDR2. In accordance with an embodiment, the sequence of CDR3 and FR4 are not accounted for assessing the level of humanization of a single domain disclosed herein.

In exemplary embodiments, the level of humanization of the antigen binding domain is at least 80%.

In other exemplary embodiments, the level of humanization of the variable region of the single domain antibody of the present disclosure is at least 80%.

In some embodiments, the binding agent of the present disclosure may comprise one or more polypeptide chains each independently comprising in a N- to C-terminal fashion an amino acid sequence of formula I:

X-[(Abₐ)-(L_{b})]ₘ-(DD)-[(Lc)-(Abd)]ₙ-Y
Wherein m is 0, 1, 2 or an integer greater than 2;
Wherein n is 0, 1, 2 or an integer greater than 2;
Wherein m and n are not 0 simultaneously;
Wherein Abₐ, Ab_{d}, each represents an antigen binding domain wherein at least one of the antigen binding domains is an antigen binding domain 1 (ABD1), an antigen binding domain 2 (ABD2) or an antigen binding domain 3 (ABD3);
Wherein X or Y are independently present or absent and comprises an amino acid sequence;
Wherein L_{b}, L_{c}, each independently comprises one or more linkers; and
Wherein DD represents a dimerization domain.

In some embodiments the binding agent comprises one or more polypeptide chains wherein at least one of the polypeptide chains comprises formula II: X-(Abₐ₁)-(L_{b1})-(DD)-(L_{c1})-(Ab_{d1})-Y (formula II).

In some embodiments the binding agent comprises one or more polypeptide chains wherein at least one of the polypeptide chains comprises formula III: X-(Abₐ₁)-(L_{b1})-(DD)-(L_{c1})-(Ab_{d1})-(L_{c2})-(Ab_{d2})-Y (formula III).

In some embodiments the binding agent comprises one or more polypeptide chains wherein at least one of the polypeptide chains comprises formula IV: X-(Abₐ₁)-(L_{b2})-(Abₐ₂)-(L_{b1})-(DD)-(L_{c1})-(Ab_{d1})-Y (formula IV).

In some embodiments the binding agent comprises one or more polypeptide chains wherein at least one of the polypeptide chains comprises formula V: X-(Abₐ₁)-(L_{b2})-(Abₐ₂)-(L_{b1})-(DD)-(L_{c1})-(Ab_{d1})-(L_{c2})-(Abd2)-Y (formula V).

In some embodiments the binding agent comprises one or more polypeptide chains wherein at least one of the polypeptide chains comprises formula VI: X-(Abₐ₁)-(L_{b2})-(Abₐ₂)-(L_{b1})-(DD)-(L_{c1})-(Ab_{d1})-(L_{c2})-(Abd2)-(L_{c3})-(Abd3)-Y (formula VI).

In some embodiments the binding agent comprises one or more polypeptide chains wherein at least one of the polypeptide chains comprises formula VII: X-(Abₐ₁)-(L_{b3})-(Abₐ₂)-(L_{b2})-(Abₐ₃)-(L_{b1})-(DD)-(L_{c1})-(Ab_{d1})-(L_{c2})-(Abd2)-Y (formula VII).

In some embodiments the binding agent comprises one or more polypeptide chains wherein at least one of the polypeptide chains comprises formula VIII: X-(Abₐ₁)-(L_{b3})-(Abₐ₂)-(L_{b2})-(Abₐ₃)-(L_{b1})-(DD)-(L_{c1})-(Ab_{d1})-(L_{c2})-(Abd2)-(L_{c3})-(Abd3)-Y (formula VIII).

In some embodiments Abₐ₁, Abₐ₂, Abₐ₃, Ab_{d1}, Ab_{d2}, or Ab_{d3}, each independently comprises an antigen binding domain.

In some embodiments L_{b1} comprises a linker or linkers and/or a hinge region of an antibody or antigen binding fragment thereof. In some embodiments, the hinge region is a hinge region of a naturally occurring antibody. In exemplary embodiments, the hinge may comprise an amino acid sequence as set forth in SEQ ID NO:36, SEQ ID NO:42, SEQ NO:481 or a portion thereof. In other embodiments, the hinge region is a mutated or synthetic hinge region as disclosed herein. In some embodiments, the mutated or synthetic hinge may have a sequence at least 80%, at least 85%, at least 90%, at least 95%, at least 96%, at least 87%, at least 98%, at least 99% identical to a hinge of a naturally occurring antibody. In an exemplary embodiment, the mutated or synthetic hinge may have a sequence as set fort in SEQ ID NO:481 or a portion thereof.

In some embodiments, the hinge region may be replaced by a linker or spacer. In some embodiments, the hinge region, linker or spacer are optional.

In some embodiments L_{b2}, L_{b3} L_{c1}, L_{c2}, and L_{c3} each independently comprise a linker or linkers as disclosed herein.

Dimerization domain and linkers In accordance with the present disclosure, the binding agent may be composed of two polypeptide chains each comprising one or more antigen binding domains and a dimerization domain. The one or more antigen binding domains may be separated by linkers.

Exemplary embodiments of dimerization domains and linkers are provided in PCT/CA2020/051753 filed on Dec. 18, 2020, and published on Jun. 24, 2021, under No. WO2021/119832A1, the entire content of which is incorporated herein by reference.

In some embodiments, the dimerization domain allows the formation of homodimers.

In some embodiments, the dimerization domain allows the formation of heterodimers.

In some embodiments, the dimerization domain comprises an amino acid sequence that is contiguous to the amino acid sequence of the antigen binding domain.

In some embodiments, the dimerization domain comprises an immunoglobulin dimerization domain. Other dimerization domains known to a person skilled in the art are contemplated herein including leucine zippers, etc.

In some embodiments, the dimerization domain comprises an IgG, IgM, IgA, IgD or IgE dimerization domain (from human or animal IgGss, IgM, IgAs, IgDs or IgEs).

In some embodiments, the dimerization domain comprises a constant region of an antibody or a portion thereof.

In some embodiments, the constant region or portion thereof is from an IgG1, an IgG2, an IgG3 or an IgG4 heavy chain or combination thereof. For example, the constant region or portion thereof comprises amino acid sequence of an IgG1, an IgG2, an IgG3 or an IgG4 heavy chain or combination thereof. In some instances the amino acid sequence of the constant region or portion thereof (e.g., CH3 domain) is at least 80% identical to the sequence of a naturally occurring IgG1, an IgG2, an IgG3 or an IgG4 heavy chain.

In some embodiments, the constant region or portion thereof is from a human IgG1 heavy chain.

In some embodiments, the constant region or portion thereof is from a human IgG2 heavy chain.

In some embodiments, the constant region or portion thereof is from a human IgG3 heavy chain.

In some embodiments, the constant region or portion thereof is from a human IgG4 heavy chain.

In some embodiments, the binding agent comprises a Fc region of an antibody or a portion thereof.

In some embodiments, the binding agent comprises a Fc region or a portion thereof that comprises Fc modification(s) or not.

In an exemplary embodiment, the binding agent may comprise a Fc or a portion thereof that is capable of binding to an Fc receptor of monocytes and/or macrophages.

In another exemplary embodiment, the binding agent may comprise a Fc or a portion thereof that is capable of binding to an Fc receptor on CD68+ macrophages.

In other embodiments, the binding agent comprises an altered or mutated Fc region or portion thereof. For example, the Fc region or portion thereof may be altered or mutated so as to modify one or more characteristics of the binding agent. In exemplary embodiments, the Fc region or portion thereof is unglycosylated. In other exemplary embodiments, the Fc region or portion thereof comprises one or more of the Fc modification(s) of Table A.

In other exemplary embodiments, the Fc region or portion thereof is altered or mutated so as to increase ADCC activity. For example, afucosylation (e.g., N297 in accordance with the EU numbering system) may result in an increased FcRgII binding on NK cells and may potently increase ADCC. Accordingly, in some embodiments, the Fc region or portion thereof may have a reduced number of fucose residues. In other embodiments, the binding agent comprises an afucosylated Fc region or portion thereof. In yet other embodiments, the Fc region or portion thereof of the binding agent lacks fucose residues. In additional embodiments, the Fc region or portion thereof of the binding agent lacks core fucose residues. In further embodiments, the Fc region or portion thereof of the binding agent fully lacks core fucose residues.

In some embodiments, the dimerization domain comprises a CH3 domain.

In some embodiments, the CH3 domain is a natural CH3 domain or a mutated CH3 domain.

In some embodiments, the dimerization domain also comprises a CH2 domain.

In some embodiments, the dimerization domain comprises a CH3 domain of an antibody.

The dimerization domain may also comprise a CH2 domain of an antibody.

In some embodiments, the CH2 domain is a natural CH2 domain or a mutated CH2 domain.

In some embodiments, the dimerization domain comprises a natural CH2 domain and a natural CH3 domain.

In some embodiments, the dimerization domain comprises a natural CH2 domain and a mutated CH3 domain.

In some embodiments, the dimerization domain comprises a mutated CH2 domain and a natural CH3 domain.

In some embodiments, the dimerization domain comprises a mutated CH2 domain and a mutated CH3 domain.

In some embodiments, the CH3 domain may have a sequence as set forth in SEQ ID NO:38 or a sequence at least 80% identical, at least 85% identical, at least 90% identical, at least 99% identical thereof.

In other embodiments, the CH3 domain may have a sequence as set forth in SEQ ID NO:43 or a sequence at least 80% identical, at least 85% identical, at least 90% identical, at least 99% identical thereof.

In some embodiments, the CH2 domain may have a sequence as set forth in SEQ ID NO:37 or a sequence at least 80% identical, at least 85% identical, at least 90% identical, at least 99% identical thereof.

In some embodiments, the CH2 domain may have a sequence as set forth in SEQ ID NO:42 or a sequence at least 80% identical, at least 85% identical, at least 90% identical, at least 99% identical thereof.

In some embodiments, the dimerization domain may comprise a CH3 domain having a sequence as set forth in SEQ ID NO:38 or a sequence at least 80% identical, at least 85% identical, at least 90% identical, at least 99% identical thereof and a CH2 domain having a sequence as set forth in SEQ ID NO:37 or as set forth in SEQ ID NO:42 or a sequence at least 80% identical, at least 85% identical, at least 90% identical, at least 99% identical thereof.

In some embodiments, the dimerization domain may comprise a CH3 domain having a sequence as set forth in SEQ ID NO:43 or a sequence at least 80% identical, at least 85% identical, at least 90% identical, at least 99% identical thereof and a CH2 domain having a sequence as set forth in SEQ ID NO:37 or as set forth in SEQ ID NO:42 or a sequence at least 80% identical, at least 85% identical, at least 90% identical, at least 99% identical thereof.

In some embodiments, the dimerization domain may comprise the sequence set forth in SEQ ID NO: 212 or a sequence at least 80% identical, at least 85% identical, at least 90% identical, at least 99% identical thereof.

In some embodiments, the dimerization domain may comprise the sequence set forth in SEQ ID NO: 506 or a sequence at least 80% identical, at least 85% identical, at least 90% identical, at least 99% identical thereof.

In some embodiments, the dimerization domain may comprise the sequence set forth in SEQ ID NO: 214 or a sequence at least 80% identical, at least 85% identical, at least 90% identical, at least 99% identical thereof.

In some embodiment, the dimerization domain comprises a hinge region such as a hinge region of a naturally occurring antibody or antigen binding fragment thereof or a mutated or synthetic hinge region.

In some embodiment, the dimerization domain comprises a peptidic linker.

In some embodiments, the linker is an amino acid sequence of at least 5, 10, 15, 20, 25, 30, 35, 40, 45 or 50 amino acid residues in length.

In some embodiment, the linker is non-cleavable.

In some embodiments, the linker comprises one or more units of GGGGS (SEQ ID NO:206).

In some embodiments, the linker may comprise the formula (X(PAPAP))IKA (SEQ ID NO:207) wherein n is an integer selected from 1 to 10, wherein X is present or absent and is A. In some embodiments, the linker may comprise the sequence set forth in SEQ ID NO:163.

In some embodiments, the linker is an antibody hinge or a portion thereof. For example, in some embodiments, the linker may comprise human IgG1 hinge sequence or a portion thereof. In some embodiments, the linker may comprise human IgG2 hinge sequence or a portion thereof. In some embodiments, the linker may comprise human IgG3 hinge sequence or a portion thereof. In yet other embodiment, the linker may comprise human IgG4 hinge sequence or a portion thereof.

### Antibodies and Antigen Binding Fragments

In some embodiments, the binding agent is an anti-TROP2 antibody or an antigen binding fragment thereof. It is to be understood herein that the following represent exemplary and non-limiting embodiments of antibodies or antigen binding fragments thereof.

Antibodies or antigen binding fragments thereof (or their sequence) may be obtained from immunization of an animal (including a camel, a llama, a dromedary, an alpaca, a transgenic animal) with a TROP2 antigen as disclosed herein. For example, single domain antibodies or antigen binding fragments thereof (or their sequence) may be obtained by immunizing an animal such as a dromedary, a camel, a llama, an alpaca, a rabbit with a TROP2 antigen (e.g., ECD or fragments thereof). Alternatively, the sequence of a single domain antibody or antigen binding domain thereof may be obtained by immunizing a transgenic animal that is able to express single domain antibodies (see international application No. PCT/CA2021/050951 filed on Jul. 12, 2021, and published on Jan. 20, 2022, under No. WO2022/011457 A1 the entire content of which is incorporated herein by reference).

In some instances, an antibody or antigen binding fragment thereof (or its sequence) is obtained from serum or tissue of immunized animals such as bone marrow, spleen and/or lymph nodes.

An hybridoma that results from fusion of cells of an immunized animal with a malignant and immortal plasma cell such as myeloma cells lacking hypoxanthine-guanine-phosphoribosyltransferase (HGPRT) an enzyme allowing cells to grow in HAT medium (hypoxanthine-aminopterin-thymidine) may be generated. Spleen cells are particularly useful to generate hybridomas. The antibody or antigen binding fragment thereof may be isolated from an hybridoma.

The nucleic acid sequence of an antibody or antigen binding fragment thereof may be obtained by PCR amplification and sequencing from cells or tissue able to express the antibody or antigen binding fragment thereof. The amino acid sequence information is obtained from the nucleic acid sequence. Due to inherent degeneracy of the genetic code, a nucleic acid molecule encoding the same amino acid sequence but having a different nucleic acid sequence compared to the original nucleic acid sequence may be generated.

Once a nucleic acid sequence encoding the amino acid sequence of an antibody is obtained, recombinant expression of an antibody is rendered possible by transfection of one or more expression vector(s) into immortalized mammalian cell lines.

Alternatively, the sequence of an antibody of antigen binding fragment thereof may also be obtained by screening an established antibody library for sequences that have desired features such as for example, homology or identity with a reference antibody.

Antibodies of the present disclosure includes native or intact antibodies that specifically bind to TROP2. A native or intact antibody includes an antibody that is recombinantly expressed, isolated from immunized animals or from an hybridoma.

Exemplary embodiments of binding agents of the present disclosure include antibodies or antigen binding fragments thereof that specifically bind to at least one epitope comprising amino acids of the extracellular domain (ECD) of TROP2. Other exemplary embodiments of binding agents of the present disclosure include antibodies or antigen binding fragments thereof that specifically bind to at least one epitope comprising amino acids of the cysteine rich domain of TROP2. In some embodiments, the antibodies or antigen binding fragments thereof bind to a TROP2 antigen disclosed herein.

In an embodiment, the antibodies of the present disclosure specifically bind to human TROP2. In another embodiment, the antibodies of the present disclosure specifically bind to cynomolgus TROP2. In another embodiment, the antibodies of the present disclosure specifically bind to Rhesus TROP2.

More particularly, the antibodies of the present disclosure specifically bind to the ECD or the CRD of human TROP2.

A native or intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including CDRL1, CDRL2, and CDRL3, heavy chain CDRs including CDRH1, CDRH2, CDRH3). CDR boundaries for the antibodies and antigen-binding fragments disclosed herein may be defined or identified by the conventions of Kabat, IMGT, Chothia, or Al-Lazikani. The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgAQ1 (alpha1 heavy chain), or IgA2 (alpha2 heavy chain).

Antibodies of the present disclosure includes chimeric antibodies that specifically bind to TROP2. A chimeric antibody is a recombinant protein that contains the variable region(s) including the complementarity determining regions (CDRs) of an antibody derived from one or more species, such as a rodent antibody, while the constant domains of the antibody molecule are derived from those of another species, such as a human antibody.

Antibodies of the present disclosure includes humanized antibodies that specifically bind to TROP2. A humanized antibody is a recombinant protein in which the CDRs or CDR residues from an antibody from one species; e.g., a rodent antibody, are transferred from the heavy and light variable chains of the rodent antibody into human heavy and light variable regions (e.g., framework region sequences). The constant domains of the antibody molecule are derived from those of a human antibody. In certain embodiments, a limited number of framework region amino acid residues from the parent (rodent) antibody may be substituted into the human antibody framework region sequences. In other examples, as simply transferring mouse CDRs into human FRs often results in a reduction or even loss of antibody affinity, additional modification might be required in order to restore the original affinity of the murine antibody. This can be accomplished by the replacement of one or more some human residues in the FR regions with their murine counterparts to obtain an antibody that possesses good binding affinity to its epitope. In some embodiment the humanized antibody is a humanized single domain antibody. In some embodiments, a humanized antibody that competes with a single domain antibody disclosed herein is encompassed by the present disclosure.

Antibodies of the present disclosure includes human antibodies that specifically binds to TROP2. A fully human antibody can be obtained from a transgenic non-human animal. Methods for producing fully human antibodies using either combinatorial approaches or transgenic animals transformed with human immunoglobulin loci are known in the art. In one alternative, the phage display technique may be used to generate human antibodies. The fully human antibodies are expected to exhibit even fewer side effects than chimeric or humanized antibodies and to function in vivo as essentially endogenous human antibodies. In some embodiments, a human antibody that competes with a single domain antibody disclosed herein is encompassed by the present disclosure.

Antibodies of the present disclosure includes single domain antibodies (sdAbs) that specifically binds to TROP2. As used herein the expressions “single domain antibody” and “heavy chain only antibody” are used interchangeably. The term “single domain antibody” is used in its broad sense and includes naturally occurring single domain antibodies or variants thereof such as, without limitations, humanized single domain antibodies or chimeric single domain antibodies. A single domain antibody is said to be chimeric when it possesses a constant region of another species. In an embodiment, the single domain antibody is a chimeric single domain antibody. In some embodiments, the chimeric single domain antibody comprises a humanized variable region and a human constant region. In another embodiment, the chimeric single domain antibody comprises the variable region of a native single domain antibody. The term “single domain antibody” also encompass mutated versions of naturally occurring single domain antibodies, mutated versions of humanized single domain antibodies as well mutated versions of chimeric single domain antibodies in which the amino acid sequence comprises amino acid substitutions, deletions and/or additions.

A single domain antibody (sdAb) is usually composed of two heavy chains. A single domain antibody is about 12-15 kDa in size (about 110 amino acids in length). A sdAb can selectively bind to target antigens, like full-size antibodies, and have similar affinities for antigens. However, because of their much smaller size, they may be capable of better penetration into solid tumors. The smaller size also contributes to the stability of the sdAb, which is more resistant to pH and temperature extremes than full size antibodies (Van Der Linden et al., 1999, Biochim Biophys Act 1431:37-46). Single-domain antibodies were originally developed following the discovery that camelids (camels, alpacas, llamas) possess fully functional antibodies without light chains (e.g., Hamsen et al., 2007, Appl Microbiol Biotechnol 77:13-22). Naturally occurring single domain antibodies consist of a single variable region (VH or VHH) and two constant domains (CH2 and CH3). The variable region of naturally occurring single domain antibodies may be cloned and fused to a constant region of a naturally occurring antibody heavy chain. In some embodiments, the Fc domain of a naturally occurring single domain antibody is replaced with a Fc or a Fc portion thereof of a human IgG antibody. In some embodiments, the heavy chain constant region of a naturally occurring single domain antibody is replaced with that of a human IgG heavy chain constant region which comprise CH1, CH2 and CH3 domains and optionally CH4 domain and may optionally comprise a hinge region. In some embodiments, the heavy chain constant region of a naturally occurring single domain antibody is replaced with a CH3 or CH2-CH3 domain of a human IgG heavy chain and may optionally comprise a hinge region.

The antigen binding property of a single domain antibody is conferred by the heavy chain variable region. As such, a single heavy chain or even a single heavy variable region (VH or VHH) may be sufficient for specific binding to an antigen. However, a single domain antibody is usually composed of two heavy chains. The two heavy chains may be assembled via a dimerization domain such as for example a constant region of a class G human immunoglobulin. The two heavy chains may also be expressed as a single polypeptide chain.

Accordingly, in some embodiments, binding agents include antibodies and antigen-binding fragments thereof such as, for example and without limitations, single domain antibodies from camelids or sharks, human antibodies including IgGs (including human IgG1, human IgG2, human IgG3, human IgG4), human IgMs, human IgAs (including human IgAQ1 and human IgA2), human IgEs, human IgDs, animal antibodies including for example, IgGs (IgG1, IgG2a, IgG2b, IgG2c, IgG3, IgG4), IgMs, IgAs, IgEs and IgDs and variants (e.g., chimeric, humanized or human) having a similar format.

In other embodiments, binding agents of the present disclosure encompass antigen-binding fragments such as, for example and without limitations, Fab, Fab′, F(ab′)₂, complementarity determining regions, variable regions including VHs, VHHs, VLs, and the like.

An antigen binding fragment can be prepared by known techniques such as by proteolytic hydrolysis of a full-length antibody or by expression in E. coli or another host of the DNA coding for the fragment. An antigen binding fragment can be obtained by pepsin or papain digestion of full-length antibodies by conventional methods. For example, antigen binding fragment can be produced by enzymatic cleavage of antibodies with pepsin to provide an approximate 100 kD fragment denoted F(ab′)₂. A F(ab′)₂ fragment can be further cleaved using a thiol reducing agent, and optionally a blocking group for the sulfhydryl groups resulting from cleavage of disulfide linkages, to produce an approximate 50 Kd Fab′ monovalent fragment. Alternatively, an enzymatic cleavage using papain produces two monovalent Fab fragments and an Fc fragment directly.

In some embodiments, antibodies that binds to TROP2 can be bispecific or multispecific. A “multispecific antibody” is an antibody that can bind simultaneously to at least two targets that are of different structure, e.g., two or more different antigens, two or more different epitopes on the same antigen, or a hapten and/or an antigen or epitope. A “multivalent antibody” is an antibody that can bind simultaneously to at least two targets that are of the same or different structure. Valency indicates how many antigen binding domains the antibody has; i.e., monovalent, bivalent, trivalent or multivalent. Multivalency provides an antibody with an advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a native antibody (e.g., an IgG) is bivalent because it has two antigen binding domains (each binding domain being composed of amino acid residues from the light chain and heavy chain variable regions) but is monospecific because each antigen binding domain binds to the same epitope. Multispecific, multivalent antibodies include antibodies that have more than one antigen binding domain of different specificity. A “bispecific antibody” is an antibody that can bind simultaneously to two targets which are of different structure. Bispecific antibodies (bsAb) and bispecific antibody fragments (bsFab) may have at least antigen binding domain that specifically binds to TROP2.

In addition to recombinant technologies, numerous methods to produce bispecific or multispecific antibodies are known, as disclosed, for example, in U.S. Pat. No. 7,405,320, the Examples section of which is incorporated herein by reference. Bispecific antibodies can be produced by the quadroma method, which involves the fusion of two different hybridomas, each producing a monoclonal antibody recognizing a different antigenic site. Another method for producing bispecific antibodies uses heterobifunctional cross-linkers to chemically tether two different monoclonal antibodies.

### Single Domain Antibodies

An exemplary embodiment of binding agent of the present disclosure is a single domain antibody. It is to be understood herein that the following represent exemplary and non-limiting embodiments of single domain antibodies or antigen binding fragments thereof.

The single domain antibody possesses unique binding features. For example, the complementarity determining region 3 (CDR3) of the sdAb disclosed herein forms a loop fitting into a groove within the cysteine rich domain (CRD) of the extracellular domain (ECD) of TROP2. Amino acid residues of the CDR3 and of the framework region 2 (FR2) interact with amino acid residues of TROP2 cysteine rich domain and together form a clip around the CRD. These unique properties provide a high affinity of sdAbs for TROP2 and a slow dissociation rate. Surprisingly, amino acid residues of the complementarity determining region 1 (CDR1) and of the complementarity determining region 2 (CDR2) do not appear to interact with amino acid residues of the CRD.

The single domain antibodies of the present disclosure also possess anti-cancer activity such as antibody-dependent cellular cytotoxicity (ADCC) and/or antibody-dependent cellular phagocytosis (ADCP) activity. The single domain antibodies of the present disclosure may also be conjugated for therapeutic, or detection uses.

Exemplary embodiments of binding agents of the present disclosure include single domain antibodies or antigen binding fragments thereof that specifically bind to at least one epitope comprising amino acids of the extracellular domain (ECD) of TROP2. Other exemplary embodiments of binding agents of the present disclosure include single domain antibodies or antigen binding fragments thereof that specifically bind to at least one epitope comprising amino acids of the cysteine rich domain of TROP2. In some embodiments, the single domain antibodies or antigen binding fragments thereof bind to a TROP2 antigen disclosed herein.

In an embodiment, the single domain antibodies of the present disclosure specifically bind to human TROP2. In another embodiment, the single domain antibodies of the present disclosure specifically bind to cynomolgus TROP2. In another embodiment, the single domain antibodies of the present disclosure specifically bind to Rhesus TROP2.

More particularly, the single domain antibodies of the present disclosure specifically bind to the ECD or the CRD of human TROP2.

In an exemplary embodiment, the single domain antibody or antigen binding fragment thereof comprises the amino acid sequence of the CDR3 set forth in SEQ ID NO:54 and the amino acid sequence of the FR2 set forth in SEQ ID NO:116 and optionally the amino acid sequence of the CDR1 set forth in SEQ ID NO:52 and/or the amino acid sequence of the CDR2 set forth in SEQ ID NO:53.

In an exemplary embodiment, the single domain antibody or antigen binding fragment thereof comprises the amino acid sequence of the CDR3 set forth in SEQ ID NO:48 and the amino acid sequence of the FR2 set forth in SEQ ID NO:116 and optionally the amino acid sequence of the CDR1 set forth in SEQ ID NO:55 and/or the amino acid sequence of the CDR2 set forth in SEQ ID NO:47.

In an exemplary embodiment, the single domain antibody or antigen binding fragment thereof comprises the amino acid sequence of the CDR3 set forth in SEQ ID NO:220 and the amino acid sequence of the FR2 set forth in SEQ ID NO:116 and optionally the amino acid sequence of the CDR1 set forth in SEQ ID NO:218 and/or the amino acid sequence of the CDR2 set forth in SEQ ID NO:219.

In an exemplary embodiment, the single domain antibody or antigen binding fragment thereof comprises the amino acid sequence of the CDR3 set forth in SEQ ID NO:220 and the amino acid sequence of the FR2 set forth in SEQ ID NO:117 and optionally the amino acid sequence of the CDR1 set forth in SEQ ID NO:218 and/or the amino acid sequence of the CDR2 set forth in SEQ ID NO:219.

In an exemplary embodiment, the single domain antibody or antigen binding fragment thereof comprises the amino acid sequence of the CDR3 set forth in SEQ ID NO:490 and the amino acid sequence of the FR2 set forth in SEQ ID NO:505 and optionally the amino acid sequence of the CDR1 set forth in SEQ ID NO:488 and/or the amino acid sequence of the CDR2 set forth in SEQ ID NO:489.

In an exemplary embodiment, the single domain antibody or antigen binding fragment thereof comprises the amino acid sequence of the CDR3 set forth in SEQ ID NO:501 and the amino acid sequence of the FR2 set forth in SEQ ID NO:116 and optionally the amino acid sequence of the CDR1 set forth in SEQ ID NO:500 and/or the amino acid sequence of the CDR2 set forth in SEQ ID NO:489.

In an exemplary embodiment, the single domain antibody or antigen binding fragment thereof comprises the amino acid sequence of the CDR3 set forth in SEQ ID NO:230 and the amino acid sequence of the FR2 set forth in SEQ ID NO:116 and optionally the amino acid sequence of the CDR1 set forth in SEQ ID NO:228 and/or the amino acid sequence of the CDR2 set forth in SEQ ID NO:229.

In an exemplary embodiment, the single domain antibody or antigen binding fragment thereof comprises the amino acid sequence of the CDR3 set forth in SEQ ID NO:230 and the amino acid sequence of the FR2 set forth in SEQ ID NO:361 and optionally the amino acid sequence of the CDR1 set forth in SEQ ID NO:228 and/or the amino acid sequence of the CDR2 set forth in SEQ ID NO:229.

In an exemplary embodiment, the single domain antibody or antigen binding fragment thereof comprises the amino acid sequence of the CDR3 set forth in SEQ ID NO:238 and the amino acid sequence of the FR2 set forth in SEQ ID NO:116 and optionally the amino acid sequence of the CDR1 set forth in SEQ ID NO:236 and/or the amino acid sequence of the CDR2 set forth in SEQ ID NO:237.

In an exemplary embodiment, the single domain antibody or antigen binding fragment thereof comprises the amino acid sequence of the CDR3 set forth in SEQ ID NO:246 and the amino acid sequence of the FR2 set forth in SEQ ID NO:116 and optionally the amino acid sequence of the CDR1 set forth in SEQ ID NO:244 and/or the amino acid sequence of the CDR2 set forth in SEQ ID NO:245.

In an exemplary embodiment, the single domain antibody or antigen binding fragment thereof comprises the amino acid sequence of the CDR3 set forth in SEQ ID NO:303 and the amino acid sequence of the FR2 set forth in SEQ ID NO:116 and optionally the amino acid sequence of the CDR1 set forth in SEQ ID NO:301 and/or the amino acid sequence of the CDR2 set forth in SEQ ID NO:302.

In an exemplary embodiment, the single domain antibody or antigen binding fragment thereof comprises the amino acid sequence of the CDR3 set forth in SEQ ID NO:310 and the amino acid sequence of the FR2 set forth in SEQ ID NO:360 and optionally the amino acid sequence of the CDR1 set forth in SEQ ID NO:308 and/or the amino acid sequence of the CDR2 set forth in SEQ ID NO:309.

In an exemplary embodiment, the single domain antibody or antigen binding fragment thereof comprises the amino acid sequence of the CDR3 set forth in SEQ ID NO:317 and the amino acid sequence of the FR2 set forth in SEQ ID NO:354 and optionally the amino acid sequence of the CDR1 set forth in SEQ ID NO:315 and/or the amino acid sequence of the CDR2 set forth in SEQ ID NO:316.

In an exemplary embodiment, the single domain antibody or antigen binding fragment thereof comprises the amino acid sequence of the CDR3 set forth in SEQ ID NO:324 and the amino acid sequence of the FR2 set forth in SEQ ID NO:116 and optionally the amino acid sequence of the CDR1 set forth in SEQ ID NO:322 and/or the amino acid sequence of the CDR2 set forth in SEQ ID NO:323.

In an exemplary embodiment, the single domain antibody or antigen binding fragment thereof comprises the amino acid sequence of the CDR3 set forth in SEQ ID NO:331 and the amino acid sequence of the FR2 set forth in SEQ ID NO:355 and optionally the amino acid sequence of the CDR1 set forth in SEQ ID NO:329 and/or the amino acid sequence of the CDR2 set forth in SEQ ID NO:330.

In an exemplary embodiment, the single domain antibody or antigen binding fragment thereof comprises the amino acid sequence of the CDR3 set forth in SEQ ID NO:338 and the amino acid sequence of the FR2 set forth in SEQ ID NO:116 and optionally the amino acid sequence of the CDR1 set forth in SEQ ID NO:336 and/or the amino acid sequence of the CDR2 set forth in SEQ ID NO:337.

In an exemplary embodiment, the single domain antibody or antigen binding fragment thereof comprises the amino acid sequence of the CDR3 set forth in SEQ ID NO:345 and the amino acid sequence of the FR2 set forth in SEQ ID NO:356 and optionally the amino acid sequence of the CDR1 set forth in SEQ ID NO:343 and/or the amino acid sequence of the CDR2 set forth in SEQ ID NO:344.

In an exemplary embodiment, the single domain antibody or antigen binding fragment thereof comprises the amino acid sequence of the CDR3 set forth in SEQ ID NO:352 and the amino acid sequence of the FR2 set forth in SEQ ID NO:116 and optionally the amino acid sequence of the CDR1 set forth in SEQ ID NO:350 and/or the amino acid sequence of the CDR2 set forth in SEQ ID NO:351.

In an exemplary embodiment, the single domain antibody or antigen binding fragment thereof comprises the amino acid sequence of the CDR3 set forth in SEQ ID NO:396 and the amino acid sequence of the FR2 set forth in SEQ ID NO:116 and optionally the amino acid sequence of the CDR1 set forth in SEQ ID NO:394 and/or the amino acid sequence of the CDR2 set forth in SEQ ID NO:395.

In an exemplary embodiment, the single domain antibody or antigen binding fragment thereof comprises the amino acid sequence of the CDR3 set forth in SEQ ID NO:404 and the amino acid sequence of the FR2 set forth in SEQ ID NO:116 and optionally the amino acid sequence of the CDR1 set forth in SEQ ID NO:402 and/or the amino acid sequence of the CDR2 set forth in SEQ ID NO:403.

In an exemplary embodiment, the single domain antibody or antigen binding fragment thereof comprises the amino acid sequence of the CDR3 set forth in SEQ ID NO:411 and the amino acid sequence of the FR2 set forth in SEQ ID NO:116 and optionally the amino acid sequence of the CDR1 set forth in SEQ ID NO:409 and/or the amino acid sequence of the CDR2 set forth in SEQ ID NO:410.

In an exemplary embodiment, the single domain antibody or antigen binding fragment thereof comprises the amino acid sequence of the CDR3 set forth in SEQ ID NO:418 and the amino acid sequence of the FR2 set forth in SEQ ID NO:116 and optionally the amino acid sequence of the CDR1 set forth in SEQ ID NO:416 and/or the amino acid sequence of the CDR2 set forth in SEQ ID NO:416.

In an exemplary embodiment, the single domain antibody or antigen binding fragment thereof comprises the amino acid sequence of the CDR3 set forth in SEQ ID NO:425 and the amino acid sequence of the FR2 set forth in SEQ ID NO:116 and optionally the amino acid sequence of the CDR1 set forth in SEQ ID NO:423 and/or the amino acid sequence of the CDR2 set forth in SEQ ID NO:424.

In an exemplary embodiment, the single domain antibody or antigen binding fragment thereof comprises the amino acid sequence of the CDR3 set forth in SEQ ID NO:432 and the amino acid sequence of the FR2 set forth in SEQ ID NO:116 and optionally the amino acid sequence of the CDR1 set forth in SEQ ID NO:430 and/or the amino acid sequence of the CDR2 set forth in SEQ ID NO:431.

In an exemplary embodiment, the single domain antibody or antigen binding fragment thereof comprises the amino acid sequence of the CDR3 set forth in SEQ ID NO:439 and the amino acid sequence of the FR2 set forth in SEQ ID NO:116 and optionally the amino acid sequence of the CDR1 set forth in SEQ ID NO:437 and/or the amino acid sequence of the CDR2 set forth in SEQ ID NO:438.

In an exemplary embodiment, the single domain antibody or antigen binding fragment thereof comprises the amino acid sequence of the CDR3 set forth in SEQ ID NO:446 and the amino acid sequence of the FR2 set forth in SEQ ID NO:116 and optionally the amino acid sequence of the CDR1 set forth in SEQ ID NO:444 and/or the amino acid sequence of the CDR2 set forth in SEQ ID NO:445.

In an exemplary embodiment, the single domain antibody or antigen binding fragment thereof comprises the amino acid sequence of the CDR3 set forth in SEQ ID NO:453 and the amino acid sequence of the FR2 set forth in SEQ ID NO:360 and optionally the amino acid sequence of the CDR1 set forth in SEQ ID NO:451 and/or the amino acid sequence of the CDR2 set forth in SEQ ID NO:452.

In an exemplary embodiment, the single domain antibody or antigen binding fragment thereof comprises the amino acid sequence of the CDR3 set forth in SEQ ID NO:460 and the amino acid sequence of the FR2 set forth in SEQ ID NO:360 and optionally the amino acid sequence of the CDR1 set forth in SEQ ID NO:458 and/or the amino acid sequence of the CDR2 set forth in SEQ ID NO:459.

In an exemplary embodiment, the single domain antibody or antigen binding fragment thereof comprises the amino acid sequence of the CDR3 set forth in SEQ ID NO:467 and the amino acid sequence of the FR2 set forth in SEQ ID NO:116 and optionally the amino acid sequence of the CDR1 set forth in SEQ ID NO:465 and/or the amino acid sequence of the CDR2 set forth in SEQ ID NO:466.

In an exemplary embodiment, the single domain antibody or antigen binding fragment thereof comprises the amino acid sequence of the CDR3 set forth in SEQ ID NO:58 and the amino acid sequence of the FR2 set forth in SEQ ID NO:116 and optionally the amino acid sequence of the CDR1 set forth in SEQ ID NO:56 and/or the amino acid sequence of the CDR2 set forth in SEQ ID NO:57.

In an exemplary embodiment, the single domain antibody or antigen binding fragment thereof comprises the amino acid sequence of the CDR3 set forth in SEQ ID NO:61 and the amino acid sequence of the FR2 set forth in SEQ ID NO:116 and optionally the amino acid sequence of the CDR1 set forth in SEQ ID NO:59 and/or the amino acid sequence of the CDR2 set forth in SEQ ID NO:60.

In an exemplary embodiment, the single domain antibody or antigen binding fragment thereof comprises the amino acid sequence of the CDR3 set forth in SEQ ID NO:51 and the amino acid sequence of the FR2 set forth in SEQ ID NO:116 and optionally the amino acid sequence of the CDR1 set forth in SEQ ID NO:62 and/or the amino acid sequence of the CDR2 set forth in SEQ ID NO:50.

In an exemplary embodiment, the single domain antibody or antigen binding fragment thereof comprises the amino acid sequence of the CDR3 set forth in SEQ ID NO: 48 and the amino acid sequence of the FR2 set forth in SEQ ID NO:116 and optionally the amino acid sequence of the CDR1 set forth in SEQ ID NO:55 and/or the amino acid sequence of the CDR2 set forth in 47.

In an exemplary embodiment, the single domain antibody or antigen binding fragment thereof comprises the amino acid sequence of the CDR3 set forth in SEQ ID NO: 48 and the amino acid sequence of the FR2 set forth in SEQ ID NO:117 and optionally the amino acid sequence of the CDR1 set forth in SEQ ID NO:55 and/or the amino acid sequence of the CDR2 set forth in 47.

In an exemplary embodiment, the single domain antibody or antigen binding fragment thereof comprises the amino acid sequence of the CDR3 set forth in SEQ ID NO: 48 and the amino acid sequence of the FR2 set forth in SEQ ID NO:118 and optionally the amino acid sequence of the CDR1 set forth in SEQ ID NO:55 and/or the amino acid sequence of the CDR2 set forth in 47.

In an exemplary embodiment, the single domain antibody or antigen binding fragment thereof comprises the amino acid sequence of the CDR3 set forth in SEQ ID NO: 48 and the amino acid sequence of the FR2 set forth in SEQ ID NO:119 and optionally the amino acid sequence of the CDR1 set forth in SEQ ID NO:55 and/or the amino acid sequence of the CDR2 set forth in 47.

In an exemplary embodiment, the single domain antibody or antigen binding fragment thereof comprises the amino acid sequence of the CDR3 set forth in SEQ ID NO: 48 and the amino acid sequence of the FR2 set forth in SEQ ID NO:117 and optionally the amino acid sequence of the CDR1 set forth in SEQ ID NO:55 and/or the amino acid sequence of the CDR2 set forth in 124.

In an exemplary embodiment, the single domain antibody or antigen binding fragment thereof comprises the amino acid sequence of the CDR3 set forth in SEQ ID NO: 48 and the amino acid sequence of the FR2 set forth in SEQ ID NO:117 and optionally the amino acid sequence of the CDR1 set forth in SEQ ID NO:55 and/or the amino acid sequence of the CDR2 set forth in 123.

In an exemplary embodiment, the single domain antibody or antigen binding fragment thereof comprises the amino acid sequence of the CDR3 set forth in SEQ ID NO:51 and the amino acid sequence of the FR2 set forth in SEQ ID NO:116 and optionally the amino acid sequence of the CDR1 set forth in SEQ ID NO:62 and/or the amino acid sequence of the CDR2 set forth in SEQ ID NO:50.

In an exemplary embodiment, the single domain antibody or antigen binding fragment thereof comprises the amino acid sequence of the CDR3 set forth in SEQ ID NO:51 and the amino acid sequence of the FR2 set forth in SEQ ID NO:117 and optionally the amino acid sequence of the CDR1 set forth in SEQ ID NO:62 and/or the amino acid sequence of the CDR2 set forth in SEQ ID NO:50.

In an exemplary embodiment, the single domain antibody or antigen binding fragment thereof comprises the amino acid sequence of the CDR3 set forth in SEQ ID NO:51 and the amino acid sequence of the FR2 set forth in SEQ ID NO:118 and optionally the amino acid sequence of the CDR1 set forth in SEQ ID NO:62 and/or the amino acid sequence of the CDR2 set forth in SEQ ID NO:50.

In an exemplary embodiment, the single domain antibody or antigen binding fragment thereof comprises the amino acid sequence of the CDR3 set forth in SEQ ID NO:51 and the amino acid sequence of the FR2 set forth in SEQ ID NO:120 and optionally the amino acid sequence of the CDR1 set forth in SEQ ID NO:62 and/or the amino acid sequence of the CDR2 set forth in SEQ ID NO:50.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:170 and optionally the amino acid sequence of the CDR1 set forth in SEQ ID NO:4 and/or the amino acid sequence of the CDR2 set forth in SEQ ID NO:5.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:171 and optionally the amino acid sequence of the CDR1 set forth in SEQ ID NO:4 and/or the amino acid sequence of the CDR2 set forth in SEQ ID NO:130.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:170 and optionally the amino acid sequence of the CDR1 set forth in SEQ ID NO:4 and/or the amino acid sequence of the CDR2 set forth in SEQ ID NO:131.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:170 and optionally the amino acid sequence of the CDR1 set forth in SEQ ID NO:4 and/or the amino acid sequence of the CDR2 set forth in SEQ ID NO:133.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:171 and optionally the amino acid sequence of the CDR1 set forth in SEQ ID NO:4 and/or the amino acid sequence of the CDR2 set forth in SEQ ID NO:131.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:171 and optionally the amino acid sequence of the CDR1 set forth in SEQ ID NO:4 and/or the amino acid sequence of the CDR2 set forth in SEQ ID NO:132.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:171 and optionally the amino acid sequence of the CDR1 set forth in SEQ ID NO:4 and/or the amino acid sequence of the CDR2 set forth in SEQ ID NO:133.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:172 and optionally the amino acid sequence of the CDR1 set forth in SEQ ID NO:4 and/or the amino acid sequence of the CDR2 set forth in SEQ ID NO:133.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:171 and optionally the amino acid sequence of the CDR1 set forth in SEQ ID NO:4 and/or the amino acid sequence of the CDR2 set forth in SEQ ID NO:134.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:171 and optionally the amino acid sequence of the CDR1 set forth in SEQ ID NO:4 and/or the amino acid sequence of the CDR2 set forth in SEQ ID NO:135.

In an exemplary embodiment, the single domain antibody or antigen binding fragment thereof comprises the amino acid sequence of the CDR3 set forth in SEQ ID NO:6 and the amino acid sequence of the FR2 set forth in SEQ ID NO:174 and optionally the amino acid sequence of the CDR1 set forth in SEQ ID NO:127 and/or the amino acid sequence of the CDR2 set forth in SEQ ID NO:129.

In an exemplary embodiment, the single domain antibody or antigen binding fragment thereof comprises the amino acid sequence of the CDR3 set forth in SEQ ID NO:15 and the amino acid sequence of the FR2 set forth in SEQ ID NO:170 and optionally the amino acid sequence of the CDR1 set forth in SEQ ID NO:13 and/or the amino acid sequence of the CDR2 set forth in SEQ ID NO:14.

In an exemplary embodiment, the single domain antibody or antigen binding fragment thereof comprises the amino acid sequence of the CDR3 set forth in SEQ ID NO:15 and the amino acid sequence of the FR2 set forth in SEQ ID NO:170 and optionally the amino acid sequence of the CDR1 set forth in SEQ ID NO:13 and/or the amino acid sequence of the CDR2 set forth in SEQ ID NO:137.

In an exemplary embodiment, the single domain antibody or antigen binding fragment thereof comprises the amino acid sequence of the CDR3 set forth in SEQ ID NO:15 and the amino acid sequence of the FR2 set forth in SEQ ID NO:170 and optionally the amino acid sequence of the CDR1 set forth in SEQ ID NO:13 and/or the amino acid sequence of the CDR2 set forth in SEQ ID NO:138.

In an exemplary embodiment, the single domain antibody or antigen binding fragment thereof comprises the amino acid sequence of the CDR3 set forth in SEQ ID NO:217 and the amino acid sequence of the FR2 set forth in SEQ ID NO:170 and optionally the amino acid sequence of the CDR1 set forth in SEQ ID NO:215 and/or the amino acid sequence of the CDR2 set forth in SEQ ID NO:216.

In an exemplary embodiment, the single domain antibody or antigen binding fragment thereof comprises the amino acid sequence of the CDR3 set forth in SEQ ID NO:227 and the amino acid sequence of the FR2 set forth in SEQ ID NO:170 and optionally the amino acid sequence of the CDR1 set forth in SEQ ID NO:225 and/or the amino acid sequence of the CDR2 set forth in SEQ ID NO:226.

In an exemplary embodiment, the single domain antibody or antigen binding fragment thereof comprises the amino acid sequence of the CDR3 set forth in SEQ ID NO:235 and the amino acid sequence of the FR2 set forth in SEQ ID NO:170 and optionally the amino acid sequence of the CDR1 set forth in SEQ ID NO:233 and/or the amino acid sequence of the CDR2 set forth in SEQ ID NO:234.

In an exemplary embodiment, the single domain antibody or antigen binding fragment thereof comprises the amino acid sequence of the CDR3 set forth in SEQ ID NO:243 and the amino acid sequence of the FR2 set forth in SEQ ID NO:170 and optionally the amino acid sequence of the CDR1 set forth in SEQ ID NO:241 and/or the amino acid sequence of the CDR2 set forth in SEQ ID NO:242.

In an exemplary embodiment, the single domain antibody or antigen binding fragment thereof comprises the amino acid sequence of the CDR3 set forth in SEQ ID NO:300 and the amino acid sequence of the FR2 set forth in SEQ ID NO:170 and optionally the amino acid sequence of the CDR1 set forth in SEQ ID NO:298 and/or the amino acid sequence of the CDR2 set forth in SEQ ID NO:299.

In an exemplary embodiment, the single domain antibody or antigen binding fragment thereof comprises the amino acid sequence of the CDR3 set forth in SEQ ID NO:307 and the amino acid sequence of the FR2 set forth in SEQ ID NO:170 and optionally the amino acid sequence of the CDR1 set forth in SEQ ID NO:305 and/or the amino acid sequence of the CDR2 set forth in SEQ ID NO:306.

In an exemplary embodiment, the single domain antibody or antigen binding fragment thereof comprises the amino acid sequence of the CDR3 set forth in SEQ ID NO:314 and the amino acid sequence of the FR2 set forth in SEQ ID NO:357 and optionally the amino acid sequence of the CDR1 set forth in SEQ ID NO:312 and/or the amino acid sequence of the CDR2 set forth in SEQ ID NO:313.

In an exemplary embodiment, the single domain antibody or antigen binding fragment thereof comprises the amino acid sequence of the CDR3 set forth in SEQ ID NO:321 and the amino acid sequence of the FR2 set forth in SEQ ID NO:170 and optionally the amino acid sequence of the CDR1 set forth in SEQ ID NO:319 and/or the amino acid sequence of the CDR2 set forth in SEQ ID NO:320.

In an exemplary embodiment, the single domain antibody or antigen binding fragment thereof comprises the amino acid sequence of the CDR3 set forth in SEQ ID NO:328 and the amino acid sequence of the FR2 set forth in SEQ ID NO:358 and optionally the amino acid sequence of the CDR1 set forth in SEQ ID NO:326 and/or the amino acid sequence of the CDR2 set forth in SEQ ID NO:327.

In an exemplary embodiment, the single domain antibody or antigen binding fragment thereof comprises the amino acid sequence of the CDR3 set forth in SEQ ID NO:335 and the amino acid sequence of the FR2 set forth in SEQ ID NO:170 and optionally the amino acid sequence of the CDR1 set forth in SEQ ID NO:333 and/or the amino acid sequence of the CDR2 set forth in SEQ ID NO:334.

In an exemplary embodiment, the single domain antibody or antigen binding fragment thereof comprises the amino acid sequence of the CDR3 set forth in SEQ ID NO:342 and the amino acid sequence of the FR2 set forth in SEQ ID NO:359 and optionally the amino acid sequence of the CDR1 set forth in SEQ ID NO:340 and/or the amino acid sequence of the CDR2 set forth in SEQ ID NO:341.

In an exemplary embodiment, the single domain antibody or antigen binding fragment thereof comprises the amino acid sequence of the CDR3 set forth in SEQ ID NO:349 and the amino acid sequence of the FR2 set forth in SEQ ID NO:170 and optionally the amino acid sequence of the CDR1 set forth in SEQ ID NO:347 and/or the amino acid sequence of the CDR2 set forth in SEQ ID NO:348.

In an exemplary embodiment, the single domain antibody or antigen binding fragment thereof comprises the amino acid sequence of the CDR3 set forth in SEQ ID NO:393 and the amino acid sequence of the FR2 set forth in SEQ ID NO:170 and optionally the amino acid sequence of the CDR1 set forth in SEQ ID NO:391 and/or the amino acid sequence of the CDR2 set forth in SEQ ID NO:392.

In an exemplary embodiment, the single domain antibody or antigen binding fragment thereof comprises the amino acid sequence of the CDR3 set forth in SEQ ID NO:401 and the amino acid sequence of the FR2 set forth in SEQ ID NO:170 and optionally the amino acid sequence of the CDR1 set forth in SEQ ID NO:399 and/or the amino acid sequence of the CDR2 set forth in SEQ ID NO:400.

In an exemplary embodiment, the single domain antibody or antigen binding fragment thereof comprises the amino acid sequence of the CDR3 set forth in SEQ ID NO:408 and the amino acid sequence of the FR2 set forth in SEQ ID NO:170 and optionally the amino acid sequence of the CDR1 set forth in SEQ ID NO:406 and/or the amino acid sequence of the CDR2 set forth in SEQ ID NO:407.

In an exemplary embodiment, the single domain antibody or antigen binding fragment thereof comprises the amino acid sequence of the CDR3 set forth in SEQ ID NO:415 and the amino acid sequence of the FR2 set forth in SEQ ID NO:170 and optionally the amino acid sequence of the CDR1 set forth in SEQ ID NO:413 and/or the amino acid sequence of the CDR2 set forth in SEQ ID NO:414.

In an exemplary embodiment, the single domain antibody or antigen binding fragment thereof comprises the amino acid sequence of the CDR3 set forth in SEQ ID NO:422 and the amino acid sequence of the FR2 set forth in SEQ ID NO:170 and optionally the amino acid sequence of the CDR1 set forth in SEQ ID NO:420 and/or the amino acid sequence of the CDR2 set forth in SEQ ID NO:421.

In an exemplary embodiment, the single domain antibody or antigen binding fragment thereof comprises the amino acid sequence of the CDR3 set forth in SEQ ID NO:429 and the amino acid sequence of the FR2 set forth in SEQ ID NO:170 and optionally the amino acid sequence of the CDR1 set forth in SEQ ID NO:427 and/or the amino acid sequence of the CDR2 set forth in SEQ ID NO:428.

In an exemplary embodiment, the single domain antibody or antigen binding fragment thereof comprises the amino acid sequence of the CDR3 set forth in SEQ ID NO:436 and the amino acid sequence of the FR2 set forth in SEQ ID NO:170 and optionally the amino acid sequence of the CDR1 set forth in SEQ ID NO:434 and/or the amino acid sequence of the CDR2 set forth in SEQ ID NO:435.

In an exemplary embodiment, the single domain antibody or antigen binding fragment thereof comprises the amino acid sequence of the CDR3 set forth in SEQ ID NO:443 and the amino acid sequence of the FR2 set forth in SEQ ID NO:170 and optionally the amino acid sequence of the CDR1 set forth in SEQ ID NO:441 and/or the amino acid sequence of the CDR2 set forth in SEQ ID NO:442.

In an exemplary embodiment, the single domain antibody or antigen binding fragment thereof comprises the amino acid sequence of the CDR3 set forth in SEQ ID NO:450 and the amino acid sequence of the FR2 set forth in SEQ ID NO:170 and optionally the amino acid sequence of the CDR1 set forth in SEQ ID NO:448 and/or the amino acid sequence of the CDR2 set forth in SEQ ID NO:449.

In an exemplary embodiment, the single domain antibody or antigen binding fragment thereof comprises the amino acid sequence of the CDR3 set forth in SEQ ID NO:457 and the amino acid sequence of the FR2 set forth in SEQ ID NO:170 and optionally the amino acid sequence of the CDR1 set forth in SEQ ID NO:455 and/or the amino acid sequence of the CDR2 set forth in SEQ ID NO:456.

In an exemplary embodiment, the single domain antibody or antigen binding fragment thereof comprises the amino acid sequence of the CDR3 set forth in SEQ ID NO:464 and the amino acid sequence of the FR2 set forth in SEQ ID NO:170 and optionally the amino acid sequence of the CDR1 set forth in SEQ ID NO:462 and/or the amino acid sequence of the CDR2 set forth in SEQ ID NO:463.

In an exemplary embodiment, the single domain antibody or antigen binding fragment thereof comprises the amino acid sequence of the CDR3 set forth in SEQ ID NO:15 and the amino acid sequence of the FR2 set forth in SEQ ID NO:171 and optionally the amino acid sequence of the CDR1 set forth in SEQ ID NO:13 and/or the amino acid sequence of the CDR2 set forth in SEQ ID NO:136.

In an exemplary embodiment, the single domain antibody or antigen binding fragment thereof comprises the amino acid sequence of the CDR3 set forth in SEQ ID NO:15 and the amino acid sequence of the FR2 set forth in SEQ ID NO:171 and optionally the amino acid sequence of the CDR1 set forth in SEQ ID NO:13 and/or the amino acid sequence of the CDR2 set forth in SEQ ID NO:137.

In an exemplary embodiment, the single domain antibody or antigen binding fragment thereof comprises the amino acid sequence of the CDR3 set forth in SEQ ID NO:15 and the amino acid sequence of the FR2 set forth in SEQ ID NO:171 and optionally the amino acid sequence of the CDR1 set forth in SEQ ID NO:13 and/or the amino acid sequence of the CDR2 set forth in SEQ ID NO:138.

In an exemplary embodiment, the single domain antibody or antigen binding fragment thereof comprises the amino acid sequence of the CDR3 set forth in SEQ ID NO:15 and the amino acid sequence of the FR2 set forth in SEQ ID NO:171 and optionally the amino acid sequence of the CDR1 set forth in SEQ ID NO:13 and/or the amino acid sequence of the CDR2 set forth in SEQ ID NO:139.

In an exemplary embodiment, the single domain antibody or antigen binding fragment thereof comprises the amino acid sequence of the CDR3 set forth in SEQ ID NO:15 and the amino acid sequence of the FR2 set forth in SEQ ID NO:173 and optionally the amino acid sequence of the CDR1 set forth in SEQ ID NO:13 and/or the amino acid sequence of the CDR2 set forth in SEQ ID NO:137.

In some embodiments single domain antibody or antigen binding fragment thereof of the present disclosure comprises the three CDRs of the single domain antibody disclosed herein.

In an exemplary embodiment, the single domain antibody or antigen binding fragment thereof comprises a CDR1 having the amino acid sequence set forth in SEQ ID NO:1, a heavy chain complementarity determining region 2 (CDRH2) having the amino acid sequence set forth in SEQ ID NO:2 and a heavy chain complementarity determining region 3 (CDRH3) having the amino acid sequence set forth in SEQ ID NO:3.

In an exemplary embodiment, the single domain antibody or antigen binding fragment thereof comprises a CDRH1 having the amino acid sequence set forth in SEQ ID NO:4, a CDRH2 having the amino acid sequence set forth in SEQ ID NO:5 and a CDRH3 having the amino acid sequence set forth in SEQ ID NO:6.

In another exemplary embodiment, the single domain antibody or antigen binding fragment thereof comprises a CDRH1 having the amino acid sequence set forth in SEQ ID NO:7, a CDRH2 having the amino acid sequence set forth in SEQ ID NO:8 and a CDRH3 having the amino acid sequence set forth in SEQ ID NO:9.

In another exemplary embodiment, the single domain antibody or antigen binding fragment thereof comprises a CDRH1 having the amino acid sequence set forth in SEQ ID NO:10, a CDRH2 having the amino acid sequence set forth in SEQ ID NO:11 and a CDRH3 having the amino acid sequence set forth in SEQ ID NO:12.

In another exemplary embodiment, the single domain antibody or antigen binding fragment thereof comprises a CDRH1 having the amino acid sequence set forth in SEQ ID NO:13, a CDRH2 having the amino acid sequence set forth in SEQ ID NO:14 and a CDRH3 having the amino acid sequence set forth in SEQ ID NO:15.

In another exemplary embodiment, the single domain antibody or antigen binding fragment thereof comprises a CDRH1 having the amino acid sequence set forth in SEQ ID NO:16, a CDRH2 having the amino acid sequence set forth in SEQ ID NO:17 and a CDRH3 having the amino acid sequence set forth in SEQ ID NO:18.

In another exemplary embodiment, the single domain antibody or antigen binding fragment thereof comprises a CDRH1 having the amino acid sequence set forth in SEQ ID NO:19, a CDRH2 having the amino acid sequence set forth in SEQ ID NO:20 and a CDRH3 having the amino acid sequence set forth in SEQ ID NO:21.

In other exemplary embodiments, the single domain antibody comprises a CDRH1 having the amino acid sequence set forth in SEQ ID NO:46, a CDRH2 having the amino acid sequence set forth in SEQ ID NO:47 and a CDRH3 having the amino acid sequence set forth in SEQ ID NO:48.

In other exemplary embodiments, the single domain antibody comprises a CDRH1 having the amino acid sequence set forth in SEQ ID NO:55, a CDRH2 having the amino acid sequence set forth in SEQ ID NO:47 and a CDRH3 having the amino acid sequence set forth in SEQ ID NO:48.

In other exemplary embodiments, the single domain antibody comprises a CDRH1 having the amino acid sequence set forth in SEQ ID NO:49, a CDRH2 having the amino acid sequence set forth in SEQ ID NO:50 and a CDRH3 having the amino acid sequence set forth in SEQ ID NO:51.

In other exemplary embodiments, the single domain antibody comprises a CDRH1 having the amino acid sequence set forth in SEQ ID NO:62, a CDRH2 having the amino acid sequence set forth in SEQ ID NO:50 and a CDRH3 having the amino acid sequence set forth in SEQ ID NO:51.

In other exemplary embodiments, the single domain antibody comprises a CDRH1 having the amino acid sequence set forth in SEQ ID NO:52, a CDRH2 having the amino acid sequence set forth in SEQ ID NO:53 and a CDRH3 having the amino acid sequence set forth in SEQ ID NO:54.

In other exemplary embodiments, the single domain antibody comprises a CDRH1 having the amino acid sequence set forth in SEQ ID NO:56, a CDRH2 having the amino acid sequence set forth in SEQ ID NO:57 and a CDRH3 having the amino acid sequence set forth in SEQ ID NO:58.

In other exemplary embodiments, the single domain antibody comprises a CDRH1 having the amino acid sequence set forth in SEQ ID NO:59, a CDRH2 having the amino acid sequence set forth in SEQ ID NO:60 and a CDRH3 having the amino acid sequence set forth in SEQ ID NO:61.

In other exemplary embodiments, the single domain antibody comprises a CDRH1 having the amino acid sequence set forth in SEQ ID NO:63, a CDRH2 having the amino acid sequence set forth in SEQ ID NO:64 and a CDRH3 having the amino acid sequence set forth in SEQ ID NO:65.

In other exemplary embodiments, the single domain antibody comprises a CDRH1 having the amino acid sequence set forth in SEQ ID NO:66, a CDRH2 having the amino acid sequence set forth in SEQ ID NO:67 and a CDRH3 having the amino acid sequence set forth in SEQ ID NO:68.

In another exemplary embodiment, the single domain antibody or antigen binding fragment thereof comprises the amino acid sequence of the CDR1 set forth in SEQ ID NO:55, the amino acid sequence of the CDR2 set forth in SEQ ID NO:123 and the amino acid sequence of the CDR3 set forth in SEQ ID NO: 48.

In another exemplary embodiment, the single domain antibody or antigen binding fragment thereof comprises the amino acid sequence of the CDR1 set forth in SEQ ID NO:55, the amino acid sequence of the CDR2 set forth in SEQ ID NO:124 and the amino acid sequence of the CDR3 set forth in SEQ ID NO: 48.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:130 and the amino acid sequence of the CDR3 set forth in SEQ ID NO:6.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:131 and the amino acid sequence of the CDR3 set forth in SEQ ID NO:6.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:132 and the amino acid sequence of the CDR3 set forth in SEQ ID NO:6.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:133 and the amino acid sequence of the CDR3 set forth in SEQ ID NO:6.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:134 and the amino acid sequence of the CDR3 set forth in SEQ ID NO:6.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:135 and the amino acid sequence of the CDR3 set forth in SEQ ID NO:6.

In an exemplary embodiment, the single domain antibody or antigen binding fragment thereof comprises the amino acid sequence of the CDR1 set forth in SEQ ID NO:127, the amino acid sequence of the CDR2 set forth in SEQ ID NO:129 and the amino acid sequence of the CDR3 set forth in SEQ ID NO:6.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:14 and the amino acid sequence of the CDR3 set forth in SEQ ID NO:15.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:136 and the amino acid sequence of the CDR3 set forth in SEQ ID NO:15.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:137 and the amino acid sequence of the CDR3 set forth in SEQ ID NO:15.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:138 and the amino acid sequence of the CDR3 set forth in SEQ ID NO:15.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:139 and the amino acid sequence of the CDR3 set forth in SEQ ID NO:15.

In an exemplary embodiment, the single domain antibody or antigen binding fragment thereof comprises the amino acid sequence of the CDR1 set forth in SEQ ID NO:215, the amino acid sequence of the CDR2 set forth in SEQ ID NO:216 and the amino acid sequence of the CDR3 set forth in SEQ ID NO:217.

In an exemplary embodiment, the single domain antibody or antigen binding fragment thereof comprises the amino acid sequence of the CDR1 set forth in SEQ ID NO:218, the amino acid sequence of the CDR2 set forth in SEQ ID NO:219 and the amino acid sequence of the CDR3 set forth in SEQ ID NO:220.

In an exemplary embodiment, the single domain antibody or antigen binding fragment thereof comprises the amino acid sequence of the CDR1 set forth in SEQ ID NO:225, the amino acid sequence of the CDR2 set forth in SEQ ID NO:226 and the amino acid sequence of the CDR3 set forth in SEQ ID NO:227.

In an exemplary embodiment, the single domain antibody or antigen binding fragment thereof comprises the amino acid sequence of the CDR1 set forth in SEQ ID NO:228, the amino acid sequence of the CDR2 set forth in SEQ ID NO:229 and the amino acid sequence of the CDR3 set forth in SEQ ID NO:230.

In an exemplary embodiment, the single domain antibody or antigen binding fragment thereof comprises the amino acid sequence of the CDR1 set forth in SEQ ID NO:233, the amino acid sequence of the CDR2 set forth in SEQ ID NO:234 and the amino acid sequence of the CDR3 set forth in SEQ ID NO:235.

In an exemplary embodiment, the single domain antibody or antigen binding fragment thereof comprises the amino acid sequence of the CDR1 set forth in SEQ ID NO:236, the amino acid sequence of the CDR2 set forth in SEQ ID NO:237 and the amino acid sequence of the CDR3 set forth in SEQ ID NO:238.

In an exemplary embodiment, the single domain antibody or antigen binding fragment thereof comprises the amino acid sequence of the CDR1 set forth in SEQ ID NO:241, the amino acid sequence of the CDR2 set forth in SEQ ID NO:242 and the amino acid sequence of the CDR3 set forth in SEQ ID NO:243.

In an exemplary embodiment, the single domain antibody or antigen binding fragment thereof comprises the amino acid sequence of the CDR1 set forth in SEQ ID NO:244, the amino acid sequence of the CDR2 set forth in SEQ ID NO:245 and the amino acid sequence of the CDR3 set forth in SEQ ID NO:246.

In an exemplary embodiment, the single domain antibody or antigen binding fragment thereof comprises the amino acid sequence of the CDR1 set forth in SEQ ID NO:298, the amino acid sequence of the CDR2 set forth in SEQ ID NO:299 and the amino acid sequence of the CDR3 set forth in SEQ ID NO:300.

In an exemplary embodiment, the single domain antibody or antigen binding fragment thereof comprises the amino acid sequence of the CDR1 set forth in SEQ ID NO:301, the amino acid sequence of the CDR2 set forth in SEQ ID NO:302 and the amino acid sequence of the CDR3 set forth in SEQ ID NO:303.

In an exemplary embodiment, the single domain antibody or antigen binding fragment thereof comprises the amino acid sequence of the CDR1 set forth in SEQ ID NO:305, the amino acid sequence of the CDR2 set forth in SEQ ID NO:306 and the amino acid sequence of the CDR3 set forth in SEQ ID NO:307

In an exemplary embodiment, the single domain antibody or antigen binding fragment thereof comprises the amino acid sequence of the CDR1 set forth in SEQ ID NO:308, the amino acid sequence of the CDR2 set forth in SEQ ID NO:309 and the amino acid sequence of the CDR3 set forth in SEQ ID NO:310.

In an exemplary embodiment, the single domain antibody or antigen binding fragment thereof comprises the amino acid sequence of the CDR1 set forth in SEQ ID NO:312, the amino acid sequence of the CDR2 set forth in SEQ ID NO:313 and the amino acid sequence of the CDR3 set forth in SEQ ID NO:314.

In an exemplary embodiment, the single domain antibody or antigen binding fragment thereof comprises the amino acid sequence of the CDR1 set forth in SEQ ID NO:315, the amino acid sequence of the CDR2 set forth in SEQ ID NO:316 and the amino acid sequence of the CDR3 set forth in SEQ ID NO:317.

In an exemplary embodiment, the single domain antibody or antigen binding fragment thereof comprises the amino acid sequence of the CDR1 set forth in SEQ ID NO:319, the amino acid sequence of the CDR2 set forth in SEQ ID NO:320 and the amino acid sequence of the CDR3 set forth in SEQ ID NO:321.

In an exemplary embodiment, the single domain antibody or antigen binding fragment thereof comprises the amino acid sequence of the CDR1 set forth in SEQ ID NO:322, the amino acid sequence of the CDR2 set forth in SEQ ID NO:323 and the amino acid sequence of the CDR3 set forth in SEQ ID NO:324.

In an exemplary embodiment, the single domain antibody or antigen binding fragment thereof comprises the amino acid sequence of the CDR1 set forth in SEQ ID NO:326, the amino acid sequence of the CDR2 set forth in SEQ ID NO:327 and the amino acid sequence of the CDR3 set forth in SEQ ID NO:328.

In an exemplary embodiment, the single domain antibody or antigen binding fragment thereof comprises the amino acid sequence of the CDR1 set forth in SEQ ID NO:329, the amino acid sequence of the CDR2 set forth in SEQ ID NO:330 and the amino acid sequence of the CDR3 set forth in SEQ ID NO:331.

In an exemplary embodiment, the single domain antibody or antigen binding fragment thereof comprises the amino acid sequence of the CDR1 set forth in SEQ ID NO:333, the amino acid sequence of the CDR2 set forth in SEQ ID NO:334 and the amino acid sequence of the CDR3 set forth in SEQ ID NO:335.

In an exemplary embodiment, the single domain antibody or antigen binding fragment thereof comprises the amino acid sequence of the CDR1 set forth in SEQ ID NO:336, the amino acid sequence of the CDR2 set forth in SEQ ID NO:337 and the amino acid sequence of the CDR3 set forth in SEQ ID NO:338.

In an exemplary embodiment, the single domain antibody or antigen binding fragment thereof comprises the amino acid sequence of the CDR1 set forth in SEQ ID NO:340, the amino acid sequence of the CDR2 set forth in SEQ ID NO:341 and the amino acid sequence of the CDR3 set forth in SEQ ID NO:342.

In an exemplary embodiment, the single domain antibody or antigen binding fragment thereof comprises the amino acid sequence of the CDR1 set forth in SEQ ID NO:343, the amino acid sequence of the CDR2 set forth in SEQ ID NO:344 and the amino acid sequence of the CDR3 set forth in SEQ ID NO:345.

In an exemplary embodiment, the single domain antibody or antigen binding fragment thereof comprises the amino acid sequence of the CDR1 set forth in SEQ ID NO:347, the amino acid sequence of the CDR2 set forth in SEQ ID NO:348 and the amino acid sequence of the CDR3 set forth in SEQ ID NO:349

In an exemplary embodiment, the single domain antibody or antigen binding fragment thereof comprises the amino acid sequence of the CDR1 set forth in SEQ ID NO:350, the amino acid sequence of the CDR2 set forth in SEQ ID NO:351 and the amino acid sequence of the CDR3 set forth in SEQ ID NO:352.

In an exemplary embodiment, the single domain antibody or antigen binding fragment thereof comprises the amino acid sequence of the CDR1 set forth in SEQ ID NO:391, the amino acid sequence of the CDR2 set forth in SEQ ID NO:392 and the amino acid sequence of the CDR3 set forth in SEQ ID NO:393.

In an exemplary embodiment, the single domain antibody or antigen binding fragment thereof comprises the amino acid sequence of the CDR1 set forth in SEQ ID NO:394, the amino acid sequence of the CDR2 set forth in SEQ ID NO:395 and the amino acid sequence of the CDR3 set forth in SEQ ID NO:396.

In an exemplary embodiment, the single domain antibody or antigen binding fragment thereof comprises the amino acid sequence of the CDR1 set forth in SEQ ID NO:399, the amino acid sequence of the CDR2 set forth in SEQ ID NO:400 and the amino acid sequence of the CDR3 set forth in SEQ ID NO:401.

In an exemplary embodiment, the single domain antibody or antigen binding fragment thereof comprises the amino acid sequence of the CDR1 set forth in SEQ ID NO:402, the amino acid sequence of the CDR2 set forth in SEQ ID NO:403 and the amino acid sequence of the CDR3 set forth in SEQ ID NO:404.

In an exemplary embodiment, the single domain antibody or antigen binding fragment thereof comprises the amino acid sequence of the CDR1 set forth in SEQ ID NO:406, the amino acid sequence of the CDR2 set forth in SEQ ID NO:407 and the amino acid sequence of the CDR3 set forth in SEQ ID NO:408.

In an exemplary embodiment, the single domain antibody or antigen binding fragment thereof comprises the amino acid sequence of the CDR1 set forth in SEQ ID NO:409, the amino acid sequence of the CDR2 set forth in SEQ ID NO:410 and the amino acid sequence of the CDR3 set forth in SEQ ID NO:411.

In an exemplary embodiment, the single domain antibody or antigen binding fragment thereof comprises the amino acid sequence of the CDR1 set forth in SEQ ID NO:413, the amino acid sequence of the CDR2 set forth in SEQ ID NO:414 and the amino acid sequence of the CDR3 set forth in SEQ ID NO:415.

In an exemplary embodiment, the single domain antibody or antigen binding fragment thereof comprises the amino acid sequence of the CDR1 set forth in SEQ ID NO:416, the amino acid sequence of the CDR2 set forth in SEQ ID NO:417 and the amino acid sequence of the CDR3 set forth in SEQ ID NO:418.

In an exemplary embodiment, the single domain antibody or antigen binding fragment thereof comprises the amino acid sequence of the CDR1 set forth in SEQ ID NO:420, the amino acid sequence of the CDR2 set forth in SEQ ID NO:421 and the amino acid sequence of the CDR3 set forth in SEQ ID NO:422.

In an exemplary embodiment, the single domain antibody or antigen binding fragment thereof comprises the amino acid sequence of the CDR1 set forth in SEQ ID NO:423, the amino acid sequence of the CDR2 set forth in SEQ ID NO:424 and the amino acid sequence of the CDR3 set forth in SEQ ID NO:425.

In an exemplary embodiment, the single domain antibody or antigen binding fragment thereof comprises the amino acid sequence of the CDR1 set forth in SEQ ID NO:427, the amino acid sequence of the CDR2 set forth in SEQ ID NO:428 and the amino acid sequence of the CDR3 set forth in SEQ ID NO:429.

In an exemplary embodiment, the single domain antibody or antigen binding fragment thereof comprises the amino acid sequence of the CDR1 set forth in SEQ ID NO:430, the amino acid sequence of the CDR2 set forth in SEQ ID NO:431 and the amino acid sequence of the CDR3 set forth in SEQ ID NO:432.

In an exemplary embodiment, the single domain antibody or antigen binding fragment thereof comprises the amino acid sequence of the CDR1 set forth in SEQ ID NO:434, the amino acid sequence of the CDR2 set forth in SEQ ID NO:435 and the amino acid sequence of the CDR3 set forth in SEQ ID NO:436.

In an exemplary embodiment, the single domain antibody or antigen binding fragment thereof comprises the amino acid sequence of the CDR1 set forth in SEQ ID NO:437, the amino acid sequence of the CDR2 set forth in SEQ ID NO:438 and the amino acid sequence of the CDR3 set forth in SEQ ID NO:439.

In an exemplary embodiment, the single domain antibody or antigen binding fragment thereof comprises the amino acid sequence of the CDR1 set forth in SEQ ID NO:441, the amino acid sequence of the CDR2 set forth in SEQ ID NO:442 and the amino acid sequence of the CDR3 set forth in SEQ ID NO:443.

In an exemplary embodiment, the single domain antibody or antigen binding fragment thereof comprises the amino acid sequence of the CDR1 set forth in SEQ ID NO:444, the amino acid sequence of the CDR2 set forth in SEQ ID NO:445 and the amino acid sequence of the CDR3 set forth in SEQ ID NO:446.

In an exemplary embodiment, the single domain antibody or antigen binding fragment thereof comprises the amino acid sequence of the CDR1 set forth in SEQ ID NO:448, the amino acid sequence of the CDR2 set forth in SEQ ID NO:449 and the amino acid sequence of the CDR3 set forth in SEQ ID NO:450.

In an exemplary embodiment, the single domain antibody or antigen binding fragment thereof comprises the amino acid sequence of the CDR1 set forth in SEQ ID NO:451, the amino acid sequence of the CDR2 set forth in SEQ ID NO:452 and the amino acid sequence of the CDR3 set forth in SEQ ID NO:453.

In an exemplary embodiment, the single domain antibody or antigen binding fragment thereof comprises the amino acid sequence of the CDR1 set forth in SEQ ID NO:455, the amino acid sequence of the CDR2 set forth in SEQ ID NO:456 and the amino acid sequence of the CDR3 set forth in SEQ ID NO:457.

In an exemplary embodiment, the single domain antibody or antigen binding fragment thereof comprises the amino acid sequence of the CDR1 set forth in SEQ ID NO:458, the amino acid sequence of the CDR2 set forth in SEQ ID NO:459 and the amino acid sequence of the CDR3 set forth in SEQ ID NO:460.

In an exemplary embodiment, the single domain antibody or antigen binding fragment thereof comprises the amino acid sequence of the CDR1 set forth in SEQ ID NO:462, the amino acid sequence of the CDR2 set forth in SEQ ID NO:463 and the amino acid sequence of the CDR3 set forth in SEQ ID NO:464.

In an exemplary embodiment, the single domain antibody or antigen binding fragment thereof comprises the amino acid sequence of the CDR1 set forth in SEQ ID NO:465, the amino acid sequence of the CDR2 set forth in SEQ ID NO:466 and the amino acid sequence of the CDR3 set forth in SEQ ID NO:467.

In an exemplary embodiment, the single domain antibody or antigen binding fragment thereof comprises the amino acid sequence of the CDR1 set forth in SEQ ID NO:485, the amino acid sequence of the CDR2 set forth in SEQ ID NO:486 and the amino acid sequence of the CDR3 set forth in SEQ ID NO:487.

In an exemplary embodiment, the single domain antibody or antigen binding fragment thereof comprises the amino acid sequence of the CDR1 set forth in SEQ ID NO:488, the amino acid sequence of the CDR2 set forth in SEQ ID NO:489 and the amino acid sequence of the CDR3 set forth in SEQ ID NO:490.

In an exemplary embodiment, the single domain antibody or antigen binding fragment thereof comprises the amino acid sequence of the CDR1 set forth in SEQ ID NO:485, the amino acid sequence of the CDR2 set forth in SEQ ID NO:492 and the amino acid sequence of the CDR3 set forth in SEQ ID NO:487.

In an exemplary embodiment, the single domain antibody or antigen binding fragment thereof comprises the amino acid sequence of the CDR1 set forth in SEQ ID NO:485, the amino acid sequence of the CDR2 set forth in SEQ ID NO:495 and the amino acid sequence of the CDR3 set forth in SEQ ID NO:487.

In an exemplary embodiment, the single domain antibody or antigen binding fragment thereof comprises the amino acid sequence of the CDR1 set forth in SEQ ID NO:485, the amino acid sequence of the CDR2 set forth in SEQ ID NO:498 and the amino acid sequence of the CDR3 set forth in SEQ ID NO:499.

In an exemplary embodiment, the single domain antibody or antigen binding fragment thereof comprises the amino acid sequence of the CDR1 set forth in SEQ ID NO:500, the amino acid sequence of the CDR2 set forth in SEQ ID NO:489 and the amino acid sequence of the CDR3 set forth in SEQ ID NO:501.

In an exemplary embodiment, the single domain antibody or antigen binding fragment thereof comprises the amino acid sequence of the CDR1 set forth in SEQ ID NO:485, the amino acid sequence of the CDR2 set forth in SEQ ID NO:486 and the amino acid sequence of the CDR3 set forth in SEQ ID NO:499.

In some embodiments single domain antibody or antigen binding fragment thereof of the present disclosure comprises the CDR1, CDR2, CDR3 and the FR2 amino acid sequence of the single domain antibody disclosed herein.

In another exemplary embodiment, the single domain antibody or antigen binding fragment thereof comprises the amino acid sequence of the CDR1 set forth in SEQ ID NO:52, the amino acid sequence of the CDR2 set forth in SEQ ID NO:53, the amino acid sequence of the CDR3 set forth in SEQ ID NO:54 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:55, the amino acid sequence of the CDR2 set forth in SEQ ID NO:47, the amino acid sequence of the CDR3 set forth in SEQ ID NO:48 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:56, the amino acid sequence of the CDR2 set forth in SEQ ID NO:57, the amino acid sequence of the CDR3 set forth in SEQ ID NO:58 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:59, the amino acid sequence of the CDR2 set forth in SEQ ID NO:60, the amino acid sequence of the CDR3 set forth in SEQ ID NO:61 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:62, the amino acid sequence of the CDR2 set forth in SEQ ID NO:50, the amino acid sequence of the CDR3 set forth in SEQ ID NO:51 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:55, the amino acid sequence of the CDR2 set forth in SEQ ID NO:47, the amino acid sequence of the CDR3 set forth in SEQ ID NO: 48 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:218, the amino acid sequence of the CDR2 set forth in SEQ ID NO:219, the amino acid sequence of the CDR3 set forth in SEQ ID NO: 220 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:218, the amino acid sequence of the CDR2 set forth in SEQ ID NO:219, the amino acid sequence of the CDR3 set forth in SEQ ID NO: 220 and the amino acid sequence of the FR2 set forth in SEQ ID NO:117.

In another exemplary embodiment, the single domain antibody or antigen binding fragment thereof comprises the amino acid sequence of the CDR1 set forth in SEQ ID NO:228, the amino acid sequence of the CDR2 set forth in SEQ ID NO:229, the amino acid sequence of the CDR3 set forth in SEQ ID NO: 230 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:236, the amino acid sequence of the CDR2 set forth in SEQ ID NO:237, the amino acid sequence of the CDR3 set forth in SEQ ID NO: 238 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:244, the amino acid sequence of the CDR2 set forth in SEQ ID NO:245, the amino acid sequence of the CDR3 set forth in SEQ ID NO: 246 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:301, the amino acid sequence of the CDR2 set forth in SEQ ID NO:302, the amino acid sequence of the CDR3 set forth in SEQ ID NO: 303 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:308, the amino acid sequence of the CDR2 set forth in SEQ ID NO:309, the amino acid sequence of the CDR3 set forth in SEQ ID NO: 310 and the amino acid sequence of the FR2 set forth in SEQ ID NO:360.

In another exemplary embodiment, the single domain antibody or antigen binding fragment thereof comprises the amino acid sequence of the CDR1 set forth in SEQ ID NO:315, the amino acid sequence of the CDR2 set forth in SEQ ID NO:316, the amino acid sequence of the CDR3 set forth in SEQ ID NO: 317 and the amino acid sequence of the FR2 set forth in SEQ ID NO:354.

In another exemplary embodiment, the single domain antibody or antigen binding fragment thereof comprises the amino acid sequence of the CDR1 set forth in SEQ ID NO:322, the amino acid sequence of the CDR2 set forth in SEQ ID NO:323, the amino acid sequence of the CDR3 set forth in SEQ ID NO: 324 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:329, the amino acid sequence of the CDR2 set forth in SEQ ID NO:330, the amino acid sequence of the CDR3 set forth in SEQ ID NO: 331 and the amino acid sequence of the FR2 set forth in SEQ ID NO:355.

In another exemplary embodiment, the single domain antibody or antigen binding fragment thereof comprises the amino acid sequence of the CDR1 set forth in SEQ ID NO:336, the amino acid sequence of the CDR2 set forth in SEQ ID NO:337, the amino acid sequence of the CDR3 set forth in SEQ ID NO: 338 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:343, the amino acid sequence of the CDR2 set forth in SEQ ID NO:344, the amino acid sequence of the CDR3 set forth in SEQ ID NO: 345 and the amino acid sequence of the FR2 set forth in SEQ ID NO:356.

In another exemplary embodiment, the single domain antibody or antigen binding fragment thereof comprises the amino acid sequence of the CDR1 set forth in SEQ ID NO:350, the amino acid sequence of the CDR2 set forth in SEQ ID NO:351, the amino acid sequence of the CDR3 set forth in SEQ ID NO: 352 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:394, the amino acid sequence of the CDR2 set forth in SEQ ID NO:395, the amino acid sequence of the CDR3 set forth in SEQ ID NO: 396 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:402, the amino acid sequence of the CDR2 set forth in SEQ ID NO:403, the amino acid sequence of the CDR3 set forth in SEQ ID NO: 404 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:409, the amino acid sequence of the CDR2 set forth in SEQ ID NO:410, the amino acid sequence of the CDR3 set forth in SEQ ID NO: 411 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:416, the amino acid sequence of the CDR2 set forth in SEQ ID NO:417, the amino acid sequence of the CDR3 set forth in SEQ ID NO: 418 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:423, the amino acid sequence of the CDR2 set forth in SEQ ID NO:424, the amino acid sequence of the CDR3 set forth in SEQ ID NO: 425 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:430, the amino acid sequence of the CDR2 set forth in SEQ ID NO:431, the amino acid sequence of the CDR3 set forth in SEQ ID NO: 432 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:437, the amino acid sequence of the CDR2 set forth in SEQ ID NO:438, the amino acid sequence of the CDR3 set forth in SEQ ID NO: 439 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:444, the amino acid sequence of the CDR2 set forth in SEQ ID NO:445, the amino acid sequence of the CDR3 set forth in SEQ ID NO: 446 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:451, the amino acid sequence of the CDR2 set forth in SEQ ID NO:452, the amino acid sequence of the CDR3 set forth in SEQ ID NO: 453 and the amino acid sequence of the FR2 set forth in SEQ ID NO:360.

In another exemplary embodiment, the single domain antibody or antigen binding fragment thereof comprises the amino acid sequence of the CDR1 set forth in SEQ ID NO:458, the amino acid sequence of the CDR2 set forth in SEQ ID NO:459, the amino acid sequence of the CDR3 set forth in SEQ ID NO: 460 and the amino acid sequence of the FR2 set forth in SEQ ID NO:360.

In another exemplary embodiment, the single domain antibody or antigen binding fragment thereof comprises the amino acid sequence of the CDR1 set forth in SEQ ID NO:465, the amino acid sequence of the CDR2 set forth in SEQ ID NO:466, the amino acid sequence of the CDR3 set forth in SEQ ID NO: 467 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:488, the amino acid sequence of the CDR2 set forth in SEQ ID NO:489, the amino acid sequence of the CDR3 set forth in SEQ ID NO: 490 and the amino acid sequence of the FR2 set forth in SEQ ID NO:505.

In another exemplary embodiment, the single domain antibody or antigen binding fragment thereof comprises the amino acid sequence of the CDR1 set forth in SEQ ID NO:500, the amino acid sequence of the CDR2 set forth in SEQ ID NO:489, the amino acid sequence of the CDR3 set forth in SEQ ID NO: 501 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:55, the amino acid sequence of the CDR2 set forth in SEQ ID NO:47, the amino acid sequence of the CDR3 set forth in SEQ ID NO: 48 and the amino acid sequence of the FR2 set forth in SEQ ID NO:117.

In another exemplary embodiment, the single domain antibody or antigen binding fragment thereof comprises the amino acid sequence of the CDR1 set forth in SEQ ID NO:55, the amino acid sequence of the CDR2 set forth in SEQ ID NO: 47, the amino acid sequence of the CDR3 set forth in SEQ ID NO: 48 and the amino acid sequence of the FR2 set forth in SEQ ID NO:118.

In another exemplary embodiment, the single domain antibody or antigen binding fragment thereof comprises the amino acid sequence of the CDR1 set forth in SEQ ID NO:55, the amino acid sequence of the CDR2 set forth in SEQ ID NO:47, the amino acid sequence of the CDR3 set forth in SEQ ID NO: 48 and the amino acid sequence of the FR2 set forth in SEQ ID NO:119.

In another exemplary embodiment, the single domain antibody or antigen binding fragment thereof comprises the amino acid sequence of the CDR1 set forth in SEQ ID NO:55, the amino acid sequence of the CDR2 set forth in SEQ ID NO:124, the amino acid sequence of the CDR3 set forth in SEQ ID NO: 48 and the amino acid sequence of the FR2 set forth in SEQ ID NO:117.

In another exemplary embodiment, the single domain antibody or antigen binding fragment thereof comprises the amino acid sequence of the CDR1 set forth in SEQ ID NO:55, the amino acid sequence of the CDR2 set forth in SEQ ID NO:123, the amino acid sequence of the CDR3 set forth in SEQ ID NO: 48 and the amino acid sequence of the FR2 set forth in SEQ ID NO:117.

In another exemplary embodiment, the single domain antibody or antigen binding fragment thereof comprises the amino acid sequence of the CDR1 set forth in SEQ ID NO:62, the amino acid sequence of the CDR2 set forth in SEQ ID NO:50, the amino acid sequence of the CDR3 set forth in SEQ ID NO:51 and the amino acid sequence of the FR2 set forth in SEQ ID NO:116.

In another exemplary embodiment, the single domain antibody or antigen binding fragment thereof comprises the amino acid sequence of the CDR1 set forth in SEQ ID NO:62, the amino acid sequence of the CDR2 set forth in SEQ ID NO:50, the amino acid sequence of the CDR3 set forth in SEQ ID NO:51 and the amino acid sequence of the FR2 set forth in SEQ ID NO:117.

In another exemplary embodiment, the single domain antibody or antigen binding fragment thereof comprises the amino acid sequence of the CDR1 set forth in SEQ ID NO:62, the amino acid sequence of the CDR2 set forth in SEQ ID NO:50, amino acid sequence of the CDR3 set forth in SEQ ID NO:51 and the amino acid sequence of the FR2 set forth in SEQ ID NO:118.

In another exemplary embodiment, the single domain antibody or antigen binding fragment thereof comprises the amino acid sequence of the CDR1 set forth in SEQ ID NO:62, the amino acid sequence of the CDR2 set forth in SEQ ID NO:50, amino acid sequence of the CDR3 set forth in SEQ ID NO:51 and the amino acid sequence of the FR2 set forth in SEQ ID NO:120.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:5, the amino acid sequence of the CDR3 set forth in SEQ ID NO:6 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:130, the amino acid sequence of the CDR3 set forth in SEQ ID NO:6 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:131, the amino acid sequence of the CDR3 set forth in SEQ ID NO:6 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:133, the amino acid sequence of the CDR3 set forth in SEQ ID NO:6 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:131, the amino acid sequence of the CDR3 set forth in SEQ ID NO:6 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:132, the amino acid sequence of the CDR3 set forth in SEQ ID NO:6 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:133, the amino acid sequence of the CDR3 set forth in SEQ ID NO:6 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:133, the amino acid sequence of the CDR3 set forth in SEQ ID NO:6 and the amino acid sequence of the FR2 set forth in SEQ ID NO:172.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:134, the amino acid sequence of the CDR3 set forth in SEQ ID NO:6 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:4, the amino acid sequence of the CDR2 set forth in SEQ ID NO:135, the amino acid sequence of the CDR3 set forth in SEQ ID NO:6 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:127, the amino acid sequence of the CDR2 set forth in SEQ ID NO:129, the amino acid sequence of the CDR3 set forth in SEQ ID NO:6 and the amino acid sequence of the FR2 set forth in SEQ ID NO:174.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:14, the amino acid sequence of the CDR3 set forth in SEQ ID NO:15 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:137, the amino acid sequence of the CDR3 set forth in SEQ ID NO:15 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:138, the amino acid sequence of the CDR3 set forth in SEQ ID NO:15 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:215, the amino acid sequence of the CDR2 set forth in SEQ ID NO:216, the amino acid sequence of the CDR3 set forth in SEQ ID NO:217 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:225, the amino acid sequence of the CDR2 set forth in SEQ ID NO:226, the amino acid sequence of the CDR3 set forth in SEQ ID NO:227 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:233, the amino acid sequence of the CDR2 set forth in SEQ ID NO:234, the amino acid sequence of the CDR3 set forth in SEQ ID NO:235 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:241, the amino acid sequence of the CDR2 set forth in SEQ ID NO:242, the amino acid sequence of the CDR3 set forth in SEQ ID NO:243 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:298, the amino acid sequence of the CDR2 set forth in SEQ ID NO:299, the amino acid sequence of the CDR3 set forth in SEQ ID NO:300 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:305, the amino acid sequence of the CDR2 set forth in SEQ ID NO:306, the amino acid sequence of the CDR3 set forth in SEQ ID NO:307 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:312, the amino acid sequence of the CDR2 set forth in SEQ ID NO:313, the amino acid sequence of the CDR3 set forth in SEQ ID NO:314 and the amino acid sequence of the FR2 set forth in SEQ ID NO:357.

In an exemplary embodiment, the single domain antibody or antigen binding fragment thereof comprises the amino acid sequence of the CDR1 set forth in SEQ ID NO:319, the amino acid sequence of the CDR2 set forth in SEQ ID NO:320, the amino acid sequence of the CDR3 set forth in SEQ ID NO:321 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:326, the amino acid sequence of the CDR2 set forth in SEQ ID NO:327, the amino acid sequence of the CDR3 set forth in SEQ ID NO:328 and the amino acid sequence of the FR2 set forth in SEQ ID NO:358.

In an exemplary embodiment, the single domain antibody or antigen binding fragment thereof comprises the amino acid sequence of the CDR1 set forth in SEQ ID NO:333, the amino acid sequence of the CDR2 set forth in SEQ ID NO:334, the amino acid sequence of the CDR3 set forth in SEQ ID NO:335 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:340, the amino acid sequence of the CDR2 set forth in SEQ ID NO:341, the amino acid sequence of the CDR3 set forth in SEQ ID NO:342 and the amino acid sequence of the FR2 set forth in SEQ ID NO:359.

In an exemplary embodiment, the single domain antibody or antigen binding fragment thereof comprises the amino acid sequence of the CDR1 set forth in SEQ ID NO:347, the amino acid sequence of the CDR2 set forth in SEQ ID NO:348, the amino acid sequence of the CDR3 set forth in SEQ ID NO:349 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:391, the amino acid sequence of the CDR2 set forth in SEQ ID NO:392, the amino acid sequence of the CDR3 set forth in SEQ ID NO:393 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:399, the amino acid sequence of the CDR2 set forth in SEQ ID NO:400, the amino acid sequence of the CDR3 set forth in SEQ ID NO:401 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:406, the amino acid sequence of the CDR2 set forth in SEQ ID NO:407, the amino acid sequence of the CDR3 set forth in SEQ ID NO:408 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:413, the amino acid sequence of the CDR2 set forth in SEQ ID NO:414, the amino acid sequence of the CDR3 set forth in SEQ ID NO:415 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:420, the amino acid sequence of the CDR2 set forth in SEQ ID NO:421, the amino acid sequence of the CDR3 set forth in SEQ ID NO:422 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:427, the amino acid sequence of the CDR2 set forth in SEQ ID NO:428, the amino acid sequence of the CDR3 set forth in SEQ ID NO:429 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:434, the amino acid sequence of the CDR2 set forth in SEQ ID NO:435, the amino acid sequence of the CDR3 set forth in SEQ ID NO:436 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:441, the amino acid sequence of the CDR2 set forth in SEQ ID NO:442, the amino acid sequence of the CDR3 set forth in SEQ ID NO:443 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:448, the amino acid sequence of the CDR2 set forth in SEQ ID NO:449, the amino acid sequence of the CDR3 set forth in SEQ ID NO:450 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:455, the amino acid sequence of the CDR2 set forth in SEQ ID NO:456, the amino acid sequence of the CDR3 set forth in SEQ ID NO:457 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:462, the amino acid sequence of the CDR2 set forth in SEQ ID NO:463, the amino acid sequence of the CDR3 set forth in SEQ ID NO:464 and the amino acid sequence of the FR2 set forth in SEQ ID NO:170.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:136, the amino acid sequence of the CDR3 set forth in SEQ ID NO:15 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:13 and/or the amino acid sequence of the CDR2 set forth in SEQ ID NO:137, the amino acid sequence of the CDR3 set forth in SEQ ID NO:15 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:138, the amino acid sequence of the CDR3 set forth in SEQ ID NO:15 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:139, the amino acid sequence of the CDR3 set forth in SEQ ID NO:15 and the amino acid sequence of the FR2 set forth in SEQ ID NO:171.

In an exemplary embodiment, the single domain antibody or antigen binding fragment thereof comprises the amino acid sequence of the CDR1 set forth in SEQ ID NO:13, the amino acid sequence of the CDR2 set forth in SEQ ID NO:137, the amino acid sequence of the CDR3 set forth in SEQ ID NO:15 and the amino acid sequence of the FR2 set forth in SEQ ID NO:173.

In another exemplary embodiment, the single domain antibody or antigen binding fragment thereof comprises the variable region of the single domain antibody disclosed herein.

In further exemplary embodiments, the single domain antibody comprises a variable region having an amino acid sequence as set forth in SEQ ID NO:22.

In other exemplary embodiments, the single domain antibody comprises a variable region having an amino acid sequence as set forth in SEQ ID NO:23.

In further exemplary embodiments, the single domain antibody comprises a variable region having an amino acid sequence as set forth in SEQ ID NO:24.

In additional exemplary embodiments, the single domain antibody comprises a variable region having an amino acid sequence as set forth in SEQ ID NO:25.

In other exemplary embodiments, the single domain antibody comprises a variable region having an amino acid sequence as set forth in SEQ ID NO:26.

In further exemplary embodiments, the single domain antibody comprises a variable region having an amino acid sequence as set forth in SEQ ID NO:27.

In yet further exemplary embodiments, the single domain antibody comprises a variable region having an amino acid sequence as set forth in SEQ ID NO:28.

In yet further exemplary embodiments, the single domain antibody comprises a variable region having an amino acid sequence as set forth in SEQ ID NO:221.

In yet further exemplary embodiments, the single domain antibody comprises a variable region having an amino acid sequence as set forth in SEQ ID NO:222.

In yet further exemplary embodiments, the single domain antibody comprises a variable region having an amino acid sequence as set forth in SEQ ID NO:223.

In yet further exemplary embodiments, the single domain antibody comprises a variable region having an amino acid sequence as set forth in SEQ ID NO:224.

In yet further exemplary embodiments, the single domain antibody comprises a variable region having an amino acid sequence as set forth in SEQ ID NO:231.

In yet further exemplary embodiments, the single domain antibody comprises a variable region having an amino acid sequence as set forth in SEQ ID NO:232.

In yet further exemplary embodiments, the single domain antibody comprises a variable region having an amino acid sequence as set forth in SEQ ID NO:239.

In yet further exemplary embodiments, the single domain antibody comprises a variable region having an amino acid sequence as set forth in SEQ ID NO:240.

In yet further exemplary embodiments, the single domain antibody comprises a variable region having an amino acid sequence as set forth in SEQ ID NO:247.

In yet further exemplary embodiments, the single domain antibody comprises a variable region having an amino acid sequence as set forth in SEQ ID NO:304.

In yet further exemplary embodiments, the single domain antibody comprises a variable region having an amino acid sequence as set forth in SEQ ID NO:311.

In yet further exemplary embodiments, the single domain antibody comprises a variable region having an amino acid sequence as set forth in SEQ ID NO:318.

In yet further exemplary embodiments, the single domain antibody comprises a variable region having an amino acid sequence as set forth in SEQ ID NO:325.

In yet further exemplary embodiments, the single domain antibody comprises a variable region having an amino acid sequence as set forth in SEQ ID NO:332.

In yet further exemplary embodiments, the single domain antibody comprises a variable region having an amino acid sequence as set forth in SEQ ID NO:339.

In yet further exemplary embodiments, the single domain antibody comprises a variable region having an amino acid sequence as set forth in SEQ ID NO:346.

In yet further exemplary embodiments, the single domain antibody comprises a variable region having an amino acid sequence as set forth in SEQ ID NO:353.

In some embodiments, the single domain antibody may comprise a sequence made from the combination of the CDRH1, CDRH2 and CDRH3 and heavy chain variable region backbone set forth in Table 23 or Table 24.

Accordingly, in some embodiments, the single domain antibody may comprise a sequence as set forth in Table 1A.

| CDRH1; CDRH2; CDRH3 | Exemplary heavy chain variable region backbone |
|---|---|
| SEQ ID NO: 52; SEQ ID NO: 53; SEQ | SEQ ID NOs: 248, 249, 250, 251, 252, 253, 254, |
| ID NO: 54 or | 255, 256, 257, 258, 259, 260, 261, 262, 263, 264, |
| SEQ ID NO: 218; SEQ ID NO: 219; | 265, 266, 267, 268, 269, 270, or 271 |
| SEQ ID NO: 220 | |
| SEQ ID NO: 52; SEQ ID NO: 53; SEQ | SEQ ID NOs: 272, 273, 274, 275, 276, 277, 278, |
| ID NO: 54 or | 279, 280, 281, 282, 283, 284, 285, 286, 287, 288, |
| SEQ ID NO: 218; SEQ ID NO: 219; | 289, 290, 291, 292, 293 or 294. |
| SEQ ID NO: 220 | SEQ ID NOs 474, 475 or 476 |
| SEQ ID NO 55; SEQ ID NO: 47; SEQ | SEQ ID NOs: 248, 249, 250, 251, 252, 253, 254, |
| ID NO: 48 | 255, 256, 257, 258, 259, 260, 261, 262, 263, 264, |
| | 265, 266, 267, 268, 269, 270, or 271 |
| SEQ ID NO 55; SEQ ID NO: 47; SEQ | SEQ ID NOs: 272, 273, 274, 275, 276, 277, 278, |
| ID NO: 48 | 279, 280, 281, 282, 283, 284, 285, 286, 287, 288, |
| | 289, 290, 291, 292, 293 or 294. |
| SEQ ID NO: 56; SEQ ID NO: 57; SEQ | SEQ ID NOs: 248, 249, 250, 251, 252, 253, 254, |
| ID NO: 58 | 255, 256, 257, 258, 259, 260, 261, 262, 263, 264, |
| | 265, 266, 267, 268, 269, 270, or 271 |
| SEQ ID NO: 56; SEQ ID NO: 57; SEQ | SEQ ID NOs: 272, 273, 274, 275, 276, 277, 278, |
| ID NO: 58 | 279, 280, 281, 282, 283, 284, 285, 286, 287, 288, |
| | 289, 290, 291, 292, 293 or 294. |
| SEQ ID NO: 59; SEQ ID NO: 60; SEQ | SEQ ID NOs: 248, 249, 250, 251, 252, 253, 254, |
| ID NO: 61 or | 255, 256, 257, 258, 259, 260, 261, 262, 263, 264, |
| SEQ ID NO: 301; SEQ ID NO: 302; | 265, 266, 267, 268, 269, 270, or 271 |
| SEQ ID NO: 303 | |
| SEQ ID NO: 59; SEQ ID NO: 60; SEQ | SEQ ID NOs: 272, 273, 274, 275, 276, 277, 278, |
| ID NO: 61 or | 279, 280, 281, 282, 283, 284, 285, 286, 287, 288, |
| SEQ ID NO: 301; SEQ ID NO: 302; | 289, 290, 291, 292, 293 or 294. |
| SEQ ID NO: 303 | |
| SEQ ID NO: 62; SEQ ID NO: 50; SEQ | SEQ ID NOs: 248, 249, 250, 251, 252, 253, 254, |
| ID NO: 51 | 255, 256, 257, 258, 259, 260, 261, 262, 263, 264, |
| | 265, 266, 267, 268, 269, 270, or 271 |
| SEQ ID NO: 62; SEQ ID NO: 50; SEQ | SEQ ID NOs: 272, 273, 274, 275, 276, 277, 278, |
| ID NO: 51 | 279, 280, 281, 282, 283, 284, 285, 286, 287, 288, |
| | 289, 290, 291, 292, 293 or 294. |
| SEQ ID NO: 228; SEQ ID NO: 229; | SEQ ID NOs: 248, 249, 250, 251, 252, 253, 254, |
| SEQ ID NO: 230 | 255, 256, 257, 258, 259, 260, 261, 262, 263, 264, |
| | 265, 266, 267, 268, 269, 270, or 271 |
| SEQ ID NO: 228; SEQ ID NO: 229; | SEQ ID NOs: 272, 273, 274, 275, 276, 277, 278, |
| SEQ ID NO: 230 | 279, 280, 281, 282, 283, 284, 285, 286, 287, 288, |
| | 289, 290, 291, 292, 293 or 294. |
| SEQ ID NO: 236; SEQ ID NO: 237; | SEQ ID NOs: 248, 249, 250, 251, 252, 253, 254, |
| SEQ ID NO: 238 | 255, 256, 257, 258, 259, 260, 261, 262, 263, 264, |
| | 265, 266, 267, 268, 269, 270, or 271 |
| SEQ ID NO: 236; SEQ ID NO: 237; | SEQ ID NOs: 272, 273, 274, 275, 276, 277, 278, |
| SEQ ID NO: 238 | 279, 280, 281, 282, 283, 284, 285, 286, 287, 288, |
| | 289, 290, 291, 292, 293 or 294. |
| SEQ ID NO: 244; SEQ ID NO: 245; | SEQ ID NOs: 248, 249, 250, 251, 252, 253, 254, |
| SEQ ID NO: 246 | 255, 256, 257, 258, 259, 260, 261, 262, 263, 264, |
| | 265, 266, 267, 268, 269, 270, or 271 |
| SEQ ID NO: 244; SEQ ID NO: 245; | SEQ ID NOs: 272, 273, 274, 275, 276, 277, 278, |
| SEQ ID NO: 246 | 279, 280, 281, 282, 283, 284, 285, 286, 287, 288, |
| | 289, 290, 291, 292, 293 or 294. |
| SEQ ID NO: 308; SEQ ID NO: 309; | SEQ ID NOs: 248, 249, 250, 251, 252, 253, 254, |
| SEQ ID NO: 310 | 255, 256, 257, 258, 259, 260, 261, 262, 263, 264, |
| | 265, 266, 267, 268, 269, 270, or 271 |
| SEQ ID NO: 308; SEQ ID NO: 309; | SEQ ID NOs: 272, 273, 274, 275, 276, 277, 278, |
| SEQ ID NO: 310 | 279, 280, 281, 282, 283, 284, 285, 286, 287, 288, |
| | 289, 290, 291, 292, 293 or 294. |
| SEQ ID NO: 315; SEQ ID NO: 316; | SEQ ID NOs: 248, 249, 250, 251, 252, 253, 254, |
| SEQ ID NO: 317 | 255, 256, 257, 258, 259, 260, 261, 262, 263, 264, |
| | 265, 266, 267, 268, 269, 270, or 271 |
| SEQ ID NO: 315; SEQ ID NO: 316; | SEQ ID NOs: 272, 273, 274, 275, 276, 277, 278, |
| SEQ ID NO: 317 | 279, 280, 281, 282, 283, 284, 285, 286, 287, 288, |
| | 289, 290, 291, 292, 293 or 294. |
| SEQ ID NO: 322; SEQ ID NO: 323; | SEQ ID NOs: 248, 249, 250, 251, 252, 253, 254, |
| SEQ ID NO: 324 | 255, 256, 257, 258, 259, 260, 261, 262, 263, 264, |
| | 265, 266, 267, 268, 269, 270, or 271 |
| SEQ ID NO: 322; SEQ ID NO: 323; | SEQ ID NOs: 272, 273, 274, 275, 276, 277, 278, |
| SEQ ID NO: 324 | 279, 280, 281, 282, 283, 284, 285, 286, 287, 288, |
| | 289, 290, 291, 292, 293 or 294. |
| SEQ ID NO: 329; SEQ ID NO: 330; | SEQ ID NOs: 248, 249, 250, 251, 252, 253, 254, |
| SEQ ID NO: 331 | 255, 256, 257, 258, 259, 260, 261, 262, 263, 264, |
| | 265, 266, 267, 268, 269, 270, or 271 |
| SEQ ID NO: 329; SEQ ID NO: 330; | SEQ ID NOs: 272, 273, 274, 275, 276, 277, 278, |
| SEQ ID NO: 331 | 279, 280, 281, 282, 283, 284, 285, 286, 287, 288, |
| | 289, 290, 291, 292, 293 or 294. |
| SEQ ID NO: 336; SEQ ID NO: 337; | SEQ ID NOs: 248, 249, 250, 251, 252, 253, 254, |
| SEQ ID NO: 338 | 255, 256, 257, 258, 259, 260, 261, 262, 263, 264, |
| | 265, 266, 267, 268, 269, 270, or 271 |
| SEQ ID NO: 336; SEQ ID NO: 337; | SEQ ID NOs: 272, 273, 274, 275, 276, 277, 278, |
| SEQ ID NO: 338 | 279, 280, 281, 282, 283, 284, 285, 286, 287, 288, |
| | 289, 290, 291, 292, 293 or 294. |
| SEQ ID NO: 343; SEQ ID NO: 344; | SEQ ID NOs: 248, 249, 250, 251, 252, 253, 254, |
| SEQ ID NO: 345 | 255, 256, 257, 258, 259, 260, 261, 262, 263, 264, |
| | 265, 266, 267, 268, 269, 270, or 271 |
| SEQ ID NO: 343; SEQ ID NO: 344; | SEQ ID NOs: 272, 273, 274, 275, 276, 277, 278, |
| SEQ ID NO: 345 | 279, 280, 281, 282, 283, 284, 285, 286, 287, 288, |
| | 289, 290, 291, 292, 293 or 294. |
| SEQ ID NO: 350; SEQ ID NO: 351; | SEQ ID NOs: 248, 249, 250, 251, 252, 253, 254, |
| SEQ ID NO: 352 | 255, 256, 257, 258, 259, 260, 261, 262, 263, 264, |
| | 265, 266, 267, 268, 269, 270, or 271 |
| SEQ ID NO: 350; SEQ ID NO: 351; | SEQ ID NOs: 272, 273, 274, 275, 276, 277, 278, |
| SEQ ID NO: 352 | 279, 280, 281, 282, 283, 284, 285, 286, 287, 288, |
| | 289, 290, 291, 292, 293 or 294. |
| SEQ ID NO: 394; SEQ ID NO: 395; | SEQ ID NOs: 248, 249, 250, 251, 252, 253, 254, |
| SEQ ID NO: 396 | 255, 256, 257, 258, 259, 260, 261, 262, 263, 264, |
| | 265, 266, 267, 268, 269, 270, or 271 |
| SEQ ID NO: 394; SEQ ID NO: 395; | SEQ ID NOs: 272, 273, 274, 275, 276, 277, 278, |
| SEQ ID NO: 396 | 279, 280, 281, 282, 283, 284, 285, 286, 287, 288, |
| | 289, 290, 291, 292, 293 or 294. |
| SEQ ID NO: 402; SEQ ID NO: 403; | SEQ ID NOs: 248, 249, 250, 251, 252, 253, 254, |
| SEQ ID NO: 404 | 255, 256, 257, 258, 259, 260, 261, 262, 263, 264, |
| | 265, 266, 267, 268, 269, 270, or 271 |
| SEQ ID NO: 402; SEQ ID NO: 403; | SEQ ID NOs: 272, 273, 274, 275, 276, 277, 278, |
| SEQ ID NO: 404 | 279, 280, 281, 282, 283, 284, 285, 286, 287, 288, |
| | 289, 290, 291, 292, 293 or 294. |
| SEQ ID NO: 409; SEQ ID NO: 410; | SEQ ID NOs: 248, 249, 250, 251, 252, 253, 254, |
| SEQ ID NO: 411 | 255, 256, 257, 258, 259, 260, 261, 262, 263, 264, |
| | 265, 266, 267, 268, 269, 270, or 271 |
| SEQ ID NO: 409; SEQ ID NO: 410; | SEQ ID NOs: 272, 273, 274, 275, 276, 277, 278, |
| SEQ ID NO: 411 | 279, 280, 281, 282, 283, 284, 285, 286, 287, 288, |
| | 289, 290, 291, 292, 293 or 294. |
| SEQ ID NO: 416; SEQ ID NO: 417; | SEQ ID NOs: 248, 249, 250, 251, 252, 253, 254, |
| SEQ ID NO: 418 | 255, 256, 257, 258, 259, 260, 261, 262, 263, 264, |
| | 265, 266, 267, 268, 269, 270, or 271 |
| SEQ ID NO: 416; SEQ ID NO: 417; | SEQ ID NOs: 272, 273, 274, 275, 276, 277, 278, |
| SEQ ID NO: 418 | 279, 280, 281, 282, 283, 284, 285, 286, 287, 288, |
| | 289, 290, 291, 292, 293 or 294. |
| SEQ ID NO: 423; SEQ ID NO: 424; | SEQ ID NOs: 248, 249, 250, 251, 252, 253, 254, |
| SEQ ID NO: 425 | 255, 256, 257, 258, 259, 260, 261, 262, 263, 264, |
| | 265, 266, 267, 268, 269, 270, or 271 |
| SEQ ID NO: 423; SEQ ID NO: 424; | SEQ ID NOs: 272, 273, 274, 275, 276, 277, 278, |
| SEQ ID NO: 425 | 279, 280, 281, 282, 283, 284, 285, 286, 287, 288, |
| | 289, 290, 291, 292, 293 or 294. |
| SEQ ID NO: 430; SEQ ID NO: 431; | SEQ ID NOs: 248, 249, 250, 251, 252, 253, 254, |
| SEQ ID NO: 432 | 255, 256, 257, 258, 259, 260, 261, 262, 263, 264, |
| | 265, 266, 267, 268, 269, 270, or 271 |
| SEQ ID NO: 430; SEQ ID NO: 431; | SEQ ID NOs: 272, 273, 274, 275, 276, 277, 278, |
| SEQ ID NO: 432 | 279, 280, 281, 282, 283, 284, 285, 286, 287, 288, |
| | 289, 290, 291, 292, 293 or 294. |
| SEQ ID NO: 437; SEQ ID NO: 438; | SEQ ID NOs: 248, 249, 250, 251, 252, 253, 254, |
| SEQ ID NO: 439 | 255, 256, 257, 258, 259, 260, 261, 262, 263, 264, |
| | 265, 266, 267, 268, 269, 270, or 271 |
| SEQ ID NO: 437; SEQ ID NO: 438; | SEQ ID NOs: 272, 273, 274, 275, 276, 277, 278, |
| SEQ ID NO: 439 | 279, 280, 281, 282, 283, 284, 285, 286, 287, 288, |
| | 289, 290, 291, 292, 293 or 294. |
| SEQ ID NO: 444; SEQ ID NO: 445; | SEQ ID NOs: 248, 249, 250, 251, 252, 253, 254, |
| SEQ ID NO: 446 | 255, 256, 257, 258, 259, 260, 261, 262, 263, 264, |
| | 265, 266, 267, 268, 269, 270, or 271 |
| SEQ ID NO: 444; SEQ ID NO: 445; | SEQ ID NOs: 272, 273, 274, 275, 276, 277, 278, |
| SEQ ID NO: 446 | 279, 280, 281, 282, 283, 284, 285, 286, 287, 288, |
| | 289, 290, 291, 292, 293 or 294. |
| SEQ ID NO: 451; SEQ ID NO: 452; | SEQ ID NOs: 248, 249, 250, 251, 252, 253, 254, |
| SEQ ID NO: 453 | 255, 256, 257, 258, 259, 260, 261, 262, 263, 264, |
| | 265, 266, 267, 268, 269, 270, or 271 |
| SEQ ID NO: 451; SEQ ID NO: 452; | SEQ ID NOs: 272, 273, 274, 275, 276, 277, 278, |
| SEQ ID NO: 453 | 279, 280, 281, 282, 283, 284, 285, 286, 287, 288, |
| | 289, 290, 291, 292, 293 or 294. |
| CDRH1; CDRH2; CDRH3 | Exemplary heavy chain variable region backbone |
| SEQ ID NO: 458; SEQ ID NO: 459; | SEQ ID NOs: 248, 249, 250, 251, 252, 253, 254, |
| SEQ ID NO: 460 | 255, 256, 257, 258, 259, 260, 261, 262, 263, 264, |
| | 265, 266, 267, 268, 269, 270, or 271 |
| SEQ ID NO: 458; SEQ ID NO: 459; | SEQ ID NOs: 272, 273, 274, 275, 276, 277, 278, |
| SEQ ID NO: 460 | 279, 280, 281, 282, 283, 284, 285, 286, 287, 288, |
| | 289, 290, 291, 292, 293 or 294. |
| SEQ ID NO: 465; SEQ ID NO: 466; | SEQ ID NOs: 248, 249, 250, 251, 252, 253, 254, |
| SEQ ID NO: 467 | 255, 256, 257, 258, 259, 260, 261, 262, 263, 264, |
| | 265, 266, 267, 268, 269, 270, or 271 |
| SEQ ID NO: 465; SEQ ID NO: 466; | SEQ ID NOs: 272, 273, 274, 275, 276, 277, 278, |
| SEQ ID NO: 467 | 279, 280, 281, 282, 283, 284, 285, 286, 287, 288, |
| | 289, 290, 291, 292, 293 or 294. |
| SEQ ID NO: 488; SEQ ID NO: 489; | SEQ ID NOs: 248, 249, 250, 251, 252, 253, 254, |
| SEQ ID NO: 490 | 255, 256, 257, 258, 259, 260, 261, 262, 263, 264, |
| | 265, 266, 267, 268, 269, 270, or 271 |
| SEQ ID NO: 488; SEQ ID NO: 489; | SEQ ID NOs: 272, 273, 274, 275, 276, 277, 278, |
| SEQ ID NO: 490 | 279, 280, 281, 282, 283, 284, 285, 286, 287, 288, |
| | 289, 290, 291, 292, 293 or 294. |
| SEQ ID NO: 500; SEQ ID NO: 489; | SEQ ID NOs: 248, 249, 250, 251, 252, 253, 254, |
| SEQ ID NO: 501 | 255, 256, 257, 258, 259, 260, 261, 262, 263, 264, |
| | 265, 266, 267, 268, 269, 270, or 271 |
| SEQ ID NO: 500; SEQ ID NO: 489; | SEQ ID NOs: 272, 273, 274, 275, 276, 277, 278, |
| SEQ ID NO: 501 | 279, 280, 281, 282, 283, 284, 285, 286, 287, 288, |
| | 289, 290, 291, 292, 293 or 294. |

It is to be understood herein that the position of the residues referred in Table 23 and Table 24 takes into account the insertion of the complete amino acid sequence of each of the IMGT CDR1, CDR2 and CDR3 into the humanized heavy chain variable region backbone and with reference to the position of amino acid residues attributed to SEQ ID NO: 145 of Table 1C. As such, upon inserting the complete amino acid sequence of each of the IMGT CDR1, CDR2 and CDR3, the IMGT framework 1 will generally cover position 1 to 25, the IMGT framework 2, will generally cover position 34 to 50, the IMGT framework 3 will generally cover position 58 to 95 and the IMGT framework 4 will generally cover position 110-120. Since the length of the IMGT CDRs may vary slightly, the positioning of the frameworks may vary accordingly. However, a person skilled in the art will be able to determine the position number of a given variable region by alignment with SEQ ID NO:145 of Table 1C.

In additional exemplary embodiments, the single domain antibody comprises an amino acid sequence as set forth in SEQ ID NO:29.

In additional exemplary embodiments, the single domain antibody comprises an amino acid sequence as set forth in SEQ ID NO:71.

In additional exemplary embodiments, the single domain antibody comprises an amino acid sequence as set forth in SEQ ID NO:72.

In additional exemplary embodiments, the single domain antibody comprises an amino acid sequence as set forth in SEQ ID NO:73.

In additional exemplary embodiments, the single domain antibody comprises an amino acid sequence as set forth in SEQ ID NO:74.

In additional exemplary embodiments, the single domain antibody comprises an amino acid sequence as set forth in SEQ ID NO:75.

In additional exemplary embodiments, the single domain antibody comprises an amino acid sequence as set forth in SEQ ID NO:76.

In additional exemplary embodiments, the single domain antibody comprises an amino acid sequence as set forth in SEQ ID NO:77.

In additional exemplary embodiments, the single domain antibody comprises an amino acid sequence as set forth in SEQ ID NO:78.

In additional exemplary embodiments, the single domain antibody comprises an amino acid sequence as set forth in SEQ ID NO:79.

In additional exemplary embodiments, the single domain antibody comprises an amino acid sequence as set forth in SEQ ID NO:80.

In additional exemplary embodiments, the single domain antibody comprises an amino acid sequence as set forth in SEQ ID NO:81.

In additional exemplary embodiments, the single domain antibody comprises an amino acid sequence as set forth in SEQ ID NO:82.

In additional exemplary embodiments, the single domain antibody comprises an amino acid sequence as set forth in SEQ ID NO:83.

In additional exemplary embodiments, the single domain antibody comprises an amino acid sequence as set forth in SEQ ID NO:84.

In additional exemplary embodiments, the single domain antibody comprises an amino acid sequence as set forth in SEQ ID NO:85.

In additional exemplary embodiments, the single domain antibody comprises an amino acid sequence as set forth in SEQ ID NO:86.

In additional exemplary embodiments, the single domain antibody comprises an amino acid sequence as set forth in SEQ ID NO:87.

In additional exemplary embodiments, the single domain antibody comprises an amino acid sequence as set forth in SEQ ID NO:88.

In additional exemplary embodiments, the single domain antibody comprises an amino acid sequence as set forth in SEQ ID NO:89.

In additional exemplary embodiments, the single domain antibody comprises an amino acid sequence as set forth in SEQ ID NO:90.

In additional exemplary embodiments, the single domain antibody comprises an amino acid sequence as set forth in SEQ ID NO:91.

In additional exemplary embodiments, the single domain antibody comprises an amino acid sequence as set forth in SEQ ID NO:92.

In additional exemplary embodiments, the single domain antibody comprises an amino acid sequence as set forth in SEQ ID NO:93.

In additional exemplary embodiments, the single domain antibody comprises an amino acid sequence as set forth in SEQ ID NO:94.

In additional exemplary embodiments, the single domain antibody comprises an amino acid sequence as set forth in SEQ ID NO:95.

In additional exemplary embodiments, the single domain antibody comprises an amino acid sequence as set forth in SEQ ID NO:96.

In additional exemplary embodiments, the single domain antibody comprises an amino acid sequence as set forth in SEQ ID NO:97.

In additional exemplary embodiments, the single domain antibody comprises an amino acid sequence as set forth in SEQ ID NO:98.

In additional exemplary embodiments, the single domain antibody comprises an amino acid sequence as set forth in SEQ ID NO:99.

In additional exemplary embodiments, the single domain antibody comprises an amino acid sequence as set forth in SEQ ID NO:100.

In additional exemplary embodiments, the single domain antibody comprises an amino acid sequence as set forth in SEQ ID NO:101.

In additional exemplary embodiments, the single domain antibody comprises an amino acid sequence as set forth in SEQ ID NO:012.

In additional exemplary embodiments, the single domain antibody comprises an amino acid sequence as set forth in SEQ ID NO:103.

In additional exemplary embodiments, the single domain antibody comprises an amino acid sequence as set forth in SEQ ID NO:104.

In additional exemplary embodiments, the single domain antibody comprises an amino acid sequence as set forth in SEQ ID NO:105.

In additional exemplary embodiments, the single domain antibody comprises an amino acid sequence as set forth in SEQ ID NO:106.

In additional exemplary embodiments, the single domain antibody comprises an amino acid sequence as set forth in SEQ ID NO:107.

In additional exemplary embodiments, the single domain antibody comprises an amino acid sequence as set forth in SEQ ID NO:108.

In additional exemplary embodiments, the single domain antibody comprises an amino acid sequence as set forth in SEQ ID NO:109.

In additional exemplary embodiments, the single domain antibody comprises an amino acid sequence as set forth in SEQ ID NO:110.

In additional exemplary embodiments, the single domain antibody comprises an amino acid sequence as set forth in SEQ ID NO:111.

In additional exemplary embodiments, the single domain antibody comprises an amino acid sequence as set forth in SEQ ID NO:112.

In additional exemplary embodiments, the single domain antibody comprises an amino acid sequence as set forth in SEQ ID NO:113.

In additional exemplary embodiments, the single domain antibody comprises an amino acid sequence as set forth in SEQ ID NO:114.

In additional exemplary embodiments, the single domain antibody comprises an amino acid sequence as set forth in SEQ ID NO:115.

In additional exemplary embodiments, the single domain antibody comprises an amino acid sequence as set forth in SEQ ID NO:221.

In additional exemplary embodiments, the single domain antibody comprises an amino acid sequence as set forth in SEQ ID NO:222.

In additional exemplary embodiments, the single domain antibody comprises an amino acid sequence as set forth in SEQ ID NO:223.

In additional exemplary embodiments, the single domain antibody comprises an amino acid sequence as set forth in SEQ ID NO:224.

In additional exemplary embodiments, the single domain antibody comprises an amino acid sequence as set forth in SEQ ID NO:231.

In additional exemplary embodiments, the single domain antibody comprises an amino acid sequence as set forth in SEQ ID NO:232.

In additional exemplary embodiments, the single domain antibody comprises an amino acid sequence as set forth in SEQ ID NO:239.

In additional exemplary embodiments, the single domain antibody comprises an amino acid sequence as set forth in SEQ ID NO:240.

In additional exemplary embodiments, the single domain antibody comprises an amino acid sequence as set forth in SEQ ID NO:247.

In additional exemplary embodiments, the single domain antibody comprises an amino acid sequence as set forth in SEQ ID NO:295.

In additional exemplary embodiments, the single domain antibody comprises an amino acid sequence as set forth in SEQ ID NO:296.

In additional exemplary embodiments, the single domain antibody comprises an amino acid sequence as set forth in SEQ ID NO:297.

In additional exemplary embodiments, the single domain antibody comprises an amino acid sequence as set forth in SEQ ID NO:304.

In additional exemplary embodiments, the single domain antibody comprises an amino acid sequence as set forth in SEQ ID NO:311.

In additional exemplary embodiments, the single domain antibody comprises an amino acid sequence as set forth in SEQ ID NO:318.

In additional exemplary embodiments, the single domain antibody comprises an amino acid sequence as set forth in SEQ ID NO:325.

In additional exemplary embodiments, the single domain antibody comprises an amino acid sequence as set forth in SEQ ID NO:332.

In additional exemplary embodiments, the single domain antibody comprises an amino acid sequence as set forth in SEQ ID NO:339.

In additional exemplary embodiments, the single domain antibody comprises an amino acid sequence as set forth in SEQ ID NO:346.

In additional exemplary embodiments, the single domain antibody comprises an amino acid sequence as set forth in SEQ ID NO:353.

In additional exemplary embodiments, the single domain antibody comprises an amino acid sequence as set forth in SEQ ID NO:362.

In additional exemplary embodiments, the single domain antibody comprises an amino acid sequence as set forth in SEQ ID NO:363.

In additional exemplary embodiments, the single domain antibody comprises an amino acid sequence as set forth in SEQ ID NO:364.

In additional exemplary embodiments, the single domain antibody comprises an amino acid sequence as set forth in SEQ ID NO:365.

In additional exemplary embodiments, the single domain antibody comprises an amino acid sequence as set forth in SEQ ID NO:366.

In additional exemplary embodiments, the single domain antibody comprises an amino acid sequence as set forth in SEQ ID NO:367.

In additional exemplary embodiments, the single domain antibody comprises an amino acid sequence as set forth in SEQ ID NO:368.

In additional exemplary embodiments, the single domain antibody comprises an amino acid sequence as set forth in SEQ ID NO:369.

In additional exemplary embodiments, the single domain antibody comprises an amino acid sequence as set forth in SEQ ID NO:370.

In additional exemplary embodiments, the single domain antibody comprises an amino acid sequence as set forth in SEQ ID NO:371.

In additional exemplary embodiments, the single domain antibody comprises an amino acid sequence as set forth in SEQ ID NO:372.

In additional exemplary embodiments, the single domain antibody comprises an amino acid sequence as set forth in SEQ ID NO:373.

In additional exemplary embodiments, the single domain antibody comprises an amino acid sequence as set forth in SEQ ID NO:374.

In additional exemplary embodiments, the single domain antibody comprises an amino acid sequence as set forth in SEQ ID NO:375.

In additional exemplary embodiments, the single domain antibody comprises an amino acid sequence as set forth in SEQ ID NO:376.

In additional exemplary embodiments, the single domain antibody comprises an amino acid sequence as set forth in SEQ ID NO:377.

In additional exemplary embodiments, the single domain antibody comprises an amino acid sequence as set forth in SEQ ID NO:378.

In additional exemplary embodiments, the single domain antibody comprises an amino acid sequence as set forth in SEQ ID NO:379.

In additional exemplary embodiments, the single domain antibody comprises an amino acid sequence as set forth in SEQ ID NO:380.

In additional exemplary embodiments, the single domain antibody comprises an amino acid sequence as set forth in SEQ ID NO:381.

In additional exemplary embodiments, the single domain antibody comprises an amino acid sequence as set forth in SEQ ID NO:382.

In additional exemplary embodiments, the single domain antibody comprises an amino acid sequence as set forth in SEQ ID NO:383.

In additional exemplary embodiments, the single domain antibody comprises an amino acid sequence as set forth in SEQ ID NO:384.

In additional exemplary embodiments, the single domain antibody comprises an amino acid sequence as set forth in SEQ ID NO:385.

In additional exemplary embodiments, the single domain antibody comprises an amino acid sequence as set forth in SEQ ID NO:386.

In additional exemplary embodiments, the single domain antibody comprises an amino acid sequence as set forth in SEQ ID NO:387.

In additional exemplary embodiments, the single domain antibody comprises an amino acid sequence as set forth in SEQ ID NO:388.

In additional exemplary embodiments, the single domain antibody comprises an amino acid sequence as set forth in SEQ ID NO:389.

In additional exemplary embodiments, the single domain antibody comprises an amino acid sequence as set forth in SEQ ID NO:390.

In additional exemplary embodiments, the single domain antibody comprises an amino acid sequence as set forth in SEQ ID NO:397.

In additional exemplary embodiments, the single domain antibody comprises an amino acid sequence as set forth in SEQ ID NO:398.

In additional exemplary embodiments, the single domain antibody comprises an amino acid sequence as set forth in SEQ ID NO:405.

In additional exemplary embodiments, the single domain antibody comprises an amino acid sequence as set forth in SEQ ID NO:412.

In additional exemplary embodiments, the single domain antibody comprises an amino acid sequence as set forth in SEQ ID NO:419.

In additional exemplary embodiments, the single domain antibody comprises an amino acid sequence as set forth in SEQ ID NO:426.

In additional exemplary embodiments, the single domain antibody comprises an amino acid sequence as set forth in SEQ ID NO:433.

In additional exemplary embodiments, the single domain antibody comprises an amino acid sequence as set forth in SEQ ID NO:440.

In additional exemplary embodiments, the single domain antibody comprises an amino acid sequence as set forth in SEQ ID NO:447.

In additional exemplary embodiments, the single domain antibody comprises an amino acid sequence as set forth in SEQ ID NO:454.

In additional exemplary embodiments, the single domain antibody comprises an amino acid sequence as set forth in SEQ ID NO:461.

In additional exemplary embodiments, the single domain antibody comprises an amino acid sequence as set forth in SEQ ID NO:468.

In additional exemplary embodiments, the single domain antibody comprises an amino acid sequence as set forth in SEQ ID NO:469.

In additional exemplary embodiments, the single domain antibody comprises an amino acid sequence as set forth in SEQ ID NO:470.

In additional exemplary embodiments, the single domain antibody comprises an amino acid sequence as set forth in SEQ ID NO:471.

In additional exemplary embodiments, the single domain antibody comprises an amino acid sequence as set forth in SEQ ID NO:472.

In additional exemplary embodiments, the single domain antibody comprises an amino acid sequence as set forth in SEQ ID NO:473.

In additional exemplary embodiments, the single domain antibody comprises an amino acid sequence as set forth in SEQ ID NO:491.

In additional exemplary embodiments, the single domain antibody comprises an amino acid sequence as set forth in SEQ ID NO:493.

In additional exemplary embodiments, the single domain antibody comprises an amino acid sequence as set forth in SEQ ID NO:496.

In additional exemplary embodiments, the single domain antibody comprises an amino acid sequence as set forth in SEQ ID NO:502.

In additional exemplary embodiments, the single domain antibody comprises an amino acid sequence as set forth in SEQ ID NO:503.

In exemplary embodiments, single domain antibodies comprising the CDRs or heavy chain variable regions set forth in SEQ ID NO:221, 222, 223, 224, 231, 232, 239, 240, 247, 325, 332, 339, 346, 362, 363, 364, 365, 366, 367, 368, 369, 370, 371, 296, 297, 372, 373, 374, 375, 376, 377, 378, 379, 380, 381, 382, 383, 384, 385, 386, 387, 388, 389, 390, 397, 398, 405, 412, 419, 426, 433, 440, 447, 454, 461, 468, 318 or humanized versions thereof are particularly selected.

In another exemplary embodiment, the single domain antibody or antigen binding fragment thereof comprises the heavy chain of the single domain antibody disclosed herein.

In exemplary embodiments, the single domain antibody comprises an amino acid sequence as set forth in SEQ ID NO:29.

In exemplary embodiments, the single domain antibody comprises an amino acid sequence as set forth in SEQ ID NO:30.

In yet other exemplary embodiments, the single domain antibody comprises an amino acid sequence as set forth in SEQ ID NO:31.

In further exemplary embodiments, the single domain antibody comprises an amino acid sequence as set forth in SEQ ID NO:32.

In yet further exemplary embodiments, the single domain antibody comprises an amino acid sequence as set forth in SEQ ID NO:33.

In other exemplary embodiments, the single domain antibody comprises an amino acid sequence as set forth in SEQ ID NO:34.

In other exemplary embodiments, the single domain antibody comprises an amino acid sequence as set forth in SEQ ID NO:35.

In other exemplary embodiments, the single domain antibody comprises an amino acid sequence as set forth in SEQ ID NO:44.

In other exemplary embodiments, the single domain antibody comprises an amino acid sequence as set forth in SEQ ID NO:45.

In other exemplary embodiments, the single domain antibody comprises an amino acid sequence as set forth in SEQ ID NO:482.

In other exemplary embodiments, the single domain antibody comprises an amino acid sequence as set forth in SEQ ID NO:483.

In other exemplary embodiments, the single domain antibody comprises an amino acid sequence as set forth in SEQ ID NO:484.

In other exemplary embodiments, the single domain antibody comprises an amino acid sequence as set forth in SEQ ID NO:494.

In other exemplary embodiments, the single domain antibody comprises an amino acid sequence as set forth in SEQ ID NO:497.

In other exemplary embodiments, the single domain antibody comprises an amino acid sequence as set forth in SEQ ID NO:504.

In some instances, the binding agent of the present disclosure does not comprise the amino acid sequence set forth in any one of SEQ ID Nos:22-28, 71-115, 143 to 146.

In some instances, the binding agent of the present disclosure does not comprise the amino acid sequence set forth in SEQ ID NO:295.

In some instances, the binding agent of the present disclosure does not comprise the amino acid sequence set forth in SEQ ID NO:304.

In some instances, the binding agent of the present disclosure does not comprise the amino acid sequence set forth in SEQ ID NO:311.

In some instances, the binding agent of the present disclosure does not comprise the amino acid sequence set forth in SEQ ID NO:353.

In some instances, the binding agent of the present disclosure does not comprise the amino acid sequence set forth in SEQ ID NO:362.

In some instances, the binding agent of the present disclosure may comprise the amino acid sequence set forth in any one of SEQ ID Nos:22-28, 71-115, 143 to 146 provided that it also comprises an amino acid sequence as set forth in any one of SEQ ID NOs:221-224, SEQ ID NO:231, SEQ ID NO:232, SEQ ID NO:239, SEQ ID NO:240, SEQ ID NO:247, SEQ ID NO:304, SEQ ID NO:295, SEQ ID NO:296, SEQ ID NO:297, SEQ ID NO:311, SEQ ID NO:318, SEQ ID NO:325, SEQ ID NO:332, SEQ ID NO:339, SEQ ID NO:346, SEQ ID NO:353, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503.

In accordance with the present disclosure the single domain antibody may comprise the amino acid sequence combination of Table 1A and a dimerization domain.

In some embodiments, the dimerization domain may comprise SEQ ID NO:38 or a variant thereof. In other embodiments, the dimerization domain may comprise SEQ ID NO:43 or a variant thereof. The dimerization domain may also comprise SEQ ID NO:37 or SEQ ID NO:179 or a variant thereof in combination with either SEQ ID NO:36 or SEQ ID NO:42 or a variant thereof.

In yet embodiments, the dimerization domain may comprise SEQ ID NO:212 or a variant thereof. In yet embodiments, the dimerization domain may comprise SEQ ID NO:506 or a variant thereof. In further embodiments, the dimerization domain may comprise SEQ ID NO:214 or a variant thereof.

Accordingly, in exemplary embodiments the single domain antibody may comprise an amino acid sequence as set forth in Table 1B.

TABLE 1B

| CDRH1; CDRH2; | Exemplary heavy chain variable | |
|---|---|---|
| CDRH3 | region backbone | Exemplary dimerization domain |
| SEQ ID NO: 52; SEQ ID | SEQ ID NOs: 248, 249, 250, 251, | a) SEQ ID NO: 38, |
| NO: 53; SEQ ID NO: 54 | 252, 253, 254, 255, 256, 257, 258, | b) SEQ ID NO: 37 and 38, |
| or | 259, 260, 261, 262, 263, 264, 265, | c) SEQ ID NO: 36 (or 481), 37 |
| SEQ ID NO: 218; SEQ | 266, 267, 268, 269, 270, or 271 | and 38 or, |
| ID NO: 219; SEQ ID | | d) SEQ ID NO: 212 or 506 |
| NO: 220 | | |
| SEQ ID NO: 52; SEQ ID | SEQ ID NOs: 272, 273, 274, 275, | a) SEQ ID NO: 38, |
| NO: 53; SEQ ID NO: 54 | 276, 277, 278, 279, 280, 281, 282, | b) SEQ ID NO: 37 and 38, |
| or | 283, 284, 285, 286, 287, 288, 289, | c) SEQ ID NO: 36 (or 481), 37 |
| SEQ ID NO: 218; SEQ | 290, 291, 292, 293 or 294. | and 38 or, |
| ID NO: 219; SEQ ID | | d) SEQ ID NO: 212 or 506 |
| NO: 220 | | |
| SEQ ID NO 55; SEQ ID | SEQ ID NOs: 248, 249, 250, 251, | a) SEQ ID NO: 38, |
| NO: 47; SEQ ID NO: 48 | 252, 253, 254, 255, 256, 257, 258, | b) SEQ ID NO: 37 and 38, |
| | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO 55; SEQ ID | SEQ ID NOs: 272, 273, 274, 275, | a) SEQ ID NO: 38, |
| NO: 47; SEQ ID NO: 48 | 276, 277, 278, 279, 280, 281, 282, | b) SEQ ID NO: 37 and 38, |
| | 283, 284, 285, 286, 287, 288, 289, | c) SEQ ID NO: 36 (or 481), 37 |
| | 290, 291, 292, 293 or 294. | and 38 or, |
| | | d) SEQ ID NO: 212 or 506 |
| SEQ ID NO: 56; SEQ ID | SEQ ID NOs: 248, 249, 250, 251, | a) SEQ ID NO: 38, |
| NO: 57; SEQ ID NO: 58 | 252, 253, 254, 255, 256, 257, 258, | b) SEQ ID NO: 37 and 38, |
| | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 56; SEQ ID | SEQ ID NOs: 272, 273, 274, 275, | a) SEQ ID NO: 38, |
| NO: 57; SEQ ID NO: 58 | 276, 277, 278, 279, 280, 281, 282, | b) SEQ ID NO: 37 and 38, |
| | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 59; SEQ ID | SEQ ID NOs: 248, 249, 250, 251, | a) SEQ ID NO: 38, |
| NO: 60; SEQ ID NO: 61 | 252, 253, 254, 255, 256, 257, 258, | b) SEQ ID NO: 37 and 38, |
| or | 259, 260, 261, 262, 263, 264, 265, | c) SEQ ID NO: 36 (or 481), 37 |
| SEQ ID NO: 301; SEQ | 266, 267, 268, 269, 270, or 271 | and 38 or, |
| ID NO: 302; SEQ ID | | d) SEQ ID NO: 212 or 506 |
| NO: 303 | | |
| SEQ ID NO: 59; SEQ ID | SEQ ID NOs: 272, 273, 274, 275, | a) SEQ ID NO: 38, |
| NO: 60; SEQ ID NO: 61 | 276, 277, 278, 279, 280, 281, 282, | b) SEQ ID NO: 37 and 38, |
| or | 283, 284, 285, 286, 287, 288, 289, | c) SEQ ID NO: 36 (or 481), 37 |
| SEQ ID NO: 301; SEQ | 290, 291, 292, 293 or 294 | and 38 or, |
| ID NO: 302; SEQ ID | | d) SEQ ID NO: 212 or 506 |
| NO: 303 | | |
| SEQ ID NO: 62; SEQ ID | SEQ ID NOs: 248, 249, 250, 251, | a) SEQ ID NO: 38, |
| NO: 50; SEQ ID NO: 51 | 252, 253, 254, 255, 256, 257, 258, | b) SEQ ID NO: 37 and 38, |
| | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 62; SEQ ID | SEQ ID NOs: 272, 273, 274, 275, | a) SEQ ID NO: 38, |
| NO: 50; SEQ ID NO: 51 | 276, 277, 278, 279, 280, 281, 282, | b) SEQ ID NO: 37 and 38, |
| | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 228; SEQ | SEQ ID NOs: 248, 249, 250, 251, | a) SEQ ID NO: 38, |
| ID NO: 229; SEQ ID | 252, 253, 254, 255, 256, 257, 258, | b) SEQ ID NO: 37 and 38, |
| NO: 230 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 228; SEQ | SEQ ID NOs: 272, 273, 274, 275, | a) SEQ ID NO: 38, |
| ID NO: 229; SEQ ID | 276, 277, 278, 279, 280, 281, 282, | b) SEQ ID NO: 37 and 38, |
| NO: 230 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 236; SEQ | SEQ ID NOs: 248, 249, 250, 251, | a) SEQ ID NO: 38, |
| ID NO: 237; SEQ ID | 252, 253, 254, 255, 256, 257, 258, | b) SEQ ID NO: 37 and 38, |
| NO: 238 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 236; SEQ | SEQ ID NOs: 272, 273, 274, 275, | a) SEQ ID NO: 38, |
| ID NO: 237; SEQ ID | 276, 277, 278, 279, 280, 281, 282, | b) SEQ ID NO: 37 and 38, |
| NO: 238 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 244; SEQ | SEQ ID NOs: 248, 249, 250, 251, | a) SEQ ID NO: 38, |
| ID NO: 245; SEQ ID | 252, 253, 254, 255, 256, 257, 258, | b) SEQ ID NO: 37 and 38, |
| NO: 246 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 244; SEQ | SEQ ID NOs: 272, 273, 274, 275, | a) SEQ ID NO: 38, |
| ID NO: 245; SEQ ID | 276, 277, 278, 279, 280, 281, 282, | b) SEQ ID NO: 37 and 38, |
| NO: 246 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 308; SEQ | SEQ ID NOs: 248, 249, 250, 251, | a) SEQ ID NO: 38, |
| ID NO: 309; SEQ ID | 252, 253, 254, 255, 256, 257, 258, | b) SEQ ID NO: 37 and 38, |
| NO: 310 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 308; SEQ | SEQ ID NOs: 272, 273, 274, 275, | a) SEQ ID NO: 38, |
| ID NO: 309; SEQ ID | 276, 277, 278, 279, 280, 281, 282, | b) SEQ ID NO: 37 and 38, |
| NO: 310 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 315; SEQ | SEQ ID NOs: 248, 249, 250, 251, | a) SEQ ID NO: 38, |
| ID NO: 316; SEQ ID | 252, 253, 254, 255, 256, 257, 258, | b) SEQ ID NO: 37 and 38, |
| NO: 317 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 315; SEQ | SEQ ID NOs: 272, 273, 274, 275, | a) SEQ ID NO: 38, |
| ID NO: 316; SEQ ID | 276, 277, 278, 279, 280, 281, 282, | b) SEQ ID NO: 37 and 38, |
| NO: 317 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 322; SEQ | SEQ ID NOs: 248, 249, 250, 251, | a) SEQ ID NO: 38, |
| ID NO: 323; SEQ ID | 252, 253, 254, 255, 256, 257, 258, | b) SEQ ID NO: 37 and 38, |
| NO: 324 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 322; SEQ | SEQ ID NOs: 272, 273, 274, 275, | a) SEQ ID NO: 38, |
| ID NO: 323; SEQ ID | 276, 277, 278, 279, 280, 281, 282, | b) SEQ ID NO: 37 and 38, |
| NO: 324 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 329; SEQ | SEQ ID NOs: 248, 249, 250, 251, | a) SEQ ID NO: 38, |
| ID NO: 330; SEQ ID | 252, 253, 254, 255, 256, 257, 258, | b) SEQ ID NO: 37 and 38, |
| NO: 331 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 329; SEQ | SEQ ID NOs: 272, 273, 274, 275, | a) SEQ ID NO: 38, |
| ID NO: 330; SEQ ID | 276, 277, 278, 279, 280, 281, 282, | b) SEQ ID NO: 37 and 38, |
| NO: 331 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 336; SEQ | SEQ ID NOs: 248, 249, 250, 251, | a) SEQ ID NO: 38, |
| ID NO: 337; SEQ ID | 252, 253, 254, 255, 256, 257, 258, | b) SEQ ID NO: 37 and 38, |
| NO: 338 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 336; SEQ | SEQ ID NOs: 272, 273, 274, 275, | a) SEQ ID NO: 38, |
| ID NO: 337; SEQ ID | 276, 277, 278, 279, 280, 281, 282, | b) SEQ ID NO: 37 and 38, |
| NO: 338 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 343; SEQ | SEQ ID NOs: 248, 249, 250, 251, | a) SEQ ID NO: 38, |
| ID NO: 344; SEQ ID | 252, 253, 254, 255, 256, 257, 258, | b) SEQ ID NO: 37 and 38, |
| NO: 345 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 343; SEQ | SEQ ID NOs: 272, 273, 274, 275, | a) SEQ ID NO: 38, |
| ID NO: 344; SEQ ID | 276, 277, 278, 279, 280, 281, 282, | b) SEQ ID NO: 37 and 38, |
| NO: 345 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 350; SEQ | SEQ ID NOs: 248, 249, 250, 251, | a) SEQ ID NO: 38, |
| ID NO: 351; SEQ ID | 252, 253, 254, 255, 256, 257, 258, | b) SEQ ID NO: 37 and 38, |
| NO: 352 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 350; SEQ | SEQ ID NOs: 272, 273, 274, 275, | a) SEQ ID NO: 38, |
| ID NO: 351; SEQ ID | 276, 277, 278, 279, 280, 281, 282, | b) SEQ ID NO: 37 and 38, |
| NO: 352 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 394; SEQ | SEQ ID NOs: 248, 249, 250, 251, | a) SEQ ID NO: 38, |
| ID NO: 395; SEQ ID | 252, 253, 254, 255, 256, 257, 258, | b) SEQ ID NO: 37 and 38, |
| NO: 396 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 394; SEQ | SEQ ID NOs: 272, 273, 274, 275, | a) SEQ ID NO: 38, |
| ID NO: 395; SEQ ID | 276, 277, 278, 279, 280, 281, 282, | b) SEQ ID NO: 37 and 38, |
| NO: 396 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 402; SEQ | SEQ ID NOs: 248, 249, 250, 251, | a) SEQ ID NO: 38, |
| ID NO: 403; SEQ ID | 252, 253, 254, 255, 256, 257, 258, | b) SEQ ID NO: 37 and 38, |
| NO: 404 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 402; SEQ | SEQ ID NOs: 272, 273, 274, 275, | a) SEQ ID NO: 38, |
| ID NO: 403; SEQ ID | 276, 277, 278, 279, 280, 281, 282, | b) SEQ ID NO: 37 and 38, |
| NO: 404 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 409; SEQ | SEQ ID NOs: 248, 249, 250, 251, | a) SEQ ID NO: 38, |
| ID NO: 410; SEQ ID | 252, 253, 254, 255, 256, 257, 258, | b) SEQ ID NO: 37 and 38, |
| NO: 411 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 409; SEQ | SEQ ID NOs: 272, 273, 274, 275, | a) SEQ ID NO: 38, |
| ID NO: 410; SEQ ID | 276, 277, 278, 279, 280, 281, 282, | b) SEQ ID NO: 37 and 38, |
| NO: 411 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 416; SEQ | SEQ ID NOs: 248, 249, 250, 251, | a) SEQ ID NO: 38, |
| ID NO: 417; SEQ ID | 252, 253, 254, 255, 256, 257, 258, | b) SEQ ID NO: 37 and 38, |
| NO: 418 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 416; SEQ | SEQ ID NOs: 272, 273, 274, 275, | a) SEQ ID NO: 38, |
| ID NO: 417; SEQ ID | 276, 277, 278, 279, 280, 281, 282, | b) SEQ ID NO: 37 and 38, |
| NO: 418 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 423; SEQ | SEQ ID NOs: 248, 249, 250, 251, | a) SEQ ID NO: 38, |
| ID NO: 424; SEQ ID | 252, 253, 254, 255, 256, 257, 258, | b) SEQ ID NO: 37 and 38, |
| NO: 425 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 423; SEQ | SEQ ID NOs: 272, 273, 274, 275, | a) SEQ ID NO: 38, |
| ID NO: 424; SEQ ID | 276, 277, 278, 279, 280, 281, 282, | b) SEQ ID NO: 37 and 38, |
| NO: 425 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 430; SEQ | SEQ ID NOs: 248, 249, 250, 251, | a) SEQ ID NO: 38, |
| ID NO: 431; SEQ ID | 252, 253, 254, 255, 256, 257, 258, | b) SEQ ID NO: 37 and 38, |
| NO: 432 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 430; SEQ | SEQ ID NOs: 272, 273, 274, 275, | a) SEQ ID NO: 38, |
| ID NO: 431; SEQ ID | 276, 277, 278, 279, 280, 281, 282, | b) SEQ ID NO: 37 and 38, |
| NO: 432 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 437; SEQ | SEQ ID NOs: 248, 249, 250, 251, | a) SEQ ID NO: 38, |
| ID NO: 438; SEQ ID | 252, 253, 254, 255, 256, 257, 258, | b) SEQ ID NO: 37 and 38, |
| NO: 439 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 437; SEQ | SEQ ID NOs: 272, 273, 274, 275, | a) SEQ ID NO: 38, |
| ID NO: 438; SEQ ID | 276, 277, 278, 279, 280, 281, 282, | b) SEQ ID NO: 37 and 38, |
| NO: 439 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 444; SEQ | SEQ ID NOs: 248, 249, 250, 251, | a) SEQ ID NO: 38, |
| ID NO: 445; SEQ ID | 252, 253, 254, 255, 256, 257, 258, | b) SEQ ID NO: 37 and 38, |
| NO: 446 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 444; SEQ | SEQ ID NOs: 272, 273, 274, 275, | a) SEQ ID NO: 38, |
| ID NO: 445; SEQ ID | 276, 277, 278, 279, 280, 281, 282, | b) SEQ ID NO: 37 and 38, |
| NO: 446 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 451; SEQ | SEQ ID NOs: 248, 249, 250, 251, | a) SEQ ID NO: 38, |
| ID NO: 452; SEQ ID | 252, 253, 254, 255, 256, 257, 258, | b) SEQ ID NO: 37 and 38, |
| NO: 453 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 451; SEQ | SEQ ID NOs: 272, 273, 274, 275, | a) SEQ ID NO: 38, |
| ID NO: 452; SEQ ID | 276, 277, 278, 279, 280, 281, 282, | b) SEQ ID NO: 37 and 38, |
| NO: 453 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 458; SEQ | SEQ ID NOs: 248, 249, 250, 251, | a) SEQ ID NO: 38, |
| ID NO: 459; SEQ ID | 252, 253, 254, 255, 256, 257, 258, | b) SEQ ID NO: 37 and 38, |
| NO: 460 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 458; SEQ | SEQ ID NOs: 272, 273, 274, 275, | a) SEQ ID NO: 38, |
| ID NO: 459; SEQ ID | 276, 277, 278, 279, 280, 281, 282, | b) SEQ ID NO: 37 and 38, |
| NO: 460 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 465; SEQ | SEQ ID NOs: 248, 249, 250, 251, | a) SEQ ID NO: 38, |
| ID NO: 466; SEQ ID | 252, 253, 254, 255, 256, 257, 258, | b) SEQ ID NO: 37 and 38, |
| NO: 467 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 465; SEQ | SEQ ID NOs: 272, 273, 274, 275, | a) SEQ ID NO: 38, |
| ID NO: 466; SEQ ID | 276, 277, 278, 279, 280, 281, 282, | b) SEQ ID NO: 37 and 38, |
| NO: 467 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 488; SEQ | SEQ ID NOs: 248, 249, 250, 251, | a) SEQ ID NO: 38, |
| ID NO: 489; SEQ ID | 252, 253, 254, 255, 256, 257, 258, | b) SEQ ID NO: 37 and 38, |
| NO: 490 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 488; SEQ | SEQ ID NOs: 272, 273, 274, 275, | a) SEQ ID NO: 38, |
| ID NO: 489; SEQ ID | 276, 277, 278, 279, 280, 281, 282, | b) SEQ ID NO: 37 and 38, |
| NO: 490 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |
| SEQ ID NO: 500; SEQ | SEQ ID NOs: 248, 249, 250, 251, | a) SEQ ID NO: 38, |
| ID NO: 489; SEQ ID | 252, 253, 254, 255, 256, 257, 258, | b) SEQ ID NO: 37 and 38, |
| NO: 501 | 259, 260, 261, 262, 263, 264, 265, | c) SEQ ID NO: 36 (or 481), 37 |
| | 266, 267, 268, 269, 270, or 271 | and 38 or, |
| | | d) SEQ ID NO: 212 or 506 |
| SEQ ID NO: 500; SEQ | SEQ ID NOs: 272, 273, 274, 275, | a) SEQ ID NO: 38, |
| ID NO: 489; SEQ ID | 276, 277, 278, 279, 280, 281, 282, | b) SEQ ID NO: 37 and 38, |
| NO: 501 | 283, 284, 285, 286, 287, 288, 289, | c) SEQ ID NO: 36 (or 481), 37 |
| | 290, 291, 292, 293 or 294 | and 38 or, |
| | | d) SEQ ID NO: 212 or 506 |

Binding Agents Comprising Antigen Binding Domain(s) of ssdAbs

Binding agents of the present disclosure comprise one or more antigen binding domain(s) of an antibody or antigen binding fragment thereof that bind to TROP2. For example, the binding agents of the present disclosure comprise one or more antigen binding domain(s) of a single domain antibody disclosed herein. For example, the binding agent may comprise the 3 CDRs of a single domain disclosed herein. In another example, the binding agent may comprise the variable region of the single domain disclosed herein or a portion thereof.

It is to be understood herein that the following represent exemplary and non-limiting embodiments of binding agents comprising antigen binding domain(s) of single domain antibodies.

In some embodiments, the binding agent of the present disclosure, comprises one or more antigen binding domains that specifically bind to trophoblast cell surface antigen-2 (TROP2). In some embodiments, the binding agent of the present disclosure, comprises one or more antigen binding domains that bind to a different target than TROP2.

Exemplary embodiments of binding agents of the present disclosure include those that comprise one or more antigen binding domains that specifically bind to at least one epitope comprising amino acids of the extracellular domain (ECD) of TROP2. Other exemplary embodiments of binding agents of the present disclosure include those that comprise one or more antigen binding domains that specifically bind to at least one epitope comprising amino acids of the cysteine rich domain of TROP2. In some embodiments, the binding agent comprise one or more antigen binding domains that specifically bind to a TROP2 antigen disclosed herein.

In an embodiment, the binding agent of the present disclosure comprises one or more antigen binding domains that specifically bind to human TROP2. In another embodiment, the binding agent of the present disclosure comprises one or more antigen binding domains that specifically bind to cynomolgus TROP2. In another embodiment, the binding agent of the present disclosure comprises one or more antigen binding domains that specifically bind to Rhesus TROP2.

More particularly, the binding agent of the present disclosure comprises one or more antigen binding domains that specifically bind to the ECD or the CRD of human TROP2.

In other embodiments, the binding agents of the present disclosure comprise one or more antigen binding domain(s) of a native antibody (CDRs, and/or VL and VH) that binds to TROP2.

The CDRs or variable regions (VL and VH) of antibodies and antigen binding fragments thereof may be expressed as a single polypeptide chain to generate single chain Fv (scFV) molecules and variants. Numerous formats are contemplated as indicated below.

As well, the CDRs or variable region of multiple single domain antibodies may be expressed as a single polypeptide chain thereby providing multivalence and/or multispecificity to the binding agent. Moreover, multiple polypeptide chains may be assembled to increase the diversity or avidity of interactions with TROP2. Numerous formats are contemplated as indicated below.

Accordingly, antigen binding fragments or antigen binding domain(s) of the single domain antibodies disclosed herein may be used to generate binding agents that are monospecific, multispecific, monovalent or multivalent.

For example, the variable region or CDRs of a single domain antibody can be fused with a Fc, CH3 or CH2-CH3 domain of an antibody to allow homodimerization thereby generating a multivalent binding agent.

Alternatively, multiple copies of the variable region or CDRs of a single domain antibody can be fused in tandem with required spacers or linkers to generate a multivalent and monospecific binding agent.

For example, the variable regions or CDRs of two single domain antibodies can be fused with a Fc, CH3 or CH2-CH3 domain to allow heterodimerization thereby generating a multivalent and multispecific binding agent.

Alternatively, the variable regions or CDRs of two or more single domain antibodies may be fused in tandem with required spacers or linkers to generate a multivalent and multispecific binding agent.

Binding agents of the present disclosure may have a format of an antibody or antigen binding fragment thereof, an antibody-like molecule (native antibody fused with a single domain antibody or with an antigen binding domain of a sdAb, Fc-, CH3- fusions and the like), a fusion with protein scaffolds, immune cell modulating agents and the like.

Binding agents also include immune cell modulating agents such as for example, dual-affinity retargeting molecules (DARTs), chimeric antigen receptors (CAR) constructs, bispecific T cell engagers construct (BiTEs), bispecific killer cell engagers (BiKEs), trispecific killer cell engagers (TriKEs) containing scFvs or VHHs.

Binding agents also encompass fusion with protein scaffolds, including ankyrin repeat proteins, Z-domain of Staphylococcus protein-A, Type-III fibronetin, knottin and the like.

Exemplary embodiments of binding agents include monospecific-, bispecifics (symmetric or asymmetric) trispecific-, or multispecific antibodies as well as monovalent, bivalent, trivalent or multivalent antibodies, single chain FVs (scFVs) and derivatives such diabody, triabody, tetrabody, tandem di-scFvs, tandem tri-scFvs, scFV-Fc, minibody (scFV-CH3), tandem diabody, di-diabody, bibody and the like, VH or VHHs and derivatives such as, tandem bispecific or multispecific VHH, bivalent VHH-Fc fusions, VHH-hinge-CH2-CH3 fusions, bivalent CH3 fusions, VHH pentabody, decabody and the like.

A single chain Fv molecule (scFv) comprises a VL domain and a VH domain. The VL and VH domains associate to form a target binding site. These two domains are further covalently linked by a peptide linker (L). A scFv molecule is denoted as either VL-L-VH if the VL domain is the N-terminal part of the scFv molecule, or as VH-L-VL if the VH domain is the N-terminal part of the scFv molecule. Methods for making scFv molecules and designing suitable peptide linkers are described in U.S. Pat. Nos. 4,704,692, 4,946,778, R. Raag and M. Whitlow, “Single Chain Fvs.” FASEB Vol 9:73-80 (1995) and R. E. Bird and B. W. Walker, Single Chain Antibody Variable Regions, TIBTECH, Vol 9: 132-137 (1991).

In some embodiments, the binding agents of the present disclosure may have a format as disclosed in PCT/CA2020/051753 filed on Dec. 18, 2020 and published on Jun. 24, 2021 under No. WO2021/119832A1, the entire content of which is incorporated herein by reference) such as for example, as formula Ia, formula Ib, formula Ic, formula II, formula III, formula IV, formula V, formula VI, formula VII or formula VIII and the like or formula I, formula II, formula III, formula IIIa and formula IIIb, formula IV, formula V, formula VI, formula VII or formula VIII disclosed herein.

The binding agents of the present disclosure may be formed by the assembly of two polypeptide chains having the same configuration (with same or different amino acid sequence) or having different configurations where the same or different configurations include the configuration set forth in formula I, formula II, formula III, formula IIIa and formula IIIb, formula IV, formula V, formula VI, formula VII or formula VIII disclosed herein.

In some embodiments, the binding agents of the present disclosure is monospecific. In some embodiments, the binding agents of the present disclosure is multispecific.

The binding agents of the present disclosure (including antibodies or antigen binding fragments thereof) may be monospecific, multispecific, monovalent or multivalent.

A binding agent that comprises two or more antigen binding domains of a single domain antibody is considered multivalent. A multivalent single domain antibody can be monospecific (if all antigen binding domains bind to exactly the same epitope) or multispecific (if the antigen binding domains bind to different epitopes on same target or to different epitopes or targets). A binding agent that comprises only one antigen binding domain is considered monovalent.

Production of binding agents such as antibodies or antigen binding fragments, may involve cloning procedures and recombinant expression. The antigen binding domain sequence (e.g., sequence of the variable light chain and variable heavy chain of an intact antibody or the sequence of the variable heavy chain of a single domain antibody) may be obtained by a variety of molecular cloning procedures, such as RT-PCR, 5′-RACE, and cDNA library screening. The sequence of interest may be cloned into an expression vector(s), the expression vector(s) transfected into cells and the binding agent isolated and/or purified. In the case of an intact antibody, the light chain and the heavy chain can be cloned into a single expression vector or into two separate expression vectors. Co-transfection of the separate expression vectors is necessary for the intact antibody to assemble properly. In the case of a single domain antibody, a single expression vector (expressing the heavy chain) is sufficient.

Monospecific binding agents encompass binding agents that are specific for a single epitope of a given antigen.

An exemplary embodiment of a monospecific binding agent includes a binding agent that comprise one antigen binding fragment of a single domain antibody. Another exemplary embodiment of a monospecific binding agent includes a binding agent that comprise more than one antigen binding fragments of a single domain antibody, but the antigen binding fragments have identical CDRs and framework regions. Yet another exemplary embodiment of a binding agent includes a binding agent that comprise more than one antigen binding fragments of single domain antibodies, but the variable regions have identical CDRs and different framework regions. A further exemplary embodiment of a monospecific binding agent includes a binding agent that comprise antigen binding fragments of single domain antibodies that differ in the amino acid sequence of one or more of their CDRs (e.g., conservative substitution in one or more CDRs) without affecting their ability to bind to the same antigen or epitope.

Multispecific binding agents encompass binding agents that are specific for more than one epitope (of the same antigen or different antigens) or to more than one antigen. For example, a multispecific polypeptide chain or binding agent may thus have more than one variable region of a single domain antibody, at least two of which bind to different antigens or epitopes.

The binding agents of the present disclosure may thus be bispecific, trispecific, tetraspecific, pentaspecific, hexaspecific etc. In some embodiments, each variable region may be specific for a given antigen. In some embodiments, two or more antigen binding fragments of a given binding agent may be specific for the same or different antigens. In some embodiments, three or more antigen binding fragments of a given binding agent may be specific for the same or different antigens. In some embodiments, four or more antigen binding fragments of a given binding agent may be specific for the same or different antigens. In some embodiments, five or more antigen binding fragments of a given binding agent may be specific for the same or different antigens. In some embodiments, six or more antigen binding fragments of a given binding agent may be specific for the same or different antigens. The specificity may depend on the number of antigen binding fragments present in a given binding agent.

Other exemplary non-limiting embodiments of multispecific binding agents include those having two antigen binding fragments of different specificities. Yet other exemplary non-limiting embodiments of multispecific binding agents include those having more than two antigen binding fragments that bind to two different antigens, proteins or to two different epitopes on the same antigens or proteins.

In some embodiments, the binding agent of the present disclosure is monovalent.

In some embodiments, the binding agent of the present disclosure is multivalent.

Exemplary non-limiting embodiments of multivalent binding agents include binding agents composed of multivalent polypeptide chains. Other non-limiting exemplary embodiments of multivalent binding agents include binding agents composed of more than one monovalent polypeptide chain.

In accordance with the present disclosure, a bispecific binding agent may be bivalent or multivalent depending on the number of variable regions that it contains. Exemplary non-limiting embodiments of bispecific binding agents include those comprising two identical bispecific polypeptide chains that form a dimer.

Exemplary and non-limiting embodiments of bispecific binding agents are provided herein.

In some embodiments, the bispecific binding agent comprises an antigen binding domain that binds to TROP2 and comprises formula II (see PCT/CA2020/051753 filed on Dec. 18, 2020, and published on Jun. 24, 2021, under No. WO2021/119832A1 the entire content of which is herein incorporated by reference).

In other embodiments, the binding agent comprises a polypeptide chain that comprises in a N- to C-terminal fashion, a) an antigen binding domain of a single domain antibody, b) a linker, c) a dimerization domain, d) a linker and e) an antigen binding domain of a single domain antibody, at least one of the antigen binding domain of a) or e) is an antigen binding domain that binds to TROP2.

In some embodiments, the antigen binding domain of a) and the antigen binding domain of e) are different.

In some embodiments, the antigen binding domain of a) and the antigen binding domain of e) are the same.

In some embodiments, the linker of b) and the linker of d) are the same.

In some embodiments, the linker of b) and the linker of d) are different.

In an exemplary embodiment, the antigen binding domain of a) comprises an antigen binding domain of a single domain antibody that specifically binds to human TROP2.

In another exemplary embodiment, the antigen binding domain of a) comprises an antigen binding domain of a single domain antibody that specifically binds to CD47 such as for example human CD47.

In an exemplary embodiment, the antigen binding domain of e) comprises an antigen binding domain of a single domain antibody that specifically binds to human TROP2.

In an exemplary embodiment, the antigen binding domain of e) comprises an antigen binding domain of a single domain antibody that specifically binds to CD47, such as for example human CD47.

In some embodiments, the bispecific binding agent comprises two polypeptide chains. The two polypeptide chains are non-covalently assembled by amino acid residues of the CH2 and/or CH3 domain contained therein so as to obtain a bispecific and tetravalent binding agent.

In an exemplary embodiment, the binding agent or antigen binding domain(s) of the present disclosure may be capable of inhibiting the growth of tumor cells expressing human TROP2 and CD47.

In exemplary embodiments, the binding agent comprises one or more antigen binding domains that are capable of binding to CD47.

In another exemplary embodiments, the binding agent comprises one or more antigen binding domains that are capable of binding to TROP2 and one or more antigen binding domains that are capable of binding to CD47.

In an exemplary embodiment, the antigen binding domain that is capable of binding to CD47 comprises:
a. a CDRH1 having the amino acid sequence set forth in SEQ ID NO156, a CDRH2 having the amino acid sequence set forth in SEQ ID NO:157 and a CDRH3 having the amino acid sequence set forth in SEQ ID NO:158, or
b. a CDRH1 having the amino acid sequence set forth in SEQ ID NO159, a CDRH2 having the amino acid sequence set forth in SEQ ID NO:160 and a CDRH3 having the amino acid sequence set forth in SEQ ID NO:161.

In an exemplary embodiment, the antigen binding domain that is capable of binding to CD47 comprises an amino acid sequence as set forth in SEQ ID NO:155.

In an exemplary embodiment, the binding agent is capable of binding to CD47 and TROP2 and comprises an amino acid sequence as set forth in SEQ ID NO:155 and the amino acid sequence of at least one antigen binding domain of at least one single domain antibody disclosed herein.

In other exemplary embodiments, the binding agent may comprise at least one antigen binding domain capable of binding to CD47 and at least one antigen binding domain capable of binding to TROP2 and that comprises an amino acid sequence of an antigen binding domain of at least one single domain antibody set forth in any one of SEQ ID NOs:22-26, SEQ ID Nos:73-114. SEQ ID NOs:221-224, SEQ ID NO:231, SEQ ID NO:232, SEQ ID NO:239, SEQ ID NO:240, SEQ ID NO:247, SEQ ID NO:304, SEQ ID NO:295, SEQ ID NO:296, SEQ ID NO:297, SEQ ID NO:311, SEQ ID NO:318, SEQ ID NO:325, SEQ ID NO:332, SEQ ID NO:339, SEQ ID NO:346, SEQ ID NO:353, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503.

In other exemplary embodiments, the binding agent may comprise at least one antigen binding domain capable of binding to CD47 and at least one antigen binding domain capable of binding to TROP2 and that comprises an amino acid sequence of a humanized version of the single domain antibody disclosed herein.

In other exemplary embodiments, the binding agent may comprise at least one antigen binding domain capable of binding to PD-1 and at least one antigen binding domain capable of binding to TROP2.

In other exemplary embodiments, the binding agent may comprise at least one antigen binding domain capable of binding to TROP2 at least one antigen binding domain capable of binding to CD47 and at least one antigen binding domain capable of binding to PD-1.

In an exemplary embodiment, the antigen binding domain that is capable of binding to PD-1 comprises:
a. a CDRH1 having the amino acid sequence set forth in SEQ ID N0532, a CDRH2 having the amino acid sequence set forth in SEQ ID NO:535 and a CDRH3 having the amino acid sequence set forth in SEQ ID NO:534, or
b. a CDRH1 having the amino acid sequence set forth in SEQ ID NO:535, a CDRH2 having the amino acid sequence set forth in SEQ ID NO:536 and a CDRH3 having the amino acid sequence set forth in SEQ ID NO:537.

In an exemplary embodiment, the antigen binding domain that is capable of binding to PD-1 comprises an amino acid sequence as set forth in SEQ ID NO:538.

In an exemplary embodiment, the binding agent is capable of binding to PD-1 and TROP2 and comprises an amino acid sequence as set forth in SEQ ID NO:538 and the amino acid sequence of at least one antigen binding domain of at least one single domain antibody disclosed herein.

In some instances, the humanized version thereof has replacement of amino acid residues of their framework regions for those of a corresponding human or humanized framework regions.

In some instances, the humanized version has replacement of their framework region for those set forth in SEQ ID NO: 211. In other instances, the humanized version has replacement of their framework region for those set forth in SEQ ID NO: 213.

In some embodiment, the humanized version has replacement of their framework region for those set forth in any one of SEQ ID NOs: 248, 249, 250, 251, 252, 253, 254, 255, 256, 257, 258, 259, 260, 261, 262, 263, 264, 265, 266, 267, 268, 269, 270, or 271.

In some embodiment, the humanized version has replacement of their framework region for those set forth in any one of SEQ ID NOs: SEQ ID NOs: 272, 273, 274, 275, 276, 277, 278, 279, 280, 281, 282, 283, 284, 285, 286, 287, 288, 289, 290, 291, 292, 293 or 294.

In another exemplary embodiment, the binding agent is capable of binding to CD47 and TROP2 and comprises an amino acid sequence as set forth in SEQ ID NO:150 or in SEQ ID NO:151.

### Variants

Variants of the sequences disclosed herein are also encompassed by the present disclosure.

Binding agent of the present disclosure therefore encompass antibody variants (e.g., single domain antibody variants), variants of antibody-like molecule, variants of protein scaffold, variants of immune cell modulating agent. In addition, binding agents of the present disclosure may comprise an antigen binding fragment or an antigen binding domain of an antibody variant such as an antigen binding fragment or an antigen binding domain of a single domain antibody variant.

The present disclosure more particularly encompasses variants of the single domain antibody disclosed herein as well as binding agents that comprise one or more antigen binding domain of a single domain antibody variant. Variants of the binding agents disclosed herein may have the same format as the binding agent disclosed herein. As such, variants of the binding agents encompassed by the present disclosure may be multispecific and/or multivalent. Variants encompassed by the present disclosure include those which may comprise an insertion of one or more amino acid residues at one or more position, a deletion of one or more amino acid residues at one or more position or a substitution of one or more amino acid residues at one or more position (conservative or non-conservative substitutions).

The variation may occur in the antigen binding domain or in other elements of the binding agent such as the dimerization domain, linker or spacer.

For example, naturally occurring residues are divided into groups based on common side chain properties. Conservative substitutions may be made by exchanging an amino acid from one of the groups listed below (group 1 to 6) for another amino acid of the same group. Non-conservative substitutions will entail exchanging a member of one of these groups for another.
(group 1) hydrophobic: norleucine, methionine (Met), Alanine (Ala), Valine (Val), Leucine (Leu), Isoleucine (Ile)
(group 2) neutral hydrophilic: Cysteine (Cys), Serine (Ser), Threonine (Thr), Asparagine (Asn), Glutamine (Gln),
(group 3) acidic: Aspartic acid (Asp), Glutamic acid (Glu)
(group 4) basic: Histidine (His), Lysine (Lys), Arginine (Arg)
(group 5) residues that influence chain orientation: Glycine (Gly), Proline (Pro); and
(group 6) aromatic: Tryptophan (Trp), Tyrosine (Tyr), Phenylalanine (Phe)

Other exemplary embodiments of conservative substitutions are shown in Table 2 under the heading of “preferred substitutions”. If such substitutions result in an undesired property, then more substantial changes, denominated “exemplary substitutions” in Table 2, or as further described below in reference to amino acid classes, may be introduced and the products screened.

One of skill in the art will recognize that certain amino acids are less positively charged, are neutral, are negatively charged or have a reduced charge in comparison to other amino acids. Amino acids can be categorized based on net charge as indicated by an amino acid's isoelectric point. The isoelectric point is the pH at which the average net charge of the amino acid molecule is zero. When pH>pI, an amino acid has a net negative charge, and when the pH<pI, an amino acid has a net positive charge. In some embodiments, the measured pI value for an antibody is between about 3 and 9 (e.g. 3.0, 3.5, 4.0, 4.5, 5.0, 5.5, 6.0, 6.5, 7.0, 7.5, 8.0, 8.1, 8.2, 8.3, 8.4, 8.5, and 9) and any values in between. In some embodiments, the measured pI value for an antibody is between about 4 and 7 (e.g. 4.0, 4.5, 5.0, 5.5, 6.0, 6.5, 7.0), and any values in between. Exemplary isoelectric points of amino acids are shown in Table 2 below. Generally amino acids with positive electrically charged side chains include, for example, Arginine (R), Histidine (H), and Lysine (K). Amino acids with negative electrically charged side chains include, for example, Aspartic Acid (D) and Glutamic Acid (E). Amino acids with polar properties include, for example, Serine (5), Threonine (T), Asparagine (N), Glutamine (Q), and Cysteine (C), Tyrosine (Y) and Tryptophan (W). Non-polar amino acids include, for example, Alanine (A), Valine (V), Isoleucine (I), Leucine (L), Methionine (M), Phenylalanine (F), Glycine (G) and Proline (P).

In some embodiments, the isoelectric point of an antibody is modified via amino acid substitution. See, e.g. US20110076275. In some embodiments, modifying the isoelectric point of a polypeptide comprising an antibody results in a change in the antibody's half-life.

TABLE 2
Exemplary amino acid substitutions

| Original | | Conservative | pI (isoelectric |
|---|---|---|---|
| residue | Exemplary substitution | substitution | point) |
| Ala (A) | Val, Leu, Ile | Val | 6.0 |
| Arg (R) | Lys, Gln, Asn | Lys | 10.76 |
| Asn (N) | Gln, His, Lys, Arg, Asp | Gln | 5.41 |
| Asp (D) | Glu, Asn | Glu | 2.77 |
| Cys (C) | Ser, Ala | Ser | 5.07 |
| Gln (Q) | Asn; Glu | Asn | 5.65 |
| Glu (E) | Asp, Gln | Asp | 3.22 |
| Gly (G) | Ala | Ala | 5.97 |
| His (H) | Asn, Gln, Lys, Arg, | Arg | 7.59 |
| Ile (I) | Leu, Val, Met, Ala, Phe, | Leu | 6.02 |
| | norleucine | | |
| Leu (L) | Norleucine, Ile, Val, | Ile | 5.98 |
| | Met, Ala, Phe | | |
| Lys (K) | Arg, Gln, Asn | Arg | 9.74 |
| Met (M) | Leu, Phe, Ile | Leu | 5.74 |
| Phe (F) | Leu, Val, Ile, Ala, Tyr | Tyr | 5.48 |
| Pro (P) | Ala | Ala | 6.30 |
| Ser (S) | Thr | Thr | 5.58 |
| Thr (T) | Ser | Ser | 5.60 |
| Trp (W) | Tyr, Phe | Tyr | 5.89 |
| Tyr (Y) | Trp, Phe, Thr, Ser | Phe | 5.66 |
| Val (V) | Ile, Leu, Met, Phe, Ala, | Leu | 5.96 |
| | norleucine | | |

Generally, the degree of similarity and identity between variable chains is determined herein using the Blast2 sequence program (Tatiana A. Tatusova, Thomas L. Madden (1999), “Blast 2 sequences—a new tool for comparing protein and nucleotide sequences”, FEMS Microbiol Lett. 174:247-250) using default settings, i.e., blastp program, BLOSUM62 matrix (open gap 11 and extension gap penalty 1; gapx dropoff 50, expect 10.0, word size 3) and activated filters.

Percent identity will therefore be indicative of amino acids which are identical in comparison with the original peptide and which may occupy the same or similar position.

Percent similarity will be indicative of amino acids which are identical and those which are replaced with conservative amino acid substitution in comparison with the original peptide at the same or similar position.

Variants of the present disclosure may therefore comprise a sequence that is at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical with that of an original or reference sequence or a portion of an original sequence.

In an exemplary embodiment, mutations in one or more CDRs, variable region or constant region may be performed by amino acid substitution. Exemplary embodiments of substitutions include conservative amino acid substitutions or non-conservative amino acid substitutions. Exemplary substitutions are provided in Table 2. Other exemplary substitutions are provided in Table 13A. Yet other exemplary substitutions are provided in Table 13B.

In some embodiments, polypeptide chains having an amino acid sequence which is at least 75%, 80% 85%, 90%, 95%, 99% identical or less than 100% identical to a given amino acid sequence, may have amino acid substitutions, additions or deletions that are generally located outside of the complementarity determining regions.

In some embodiments, a variant may have at least 80% sequence identity with a sequence disclosed herein. In other embodiments, a variant may have at least 85% sequence identity with a sequence disclosed herein. In yet embodiments, a variant may have at least 90% sequence identity with a sequence disclosed herein. In further embodiments, a variant may have at least 95% sequence identity with a sequence disclosed herein. In other embodiments, a variant may have at least 99% sequence identity with a sequence disclosed herein.

In some embodiments, variation may occur in one or more CDRs. The mutations may occur for example, in CDR amino acid residues that are not involved in the interaction with TROP2. In an exemplary embodiment, amino acid residues of CDR1 and/or CDR2 may be mutated. In other exemplary embodiments, amino acid residues of CDR3 may be mutated. CDR mutations that improve affinity with TROP2 are particularly contemplated.

In other embodiments, variation may occur in the framework regions. The mutations may occur for example, in the framework amino acid residues that are not involved in the interaction with TROP2. In an exemplary embodiment, amino acid residues of FR1 and FR3 and/or FR4 may be mutated. In other exemplary embodiments, amino acid residues of FR2 may be mutated. FR2 mutations that improve affinity with TROP2 are particularly contemplated.

In other embodiments, the variations may occur in the constant region. In an exemplary embodiment, the constant region may be mutated so as to modulate ADCC activity. In another exemplary embodiment, the constant region may be mutated so as to modulate ADCP activity. In another exemplary embodiment, the constant region may be mutated so as to improve ADCP activity. In yet other exemplary embodiment, the constant region may be mutated so as to improve internalization. In yet other exemplary embodiment, the constant region may be mutated so as to improve stability.

Exemplary and non-limiting embodiments of mutations in the constant region (e.g., Fc region) that improves one or more effector functions are encompassed by the present disclosure, exemplary embodiments of which, are provided in the Table A (List of Mutations Antibodies (Basel). 2020 Nov. 17; 9(4):64, the entire content of which is incorporated herein by reference).

TABLE A

| | Effector | | | |
|---|---|---|---|---|
| | Function vs. WT | | | |
| Fc Modification(s) | ID | ADCC | ADCP | Reference |
| S298A/E333A/K334A | AAA | + | n.d. | R. J. Biol. Chem. 2001, 276, 6591- |
| | | | | 6604. |
| S239D/I332E | DE | ++ | + | Proc. Natl. Acad. Sci. USA 2006, |
| | | | | 103, 4005-401 |
| S239D/A330L/I332E | DLE | +++ | + | Proc. Natl. Acad. Sci. USA 2006, |
| | | | | 103, 4005-401 |
| G236A | G236A | − | + | Mol. Cancer Ther. 2008, 7, 2517- |
| | | | | 2527. |
| G236A/S239D/I332E | ADE | + | + | Mol. Cancer Ther. 2008, 7, 2517- |
| | | | | 2527. |
| F243L/R292P/Y300L/V305I/ | LPLIL | ++ | n.d. | Cancer Res. 2007, 67, 8882-8890. |
| P396L | | | | |
| L235V/F243L/R292P/Y300L/ | VLPLL | + | n.d. | Breast Cancer Res. 2011, 13, R123. |
| P396L | | | | |
| P247I/A339Q | | ++ | | Immunol. Cell Biol. 2020, 98, 287- |
| | | | | 304. |
| Afucosylation (Potelligent)* | Potelligent | ++ | n.d. | Biotechnol. Bioeng. 2004, 87, 614- |
| | | | | 622. |

*POTELLIGENT Technology involves the reduction of the amount of fucose in the carbohydrate structure of an antibody using a proprietary fucosyl transferase-knockout CHO cell line.
(−): no change;
(+): 2 fold increase;
(++): 2-9.99 fold increase;
(+++): 10-99.99;
(nd): no data.

Exemplary and non-limiting embodiments of single domain antibody variants are provided herein. Binding agents comprising one or more antigen binding domain(s) of one or more single domain antibody variants are also encompassed by the present disclosure.

Exemplary variants of the present disclosure may include polypeptides comprising the variable region sequence or heavy chain sequence of Table 1A, Table 1B, Table 15A to Table 15K, Table 16, Table 18 or Table 19 with one or more variations selected from amino acid substitution, deletion and/or addition. The variation may include for example from one to five amino acid substitutions, from one to five amino acid deletions and/or from one to five amino acid insertion. In an exemplary embodiment, the deletion or additions are consecutive. In another exemplary embodiment, the deletion or additions are non-consecutives. In yet another exemplary embodiment, the deletion or additions are at the N-terminus. In a further exemplary embodiment, the deletion or additions are at the C-terminus. In yet a further exemplary embodiment, the deletion or additions are outside of an IMGT CDR sequence. In yet a further exemplary embodiment, the deletion or additions are outside of an IMGT CDR3 sequence and/or IMGT FR2 sequence. In yet a further exemplary embodiment, the deletion or additions are outside of an Kabat CDR sequence. In yet a further exemplary embodiment, the deletion or additions are outside of a Kabat CDR3 sequence and/or Kabat FR2 sequence.

As used herein the term “from one to five” include any individual values such as “one”, “two”, “three”, “four” and “five” as well as any range comprised therebetween such as “from one to two”, “from one to three”, “from one to four”, “from one to five”, “from two to three”, “from two to four”, “from two to five”, “from three to four”, “from three to five” and “from four to five”.

Variants of the present disclosure may comprise at least one amino acid substitution in comparison with a sequence disclosed herein. Alternatively, variants of the present disclosure may comprise at least one amino acid deletion in comparison with a sequence disclosed herein. Alternatively, variants of the present disclosure may comprise at least one amino acid addition in comparison with a sequence disclosed herein.

In some embodiments, the variants of the present disclosure may comprise at least one amino acid substitution and at least one amino acid deletion in comparison with a sequence disclosed herein. In some embodiments, the variants of the present disclosure may comprise at least one amino acid substitution and at least one amino acid addition in comparison with a sequence disclosed herein. In some embodiments, the variants of the present disclosure may comprise at least one amino acid addition and at least one amino acid deletion in comparison with a sequence disclosed herein. In some embodiments, the variants of the present disclosure may comprise at least one amino acid substitution, at least one amino acid addition and at least one amino acid deletion in comparison with a sequence disclosed herein.

In some embodiments, the amino acid substitution, deletion or addition is outside of the amino acid sequence of CDR3 (IMGT or Kabat). In some embodiments, the amino acid substitution, deletion or addition is outside of the amino acid sequence of FR2 (IMGT or Kabat). In some embodiments, the amino acid substitution, deletion or addition is outside of the amino acid sequence of CDR1 (IMGT or Kabat). In some embodiments, the amino acid substitution, deletion or addition is outside of the amino acid sequence of CDR2 (IMGT or Kabat).

In an aspect of the present disclosure, single domain antibody variants include those having the amino acid sequence set forth in SEQ ID NO:143-146.

In accordance with the present disclosure, the single domain antibody or antigen binding fragment thereof comprises the amino acid sequence set forth in SEQ ID NO: 143.

SEQ ID NO: 143
(KD002 consensus)
X₁ₐVQLX_{1b}ESGGGX_{1c}VQX_{1d}GGSLRLSCAASGFPFSSADMSWVRQAPG
KX₁ₑX_{1f}EWVSX_{1g}INAX₁ₕGSKTYYX₁ᵢDX₁ⱼVKGRFTISRDNX₁ₖKNT
LYLX₁ₗMNX₁ₘLX₁ₙX₁ₒEDTAX₁ₚYX_{1q}CARAKLTDTHYVEDYWGQGT
X₁ᵣVTVSS.

In an exemplary embodiment, any of X₁ₐ to X₁ᵣ is each independently any (e.g., naturally occurring) amino acid residue.

In another exemplary embodiment, any of Xᵢₐ to X₁ᵣ is each independently an amino acid residue found at a corresponding position in any one of SEQ ID NO:23 or SEQ ID NOs:73-93 (SEQ ID NO:196), or conservative amino acid substitutions or non-conservative amino acid substitutions thereof.

In yet in another exemplary embodiment, any of X₁ₐ to X₁ᵣ is each independently an amino acid residue found at a corresponding position in any one of SEQ ID NO:22, SEQ ID NO:23, SEQ ID NO:24, SEQ ID NO:25 or SEQ ID NO:26 (SEQ ID NO:195) or conservative amino acid substitutions or non-conservative amino acid substitutions thereof.

In a further exemplary embodiment, any Xᵢₐ to X₁ᵣ is each independently an amino acid residue most frequently found at a corresponding position in any one of SEQ ID NO:23 or SEQ ID Nos:73-93.

In another exemplary embodiment, any Xᵢₐ to X₁ᵣ is each independently an amino acid residue most frequently found at a corresponding position in any one of SEQ ID NO:22, SEQ ID NO:23, SEQ ID NO:24, SEQ ID NO:25 to SEQ ID NO:26.

In yet another exemplary embodiment, any of X₁ₐ to X₁ᵣ is each independently an amino acid residue found at a corresponding position in any one of SEQ ID NOs: 73, 75, 76, 77, 78, 81, 82, 83, 85, 89 or 91 (SEQ ID NO:208), or conservative amino acid substitutions or non-conservative amino acid substitutions thereof.

In a further exemplary embodiment, any Xᵢₐ to X₁ᵣ is each independently an amino acid residue most frequently found at a corresponding position in any one of SEQ ID NOs: 73, 75, 76, 77, 78, 81, 82, 83, 85, 89 or 91.

In yet a further exemplary embodiment X₁ₐ is Q or E; X_{1b} is Q, V or L; X_{1c} is M or L; X₁ₐ is V or P; X₁ₑ is G or Q; X_{1f} is P, L or R; X_{1g} is Y or V; X₁ₕ is D, N, E; X₁ᵢ is P or A; X₁ⱼ is S or T; X₁ₖ is A or S; X₁ₗ is L or Q; X₁ₘ is N or S; X₁ₙ is K or R; X₁ₒ is P or A; X₁ₚ is L or V; X_{1q} is R or Y; and/or X₁ᵣ is Q or M (SEQ ID NO:196).

In another exemplary embodiment X₁ₐ is Q or E; X_{1b} is Q, V or L; X_{1c} is M or L; X₁ₐ is V or P; X₁ₑ is G or Q; X_{1f} is P, L or R; X_{1g} is Y; X₁ₕ is D, X₁ᵢ is P or A; X₁ⱼ is S or T; X₁ₖ is A or S; X₁ₗ is L or Q; X₁ₘ is N or S; X₁ₙ is K or R; X₁ₒ is P or A; X₁ₚ is L or V; X_{1q} is R or Y; and/or X₁ᵣ is Q or M (SEQ ID NO:197).

In accordance with the present disclosure, the single domain antibody or antigen binding fragment thereof comprises the amino acid sequence set forth in SEQ ID NO:211.

X₁ₐVQLX_{1b}ESGGGX_{1c}VQX_{1d}GGSLRLSCAAS[CDRH1]MSWVRQAPGK
X₁ₑX_{1f}EWVSX_{1g}[CDRH2]YYX₁ᵢDX₁ⱼVKGRFTISRDNX₁ₖKNTLYL
X₁ₗMNX₁ₘLX₁ₙX₁ₒEDTAX₁ₚYX_{1q}C[CDRH3]WGQGTX₁ᵣVTVSS

In some embodiments, any of X₁ₐ to X₁ᵣ is as described herein (e.g., SEQ ID NOs. 477, 478).

In other embodiments any of X₁ₐ to X₁ᵣ is a corresponding amino acid residue of an antibody heavy chain variable region.

In yet other embodiments any of X₁ₐ to X₁ᵣ is a corresponding amino acid residue of a sequence set forth in any one of SEQ ID NO:248-271.

In yet other embodiments any of X₁ₐ to X₁ᵣ is a corresponding amino acid residue of a human antibody heavy chain, such as, for example, a residue of a germline human antibody heavy chain.

In accordance with the present disclosure, [CDRH1], [CDRH2] and [CDRH3] in SEQ ID NO:211 correspond to the amino acid sequence of the IMGT CDR1, CDR2 and CDR3 of a given variable region sequence disclosed herein and in particular in Table 15A to Table 15K, Table 16, Table 18 or Table 19.

In accordance with the present disclosure, the single domain antibody or antigen binding fragment thereof comprises the amino acid sequence set forth in SEQ ID NO:144.

SEQ ID NO: 144
(KD005 consensus)
X₂ₐVQLX_{2b}ESGGGLX_{2c}X_{2d}X₂ₑGGSLRLSCAASGFTFSGSDMSWVRQA
PGKGX_{2f}EWVSX_{2g}ITSGGTTYYX₂ₕDX₂ᵢVKGRFTISRDNX₂ⱼKNTLYL
QMNSLX₂ₖX₂ₗX₂ₘDTAX₂ₙYX₂ₒCAKSRLTDSHYVEDAWGQGTX₂ₚVTV
SS

In an exemplary embodiment, any of X₂ₐ to X₂ₚ is each independently any (e.g., naturally occurring) amino acid residue.

In another exemplary embodiment, any of X₂ₐ to X₂ₚ is each independently an amino acid residue found at a corresponding position in any one of SEQ ID NO:26 or SEQ ID NO:94-114 (SEQ ID NO:199) or conservative amino acid substitutions or non-conservative amino acid substitutions thereof.

In another exemplary embodiment, any of X₂ₐ to X₂ₚ is each independently an amino acid residue found at a corresponding position in any one of SEQ ID NO:22, SEQ ID NO:23, SEQ ID NO:24, SEQ ID NO:25 or SEQ ID NO:26 (SEQ ID NO:198) or conservative amino acid substitutions or non-conservative amino acid substitutions thereof.

In yet another exemplary embodiment, any X₂ₐ to X₂ₚ is each independently an amino acid residue most frequently found at a corresponding position in any one of SEQ ID NO:26 or SEQ ID NO:94-114.

In a further exemplary embodiment, any X₂ₐ to X₂ₚ is each independently an amino acid residue most frequently found at a corresponding position in any one of SEQ ID NO:22, SEQ ID NO:23, SEQ ID NO:24, SEQ ID NO:25 to SEQ ID NO:26.

In yet a further exemplary embodiment, any of X₂ₐ to X₂ₚ is each independently an amino acid residue found at a corresponding position in any one of SEQ ID NOs: 94, 95, 96, 97, 98, 100, 101, 102, 103, 104, 105, 106, 107 or 108 (SEQ ID NO:209) or conservative amino acid substitutions or non-conservative amino acid substitutions thereof.

In an additional exemplary embodiment, any X₂ₐ to X₂ₚ is each independently an amino acid residue most frequently found at a corresponding position in any one of SEQ ID NOs: 94, 95, 96, 97, 98, 100, 101, 102, 103, 104, 105, 106, 107 or 108.

In another exemplary embodiment, any of X₂ₐ to X₂ₚ is each independently an amino acid residue found at a corresponding position in any one of SEQ ID NOs: 94, 95, 96, 97, 100, 102, 103, 104, 105, 106, 108, 112 or 114 (SEQ ID NO:210) or conservative amino acid substitutions or non-conservative amino acid substitutions thereof.

In a further exemplary embodiment, any X₂ₐ to X₂ₚ is each independently an amino acid residue most frequently found at a corresponding position in any one of SEQ ID NOs: 94, 95, 96, 97, 100, 102, 103, 104, 105, 106, 108, 112 or 114.

In yet a further exemplary embodiment, any of X₂ₐ to X₂ₚ is each independently an amino acid residue found at a corresponding position in any one of SEQ ID NOs: 94, 95, 96, 97, 98, 99, 100, 101, 102, 104, 105, 112, 113 or 114 (SEQ ID NO:210) or conservative amino acid substitutions or non-conservative amino acid substitutions thereof.

In another exemplary embodiment, any X₂ₐ to X₂ₚ is each independently an amino acid residue most frequently found at a corresponding position in any one of SEQ ID NOs: 94, 95, 96, 97, 98, 99, 100, 101, 102, 104, 105, 112, 113 or 114.

In another exemplary embodiment, any of X₂ₐ to X₂ₚ is each independently an amino acid residue found at a corresponding position in any one of SEQ ID NOs: 94, 95, 96, 97, 102 or 105 (SEQ ID NO:210) or conservative amino acid substitutions or non-conservative amino acid substitutions thereof.

In an additional exemplary embodiment, any X₂ₐ to X₂ₚ is each independently an amino acid residue most frequently found at a corresponding position in any one of SEQ ID NOs: 94, 95, 96, 97, 102 or 105.

In another exemplary embodiment, X₂ₐ is Q or E; X_{2b} is Q, L or V; X_{2c} is V or I; X_{2d} is E or Q; X₂ₑ is A or P; X_{2f} is P, or L; X_{2g} is Y, A or V; X₂ₕ is P or A; X₂ᵢ is S or T; X₂ⱼ is A or S; X₂ₖ is K or R; X₂ₗ is P or A; X₂ₘ is D or E; X₂ₙ is L or V; X₂ₒ is R or Y and/or X₂ₚ is Q or L (SEQ ID NO:199).

In yet another exemplary embodiment, X₂ₐ is Q or E; X_{2b} is Q or L; X_{2c} is V or I; X_{2d} is E or Q; X₂ₑ is A or P; X_{2f} is P, or L; X_{2g} is Y or A; X₂ₕ is P or A; X₂ᵢ is S or T; X₂ⱼ is A or S; X₂ₖ is K or R; X₂ₗ is P or A; X₂ₘ is D or E; X₂ₙ is L or V; X₂ₒ is R or Y and/or X₂ₚ is Q or L (SEQ ID NO:200).

In accordance with the present disclosure, the single domain antibody or antigen binding fragment thereof comprises the amino acid sequence set forth in SEQ ID NO:213.

X₂ₐVQLX_{2b}ESGGGX_{2q}X_{2c}X_{2d}X₂ₑGGSLRLSCX₂ᵣAS[CDRH1]MSWV
RQAPGKGX_{2f}EWVSX_{2g}[CDRH2]YYX₂ₕDX₂ᵢVKGRFTISRDNX₂ⱼKNT
LYLQMNSLX₂ₖX₂ₗX₂ₘDTAX₂ₙYX₂ₒC[CDRH3]X₂ₛGQGTX₂ₚVTVSS

In some embodiments, any of X₂ₐ to X₂ₚ is as described herein and X_{2g} is L, X₂ᵣ is A and/or X₂ₛ is W.

In other embodiments any of X₂ₐ to X₂ₛ is a corresponding amino acid residue of an antibody heavy chain variable region.

In yet other embodiments any of X₂ₐ to X₂ₛ is a corresponding amino acid residue of a sequence set forth in any one of SEQ ID NO:272-294.

In yet other embodiments any of X₂ₐ to X₂ₛ is a corresponding amino acid residue of a human antibody heavy chain, such as, for example, a residue of a germline human antibody heavy chain.

In some embodiments, X₂ₐ is Q or E; X_{2b} is Q, L or V; X_{2c} is V or I; X_{2d} is E, Q, D or H; X₂ₑ is A or P; X_{2f} is P, or L; X_{2g} is Y, A or V; X₂ₕ is P or A; X₂ᵢ is S or T; X₂ⱼ is A or S; X₂ₖ is K, R or T; X₂ₗ is P or A; X₂ₘ is D or E; X₂ₙ is L or V; X₂ₒ is R or Y; X₂ₚ is Q or L; X_{2q} is L; X₂ᵣ is A and/or X₂ₛ is W (SEQ ID NO:479).

In some embodiments, X₂ₐ is Q or E; X_{2b} is Q, L or V; X_{2c} is V or I; X_{2d} is E, Q, D, or H; X₂ₑ is A or P; X_{2f} is P, or L; X_{2g} is Y, A, V or I; X₂ₕ is P or A; X₂ᵢ is S or T; X₂ⱼ is A or S; X₂ₖ is K, R or T; X₂ₗ is P or A; X₂ₘ is D or E; X₂ₙ is L or V; X₂ₒ is R or Y; X₂ₚ is Q or L; X_{2q} is L or M; X₂ᵣ is A or V and/or X₂ₛ is W or Q (SEQ ID NO:480).

In accordance with the present disclosure, [CDRH1], [CDRH2] and [CDRH3] in SEQ ID NO:213 correspond to the amino acid sequence of the IMGT CDR1, CDR2 and CDR3 of a given variable region sequence disclosed herein and in particular in Table 15A to Table 15K, Table 16, Table 18 or Table 19.

In accordance with the present disclosure, the single domain antibody or antigen binding fragment thereof comprises the amino acid sequence set forth in SEQ ID NO:145.

SEQ ID NO: 145 (Consensus from KD001 to KD005 heavy chain)

QVQLQESGGGX₃ₐVX_{3b}X_{3c}GX_{3d}SLRLSCAASGFX₃ₑFSX_{3f}X_{3g}DMSW
VRQAPGKGPEWVSYIX₃ₕX₃ᵢX₃ⱼX₃ₖX₃ₗX₃ₘTX₃ₙYPDSVKGRFTX₃ₒS
RDNAKNX₃ₚLYLX_{3q}MNX₃ᵣLX₃ₛPX₃ₜDTALYRCAX₃ᵤX₃ᵥX_{3w}LTDX₃ₓH
YVEDX_{3y}WGQGTQVTVSS

In an exemplary embodiment any of X₃ₐ to X_{3y} is each independently any (e.g., naturally occurring) amino acid residue.

In another exemplary embodiment, any of X₃ₐ to X_{3y} is each independently an amino acid residue found at a corresponding position in any one of SEQ ID NO:22, SEQ ID NO:23, SEQ ID NO:24, SEQ ID NO:25 or SEQ ID NO:26 (SEQ ID NO:201) or conservative amino acid substitutions or non-conservative amino acid substitutions thereof.

In yet another exemplary embodiment, any of X₃ₐ to X_{3y} is each independently an amino acid residue most frequently found in the group consisting of SEQ ID NO:22, SEQ ID NO:23, SEQ ID NO:24, SEQ ID NO:25 to SEQ ID NO:26.

In a further exemplary embodiment, X₃ₐ is M or L; X_{3b} is Q, H or E; X_{3c} is V, P or A; X_{3d} is G or R; X₃ₑ is P or T; X_{3f} is S, N or G; X_{3g} is A, Y or S; X₃ₕ is N or T; X₃ᵢ is A, S or G; X₃ⱼ is D, G or S; X₃ₖ is G or absent; X₃ₗ is S or G; X₃ₘ is K, N or T; X₃ₙ is Y or D; X₃ₒ is I or T; X₃ₚ is T or M; X₃₉ is L or Q; X₃ᵣ is N or S; X₃ₛ is K or T; X₃ₜ is E or D; X₃ᵤ is R or K; X₃ᵥ is A or S; X_{3w} is K or R; X₃ₓ is T or S; and/or X_{3y} is Y or A (SEQ ID NO:202).

In yet a further exemplary embodiment, X₃ₐ is M or L; X_{3b} is Q, H or E; X_{3c} is V, P or A; X_{3d} is G or R; X₃ₑ is P or T; X_{3f} is S, N or G; X_{3g} is A, Y or S; X₃ₕ is N or T; X₃ᵢ is A, S or G; X₃ⱼ is D, G or S; X₃ₖ is G or absent; X₃ₗ is S or G; X₃ₘ is K, N or T; X₃ₙ is Y; X₃ₒ is I or T; X₃ₚ is T or M; X_{3q} is L or Q; X₃ᵣ is N or S; X₃ₛ is K or T; X₃ₜ is E or D; X₃ᵤ is R or K; X₃ᵥ is A or S; X_{3w} is R; X₃ₓ is S; and/or X_{3y} is A (SEQ ID NO:203).

Generally, the position of amino acid residues is as set forth for SEQ ID NO:145 and as exemplified in Table 1C.

SEQ ID NO: 145 amino acid residues numbering (IMGT CDR position bold underlined).
Exemplary interacting residues are indicated in each third row.

| 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|
| Q | V | Q | L | Q | E | S | G | G | G | X₃ₐ | V | X_{3b} | X_{3c} | G | X_{3d} |
| Q | V | Q | L | | | | | | | | | | | | |

| 17 | 18 | 19 | 20 | 21 | 22 | 23 | 24 | 25 | 26 | 27 | 28 | 29 | 30 | 31 |
|---|---|---|---|---|---|---|---|---|---|---|---|---|---|---|
| S | L | R | L | S | C | A | A | S | G | F | X₃ₑ | F | S | X_{3f} |

| 32 | 33 | 34 | 35 | 36 | 37 | 38 | 39 | 40 | 41 | 42 | 43 | 44 | 45 | 46 |
|---|---|---|---|---|---|---|---|---|---|---|---|---|---|---|
| X_{3g} | D | M | S | W | V | R | Q | A | P | G | K | G | P | E |
| | | | | | V | | Q | | | | | | P | E |

| 47 | 48 | 49 | 50 | 51 | 52 | 53 | 54 | 54a | 55 | 56 | 57 | 58 | 59 | 60 |
|---|---|---|---|---|---|---|---|---|---|---|---|---|---|---|
| W | V | S | Y | I | X₃ₕ | X₃ᵢ | X₃ⱼ | X₃ₖ | X₃ₗ | X₃ₘ | T | X₃ₙ | Y | P |
| W | | | Y | | | | | | | | | Y | | |

| 61 | 62 | 63 | 64 | 65 | 66 | 67 | 68 | 69 | 70 | 71 | 72 | 73 | 74 | 75 |
|---|---|---|---|---|---|---|---|---|---|---|---|---|---|---|
| D | S | V | K | G | R | F | T | X₃ₒ | S | R | D | N | A | K |

| 76 | 77 | 78 | 79 | 80 | 81 | 82 | 83 | 84 | 85 | 86 | 87 | 88 | 89 | 90 |
|---|---|---|---|---|---|---|---|---|---|---|---|---|---|---|
| N | X₃ₚ | L | Y | L | X_{3q} | M | N | X₃ᵣ | L | X₃ₛ | P | X₃ₜ | D | T |

| 91 | 92 | 93 | 94 | 95 | 96 | 97 | 98 | 99 | 100 | 101 | 102 | 103 | 104 | 105 |
|---|---|---|---|---|---|---|---|---|---|---|---|---|---|---|
| A | L | Y | R | C | A | X₃ᵤ | X₃ᵥ | X_{3w} | L | T | D | X₃ₓ | H | Y |
| | | | | | | | | R | L | T | D | S | H | Y |

| 106 | 107 | 108 | 109 | 110 | 111 | 112 | 113 | 114 | 115 | 116 | 117 | 118 | 119 | 120 |
|---|---|---|---|---|---|---|---|---|---|---|---|---|---|---|
| V | E | D | X_{3y} | W | G | Q | G | T | Q | V | T | V | S | S |
| V | E | D | A | W | G | Q | | | | | | | | |

In accordance with the present disclosure, the single domain antibody or antigen binding fragment thereof comprises the amino acid sequence set forth in SEQ ID NO: 146.

SEQ ID NO: 146
(Consensus for KD001-KD005 and selected variants
heavy chains)
X₄ₐVQLX_{4b}ESGGGX_{4c}X_{4d}X₄ₑX_{4f}GX_{4g}SLRLSCAASGFX₄ₕFSX₄ᵢ
X₄ⱼDMX₄ₖWX₄ₗRQAPGKX₄ₘX₄ₙX₄ₒWVSX₄ₚIX_{4q}X₄ᵣX₄ₛGX₄ₜ
X₄ᵤTX₄ᵥYX_{4w}DX₄ₓVKGRFTX_{4y}SRDNX_{4z}KNX₅ₐLYLX_{5b}MNX_{5c}
X_{5d}X₅ₑX_{5f}DTAX_{5g}YX₅ₕCAX₅ᵢX₅ⱼX₅ₖLTDX₅ₗHYVEDX₅ₘWGQGT
X₅ₙVTVSS.

In an exemplary embodiment, any of X₄ₐ to X₅₁ is each independently any (e.g., naturally occurring) amino acid residue.

In another exemplary embodiment, any of X₄ₐ to X₅₁ is each independently an amino acid residue found at a corresponding position in any one of SEQ ID NOs:22-26, SEQ ID Nos:73-114 (SEQ ID NO:205) or conservative amino acid substitutions or non-conservative amino acid substitutions thereof.

In yet another exemplary embodiment, any of X₄ₐ to X₅₁ is each independently an amino acid residue found at a corresponding position in any one of SEQ ID NO:22, SEQ ID NO:23, SEQ ID NO:24, SEQ ID NO:25 or SEQ ID NO:26 (SEQ ID NO:204) or conservative amino acid substitutions or non-conservative amino acid substitutions thereof.

In another exemplary embodiment, any of X₄ₐ to X₅ₙ is each independently an amino acid residue most frequently found at a corresponding position in any one of SEQ ID NOs:22-26, SEQ ID Nos:73-114.

In another exemplary embodiment, any of X₄ₐ to X₅₁ is each independently an amino acid residue most frequently found at a corresponding position in any one of SEQ ID NO:22, SEQ ID NO:23, SEQ ID NO:24, SEQ ID NO:25 to SEQ ID NO:26.

In a further exemplary embodiment, X₄ₐ is Q or E, X_{4b} is Q, L or V, X_{4c} is M or L, X₄ₐ is V or I, X₄ₑ is Q, H or E, X_{4f} is P, A or V, X_{4g} is G or R, X₄ₕ is P or T, X₄ᵢ is G, S or N, X₄ⱼ is A or S, X₄ₖ is S or H, X₄ₗ is V or Y, X₄ₘ is G or Q, X₄ₙ is P, L or R, X₄ₒ is E or V, X₄ₚ is Y, A or R, X_{4q} is T or N, X₄ᵣ is A, S or G, X₄ₛ is G, S, D, N or E, X₄ₜ is absent or S, X₄ᵤ is K or T, X₄ᵥ is Y, D or S, X_{4w} is P or A, X₄ₓ is S or T, X_{4y} is I or T, X_{4z} is S or A X₅ₐ is T or M X_{5b} is Q or L, X_{5c} is S or N, X_{5d} is K, R or T, X₅ₑ is P or A, X_{5f} is E or D, X_{5g} is V or L, X₅ₕ is Y or R, X₅ᵢ is R or K, X₅ⱼ is A or S, X₅ₖ is K or R, X₅ₗ is S or T, X₅ₘ is Y or A, and/or X₅ₙ is Q, L or M (SEQ ID NO:205).

The antibodies of Table 15B and Table 15C all have CDR1, CDR2 and CDR3 identical to KD001 (KD008) CDRs.

In an aspect of the present disclosure, single domain antibody variants include those having the amino acid sequence set forth in SEQ ID NO:507, SEQ ID NO:508, SEQ ID NO:509 (e.g., Table 15B and 15C).

SEQ ID NO: 507, 508, 509 (Consensus for KD008 and
KD008 variants)
XVQLXESGGGXXXXGXSLRLSCXASGFTFSGSDMSWVRQAPGKGXEWVSY
ITSGGTTYYXDXVKGRFTISRDNXXNXLYLQMNSLXXXDTAXXRCAKARL
TDSHYVEDAXGQGTXVTVSS

In an exemplary embodiment, X from position 1 to 115 in SEQ ID NO:507 is each independently any (e.g., naturally occurring) amino acid residue.

In other exemplary embodiments, X at position 1 is E, Q, N, K or D; X at position 5 is any amino acid residue; X at position 11 is L, M, I or V; X at position 12 is I, V, L or M; X at position 13 is Q, H or D; X at position 14 is any amino acid residue; X at position 16 is G, D, S or N; X at position 23 is A, V or T; X at position 45 is any amino acid residue; X at position 60 is any amino acid residue; X at position 62 is S, T or A; X at position 74 is A, S or T; X at position 75 is K, N, E, Q, D or H; X at position 77 is any amino acid residue; X at position 86 is any amino acid residue; X at position 87 is any amino acid residue; X at position 88 is D, E, N or Q; X at position 92 is L, V, M or I; X at position 93 is any amino acid residue; X at position 110 is any amino acid residue and/or X at position 115 is any amino acid residue (SEQ ID NO:508).

In some embodiments, X at position 1 is E or Q; X at position 5 is Q or L; X at position 11 is L or M; X at position 12 is I or V; X at position 13 is Q, H or D; X at position 14 is P or A; X at position 16 is G or D; X at position 23 is A or V; X at position 45 is P or L; X at position 60 is P or A; X at position 62 is S or T; X at position 74 is A or S; X at position 75 is K or N; X at position 77 is T or M; X at position 86 is R, K or T; X at position 87 is P or A; X at position 88 is D or E; X at position 92 is L or V; X at position 93 is Y or D; X at position 110 is W or Q and/or X at position 115 is L or Q (SEQ ID NO:509).

In some embodiments, X at position 1 is E or Q; X at position 5 is Q or L; X at position 11 is L or M; X at position 12 is I or V; X at position 13 is Q, H or D; X at position 14 is P or A; X at position 16 is D; X at position 23 is A or V; X at position 45 is P or L; X at position 60 is P or A; X at position 62 is S or T; X at position 74 is A or S; X at position 75 is N; X at position 77 is M; X at position 86 is R, K or T; X at position 87 is P or A; X at position 88 is D or E; X at position 92 is L or V; X at position 93 is D; X at position 110 is W or Q and/or X at position 115 is L or Q (SEQ ID NO:509).

In some embodiments X at position 11 is M or L; X at position 13 is H, D or Q; X at position 14 is P or A; X at position 16 is G or D; X at position 23 is A or V; X at position 75 is K or N; X at position 77 is T or M; X at position 86 is K or T; X at position 88 is D or E; X at position 93 is Y or D and/or X at position 110 is W or Q (SEQ ID NO:510).

In some embodiments X at position 1 is E or Q; X at position 5 is Q or L; X at position 11 is L or M; X at position 12 is I or V; X at position 13 is Q or H; X at position 45 is P or L; X at position 60 is P or A; X at position 62 is S or T; X at position 74 is A or S; X at position 86 is R or T; X at position 87 is A or P; X at position 88 is D or E; X at position 92 is L or V and/or X at position 115 is L or Q (SE ID NO:511).

In other exemplary embodiments, any one of X from position 1 to 115 in SEQ ID NO:507 is each independently an amino acid residue found at a corresponding position in any one of SEQ ID NO:221, SEQ ID NO:222, SEQ ID NO:223, SEQ ID NO:224, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:469, SEQ ID NO:470, SEQ ID NO:471, SEQ ID NO:472 or SEQ ID NO:473 or conservative amino acid substitutions or non-conservative amino acid substitutions thereof.

In yet other exemplary embodiments, any one of X from position 1 to 115 in SEQ ID NO:507 is each independently an amino acid residue most frequently found at a corresponding position in any one of SEQ ID NO:221, SEQ ID NO:222, SEQ ID NO:223, SEQ ID NO:224, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:469, SEQ ID NO:470, SEQ ID NO:471, SEQ ID NO:472 or SEQ ID NO:473.

In an aspect of the present disclosure, single domain antibody variants include those having the amino acid sequence set forth in SEQ ID NO:512 or SEQ ID NO:513 (e.g., Table 15D).

QVQLQESGGGLVQPGXSLRLSCAASGFTFSGSDMSWVRQAPGKGPEWVSY
ITGSGTTYYPDSVKGRFTXSRDNAKNXLYLQMNSLKPDDTALYRCAKARL
TDSHYVEDAWGQGTQVTVSS

In an exemplary embodiment, X from position 16, 69 or 77 in SEQ ID NO:512 is each independently any (e.g., naturally occurring) amino acid residue.

In some embodiments X at position 16 is R or G; X at position 69 is T or I and/or X at position 77 is M or T (SEQ ID NO:513).

In yet other embodiments, any of X at position 16 or 69 in SEQ ID NO:512 is a conservative amino acid substitution or a non-conservative amino acid substitution of the amino acid residue found at the corresponding position in SEQ ID NO:25 or SEQ ID NO:304.

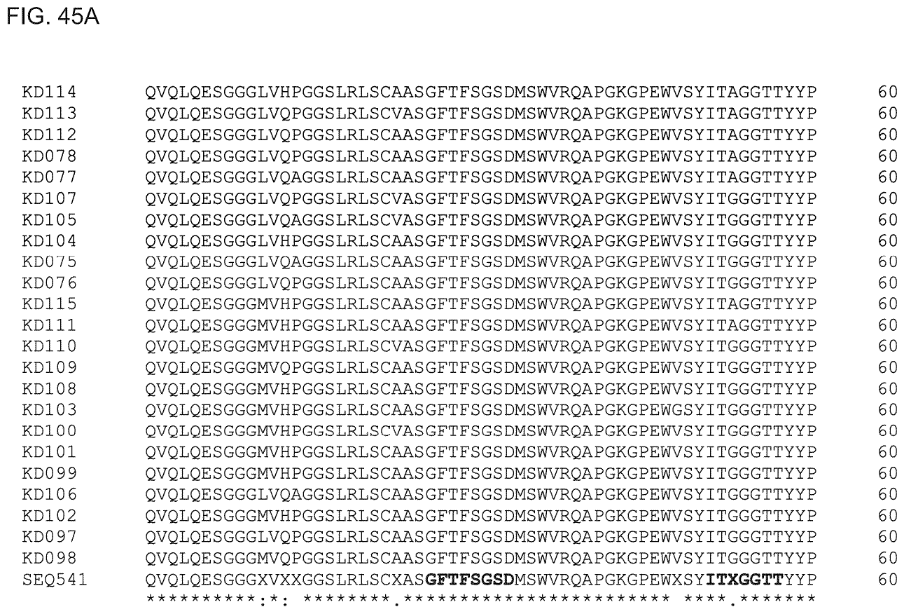

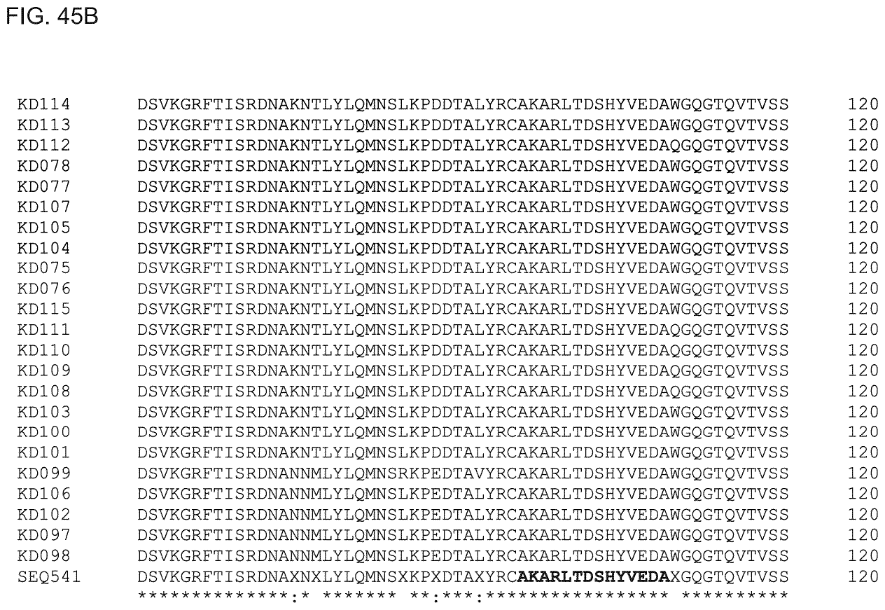

The antibodies of Table 15E, Table 15F, Table 15G, Table 15H and Table 151 all have same CDR1 and CDR3 and variation within CDR2 (FIGS. 45A-45B).

In an aspect of the present disclosure, single domain antibody variants include those having the amino acid sequence set forth in SEQ ID NO:539, SEQ ID NO:540 or SEQ ID NO:541 (e.g., Table 15E-F).

(SEQ ID NO: 541)
QVQLQESGGGXVXXGGSLRLSCXASGFTFSGSDMSWVRQAPGKGPEWXSY
ITXGGTTYYPDSVKGRFTISRDNAXNXLYLQMNSXKPXDTAXYRCAKARL
TDSHYVEDAXGQGTQVTVSS

In an exemplary embodiment, X at position 11, 13, 14, 23, 48, 75, 77, 85, 88, 92 or 110 is any amino acid residue and X at position 53 is A or G (SEQ ID NO:539).

In yet other embodiments, any of X at position 11, 13, 14, 23, 48, 75, 77, 85, 88, 92 or 110 is a conservative amino acid substitution or a non-conservative amino acid substitution of the amino acid residue found at the corresponding position in any one of SEQ ID NO:231, SEQ ID NO:232, SEQ ID NO:295, SEQ ID NO:296, SEQ ID NO:297, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:239, SEQ ID NO:240, SEQ ID NO:383, SEQ ID NO:384, SEQ ID NO:385, SEQ ID NO:386, or SEQ ID NO:387 and X at position 53 is A or G (SEQ ID NO:539).

In some embodiments X at position 11 is M, L, I or V; X at position 13 is H, Q, N, K or R; X at position 14 is any amino acid residues; X at position 23 is A, V or T; X at position 48 is any amino acid residues; X at position 53 is A or G X at position 75 is K, N, E, Q or H; X at position 77 is any amino acid residues; X at position 85 is any amino acid residues; X at position 88 is E, D, N or Q; X at position 92 is L, V, M or I; and/or X at position 110 is any amino acid residues.

In other embodiments X at position 11 is M or L; X at position 13 is H or Q; X at position 14 is P or A; X at position 23 is A or V; X at position 48 is V or G; X at position 53 is A or G X at position 75 is K or N; X at position 77 is T or M; X at position 85 is R or L; X at position 88 is E or D; X at position 92 is L or V; and/or X at position 110 is W or Q.

In an aspect of the present disclosure, single domain antibody variants include those having the amino acid sequence set forth in SEQ ID NO:514, SEQ ID NO:515 or SEQ ID NO:516 (e.g., Table 15E).

QVQLQESGGGXVXXGGSLRLSCXASGFTFSGSDMSWVRQAPGKGPEWXSY
ITGGGTTYYPDSVKGRFTISRDNAXNXLYLQMNSXKPXDTAXYRCAKARL
TDSHYVEDAXGQGTQVTVSS

In an exemplary embodiment, X from position 11 to 110 in SEQ ID NO:514 is each independently any (e.g., naturally occurring) amino acid residue.

In some embodiments, X at position 11 is M, L, I or V; X at position 13 is H, Q, N, K or R; X at position 14 is any amino acid residues; X at position 23 is A, V or T; X at position 48 is any amino acid residues; X at position 75 is K, N, E, Q or H; X at position 77 is any amino acid residues; X at position 85 is any amino acid residues; X at position 88 is E, D, N or Q; X at position 92 is L, V, M or I and/or X at position 110 is any amino acid residues (SEQ ID NO:515).

In some embodiments, X at position 11 is M or L; X at position 13 is H or Q; X at position 14 is P or A; X at position 23 is A or V; X at position 48 is V or G; X at position 75 is K or N; X at position 77 is T or M; X at position 85 is R or L; X at position 88 is E or D; X at position 92 is L or V and/or X at position 110 is W or Q (SEQ ID NO:516).

In other exemplary embodiments, any one of X from position 11 to 110 in SEQ ID NO:514 is each independently an amino acid residue found at a corresponding position in any one of SEQ ID NO:231, SEQ ID NO:232, SEQ ID NO:295, SEQ ID NO:296, SEQ ID NO:297, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, or SEQ ID NO:382 or conservative amino acid substitutions or non-conservative amino acid substitutions thereof.

In yet other exemplary embodiments, any one of X from position 11 to 110 in SEQ ID NO:514 is each independently an amino acid residue most frequently found at a corresponding position in any one of SEQ ID NO:231, SEQ ID NO:232, SEQ ID NO:295, SEQ ID NO:296, SEQ ID NO:297, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, or SEQ ID NO:382.

In an aspect of the present disclosure, single domain antibody variants include those having the amino acid sequence set forth in SEQ ID NO:517, SEQ ID NO:518 or SEQ ID NO:519 (e.g., Table 15F).

QVQLQESGGGXVXXGGSLRLSCXASGFTFSGSDMSWVRQAPGKGPEWVSY
ITAGGTTYYPDSVKGRFTISRDNAKNTLYLQMNSLKPDDTALYRCAKARL
TDSHYVEDAXGQGTQVTVSS

In an exemplary embodiment, X from position 11 to 110 in SEQ ID NO:517 is each independently any (e.g., naturally occurring) amino acid residue.

In some embodiments X at position 11 is L, M, I or V; X at position 13 is Q, H, N or K; X at position 14 is any amino acid; X at position 23 is A, V or T and/or X at position 110 is any amino acid (SEQ ID NO:518).

In some embodiments X at position 11 is L or M; X at position 13 is Q or H; X at position 14 is A or P; X at position 23 is A or V and/or X at position 110 is W or Q (SEQ ID NO:519).

In other exemplary embodiments, any one of X from position 11 to 110 in SEQ ID NO:517 is each independently an amino acid residue found at a corresponding position in any one of SEQ ID NO:239, SEQ ID NO:240, SEQ ID NO:383, SEQ ID NO:384, SEQ ID NO:385, SEQ ID NO:386 or SEQ ID NO:387 or conservative amino acid substitutions or non-conservative amino acid substitutions thereof.

In yet other exemplary embodiments, any one of X from position 11 to 110 in SEQ ID NO:517 is each independently an amino acid residue most frequently found at a corresponding position in any one of SEQ ID NO:239, SEQ ID NO:240, SEQ ID NO:383, SEQ ID NO:384, SEQ ID NO:385, SEQ ID NO:386 or SEQ ID NO:387.

In an aspect of the present disclosure, single domain antibody variants include those having the amino acid sequence set forth in SEQ ID NO:520, SEQ ID NO:521 or SEQ ID NO:522 (e.g., Table 15G).

QVQLQESGGGXVXPGGSLRLSCAASGFTFSGSDMSWVRQAPGKGPEWVSY
ITSGGGTYYPDSVKGRFTISRDNAKNTLYLQMNSLKPEDTALYRCAKARL
TDSHYVEDAWGQGTQVTVSS

In an exemplary embodiment, X at position 11 and 14 in SEQ ID NO:520 is each independently any (e.g., naturally occurring) amino acid residue.

In some embodiments, X at position 11 is M, L, I or V and/or X at position 14 is H, Q, N or K (SEQ ID NO:521).

In some embodiments X at position 11 is M or L and/or X at position 14 is H or Q (SEQ ID NO:522).

In other exemplary embodiments, any one of X at position 11 and 14 of SEQ ID NO:520 is a conservative amino acid substitution or a non-conservative amino acid substitution of the amino acid residue found at the corresponding position in SEQ ID NO:247 or SEQ ID NO:388.

In an aspect of the present disclosure, single domain antibody variants include those having the amino acid sequence set forth in SEQ ID NO:523, SEQ ID NO:524 or SEQ ID NO:525 (e.g., Table 15H).

QVQLQESGGGMVHPGGSLRLSCAASGFTFSGSDMSWVRQAPGKGPEWVSY
ITGGGGTYYADSVKGRFTISRDXAKNXLYLQMNSLKPEDTALYRCAKARL
TDSHYVEDAWGQGTQVTVSS

In an exemplary embodiment, X at position 73 and 77 in SEQ ID NO:523 is each independently any (e.g., naturally occurring) amino acid residue.

In some embodiments, X at position 73 is N, H, Q or K and/or X at position 77 is any amino acid (SEQ ID NO:524).

In some embodiments X at position 73 is N or H and/or X at position 77 is M or T (SEQ ID NO:525).

In other exemplary embodiments, any one of X at position 73 and 77 of SEQ ID NO:523 is a conservative amino acid substitution or a non-conservative amino acid substitution of the amino acid residue found at the corresponding position in SEQ ID NO:325 or SEQ ID NO:390.

In an aspect of the present disclosure, single domain antibody variants include those having the amino acid sequence set forth in SEQ ID NO:526, SEQ ID NO:527 or SEQ ID NO:528 (e.g., Table 15I).

QVQLQESGGGXVXPGGSLRLSCAASGFTFSGSDMSWVRQAPGKGPEWVSY
ITAGGSTEYADSVKGRFTISRDNAKKKLYLQMNSLKPEDTALYRCAKARL
TDSHYVEDAWGQGTQVTVSS

In an exemplary embodiment, X at position 11 and 13 in SEQ ID NO:526 is each independently any (e.g., naturally occurring) amino acid residue.

In some embodiments X at position 11 is M, L, I or V and/or X at position 13 is any amino acid residue (SEQ ID NO:527).

In some embodiments X at position 11 is M or L and/or X at position 13 is Q or L (SEQ ID NO:528).

In other exemplary embodiments, any one of X at position 11 and 13 of SEQ ID NO:526 is a conservative amino acid substitution or a non-conservative amino acid substitution of the amino acid residue found at the corresponding position in SEQ ID NO:397 or SEQ ID NO:398.

In an aspect of the present disclosure, single domain antibody variants include those having the amino acid sequence set forth in SEQ ID NO:529, SEQ ID NO:530 or SEQ ID NO:531 (e.g., Table 15J).

In an exemplary embodiment, X from position 11 to 89 in SEQ ID NO:529 is each independently any (e.g., naturally occurring) amino acid residue.

In some embodiments, X at position 11 is M, L, I or V; X at position 76 is N, K, E, Q or D; X at position 78 is any amino acid residues and/or X at position 89 is D, E, N or Q (SEQ ID NO:530).

In some embodiments, X at position 11 is M or L; X at position 76 is N or K; X at position 78 is M or T and/or X at position 89 is D or E (SEQ ID NO:531).

In other exemplary embodiments, any one of X from position 11 to 89 in SEQ ID NO:529 is a conservative amino acid substitution or a non-conservative amino acid substitution of the amino acid residue found at the corresponding position in SEQ ID NO:311 or SEQ ID NO:389.

In an exemplary embodiment, the binding agent or antigen binding domain(s) thereof may comprise the amino acid sequence of the CDR3 set forth in SEQ ID NO:126, SEQ ID NO:185 or SEQ ID NO:186.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR3 is as set forth in SEQ ID NO:126, SEQ ID NO:185 or SEQ ID NO:186.

In an exemplary embodiment, the binding agent or antigen binding domain(s) thereof may comprise the amino acid sequence of the FR2 set forth in SEQ ID NO:121, or SEQ ID NO:180.

In another exemplary embodiment, the binding agent or antigen binding domain(s) thereof may comprise the amino acid sequence of the CDR3 set forth in SEQ ID NO:126 and the amino acid sequence of the FR2 set forth in SEQ ID NO:121.

In another exemplary embodiment, the binding agent or antigen binding domain(s) thereof may comprise the amino acid sequence of the CDR3 set forth in SEQ ID NO:185 and the amino acid sequence of the FR2 set forth in SEQ ID NO:180.

In another exemplary embodiment, the binding agent or antigen binding domain(s) thereof may comprise the amino acid sequence of the CDR3 set forth in SEQ ID NO:186 and the amino acid sequence of the FR2 set forth in SEQ ID NO:180.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR3 is as set forth in SEQ ID NO:126, SEQ ID NO:185 or SEQ ID NO:186 and the amino acid sequence of the FR2 is as set forth in SEQ ID NO:121 or SEQ ID NO:180. In accordance with the present disclosure, the amino acid sequence of the CDR3 and the amino acid sequence of the FR2 are on a same polypeptide chain. Alternatively, in accordance with the present disclosure, the amino acid sequence of the CDR3 and the amino acid sequence of the FR2 are on separate polypeptide chains.

In accordance with the present disclosure the amino acid sequence of the CDR3 is as set forth in SEQ ID NO:48, SEQ ID NO:51, SEQ ID NO:54, SEQ ID NO:58, SEQ ID NO:61, SEQ ID NO:220, SEQ ID NO:230, SEQ ID NO:238, SEQ ID NO:246, SEQ ID NO:303, SEQ ID NO:310, SEQ ID NO:317, SEQ ID NO:324, SEQ ID NO:331, SEQ ID NO:338, SEQ ID NO:345, SEQ ID NO:352, SEQ ID NO:396, SEQ ID NO:404, SEQ ID NO:411, SEQ ID NO:418, SEQ ID NO:425, SEQ ID NO:432, SEQ ID NO:439, SEQ ID NO:446, SEQ ID NO:453, SEQ ID NO:460, SEQ ID NO:467, SEQ ID NO:490, or SEQ ID NO:501.

In an exemplary embodiment, the single domain antibody or antigen binding fragment thereof may have the amino acid sequence as defined in any one of SEQ ID NOs: 143 to 146 or in SEQ ID NO:211 or 213, wherein the CDR3 amino acid sequence is as set forth in SEQ ID NO:48, SEQ ID NO:51, SEQ ID NO:54, SEQ ID NO:58, SEQ ID NO:61, SEQ ID NO:220, SEQ ID NO:230, SEQ ID NO:238, SEQ ID NO:246, SEQ ID NO:303, SEQ ID NO:310, SEQ ID NO:317, SEQ ID NO:324, SEQ ID NO:331, SEQ ID NO:338, SEQ ID NO:345, SEQ ID NO:352, SEQ ID NO:396, SEQ ID NO:404, SEQ ID NO:411, SEQ ID NO:418, SEQ ID NO:425, SEQ ID NO:432, SEQ ID NO:439, SEQ ID NO:446, SEQ ID NO:453, SEQ ID NO:460, SEQ ID NO:467, SEQ ID NO:490 or SEQ ID NO:501.

In accordance with the present disclosure the amino acid sequence of the FR2 is as set forth in SEQ ID NO:116, SEQ ID NO:117, SEQ ID NO:118, SEQ ID NO:119, SEQ ID NO:120, SEQ ID NO:354, SEQ ID NO:355, SEQ ID NO:356, SEQ ID NO:360, SEQ ID NO:361 or SEQ ID NO:505.

In an exemplary embodiment, the single domain antibody or antigen binding fragment thereof may have the amino acid sequence as defined in any one of SEQ ID NOs: 143 to 146 or in SEQ ID NO:211 or 213, wherein the FR2 amino acid sequence is as set forth in SEQ ID NO:116, SEQ ID NO:117, SEQ ID NO:118, SEQ ID NO:119, SEQ ID NO:120, SEQ ID NO:354, SEQ ID NO:355, SEQ ID NO:356, SEQ ID NO:360, SEQ ID NO:361 or SEQ ID NO:505.

The binding agent or antigen binding domain may comprise the amino acid sequence of the CDR1 set forth in SEQ ID NO:122, SEQ ID NO:181 or SEQ ID NO:182.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR1 is as set forth in SEQ ID NO:122, SEQ ID NO:181 or SEQ ID NO:182.

In accordance with the present disclosure, the amino acid sequence of the CDR1 is as set forth in SEQ ID NO:52, SEQ ID NO:55, SEQ ID NO:56, SEQ ID NO:59, SEQ ID NO:62, SEQ ID NO:218, SEQ ID NO: 228, SEQ ID NO: 236, SEQ ID NO:244, SEQ ID NO:301, SEQ ID NO:308, SEQ ID NO:315, SEQ ID NO:322, SEQ ID NO: 329, SEQ ID NO:336, SEQ ID NO:343, SEQ ID NO:350, SEQ ID NO:394, SEQ ID NO:402, SEQ ID NO:409, SEQ ID NO:416, SEQ ID NO:423, SEQ ID NO:430, SEQ ID NO:437, SEQ ID NO:444, SEQ ID NO:451, SEQ ID NO:458, SEQ ID NO:465, SEQ ID NO:488 or SEQ ID NO:500.

In an exemplary embodiment, the single domain antibody or antigen binding fragment thereof may have the amino acid sequence as defined in any one of SEQ ID NOs: 143 to 146 or in SEQ ID NO:211 or 213, wherein the CDR1 amino acid sequence is as set forth in SEQ ID NO:52, SEQ ID NO:55, SEQ ID NO:56, SEQ ID NO:59, SEQ ID NO:62, SEQ ID NO:218, SEQ ID NO: 228, SEQ ID NO: 236, SEQ ID NO:244, SEQ ID NO:301, SEQ ID NO:308, SEQ ID NO:315, SEQ ID NO:322, SEQ ID NO: 329, SEQ ID NO:336, SEQ ID NO:343, SEQ ID NO:350, SEQ ID NO:394, SEQ ID NO:402, SEQ ID NO:409, SEQ ID NO:416, SEQ ID NO:423, SEQ ID NO:430, SEQ ID NO:437, SEQ ID NO:444, SEQ ID NO:451, SEQ ID NO:458, SEQ ID NO:465, SEQ ID NO:488 or SEQ ID NO:500.

The binding agent or antigen binding domain may comprise the amino acid sequence of the CDR2 set forth in SEQ ID NO:125, SEQ ID NO:183 or SEQ ID NO:184.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR2 is as set forth in SEQ ID NO:125, SEQ ID NO:183 or SEQ ID NO:184.

In accordance with the present disclosure, the amino acid sequence of the CDR2 is as set forth in SEQ ID NO:47, SEQ ID NO:50, SEQ ID NO:53, SEQ ID NO:57, SEQ ID NO:60, SEQ ID NO:123, SEQ ID NO:124, SEQ ID NO:219, SEQ ID NO:229, SEQ ID NO:237, SEQ ID NO:245, SEQ ID NO:302, SEQ ID NO:309, SEQ ID NO:316, SEQ ID 323, SEQ ID NO:330, SEQ ID NO:337, SEQ ID NO:344, SEQ ID NO:351, SEQ ID NO:395, SEQ ID NO:403, SEQ ID NO:410, SEQ ID NO:417, SEQ ID NO:424, SEQ ID NO:431, SEQ ID NO:438, SEQ ID NO:445, SEQ ID NO:452, SEQ ID NO:459, SEQ ID NO:466, or SEQ ID NO:489.

In an exemplary embodiment, the single domain antibody or antigen binding fragment thereof may have the amino acid sequence as defined in any one of SEQ ID NOs: 143 to 146 or in SEQ ID NO:211 or 213, wherein the CDR2 amino acid sequence is as set forth in SEQ ID NO:47, SEQ ID NO:50, SEQ ID NO:53, SEQ ID NO:57, SEQ ID NO:60, SEQ ID NO:123, SEQ ID NO:124, SEQ ID NO:219, SEQ ID NO:229, SEQ ID NO:237, SEQ ID NO:245, SEQ ID NO:302, SEQ ID NO:309, SEQ ID NO:316, SEQ ID 323, SEQ ID NO:330, SEQ ID NO:337, SEQ ID NO:344, SEQ ID NO:351, SEQ ID NO:395, SEQ ID NO:403, SEQ ID NO:410, SEQ ID NO:417, SEQ ID NO:424, SEQ ID NO:431, SEQ ID NO:438, SEQ ID NO:445, SEQ ID NO:452, SEQ ID NO:459, SEQ ID NO:466 or SEQ ID NO:489.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR3 is as set forth in any one of SEQ ID NO:48, SEQ ID NO:51, SEQ ID NO:54, SEQ ID NO:58, SEQ ID NO:61, SEQ ID NO:220, SEQ ID NO:230, SEQ ID NO:238, SEQ ID NO:246, SEQ ID NO:303, SEQ ID NO:310, SEQ ID NO:317, SEQ ID NO:324, SEQ ID NO:331, SEQ ID NO:338, SEQ ID NO:345, SEQ ID NO:352, SEQ ID NO:396, SEQ ID NO:404, SEQ ID NO:411, SEQ ID NO:418, SEQ ID NO:425, SEQ ID NO:432, SEQ ID NO:439, SEQ ID NO:446, SEQ ID NO:453, SEQ ID NO:460, or SEQ ID NO:467 and the amino acid sequence of the FR2 is as set forth in any one of SEQ ID NO:116, SEQ ID NO:117, SEQ ID NO:118, SEQ ID NO:119, SEQ ID NO:120, SEQ ID NO:354, SEQ ID NO:355, SEQ ID NO:356 or SEQ ID NO:360 or SEQ ID NO:505.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR3 is as set forth in any one of SEQ ID NO:48, SEQ ID NO:51, SEQ ID NO:54, SEQ ID NO:58, SEQ ID NO:61, SEQ ID NO:220, SEQ ID NO:230, SEQ ID NO:238, SEQ ID NO:246, SEQ ID NO:303, SEQ ID NO:310, SEQ ID NO:317, SEQ ID NO:324, SEQ ID NO:331, SEQ ID NO:338, SEQ ID NO:345 or SEQ ID NO:352, SEQ ID NO:396, SEQ ID NO:404, SEQ ID NO:411, SEQ ID NO:418, SEQ ID NO:425, SEQ ID NO:432, SEQ ID NO:439, SEQ ID NO:446, SEQ ID NO:453, SEQ ID NO:460, or SEQ ID NO:467, the amino acid sequence of the FR2 is as set forth in any one of SEQ ID NO:116, SEQ ID NO:117, SEQ ID NO:118, SEQ ID NO:119, SEQ ID NO:120, SEQ ID NO:354, SEQ ID NO:355, SEQ ID NO:356, SEQ ID NO:360, SEQ ID NO:361, SEQ ID NO:505, the amino acid sequence of the CDR1 is as set forth in any one of SEQ ID NO: 52, SEQ ID NO: 55, SEQ ID NO: 56, SEQ ID NO:59, SEQ ID NO: 62, SEQ ID NO:218, SEQ ID NO: 228, SEQ ID NO: 236, SEQ ID NO:244, SEQ ID NO:301, SEQ ID NO:308, SEQ ID NO:315, SEQ ID NO:322, SEQ ID NO: 329, SEQ ID NO:336, SEQ ID NO:343, or SEQ ID NO:350, SEQ ID NO:394, SEQ ID NO:402, SEQ ID NO:409, SEQ ID NO:416, SEQ ID NO:423, SEQ ID NO:430, SEQ ID NO:437, SEQ ID NO:444, SEQ ID NO:451, SEQ ID NO:458, SEQ ID NO:465 or SEQ ID NO:488, and the amino acid sequence of the CDR2 is as set forth in any one of SEQ ID NO: 47, SEQ ID NO:50, SEQ ID NO:53, SEQ ID NO: 57, SEQ ID NO: 60, SEQ ID NO:123, SEQ ID NO:124, SEQ ID NO:219, SEQ ID NO:229, SEQ ID NO:237, SEQ ID NO:245, SEQ ID NO:302, SEQ ID NO:309, SEQ ID NO:316, SEQ ID 323, SEQ ID NO:330, SEQ ID NO:337, SEQ ID NO:344, SEQ ID NO:351, SEQ ID NO:395, SEQ ID NO:403, SEQ ID NO:410, SEQ ID NO:417, SEQ ID NO:424, SEQ ID NO:431, SEQ ID NO:438, SEQ ID NO:445, SEQ ID NO:452, SEQ ID NO:459 or SEQ ID NO:466.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR3 is as set forth in SEQ ID NO:142, SEQ ID NO:191 or SEQ ID NO:192.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the FR2 is as set forth in SEQ ID NO:175, SEQ ID NO:193 or SEQ ID NO:194.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR3 is as set forth in SEQ ID NO:142, SEQ ID NO:191 or SEQ ID NO:192 and the amino acid sequence of the FR2 is as set forth in SEQ ID NO:175, SEQ ID NO:193 or SEQ ID NO:194.

In an exemplary embodiment, the single domain antibody or antigen binding fragment thereof may have the amino acid sequence as defined in any one of SEQ ID NOs: 143 to 146 or in SEQ ID NO:211 or 213, wherein the CDR3 amino acid sequence is as set forth in SEQ ID NO:3, SEQ ID NO:6, SEQ ID NO:9, SEQ ID NO:12, SEQ ID NO:15, SEQ ID NO:141, SEQ ID NO: 217, SEQ ID NO: 227, SEQ ID NO:235, SEQ ID NO:243, SEQ ID NO:300, SEQ ID NO:307, SEQ ID NO:314, SEQ ID NO:321, SEQ ID NO:328, SEQ ID NO: 335, SEQ ID NO:342, SEQ ID NO:349, SEQ ID NO:393, SEQ ID NO:401, SEQ ID NO:408, SEQ ID NO:415, SEQ ID NO:422, SEQ ID NO:429, SEQ ID NO:436, SEQ ID NO:443, SEQ ID NO:450, SEQ ID NO:457, SEQ ID NO:464, SEQ ID NO:487 or SEQ ID NO:499.

In an exemplary embodiment, the single domain antibody or antigen binding fragment thereof may have the amino acid sequence as defined in any one of SEQ ID NOs: 143 to 146 or in SEQ ID NO:211 or 213, wherein the FR2 amino acid sequence is as set forth SEQ ID NO:170, SEQ ID NO:171, SEQ ID NO:172, SEQ ID NO:173, SEQ ID NO:174, SEQ ID NO:357, SEQ ID NO:358 or SEQ ID NO:359. In a specific embodiment, the amino acid sequence of the FR2 is as set forth in SEQ ID NO:170 or SEQ ID NO:171.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR1 is as set forth in SEQ ID NO:128, SEQ ID NO:187 or SEQ ID NO:188.

In an exemplary embodiment, the single domain antibody or antigen binding fragment thereof may have the amino acid sequence as defined in any one of SEQ ID NOs: 143 to 146 or in SEQ ID NO:211 or 213, wherein the CDR1 amino acid sequence is as set forth in SEQ ID NO:1, SEQ ID NO:4, SEQ ID NO:7, SEQ ID NO:10, SEQ ID NO:13, SEQ ID NO:127, SEQ ID NO:215, SEQ ID NO:225, SEQ ID NO: 233, SEQ ID NO:241, SEQ ID NO:298, SEQ ID NO:305, SEQ ID NO:312, SEQ ID NO:319, SEQ ID NO:326, SEQ ID NO:333, SEQ ID NO:340, SEQ ID NO:391, SEQ ID NO:399, SEQ ID NO:406, SEQ ID NO:413, SEQ ID NO:420, SEQ ID NO:427, SEQ ID NO:434, SEQ ID NO:441, SEQ ID NO:448, SEQ ID NO:455, SEQ ID NO:462, or SEQ ID NO:485.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR2 is as set forth in SEQ ID NO:140, SEQ ID NO:189 or SEQ ID NO:190.

In an exemplary embodiment, the single domain antibody or antigen binding fragment thereof may have the amino acid sequence as defined in any one of SEQ ID NOs: 143 to 146 or in SEQ ID NO:211 or 213, wherein the CDR2 amino acid sequence is as set forth in SEQ ID NO:2, SEQ ID NO:5, SEQ ID NO:8, SEQ ID NO:11, SEQ ID NO:14, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:216, SEQ ID NO:226, SEQ ID NO:234, SEQ ID NO:242, SEQ ID NO:299, SEQ ID NO:306, SEQ ID NO:313, SEQ ID NO: 320, SEQ ID NO:327, SEQ ID NO:334, SEQ ID NO:341, SEQ NO: 348, SEQ ID NO:392, SEQ ID NO:400, SEQ ID NO:407, SEQ ID NO:414, SEQ ID NO:421, SEQ ID NO:428, SEQ ID NO:435, SEQ ID NO:442, SEQ ID NO:449, SEQ ID NO:456 SEQ ID NO:463, SEQ ID NO:486, SEQ ID NO:492, SEQ ID NO:495 or SEQ ID NO:498.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR3 is as set forth in any one of SEQ ID NO:3, SEQ ID NO:6, SEQ ID NO:12, SEQ ID NO:15, SEQ ID NO:141, SEQ ID NO: 217, SEQ ID NO: 227, SEQ ID NO:235, SEQ ID NO:243, SEQ ID NO:300, SEQ ID NO:307, SEQ ID NO:314, SEQ ID NO:321, SEQ ID NO:328, SEQ ID NO: 335, SEQ ID NO: 342, SEQ ID NO: 349, SEQ ID NO:393, SEQ ID NO:401, SEQ ID NO:408, SEQ ID NO:415, SEQ ID NO:422, SEQ ID NO:429, SEQ ID NO:436, SEQ ID NO:443, SEQ ID No:450, SEQ ID NO:457, SEQ ID NO:464, SEQ ID NO:487 or SEQ ID NO:499 and the amino acid sequence of the FR2 is as set forth in any one of SEQ ID NO:170, SEQ ID NO:171, SEQ ID NO:172, SEQ ID NO:173, SEQ ID NO:174, SEQ ID NO:357, SEQ ID NO:358 or SEQ ID NO:359.

In an exemplary embodiment, the single domain antibody or antigen binding fragment thereof may have the amino acid sequence as defined in any one of SEQ ID NOs: 143 to 146 or in SEQ ID NO:211 or 213, wherein the amino acid sequence of the CDR3 is as set forth in any one of SEQ ID NO:3, SEQ ID NO:6, SEQ ID NO:9, SEQ ID NO:12, SEQ ID NO:15, SEQ ID NO:141, SEQ ID NO: 217, SEQ ID NO: 227, SEQ ID NO:235, SEQ ID NO:243, SEQ ID NO:300, SEQ ID NO:307, SEQ ID NO:314, SEQ ID NO:321, SEQ ID NO:328, SEQ ID NO: 335, SEQ ID NO: 342 or SEQ ID NO: 349, SEQ ID NO:393, SEQ ID NO:401, SEQ ID NO:408, SEQ ID NO:415, SEQ ID NO:422, SEQ ID NO:429, SEQ ID NO:436, SEQ ID NO:443, SEQ ID NO:450, SEQ ID NO:457, SEQ ID NO:464 SEQ ID NO:487 or SEQ ID NO:499, the amino acid sequence of the FR2 is as set forth in any one of SEQ ID NO:170, SEQ ID NO:171, SEQ ID NO:172, SEQ ID NO:173, SEQ ID NO:174, SEQ ID NO:357, SEQ ID NO:358 or SEQ ID NO:359, the amino acid sequence of the CDR1 is as set forth in any one of SEQ ID NO:1, SEQ ID NO:4, SEQ ID NO:7, SEQ ID NO:10, SEQ ID NO:13, SEQ ID NO:127, SEQ ID NO:215, SEQ ID NO:225, SEQ ID NO: 233, SEQ ID NO:241, SEQ ID NO:298, SEQ ID NO:305, SEQ ID NO:312, SEQ ID NO:319, SEQ ID NO:326, SEQ ID NO:333, SEQ ID NO:340, SEQ ID NO:391, SEQ ID NO:399, SEQ ID NO:406, SEQ ID NO:413, SEQ ID NO:420, SEQ ID NO:427, SEQ ID NO:434, SEQ ID NO:441, SEQ ID NO:448, SEQ ID NO:455, SEQ ID NO:462 or SEQ ID NO:485 and the amino acid sequence of the CDR2 is as set forth in any one of SEQ ID NO:2, SEQ ID NO:5, SEQ ID NO:8, SEQ ID NO:11, SEQ ID NO:14, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:216, SEQ ID NO:226, SEQ ID NO:234, SEQ ID NO:242, SEQ ID NO:299, SEQ ID NO:306, SEQ ID NO:313, SEQ ID NO: 320, SEQ ID NO:327, SEQ ID NO:334, SEQ ID NO:341, SEQ NO: 348, SEQ ID NO:392, SEQ ID NO:400, SEQ ID NO:407, SEQ ID NO:414, SEQ ID NO:421, SEQ ID NO:428, SEQ ID NO:435, SEQ ID NO:442, SEQ ID NO:449, SEQ ID NO:456, SEQ ID NO:463, SEQ ID NO:486, SEQ ID NO:492, SEQ ID NO:495 or SEQ ID NO:498.

In an aspect of the present disclosure, single domain antibody variants include those comprising an amino acid sequence at least 65%, 70%, 75%, 80% 85%, 90%, 95%, 99% identical to the amino acid sequence of the antigen binding domains, variable region or single domain antibodies exemplified herein.

Other exemplary embodiments of single domain antibody variants include those comprising an amino acid sequence at least 65% identical to the amino acid sequence set forth in any one of SEQ ID NOs:22-26, SEQ ID NOs: 73-114, SEQ ID NOs:221-224, SEQ ID NO:231, SEQ ID NO:232, SEQ ID NO:239, SEQ ID NO:240, SEQ ID NO:247, SEQ ID NO:304, SEQ ID NO:295, SEQ ID NO:296, SEQ ID NO:297, SEQ ID NO:311, SEQ ID NO:318, SEQ ID NO:325, SEQ ID NO:332, SEQ ID NO:339, SEQ ID NO:346, SEQ ID NO:353, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:22. More particularly, the single domain antibody or antigen binding fragment thereof comprises an amino acid sequence at least 95% identical, at least 99% identical or identical to SEQ ID NO:22.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:23. More particularly, the single domain antibody or antigen binding fragment thereof comprises an amino acid sequence at least 95% identical, at least 99% identical or identical to SEQ ID NO:23.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:24. More particularly, the single domain antibody or antigen binding fragment thereof comprises an amino acid sequence at least 95% identical, at least 99% identical or identical to SEQ ID NO:24.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:25. More particularly, the single domain antibody or antigen binding fragment thereof comprises an amino acid sequence at least 95% identical, at least 99% identical or identical to SEQ ID NO:25.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:26. More particularly, the single domain antibody or antigen binding fragment thereof comprises an amino acid sequence at least 95% identical, at least 99% identical or identical to SEQ ID NO:26.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:71. More particularly, the single domain antibody or antigen binding fragment thereof comprises an amino acid sequence at least 95% identical, at least 99% identical or identical to SEQ ID NO:71.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:72. More particularly, the single domain antibody or antigen binding fragment thereof comprises an amino acid sequence at least 95% identical, at least 99% identical or identical to SEQ ID NO:72.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:73. More particularly, the single domain antibody or antigen binding fragment thereof comprises an amino acid sequence at least 95% identical, at least 99% identical or identical to SEQ ID NO:73.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:74. More particularly, the single domain antibody or antigen binding fragment thereof comprises an amino acid sequence at least 95% identical, at least 99% identical or identical to SEQ ID NO:74.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:75. More particularly, the single domain antibody or antigen binding fragment thereof comprises an amino acid sequence at least 95% identical, at least 99% identical or identical to SEQ ID NO:75.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:76. More particularly, the single domain antibody or antigen binding fragment thereof comprises an amino acid sequence at least 95% identical, at least 99% identical or identical to SEQ ID NO:76.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:77. More particularly, the single domain antibody or antigen binding fragment thereof comprises an amino acid sequence at least 95% identical, at least 99% identical or identical to SEQ ID NO:77.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:78. More particularly, the single domain antibody or antigen binding fragment thereof comprises an amino acid sequence at least 95% identical, at least 99% identical or identical to SEQ ID NO:78.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:79. More particularly, the single domain antibody or antigen binding fragment thereof comprises an amino acid sequence at least 95% identical, at least 99% identical or identical to SEQ ID NO:79.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:80. More particularly, the single domain antibody or antigen binding fragment thereof comprises an amino acid sequence at least 95% identical, at least 99% identical or identical to SEQ ID NO:80.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:81. More particularly, the single domain antibody or antigen binding fragment thereof comprises an amino acid sequence at least 95% identical, at least 99% identical or identical to SEQ ID NO:81.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:82. More particularly, the single domain antibody or antigen binding fragment thereof comprises an amino acid sequence at least 95% identical, at least 99% identical or identical to SEQ ID NO:82.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:83. More particularly, the single domain antibody or antigen binding fragment thereof comprises an amino acid sequence at least 95% identical, at least 99% identical or identical to SEQ ID NO:83.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:84. More particularly, the single domain antibody or antigen binding fragment thereof comprises an amino acid sequence at least 95% identical, at least 99% identical or identical to SEQ ID NO:84.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:85. More particularly, the single domain antibody or antigen binding fragment thereof comprises an amino acid sequence at least 95% identical, at least 99% identical or identical to SEQ ID NO:85.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:86. More particularly, the single domain antibody or antigen binding fragment thereof comprises may comprise an amino acid sequence at least 95% identical, at least 99% identical or identical to SEQ ID NO:86.

In an exemplary embodiment, the single domain antibody or antigen binding fragment thereof comprises may comprise an amino acid sequence at least 70% identical, at least 75% identical, at least 80% identical, at least 85% identical, at least 90% identical, at least 95% identical, at least 99% identical to SEQ ID NO:87. More particularly, the single domain antibody or antigen binding fragment thereof comprises an amino acid sequence at least 95% identical, at least 99% identical or identical to SEQ ID NO:87.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:88. More particularly, the single domain antibody or antigen binding fragment thereof comprises an amino acid sequence at least 95% identical, at least 99% identical or identical to SEQ ID NO:88.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:89. More particularly, the single domain antibody or antigen binding fragment thereof comprises an amino acid sequence at least 95% identical, at least 99% identical or identical to SEQ ID NO:89.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:90. More particularly, the single domain antibody or antigen binding fragment thereof comprises an amino acid sequence at least 95% identical, at least 99% identical or identical to SEQ ID NO:90.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:91. More particularly, the single domain antibody or antigen binding fragment thereof comprises an amino acid sequence at least 95% identical, at least 99% identical or identical to SEQ ID NO:91.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:92. More particularly, the single domain antibody or antigen binding fragment thereof comprises an amino acid sequence at least 95% identical, at least 99% identical or identical to SEQ ID NO:92.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:93. More particularly, the single domain antibody or antigen binding fragment thereof comprises an amino acid sequence at least 95% identical, at least 99% identical or identical to SEQ ID NO:93.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:94. More particularly, the single domain antibody or antigen binding fragment thereof comprises an amino acid sequence at least 95% identical, at least 99% identical or identical to SEQ ID NO:94.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:95. More particularly, the single domain antibody or antigen binding fragment thereof comprises an amino acid sequence at least 95% identical, at least 99% identical or identical to SEQ ID NO:95.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:96. More particularly, the single domain antibody or antigen binding fragment thereof comprises an amino acid sequence at least 95% identical, at least 99% identical or identical to SEQ ID NO:96.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:97. More particularly, the single domain antibody or antigen binding fragment thereof comprises an amino acid sequence at least 95% identical, at least 99% identical or identical to SEQ ID NO:97.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:98. More particularly, the single domain antibody or antigen binding fragment thereof comprises an amino acid sequence at least 95% identical, at least 99% identical or identical to SEQ ID NO:98.

In an exemplary embodiment the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:99. More particularly, the single domain antibody or antigen binding fragment thereof comprises an amino acid sequence at least 95% identical, at least 99% identical or identical to SEQ ID NO:99.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:100. More particularly, the single domain antibody or antigen binding fragment thereof comprises an amino acid sequence at least 95% identical, at least 99% identical or identical to SEQ ID NO:100.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:101. More particularly, the single domain antibody or antigen binding fragment thereof comprises an amino acid sequence at least 95% identical, at least 99% identical or identical to SEQ ID NO:101.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:102. More particularly, the single domain antibody or antigen binding fragment thereof comprises an amino acid sequence at least 95% identical, at least 99% identical or identical to SEQ ID NO:102.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:103. More particularly, the single domain antibody or antigen binding fragment thereof comprises an amino acid sequence at least 95% identical, at least 99% identical or identical to SEQ ID NO:103.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:104. More particularly, the single domain antibody or antigen binding fragment thereof comprises an amino acid sequence at least 95% identical, at least 99% identical or identical to SEQ ID NO:104.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:105. More particularly, the single domain antibody or antigen binding fragment thereof comprises an amino acid sequence at least 95% identical, at least 99% identical or identical to SEQ ID NO:105.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:106. More particularly, the single domain antibody or antigen binding fragment thereof comprises an amino acid sequence at least 95% identical, at least 99% identical or identical to SEQ ID NO:106.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:107. More particularly, the single domain antibody or antigen binding fragment thereof comprises an amino acid sequence at least 95% identical, at least 99% identical or identical to SEQ ID NO:107.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:108. More particularly, the single domain antibody or antigen binding fragment thereof comprises an amino acid sequence at least 95% identical, at least 99% identical or identical to SEQ ID NO:108.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:109. More particularly, the single domain antibody or antigen binding fragment thereof comprises an amino acid sequence at least 95% identical, at least 99% identical or identical to SEQ ID NO:109.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:110. More particularly, the single domain antibody or antigen binding fragment thereof comprises an amino acid sequence at least 95% identical, at least 99% identical or identical to SEQ ID NO:110.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:111. More particularly, the single domain antibody or antigen binding fragment thereof comprises an amino acid sequence at least 95% identical, at least 99% identical or identical to SEQ ID NO:111.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:112. More particularly, the single domain antibody or antigen binding fragment thereof comprises an amino acid sequence at least 95% identical, at least 99% identical or identical to SEQ ID NO:112.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:113. More particularly, the single domain antibody or antigen binding fragment thereof comprises an amino acid sequence at least 95% identical, at least 99% identical or identical to SEQ ID NO:113.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:114. More particularly, the single domain antibody or antigen binding fragment thereof comprises an amino acid sequence at least 95% identical, at least 99% identical or identical to SEQ ID NO:114.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:115. More particularly, the single domain antibody or antigen binding fragment thereof comprises an amino acid sequence at least 95% identical, at least 99% identical or identical to SEQ ID NO:115.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:221. More particularly, the single domain antibody or antigen binding fragment thereof comprises an amino acid sequence at least 95% identical, at least 99% identical or identical to SEQ ID NO:221.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:222. More particularly, the single domain antibody or antigen binding fragment thereof comprises an amino acid sequence at least 95% identical, at least 99% identical or identical to SEQ ID NO:222.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:223. More particularly, the single domain antibody or antigen binding fragment thereof comprises an amino acid sequence at least 95% identical, at least 99% identical or identical to SEQ ID NO:223.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:224. More particularly, the single domain antibody or antigen binding fragment thereof comprises an amino acid sequence at least 95% identical, at least 99% identical or identical to SEQ ID NO:224.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:231. More particularly, the single domain antibody or antigen binding fragment thereof comprises an amino acid sequence at least 95% identical, at least 99% identical or identical to SEQ ID NO:231.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:232. More particularly, the single domain antibody or antigen binding fragment thereof comprises an amino acid sequence at least 95% identical, at least 99% identical or identical to SEQ ID NO:232.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:239. More particularly, the single domain antibody or antigen binding fragment thereof comprises an amino acid sequence at least 95% identical, at least 99% identical or identical to SEQ ID NO:239.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:240. More particularly, the single domain antibody or antigen binding fragment thereof comprises an amino acid sequence at least 95% identical, at least 99% identical or identical to SEQ ID NO:240.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:247. More particularly, the single domain antibody or antigen binding fragment thereof comprises an amino acid sequence at least 95% identical, at least 99% identical or identical to SEQ ID NO:247.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:295. More particularly, the single domain antibody or antigen binding fragment thereof comprises an amino acid sequence at least 95% identical, at least 99% identical or identical to SEQ ID NO:295.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:296. More particularly, the single domain antibody or antigen binding fragment thereof comprises an amino acid sequence at least 95% identical, at least 99% identical or identical to SEQ ID NO:296.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:297. More particularly, the single domain antibody or antigen binding fragment thereof comprises an amino acid sequence at least 95% identical, at least 99% identical or identical to SEQ ID NO:297.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:304. More particularly, the single domain antibody or antigen binding fragment thereof comprises an amino acid sequence at least 95% identical, at least 99% identical or identical to SEQ ID NO:304.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:311. More particularly, the single domain antibody or antigen binding fragment thereof comprises an amino acid sequence at least 95% identical, at least 99% identical or identical to SEQ ID NO:311.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:318. More particularly, the single domain antibody or antigen binding fragment thereof comprises an amino acid sequence at least 95% identical, at least 99% identical or identical to SEQ ID NO:318.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:325. More particularly, the single domain antibody or antigen binding fragment thereof comprises an amino acid sequence at least 95% identical, at least 99% identical or identical to SEQ ID NO:325.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:332. More particularly, the single domain antibody or antigen binding fragment thereof comprises an amino acid sequence at least 95% identical, at least 99% identical or identical to SEQ ID NO:332.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:339. More particularly, the single domain antibody or antigen binding fragment thereof comprises an amino acid sequence at least 95% identical, at least 99% identical or identical to SEQ ID NO:339.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:346. More particularly, the single domain antibody or antigen binding fragment thereof comprises an amino acid sequence at least 95% identical, at least 99% identical or identical to SEQ ID NO:346.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:353. More particularly, the single domain antibody or antigen binding fragment thereof comprises an amino acid sequence at least 95% identical, at least 99% identical or identical to SEQ ID NO:353. In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:362. More particularly, the single domain antibody or antigen binding fragment thereof comprises an amino acid sequence at least 95% identical, at least 99% identical or identical to SEQ ID NO:362.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:363. More particularly, the single domain antibody or antigen binding fragment thereof comprises an amino acid sequence at least 95% identical, at least 99% identical or identical to SEQ ID NO:363.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:364. More particularly, the single domain antibody or antigen binding fragment thereof comprises an amino acid sequence at least 95% identical, at least 99% identical or identical to SEQ ID NO:364.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:365. More particularly, the single domain antibody or antigen binding fragment thereof comprises an amino acid sequence at least 95% identical, at least 99% identical or identical to SEQ ID NO:365.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:366. More particularly, the single domain antibody or antigen binding fragment thereof comprises an amino acid sequence at least 95% identical, at least 99% identical or identical to SEQ ID NO:366.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:367. More particularly, the single domain antibody or antigen binding fragment thereof comprises an amino acid sequence at least 95% identical, at least 99% identical or identical to SEQ ID NO:367.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:368. More particularly, the single domain antibody or antigen binding fragment thereof comprises an amino acid sequence at least 95% identical, at least 99% identical or identical to SEQ ID NO:368.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:369. More particularly, the single domain antibody or antigen binding fragment thereof comprises an amino acid sequence at least 95% identical, at least 99% identical or identical to SEQ ID NO:369.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:370. More particularly, the single domain antibody or antigen binding fragment thereof comprises an amino acid sequence at least 95% identical, at least 99% identical or identical to SEQ ID NO:370.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:371. More particularly, the single domain antibody or antigen binding fragment thereof comprises an amino acid sequence at least 95% identical, at least 99% identical or identical to SEQ ID NO:371.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:372. More particularly, the single domain antibody or antigen binding fragment thereof comprises an amino acid sequence at least 95% identical, at least 99% identical or identical to SEQ ID NO:372.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:373. More particularly, the single domain antibody or antigen binding fragment thereof comprises an amino acid sequence at least 95% identical, at least 99% identical or identical to SEQ ID NO:373.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:374. More particularly, the single domain antibody or antigen binding fragment thereof comprises an amino acid sequence at least 95% identical, at least 99% identical or identical to SEQ ID NO:374.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:375. More particularly, the single domain antibody or antigen binding fragment thereof comprises an amino acid sequence at least 95% identical, at least 99% identical or identical to SEQ ID NO:375.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:376. More particularly, the single domain antibody or antigen binding fragment thereof comprises an amino acid sequence at least 95% identical, at least 99% identical or identical to SEQ ID NO:376.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:377. More particularly, the single domain antibody or antigen binding fragment thereof comprises an amino acid sequence at least 95% identical, at least 99% identical or identical to SEQ ID NO:377.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:378. More particularly, the single domain antibody or antigen binding fragment thereof comprises an amino acid sequence at least 95% identical, at least 99% identical or identical to SEQ ID NO:378.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:379. More particularly, the single domain antibody or antigen binding fragment thereof comprises an amino acid sequence at least 95% identical, at least 99% identical or identical to SEQ ID NO:379.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:380. More particularly, the single domain antibody or antigen binding fragment thereof comprises an amino acid sequence at least 95% identical, at least 99% identical or identical to SEQ ID NO:380.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:381. More particularly, the single domain antibody or antigen binding fragment thereof comprises an amino acid sequence at least 95% identical, at least 99% identical or identical to SEQ ID NO:381.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:382. More particularly, the single domain antibody or antigen binding fragment thereof comprises an amino acid sequence at least 95% identical, at least 99% identical or identical to SEQ ID NO:382.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:383. More particularly, the single domain antibody or antigen binding fragment thereof comprises an amino acid sequence at least 95% identical, at least 99% identical or identical to SEQ ID NO:383.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:384. More particularly, the single domain antibody or antigen binding fragment thereof comprises an amino acid sequence at least 95% identical, at least 99% identical or identical to SEQ ID NO:384.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:385. More particularly, the single domain antibody or antigen binding fragment thereof comprises an amino acid sequence at least 95% identical, at least 99% identical or identical to SEQ ID NO:385.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:386. More particularly, the single domain antibody or antigen binding fragment thereof comprises an amino acid sequence at least 95% identical, at least 99% identical or identical to SEQ ID NO:386.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:387. More particularly, the single domain antibody or antigen binding fragment thereof comprises an amino acid sequence at least 95% identical, at least 99% identical or identical to SEQ ID NO:387.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:388. More particularly, the single domain antibody or antigen binding fragment thereof comprises an amino acid sequence at least 95% identical, at least 99% identical or identical to SEQ ID NO:388.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:389. More particularly, the single domain antibody or antigen binding fragment thereof comprises an amino acid sequence at least 95% identical, at least 99% identical or identical to SEQ ID NO:389.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:390. More particularly, the single domain antibody or antigen binding fragment thereof comprises an amino acid sequence at least 95% identical, at least 99% identical or identical to SEQ ID NO:390.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:397. More particularly, the single domain antibody or antigen binding fragment thereof comprises an amino acid sequence at least 95% identical, at least 99% identical or identical to SEQ ID NO:397.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:398. More particularly, the single domain antibody or antigen binding fragment thereof comprises an amino acid sequence at least 95% identical, at least 99% identical or identical to SEQ ID NO:398.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:405. More particularly, the single domain antibody or antigen binding fragment thereof comprises an amino acid sequence at least 95% identical, at least 99% identical or identical to SEQ ID NO:405.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:412. More particularly, the single domain antibody or antigen binding fragment thereof comprises an amino acid sequence at least 95% identical, at least 99% identical or identical to SEQ ID NO:412.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:419. More particularly, the single domain antibody or antigen binding fragment thereof comprises an amino acid sequence at least 95% identical, at least 99% identical or identical to SEQ ID NO:419.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:426. More particularly, the single domain antibody or antigen binding fragment thereof comprises an amino acid sequence at least 95% identical, at least 99% identical or identical to SEQ ID NO:426.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:433. More particularly, the single domain antibody or antigen binding fragment thereof comprises an amino acid sequence at least 95% identical, at least 99% identical or identical to SEQ ID NO:433.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:440. More particularly, the single domain antibody or antigen binding fragment thereof comprises an amino acid sequence at least 95% identical, at least 99% identical or identical to SEQ ID NO:440.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:447. More particularly, the single domain antibody or antigen binding fragment thereof comprises an amino acid sequence at least 95% identical, at least 99% identical or identical to SEQ ID NO:447.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:454. More particularly, the single domain antibody or antigen binding fragment thereof comprises an amino acid sequence at least 95% identical, at least 99% identical or identical to SEQ ID NO:454.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:461. More particularly, the single domain antibody or antigen binding fragment thereof comprises an amino acid sequence at least 95% identical, at least 99% identical or identical to SEQ ID NO:461.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:468. More particularly, the single domain antibody or antigen binding fragment thereof comprises an amino acid sequence at least 95% identical, at least 99% identical or identical to SEQ ID NO:468.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:469. More particularly, the single domain antibody or antigen binding fragment thereof comprises an amino acid sequence at least 95% identical, at least 99% identical or identical to SEQ ID NO:469.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:470. More particularly, the single domain antibody or antigen binding fragment thereof comprises an amino acid sequence at least 95% identical, at least 99% identical or identical to SEQ ID NO:470.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:471. More particularly, the single domain antibody or antigen binding fragment thereof comprises an amino acid sequence at least 95% identical, at least 99% identical or identical to SEQ ID NO:471.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:472. More particularly, the single domain antibody or antigen binding fragment thereof comprises an amino acid sequence at least 95% identical, at least 99% identical or identical to SEQ ID NO:472.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:473. More particularly, the single domain antibody or antigen binding fragment thereof comprises an amino acid sequence at least 95% identical, at least 99% identical or identical to SEQ ID NO:473.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:491. More particularly, the single domain antibody or antigen binding fragment thereof comprises an amino acid sequence at least 95% identical, at least 99% identical or identical to SEQ ID NO:491.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:493. More particularly, the single domain antibody or antigen binding fragment thereof comprises an amino acid sequence at least 95% identical, at least 99% identical or identical to SEQ ID NO:493.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:496. More particularly, the single domain antibody or antigen binding fragment thereof comprises an amino acid sequence at least 95% identical, at least 99% identical or identical to SEQ ID NO:496.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:502. More particularly, the single domain antibody or antigen binding fragment thereof comprises an amino acid sequence at least 95% identical, at least 99% identical or identical to SEQ ID NO:502.

In an exemplary embodiment, the single domain antibody or antigen binding fragment thereof comprises an amino acid sequence at least 70% identical, at least 75% identical, at least 80% identical, at least 85% identical, at least 90% identical, at least 95% identical, at least 99% identical to SEQ ID NO:503. More particularly, the single domain antibody or antigen binding fragment thereof comprises an amino acid sequence at least 95% identical, at least 99% identical or identical to SEQ ID NO:503.

In an exemplary embodiment, the single domain antibody or antigen binding fragment thereof comprises an amino acid sequence at least 75%, 80% 85%, 90%, 95% identical to the amino acid sequence set forth in SEQ ID NO:29.

In an exemplary embodiment, the single domain antibody or antigen binding fragment thereof comprises an amino acid sequence at least 75%, 80% 85%, 90%, 95% identical to the amino acid sequence set forth in SEQ ID NO:30.

In an exemplary embodiment, the single domain antibody or antigen binding fragment thereof comprises an amino acid sequence at least 75%, 80% 85%, 90%, 95% identical to the amino acid sequence set forth in SEQ ID NO:31.

In an exemplary embodiment, the single domain antibody or antigen binding fragment thereof comprises an amino acid sequence at least 75%, 80% 85%, 90%, 95% identical to the amino acid sequence set forth in SEQ ID NO:32.

In an exemplary embodiment, the single domain antibody or antigen binding fragment thereof comprises an amino acid sequence at least 75%, 80% 85%, 90%, 95% identical to the amino acid sequence set forth in SEQ ID NO:33.

In an exemplary embodiment, the single domain antibody or antigen binding fragment thereof comprises an amino acid sequence at least 75%, 80% 85%, 90%, 95% identical to the amino acid sequence set forth in SEQ ID NO:34.

In an exemplary embodiment, the single domain antibody or antigen binding fragment thereof comprises an amino acid sequence at least 75%, 80% 85%, 90%, 95% identical to the amino acid sequence set forth in SEQ ID NO:35.

In an exemplary embodiment, the single domain antibody or antigen binding fragment thereof comprises an amino acid sequence at least 75%, 80% 85%, 90%, 95% identical to the amino acid sequence set forth in SEQ ID NO:44. In an exemplary embodiment, the single domain antibody or antigen binding fragment thereof comprises an amino acid sequence at least 75%, 80% 85%, 90%, 95% identical to the amino acid sequence set forth in SEQ ID NO:45.

In other exemplary embodiments, the single domain antibody comprises an amino acid sequence at least 75%, 80% 85%, 90%, 95% identical to the amino acid sequence set forth in SEQ ID NO:482.

In other exemplary embodiments, the single domain antibody comprises an amino acid sequence at least 75%, 80% 85%, 90%, 95% identical to the amino acid sequence set forth in SEQ ID NO:483.

In other exemplary embodiments, the single domain antibody comprises an amino acid sequence at least 75%, 80% 85%, 90%, 95% identical to the amino acid sequence set forth in SEQ ID NO:484.

In other exemplary embodiments, the single domain antibody comprises an amino acid sequence at least 75%, 80% 85%, 90%, 95% identical to the amino acid sequence set forth in SEQ ID NO:494.

In other exemplary embodiments, the single domain antibody comprises an amino acid sequence at least 75%, 80% 85%, 90%, 95% identical to the amino acid sequence set forth in SEQ ID NO:497.

In other exemplary embodiments, the single domain antibody comprises an amino acid sequence at least 75%, 80% 85%, 90%, 95% identical to the amino acid sequence set forth in SEQ ID NO:504.

In accordance with the present disclosure, the single domain antibody or antigen binding fragment thereof of the present disclosure may comprise a sequence made from the combination of the IMGT CDRs of a given single domain antibody disclosed herein and heavy chain variable region backbone having the amino acid sequence set forth in SEQ ID NO:211.

In accordance with the present disclosure, the single domain antibody or antigen binding fragment thereof of the present disclosure may comprise a sequence made from the combination of the IMGT CDRs of a given single domain antibody disclosed herein and heavy chain variable region backbone having the amino acid sequence set forth in SEQ ID NO:213.

In accordance with the present disclosure the single domain antibody may comprise an amino acid sequence at least 75%, 80% 85%, 90%, 95% identical to the amino acid sequence combination of Table 1A.

In accordance with the present disclosure the single domain antibody may comprise the amino acid sequence combination of Table 1A and a dimerization domain that comprises a) a sequence at least 80% identical to SEQ ID NO:38 and optionally to SEQ ID NO:37 and/or to SEQ ID NO:36 or to SEQ ID NO:481, b) a sequence at least 80% identical to SEQ ID NO:43 and optionally to SEQ ID NO:179 and/or to SEQ ID NO:42, c) a sequence at least 80% identical to SEQ ID NO: 212 or to SEQ ID NO:506, d) a sequence at least 80% identical to SEQ ID NO: 214, or e) swapping of elements of a) and b).

In accordance with the present disclosure, the single domain antibody or antigen binding fragment thereof comprises a sequence as set forth herein, wherein the amino acid residue at position 37 is V, the amino acid residue at position 39 is Q, the amino acid residue at position 45 is P, the amino acid residue at position 47 is W, the amino acid residue at position 50 is Y, the amino acid residue at position 58 is Y, the amino acid residue at position 99 is R, the amino acid residue at position 100 is L, the amino acid residue at position 101 is T, the amino acid residue at position 102 is D, the amino acid residue at position 103 is S, the amino acid residue at position 104 is H, the amino acid residue at position 105 is Y, the amino acid residue at position 106 is V, the amino acid residue at position 107 is E, the amino acid residue at position 108 is D, the amino acid residue at position 109 and/or the position is with reference to SEQ ID NO:145 (Table 1C).

In accordance with the present disclosure, the single domain antibody or antigen binding fragment thereof comprises a sequence as set forth herein, wherein the amino acid residue at position 1 is Q, the amino acid residue at position 2 is V, the amino acid residue at position 37 is V, the amino acid residue at position 39 is Q, the amino acid residue at position 45 is P, the amino acid residue at position 47 is W, the amino acid residue at position 50 is Y, the amino acid residue at position 58 is Y, the amino acid residue at position 99 is R, the amino acid residue at position 100 is L, the amino acid residue at position 101 is T, the amino acid residue at position 102 is D, the amino acid residue at position 103 is S, the amino acid residue at position 104 is H, the amino acid residue at position 105 is Y, the amino acid residue at position 106 is V, the amino acid residue at position 107 is E, the amino acid residue at position 108 is D, the amino acid residue at position 109 is A, the amino acid residue at position 110 is W and/or the amino acid residue at position 112 is Q and the position is with reference to SEQ ID NO:145 (Table 1C).

In accordance with the present disclosure, the single domain antibody or antigen binding fragment thereof comprises a sequence as set forth herein, wherein the amino acid residue at position 1 is Q, the amino acid residue at position 2 is V, the amino acid residue at position 3 is Q, the amino acid residue at position 4 is L, the amino acid residue at position 37 is V, the amino acid residue at position 39 is Q, the amino acid residue at position 45 is P, the amino acid residue at position 46 is E, the amino acid residue at position 47 is W, the amino acid residue at position 50 is Y, the amino acid residue at position 58 is Y, the amino acid residue at position 99 is R, the amino acid residue at position 100 is L, the amino acid residue at position 101 is T, the amino acid residue at position 102 is D, the amino acid residue at position 103 is S, the amino acid residue at position 104 is H, the amino acid residue at position 105 is Y, the amino acid residue at position 106 is V, the amino acid residue at position 107 is E, the amino acid residue at position 108 is D, the amino acid residue at position 109 is A, the amino acid residue at position 110 is W, the amino acid residue at position 111 is G, and/or the amino acid residue at position 112 is Q and the position is with reference to SEQ ID NO:145 (Table 1C).

In accordance with the present disclosure, the single domain antibody or antigen binding fragment thereof comprises a sequence as set forth herein with amino acid substitutions (conservative or non conservative).

In some embodiments, the single domain antibody or antigen binding fragment thereof comprises a sequence as set forth herein with amino acid substitutions selected from:
a) E at position 1, V at position 5, L at position 11, P at position 14, A at position 60, T at position 62, Q at position 81, S at position 84, R at position 86, A at position 87, V at position 92, Y at position 94,
b) V at position 5, L at position 11, P at position 14, T at position 62, Q at position 81, S at position 84, R at position 86, A at position 87, V at position 92, Y at position 94,
c) V at position 5, L at position 11, P at position 14, T at position 62, Q at position 81, S at position 84, R at position 86, A at position 87, V at position 92,
d) V at position 5, L at position 11, T at position 62, Q at position 81, S at position 84, V at position 92,
e) T at position 62, Q at position 81, S at position 84, V at position 92,
f) Q at position 81, S at position 84, V at position 92,
g) E at position 1, L at position 5, L at position 11, P at position 14, L at position 45, V at position 50, A at position 60, T at position 62, S at position 74, Q at position 81, S at position 84, R at position 86, A at position 87, V at position 92, Y at position 94, M at position 115,
h) L at position 11, P at position 14, T at position 62, S at position 74, Q at position 81, R at position 86, A at position 87, M at position 115,
i) L at position 11, P at position 14, L at position 45, A at position 60, T at position 62, S at position 74, Q at position 81, S at position 84, R at position 86, A at position 87, V at position 92, M at position 115,
j) E at position 1, L at position 11, P at position 14, L at position 45, A at position 60, T at position 62, S at position 74, Q at position 81, S at position 84, R at position 86, A at position 87, V at position 92, M at position 115,
k) L at position 5, L at position 11, P at position 14, L at position 45, A at position 60, T at position 62, S at position 74, Q at position 81, S at position 84, R at position 86, A at position 87, V at position 92, M at position 115,
l) L at position 11, P at position 14, L at position 45, V at position 50, A at position 60, T at position 62, S at position 74, Q at position 81, S at position 84, R at position 86, A at position 87, V at position 92, M at position 115,
m) L at position 11, P at position 14, L at position 45, A at position 60, T at position 62, S at position 74, Q at position 81, S at position 84, R at position 86, A at position 87, V at position 92, Y at position 94, M at position 115,
n) L at position 11, P at position 14, A at position 60, T at position 62, S at position 74, Q at position 81, S at position 84, R at position 86, A at position 87, V at position 92, M at position 115,
o) L at position 11, P at position 14, L at position 45, T at position 62, S at position 74, Q at position 81, S at position 84, R at position 86, A at position 87, V at position 92, M at position 115,
p) L at position 11, P at position 14, L at position 45, A at position 60, T at position 62, S at position 74, Q at position 81, R at position 86, A at position 87, V at position 92, M at position 115,
q) L at position 11, P at position 14, L at position 45, A at position 60, T at position 62, S at position 74, Q at position 81, S at position 84, R at position 86, A at position 87, M at position 115,
r) P at position 14, L at position 45, A at position 60, T at position 62, S at position 74, Q at position 81, S at position 84, R at position 86, A at position 87, V at position 92 or,
s) L at position 11, P at position 14, Q at position 44, R at position 45, A at position 60, T at position 62, S at position 74, Q at position 81, S at position 84, R at position 86, A at position 87, V at position 92, M at position 115.

In some embodiments, the single domain antibody or antigen binding fragment thereof comprises a sequence as set forth herein with amino acid substitutions selected from:
a) E at position 1, L at position 5, Q at position, 13 P at position 14, L at position 45, A at position 50, A at position 60, T at position 62, S at position 74, R at position 86, A at position 87, E at position 88, V at position 92, Y at position 94, L at position 115,
b) E at position 1, L at position 15, Q at position 13, P at position 14, L at position 45, A at position 60, T at position 62, S at position 74, R at position 86, A at position 87, E at position 88, V at position 92, Y at position 94, L at position 115,
c) E at position 1, L at position 5, Q at position 13, P at position 14, L at position 45, A at position 60, T at position 62, S at position 74, R at position 86, A at position 87, E at position 88, V at position 92, L at position 115,
d) L at position 5, Q at position 13, P at position 14, A at position 60, T at position 62, S at position 74, R at position 86, A at position 87, E at position 88, V at position 92, Y at position 94, L at position 115,
e) L at position 5, Q at position 13, P at position 14, A at position 60, T at position 62, S at position 74, R at position 86, A at position 87, E at position 88, V at position 92, L at position 115,
f) Q at position 13, P at position 14, T at position 62, S at position 74, R at position 86, A at position 87, E at position 88, V at position 92, Y at position 94,
g) Q at position 13, P at position 14, T at position 62, S at position 74, R at position 86, A at position 87, E at position 88, V at position 92,
h) Q at position 13, P at position 14, T at position 62, S at position 74, V at position 92,
i) Q at position 13, P at position 14, T at position 62,
j) E at position 1, V at position 5, I at position 12, Q at position 13, P at position 14, L at position 45, V at position 50, A at position 60, T at position 62, S at position 74, R at position 86, A at position 87, E at position 88, V at position 92, Y at position 94, L at position 115,
k) I at position 12, Q at position 13, P at position 14, T at position 62, S at position 74, R at position 86, A at position 87, L at position 115,
l) I at position 12, Q at position 13, P at position 14, L at position 45, A at position 60, T at position 62, S at position 74, R at position 86, A at position 87, E at position 88, V at position 92, L at position 115,
m) E at position 1, I at position 12, Q at position 13, P at position 14, L at position 45, A at position 60, T at position 62, S at position 74, R at position 86, A at position 87, E at position 88, V at position 92, L at position 115,
n) V at position 5, I at position 12, Q at position 13, P at position 14, L at position 45, A at position 60, T at position 62, S at position 74, R at position 86, A at position 87, E at position 88, V at position 92, L at position 115,
o) I at position 12, Q at position 13, P at position 14, L at position 45, V at position 50, A at position 60, T at position 62, S at position 74, R at position 86, A at position 87, E at position 88, V at position 92, L at position 115,
p) I at position 12, Q at position 13, P at position 14, L at position 45, A at position 60, T at position 62, S at position 74, R at position 86, A at position 87, E at position 88, V at position 92, Y at position 94, L at position 115,
q) I at position 12, Q at position 13, P at position 14, A at position 60, T at position 62, S at position 74, R at position 86, A at position 87, E at position 88, V at position 92, L at position 115,
r) I at position 12, Q at position 13, P at position 14, L at position 45, T at position 62, S at position 74, R at position 86, A at position 87, E at position 88, V at position 92, L at position 115,
s) I at position 12, Q at position 13, P at position 14, L at position 45, A at position 60, T at position 62, S at position 74, R at position 86, A at position 87, V at position 92, L at position 115,
t) I at position 12, Q at position 13, P at position 14, L at position 45, A at position 60, T at position 62, S at position 74, R at position 86, A at position 87, E at position 88, L at position 115 or,
u) I at position 12, P at position 14, L at position 45, A at position 60, T at position 62, S at position 74, R at position 86, A at position 87, E at position 88, V at position 92.

Yet other exemplary embodiments of single domain antibody variants include single domain antibody or antigen binding fragment thereof having an amino acid sequence as defined in any one of SEQ ID Nos:22-26, 73-114, 143 to 146, SEQ ID NOs:221-224, SEQ ID NO:231, SEQ ID NO:232, SEQ ID NO:239, SEQ ID NO:240, SEQ ID NO:247, SEQ ID NO:304, SEQ ID NO:295, SEQ ID NO:296, SEQ ID NO:297, SEQ ID NO:311, SEQ ID NO:318, SEQ ID NO:325, SEQ ID NO:332, SEQ ID NO:339, SEQ ID NO:346, SEQ ID NO:353, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503 and may comprise from one to five amino acid deletions or from one to five amino acid additions.

In an exemplary embodiment, the deletion or additions are consecutive. In another exemplary embodiment, the deletion or additions are non-consecutives. In yet another exemplary embodiment, the deletion or additions are at the N-terminus. In a further exemplary embodiment, the deletion or additions are at the C-terminus. In yet a further exemplary embodiment, the deletion or additions are outside of an IMGT CDR3 sequence and/or IMGT FR2 sequence. In yet a further exemplary embodiment, the deletion or additions are outside of a Kabat CDR3 sequence and/or Kabat FR2 sequence.

Exemplary embodiments of binding agent include for example and without limitation binding agent variants such as bispecific or trispecific binding agent variants.

For example, a bispecific binding agent variant may comprise one or more antigen binding domains of one or more single domain antibody variants.

In an exemplary embodiment, the binding agent is a bispecific or trispecific binding agent variant that comprises an antigen binding domain that specifically binds to CD47 and that comprises an amino acid sequence at least 80% identical, at least 85% identical, at least 90% identical, at least 95% identical, at least 99% identical to the amino acid sequence set forth in SEQ ID NO:155.

In an exemplary embodiment, the bispecific or trispecific binding agent variant is capable of binding to CD47 and TROP2 and comprises an amino acid sequence at least 80% identical, at least 85% identical, at least 90% identical, at least 95% identical, at least 99% to the amino acid sequence set forth in SEQ ID NO:155 and the amino acid sequence of an antigen binding domain of a single domain antibody or of a single domain antibody variant as disclosed herein.

The bispecific or trispecific binding agent variant may comprise:
a CDRH1 having the amino acid sequence set forth in SEQ ID NO156, a CDRH2 having the amino acid sequence set forth in SEQ ID NO:157 and a CDRH3 having the amino acid sequence set forth in SEQ ID NO:158, or
b. a CDRH1 having the amino acid sequence set forth in SEQ ID NO159, a CDRH2 having the amino acid sequence set forth in SEQ ID NO:160 and a CDRH3 having the amino acid sequence set forth in SEQ ID NO:161.

In another exemplary embodiment, the bispecific or trispecific binding agent variant comprises an antigen binding domain that specifically binds to PD-1 and that comprises an amino acid sequence at least 80% identical, at least 85% identical, at least 90% identical, at least 95% identical, at least 99% identical to the amino acid sequence set forth in SEQ ID NO:538.

In a further embodiment, the bispecific or trispecific binding agent variant may comprise
a CDRH1 having the amino acid sequence set forth in SEQ ID NO:532, a CDRH2 having the amino acid sequence set forth in SEQ ID NO:533 and a CDRH3 having the amino acid sequence set forth in SEQ ID NO:534, or
b. a CDRH1 having the amino acid sequence set forth in SEQ ID NO:535, a CDRH2 having the amino acid sequence set forth in SEQ ID NO:536 and a CDRH3 having the amino acid sequence set forth in SEQ ID NO:537.

### Competing Binding Agents

Binding agents of the present disclosure also encompass those that compete with a binding agent, or a single domain antibody disclosed herewith. Single domain antibodies that compete with one or more single domain antibody or antigen binding fragment thereof having an amino acid sequence as disclosed herein are provided.

In an exemplary embodiment the competing binding agent is capable of competing with a binding agent as disclosed herein.

In an exemplary embodiment the competing binding agent comprises one or more antigen binding domains that are capable of competing with the binding agent disclosed herein.

In an exemplary embodiment, a competing binding agent or competing single domain antibody may be selected based on any type of binding assays (FACS, ELISA, SPR etc.) using a TROP2 antigen as described herein.

In another exemplary embodiment, a competing binding agent or competing single domain antibody may be selected based on screening assays described herein.

In some exemplary embodiments, the competing binding agent or competing single domain antibody comprises an amino acid sequence at least 65% identical, at least 70% identical, at least 75% identical, at least 80% identical, at least 85% identical, at least 90% identical, at least 95% identical, at least 99% identical to the amino acid sequence set forth in any one of SEQ ID NOs:22-26, or SEQ ID NOs: 73-114, SEQ ID NOs:221-224, SEQ ID NO:231, SEQ ID NO:232, SEQ ID NO:239, SEQ ID NO:240, SEQ ID NO:247, SEQ ID NO:304, SEQ ID NO:295, SEQ ID NO:296, SEQ ID NO:297, SEQ ID NO:311, SEQ ID NO:318, SEQ ID NO:325, SEQ ID NO:332, SEQ ID NO:339, SEQ ID NO:346, SEQ ID NO:353, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503.

In other exemplary embodiments, the competing binding agent or competing single domain antibody may comprises amino acid residues of the CDR1, CDR2, CDR3 and/or FR2 of any one of SEQ ID NOs:22-26, or SEQ ID NOs: 73-114, SEQ ID NOs:221-224, SEQ ID NO:231, SEQ ID NO:232, SEQ ID NO:239, SEQ ID NO:240, SEQ ID NO:247, SEQ ID NO:304, SEQ ID NO:295, SEQ ID NO:296, SEQ ID NO:297, SEQ ID NO:311, SEQ ID NO:318, SEQ ID NO:325, SEQ ID NO:332, SEQ ID NO:339, SEQ ID NO:346, SEQ ID NO:353, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503.

### Nucleic Acids, Vectors, Kits, Cells and Method of Making Binding Agents

The present disclosure also relates to a nucleic acid molecule or vector encoding a binding agent or a binding agent variant as disclosed herein.

In some embodiments, the nucleic acid molecule or vector encodes an antibody or an antigen binding fragment thereof or a variant thereof.

In some embodiments, the nucleic acid molecule or vector encodes a single domain antibody or an antigen binding fragment thereof or a variant thereof.

In some embodiments, the nucleic acid molecule or vector encodes a competing binding agent.

In some embodiments, the nucleic acid molecule or vector encodes a competing antibody or antigen binding fragment thereof.

In some embodiments, the nucleic acid molecule or vector encodes a competing single domain antibody or antigen binding fragment thereof.

In some embodiments, the nucleic acid molecule or vector encodes an antibody-like molecule.

In some embodiments, the nucleic acid molecule or vector encodes a protein scaffold.

In some embodiments, the nucleic acid molecule or vector encodes an immune cell modulating agent.

Nucleic acid molecules of the present disclosure may be single-stranded or double-stranded. The nucleic acid molecules disclosed herein may comprises deoxyribonucleotides, ribonucleotides, modified deoxyribonucleotides or modified ribonucleotides. The nucleic acid molecules of the present disclosure may comprise for example DNA.

In some embodiments, the nucleic acid sequence is as set forth in Table 22.

Due to the inherent degeneracy of the genetic code, DNA sequences that encode the same, substantially the same or a functionally equivalent amino acid sequence may be produced and used. As such, in some embodiments, the nucleic acid sequence may be a variant of the nucleic acid sequence set forth in Table 22. In some embodiments, the nucleic acid variant may be at least 80%, at least 85%, at least 905, at least 95%, at least 99% identical to the nucleic acid sequence set forth in Table 22 and encodes an amino acid sequence of a binding agent, antigen binding domain or a portion disclosed herein.

The nucleotide sequences of the present disclosure may be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth. Codon-optimized nucleic acids encoding the polypeptide chains described herein are encompassed by the present disclosure.

The polypeptide chains and binding agents disclosed herein may be made by a variety of methods familiar to those skilled in the art, including by recombinant DNA methods or by in vitro transcription/translation.

Generally, the polypeptide chains described herein are expressed from nucleic acid sequences inserted into an expression vector, i.e., a vector that contains the elements for transcriptional and translational control of the inserted coding sequence in a particular host. These elements may include regulatory sequences, such as enhancers, constitutive and inducible promoters, and 5′ and 3′ un-translated regions.

A variety of expression vector/host cell systems known to those of skill in the art may be used to express the polypeptide chains or binding agent described herein. In the event, that the binding agent is composed of distinct polypeptide chains, each of such polypeptide chain may be provided by separate expression vectors or by a unique expression vector. In accordance with the present disclosure, the two chains of a binding agent may be encoded by a single vector or by separate vectors (vector set).

Polypeptides are often expressed in mammalian cells. For long-term production of recombinant proteins, a stable expression system may be used in which the DNA segment is incorporated into the host cell genome or maintained in an episomal form by the use of selectable markers. A host cell type may be chosen for its ability to modulate expression of the inserted sequences or to process the expressed polypeptide in the desired fashion. Different host cells that have specific cellular machinery and characteristic mechanisms for post-translational activities (e.g., CHO, HeLa, MDCK, HEK293, and W138) are available commercially and from the American Type Culture Collection (ATCC) and may be chosen to ensure the correct modification and processing of the expressed polypeptide.

Other types of expression system can be used. These include, for example, microorganisms such as bacteria transformed with recombinant bacteriophage, plasmid, or cosmid DNA expression vectors; yeast transformed with yeast expression vectors; insect cell systems infected with baculovirus vectors; plant cell systems transformed with viral or bacterial expression vectors; or animal cell systems.

The present disclosure therefore relates to isolated cells transformed or transfected with a vector, nucleic acid, sets of vectors or sets of nucleic acids encoding the binding agents (antibodies or antigen binding fragments thereof such as single domain antibodies) described herein. The present disclosure therefore also relates to isolated cells capable or expressing the binding agents (antibodies or antigen binding fragments such as single domain antibodies) thereof disclosed herein.

The present disclosure also relates to a method of making binding agents (e.g., antibodies or antigen binding fragments thereof such as single domain antibodies). The method may comprise providing a cell (e.g., a mammalian cell) with a vector or sets of vectors encoding the binding agent (e.g., antibodies or antigen binding fragments thereof such as single domain antibodies) disclosed herein and allowing expression.

The method may also comprises purifying the binding agents (e.g., antibodies or antigen binding fragments such as single domain antibodies) thereof from cells or cell debris.

In some embodiments, the method of manufacture allows to reach a purity level of at least 80%, of at least 85%, at least 90%, at least 99% (of dimers).

In some embodiments, the purity level is between 80.0% to 99.9%.

In some embodiments, the purity level is between 95.0% to 99.9%.

In some embodiments, the purity level is at least 90.0+/−5.0%.

In some embodiments, the purity level is at least 95.0+/−5.0%.

In some embodiments, the purity level is 99.0+/−1.0%.

In some embodiments, the titer of the antibodies or antigen binding fragments thereof (e.g., single domain antibody) produced by cells may be 0.1 g/L or more. In some instances, the titer of the antibodies or antigen binding fragments thereof produced by cells may be 0.5 g/L or more. In some instances, the titer of the antibodies or antigen binding fragments thereof produced by cells may be 1 g/L or more. In some instances, the titer of the antibodies or antigen binding fragments thereof produced by cells may be 2 g/L or more. In some instances, the titer of the antibodies or antigen binding fragments thereof produced by cells may be 3 g/L or more. In some instances, the titer of the antibodies or antigen binding fragments thereof produced by cells may be 4 g/L or more.

The method may also comprise a step of isolating and/or purifying the antibodies or antigen binding fragments thereof from impurities.

### Conjugates

In some embodiments, the binding agent of the present disclosure may be conjugated with a therapeutic moiety or with a detectable moiety.

Although, the binding agents of the present disclosure may have activity against tumor cells when unconjugated, the present disclosure also encompasses a conjugated form of such binding agents.

Binding agents described herein may, in some instances, have no or low activity against tumor cells when unconjugated. Binding agents may thus be conjugated with a therapeutic moiety so as to deliver the therapeutic moiety to TROP2-expressing cells.

The antibodies or antigen binding fragments thereof of the present disclosure (e.g., single domain antibodies or antigen binding fragments thereof) may be conjugated, for example, with a therapeutic moiety (for therapeutic purposes) or with a detectable moiety (i.e., for detection or diagnostic purposes) or to a protein allowing an extended half-life or is attached to nanoparticle.

In some instances, therapeutic or detectable moieties may be linked to at least one amino acid residues of the antibodies or antigen binding fragments thereof.

In an exemplary embodiment, the antibodies or antigen binding fragments thereof of the present disclosure is conjugated with a therapeutic moiety such as for example and without limitation, a chemotherapeutic, a cytokine, a cytotoxic agent, an anti-cancer drug (e.g., small molecule), and the like.

Therapeutic moiety may include, for example and without limitation, Yttrium-90, Scandium-47, Rhenium-186, Iodine-131, Iodine-125, and many others recognized by those skilled in the art (e.g., lutetium (e.g., Lu¹⁷⁷), bismuth (e.g., Bi²¹³), copper (e.g., Cu⁶⁷)), 5-fluorouracil, adriamycin, irinotecan, taxanes, pseudomonas endotoxin, ricin, auristatins (e.g., monomethyl auristatin E, monomethyl auristatin F), maytansinoids (e.g., mertansine, batansine), tubulysin B analogues (e.g., Tub196), camptothecin, exatecan derivatives (e.g., Dxd), belotecanderivatives, irinotecan or irinotecan metabolites (e.g., SN-38) and other toxins.

In another exemplary embodiment, the antibodies or antigen binding fragments thereof of the present disclosure is conjugated with a detectable moiety including for example and without limitation, a moiety detectable by spectroscopic, photochemical, biochemical, immunochemical, chemical and/or other physical means. A detectable moiety may be coupled either directly and/or indirectly (for example via a linkage, such as, without limitation, a DOTA or NHS linkage) to the polypeptide chain or binding agent using methods well known in the art. A wide variety of detectable moieties may be used, with the choice depending on the sensitivity required, ease of conjugation, stability requirements and available instrumentation. A suitable detectable moiety includes, but is not limited to, a fluorescent label, a radioactive label (for example, without limitation, ¹²⁵I, In¹¹¹, Tc⁹⁹, I¹³¹ and including positron emitting isotopes for PET scanner etc), a nuclear magnetic resonance active label, a luminescent label, a chemiluminescent label, a chromophore label, an enzyme label (for example and without limitation horseradish peroxidase, alkaline phosphatase, etc.), quantum dots and/or a nanoparticle. Detectable moiety may cause and/or produce a detectable signal thereby allowing for a signal from the detectable moiety to be detected.

### Pharmaceutical Compositions

Pharmaceutical compositions comprising binding agents of the present disclosure are also encompassed by the present disclosure.

In some embodiments, the pharmaceutical composition comprises a binding agent variant.

In some embodiments, the pharmaceutical composition comprises an antibody or an antigen binding fragment thereof or a variant thereof.

In some embodiments, the pharmaceutical composition comprises a single domain antibody or an antigen binding fragment thereof or a variant thereof.

In some embodiments, the pharmaceutical composition comprises a competing binding agent.

In some embodiments, the pharmaceutical composition comprises a competing antibody or antigen binding fragment thereof.

In some embodiments, the pharmaceutical composition comprises a competing single domain antibody or antigen binding fragment thereof.

In some embodiments, the pharmaceutical composition comprises an antibody-like molecule.

In some embodiments, the pharmaceutical composition comprises a protein scaffold.

In some embodiments, the pharmaceutical composition comprises an immune cell modulating agent or cells expressing the immune cell modulating agent.

Pharmaceutical compositions may therefore comprise the antibodies or antigen binding fragments thereof of the present disclosure (e.g., single domain antibodies or antigen. Binding fragments thereof).

The pharmaceutical composition of the present disclosure may also comprise a pharmaceutically acceptable carrier.

In some embodiments, the pharmaceutical composition comprises conjugated binding agents.

In some embodiments, the pharmaceutical composition comprises conjugated antibodies or antigen binding fragments thereof as disclosed herein (e.g., single domain antibodies or antigen binding fragment thereof). In some embodiments, the pharmaceutical composition comprises antibodies or antigen binding fragments thereof (e.g., single domain antibodies or antigen binding fragment thereof) conjugated with a therapeutic moiety. In some embodiments, the pharmaceutical composition comprises antibodies or antigen binding fragments thereof (e.g., single domain antibodies or antigen binding fragment thereof) conjugated with a detectable label.

In addition to the active ingredients, a pharmaceutical composition may contain pharmaceutically acceptable carriers comprising water, PBS, salt solutions, gelatins, oils, alcohols, and other excipients and auxiliaries that facilitate processing of the active compounds into preparations that may be used pharmaceutically. In other instances, such preparations may be sterilized.

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween™ 20, Tween™ 80, Pluronic F68, bile acid salts). Solubilizing agents (e.g., glycerol, polyethylene glycerol), antioxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also encompassed by the disclosure are particulate compositions coated with polymers (e.g., poloxamers or poloxamines). Other embodiments of the compositions of the disclosure incorporate particulate forms protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” or “pharmaceutical carrier” are known in the art and include, but are not limited to, 0.01-0.1 M or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the pharmaceutical compositions described above may be applied to any subject in need of therapy, including, but not limited to, mammals such as dogs, cats, cows, horses, rabbits, monkeys, and especially humans.

The pharmaceutical compositions described herein may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In an exemplary embodiment, the pharmaceutical composition comprises a single domain antibody or an antigen binding fragment thereof selected from KD001, KD002, KD003, KD004, KD005, KD006, KD007, KD008, KD009, KD010, KD011, KD012, KD013, KD014, KD015, KD016, KD017, KD018, KD019, KD020, KD021, KD022, KD023, KD024, KD025, KD026, KD027, KD028, KD029, KD030, KD031, KD032, KD033, KD034, KD035, KD036, KD037, KD038, KD039, KD040, KD041, KD042, KD043, KD044, KD045, KD046, KD047, KD048, KD049, KD050, KD051, KD052, KD053, KD054, KD055, KD056, KD057, KD058, KD059, KD060, KD061, KD062, KD063, KD064, KD067, KD068, KD069, KD071, KD072, KD073, KD074, KD075, KD076, KD077, KD078, KD079, KD080, KD081, KD082, KD083, KD084, KD085, KD086, KD087, KD088, KD089, KD090, KD091, KD092, KD093, KD094, KD095, KD096, KD097, KD098, KD099, KD100, KD101, KD102, KD103, KD104, KD105, KD106, KD107, KD108, KD109, KD110, KD111, KD112, KD113, KD114, KD115, KD116, KD117, KD118, KD119, DK120, KD121, KD122, KD123, KD124, KD125, KD126, KD127, KD128, KD129, KD130, KD131, KD132, KD133, KD134, KD135, KD136, KD137, KD138, KD139, KD140, or KD141 a variant (such as humanized variants) or their conjugated form thereof.

### Method of Use

The present disclosure also relates to the use of binding agents disclosed herein (or pharmaceutical composition comprising same) for treatment of disorders or diseases.

In some embodiments, the method comprises administering a binding agent or a variant thereof or a pharmaceutical composition comprising same to a subject in need.

In some embodiments, the binding agent is an antibody or an antigen binding fragment thereof or a variant thereof and the method comprises administering the antibody or an antigen binding fragment thereof or variant thereof or a pharmaceutical composition comprising same to a subject in need.

In some embodiments, the binding agent is a single domain antibody or an antigen binding fragment thereof or a variant thereof and the method comprises administering the single domain antibody or antigen binding fragment thereof or a variant thereof or a pharmaceutical composition comprising same to a subject in need.

In some embodiments, the binding agent is a competing binding agent and the method comprises administering the competing binding agent or a pharmaceutical composition comprising same to a subject in need.

In some embodiments, the binding agent is a competing antibody or antigen binding fragment thereof and the method comprises administering the competing antibody or antigen binding fragment thereof or a pharmaceutical composition comprising same to a subject in need.

In some embodiments, the binding agent is a competing single domain antibody or antigen binding fragment thereof and the method comprises administering the competing single domain antibody or antigen binding fragment thereof or a pharmaceutical composition comprising same to a subject in need.

In some embodiments, the binding agent is an antibody-like molecule and the method comprises administering the antibody-like molecule or a pharmaceutical composition comprising same to a subject in need.

In In some embodiments, the binding agent comprises a protein scaffold and the method comprises administering the protein scaffold or a pharmaceutical composition comprising same to a subject in need.

In some embodiments, the binding agent is an immune cell modulating agent and the method comprises administering an immune cell modulating agent or a pharmaceutical composition comprising same to a subject in need or cells modified to express the immune cell modulating agent thereof.

The binding agents of the present disclosure may be used for treatment of disorders or diseases. In some embodiments, the binding agent of the present disclosure is a single domain antibody be used for treating disorders or diseases.

In some embodiments, the binding agent may be used to target therapeutics and/or diagnostics to a target cell, circulating protein or tissue. Accordingly, in some embodiments, the binding agent of the present disclosure is a single domain antibody used to target therapeutics and/or diagnostics to a target cell, circulating protein or tissue.

In some embodiments, the binding agent may be conjugated with a therapeutic moiety and used for therapeutic methods. Accordingly, in some embodiments, the binding agent of the present disclosure is a single domain antibody used for therapeutic methods.

In some embodiments, the binding agent may be conjugated with a detectable moiety and used for detection or diagnostic methods. Accordingly, in some embodiments, the binding agent of the present disclosure is a single domain antibody used for detection or diagnostic methods.

In some embodiments, the binding agent (e.g., antibody or antigen binding fragment thereof such as, for example, single domain antibody or antigen binding fragment thereof) of the present disclosure may be used for targeting tumors in vivo.

In some embodiments, the binding agent (e.g., antibody or antigen binding fragment thereof such as, for example, single domain antibody or antigen binding fragment thereof) may be used for promoting tumor regression and/or reducing tumor volume in vivo.

The binding agent of the present disclosure (e.g., antibody or antigen binding fragment thereof such as, for example, single domain antibody or antigen binding fragment thereof) may thus be used for cancer treatment.

The method of the present disclosure may comprise a step of administering the binding agent (e.g., antibody or antigen binding fragment thereof such as, for example, single domain antibody or antigen binding fragment thereof) or a pharmaceutical composition comprising the binding agent to an individual in need.

In some embodiments, the binding agent (e.g., antibody or antigen binding fragment thereof such as, for example, single domain antibody or antigen binding fragment thereof) is administered in combination with a chemotherapeutic.

In accordance with the present disclosure, the individual in need may be a human. Further in accordance with the present disclosure, the individual in need may be an animal.

In some embodiment, treatment of disorders or diseases that are caused or associated with expression of TROP2 are particularly contemplated. In some embodiment, treatment of disorders or diseases that are caused or associated with overexpression of TROP2 are also particularly contemplated.

In some embodiments, the disorder or disease may be cancer.

In some embodiments, the binding agent of the present disclosure (including for example and without limitations, an antibody or antigen binding fragment thereof such as, for example, a single domain antibody or antigen binding fragment thereof) is administered intravenously, intra-muscularly, or by fusion.

In some embodiment, the antibodies or antigen binding fragments thereof (e.g., single domain antibody or antigen binding fragment thereof) is administered intravenously.

In some embodiment, the antibodies or antigen binding fragments thereof (e.g., single domain antibody or antigen binding fragment thereof) is administered intra-muscularly.

In some embodiment, the antibodies or antigen binding fragments thereof (e.g., single domain antibody or antigen binding fragment thereof) is administered by infusion.

In some embodiments, the binding agent (including for example and without limitations, an antibody or antigen binding fragment thereof such as, for example, a single domain antibody or antigen binding fragment thereof) is administered at a dose of approximately between 0.01 to 150 mg/kg, 0.01 to 100 mg/kg, 0.01 to 50 mg/kg, 0.05 to 150 mg/kg, 0.05 to 100 mg/kg, 0.05 to 50 mg/kg, 0.05 to 30 mg/kg, 0.05 to 10 mg/kg, 0.1 to 50 mg/kg, 0.1 to 30 mg/kg, 0.1 to 10m/kg, or 1.0 to 10 mg/kg.

In some embodiments, the binding agent (including for example and without limitations, an antibody or antigen binding fragment thereof such as, for example, a single domain antibody or antigen binding fragment thereof) is administered at a dose of approximately between 0.05 to 100 mg/kg.

In some embodiments, the binding agent (including for example and without limitations, an antibody or antigen binding fragment thereof such as, for example, a single domain antibody or antigen binding fragment thereof) is administered at a dose of approximately between 0.05 to 50 mg/kg.

In other embodiments, the binding agent (including for example and without limitations, an antibody or antigen binding fragment thereof such as, for example, a single domain antibody or antigen binding fragment thereof) is administered at a dose of approximately between 0.05 to 30 mg/kg.

In yet other embodiments, the binding agent (including for example and without limitations, an antibody or antigen binding fragment thereof such as, for example, a single domain antibody or antigen binding fragment thereof) is administered at a dose of approximately between 0.05 to 10 mg/kg.

In yet other embodiments, the binding agent (including for example and without limitations, an antibody or antigen binding fragment thereof such as, for example, a single domain antibody or antigen binding fragment thereof) is administered at a dose of approximately between 0.1 to 10 mg/kg.

Accordingly, in exemplary embodiments, the dose of the binding agent may be approximately 0.1 mg/kg. In other exemplary embodiments, the dose of the binding agent may be approximately 0.2 mg/kg. In yet other exemplary embodiments, the dose of the binding agent may be approximately 0.3 mg/kg. In other exemplary embodiments, the dose of the binding agent may be approximately 0.4 mg/kg. In other exemplary embodiments, the dose of the binding agent may be approximately 0.5 mg/kg. In yet other exemplary embodiments, the dose of the binding agent may be approximately 1 mg/kg. In further exemplary embodiments, the dose of the binding agent may be approximately 2 mg/kg. In yet further exemplary embodiments, the dose of the binding agent may be approximately 3 mg/kg. In additional exemplary embodiments, the dose of the binding agent may be approximately 4 mg/kg. In other exemplary embodiments, the dose of the binding agent may be approximately 5 mg/kg. In yet other exemplary embodiments, the dose of the binding agent may be approximately 6 mg/kg. In further exemplary embodiments, the dose of the binding agent may be approximately 7 mg/kg. In yet further exemplary embodiments, the dose of the binding agent may be approximately 8 mg/kg. In additional exemplary embodiments, the dose of the binding agent may be approximately 9 mg/kg. In other exemplary embodiments, the dose of the binding agent may be approximately 10 mg/kg. In yet other exemplary embodiments, the dose of the binding agent may be more than 10 mg/kg.

Accordingly, in exemplary embodiments, the dose of the single domain antibody may be approximately 0.1 mg/kg. In other exemplary embodiments, the dose of the single domain antibody may be approximately 0.2 mg/kg. In yet other exemplary embodiments, the dose of the single domain antibody may be approximately 0.3 mg/kg. In other exemplary embodiments, the dose of single domain antibody may be approximately 0.4 mg/kg. In other exemplary embodiments, the dose of the single domain antibody may be approximately 0.5 mg/kg. In yet other exemplary embodiments, the dose of the single domain antibody may be approximately 1 mg/kg. In further exemplary embodiments, the dose of the single domain antibody may be approximately 2 mg/kg. In yet further exemplary embodiments, the dose of the single domain antibody may be approximately 3 mg/kg. In additional exemplary embodiments, the dose of the single domain antibody may be approximately 4 mg/kg. In other exemplary embodiments, the dose of single domain antibody may be approximately 5 mg/kg. In yet other exemplary embodiments, the dose of the single domain antibody may be approximately 6 mg/kg. In further exemplary embodiments, the dose of the single domain antibody may be approximately 7 mg/kg. In yet further exemplary embodiments, the dose of the single domain antibody may be approximately 8 mg/kg. In additional exemplary embodiments, the dose of the single domain antibody may be approximately 9 mg/kg. In other exemplary embodiments, the dose of the single domain antibody may be approximately 10 mg/kg. In yet other exemplary embodiments, the dose of the single domain antibody may be more than 10 mg/kg.

In accordance with the present disclosure, the binding agent (including for example and without limitations, an antibody or antigen binding fragment thereof such as, for example, a single domain antibody or antigen binding fragment thereof) may be administered once weekly, more often than once a week (e.g., twice a week), once every two weeks, once every three weeks, once every month, or once every two months.

In accordance with the present disclosure, the binding agent (including for example and without limitations, an antibody or antigen binding fragment thereof such as, for example, a single domain antibody or antigen binding fragment thereof) may be administered once weekly.

In accordance with the present disclosure, the binding agent (including for example and without limitations, an antibody or antigen binding fragment thereof such as, for example, a single domain antibody or antigen binding fragment thereof) may be administered more often than once a week (e.g., twice a week).

Also in accordance with the present disclosure, the binding agent (including for example and without limitations, an antibody or antigen binding fragment thereof such as, for example, a single domain antibody or antigen binding fragment thereof) may be administered once every two weeks.

In accordance with the present disclosure, the binding agent (including for example and without limitations, an antibody or antigen binding fragment thereof such as, for example, a single domain antibody or antigen binding fragment thereof) may be administered once every three weeks.

In accordance with the present disclosure, the binding agent (including for example and without limitations, an antibody or antigen binding fragment thereof such as, for example, a single domain antibody or antigen binding fragment thereof) may be administered once every month.

The binding agent of the present disclosure particularly encompasses single domain antibodies which are administered to a subject having a disorder or disease associated with TROP2 expression or with TROP2 overexpression.

In some embodiments, the anti-TROP2 single domain antibodies disclosed herein is administered to a subject having cancer selected from epithetical cancer, lung cancer (such as small cell lung cancer, non small cell lung cancer), myeloma, prostate cancer, breast cancer (such as triple negative breast cancer), rectal cancer, pancreatic cancer, glioblastoma, cervical cancer, colorectal cancer, gastric cancer, ovarian cancer, thyroid cancer, stomach cancer, cancer of the urinary bladder, cancer of the uterus, esophageal cancer, head and neck cancer, blood cancer.

In other embodiments, the anti-TROP2 single domain antibodies disclosed herein is administered to a subject having a metastatic cancer.

In an exemplary embodiment, the method comprises administering a single domain antibody or an antigen binding fragment thereof or a pharmaceutical composition comprising same.

In some embodiments, the binding agent is a single domain antibody and is used at a dose of approximately between 0.1 to 10 mg/kg once a week. In other embodiments, the binding agent is a conjugated single domain antibody and is used at a dose of approximately between 0.1 to 10 mg/kg once a week.

The binding agent (e.g., antibodies or antigen binding fragments thereof) of the present disclosure may be used for detection purposes.

Detection of a particular target may be performed in vitro by contacting a sample, containing or suspected of containing the target with the binding agent (e.g., antibodies or antigen binding fragments thereof) described herein and quantifying a signal associated with positive or negative binding using a detection apparatus.

The sample may originate from a mammal (e.g., a human). The sample may be a tissue sample obtained from the mammal or a cell culture supernatant.

In some embodiments, the sample may be a serum sample, a plasma sample, a blood sample, semen or ascitic fluid obtained from the mammal.

Detection of a particular target may be performed in vivo by administering the antibodies or antigen binding fragments thereof to an individual and quantifying a signal associated with positive or negative binding using a detection apparatus.

Upon detecting the presence of the target in the sample or in the individual, a drug (e.g., antibody, small molecule, antibodies or antigen binding fragments thereof disclosed herein) may be administered to the individual.

### Companion Diagnostic

The present application also provides a test and medical device that may be used as a companion diagnostic.

The test may be used to evaluate the expression of TROP2 in tumor or tumor cells and/or the presence of macrophages in a tumor.

In some embodiments, the test may be carried out prior to treating a subject with a drug targeting TROP2. Accordingly, the test may be carried out to determine whether a subject would be eligible for treatment with the drug.

In other embodiments, the test may be carried out during or subsequent to treatment with a drug targeting TROP2. Accordingly, the test may be carried out to determine whether the subject respond to treatment or not and/or to monitor ongoing treatment.

In some embodiments, the test may be used as a companion diagnostic to treatment with any type of drug targeting TROP2 such as for example and without limitation, small molecules and biologics including the binding agents disclosed herein or any therapeutic antibodies against TROP2.

In some embodiment, the test comprises a binding agent for detecting TROP2 in tumors or blood (e.g., in vivo or ex vivo). The test may also comprise a binding agent for detecting tumor macrophages.

The test may be carried out, for example, on a tumor biopsy obtained from a patient and the level of selected biomarker expression may assessed. In an embodiment, expression of TROP2 is determined and may be scored as low, moderate or high by IHC staining with the TROP2 binding agent. In another embodiment, the human macrophage infiltration, is determined and may be scored as low, moderate or high by IHC staining. The characterization of TROP2 expression or macrophage infiltration may be determined, for example, by a trained pathologist.

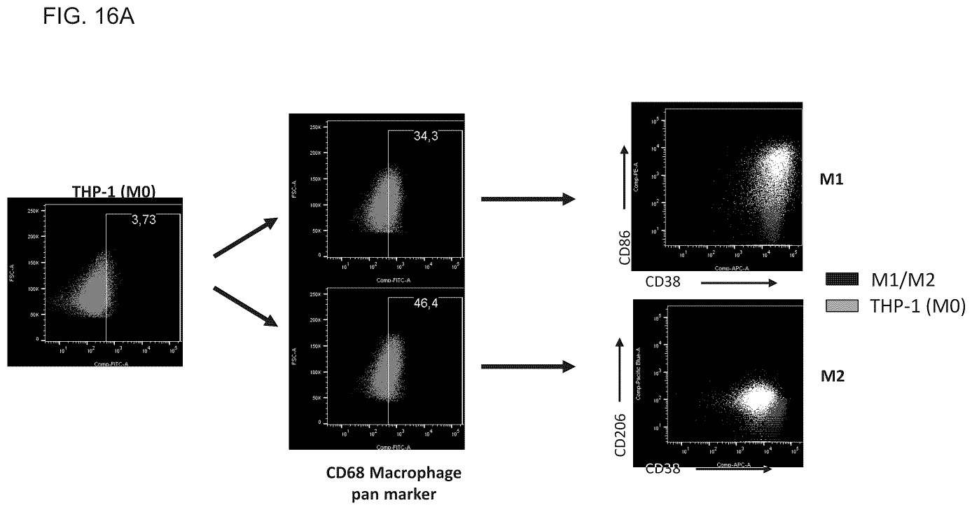

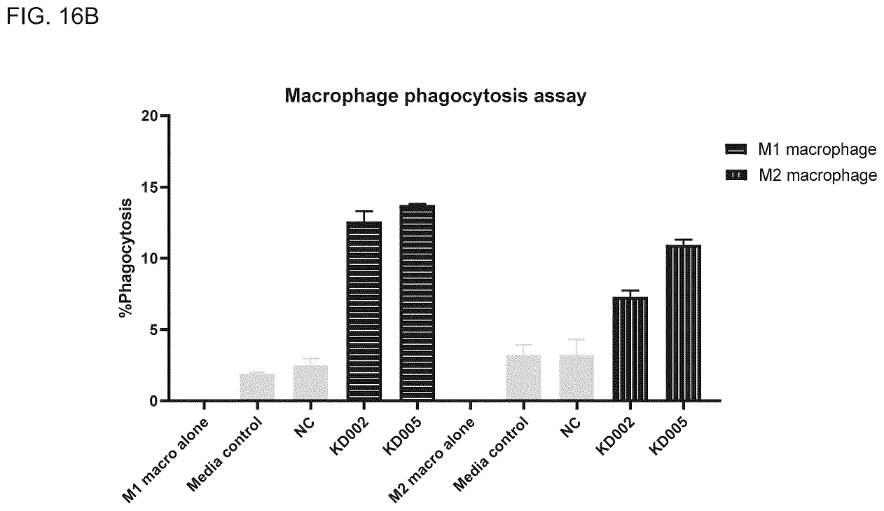

Based on the FIG. 16B indicating that both M1 and M2 macrophages lead to phagocytosis, a pan macrophage marker may be used to evaluate the macrophage infiltration level and the potential efficacy prediction. In an exemplary embodiment, the presence of macrophages in tumors may be assessed with the pan marker CD68.

The binding agent or pharmaceutical composition of the present disclosure may be administered to patients carrying tumors with high TROP2/high CD68 expression, low CD68/high TROP2 expression, low CD68/low TROP2 expression.

The present disclosure therefore relates to a kit that comprise a binding agent that specifically binds to TROP2 and a binding agent that binds to a macrophage marker.

The kit may more particularly comprise a binding agent that specifically binds to TROP2 and a binding agent that binds to a pan macrophage marker. Exemplary embodiments of pan macrophages markers include for example and without limitations, CD68, CD80, and CD11c.

### Screening Assay

Binding agents having same or similar property(ies) as any of the single domain antibodies can be identify by performing one or more of the assays described herein.

For example, candidate binding agents may be submitted to binding assays (e.g., ELISA, FACS etc.) for identifying those that bind to the same or overlapping region of TROP2 as any one of the single domain antibodies of the present disclosure.

The binding assay may measure positive binding to a specific region of TROP2 or to specific amino acid residues of TROP2 or loss of binding when a specific region of TROP2 is deleted or when specific amino acid residues of TROP2 are mutated or deleted.

The binding assays may be carried out on recombinant polypeptides or on cells expressing the polypeptides.

In some embodiments, a binding agent may be identified by contacting a candidate binding agent with a polypeptide comprising a sequence at least 80% identical, at least 85% identical, at least 90% identical, at least 95% identical, at least 99% identical or identical to the human TROP2 amino acid sequence, or a fragment thereof or with a cell expressing such polypeptide or fragment thereof.

Due to their high level of identity, a binding agent that binds to human TROP2 may be identified by contacting a candidate binding agent with a polypeptide comprising a cynomolgus TROP2 amino acid sequence or a fragment thereof or with a cell expressing such polypeptide or fragment thereof.

In an exemplary embodiment, the candidate binding agent is contacted with a polypeptide comprising a sequence consisting of amino acid residues 27-73 of human TROP2 or of cynomolgus TROP2 or a fragment thereof or with a cell expressing such polypeptide or fragment thereof.

In an exemplary embodiment, the candidate binding agent is contacted with a polypeptide comprising a sequence consisting of amino acid residues 27-56 of human TROP2 or of cynomolgus TROP2 or a fragment thereof or with a cell expressing such polypeptide or fragment thereof.

In an exemplary embodiment, the candidate binding agent is contacted with a polypeptide comprising a sequence consisting of amino acid residues 27-66 of human TROP2 or of cynomolgus TROP2 or a fragment thereof or with a cell expressing such polypeptide or fragment thereof.

The present disclosure therefore relates to a method of identifying a binding agent that comprises a) contacting a candidate binding agent with a polypeptide comprising i) a TROP2 amino acid sequence consisting of amino acid residues 27-146 of human TROP2 or of cynomolgus TROP2, ii) a polypeptide comprising a sequence at least 80% identical to the TROP2 amino acid sequence of i) or iii) a fragment of i) or ii) thereof and b) selecting a binding agent that binds to the polypeptide. The method of the present disclosure may also be carried out by contacting a candidate binding agent with cell expressing such polypeptide or fragment thereof.

The binding agent may also be selected for not being capable of binding to a polypeptide that comprises a human TROP2 amino acid sequence consisting of amino acid residues 147-274 or a fragment thereof or for having a reduced binding to said polypeptide compared to the full human TROP2 polypeptide.

For example, the binding agent may be selected for not being capable of binding to one or more polypeptides comprising a human TROP2 amino acid sequence carrying from 5 to 70 amino acid deletions in the amino acid sequence defined by residues 1-73 of human TROP2 or for having a reduced binding to said polypeptide compared to a human TROP2 polypeptide having no such deletion.

For example, the binding agent may be selected for not being capable of binding to one or more polypeptides selected from polypeptides comprising a human TROP2 amino acid sequence carrying deletion of amino acid residues 27-36, 27-44, 27-56, 27-66 or 27-73 or for having a reduced binding to said polypeptide compared to a human TROP2 polypeptide having no such deletion.

More particularly, the binding agent may be selected for not being capable of binding to a polypeptide comprising the amino acid sequence set forth in SEQ ID NO: 149.

Binding agents may also be selected based on their ability to compete with one or more binding agents or antigen binding domains disclosed herein. More particularly, binding agents may also be identified based on their ability to compete with any one of the single antibodies of the present disclosure.

In some embodiments, the binding agent or antigen binding domain does not compete with Sacituzumab, hTINA-1, h7E6, and/or AR47A6.4.2.

For example, the method of the present disclosure may comprise a) performing a competition assay between a candidate binding agent and the binding agent of any one of SEQ ID NO:22-26 or SEQ ID NO:94-114 for binding to i) human TROP2, ii) cynomolgus TROP2, iii) a polypeptide comprising a sequence at least 80% identical to human TROP2, iv) a polypeptide comprising a sequence at least 80% identical to cynomolgus TROP2 or v) a fragment of any one of i) to iv) thereof and b) selecting a competing binding agent.

In an exemplary embodiment, the competition assay is performed with a polypeptide comprising a sequence consisting of amino acid residues 27-146 of human TROP2 or of cynomolgus TROP2 or a fragment thereof.

In an exemplary embodiment, the competition assay is performed with a polypeptide comprising a sequence consisting of amino acid residues 27-73 of human TROP2 or of cynomolgus TROP2 or a fragment thereof.

In an exemplary embodiment, the competition assay is performed with a polypeptide comprising a sequence consisting of amino acid residues 27-56 of human TROP2 or of cynomolgus TROP2 or a fragment thereof.

In an exemplary embodiment, the competition assay is performed with a polypeptide comprising a sequence consisting of amino acid residues 27-66 of human TROP2 or of cynomolgus TROP2 or a fragment thereof.

In an exemplary embodiment, the TROP2 amino acid sequence used in the binding experiments and/or in the competition assay is a human TROP2 amino acid sequence.

In an exemplary embodiment, the TROP2 amino acid sequence used in the binding experiments and/or in the competition assay is a cynomolgus TROP2 amino acid sequence.

In an exemplary embodiment, the TROP2 fragment used in the binding experiments and/or in the competition assay is a fragment of at least 10 amino acid residues.

In an exemplary embodiment, the TROP2 fragment used in the binding experiments and/or in the competition assay is a fragment of between 10 to 30 amino acid residues, including for example a fragment of between 10 to 25 amino acid residues, a fragment of between 10 to 20 amino acid residues, a fragment of between 10 to 15 amino acid residues etc.

In an embodiment, the human TROP2 amino acid sequence used in the binding experiments and/or in the competition assay may include for example, amino acid residues 47-56 of human TROP2.

In another embodiment, the human TROP2 amino acid sequence used in the binding experiments and/or in the competition assay may include for example, amino acid residues 60-66 of human TROP2.

In yet another embodiment, the human TROP2 amino acid sequence used in the binding experiments and/or in the competition assay may include for example, amino acid residues 47-66 of human TROP2.

The candidate binding agent may comprise the antigen binding domain of an antibody or antigen binding fragment thereof, of an antibody-like molecule, of a fusion with protein scaffolds or of an immune cell modulating agent or the like.

Alternatively, the candidate binding agent may be selected from an antibody or antigen binding fragment thereof, an antibody-like molecule, a fusion with protein scaffolds or an immune cell modulating agent.

In an exemplary embodiment, the candidate binding agent may comprise an antibody having a light chain variable region and a heavy chain variable region or an antigen binding domain of said antibody.

In another exemplary embodiment, the candidate binding agent may comprise a heavy chain only antibody or an antigen binding domain of said heavy chain only antibody.

In some embodiments, the binding agent is selected for its affinity for human TROP2.

In some embodiments, the binding agent is selected for its affinity for cynomolgus TROP2.

In some embodiments, the binding agent is selected for having an affinity for human TROP2 that is similar (with +/−25% variation) to that of the single domain antibodies of the present disclosure.

In other embodiments, the binding agent is selected for having an affinity for human TROP2 that is identical or superior to that of single domain antibodies of the present disclosure.

In some embodiments, the binding agent is selected for having a dissociation rate that is similar (with +/−25% variation) to that of single domain antibodies of the present disclosure for human TROP2.

In some embodiments, the binding agent is selected for having a dissociation rate that is identical or lower to that of single domain antibodies of the present disclosure for human TROP2.

In accordance with the present disclosure, the affinity may be measured on recombinant human TROP2 protein or on cells expressing human TROP2 protein. The affinity may be measured on full length human TROP2 protein.

The binding agents can also be submitted to in vitro or in vivo assays to assess function. Exemplary assays include internalization, ADCC, ADCP, cell viability, in vitro tumor growth inhibition experiments and/or in vivo tumor growth inhibition experiments as described herein.

In some embodiments, the binding agent is selected for in vitro potency against tumor cells.

In some embodiments, the binding agent is selected for in vivo potency against tumor cells.

### EXAMPLES

### Example 1—Animal Immunization and Immune Library Construction

Single domain antibodies were generated by immunizing animals such as alpacas (Vicugna pacos) or llamas (llama glama) or transgenic mouse with human antigens, cDNA plasmid encoding the human antigens and/or cells overexpressing the human antigens. In some instances the extracellular domain of human TROP2 protein (aa 1-274) comprising His tag (e.g., SEQ ID NO:164) (Sino Biological, Cat. No. 10428-H08H: GenBank Acc. No. NP_002344.2)) cDNA or cells overexpressing same was used as antigen.

In some instances, Alpacas (Vicugna pacos) received cDNA encoding the whole human TROP2 protein (including ECD+TM+C) (~1 mg) and TROP2 Protein (ECD portion) (10 ug). For immunization with DNA samples, DNA solutions were injected intradermally at multiple sites at front and back limbs near the draining lymph nodes followed by electroporation. For immunization with proteins and/or cells, a mix of the protein and Gerbu adjuvant P or cells & Gerbu adjuvant P was injected subcutaneously at the end of neck (cranially or caudally to the scapula bone) and back limbs. Prior to injection, the cells were washed in PBS to remove the medium and DMSO. Before the start of the immunization, 10 ml of pre-immune blood was taken, which was processed to serum sample and stored at −80° C. Each animal received 6 bi-weekly injections. Five days after the last immunization, about 100 ml of blood was taken per animal for the preparation of peripheral lymphocytes (PBLs). The animals were also operated to remove draining lymph nodes downstream of the injection sites (for an overview of immunization schemes is illustrated below). The general health status (eating habit, body temperature, etc.) was monitored daily throughout the immunization period. The animals remained healthy during the whole procedure.

| Day | Immunization | Tissue collection |
|---|---|---|
| 0 | DNA immunization | 10 ml pre-immune blood |
| 14 | DNA immunization + | — |
| | Protein injection | |
| 28 | DNA immunization + | — |
| | Protein & cell injection | |
| 42 | DNA immunization + | — |
| | Protein & cell injection | |
| 56 | DNA immunization + | — |
| | Protein & cell injection | |
| 70 | DNA immunization + | — |
| | Protein & cell injection | |
| 75 | | 100 ml blood (PBL) |
| | | Lymph nodes |

Sequences of single-domain antibodies were uncovered from clone picking after phage display panning of reconstituted immune libraries against the antigens.

Briefly, single-domain antibody phage display libraries generated from immunized animals were panned against the antigen of interest for a total of four rounds: antigen-binding phages were first selected with three rounds of panning on recombinant human protein, followed by one round with a cell line expressing the antigen of interest (e.g., NCI-H292 or others). Individual clones were randomly isolated from the phages recovered from the final round of panning, and sequence of the antibody variable domains obtained by Sanger sequencing.

DNA fragments encoding VHHs or VHs or humanized versions were generally subcloned into constructs comprising a constant region of a human antibody (e.g., human IgG1 Fc) for expression and in vitro and in vivo testing.

Single domain antibodies comprising variable regions as set forth in Table 15A to Table 15K, Table 16, Table 18 or Table 19 are obtained and tested as described herein.

### Example 2—Production and Purification of sdAbs

sdAbs were expressed in cells and isolated as protein dimers. The sdAbs identified in the experimental section and/or figures as KD001 to KD141 are composed of two heavy chains. Unless, specifically mentioned otherwise each of KD001 to KD141 have the corresponding heavy chain (or variable region) amino acid sequence set forth in in Table 15A to Table 15K, Table 16, Table 18 or Table 19.

Protein dimers (e.g., homodimers) were expressed in 2.5 mL or 400 mL culture volume from the DNA construct using the ExpiCHO™ Expression System (Thermo Fisher, Cat. no. A29133) or the Expi293™ Expression System (Thermo Fisher, Cat. no. A14635).

Freshly thawed ExpiCHO-s cells were allowed to recover in culture for two or more passages before transfection. Cells were then passaged every 3-4 days until they reach 4×10⁶-6×10⁶ cells/mL at which time they were diluted to 2×10⁵-3×10⁵ cells/mL in ExpiCHO™ Expression Medium pre-warmed to 37° C. The day prior to transfection, cells were diluted to 3×10⁶-4×10⁶ cells/mL and on the day of transfection, cells were further diluted to 6×10⁶ cells/mL. 1 μg of DNA/mL of culture volume was diluted with cold OptiPRO™ medium (100 μL for 2.5 mL of culture volume; 16 mL for 400 mL of culture volume). ExpiFectamine™ CHO Reagent (8 μL for 2.5 mL of culture volume; 1280 μL for 400 mL of culture volume) was added to medium containing DNA and incubated with ExpiFectamine™/DNA complexes at room temperature for 1-5 min. Then the DNA complex was transferred to culture (at 6×10⁶ cells/mL) while swirling. The cells were incubated at 37° C. under 8% CO₂ and 80% humidity with shaking (INFORS HT shaker, 125 rpm). 18-22 h after onset of transfection, ExpiCHO™ feed (0.6 mL for 2.5 mL of culture volume; 96 mL for 400 mL of culture volume) and ExpiCHO™ enhancer (15 μL for 2.5 mL of culture volume; 2.4 mL for 400 mL of culture volume) were added to the cells. The cells were returned to INFORS HT incubator set at 37° C. under 8% CO₂ and 80% humidity with shaking at 125 rpm (25 mm orbit). 8 days post-transfection, supernatants were clarified by centrifugation at 4000×g for 30 min. Supernatants were filter-sterilized using a Nalgene™ Rapid-Flow™ Sterile Disposable Filter Units 1000 mL filter unit (Thermo Scientific, Cat. no. 567-0020) and were stored at 4° C. or frozen for later analysis.

Freshly thawed Expi293F cells were allowed to recover in culture for two or more passages before transfection. Cells were then passaged every 3-4 days until they reach 3×10⁶-5×10⁶ cells/mL at which time they were diluted to 3×10⁵-5×10⁵ cells/mL in Expi293™ Expression Medium pre-warmed to 37° C. The day prior to transfection, cells were diluted to 2.5×10⁶-3×10⁶ and on the day of transfection, cells were further diluted to 3×10⁶ viable cells/mL. 1 μg of DNA/mL of culture volume was diluted with Opti-MEM™ I Reduced Serum medium to get a final volume of 150 μL for 2.5 mL of culture volume and 24 mL for 400 mL of culture volume. ExpiFectamine™ 293 Reagent (8 μL for 2.5 mL of culture volume; 1.3 mL for 400 mL of culture volume) was added to medium Opti-MEM™ I Reduced Serum medium (140 μL for 2.5 mL of culture volume; 22.5 mL for 400 mL of culture volume) to incubate at room temperature for 5 minutes. Diluted ExpiFectamine™ was added to diluted DNA and incubate for 15 minutes at room temperature. ExpiFectamine™/DNA solution was transferred to culture drop by drop (at 3×10⁶ cells/mL) while swirling. The cells were incubated at 37° C. under 8% CO₂ and 80% humidity with overnight shaking (INFORS HT shaker, 125 rpm). 18-22 h after onset of transfection, ExpiFectamine™ 293 Transfection Enhancer 1 (15 μL for 2.5 mL of culture volume; 2.4 mL for 400 mL of culture volume) and ExpiFectamine™ 293 Transfection Enhancer 2 (50 μL for 2.5 mL of culture volume; 24 mL for 400 mL of culture volume) were added to the cells. The cells were returned to INFORS HT incubator set at 37° C. under 8% CO₂ and 80% humidity with shaking at 125 rpm (25 mm orbit). 5 days post-transfection, supernatants were clarified by centrifugation at 4000×g for 30 min. Supernatants were filter-sterilized using a Nalgene™ Rapid-Flow™ Sterile Disposable Filter Units 1000 mL filter unit (Thermo Scientific, Cat. no. 567-0020) and were stored at 4° C. or frozen for later analysis.

Proteins are purified using 3-mL MabSelect SuRe resin (GE Healthcare, Cat. No. 17-5438-02) with gravity columns or 40-mL MabSelect SuRe resin with AKTA PURE (GE Healthcare, Piscataway, NJ) depending on the supernatant volume. Resin was incubated with 0.5 NaOH for one hour and equilibrated with Tris-base buffer pH 7.4 (50 mM Tris-HCl, 150 mM NaCl, pH 7.4) prior injection. Supernatant was applied on gravity columns or the at 5 mL/min on 40-mL column. Resin column was washed with 3 CV (column volume) with Tris-base buffer pH 7.4 at flow rate of 10 mL/min. Protein was eluted with 3 CV of 0.1M citrate acid pH 3 at 10 mL/min. Fractions identified with protein from the visual output of the chromatogram (absorbance at 280 nm) were pooled together. Pooled fractions were neutralized with 1 M Tris-HCl pH 9 to achieve the pH~5-6 before transferring into PBS (Phosphate-buffered saline) pH 6 buffer prepared from PBS 10× pH 7.2 (15 mM Potassium Phosphate monobasic 1552 mM Sodium Chloride 27 mM Sodium Phosphate dibasic, ThermoFisher, Cat. no. 70013073) or 20 mM Histidine 7% Sucrose 0.02% Polysorbate80 pH 5.5.

Buffer exchange was carried out by sample concentrators for proteins purified from gravity columns or either by dialysis or by desalting column for proteins purified from AKTA PURE. Proteins purified from gravity columns were concentrated with sample concentrator VivaSpin 2, 50 kDa MWcO (GE Healthcare, Cat. no. 28932257) by centrifugation at 3,500-4,000×g at 4° C. then, diluted with PBS pH 6 to achieve 4-fold and repeated until sample reached 200-fold. Dialysis was carried out in 4 L of PBS pH 6 overnight at 4° C. using 7 kDa molecular weight cut-off dialysis tubing (ThermoFisher, Cat. no. 68799). On the other hand, desalting column was incubated with 0.5 NaOH overnight and equilibrated with PBS pH 6. Volume of 15 mL of neutralized protein sample was loaded into the HiPrep 26/10 desalting column (GE Healthcare, Cat. no. 17-5087-02) at 0.5 mL/min then, protein was eluted with 2 CV of PBS pH 6. Loading and elution steps were repeated until no neutralized protein sample from elution of affinity column remained. Fractions identified with protein from the visual output of the chromatogram (absorbance at 280 nm) were pooled together.

Sample was filter-sterilized using a Nalgene™ Rapid-Flow™ Sterile Disposable Filter Units 150 mL filter unit (Thermo Scientific, Cat. no. 565-0010). Final protein sample was quantified by optical density at 280 nanometer and tested for endotoxin level with Endosafe® LAL Reagent cartridges (Charles River Cat. no. PTS2005). Final protein sample was analyzed on SDS-PAGE gels under reducing or non-reducing conditions (see section SDS PAGE and Western Blotting).

### Example 3—In Vivo Anti-Tumor Effect

The anti-tumor effect of the sdAbs of the present disclosure was tested in established tumor models in mice.

NCG mice (5-6 weeks old, female) were purchased from Charles River Laboratories (St. Constant, QC). CB-17 Fox Chase SCID mice (6-8 weeks old, female) were purchased either from Charles River Laboratories (St. Constant, QC) or from UHN (Toronto, ON). Mice were housed in a pathogen-free environment at the animal facility in UHN. The animal work was conducted according to the guidelines of the Canadian Council on Animal Care (CCAC) and the Animal Use Protocol approved by the Animal Care Committee at UHN.

Briefly, SCID mice or NCG mice were implanted subcutaneously with 5 or 10 million tumor cells. Animals were randomized into experimental groups when tumor volumes reached 100 mm³ and were then treated (arrows) with the human IgG1 fusion construct of the KD001-KD005 or with PBS (negative control). Tumor volume was measured twice per week.

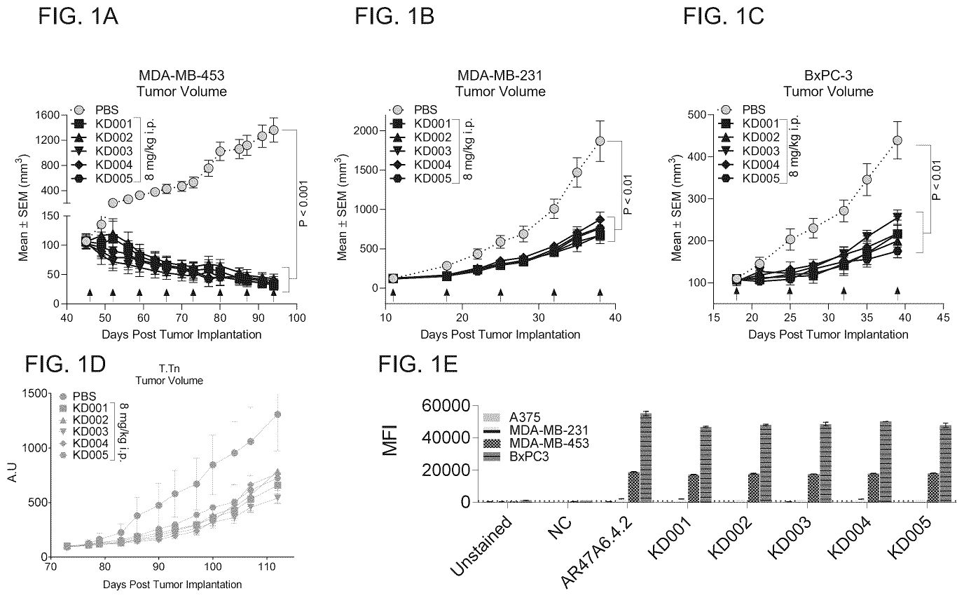

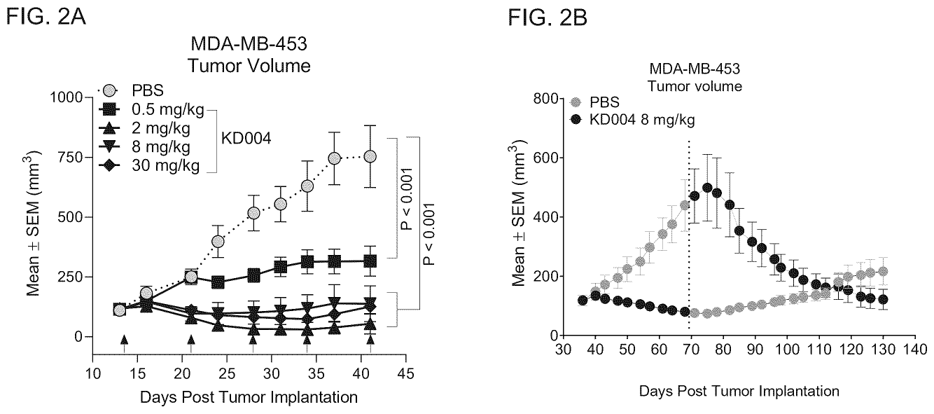

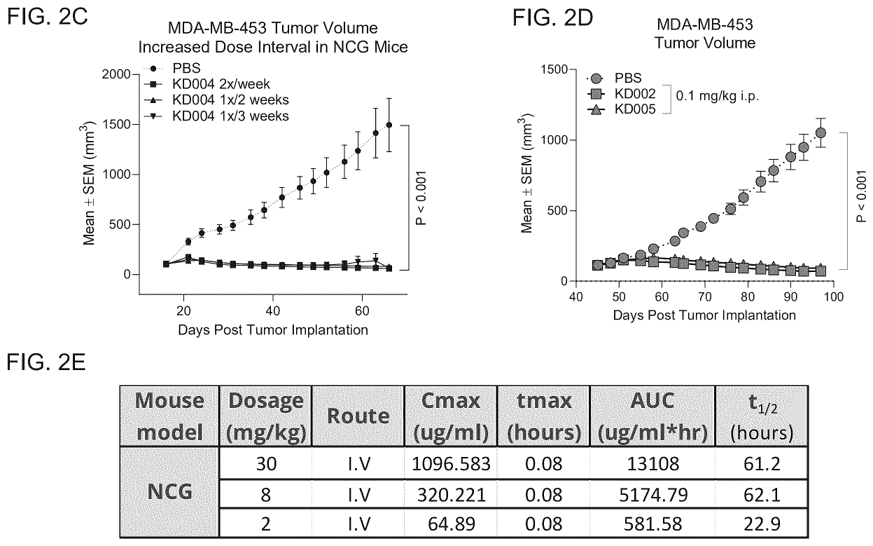

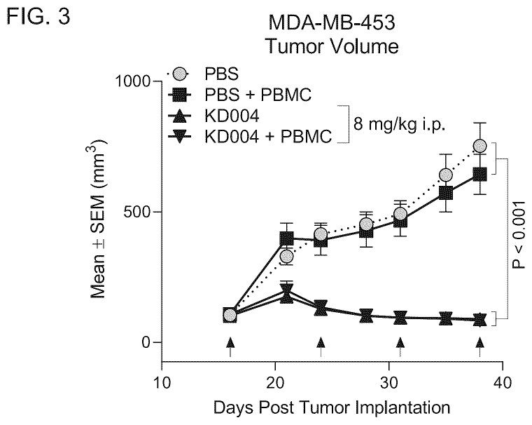

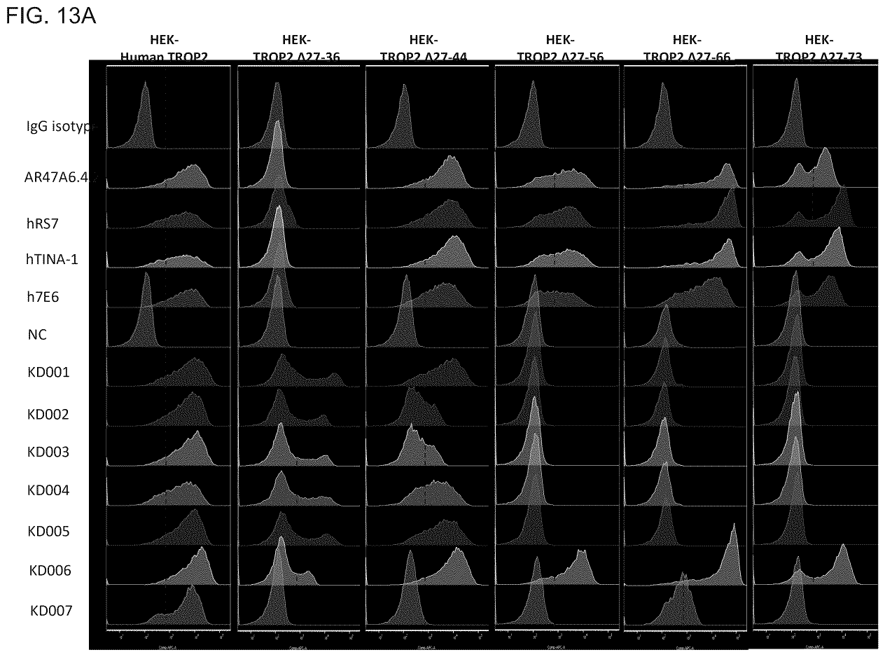

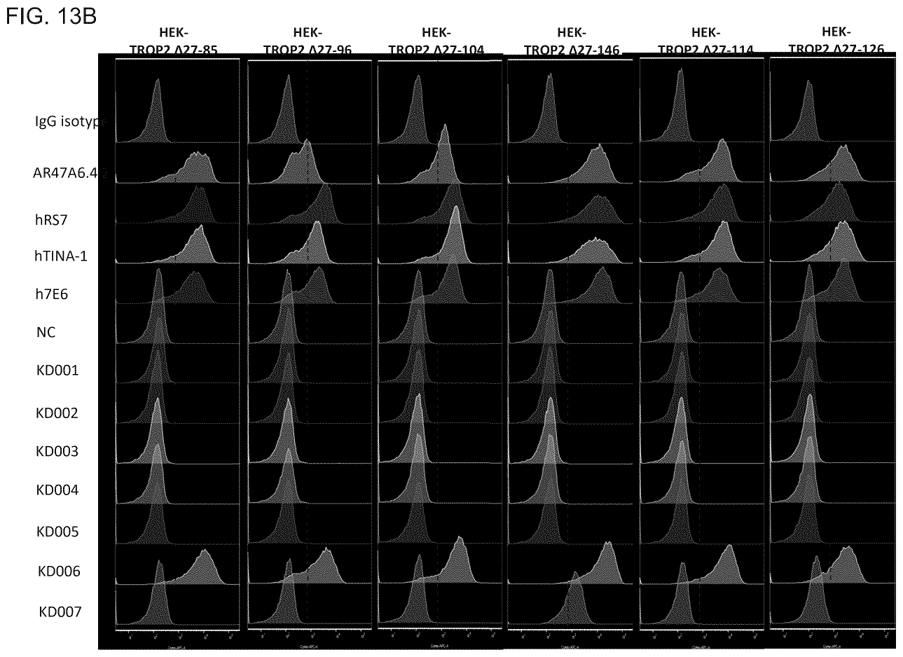

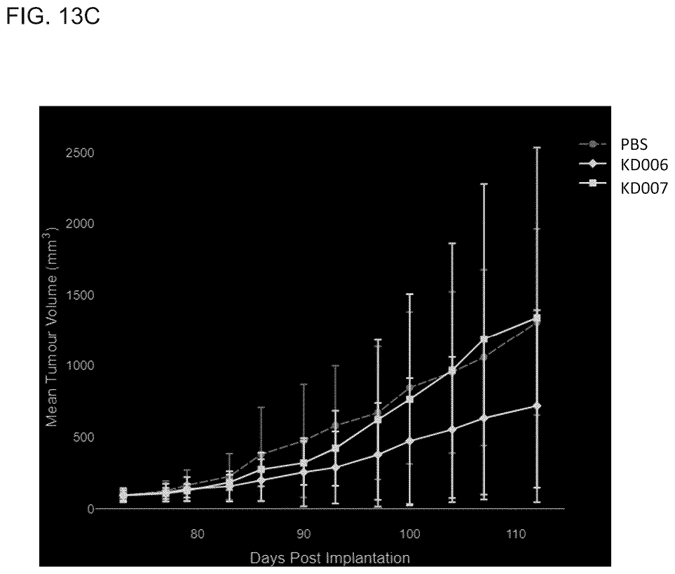

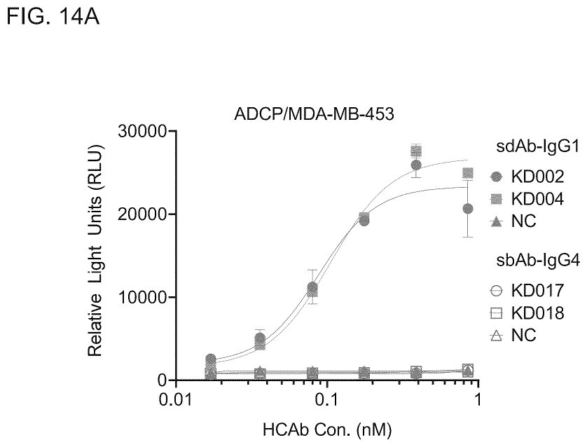

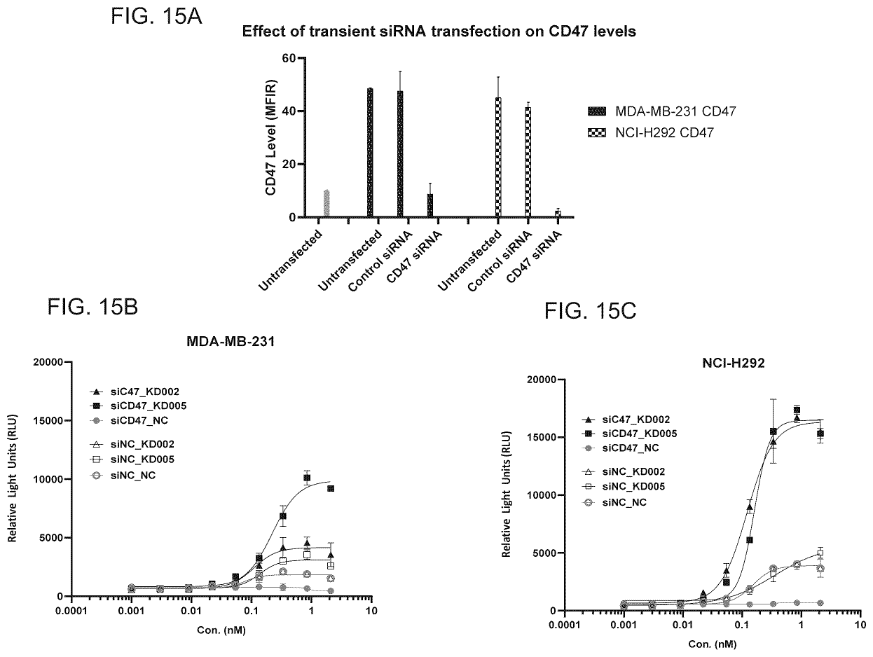

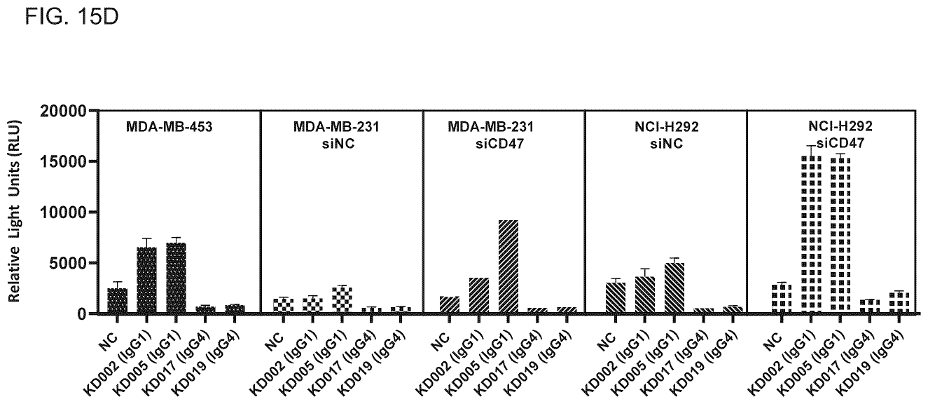

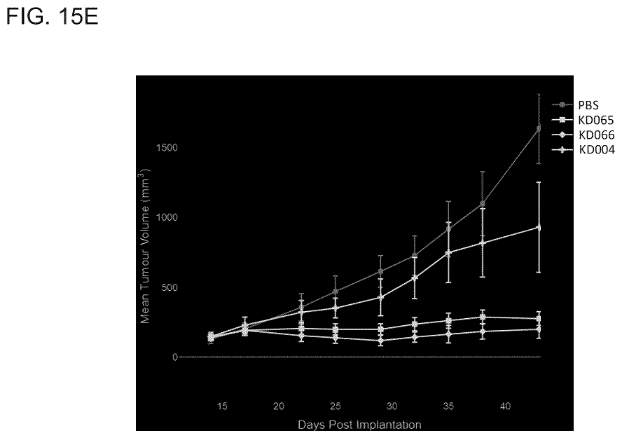

Antibody efficacy was tested in SCID or in NCG mice bearing MDA-MB-453, MDA-MB-231, BxPC-3, T.Tn tumors or MDA-MB-468 tumors. In FIG. 1A, the NCG mice were treated with testing articles at 8 mg/kg, once per week for 8 doses. In FIG. 1B to 1D, the SCID mice were treated with testing articles at 8 mg/kg, once per week for 8 doses. In FIG. 2A, the NCG mice were treated with testing articles at 0.5, 2, 8, 30 mg/kg once per week for 8 doses. The PBS control arm were treated with testing articles at 8 mg/kg in FIG. 2B. In FIG. 2C, the NCG mice were treated with KD004 at 8 mg/kg with different dosing frequencies, including twice per week, every two weeks, and every three weeks. In FIG. 2D, the SCID mice were treated with testing articles at 0.1 mg/kg once per week for 6 doses. In FIG. 3, the NCG mice were engrafted with or without 10 million PBMC and then treated with KD004 at 8 mg/kg, twice per week for 8 doses. In FIG. 13C, SCID mice were treated with testing articles at 8 mg/kg once per week for 6 doses. In FIG. 15E, SCID mice were treated with testing articles at 8 mg/kg once per week for 6 doses.

No signs of toxicity were observed throughout the studies. All sdAb-treated mice had significantly smaller tumors compared to the PBS-treated animals (FIGS. 1A-1D).

The binding of anti-TROP2 sdAbs to several cancer cell lines including MDA-MB-453 (triple negative breast cancer), MDA-MB-231 (triple negative breast cancer), BxPC3 (pancreatic cancer), and A375 (melanoma) cells were analyzed by flow cytometry (FIG. 1E). 1×10⁵ cells were incubated with an Fc receptor blocking antibody (1/100 dilution) for 10 minutes at room temperature. Anti-TROP2 antibodies were diluted in FACS staining buffer to 10 nM and added and incubated with cells for 30 minutes on ice. After washing three times with FACS staining buffer, FITC-conjugated secondary antibodies recognizing the human Fc region (Biolegend) were mixed with cells. After staining for 30 minutes, cells were washed three times, followed by resuspension into 100 μL of 7-Amino-Actinomycin D (7-AAD) solution (1/100 dilution). Cells were analyzed by BD FACSCanto™ II Flow cytometer (BD Bioscience). Data were analyzed with FlowJo via gating on single cells and live cells.

As illustrated in FIG. 1E, all the tested anti-TROP2 antibodies exhibited similar binding to tested cancer cell lines. All the tested anti-TROP2 antibodies showed the highest binding to BxPC3 cells, intermediate binding to MDA-MB-453 cells, and weak binding to MDA-MB-231 cells. None of the TROP2 antibodies bound to low TROP2 expression cell lines A375, illustrating the binding specificity of tested antibodies to TROP2. The binding of TROP2 sdAbs showed comparable binding to MDA-MB-231, MDA-MB453, and BxPC3 as AR47A6.4.2 TROP2 antibody (see Truong et al., 2008).

### Example 4—Dose-Response, Schedule, Regimen and Pharmacokinetics

In FIG. 2A and FIG. 2B, all the NCG mice were humanized with human PBMCs (10 million cells, i.v.) the day before treatment initiated, represented by the first time point in the graphs. In FIG. 2C and FIG. 2D, the NCG mice were not humanized with human PBMCs. Human PBMC cells (10 million cells/mouse were injected i.v.) were received from HemaCare Corporation (Northridge, CA). Tumor cells were implanted in NCG mice in a similar fashion to SCID mice. In all models, tumor volumes were measured using vernier calipers and the mice were weighed one or two times weekly. Tumor volume was calculated using the formula: ½ (Length×Width²).

As illustrated in FIG. 2A, the in vivo dose/response study indicates that weekly treatment of sdAbs KD004 (0.5 mg/kg, 2 mg/kg, 8 mg/kg and 30 mg/kg) prevents the growth of MDA-MB-453 implanted in NCG mice (5 million cells s.c.). No signs of toxicity were observed when animals were treated with higher doses.

Late intervention with anti-TROP2 therapy caused tumor regression in tumors of very large size (average 500 mm³) when dosed with KD004 at 8 mg/kg, once weekly (QW) in the MDA-MB-453 tumor model in NCG mice. (FIG. 2B).

Potent tumor regression was also achieved even when anti-TROP2 sdAb KD004 was administered once every three weeks in the MDA-MB-453 tumor model, in NCG mice, at a dose of 8 mg/kg (FIG. 2C).

Treatment with KD002 or KD005 at a dose of 0.1 mg/kg (i.p.) in MDA-MB-453 implanted NCG mice tumor model also led to tumor regression (FIG. 2D). The data indicated a strong anti-tumor activity of KD002 and KD005 in the pre-clinical MDA-MB-453 model. Such a strong activity may be explained by the higher binding affinity and potent ADCP activity mediated by macrophages.

Different groups of NCG mice were administered a single dose of KD004 at 2, 8 or 30 mg/kg dose level. Blood samples were collected at different time points and serum was prepared. Level of KD004 in serum was measured by ELISA and the pharmacokinetic parameters were calculated using the WinNonLin software. The anti-TROP2 in vivo pharmacokinetic parameters show that the half-life of KD004 is approximately 60 hours when administered at a dose of 8-30 mg/kg i.v. in NCG mice (FIG. 2E).

As illustrated in FIG. 3, the sdAb of the present disclosure possess anti-tumor activity as a naked antibody but may also induce ADCC.

The mechanism of action of the sdAbs of the present disclosure was further investigated.

### Example 5—Cell Viability

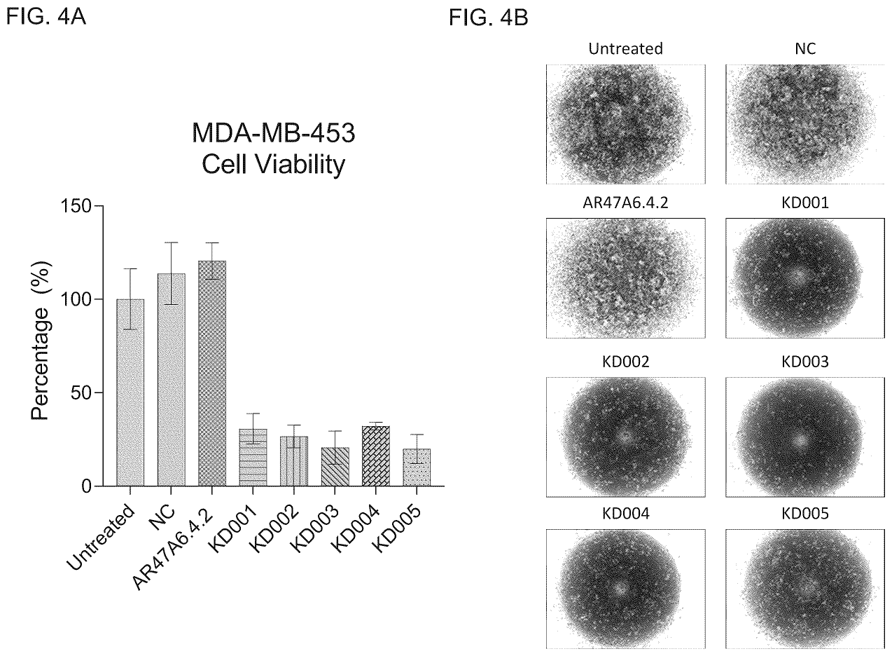

Cell viability of MDA-MB-453 cells following 12 days treatment with 1 μM TROP2 sdAbs was analyzed using the Promega CellTitre-Glo luminescent assay (FIGS. 4A-4B). FIG. 4A. MDA-MB-453 cells were seeded in 96-well plates at 5×10³ cells per well and incubated overnight at 37° C., 5% CO₂. Cells were treated with 1 μM TROP2 sdAbs for 12 days. Following treatment cell viability was assessed using CellTitre-Glo luminescent assay (Promega) according to manufacturer's instructions. Negative control wells were treated with isotype control sdAb (sdAb with variable region specific for HEWL). AR47A6.4.2 TROP2 antibody was used for comparison purposes. Control wells containing cells which did not receive any treatment were used for signal normalization and calculation of percentage cell viability following treatment. Background luminescence was determined using wells containing medium without cells. Values represent the mean±S.D. of three replicates for each treatment. FIG. 4B shows cells imaged at 4x resolution after 12 days of treatment with TROP2 sdAb. Images were captured using EVOS XL Core Imaging System.

As shown in FIGS. 4A-4B, the TROP2 sdAbs of the present disclosure (KD001, KD002, KD003, KD004 and KD005) directly inhibited MDA-MB-453 cell viability more than 50% compared to AR47A6.4.2 TROP2 antibody (which in turn did not directly affect cell viability) and the negative control (sdAb with variable region specific for HEWL).

### Example 6—ADCC Activity

The ADCC activity of sdAbs was further characterized using the Promega ADCC reporter assay kit (Promega, G7015). In brief, the target cells MDA-MB-453 cells were collected and using TrypLE and resuspended at the concentration of 3 million/mL. 12,500 cells were seeded into each well and incubated overnight. The next day, testing antibodies were diluted into the assay medium, with a final testing concentration of 1 μg/mL. The ADCC effector cells were thawed gently in a 37° C. water bath and resuspended into the assay medium. The ratio between effector cells and target cells used was 1:10. Thus 1,250 effector cells were transferred to each well. Plates were incubated at 37° C. incubator for 24 hours. 24 hours later, plates were equilibrated to room temperature. The Bio-Glo luciferase assay substrate was added to each well. Incubate the plate at room temperature for 5-30 minutes. Luminescence signal was measured by SpectraMax i3x plate reader. Data were analyzed by signal fold change upon untreated control wells.

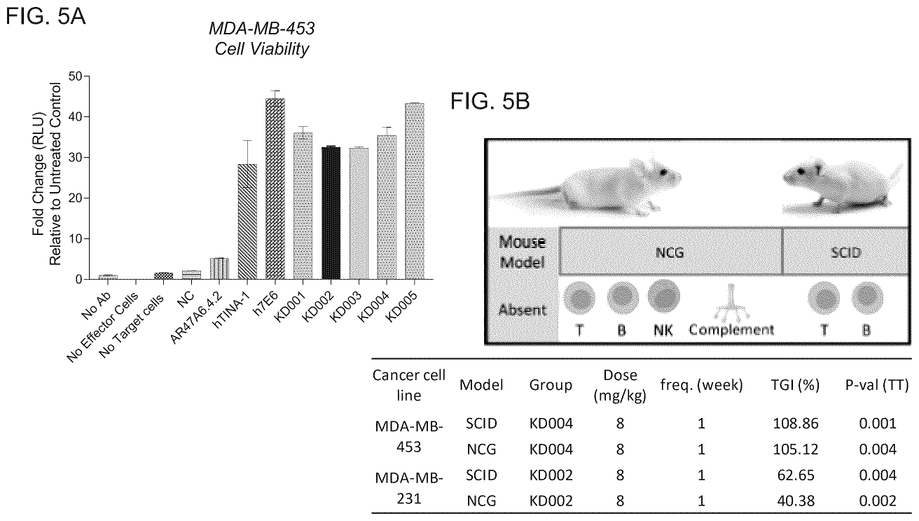

As shown in FIG. 5A, the tested anti-TROP2 sdAbs showed high ADCC capability against MDA-MB-453 cells. This ADCC reporter assay also indicates that KD001, KD002, KD003, KD004 or KD005 anti-TROP2 sdAbs are capable of inducing ADCC comparable to the human IgG1 anti-TROP2 antibodies generated with the sequences from clones hTINA-1 and h7E6.

NCG and SCID mice were implanted with either MDA-MB-231 or MDA-MB-453 cells s.c. Anti-TROP2 sdAb (KD002 or KD004) administration was initiated when the average tumor volume reached 100 mm³. As illustrated in FIG. 5B, sdAbs in vivo activity is similar in mouse strains with different effector function components.

### Example 7—Internalization

The internalization assay was divided into two steps. First, the anti-TROP2 sdAbs were conjugated with Promega pHAb Amine reactive dye (Promega, G9841) using on-bead antibody conjugation. Second, the internalization assay was carried out and analyzed by flow cytometry.

For pHAb amine-reactive dye labeling, anti-TROP2 sdAbs were conjugated To the protein A beads. anti-TROP2 sdAbs were mixed with protein A beads and incubated for 60 minutes at room temperature. The antibody/bead mix was then washed with the washing buffer and resuspended in the amine/thiol conjugation buffer. The pHAb amine-reactive dye was added into the tubes containing antibody/bead mix with a 20 molar excess of dye and incubated at room temperature for 60 minutes. Tubes were placed in the magnetic stand and supernatant was discarded. Beads were washed another two times with washing buffer before elution into the elution buffer. Eluted antibody was resuspended into the neutralization buffer from the kit.

The internalization assay was performed with MDA-MB-453 cells in a flow-based format. The MDA-MB-453 cells were collected and diluted to 300,000 cells/mL. 100 μL of cell solution (30,000) was transferred to the 96-well plates. Cells were incubated in the incubator overnight. The next day, pH conjugated antibodies were diluted to 10 μg/mL concentration. 50 μL of the pH conjugated antibodies were distributed into the corresponding wells and incubated to the indicated timepoints (4 hours and 24 hours). Plates were washed 1× with flow buffer (DPBS containing 2% serum) and cells were resuspended in 100 μL of flow buffer containing 7-AAD at 1:100 dilution. Plates were analyzed by BD FACSCanto II. Antibody internalized cells were gated based on 7-AAD negative and PE positive cells.

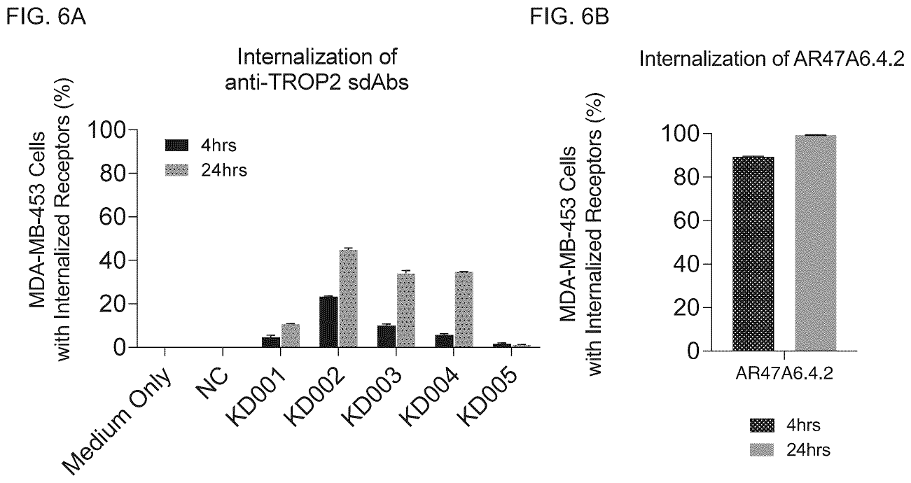

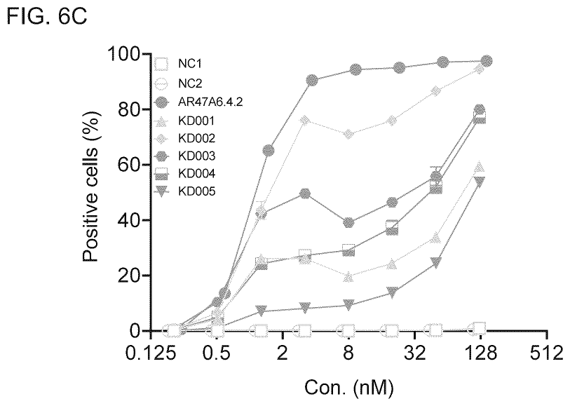

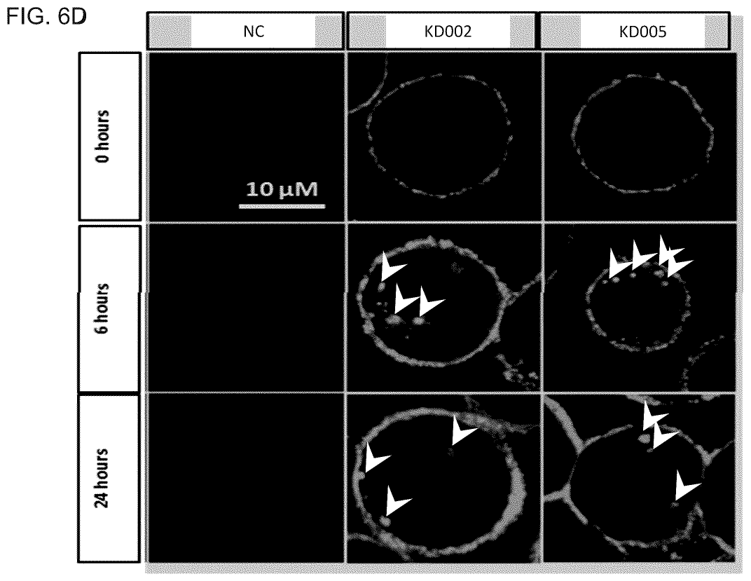

As illustrated in FIG. 6A and FIG. 6B, the positive control anti-TROP2 antibody AR47A6.4.2 showed internalization at 4 hours after incubation. The internalization of anti-TROP2 sdAbs was detected at 4 hours for KD002, KD003, and KD004. Increased internalization was observed at 24 hours for KD002, KD003, and KD004. KD002 showed the highest internalization among the anti-TROP2 sdAbs. Interestingly, KD005 was not internalized although it bound a similar epitope to the other anti-TROP2 sdAbs.

As illustrated in FIG. 6C, which represents the results from a dose-response internalization experiment in MDA-MB-231 cells, it was observed that each of the anti-TROP2 sdAbs KD001-KD005 (0.125 nM-128 nM range) display a unique dose-response internalization profile. Some sdAbs internalized only to a very small extent (e.g., KD005), whereas others showed internalization levels comparable to that of monoclonal antibodies known to internalize efficiently (e.g., KD002). Internalization was assessed by conjugation of sdAbs with a pH-sensitive probe and monitored by flow cytometry (Em. 560 nm) after 24 hours. This internalization process was also visualized by microscopy as illustrated by FIG. 6D. MDA-MB-453 cells were treated with αTROP2 sdAbs and internalization was monitored at 0 hours (after 15 minutes at 4° C.), 6 hours or 24 hours by confocal microscopy. Cells were stained with anti-human IgG Fc antibody and counterstained for nuclei (DAPI). Isotype negative control HCAb (NC) showed no binding to cells. Whereas anti-TROP2 sdAb staining is restricted to membrane at 0 h, and at 6 and 24 hours punctate green staining is observed (arrowheads) revealing antibody internalization.

These results indicate that the sdAbs of the present disclosure may be used as a naked antibody or as an antibody drug conjugate.

### Example 8—Specificity, Epitope Characterization and Domain Binding

The binding of anti-TROP2 sdAbs to cells was assessed by flow cytometry. To determine the binding specificity of anti-TROP2 sdAbs, the CHO and HEK parental cells were transiently transfected with human TROP2 by lipofectamine. 24 hours after transfection, cells were collected and stained with anti-TROP2 antibodies. In brief, 1×10⁵ cells were incubated with an Fc receptor blocking antibody (1/100 dilution) for 10 minutes at room temperature. Anti-TROP2 antibodies were diluted in FACS staining buffer to 100 nM and added and incubated with cells for 30 minutes on ice. After washing three times with FACS staining buffer, APC-conjugated secondary antibodies recognizing the human Fc region (Biolegend) were mixed with cells. After staining for 30 minutes, cells were washed three times, followed by resuspension into 100 μL of 7-Amino-Actinomycin D (7-AAD) solution (1/100 dilution). Cells were analyzed by BD FACSCanto™ II Flow cytometer (BD Bioscience). Data were analyzed with FlowJo via gating on single cells and live cells.

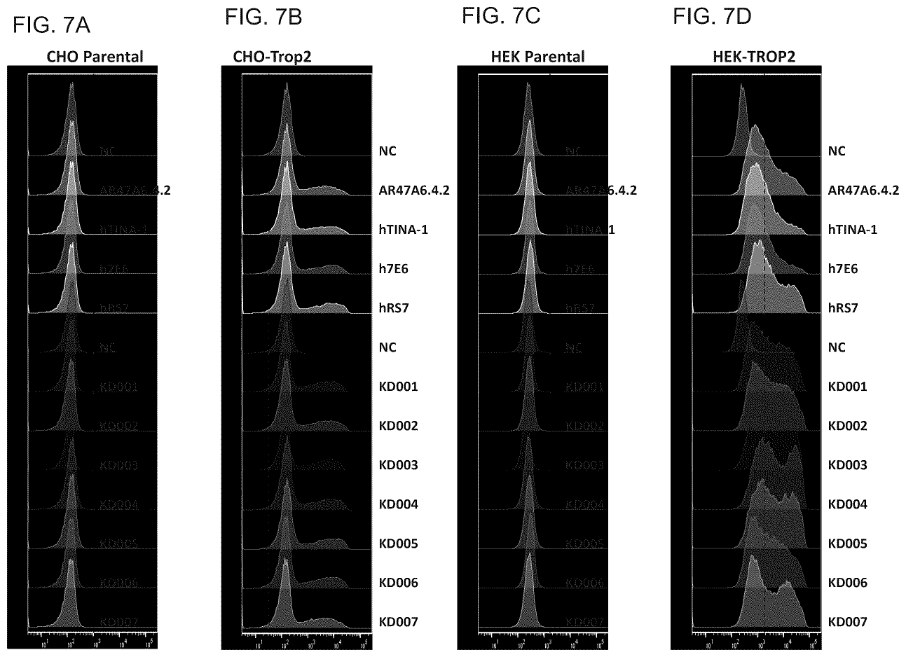

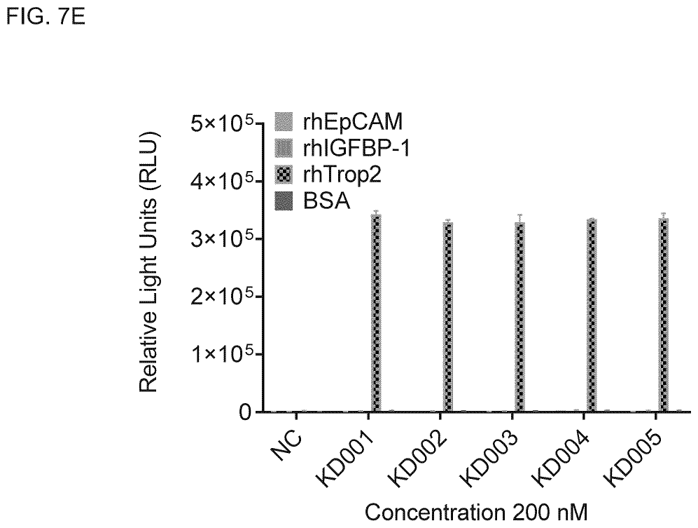

As illustrated in FIGS. 7A-7D, all anti-TROP2 antibodies did not bind to the HEK and CHO parental cells, which is similar to the negative control. After TROP2 transient transfection, all anti-TROP2 sdAbs showed specific binding to the HEK-TROP2 and CHO-TROP2 cells as also observed for comparator antibodies, AR47A6.4.2, hTINA-1, h7E6, hRS7 (see Cardillo et al. 2011).

Also as illustrated in FIG. 7E, binding of anti-TROP2 sdAbs is highly specific, as they did not bind to the highly related EpCAM and IGFBP-1 proteins, by ELISA.

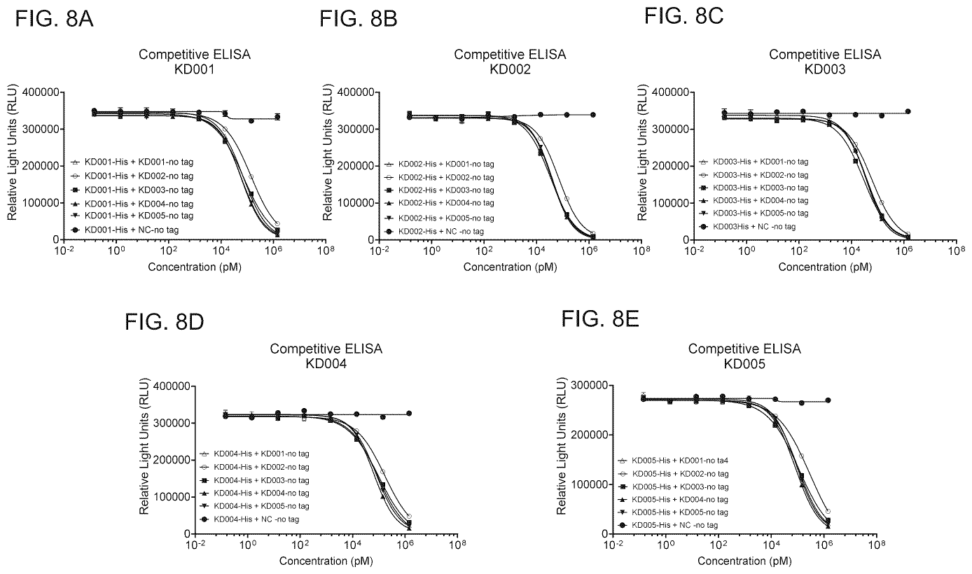

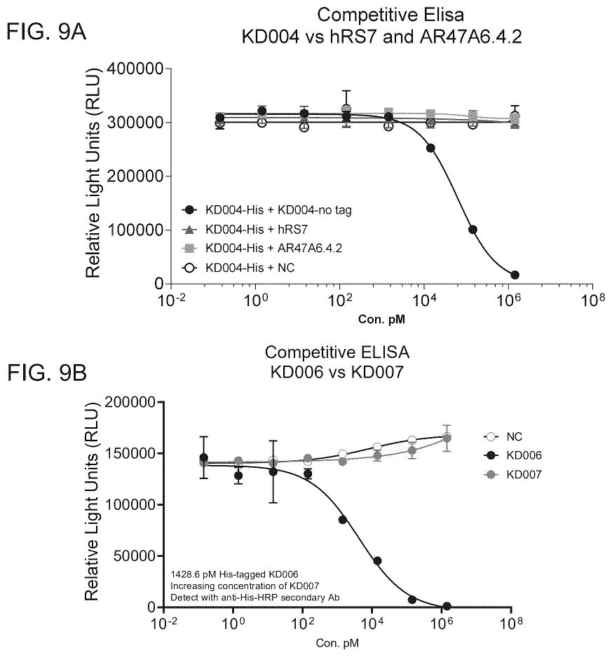

The epitope characterization was assessed by competitive ELISA (FIG. 8 and FIG. 9). Microplates were coated with human recombinant TROP2 at 4° C. overnight. After washing, plates were blocked with blocking buffer (PBS+2% BSA), for 1 hour at room temperature. Meanwhile, the dilution for anti-TROP2 sdAbs was prepared with blocking buffer making two different dilutions depending on the tag. For sdAbs with His tag, the antibody was diluted to 28571.4 μM. For sdAbs without tag, antibodies were serially diluted from 2857142.9 μM to 0.29 μM (8 dilutions, 10×). After that, equal volume of the his-tagged sdAbs and serial diluted non-tagged sdAbs were mixed, to make the final concentration of his-tagged sdAbs 14285.7 μM and non-tagged sdAbs from 1428571.4 μM to 0.14 μM. After washing two times, the mixed antibody contents were added into the wells and the plates were incubated for 2 hours at room temperature. The HRP-conjugated anti-6x His was diluted to 1:5000 and added to the wells after washing three times. Any unbound conjugate was removed by washing after 1 hour incubation at room temperature, and the colorimetric reaction was performed by adding the same volume of Supersignal ELISA Pico Luminol/Enhancer and SuperSignal ELISA Pico Stable Peroxide Solution and detected on SpectraMax i3x Microplate Reader. Positive and negative controls were included in each plate.

As illustrated in FIGS. 8A-8E, anti-TROP2 sdAbs KD001, KD002, KD003, KD004, and KD005 compete with each other for binding to human TROP2 recombinant protein, suggesting they bind to very similar epitopes binding of TROP2 or bind to overlapping epitopes.

Anti-TROP2 sdAb KD004 did not compete with hRS7 and AR47A6.4.2 for binding to human TROP2 recombinant protein, as illustrated in FIG. 9A, implying that KD004 binds different epitopes from hRS7 and AR47A6.4.2.

Anti-TROP2 sdAbs KD006 and KD007 did not compete for binding to human TROP2 recombinant protein (FIG. 9B), implying that KD006 binds a different epitope from KD007. KD006 and KD007 do not compete with KD001-KD005 in these experiments (data not shown).

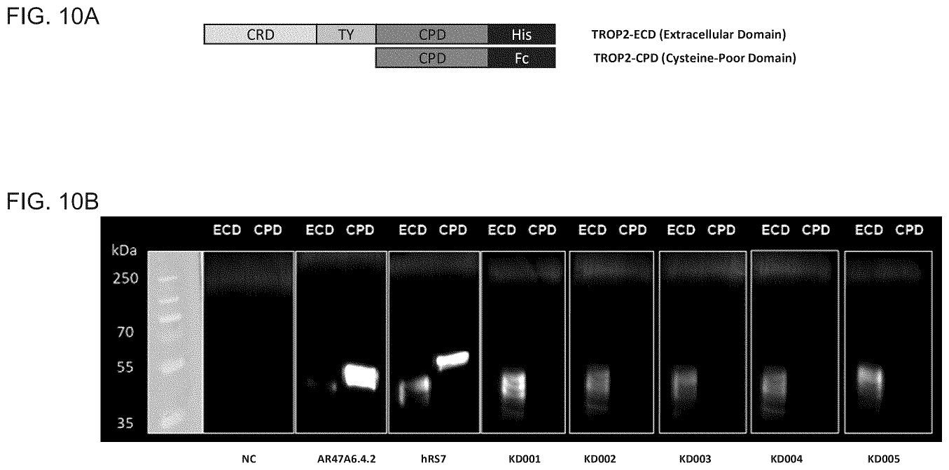

In order to decipher the domain specificity of TROP2 sdAbs, immunoblots were performed with various TROP2 constructs including the extracellular domain of TROP2 and the cysteine poor domain of TROP2 (FIG. 10A).

Recombinant protein of human TROP2 extracellular domain (ECD) (Sino Biological, Cat. No. 10428-H08H) was incubated with PNGase F enzyme (NEB, Cat. No. P0704) for the deglycosylation reaction under both denatured and non-denatured conditions as per the manufacturer's protocol. Samples were prepared for SDS-PAGE analysis under reduced conditions premixed with NuPAGE™ sample reducing agent (ThermoFisher Cat. No. NP0004) or non-reducing conditions without reducing agent.

Samples of recombinant proteins of human TROP2 extracellular domain (ECD) (Sino Biological, Cat. no. 10428-H08H) and human TROP2 cysteine poor domain (CPD) were prepared for SDS-PAGE analysis under reducing or non-reducing conditions with NuPAGE™ LDS Sample Buffer (ThermoFisher Cat. no NP0007) with NuPAGE™ Sample Reducing Agent (ThermoFisher Cat. no. NP0004) or without reducing agent. Samples under reducing condition were denatured by heating at 70° C. for 10 minutes. Samples (16 μL) were loaded onto 4-12% Bis-Tris mini-gels (1.5 mm, 15 wells) or 250 ng of samples were loaded onto 14% Tris-glycine mini-gels alongside a BSA standard. Electrophoresis was conducted using a X-Cell SureLock™ mini-gel device at 125 volts for approximately 1 hour. Gels were stained using GelCode™ staining reagent (Thermo Fisher, Cat. no. 24594).

Electrophoresis was conducted using a X-Cell SureLock™ mini-gel device at 125 volts for 1 hour. Proteins were transferred to nitrocellulose membranes using the iBlot™ system (Thermo Fisher, Cat. no. IB301031) according to the manufacturer's instructions. Membranes were blocked by incubation in PBS/0.1% Tween™ 20/5% Milk for 1 hour at room temperature with shaking, followed by incubation with 1 μg/mL of Anti-TROP2 antibodies or the isotype control (IgG1 isotype) in 20 mL of PBS/0.1% Tween™ 20/5% milk. Membranes were washed three times for 10 minutes with PBS/0.1% Tween™ 20. Membranes were incubated with HRP-conjugated goat anti-human IgG antibody (Sigma-Aldrich, AP113P) diluted at 1:10000 in blocking buffer for 1 hour at room temperature with shaking. Membranes were washed three times for 10 minutes with PBS/0.1% Tween™ 20. The signal was visualized using Super Signal™ West Pico PLuS (Thermo Fisher, Cat. no. 34080) according to the manufacturer's instructions. Images were captured using the Azure Biosystem imaging system.

As illustrated in FIG. 10B, all sdAbs tested bound to the extracellular domain of human TROP2 but did not bind to the cysteine poor domain of human TrOP2 contrary to other MAbs such as AR47A6.4.2 or hRS7.

The epitope recognized by the sdAbs of the present disclosure involves the CRD/TY-1 region of TROP2.

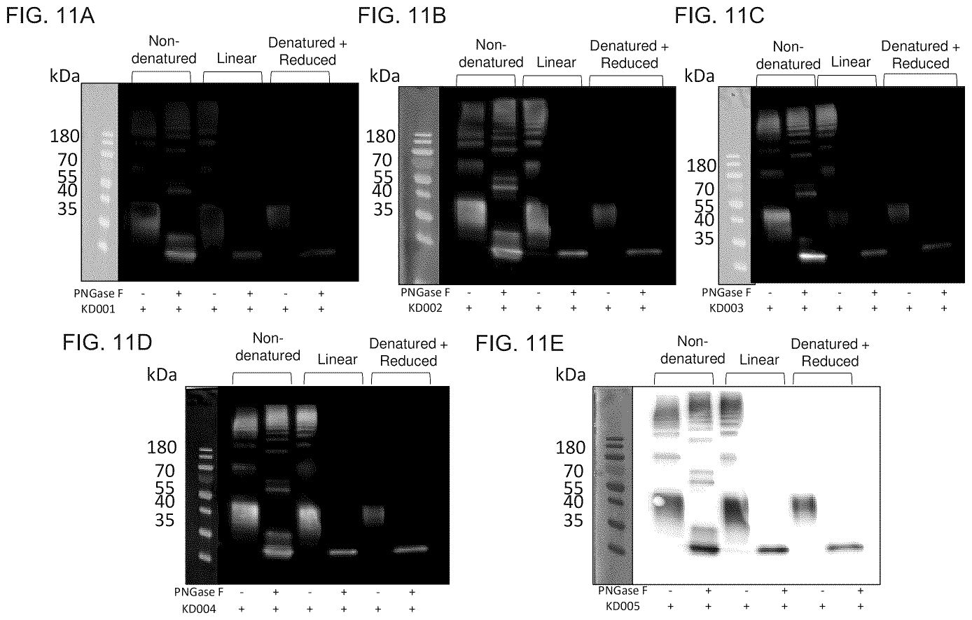

Western blots using recombinant human TROP2 in different forms (non-denatured, linear, reduced, deglycosylated using PNGase F) confirm that the sdAbs of the present disclosure effectively bind to TROP2 (FIGS. 11A-11E).

The specificity of sdAb for different TROP2 species was also investigated.

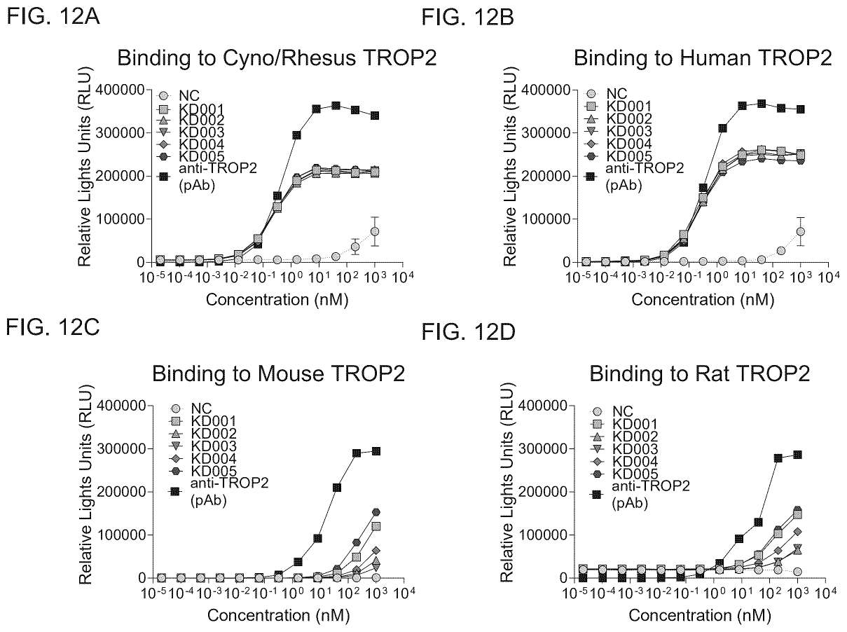

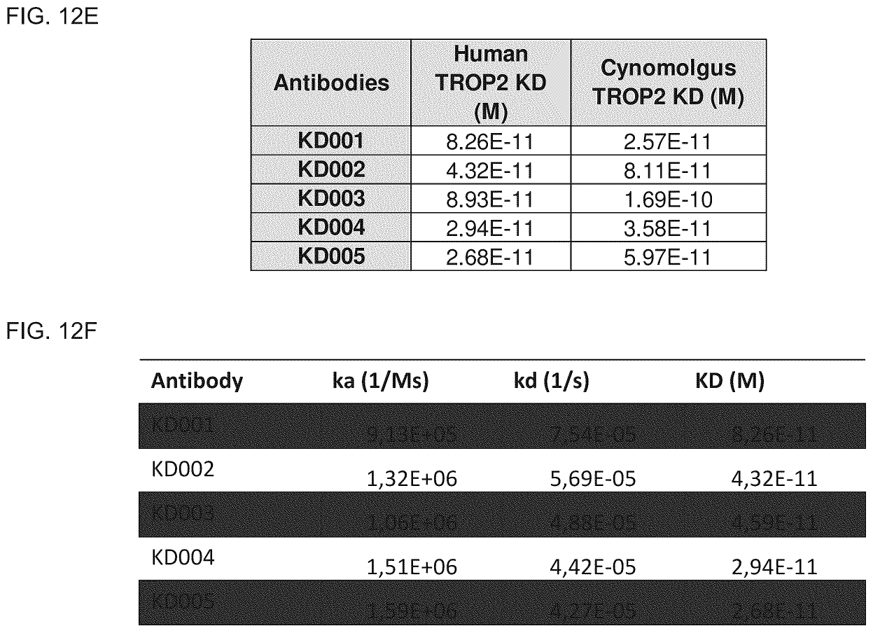

TROP2 full-length extracellular domain recombinant proteins including cynomolgus/Rhesus (FIG. 12A), human (FIG. 12B), mouse (FIG. 12C), and rat (FIG. 12D) were coated on 96-well ELISA plates at the concentration of 1 μg/mL. Plates were covered with adhesive plate cover and stored at 4° C. overnight. The next day, plates were washed 3x with washing buffer (PBS with 0.1% Tween™ 20). Plates were blocked with 2% fat-free BSA in washing buffer for 1 hour at 37° C. During the incubation time, the anti-TROP2 sdAbs were diluted in the blocking buffer to the indicated concentrations. In brief, the anti-TROP2 sdAbs starting concentration was 1000 nM and serial diluted 1 in 5 to 0.02048 μM. After blocking, the blocking buffer was removed from the wells and 100 μL of anti-TROP2 sdAbs dilutions were transferred to the corresponding wells. Plates were covered with adhesive plates cover and incubated at 37° C. for 1 hour. During the primary antibody incubation, the secondary antibody (goat anti-human IgG, HRP conjugated) was diluted 1 in 5000 with the blocking buffer. Plates were washed 3x with blocking buffer, followed by secondary antibody incubation for another 1 hour at 37° C. After the secondary antibody incubation, plates were washed 3x with washing buffer and added with 100 μL of ELISA chemiluminescent substrate (Thermofisher). The luminescence signal was detected by SpectraMax i3x at 425 nM.

As shown in FIGS. 12A-12D, the human anti-TROP2 sdAbs showed cross-reactivity to the cynomolgus/Rhesus TROP2, and a lower binding activity to mouse and rat TROP2 by ELISA.

All anti-TROP2 sdAbs have very high affinity, as shown by surface plasmon resonance (SPR) assay, both for human and cynomolgus TROP2 (low pM) (FIG. 12E). All anti-TROP2 sdAbs showed a very slow dissociation rate as indicated by the kd rate (FIG. 12F).

In order to determine the region of TROP2 targeted by the anti-TROP2 antibodies, the binding of anti-TROP2 antibodies to cells expressing TROP2 truncation was assessed by flow cytometry.

To determine the effect of TROP2 truncations on the binding ability of anti-TROP2 sdAbs, the HEK parental cells were transiently transfected with human TROP2 mutants by lipofectamine. 24 hours after transfection, cells were collected and stained with anti-TROP2 antibodies (FIG. 13A and FIG. 13B). In brief, 1×10⁵ cells were incubated with an Fc receptor blocking antibody (1/100 dilution) for 10 minutes at room temperature. Anti-TROP2 antibodies were diluted in FACS staining buffer to 100 nM and added and incubated with cells for 30 minutes on ice. After washing three times with FACS staining buffer, APC-conjugated secondary antibodies recognizing the human Fc region (Biolegend, Cat #410712) were mixed with cells. After staining for 30 minutes, cells were washed three times, followed by resuspension into 100 μL of 7-Amino-Actinomycin D (7-AAD) solution (BD Biosciences, Cat #559925).

As illustrated in FIG. 13A and FIG. 13B, clone AR47A6.4.2, hRS7, hTINA-1, h7E6 and KD006 binding to TROP2 was not affected by TROP2 truncation in the cysteine-rich domain. KD001, KD002, KD003, KD004, and KD005 lost their binding to TROP2 on HEK cells when TROP2 27-56 regions were truncated, implying that KD001-KD005 binding to TROP2 involves the TROP2 27-56 region.

Different from KD001 to KD005, KD006 and KD007 bound to another region to TROP2 proteins. Similar to other benchmark TROP2 antibodies, KD006 was able to bind to all TROP2 CRD truncations, implying that binding of these antibodies to TROP2 involves the TY-1 and/or CPD region. KD007 did not bind to any of the TROP2 CRD truncations, indicating that KD007 binding to TROP2 may involve TROP2 N-terminal 1-27 amino acids.

Treatment with KD006 led to a tumor growth inhibition of 48.6% in the xenograft model of T.Tn (esophageal carcinoma) whereas KD007 did not show efficacy in this model (FIG. 13C).

### Example 9—ADCP

The ADCP assays were performed with the Promega ADCP kits (cat #CS314906). In brief, 200,000 target cells, including MDA-MB-453, MDA-MB-231, NCI-H292, MDA-MB-468, T.Tn tumor cells were seeded into the 96-well plates overnight. The next day, testing antibodies were serial diluted (1 in 2 dilutions starting from 2 nM) into the RPMI 1640 culture medium. 25 μL of the serial diluted testing antibody was added to the wells containing appropriate adherent target cells.

THP-1 cells (ADCP Reporter Bioassay Effector Cells) were taken from −140° C. storage and transferred to the bench on dry ice. The effector cells were thawed in a 37° C. water bath until cells are just thawed (about 2-3 minutes). While thawing, the vial was gently agitated and visually inspected. The cell suspension was mixed in the vial by pipetting up and down and 0.5 ml cells was transferred to the tube containing 4.5 ml assay buffer. The tube was mixed by gently inverting. The ADCP Reporter Bioassay Effector Cells (THP-1) suspension were transferred to a sterile reagent reservoir and 25 μl of the cell suspension was dispensed to all the wells containing target cells and antibodies. The lid was placed on the plates and the plate was incubated for 4 hours at 37° C. in a CO2 incubator. 4 hours later, the Bio-Glo-NL™ Luciferase assay reagent was freshly prepared and reconstituted and equilibrated at room temperature. Using a manual multichannel pipette, 75 μl of io-Glo-NL™ Luciferase assay reagent was added to the assay plates while avoiding creating any bubbles. The plate was incubated at ambient temperature for 5-30 minutes and then luminescence was measured using a plate reader with glow-type luminescence read capabilities.

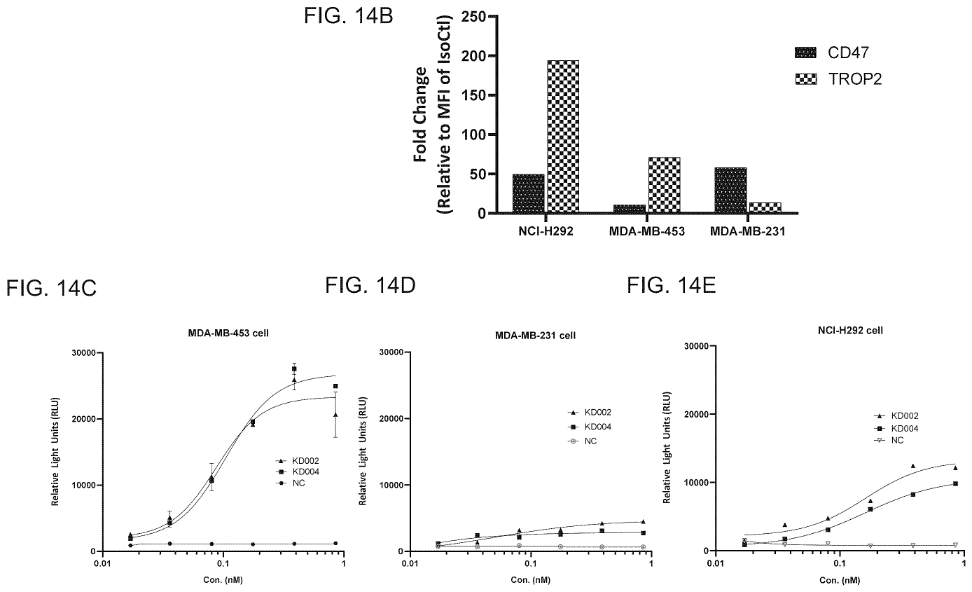

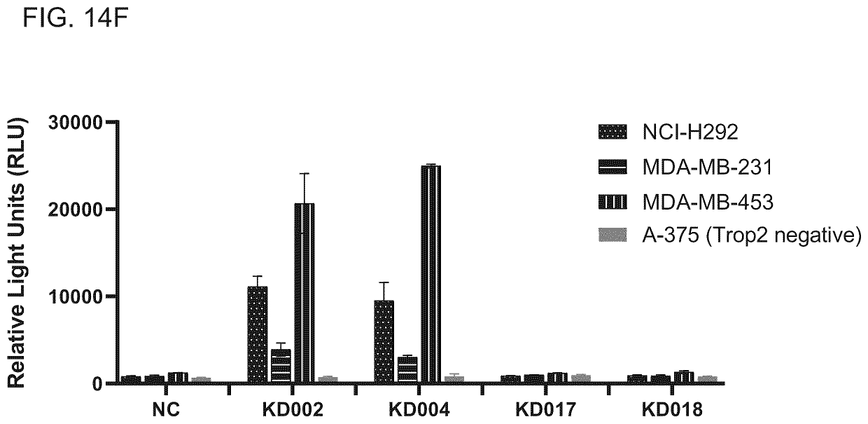

Experiments with KD002 and KD004 as well as corresponding versions in which the IgG1 Fc portion of KD002 and KD004 (KD017 and KD018) was replaced for an IgG4 Fc, were tested for antibody-dependent cellular phagocytosis (ADCP). As illustrated in FIG. 14A, the anti-TROP2 sdAbs IgG1 but not the IgG4 version elicited ADCP in vitro in a dose-response test (0.01 nM to 1 nM range). Antibodies' stock (20 nM) was serially diluted and added to the wells containing target and effector reporter THP-1 cells. MDA-MB-453 cells (target) were seeded at 20,000 cell/well overnight. The ADCP effector cells (THP-1 cells) were dispensed at an E:T ratio of 3:2.

Since CD47 is a checkpoint for macrophage ADCP activity, we assessed the level of CD47 expression in various cancer cells lines expressing TROP2 to evaluate the impact on the efficacy of anti-TROP2 sdAbs.

As illustrated in FIG. 14B, MDA-MB-453 cells showed the lowest CD47 expression, whereas the level of cell surface CD47 expression in NCI-H292 and MDA-MB-231 is similar. The level of TROP2 expression is higher in NCI-H292, followed by MDA-MB-453 and MDA-MB-231.

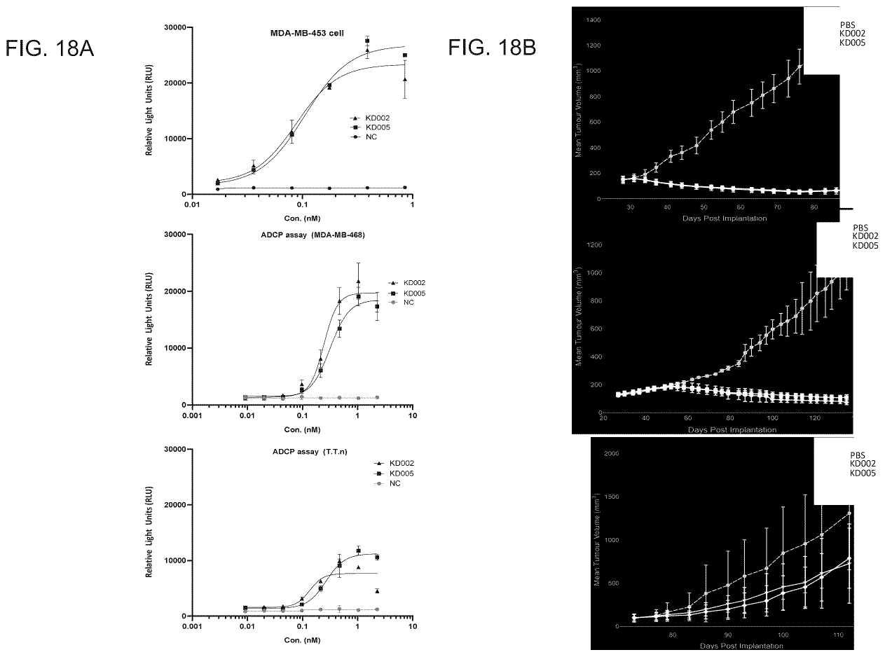

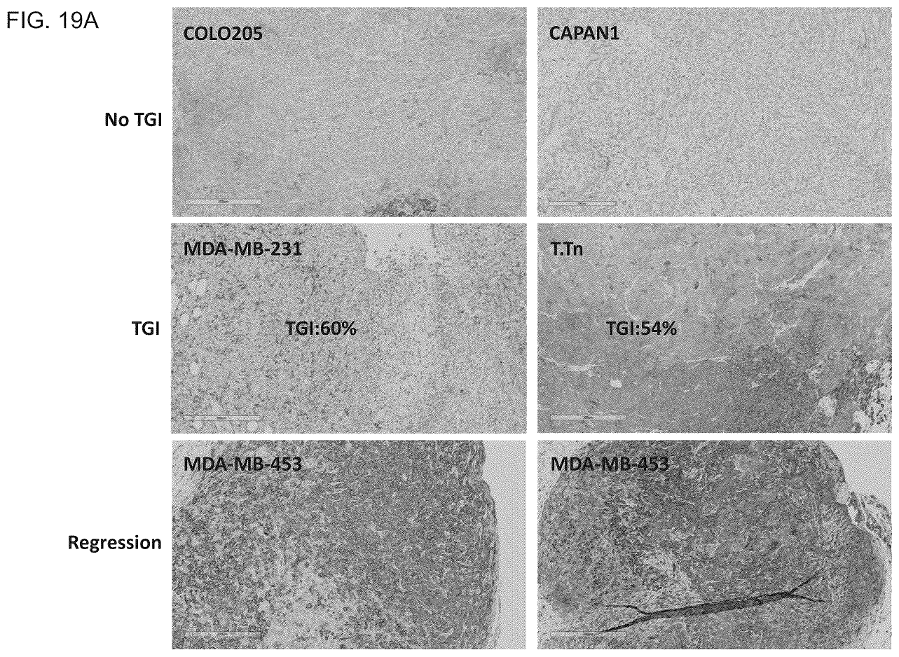

The ADCP assays were performed with the Promega ADCP kits (cat #CS314906). 200,000 target cells, including MDA-MB-453 (FIG. 14C and FIG. 18A-upper panel), MDA-MB-231 (FIG. 14D), NCI-H292 (FIG. 14E), MDA-MB-468 (FIG. 18A-middle panel) or T.Tn tumor cells (FIG. 18A-lower panel) were seeded into the 96-well plates overnight. The next day, testing antibodies (KD002 or KD004) were serial diluted (1 in 2.2 dilutions starting from 2 nM) into RPMI 1640 culture medium. 25 μL of the serial diluted testing antibody was added to the wells containing appropriate adherent target cells.

As illustrated in FIG. 14C-14E, TROP2 antibodies KD002 and KD004 showed a high level of ADCP activity against MDA-MB-453 cells and lower ADCP activity on MDA-MB-231 and NCI-H292 cells.

As shown in the FIG. 14C, the EC50 of the two antibodies KD002 and KD004 are close to each other at around 0.1 nM, but MDA-MB-453 there was a higher RLUmax than the MDA-MB-231 and NCI-H292 cells, which may be explained by the lower expression of CD47 on MDA-MB-453 cells.

As shown in FIG. 14F, IgG4 versions of the KD002 and KD004 antibodies (KD017 and KD018) have lost ADCP activity against MDA-MB-453, MDA-MB-231 and NCI-H292 cells in a similar experiment. These in vitro data suggest the ADCP mechanism of the TROP2 antibodies may be dependent on the Fc receptor.

Expression of CD47 was knocked down using small interfering RNA (siRNA) and similar ADCP assay were performed. MDA-MB-231 cells and NIC-H292 cells were transfected with CD47 siRNA (Thermofisher, Cat #4392421) or control siRNA (Thermofisher, Cat #AM4635) by the lipofectamine transfection kits. 48 hours after transfection, cells were collected and CD47 expression level was determined by flow cytometry. Once CD47 knockdown was confirmed, cells were used as the target cells in ADCP assays.

Expression level of CD47 and TROP2 on NCI-H292, MDA-MB-453 and MDA-MB-231 cells after CD47 knockdown was determined (FIG. 15A). 1×10⁵ cells were incubated with an Fc receptor blocking antibody (1/100 dilution) for 10 minutes at room temperature. Diluted APC-conjugated anti-CD47, PE-conjugated anti-TROP2 antibodies (Biolegend) (1/100 dilution) were mixed with cells. After staining for 30 minutes, cells were washed three times, followed by resuspension into 100 μL of 7-Amino-Actinomycin D (7-AAD) solution (1/100 dilution).

Cells were analyzed by BD FACSCanto™ II Flow cytometer (BD Bioscience). Data were analyzed with FlowJo via gating on single cells and live cells.

As illustrated in FIG. 15A, transfection with CD47 siRNA led to a significant reduction of CD47 expression in MDA-MB-231 and NCI-H292 cells.

As illustrated in FIGS. 15B-15D, CD47 knockdown by siRNA increased ADCP activity (phagocytosis) of KD002 and KD005 in both MDA-MB-231 and NCI-H292 cells. In MDA-MB-231 KD005 appears to have higher ADCP activity than KD002 and also show ADCP activity even without inhibition of CD47.

Moreover, as illustrated in FIG. 15D, in comparison to KD002 and KD005, the IgG4 versions of KD002 and KD005 antibodies (KD017 and KD019) have lost ADCP activity against MDA-MB-231 and NCI-H292 tumor cells even when CD47 expression is inhibited.

These results indicated that knockdown of CD47 significantly enhanced the maximal signal of the ADCP assay. Since CD47 is a checkpoint for macrophage ADCP activity, the higher expression of CD47 on MDA-MB-231 and NCI-H292 may explain the relatively low ADCP activity. This is consistent with data showing that higher CD47 expression led to the lower ADCP activity.

The in vivo ADCP mechanism and the potential enhancing ADCP activity was also verified with an anti-CD47 antibody. Briefly two bispecific antibodies comprising either KD011 at the N-terminus and KD070 at the C-terminus of Fc or KD011 at the C-terminus and KD070 at the N-terminus of Fc were generated. Both KD065 and KD066 showed increased anti-tumor activity compared to KD004 that does not include the anti-CD47 module (FIG. 15E).

These data suggest that potency of anti-TROP2 antibodies may be increased by inhibiting CD47. Bispecific or multispecific antibodies comprising an anti-TROP2 antigen binding domain and a CD47 antigen binding domain may therefore be used for treating cancer associated with TROP2 expression.

### THP-1 Differentiation

THP-1 cells were differentiated into macrophages in the six-well plates. Two million cells were plated in the six-well plates and stimulated with PMA (Sigma, P8139-1MG) at the concentration of 150 nM for 24 hours. Cells were washed with PBS and treated with cytokines for 72 hours in the cell culture medium. For M1 cell differentiation, cells were treated with 20 ng/ml IFN-gamma (Cedarlane, 285-IF-100) and 10 μg/ml LPS (Cedarlane, 437628-1MG). For M2 cell differentiation, cells were treated with 20 ng/ml IL-4 (Cedarlane, 204-IL-010) and 20 ng/ml IL-13 (Cedarlane, 213-ILB-010).

Before running macrophage phagocytosis assay, cells were collected with TrypLE and washed twice with PBS. The target cells MDA-MB-453 were stained with pHrodo SE (Invitrogen, P36600) at 120 ng/ml for 30 minutes at room temperature. The M1 or M2 macrophages were stained with Celltrace Violet (Invitrogen, C34557 A) at 5 μM for 30 minutes at room temperature. Cells. Cells were washed with PBS and resuspended in the RPMI complete medium. M1 or M2 macrophages were mixed with MDA-MB-453 cells at the E:T ratio of 2:1 and treated with the indicated antibodies for 4 hours. After the incubation, cells were washed with PBS and resuspended in the FACS buffer with 1/100 dilution of 7-AAD. Cells were analyzed by the FACS CantoII cytometer and double positive cells were gated as the phagocytosed macrophages.

FIG. 16A shows that THP-1 cells differentiate into M1 and M2 macrophages upon treatment with PMA and cytokines. THP-1 cells showed upregulation of CD68 after PMA treatment. The M1 cells showed upregulation of CD38 and CD86 after treatment with IFN-7 and LPS. M2 macrophages showed upregulation of CD206 and downregulation of CD38 after treatment with IL-3 and IL-13.

As illustrated in FIG. 16B, both M1 and M2 macrophages lead to phagocytosis of target cells MDA-MB-453 by KD002 and KD005.

### Example 10—In Vivo Mechanism of Action

The mechanism of action was further investigated in vivo.

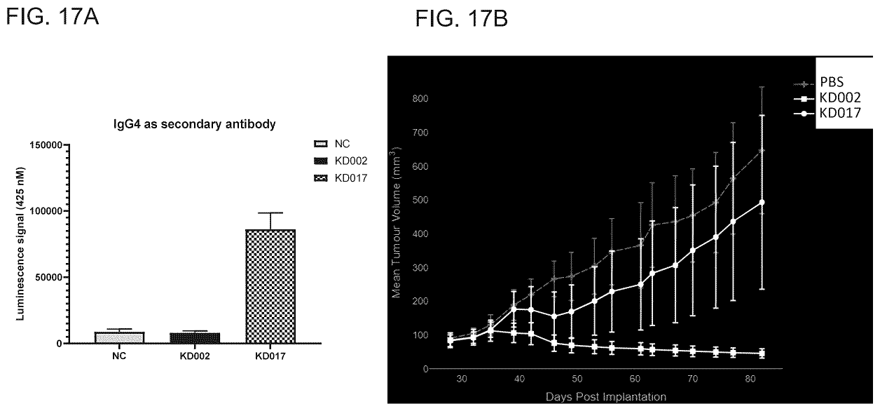

As illustrated in FIG. 17A KD017 which is the IgG4 version of KD002 was detected by the secondary antibody while as expected, the IgG1 version KD002 was not. KD017 is thus able to bind to recombinant human TROP2. However, as illustrated in FIG. 17B, KD017 has lost the anti-tumor effect in the MDA-MB-453 xenograft tumor model. These data suggest that KD002 anti-tumor function is a Fc receptor-dependent activity. Since the NCG mice only has myeloid cells but lack all B cell, T cell and NK cells, this Fc-dependent cellular activity is likely mediated by macrophage ADCP.

As illustrated in FIG. 18A, TROP2 antibodies KD002 and KD005 showed a high level of ADCP activity against MDA-MB-453 cells (upper panel) and MDA-MB-468 cells (middle panel) and a medium level of ADCP activity against T.T.n cells (lower panel). Corresponding to the higher in vitro ADCP activity, KD002 and KD005 treatment led to complete tumor regression in the MDA-MB-453 (upper panel), and MDA-MB-468 (middle panel) xenograft model (FIG. 18B). KD002 and KD005 led to a partial response in T.Tn xenograft tumor model with a tumor growth inhibition of 60% (FIG. 18B-lower panel).

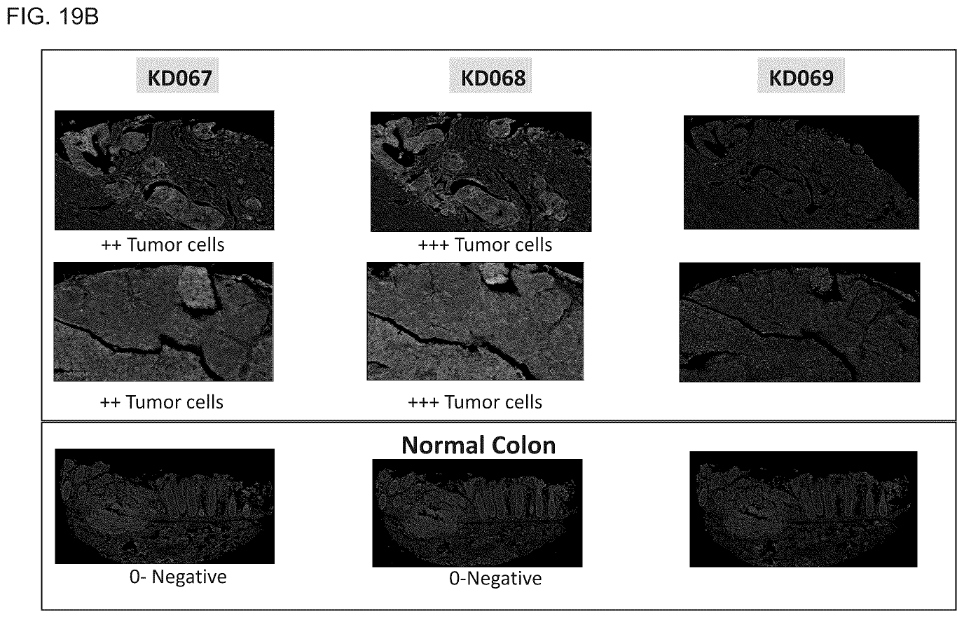

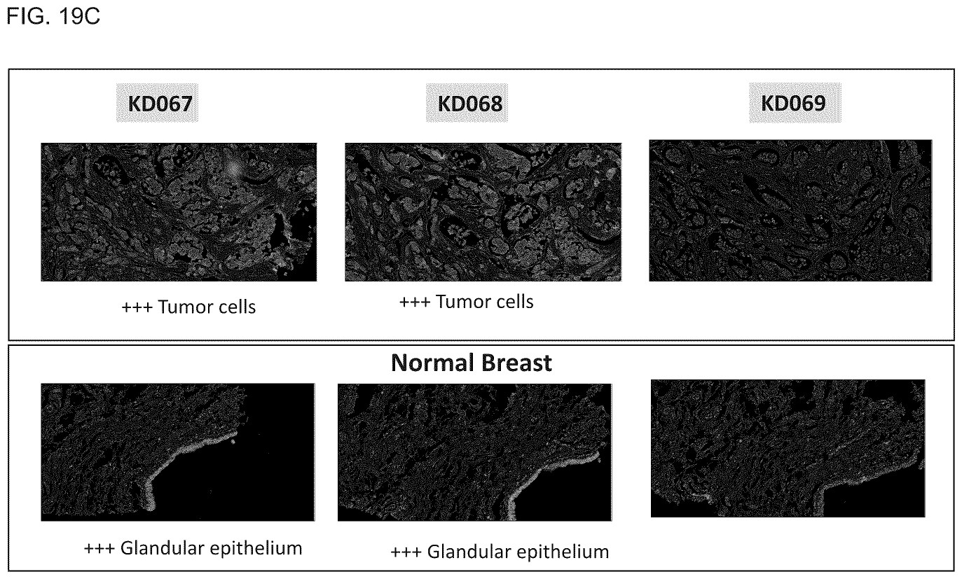

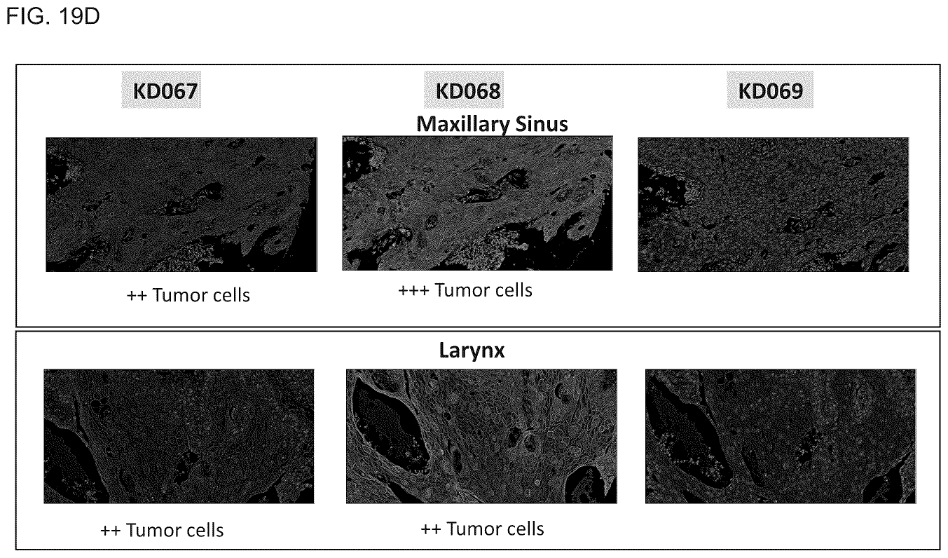

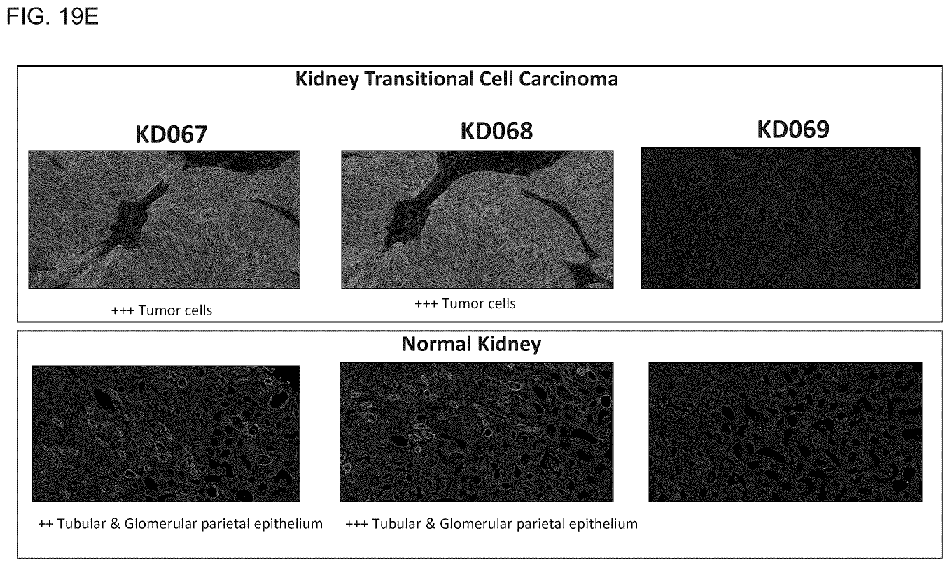

For mouse macrophage IHC staining, tumor samples including COLO205, CAPAN1, MDA-MB-231, T.Tn and MDA-MB-453 were collected from the xenograft models and formalin-fixed paraffin-embedded. Samples were stained with the mouse macrophage marker F4/80 followed by the secondary antibody-HRP (FIG. 19A). There was a correlation between the degree of macrophage infiltration and the potency of TROP2 antibodies. In the MDA-MB-453 xenograft model in which TROP2 antibodies treatment led to a complete regression, macrophage showed massive infiltration into the tumors confirmed by IHC. In the MDA-MB-231 and T.Tn xenograft model in which TROP2 antibodies showed a 50%-60% tumor growth inhibition, macrophage exhibited a medium level of macrophage infiltration. COLO205 and CAPAN1 xenograft model showed little macrophage infiltration and no tumor growth inhibition (TGI).

CD47 showed a higher expression in most of the tumors confirmed by IHC. However, overall, we observed a correlation between TROP2 antibodies efficacy and macrophage infiltration (FIG. 19A).

These data indicate that the TROP2 antibody potency involves macrophage infiltration into the tumors. TROP2 expression and macrophage infiltration could represent clinical biomarkers to achieve optimal treatment result.

### Example 11—TROP2 Expression in Selected Tumors

### TROP2 IHC Staining

To understand the TROP2 expression prevalence across different cancer types, we stained TROP2 expression with TROP2 antibodies at 2 mg/ml on the FFPE cancer tissue microarrays (Novus Biologics, cat #NBP2-42052 & NBP2-42056) and frozen colon cancer microarray (Biochain, T6235090) (FIGS. 19B-19E). In the Novus Biologics FFPE cancer tissue microarrays, TROP2 antibody showed high positive staining on ovary cancer (71%), bladder cancer (100%), breast cancer (100%), uterus cancer (83.3%), head and neck cancer (78%), and stomach cancer (77.8%) (Table 3). We confirmed the head and neck cancer by staining with Biomax head and neck cancer microarrays containing over 150 patient samples (85% positive). The trop2 staining on FFPE colon cancer microarray was low, which might be due to the antigen retrieval method. To verify the colon cancer prevalence, we stained with frozen colon cancer arrays and showed 94% positive by TROP2 antibody staining. These data suggested the positive TROP2 epitope, a potential TROP2 biomarker by IHC staining and clinical indication of cancer types for the clinical trials and treatment.

TABLE 3
IHC Summary of KD005/All Tumors TMAs

| | | No. of positive | IHC Score | | |
|---|---|---|---|---|---|
| Arrays | Organ | Tumors/Total (%) | + | ++ | +++ |
| TMA #1* | Ovary | 5/7 (71.4) | 2 | 1 | 2 |
| NBPS-45052 & | Bladder | 8/8 (100) | 0 | 3 | 5 |
| 45056 | Breast | 8/8 (100) | 4 | 0 | 4 |
| | Kidney | 1/13 (7.7) | 0 | 0 | 1 |
| | Uterus | 5/6 (83.3) | 1 | 3 | 1 |
| | Lung | 7/9 (100) | 2 | 3 | 4 |
| | Colon** | 3/8 (37.5) | 1 | 0 | 2 |
| | Esophagus | 7/9 (77.8) | 0 | 1 | 6 |
| | Liver | 2/9 (22.2) | 1 | 0 | 1 |
| | Rectum | 2/9 (22.2) | 1 | 1 | 0 |
| | Stomach | 7/9 (77.8) | 2 | 4 | 1 |
| | Total Positive | 57/89 (64) | 14 | 16 | 27 |
| TMA #2* | Head and Neck | 101/119 (85) | 40 | 53 | 8 |
| (H&N) | | | | | |
| Biomax HN602 | | | | | |
| & HN601d | | | | | |
| TMA | Colon Cancer | 33/35 (94) | 7 | 15 | 11 |
| Frozen Array | | | | | |
| Biochain | | | | | |
| T6235090 | | | | | |

### Example 12—Crystallization and Structure Determination

### Crystallization

Crystals were obtained by the vapor diffusion method at 18° C., by mixing equal volumes of protein plus well solution.

The TROP2 ECD:VHH 3572 crystals grew in 55.5 mM MES, 45.5 mM Imidazole, 10 mM D-Galactose, 10 mM D-Glucose, 10 mM D-Mannose, 10 mM D-Xylose, 10 mM L-Fucose, 10 mM N-acetyl-D-glucosamine, 7% (w/v) PEG 20,000 and 14% (w/v) PEG 500 MME. For cryoprotection, crystals were generally transferred to a solution of mother liquor with the precipitant increased to 10% (w/v) PEG 20,000 and 20% (w/v) PEG 500 MME.

Data sets were collected at the European Synchrotron Radiation Facility (beamline MASSIF-1). Data was processed using XDS (Kabsch, W. (2010a/b) Acta Cryst D66, 125-132) and AIMLESS (Evans, P. R. and Murshudov, G. N. (2013) Acta Cryst D69, 1204-1214) from the CCP4 Suite (Winn, M., et al. (2011) Acta Cryst D67, 235-242).

The crystal structure was solved by molecular replacement using Phaser (McCoy et al., (2005) Acta Cryst D 61, 458-64), using atomic models generated using Alphafold2-multimer (Evans et al., (2022) bioRxiv doi:10.1101/2021.10.04.463034). Atomic models were built using Coot (Emsley, P. & Cowtan, K. Coot, (2004) Acta Cryst D60, 2126-32) and refined with Refmac (Murshudov, et al., (1997) Acta Cryst D53, 240-255) Co-crystals of TROP2 ECD and VHH M100Y were refined to 1.75 Å resolution after merging three datasets (Table 4).

TABLE 4

| | TROP2 + 3572 | |
|---|---|---|
| Data Collection | | |
| Beamline | ESRF MASSIF-1 | |
| Wavelength | 0.965459 | |
| Space Group | P 3₂21 | |
| Cell Dimensions | 81.5, 81.5, 113.6, | |
| a, b, c (Å), α, β, γ (o) | 90, 90, 120 | |
| Resolution (Å) | 70.56-1.75 | |
| Rmerge | 0.150 | (3.923) |
| CC 1/2 | 0.994 | (0.293) |
| I/σI | 7.2 | (0.8) |
| Completeness (%) | 100.0 | (99.9) |
| Redundancy | 9.4 | (6.6) |
| Refinement | | |
| No. of Reflections | 412573 | |
| No. of Unique | 43744 | |
| Rfactor/Rfree (%) | 19.9 | (25.8) |
| Wilson B-factors (Å) | 39.11 | |
| B-factors (Å) | | |
| Protein | 52.23 | |
| R.M.S. Deviations | | |

| | |
|---|---|
| Bond lengths (Å) | 0.0079 |
| Bond angles (°) | 1.458 |

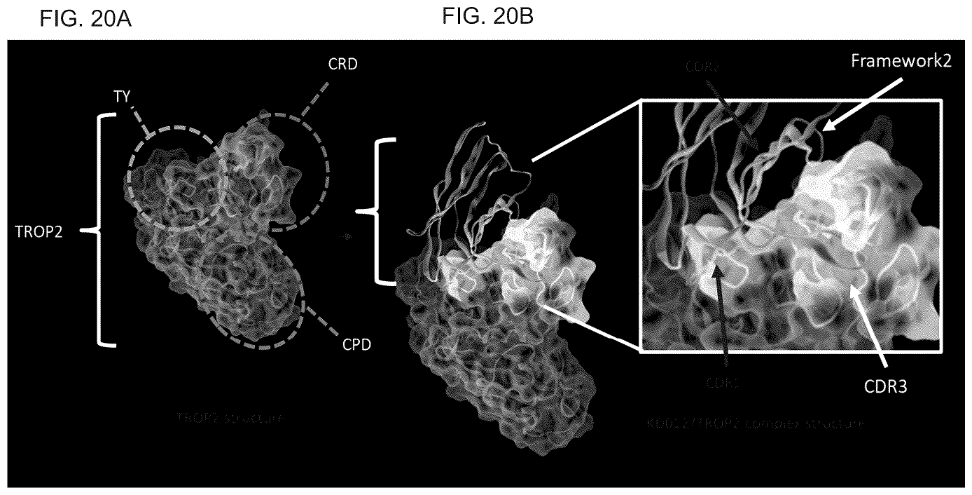

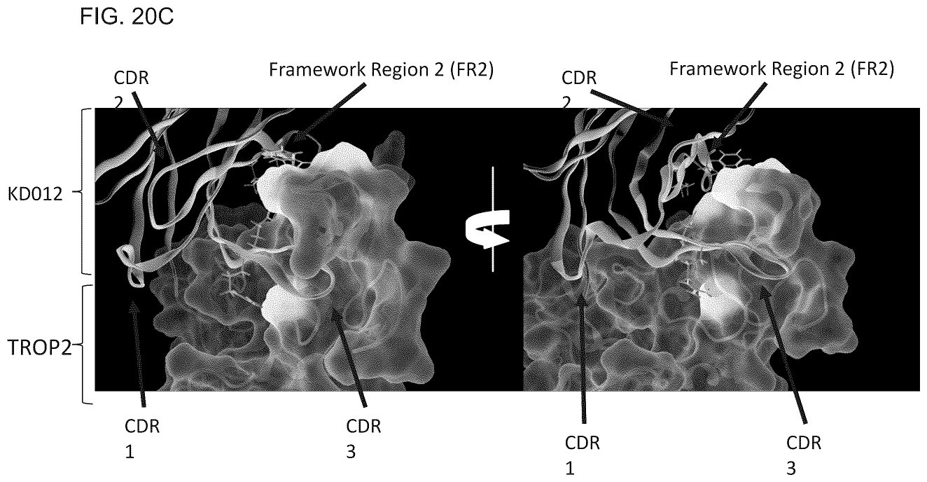

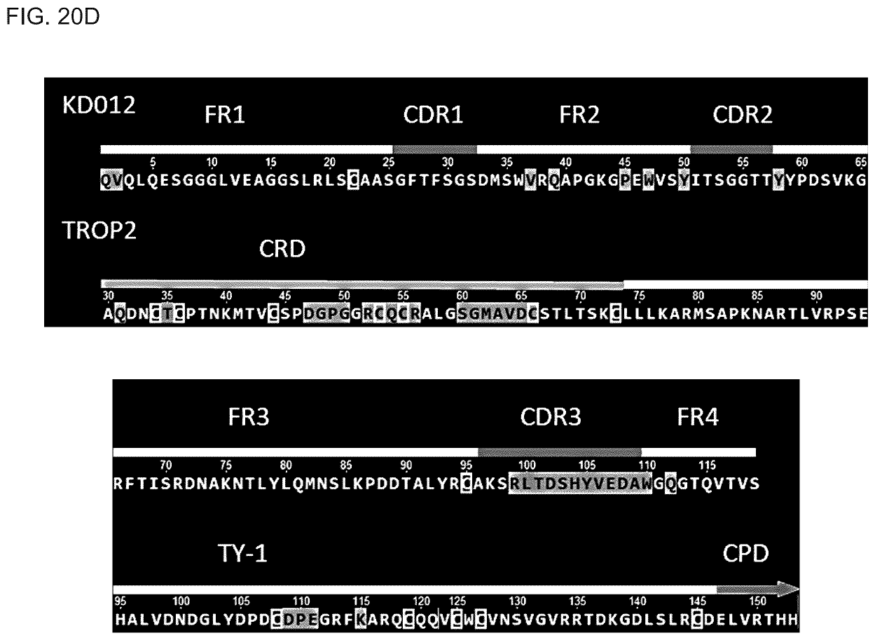

As can be seen from FIGS. 20A-20C, the CDR3 and FR2 region of the variable region of KD005 (i.e., KD012) appears to surround a knob formed by the cysteine-rich domain of TROP2. The CDR3 of KD012 forms a clip fitting into a groove within the CRD of the TROP2 protein which may explain the high-affinity binding and slow dissociation rate. Furthermore, the framework region 2 of the KD012 sdAb also contributes to the binding to TROP2 which appears to be an unusual feature.

Since KD001, KD002, KD003, KD004 and KD005 compete with each other, it is expected that they all form a similar structure and have similar binding affinity. It is also unique that the framework 2 of KD012 contribute to the binding to TROP2 protein.

These structural data supported the high affinity of these anti-TROP2 sdAbs and potent in vivo efficacy.

The co-crystal structured revealed amino acid residues of KD012 and TROP2 that are involved in this interaction (FIG. 20D).

Table 5A shows the amino acid residues of the variable region of KD005 (i.e., corresponding to KD012: SEQ ID NO: 6) that interact with human TROP2.

TABLE 5A

| | Position | Location | Residues | Position | Location | Residues | | |
|---|---|---|---|---|---|---|---|---|
| Type | KD012 | KD012 | KD012 | TROP2 | TROP2 | TROP2 | Energy | Distance |
| D | 1 | FR1 | Gln1 | 80 | TY-1 | Asp109 | −0.22 | 3.81 |
| D | 1 | FR1 | Gln1 | 81 | TY-1 | Pro110 | −0.1 | 3.9 |
| D | 1 | FR1 | Gln1 | 82 | TY-1 | Glu111 | −0.27 | 3.86 |
| D | 2 | FR1 | Val2 | 81 | TY-1 | Pro110 | −0.31 | 4.01 |
| DH | 2 | FR1 | Val2 | 82 | TY-1 | Glu111 | −6.95 | 3.82 |
| D | 37 | FR2 | Val37 | 27 | CRD | Arg56 | −0.98 | 4.13 |
| D | 39 | FR2 | Gln39 | 31 | CRD | Ser60 | 0.01 | 4.26 |
| D | 45 | FR2 | Pro45 | 27 | CRD | Arg56 | 1.14 | 4.09 |
| D | 45 | FR2 | Pro45 | 31 | CRD | Ser60 | −0.01 | 4.2 |
| D | 45 | FR2 | Pro45 | 32 | CRD | Gly61 | 0.94 | 3.95 |
| DH | 47 | FR2 | Trp47 | 18 | CRD | Asp47 | −5.09 | 3.68 |
| D | 47 | FR2 | Trp47 | 19 | CRD | Gly48 | 0.66 | 3.92 |
| DA | 47 | FR2 | Trp47 | 20 | CRD | Pro49 | −4.89 | 3.97 |
| D | 47 | FR2 | Trp47 | 25 | CRD | Gln54 | 1.92 | 3.87 |
| D | 47 | FR2 | Trp47 | 27 | CRD | Arg56 | 0.7 | 4.09 |
| D | 50 | FR2 | Tyr50 | 20 | CRD | Pro49 | 2.66 | 3.93 |
| D | 58 | FR3 | Tyr58 | 20 | CRD | Pro49 | 0.71 | 4.23 |
| D | 58 | FR3 | Tyr58 | 21 | CRD | Gly50 | 1.96 | 3.91 |
| DIH | 99 | CDR3 | Arg99 | 36 | CRD | Asp65 | −19.2 | 3.47 |
| D | 100 | CDR3 | Leu100 | 20 | CRD | Pro49 | −0.54 | 4.35 |
| D | 100 | CDR3 | Leu100 | 23 | CRD | Arg52 | −1.27 | 4.03 |
| DH | 101 | CDR3 | Thr101 | 23 | CRD | Arg52 | −9.92 | 3.69 |
| D | 101 | CDR3 | Thr101 | 36 | CRD | Asp65 | −0.68 | 4.06 |
| D | 102 | CDR3 | Asp102 | 23 | CRD | Arg52 | −0.04 | 3.92 |
| D | 103 | CDR3 | Ser103 | 2 | CRD | Gln31 | −0.02 | 4.13 |
| D | 103 | CDR3 | Ser103 | 23 | CRD | Arg52 | −0.43 | 4.03 |
| D | 103 | CDR3 | Ser103 | 24 | CRD | Cys53 | −0.12 | 3.85 |
| DH | 104 | CDR3 | His104 | 6 | CRD | Thr35 | −4.23 | 3.51 |
| D | 104 | CDR3 | His104 | 23 | CRD | Arg52 | 0.28 | 4.02 |
| DH | 104 | CDR3 | His104 | 24 | CRD | Cys53 | −1.17 | 3.91 |
| DH | 104 | CDR3 | His104 | 26 | CRD | Cys55 | −0.97 | 3.93 |
| DA | 104 | CDR3 | His104 | 37 | CRD | Cys66 | 0.07 | 3.88 |
| D | 105 | CDR3 | Tyr105 | 19 | CRD | Gly48 | 0.32 | 4.49 |
| D | 105 | CDR3 | Tyr105 | 20 | CRD | Pro49 | 2.68 | 4.06 |
| D | 105 | CDR3 | Tyr105 | 23 | CRD | Arg52 | 1.8 | 4.11 |
| DH | 105 | CDR3 | Tyr105 | 24 | CRD | Cys53 | −5.53 | 3.87 |
| D | 105 | CDR3 | Tyr105 | 25 | CRD | Gln54 | 0.77 | 4.13 |
| D | 105 | CDR3 | Tyr105 | 27 | CRD | Arg56 | 1.5 | 3.69 |
| D | 105 | CDR3 | Tyr105 | 34 | CRD | Ala63 | 0.75 | 4.1 |
| D | 105 | CDR3 | Tyr105 | 35 | CRD | Val64 | 0.03 | 4.13 |
| D | 106 | CDR3 | Val106 | 34 | CRD | Ala63 | −0.3 | 3.97 |
| D | 106 | CDR3 | Val106 | 35 | CRD | Val64 | −0.27 | 4.09 |
| D | 106 | CDR3 | Val106 | 36 | CRD | Asp65 | −0.64 | 4.16 |
| DIH | 107 | CDR3 | Glu107 | 27 | CRD | Arg56 | −37.12 | 3.27 |
| D | 107 | CDR3 | Glu107 | 32 | CRD | Gly61 | 0.02 | 4.16 |
| D | 107 | CDR3 | Glu107 | 33 | CRD | Met62 | −1.04 | 3.89 |
| DH | 107 | CDR3 | Glu107 | 34 | CRD | Ala63 | −9.04 | 3.9 |
| D | 108 | CDR3 | Asp108 | 33 | CRD | Met62 | −0.27 | 4.43 |
| D | 108 | CDR3 | Asp108 | 81 | TY-1 | Pro110 | 0.01 | 4.29 |
| D | 108 | CDR3 | Asp108 | 82 | TY-1 | Glu111 | −0.47 | 3.81 |
| D | 109 | CDR3 | Ala109 | 33 | CRD | Met62 | −0.79 | 4.25 |
| D | 109 | CDR3 | Ala109 | 81 | TY-1 | Pro110 | −0.68 | 3.78 |
| D | 109 | CDR3 | Ala109 | 82 | TY-1 | Glu111 | 0.01 | 4.2 |
| D | 110 | FR4 | Trp110 | 27 | CRD | Arg56 | −0.26 | 4.37 |
| D | 110 | FR4 | Trp110 | 31 | CRD | Ser60 | −0.65 | 3.79 |
| D | 110 | FR4 | Trp110 | 32 | CRD | Gly61 | −0.48 | 4.11 |
| D | 110 | FR4 | Trp110 | 33 | CRD | Met62 | −5.73 | 4.1 |
| DH | 110 | FR4 | Trp110 | 81 | TY-1 | Pro110 | −3.73 | 3.94 |
| D | 112 | FR4 | Gln112 | 86 | TY-1 | Lys115 | −0.09 | 4.22 |

TABLE 5B

| | | |
|---|---|---|
| D | distance | VdW distance interaction energies. |
| A | arene | Arene interactions in the list, these include π:π, π-H, |
| | | and π:cation contacts |
| I | ionic | Ionic bond contacts in the list |
| H | hbond | Hydrogen bond contacts |

### KD102 Annotated According to IMGT Numbering

This data is one representative example of protein-protein interactions determined from the co-crystal structure done in triplicate. Slight variation in the distance and energy values are likely due to the energy minimization step performed in QuickPrep in MOE.

### Example 13—Antibody Humanization and Characterization

### ELISA Binding Assay

Human TROP2 recombinant proteins were coated on 96-well ELISA plates at the concentration of 1 μg/mL. Plates were covered with adhesive plate cover and stored at 4° C. overnight. The next day, plates were washed 3x with washing buffer (PBS with 0.1% Tween™ 20). Plates were blocked with 2% fat-free BSA in washing buffer for 1 hour at 37° C. During the incubation time, the TROP2 reference antibodies, as well as the humanized variants, were diluted in the blocking buffer to the indicated concentrations. In brief, the anti-TROP2 sdAbs starting concentration was 200 nM and serial diluted 1 in 5 to 0.00256 nM. After blocking, the blocking buffer was removed from the wells and 100 μL of anti-TROP2 sdAbs dilutions were transferred to the corresponding wells. Plates were covered with adhesive plates cover and incubated at 37° C. for 1 hour. During the primary antibody incubation, the secondary antibody (goat anti-human IgG, HRP conjugated) was diluted 1 in 5000 with the blocking buffer. Plates were washed 3x with blocking buffer, followed by secondary antibody incubation for another 1 hour at 37° C. After the secondary antibody incubation, plates were washed 3x with washing buffer and added with 100 μL of ELISA chemiluminescent substrate (Thermofisher). The luminescence signal was detected by SpectraMax i3x at 425 nM.

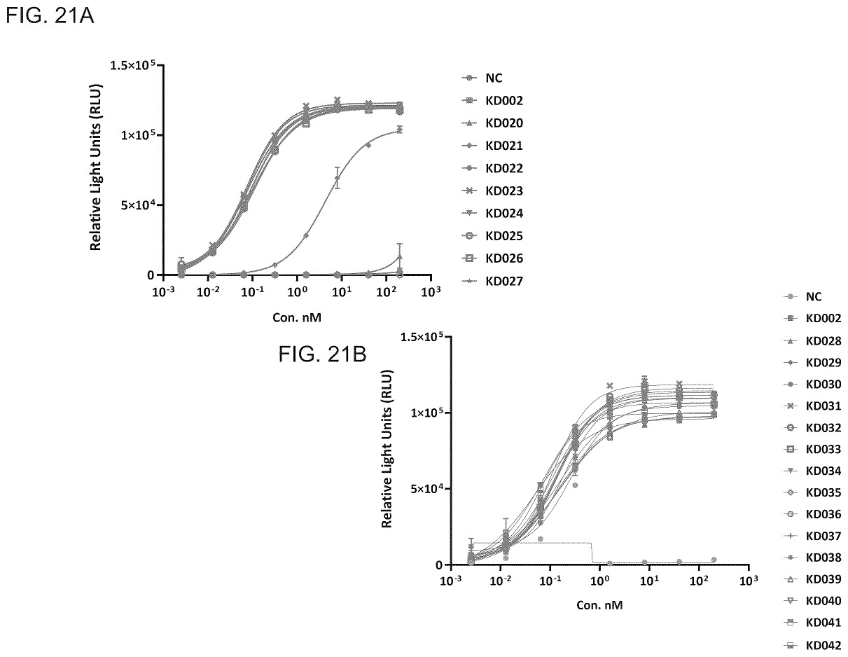

As illustrated in FIG. 21, all humanized variants of KD002 showed similar binding to KD002 except for KD021 and KD020, which showed reduced binding to recombinant human TROP2 protein in this assay. EC50 values are provided in Table 6.

TABLE 6

| KD002 variants | EC50 (nM) |
|---|---|
| KD002 | 0.07 |
| KD020 | N/A |
| KD021 | 4.30 |
| KD022 | 0.07 |
| KD023 | 0.08 |
| KD024 | 0.09 |
| KD025 | 0.11 |
| KD026 | 0.10 |
| KD027 | 0.09 |
| KD028 | 0.14 |
| KD029 | 0.16 |
| KD030 | 0.12 |
| KD031 | 0.11 |
| KD032 | 0.15 |
| KD033 | 0.13 |
| KD034 | 0.13 |
| KD035 | 0.12 |
| KD036 | 0.13 |
| KD037 | 0.18 |
| KD038 | 0.26 |
| KD039 | 0.15 |
| KD040 | 0.05 |
| KD041 | 0.15 |
| KD042 | 0.07 |

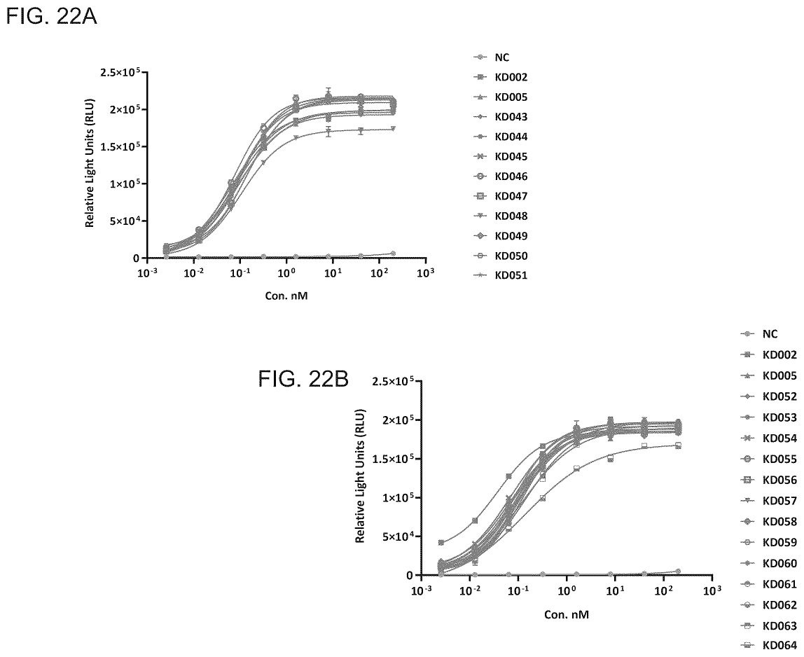

As illustrated in FIGS. 22A and 22B, all humanized variants showed similar binding to KD005 except for KD064, which showed reduced binding EC50 and maximum binding signal in this assay. Since the amino acid changes in KD064 are at the framework 2, this implies the importance of framework binding to TROP2 in addition to the CDR3. EC50 values and % EC50 value compared to KD005 are presented in Table 7.

TABLE 7

| Code name | EC50 (nM) | EC50% to KD005 | RMax |
|---|---|---|---|
| KD002 | 0.055 | 0.6875 | 199906.5 |
| KD005 | 0.08 | 1 | 202140.5 |
| KD043 | 0.09 | 1.125 | 195093 |
| KD044 | 0.08 | 1 | 200020 |
| KD045 | 0.09 | 1.125 | 206168.5 |
| KD046 | 0.08 | 1 | 206369.5 |
| KD047 | 0.14 | 1.75 | 207446.5 |
| KD048 | 0.11 | 1.375 | 173921.5 |
| KD049 | 0.14 | 1.75 | 212138 |
| KD050 | 0.1 | 1.25 | 211592 |
| KD051 | 0.12 | 1.5 | 211126 |
| KD052 | 0.07 | 0.875 | 189730 |
| KD053 | 0.1 | 1.25 | 198757 |
| KD054 | 0.07 | 0.875 | 195032.5 |
| KD055 | 0.08 | 1 | 194513 |
| KD056 | 0.09 | 1.125 | 190663.5 |
| KD057 | 0.09 | 1.125 | 183824.5 |
| KD058 | 0.1 | 1.25 | 183885.5 |
| KD059 | 0.1 | 1.25 | 188315.5 |
| KD060 | 0.11 | 1.375 | 190929 |
| KD061 | 0.12 | 1.5 | 191304 |
| KD062 | 0.14 | 1.75 | 189381 |
| KD063 | 0.12 | 1.5 | 184553 |
| KD064 | 0.14 | 1.75 | 167056.5 |

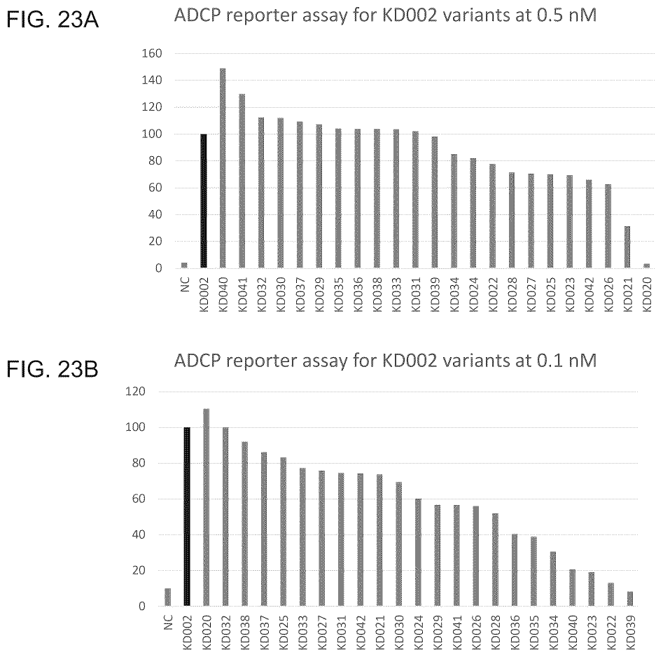

ADCP assays were carried out as described above. Results of assays for ADCP activity for KD002 and humanized variants are presented in FIGS. 23A and 23B. Antibody concentration of 0.1 nM and 0.5 nM were selected as 0.1 nM is close to the EC50 and 0.5 nM is close to the plateau observed. As illustrated in FIGS. 23A and 23B, the humanized variants KD020 and KD021 had a significantly reduced ADCP activity, consistent with the binding data. Some of the amino acid substitution in KD020 are located in the framework 2 and may help explaining the decreased binding as framework 2 contributes to TROP2 binding as revealed by the structure data.

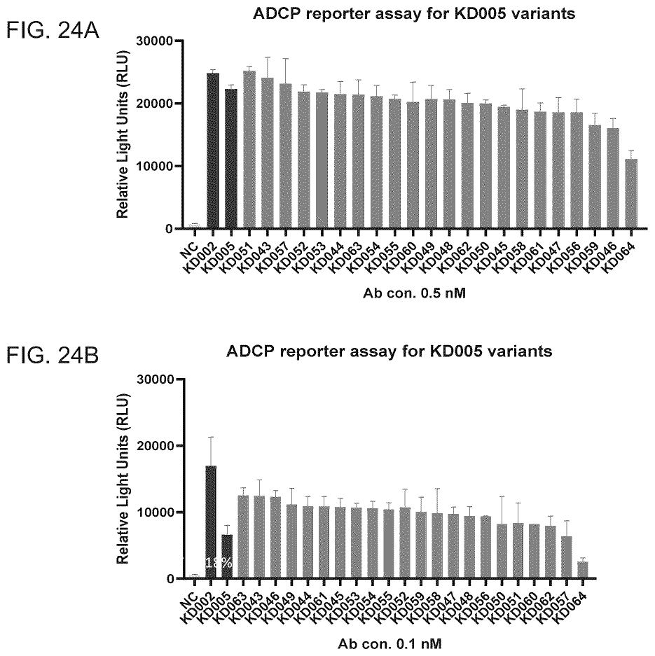

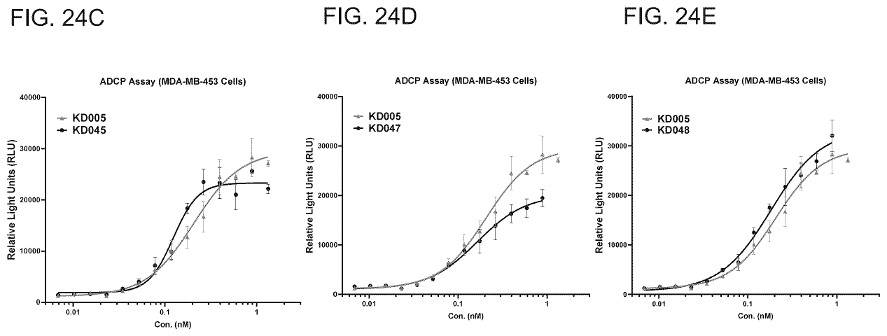

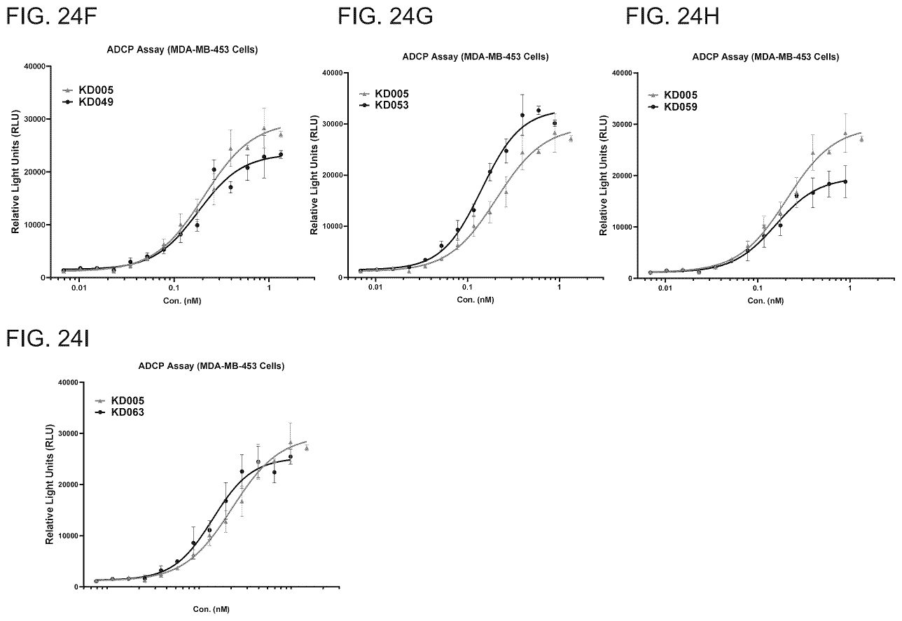

Results of assays for ADCP activity for KD005 and humanized variants are presented in FIGS. 24A and 24B. The antibody concentration of 0.1 nM and 0.5 nM were selected as 0.1 nM is close to the EC50 and 0.5 nM is close to the plateau. At concentration of 0.1 nM, the majority of KD005 humanized variants exhibited better ADCP activity than the reference antibody KD005 except for the KD064. At concentration of 0.5 nM, the activity appeared essentially equivalent except for the KD064. These results are consistent with the binding data as KD064 significantly reduced the binding to TROP2 after framework2 mutation.

However, dose-response ADCP analysis FIGS. 24C-24I carried out on selected KD005 humanized variants (KD045, KD047, KD048, KD049, KD053, Kd059 and KD063) revealed that some had slightly ADCP better activity, while others had similar ADCP activity or lower ADCP activity.

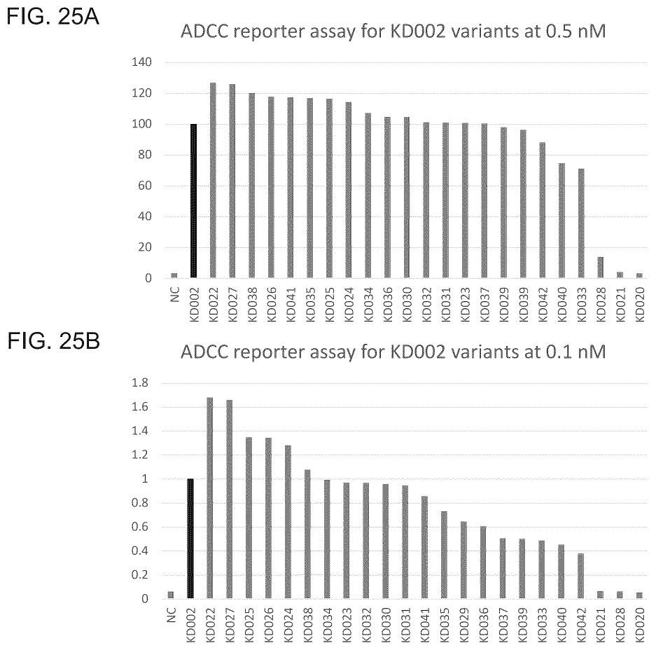

The ADCC activity of the humanized variants was evaluated as described above. Results of ADCC activity for KD002 and KD002 humanized variants are presented in FIGS. 25A and 25B. Antibody concentration of 0.1 nM and 0.5 nM were selected as 0.1 nM is close to the EC50 and 0.5 nM is close to the plateau. At 0.5 nM concentration, the majority of humanized variants had similar ADCC activity to KD002 except for KD020 and KD021. The explanation is that the ADCC activity showed saturation at 0.5 nM close to the plateau. ADCC activity at 0.1 nM showed greater variability than at 0.5 nM. The humanized variants KD022, KD027, KD025, KD026, and KD024 exhibited better ADCC activity than KD002. Whereas KD028, KD020 and KD021 have a significantly reduced ADCC activity, which was consistent with the binding data.

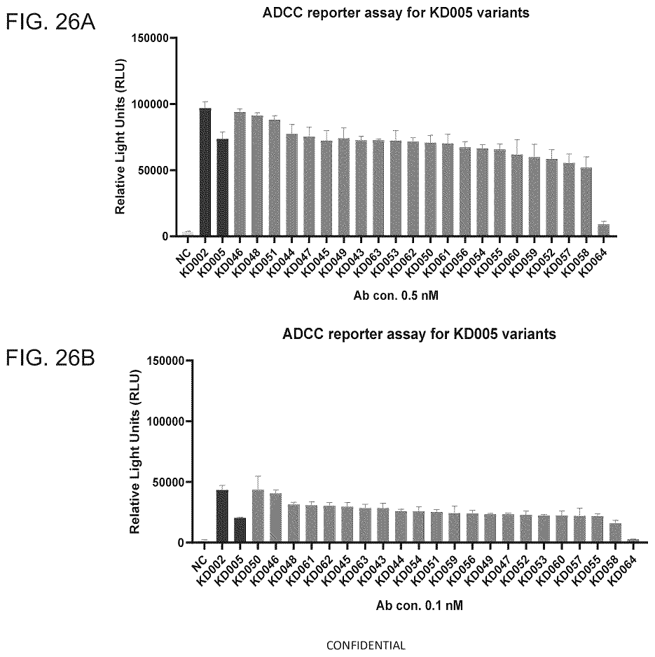

Results of ADCC activity for KD005 and KD005 humanized variants are presented in FIGS. 26A and 26B. Antibody concentration of 0.1 nM and 0.5 nM were selected as 0.1 nM is close to the EC50 and 0.5 nM is close to the plateau. At the concentration of 0.1 nM, the majority of KD005 humanized variants exhibited similar or better ADCC activity than the reference antibody KD005 except for the KD064. This is consistent with the binding data as KD064 significantly reduced the binding to TROP2 after framework2 mutation.

Based on the above results KD002 and KD005 humanized variants were ranked according to binding data, ADCP activity and/or ADCC activity.

For example, based on ELISA binding data, it appears that KD056, KD057, KD050, KD055, KD043, KD045, KD052, KD054, KD044, KD046 have a EC50 that is similar to or better than KD005, whereas KD054, KD043, KD053, KD044, KD045, KD046, KD047, KD051, KD050 and KD049 have a Rmax that is similar to or better than KD005.

Therefore, based on binding data, it appears that KD043, KD044, KD045, KD046, KD047, KD049, KD050, KD051, KD052, KD053, KD054, KD055, KD056, and KD057 represent the best binders.

The binding EC50 and the maximum binding signal (Rmax) of selected KD005 humanized variants based on the ELISA binding curve is presented in Table 8. The binding EC50 and Rmax were normalized to the reference antibody KD005.

TABLE 8

| Variant | | Variant | |
|---|---|---|---|
| Designation | EC50 (% KD005) | Designation | Rmax (% KD005) |
| KD056 | 1.21 | KD054 | 0.964 |
| KD057 | 1.15 | KD043 | 0.965 |
| KD050 | 1.12 | KD053 | 0.983 |
| KD055 | 1.12 | KD054 | 0.990 |
| KD043 | 1.01 | KD005 | 1 |
| KD045 | 1.01 | KD045 | 1.0199 |
| KD005 | 1 | KD046 | 1.0209 |
| KD052 | 0.93 | KD047 | 1.0262 |
| KD054 | 0.93 | KD051 | 1.0445 |
| KD044 | 0.9 | KD050 | 1.0468 |
| KD046 | 0.9 | KD049 | 1.0495 |

Based on ADCP assays performed herein, it appears that KD043, KD044, KD045, KD046, KD048, KD049, KD051, KD052, KD053, KD054, KD055, KD057, KD61, and KD063 have the best ADCP activity.

The ADCP activity of KD005 humanized variants at 0.1 nM and 0.5 nM are presented in Table 9 and Table 10. The data was normalized to the reference antibody KD005.

TABLE 9
ADCP activity measured at 0.1 nM and 0.5 nM relative
to KD005 ranked based on highest value at 0.1 nM.

| | 0.5 nM | 0.1 nM |
|---|---|---|
| | (% KD005) | (% KD005) |
| KD063 | 96.1 | 189.9 |
| KD043 | 108.1 | 189.0 |
| KD046 | 72.1 | 186.4 |
| KD049 | 92.8 | 169.1 |
| KD044 | 96.4 | 165.0 |
| KD061 | 83.8 | 164.7 |
| KD045 | 87.3 | 163.5 |
| KD052 | 98.2 | 162.6 |
| KD053 | 97.6 | 161.7 |
| KD054 | 94.9 | 160.5 |
| KD005 | 100.0 | 100.0 |

TABLE 10
ADCP activity measured at 0.1 nM and 0.5 nM relative
to KD005 ranked based on highest value at 0.5 nM.

| | 0.5 nM | 0.1 nM |
|---|---|---|
| | (% KD005) | (% KD005) |
| KD051 | 113.1 | 126.9 |
| KD043 | 108.1 | 189.0 |
| KD057 | 103.9 | 96.6 |
| KD005 | 100.0 | 100.0 |
| KD052 | 98.2 | 162.6 |
| KD053 | 97.6 | 161.7 |
| KD044 | 96.4 | 165.0 |
| KD063 | 96.1 | 189.9 |
| KD054 | 94.9 | 160.5 |
| KD055 | 93.0 | 157.9 |
| KD049 | 92.8 | 169.1 |

Based on ADCC assay performed at 0.1 nM and 0.5 nM (EC50 and % Emax relative to KD005), it appears that KD043, KD044, KD045, KD046, KD047, KD048, KD049, KD050, KD051, KD053, KD054, KD061, KD062, and KD063 have the best ADCC activity.

Tables 11 and Table 12 summarize the ADCC activity of KD005 humanized variants at 0.1 nM and 0.5 nM. The data was normalized to the reference antibody KD5.5.

TABLE 11
ADCC activity measured at 0.1 nM and 0.5 nM relative
to KD005 ranked based on highest value at 0.1 nM.

| | 0.5 nM | 0.1 nM |
|---|---|---|
| | (% KD005) | (% KD005) |
| KD050 | 96.3 | 214.3 |
| KD046 | 127.8 | 199.9 |
| KD048 | 124.2 | 154.5 |
| KD061 | 95.5 | 151.4 |
| KD062 | 97.5 | 150.1 |
| KD045 | 98.3 | 145.7 |
| KD063 | 98.7 | 140.1 |
| KD043 | 98.8 | 139.8 |
| KD044 | 105.4 | 127.3 |
| KD054 | 90.3 | 126.8 |
| KD005 | 100.0 | 100.0 |

TABLE 12
ADCC activity measured at 0.1 nM and 0.5 nM relative
to KD005 ranked based on highest value at 0.5 nM.

| | 0.5 nM | 0.1 nM |
|---|---|---|
| | (% KD005) | (% KD005) |
| KD046 | 127.8 | 199.9 |
| KD048 | 124.2 | 154.5 |
| KD051 | 120.0 | 124.5 |
| KD044 | 105.4 | 127.3 |
| KD047 | 102.5 | 115.5 |
| KD049 | 100.8 | 115.7 |
| KD005 | 100.0 | 100.0 |
| KD043 | 98.8 | 139.8 |
| KD063 | 98.7 | 140.1 |
| KD053 | 98.3 | 110.8 |
| KD045 | 98.3 | 145.7 |

### Example 14—Sequence Analysis

Co-crystallization of the variable region of KD005 with human TROP2 reveals that amino acid residues of CDRH3 and FR2 are in close contact with the CRD region of TROP2 (see Table 5 and FIGS. 20B-20D). Amino acid residues of TY-1 (74-146) appear to be involved in the interaction but to a lesser extent, whereas it appears that there is no significant interaction with amino acid residues of the CPD (147-274).

The competition assays and FACS analysis indicate that KD001-KD005 all bind to the same or overlapping regions of TROP2. Therefore, KD001, KD002, KD003 and KD004 may likely bind some or all interacting amino acid residues of human TROP2 identified for KD005.

Since KD002 variants have similar properties as KD002, it is expected that KD002 variants also bind to the CRD region of TROP2. As well, since KD005 variants have similar properties as KD002, it is expected that KD005 variants also bind to the CRD region of TROP2.

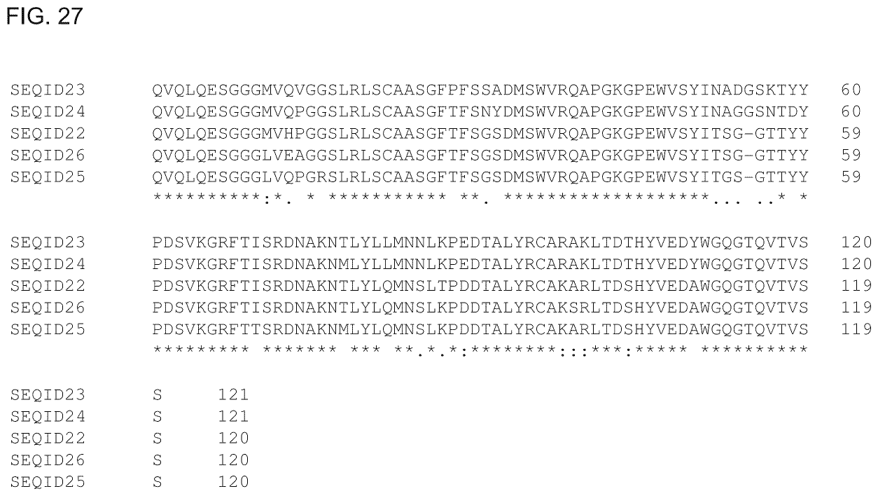

An alignment between the variable regions of KD001-KD005 (FIG. 27: SEQ ID NO:22, SEQ ID NO:23, SEQ ID NO:24, SEQ ID NO:25 or SEQ ID NO:26) was generated. The alignment was used to generate an exemplary consensus sequence (SEQ ID NO:145) carrying 25 amino acid substitutions out of 120 or 121 positions.

KD001-KD005 are therefore approximately 80% identical to each other over the entire length of the polypeptide.

As illustrated in FIG. 27, the variable region of KD001-KD005 have at least 80% sequence identity to each other over the entire length of the polypeptide.

In the alignment of FIG. 27 as well as in other alignments presented herein a colon (:) indicates conservation between groups of strongly similar properties as below—roughly equivalent to scoring >0.5 in the Gonnet PAM 250 matrix (Table 13A). A period (.) indicates conservation between groups of weakly similar properties as below—roughly equivalent to scoring=<0.5 and >0 in the Gonnet PAM 250 matrix (Table 13B). For example, it may be possible to substitute an amino acid residue identified as being part of a group in Table 13A for another of the same group. Similarly, it may be possible to substitute an amino acid residue identified as being part of a group in Table 13B for another of the same group.

An empty space indicates that the amino acid may be a non-conservative amino acid substitution.

TABLE 13A
(:)

STA
NEQK
NHQK
NDEQ
QHRK
MILV
MILF
HY
FYW

TABLE 13B
(.)

CSA
ATV
SAG
STNK
STPA
SGND
SNDEQK
NDEQHK
NEQHRK
FVLIM
HFY

Upon analysis of the sequence, it appears that KD001-KD005 all have an identical IMGT FR2 amino acid sequence, but different IMGT CDRH1, IMGT CDRH2 and IMGT CDRH3 or different combinations of IMGT CDRH1, IMGT CDRH2 and IMGT CDRH3.

For example, KD001, KD004 and KD005 have identical IMGT CDRH1 (SEQ ID Nos: 52, 59 and 62 are identical) whereas KD002 and KD003 have unique IMGT CDRH1 sequences.

KD001 and KD005 have identical IMGT CDRH2 (SEQ ID Nos: 50 and 53 are identical) whereas KD002, KD003 and KD004 have unique IMGT CDRH2 sequences.

KD002 and KD003 have identical IMGT CDRH3 (SEQ ID NO:48 and 58 are identical) and KD001 and KD004 have identical IMGT CDRH3, whereas KD005 has a unique IMGT CDRH3.

There is a high level of variability between KD001-KD005 IMGT CDRH1 indicating that this region may not be involved in binding as also illustrated by the co-crystallization experiments.

There is also substantial variability in IMGT CDRH2 indicating that this region may not be involved in binding as also illustrated by the co-crystallization experiments.

There is, however a high level of similarity between the IMGT CDRH3 of KD001-KD005. An alignment between the different IMGT CDRH3 shows that 9/14 amino acid residues are identical and 4/14 amino acid residues are conservative amino acid substitutions. Nevertheless, these alignments show that some level of variation may be tolerated in CDRH3 as well.

The amino acid sequence of KD002 and KD005 variants was also analyzed.

The IMGT CDRH1 (SEQ ID NO:55) and IMGT CDRH3 (SEQ ID NO:48) of KD002 variants is identical to that of KD002.

The CDRH2 of most variants is identical to that of KD002 (SEQ ID NO:47) except for 2 variants having a sequence illustrated in SEQ ID:123 and SEQ ID NO:124 that each includes one amino acid substitution.

Several KD002 variants have an IMGT FR2 identical to that of KD002 (SEQ ID NO:116) while others have one or two amino acid substitutions. Variations in KD002 IMGT FR2 are illustrated in SEQ ID NO: 117, SEQ ID NO: 118 and SEQ ID NO:119.

The IMGT CDRH1, CDRH2 and CDRH3 of KD005 variants are identical to that of KD005.

Several KD005 variants have an IMGT FR2 identical to that of KD005 while others have one or two have one or two amino acid substitutions. Variations in KD005 IMGT FR2 are illustrated in SEQ ID NO: 117, SEQ ID NO: 118 and SEQ ID NO:120.

An alignment between the IMGT FR2 sequences of all tested variants shows that 14/17 amino acid residues are identical. The binding and/or function of these variants was not affected by these substitutions suggesting that FR2 can sustain some level of variation.

However, the amino acid substitutions introduced in the IMGT FR2 of KD064 lead to a reduced binding and activity. The co-crystallization experiment confirms that amino acid residues 37, 45, 47, and 50 of IMGT FR2 are involved in binding with human TROP2. Based on the results obtained for KD064, amino acid substitution V/Y at position 37 and amino acid substitution P/R at position 45 may affect binding.

Overall, the results presented herein show that CDRH3 and FR2 are important for binding to TROP2 but that some level of variation may be tolerated and that CDRH1 and CDRH2 do not appear to be involved in binding to human TROP2.

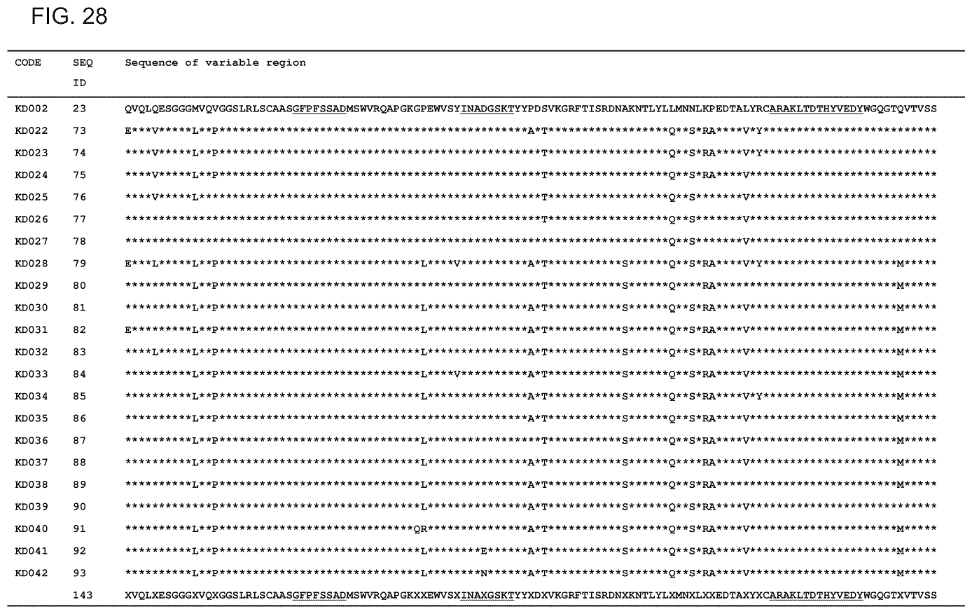

An alignment between the entire variable region of KD002 (SEQ ID NO:23) and the entire variable region of exemplary embodiments of KD002 variants (FIG. 28) was made. The alignment was used to generate an exemplary consensus sequence (SEQ ID NO:143) carrying 18 amino acid substitutions out of the 121 positions.

KD002 and KD002 variants are therefore approximately 85% identical to each other over the entire length of the polypeptide.

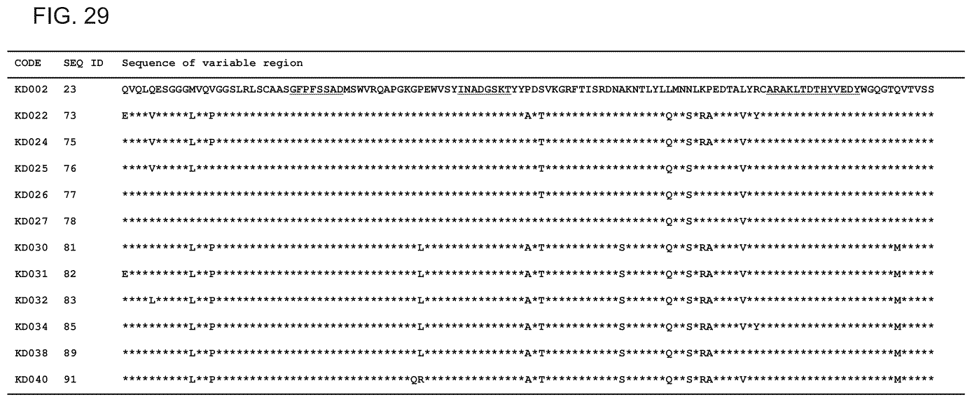

KD002 variants selected for their ADCC and ADCP activity are also illustrated in FIG. 29.

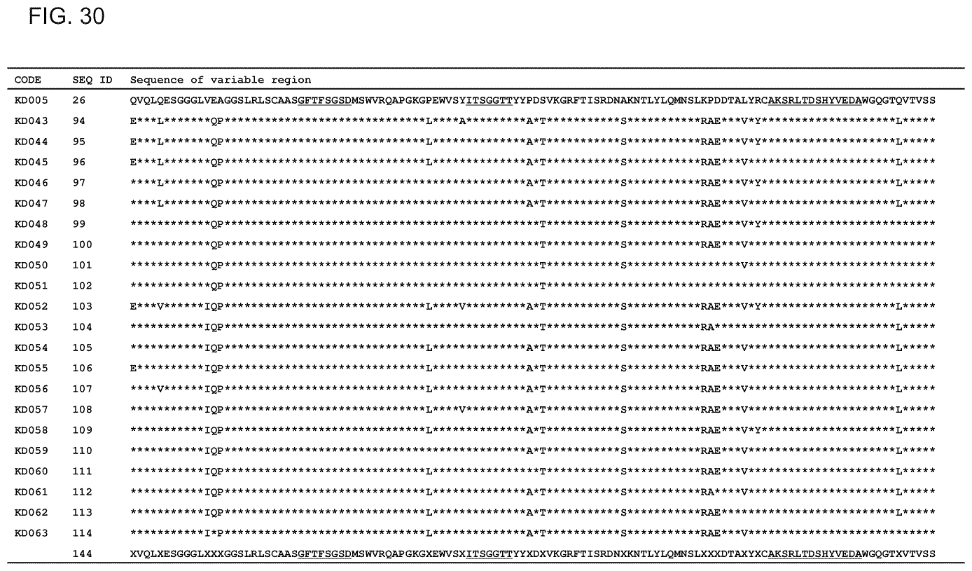

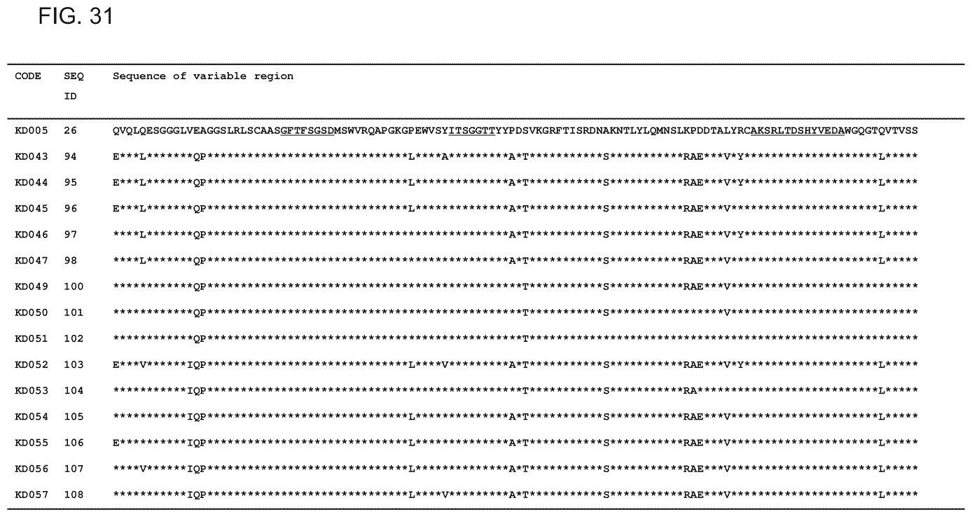

An alignment between the variable region of KD005 (SEQ ID NO:26) and the variable region of exemplary embodiments of KD005 variants (FIG. 30) was also made. The alignment was used to generate an exemplary consensus sequence (SEQ ID NO:144) carrying 16 amino acid substitutions out of the 120 positions.

KD005 and KD005 variants are therefore approximately 85% identical to each other over the entire length of the polypeptide.

KD005 variants selected for their binding activity are illustrated in FIG. 31.

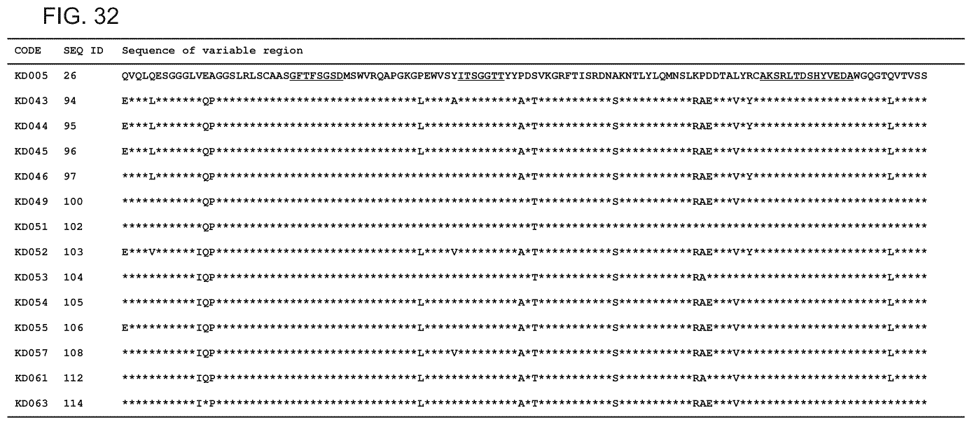

KD005 variants selected for their ADCP activity are illustrated in FIGS. 24C-24I and in FIG. 32.

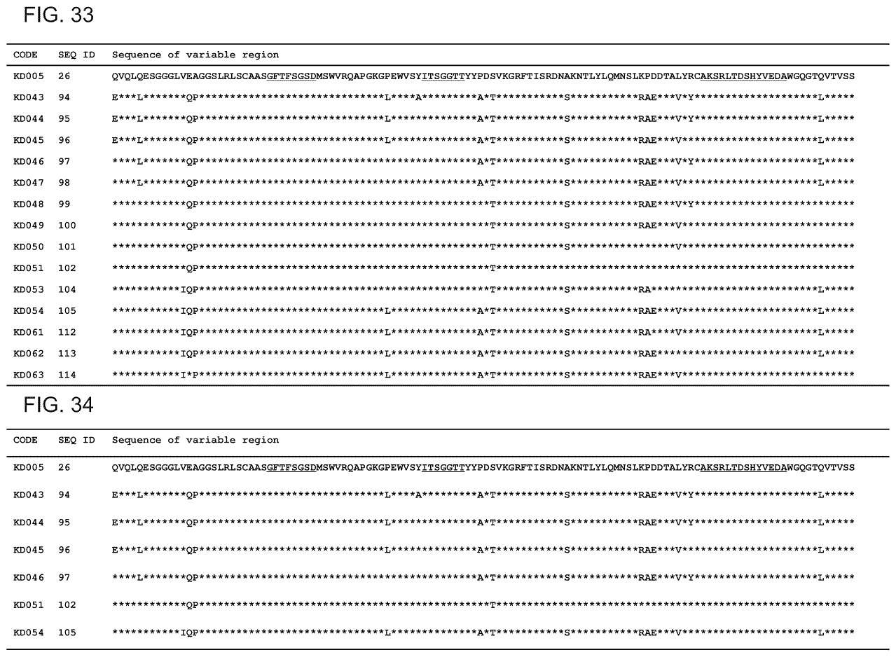

KD005 variants selected for their ADCC activity are illustrated in FIG. 33.

KD005 variants selected for their binding activity, ADCP activity and ADCC activity are illustrated in FIG. 34.

Since they have similar activity and sequences, an alignment between the variable regions of KD001-KD005 (SEQ ID NOs:22-26 and variants (SEQ ID NOs: 94-115) was generated. The alignment was used to generate an exemplary consensus sequence (SEQ ID NO:146) carrying 40 amino acid substitutions out of 121 positions.

All variants are therefore approximately 65% identical to each other over the entire length of the polypeptide. In addition to the embodiments described and provided in this disclosure, the following non-limiting embodiments are particularly contemplated.

### Example 15—Dose Range Finding Intravenous Toxicity Study in Cynomolgus Monkey

The potential toxicity of the TROP2 testing articles was evaluated in the naïve cynomolgus monkeys. All procedures and protocols were carried out under the institutional guidelines of Sinclair Research Center in Missouri, USA. The design is in an accelerated, increasing dosing study. The naïve cynomolgus monkey was first administered intravenously with KD002 or KD005 at the dosing level of 25 mg/kg. If the animal tolerated the first 25 mg/kg dose well, the next dose, 100 mg/kg, was given one week later. If the animal also tolerated 100 mg/kg dose, another two repeating dose of 100 mg/kg were given to the animal once per week.

During the study, animals were examined by the veterinary staff as warranted by clinical signs or other changes. In-cage observations were performed twice daily in the AM and PM (with at least 4 hours between observations) during the in-life study period to assess general health, mortality, or moribundity. Each animal underwent a physical examination, including assessment of behavior, general body condition, condition of major body systems, and skin/coat. Detailed clinical observations were performed at least five times following each dose at 1, 2, 6, 12, and 24 hours post dose and daily on non-dosing days starting after each animal's first dosing event. Serum samples for hematology and clinical chemistry were collected at 2 and 7 days after each dose.

For both KD002 and KD005 testing articles, there were no instances of moribundity or unscheduled deaths during the study. All animals underwent a physical examination and were deemed satisfactory for study inclusion. There were no test article-related effects on body weight relative to dose group. Throughout the dosing phase, all animals had body weights remained with 10% of their weights in the acclimation phase. There were no instances of inappetence either during the study. There were no test article-related findings for any hematology parameters for both KD002 and KD005 studies. All the serum chemistry parameters were in the normal range during the dosing phase to the acclimation phase values for each animal.

Based on the above observations, 100 mg/kg of KD002 and KD005 once weekly for up to at least three doses were well tolerated in cynomolgus non-human primates.

### Example 16—Colorectal Cancer

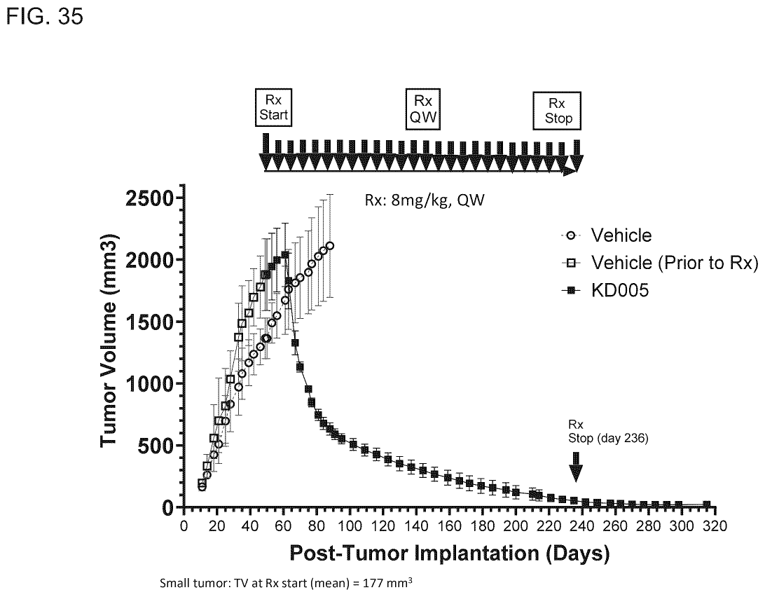

6-8 week-old SCID mice were implanted subcutaneously with Colo201 cells (8 million cells per mouse). When tumors reached approximately 200 mm³ mice were randomized into two groups. Both groups were treated intraperitonially (i.p.) with vehicle only until day 50 post-tumor implantation when one of the groups reached 1800 mm³ and the other reached 1300 mm³. On day 50, the group with average tumor volume of 1800 mm³ started receiving treatment with KD005 at 8 mg/ml once per week and the 1300 mm³ group continued to receive vehicle only. Treatment continued, once per week (QW) until last dose on day 236 post-tumor implantation. As shown in FIG. 35, KD005 treatment caused complete regression of all the tumors.

### Example 17—Non-Small Cell Lung Cancer

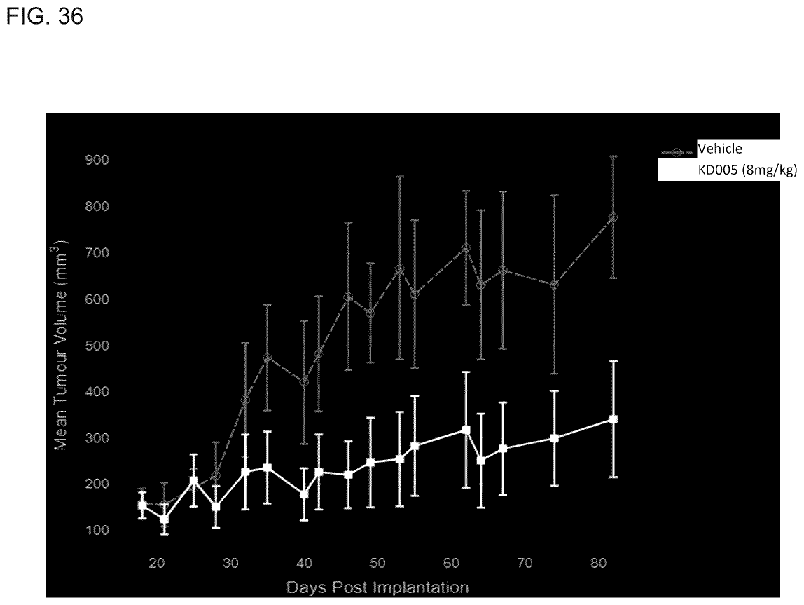

6-8 week-old SCID mice were implanted subcutaneously with NCI-H2228 non-small cell lung cancer cells (5 million cells per mouse). When tumors reached approximately 150 mm³ animals were randomized into two groups. One group started being administered KD005 (8 mg/mL, once per week) and the other with vehicle alone for a total of six doses. As shown in FIG. 36, KD005 treatment caused tumor growth inhibition with a maximum TGI of approximately 80%.

### Example 18—Head and Neck Cancer

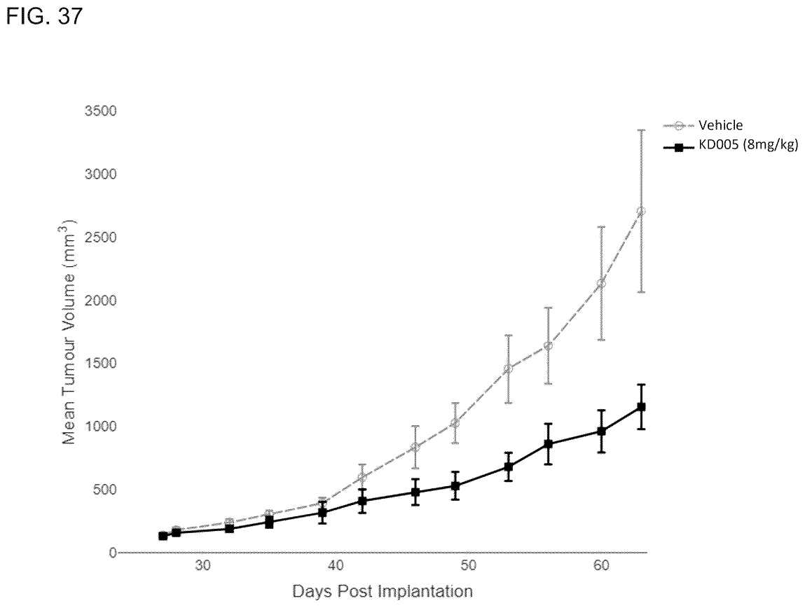

6-8 week-old SCID mice were implanted subcutaneously with Cal27 head and neck cancer cells mixed with Matrigel (1 million cells per mouse). When tumors reached approximately 130 mm³ animals were randomized into two groups. One group started being administered KD005 (8 mg/mL, once per week) and the other with vehicle alone for a total of six doses. As shown in FIG. 37, KD005 treatment caused tumor growth inhibition with a maximum TGI of approximately 50%. Interestingly in all the KD005-treated animals tumors after excision were found not to contain cells and was composed only of a membrane-surrounded liquid-filled sac whereas the tumors in the vehicle-control animals were solid tumor masses.

### Example 19—Colorectal Cancer

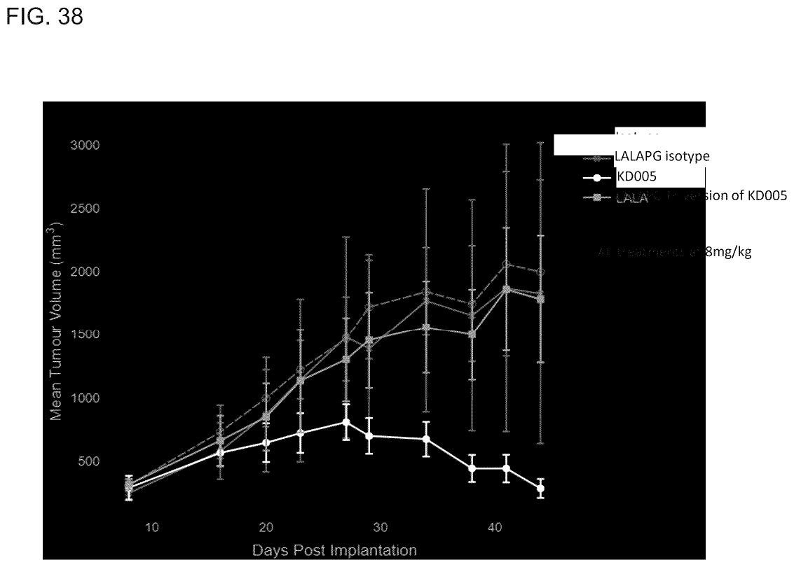

6-8 week-old mice of SCID were implanted subcutaneously with Colo201 colorectal cancer cells (7 million cells per mouse). When tumors reached an average volume of 250 mm³ mice were randomized into four groups. Groups started being administered with KD005, isotype control, a modified version of KD005 with changes in the Fc domain (L243A, L235A and P239G) that renders it effector function-null and the corresponding isotype control with the same amino acid changes (referred to as LALAPG versions), respectively. Antibodies were administered i.v. at 8 mg/kg, once per week for a total of six doses. As shown in FIG. 38, administration of KD005 caused tumor regression of the Colo201 xenografts (with a TGI of 100%) whereas none of the other treatments changed the tumor growth. This result supports the in vivo mechanism of KD005 to be driven by Fc/FcgR-mediated effector function.

### Example 20—NCG and FcgR-Knock Out

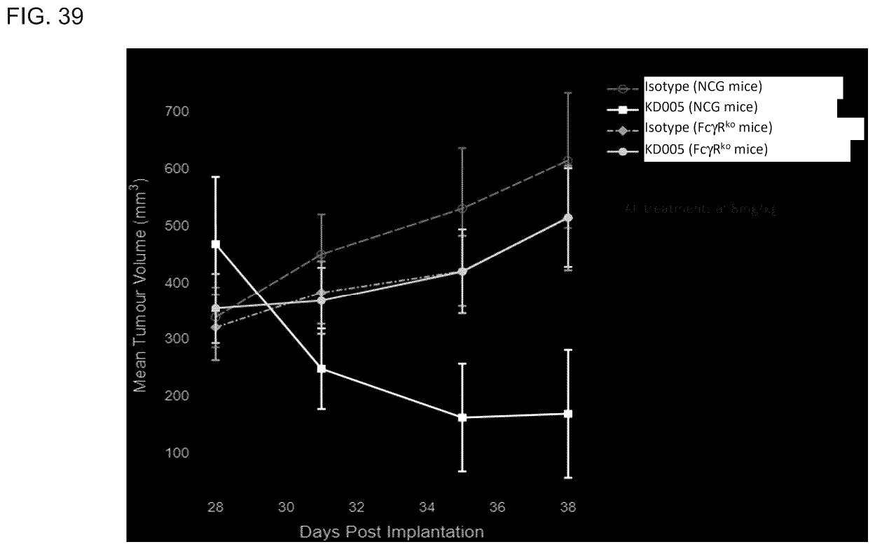

6-8 week-old mice of NCG strain and FcResolv™ mice (from Taconic) of NOG background and deficient in all Fcg Receptors were implanted subcutaneously with MDA-MB-453 breast cancer cells (10 million cells per mouse). When tumors reached a volume between 250 and 500 mm³ mice from each strain were randomized into two groups. One group from each strain were administered KD005 (8 mg/kg, once per week, two doses) and the second group from each mouse strain were administered isotype control. As shown in FIG. 39, KD005 caused tumor regression of the xenografts implanted in the NCG mice (with a TGI of approximately 200% after a single dose) and did not alter tumor growth of the xenografts implanted in the FcResolv™ mice. This supports the in vivo mechanism of action of KD005 to be driven by Fc/FcgR-mediated effector-function.

### Example 21—KD005 Cell Binding and Activity Across a Panel of Cancer Cell Lines

### Cell Binding of KD005

The binding of KD005 across a panel of 38 cancer cell lines was performed (MDA-MB-453, MDA-MB-468, MDA-MB-231, BT-20, HCC38, HCC1187, SK-Br-3, DU4475), Colorectal cancer (LS123, SK-CO-1, DLD-1, COLO 205, COLO 201, HCT-116, SW837, SW480, HT-29, LS1034, LS411N, HCT-15, NCI-H747, SW1417, CL-11, SW620), Head and Neck cancer (BICR-22, FaDu, SCC-9), Ovarian cancer (SK-OV-3, SK-OV-1), Pancreatic cancer (BxPC-3, Capan-1), Gastric cancer (NCI-N87), Esophageal Cancer (T.Tn) and Lung cancer (NCI-H1650, NCI-H292, NCI-H2228, NCI-H226). Cells were harvested, washed twice with 1x PBS and resuspended at 2 million cells/mL and Human TruStain FcX™ (Biolegend, Catalog #422302) was added at 1:100 dilution for 10 minutes to block human Fc receptors. Cells were then washed and seeded at 100,000 cells/well of a 96-well plate. KD005 was added at a final concentration of 8 μg/mL and for 30 minutes on ice. After incubation, cells were washed twice and stained with secondary Alexa Fluor 488 or APC anti-human IgG Fc (Biolegend, Catalog #s 410706 and 410712) for 30 minutes on ice. Cells were then washed twice and resuspended in 100 uL FACS buffer containing 7-Amino-15 Actinomycin D (7-AAD) viability staining solution at 1:100 dilution. Binding to cells was assessed by the fluorescence intensities on cells that were analyzed by BD FACSCanto™ II Flow cytometer (BD Bioscience). Raw Data was collected as mean fluorescent intensity (MFI) for each treatment condition. Relative MFI (MFIR) of KD005 is calculated relative to the isotype control FLA00390. Note that staining with PE anti-human TACSTD2 (TROP2) Antibody (Biolegend, Catalog #363804) was used as a positive control and the TROP2 staining MFIR was calculated relative was calculated relative to the isotype control.

As evidenced by the data of Table 14 the anti-TROP2 sdAb KD005 elicited an ADCP response in 29/38 TROP2-positive cancer cell lines (with ADCP fold induction more than or equal to 3), including MDA-MB-453, MDA-MB-468, MDA-MB-231, BT-20, HCC38, HCC1187, SK-Br-3, LS123, SK-CO-1, DLD-1, COLO 205, COLO 201, HCT-116, SW837, SW480, HT-29, LS1034, LS411N, CL-11, BICR-22, SCC-9, SK-OV-3, SK-OV-1, BxPC-3, NCI-N87, T.Tn, NCI-H1650, NCI-H292 and NCI-H2066.

### ADCP Activity of KD005

The ADCP activity of KD005 was assayed using Promega ADCP kits (cat #CS314906) across a panel of TROP2 expressing cancer cell lines summarized in Table 14; including Triple Negative Breast cancer (MDA-MB-453, MDA-MB-468, MDA-MB-231, BT-20, HCC38, HCC1187, SK-Br-3, DU4475), Colorectal cancer (LS123, SK-CO-1, DLD-1, COLO 205, COLO 201, HCT-116, SW837, SW480, HT-29, LS1034, LS411N, HCT-15, NCI-H747, SW1417, CL-11, SW620), Head and Neck cancer (BICR-22, FaDu, SCC-9), Ovarian cancer (SK-OV-3, SK-OV-1), Pancreatic cancer (BxPC-3, Capan-1), Gastric cancer (NCI-N87), Esophageal Cancer (T.Tn) and Lung cancer (NCI-H1650, NCI-H292, NCI-H2228, NCI-H226). Cells were seeded at 20,000 cells per well of 96-well plates overnight. The next day, media from wells was removed and 25 μL of assay buffer was added. Then, testing antibodies were added at initial concentrations of 5 or 20 nM followed by 9x 1:2.2 serial dilutions prepared in assay buffer. ADCP effector cells (THP-1) were thawed and diluted in assay buffer and 25 μL of cell suspension was added per well of the 96-well plates containing target cell-antibody mix. Plates were incubated for 4 hours at 37 degrees Celsius, 5% CO₂. After incubation, to measure ADCP response, an equal volume (75 μL) of Bio-Glo-NL reagent was added to the wells of 96-well plates and luminescence was recorded using SpectraMax i3x Microplate Reader at an integration time of 500 millisecond per well.

### ADCP Fold Induction=Emax Signal/Emin Signal

### ADCC Activity of KD005

The ADCC activity of KD005 assayed using Promega ADCC kits (cat #G7015) across a panel of TROP2 expressing cancer cell lines summarized in Table 14; including Triple Negative Breast cancer (MDA-MB-453, MDA-MB-468, MDA-MB-231, BT-20, HCC38, HCC1187, SK-Br-3, DU4475), Colorectal cancer (LS123, SK-CO-1, DLD-1, COLO 205, COLO 201, HCT-116, SW837, SW480, HT-29, LS1034, LS411N, HCT-15, NCI-H747, SW1417, CL-11, SW620), Head and Neck cancer (BICR-22, FaDu, SCC-9), Ovarian cancer (SK-OV-3, SK-OV-1), Pancreatic cancer (BxPC-3, Capan-1), Gastric cancer (NCI-N87), Esophageal Cancer (T.Tn) and Lung cancer (NCI-H1650, NCI-H292, NCI-H2228, NCI-H226). In brief, the target cells were collected and using TrypLE and resuspended at the concentration of 0.5 million cell/mL. 12,500 cells were seeded into each well and incubated overnight. The next day, testing antibodies were diluted at initial concentrations of 5 or 20 nM followed by 9x 1:2.2 serial dilutions prepared in assay buffer. The ADCC effector cells were thawed gently in a 37° C. water bath and resuspended into the assay medium. The ratio between effector cells and target cells used was 6:1. Thus 75,000 effector cells were transferred to each well. Plates were incubated at 37° C. incubator for 6 hours. After incubation, plates were equilibrated to room temperature. Bio-Glo luciferase assay substrate was added to each well and incubated at room temperature for 5 minutes. Luminescence signal was measured by SpectraMax i3x plate reader. Data were analyzed by signal fold change upon untreated control wells.

$$ADCC Fold induction = E max Signal / E min Signal$$

TABLE 14

| | | | | KD005 | KD005 | KD005 | KD005 |
|---|---|---|---|---|---|---|---|
| | | KD005 | TROP2 | ADCP EC50 | ADCP | ADCC EC50 | ADCC |
| | Cell Line | MFIR | MFIR | (nM) | Emax | (nM) | Emax |
| Triple | MDA-MB- | 97.09 | 83.22 | 0.146 | 22049 | 0.1525 | 53675 |
| Negative | 453 | | | | | | |
| Breast | MDA-MB- | 79.66 | 1.33 | 0.3166 | 18491 | 0.2485 | 190587 |
| Cancer | 468 | | | | | | |
| | BT-20 | 67.84 | 35.64 | 0.2018 | 7311 | 0.1015 | 203024 |
| | HCC38 | 58.20 | 142.71 | 0.2392 | 23643 | 0.1164 | 401922 |
| | HCC1187 | 24.86 | 4.06 | 0.1433 | 1369 | 0.09797 | 235789 |
| | SK-BR-3 | 21.18 | 28.16 | 0.146 | 242429 | Not tested | Not tested |
| | DU4475 | 2.85 | 0.50 | ~2.494 | 3082 | 0.4083 | 29879 |
| | MDA-MB- | 2.48 | 6.09 | 0.2063 | 3068 | 0.1743 | 422960 |
| | 231 | | | | | | |
| Colorectal | LS123 | 88.69 | 27.06 | 0.1991 | 19929 | 0.07328 | 151398 |
| Cancer | SK-CO-1 | 55.27 | 11.27 | 0.1457 | 17701 | 0.1161 | 67003 |
| | DLD-1 | 54.84 | 29.80 | 0.3407 | 16205 | 0.1578 | 409758 |
| | COLO 205 | 51.09 | 24.39 | 0.2434 | 74625 | Not tested | Not tested |
| | COLO 201 | 38.44 | 18.25 | 0.09464 | 8818 | 0.1835 | 106187 |
| | HCT-116 | 16.87 | 10.41 | 0.101 | 4210 | 0.1092 | 55461 |
| | SW837 | 7.37 | 12.71 | 0.1784 | 63890 | Not tested | Not tested |
| | SW480 | 6.37 | 0.58 | 0.2327 | 6567 | 0.2071 | 200127 |
| | HT-29 | 6.26 | 1.25 | 0.1417 | 5444 | 0.07781 | 153987 |
| | LS1034 | 4.67 | 2.05 | Not tested | Not tested | Not tested | Not tested |
| | LS411N | 4.48 | 11.97 | 0.2157 | 54863 | Not tested | Not tested |
| | HCT-15 | 4.24 | 7.90 | 0.1881 | 15018 | Not tested | Not tested |
| | NCI-H747 | 4.16 | 11.64 | 0.1099 | 17484 | Not tested | Not tested |
| | SW1417 | 3.26 | 9.45 | 0.09413 | 18817 | Not tested | Not tested |
| | CL-11 | 1.05 | 0.51 | Not tested | Not tested | Not tested | Not tested |
| | SW620 | 1.03 | 0.21 | 6.924 | 2144 | 0.1751 | 4150 |
| Head and | BICR-22 | 164.08 | 107.31 | 0.1328 | 23249 | 0.08103 | 320737 |
| Neck | FaDu | 147.98 | 165.59 | 0.1751 | 5381 | 0.06468 | 882147 |
| | SCC-9 | 43.07 | 97.59 | 0.1106 | 14716 | 0.0694 | 635892 |
| Ovarian | SK-OV-3 | 83.11 | 4.57 | 0.197 | 12436 | 0.118 | 416755 |
| Cancer | SK-OV-1 | 55.55 | 38.29 | 0.118 | 6654 | 0.08396 | 90532 |
| Pancreatic | BxPC-3 | 89.98 | 63.33 | 0.2583 | 11685 | 0.1264 | 332801 |
| Cancer | Capan-1 | 32.62 | 21.17 | 0.1952 | 3261 | 0.05707 | 455129 |
| Gastric Cancer | NCI-N87 | 73.00 | 16.44 | 0.1304 | 15270 | 0.05471 | 343086 |
| Esophageal | T.Tn | 172.54 | 130.97 | 0.269 | 11235 | 0.04664 | 445280 |
| Cancer | | | | | | | |
| Lung Cancer | NCI- | 9.38 | 82.23 | 0.1544 | 47887 | Not tested | Not tested |
| | H1650 | | | | | | |
| | NCI-H292 | 64.31 | 79.19 | 0.1628 | 6220 | 0.09007 | 371897 |
| | NCI- | 1.46 | 7.93 | 0.01085 | 15782 | Not tested | Not tested |
| | H2228 | | | | | | |
| | NCI-H226 | 1.04 | 0.38 | 0.2307 | 16695 | Not tested | Not tested |
| | NCI- | 1.30 | 0.53 | Not tested | Not tested | Not tested | Not tested |
| | H2066 | | | | | | |

### Example 22—Binding and Activity of KD001 and Variants

Binding of KD001 Humanized Variants (KD132, KD134) to recombinant human TROP2 protein Human TROP2 recombinant protein was coated on 96-well ELISA plates at the concentration of 1 μg/mL. Plates were covered with adhesive plate cover and stored at 4° C. overnight. The next day, plates were washed 3x with washing buffer (PBS with 0.1% Tween™ 20). Plates were blocked with 2% fat-free BSA in washing buffer for 1 hour at 37° C. During the incubation time, KD001 variants (KD132, KD134) were diluted in the blocking buffer at a starting concentration of 200 nM followed by serial dilution 1 in 5 to 0.00256 nM. After blocking, the blocking buffer was removed from the wells and 100 μL of anti-TROP2 sdAbs dilutions were transferred to the corresponding wells. Plates were covered with adhesive plates cover and incubated at 37° C. for 1 hour. During the primary antibody incubation, the secondary antibody (goat anti-human IgG, HRP conjugated) was diluted 1 in 5000 with the blocking buffer. Plates were washed 3x with blocking buffer, followed by secondary antibody incubation for another 1 hour at 37° C. After the secondary antibody incubation, plates were washed 3x with washing buffer and incubated with 100 μL of ELISA chemiluminescent substrate (ThermoFisher, Catalog #37070) for 2 minutes at room temperature. The luminescence signal was detected by SpectraMax i3x at 425 nm.

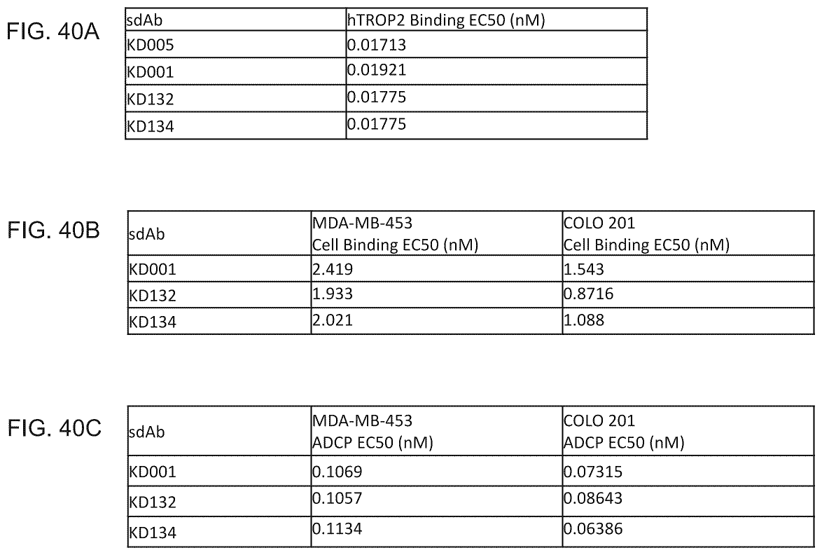

As illustrated in FIG. 40A, all variants of KD001 (KD132, KD134) showed similar binding to human TROP2 recombinant protein (EC50 values are provided in FIG. 40A).

### Cell Binding of KD001 Humanized Variants to MDA-MB-453 and COLO 201 Cells

The binding of KD001 variants (KD132, KD134) to MDA-MB-453 and COLO 201 cells was assessed by flow cytometry. In brief, 1×10≡cells were incubated with an Fc receptor 10 blocking antibody (1/100 dilution) for 10 minutes at room temperature. Anti-TROP2 antibodies were diluted in FACS staining buffer at a starting concentration of 60 nM (4.8 μg/mL) followed by 3-fold dilutions with 8 gradients, to 0.027 nM, and incubated with cells for 30 minutes on ice. After washing three times with FACS staining buffer, APC-conjugated secondary antibodies recognizing the human Fc region (Biolegend) were mixed with cells. After staining for 30 minutes, cells were washed three times, followed by resuspension into 100 μL of 7-Amino-15 Actinomycin D (7-AAD) solution (1/100 dilution). Cells were analyzed by BD FACSCanto™ II Flow cytometer (BD Bioscience). Data were analyzed with FlowJo via gating on single cells and live cells.

As illustrated in FIG. 40B, the tested variants of KD001 (KD132, KD134) showed similar binding to MDA-MB-453 and COLO 201 cell lines (EC50 values are provided in FIG. 40B).

### ADCP Activity of KD001 Humanized Variants in MDA-MB-453 and COLO 201 Cells

The ADCP activity of anti-TROP2 sdAbs KD132 and KD134 in MDA-MB-453 and COLO 201 cell lines was assessed using Promega ADCP kits (cat #CS314906). Cells were seeded at 20,000 cells per well of 96-well plates overnight. The next day, media from wells was removed and 25 μL of assay buffer was added. Then, testing antibodies were added at initial concentrations of 5 or 20 nM followed by 9x 1:2.2 serial dilutions prepared in assay buffer. ADCP effector cells (THP-1) were thawed and diluted in assay buffer and 25 μL of cell suspension was added per well of the 96-well plates containing target cell-antibody mix. Plates were incubated for 4 hours at 37 degrees Celsius, 5% CO₂. After incubation, to measure ADCP response, an equal volume (75 μL) of Bio-Glo-NL reagent was added to the wells of 96-well plates and luminescence was recorded using SpectraMax i3x Microplate Reader at an integration time of 500 millisecond per well.

FIG. 40C showed that the tested variants of KD001 (KD132, KD134) had similar ADCP activity in MDA-MB-453 and COLO 201 cells (EC50 values are provided in FIG. 40C).

### Example 23—In Vivo Anti-Tumor Effect of KD001 Humanized Variants

The anti-tumor effect of KD001 humanized variants KD133, KD135, KD136 was tested in established tumor models in mice. NCG mice (5-6 weeks old, female) were purchased from Charles River Laboratories (St.Constant, QC). CB-17 Fox Chase SCID mice (5-6 weeks old, female) were purchased either from Charles River Laboratories (St. Constant, QC) or from UHN (Toronto, ON). Mice were housed in a pathogen-free environment at the animal facility in UHN. The animal work was conducted according to the guidelines of the Canadian Council on Animal Care (CCAC) and the Animal Use Protocol approved by the Animal Care Committee at UHN. Briefly, SCID mice or NCG mice were implanted subcutaneously with 7 or 10 million tumor cells.

Animals were randomized into experimental groups as indicated when tumor volumes reached 200-1000 mm³ and were then treated (arrows) with the anti-TROP2 sdAbs or with Isotype (negative control). Tumor volume was measured once or twice per week.

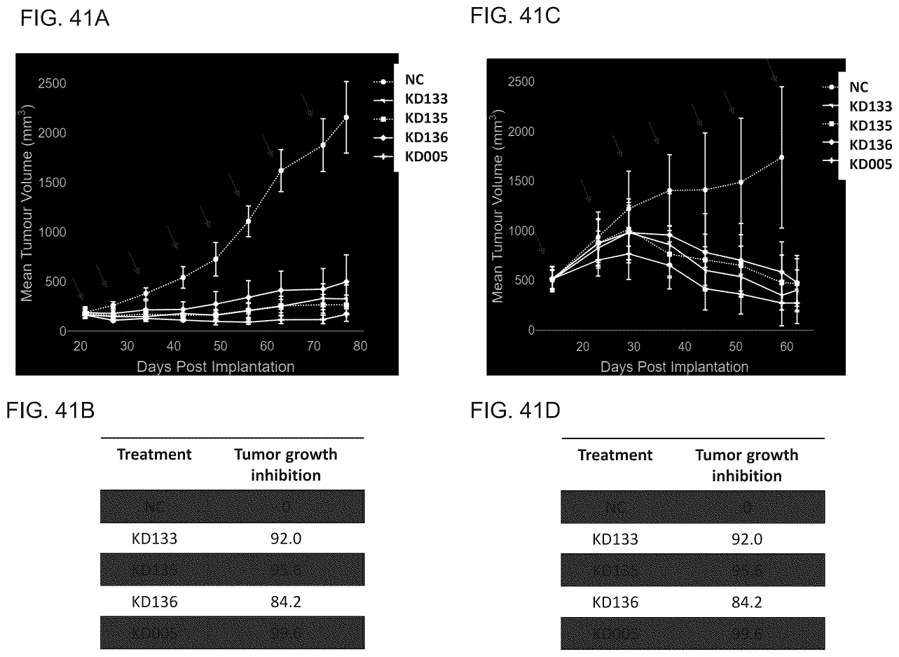

For the experiment of FIGS. 41A-41B, NCG mice (8 per group) with MDA-MB-453 tumor about 250 mm³ were treated with KD133, KD135, KD136, KD005 (positive control) or with Isotype (negative control) at 8 mg/kg, once per week for 8 doses. As illustrated in FIG. 41A and in the Table of FIG. 41B, each of KD133, KD135 and KD136 induced tumor regression compared to isotype treatment in the NCG mice MDA-MB-453 tumor model.

For the experiment of FIGS. 41C-41D, SCID mice (7 per group) with COLO201 tumor about 500 mm³ were treated with KD133, KD135, KD136, KD005 (positive control) or with Isotype (negative control) at 8 mg/kg, once per week for 7 doses. As illustrated in FIG. 41C and in the Table of FIG. 41D, each of KD133, KD135 and KD136 induced tumor regression compared to isotype treatment in the SCID mice COLO201 tumor model.

### Example 24—Binding and Activity of Variants KD137, KD138 and KD139

### Binding of Variants to Recombinant Human TROP2 Protein

Human TROP2 recombinant protein was coated on 96-well ELISA plates at the concentration of 1 g/mL. Plates were covered with adhesive plate cover and stored at 4° C. overnight. The next day, plates were washed 3x with washing buffer (PBS with 0.1% Tween™) Plates were blocked with 2% fat-free BSA in washing buffer for 1 hour at 37° C. During the incubation time, anti-TROP2 sdAbs (KD005, KD137, KD138, KD139) were diluted in the blocking buffer at a starting concentration of 200 nM followed by serial dilution 1 in 5 to 0.00256 nM. After blocking, the blocking buffer was removed from the wells and 100 μL of anti-TROP2 sdAbs dilutions were transferred to the corresponding wells. Plates were covered with adhesive plates cover and incubated at 37° C. for 1 hour. During the primary antibody incubation, the secondary antibody (goat anti-human IgG, HRP conjugated) was diluted 1 in 5000 with the blocking buffer. Plates were washed 3x with blocking buffer, followed by secondary antibody incubation for another 1 hour at 37° C. After the secondary antibody incubation, plates were washed 3x with washing buffer and added with 100 μL of ELISA chemiluminescent substrate (ThermoFisher, Catalog #37070). The luminescence signal was detected by SpectraMax i3x at 425 nm.

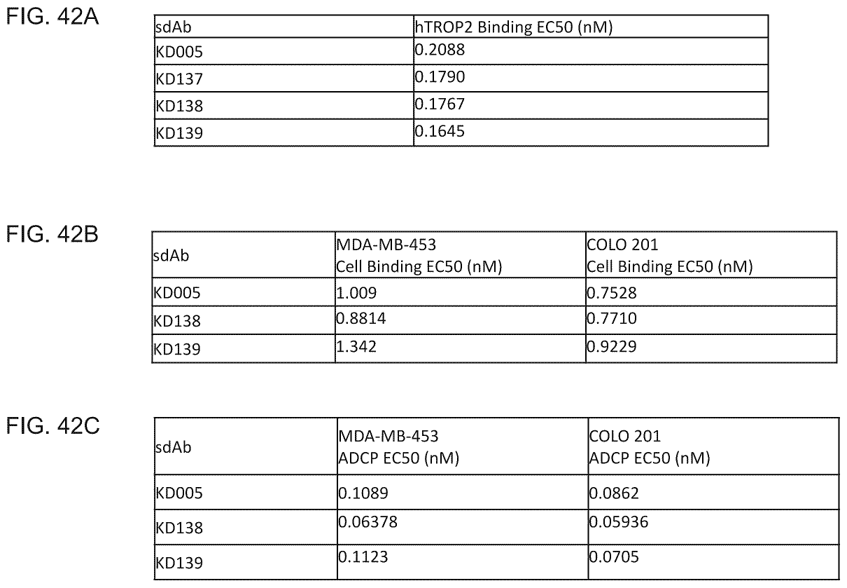

As illustrated in FIG. 42A, all tested anti-TROP2 sdAbs (KD137, KD138, KD139) showed similar binding to human TROP2 recombinant protein (EC50 values are provided in FIG. 42A).

### Cell Binding of Variants to MDA-MB-453 and COLO 201 Cells

The binding of KD005, KD138 and KD139 to MDA-MB-453 and COLO 201 cells was assessed by flow cytometry. In brief, 1×10\hat{ }5 cells were incubated with an Fc receptor 10 blocking antibody (1/100 dilution) for 10 minutes at room temperature. Anti-TROP2 antibodies were diluted in FACS staining buffer at a starting concentration of 60 nM (4.8 μg/mL) followed by 3-fold dilutions with 8 gradients, to 0.027 nM, and incubated with cells for 30 minutes on ice. After washing three times with FACS staining buffer, APC-conjugated secondary antibodies recognizing the human Fc region (Biolegend) were mixed with cells. After staining for 30 minutes, cells were washed three times, followed by resuspension into 100 μL of 7-Amino-15 Actinomycin D (7-AAD) solution (1/100 dilution). Cells were analyzed by BD FACSCanto™ II Flow cytometer (BD Bioscience). Data were analyzed with FlowJo via gating on single cells and live cells.

As illustrated in FIG. 42B, anti-TROP2 sdAbs KD005, KD138 and KD139 showed similar binding to MDA-MB-453 and COLO 201 cell lines (EC50 values are provided in FIG. 42B).

### ADCP Activity of Variants in MDA-MB-453 and COLO 201 Cells

The ADCP activity of anti-TROP2 sdAbs KD005, KD138 and KD139 in MDA-MB-453 and COLO 201 cell lines was assessed using Promega ADCP kits (cat #CS314906). Cells were seeded at 20,000 cells per well of 96-well plates overnight. The next day, media from wells was removed and 25 μL of assay buffer was added. Then, testing antibodies were added at initial concentrations of 5 or 20 nM followed by 9x 1:2.2 serial dilutions prepared in assay buffer. ADCP effector cells (THP-1) were thawed and diluted in assay buffer and 25 μL of cell suspension was added per well of the 96-well plates containing target cell-antibody mix. Plates were incubated for 4 hours at 37 degrees Celsius, 5% CO₂. After incubation, to measure ADCP response, an equal volume (75 μL) of Bio-Glo-NL reagent was added to the wells of 96-well plates and luminescence was recorded using SpectraMax i3x Microplate Reader at an integration time of 500 millisecond per well.

FIG. 42C showed that anti-TROP2 sdAbs, KD005, KD138 and KD139 showed similar ADCP activity in MDA-MB-453 and COLO 201 cells (EC50 values are provided in FIG. 42C).

### Example 25—In Vivo Anti-Tumor Effect of Variants KD137, KD138 and KD139

The anti-tumor effect of the KD137, KD138 and KD139 variants was carried out as outlined in Example 23.

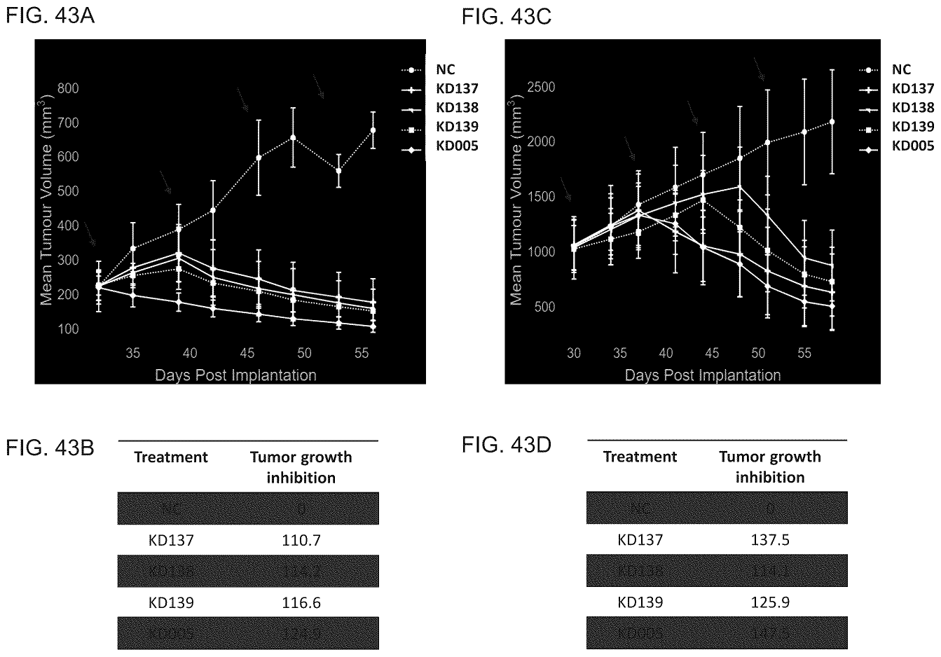

For the experiment of FIGS. 43A-43B, NCG mice (5 per group) with MDA-MB-453 tumor about 250 mm³ were treated with KD137, KD138, KD139 and KD005 (positive control) or with Isotype (negative control) at 8 mg/kg, once per week for 4 doses. As illustrated in FIG. 43A and in the Table of FIG. 43B, each of KD137, KD138 and KD139 induced tumor regression compared to isotype treatment in the NCG mice MDA-MB-453 tumor model.

For the experiment of FIGS. 43C-43D, SCID mice (5 per group) with COLO201 tumor about 1000 mm³ were treated with KD137, KD138, KD139 and KD005 (positive control) or with Isotype (negative control) at 8 mg/kg, once per week for 4 doses. As illustrated in FIG. 43C and in the Table of FIG. 43D, each of KD137, KD138 and KD139 induced tumor regression compared to isotype treatment in the SCID mice COLO201 tumor model.

### Example 26—Binding of Variants KD101 and KD076 to Human TROP2

The binding of trispecific binding agent was tested against recombinant human TROP2 or against cells expressing human TROP2.

KD142 comprises the anti-TROP2 variable region KD001 having the amino acid sequence set forth in SEQ ID NO:373 as well as an anti-PD-1 variable region having the amino acid sequence set forth in SEQ ID NO:538 and an anti-CD47 variable region having the amino acid sequence set forth in SEQ ID NO:155.

KD143 comprises the anti-TROP2 variable region KD076 having the amino acid sequence set forth in SEQ ID NO:232 as well as an anti-PD-1 variable region having the amino acid sequence set forth in SEQ ID NO:538 and an anti-CD47 variable region having the amino acid sequence set forth in SEQ ID NO:155.

In both KD142 and KD143, the anti-PD-1 and anti-CD47 variable regions are at the C-terminus of the dimerization domain and are separated by linkers. The trispecific binding agents have in a N- to C-terminal fashion, the general formula: a) antigen binding domain, b) linker, c) a dimerization domain, d) linker e) antigen binding domain, f) linker and g) antigen binding domain and therefor assembles to form a hexavalent homodimer.

### Binding of KD142 and KD143 to Recombinant Human TROP2 Protein

Human TROP2 recombinant protein was coated on 96-well ELISA plates at the concentration of 1 g/mL. Plates were covered with adhesive plate cover and stored at 4° C. overnight. The next day, plates were washed 3x with washing buffer (PBS with 0.1% Tween™ 20). Plates were blocked with 2% fat-free BSA in washing buffer for 1 hour at 37° C. During the incubation time, KD142 and KD143 antibodies were diluted in the blocking buffer to the indicated concentrations. In brief, the anti-TROP2 sdAbs starting concentration was 200 nM and serial diluted 1 in 5 to 0.00256 nM. After blocking, the blocking buffer was removed from the wells and 100 μL of anti-TROP2 sdAbs dilutions were transferred to the corresponding wells. Plates were covered with adhesive plates cover and incubated at 37° C. for 1 hour. During the primary antibody incubation, the secondary antibody (goat anti-human IgG, HRP conjugated) was diluted 1 in 5000 with the blocking buffer. Plates were washed 3x with blocking buffer, followed by secondary antibody incubation for another 1 hour at 37° C. After the secondary antibody incubation, plates were washed 3x with washing buffer and added with 100 μL of ELISA chemiluminescent substrate (ThermoFisher, Catalog #37070). The luminescence signal was detected by SpectraMax i3x at 425 nm.

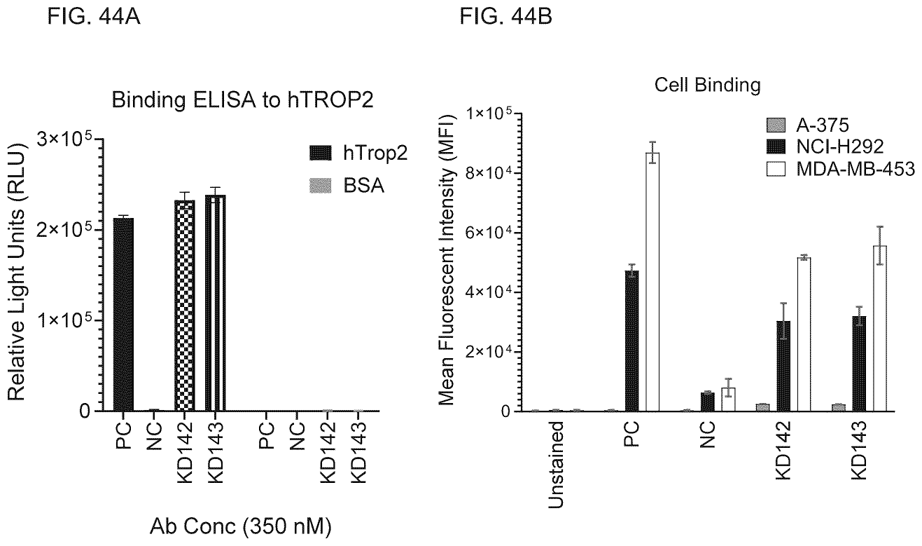

As illustrated in in FIG. 44A, KD142 and KD143 anti-TROP2 antibodies showed similar binding to human TROP2 recombinant protein. The binding of KD142 and KD143 was tested against Rhesus TROP2 recombinant protein and showed similar binding (data not shown). KD142 and KD143 showed lower binding to Rat and Mouse TROP2 recombinant proteins (data not shown).

### Cell Binding of KD142 and KD143 to MDA-MB-453, NCI-H292 and A-375 Cell Lines

The binding of KD142 and KD143 to MDA-MB-453, NCI-H292 and A-375 cells was assessed by flow cytometry. In brief, 1×10⁵ cells were incubated with an Fc receptor blocking antibody (1/100 dilution) for 10 minutes at room temperature. Anti-TROP2 antibodies were diluted in FACS staining buffer to 100 nM and added and incubated with cells for 30 minutes on ice. After washing three times with FACS staining buffer, APC-conjugated secondary antibodies recognizing the human Fc region (Biolegend) were mixed with cells. After staining for 30 minutes, cells were washed three times, followed by resuspension into 100 μL of 7-Amino-15 Actinomycin D (7-AAD) solution (1/100 dilution). Cells were analyzed by BD FACSCanto™ II Flow cytometer (BD Bioscience). Data were analyzed with FlowJo via gating on single cells and live cells.

As illustrated in FIG. 44B, anti-TROP2 antibodies, KD142 and KD143, showed higher binding to MDA-MB-453 cells compared to NCI-H292. These antibodies did not bind the TROP2 negative cell line, A-375.

In addition to the embodiments described and provided in this disclosure, the following non-limiting embodiments are contemplated.
1. A binding agent comprising one or more antigen binding domains, wherein at least one of the one or more antigen binding domains binds to trophoblast cell surface antigen-2 (TROP2) and comprises:
   a. A heavy chain complementarity determining region 1 (CDRH1) having the amino acid sequence set forth in SEQ ID NO:225, a heavy chain complementarity determining region 2 (CDRH2) having the amino acid sequence set forth in SEQ ID NO:226, SEQ ID NO:234, SEQ ID NO:242, SEQ ID NO:320, SEQ ID NO: 392 and a heavy chain complementarity determining region 3 (CDRH3) having the amino acid sequence set forth in SEQ ID NO:227 or;
   b. A heavy chain complementarity determining region 1 (CDRH1) having the amino acid sequence set forth in SEQ ID NO:228, a heavy chain complementarity determining region 2 (CDRH2) having the amino acid sequence set forth in SEQ ID NO:229, SEQ ID NO:237, SEQ ID NO:245 or SEQ ID NO:323 or SEQ ID NO:395 and a heavy chain complementarity determining region 3 (CDRH3) having the amino acid sequence set forth in SEQ ID NO:230.
2. The binding agent of the preceding embodiment, wherein the at least one of the one or more antigen binding domains comprises an amino acid sequence as set forth in SEQ ID NO:539.
3. The binding agent of any of the preceding embodiments, wherein the at least one of the one or more antigen binding domains comprises an amino acid sequence as set forth in SEQ ID NO:540.
4. The binding agent of any of the preceding embodiments, wherein the at least one of the one or more antigen binding domains comprises an amino acid sequence as set forth in SEQ ID NO:541.
5. The binding agent of any of the preceding embodiments, wherein the at least one of the one or more antigen binding domains comprises an amino acid sequence as set forth in any one of SEQ ID NOs:514-516.
6. The binding agent of any of the preceding embodiments, wherein the at least one of the one or more antigen binding domains comprises an amino acid sequence as set forth in any one of SEQ ID NOs:517-519.
7. The binding agent of any of the preceding embodiments, wherein the at least one of the one or more antigen binding domains comprises an amino acid sequence as set forth in any one of SEQ ID NOs:520-522.
8. The binding agent of any of the preceding embodiments, wherein the at least one of the one or more antigen binding domains comprises an amino acid sequence as set forth in any one of SEQ ID NOs:523-525.
9. The binding agent of any of the preceding embodiments, wherein the at least one of the one or more antigen binding domains comprises an amino acid sequence as set forth in any one of SEQ ID NOs:526-528.
10. The binding agent of any of the preceding embodiments, wherein the at least one of the one or more antigen binding domains comprises an amino acid sequence at least 80% identical to the amino acid sequence set forth in SEQ ID NO:373, SEQ ID NO:232, SEQ ID NO:231, SEQ ID NO:295, SEQ ID NO:296, SEQ ID NO:297, SEQ ID NO:372, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:239, SEQ ID NO:240, SEQ ID NO:383, SEQ ID NO:384, SEQ ID NO:385, SEQ ID NO:386, SEQ ID NO:387, SEQ ID NO:247, SEQ ID NO:388, SEQ ID NO:325, SEQ ID NO:390, SEQ ID NO:397, or SEQ ID NO:398.
11. The binding agent of any of the preceding embodiments, wherein the at least one of the one or more antigen binding domains comprises an amino acid sequence at least 90% identical to the amino acid sequence set forth in SEQ ID NO:373, SEQ ID NO:232, SEQ ID NO:231, SEQ ID NO:295, SEQ ID NO:296, SEQ ID NO:297, SEQ ID NO:372, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:239, SEQ ID NO:240, SEQ ID NO:383, SEQ ID NO:384, SEQ ID NO:385, SEQ ID NO:386, SEQ ID NO:387, SEQ ID NO:247, SEQ ID NO:388, SEQ ID NO:325, SEQ ID NO:390, SEQ ID NO:397, or SEQ ID NO:398.
12. A binding agent comprising one or more antigen binding domains, wherein at least one of the one or more antigen binding domains specifically binds to trophoblast cell surface antigen-2 (TROP2) and comprises a heavy chain complementarity determining region 1 (CDRH1) having the amino acid sequence set forth in SEQ ID NO:488, a heavy chain complementarity determining region 2 (CDRH2) having the amino acid sequence set forth in SEQ ID NO:489 and a heavy chain complementarity determining region 3 (CDRH3) having the amino acid sequence set forth in SEQ ID NO:490 and an amino acid sequence at least 80% identical to the amino acid sequence set forth in any one of SEQ ID NO: 493, SEQ ID NO:496 or SEQ ID NO:491.
13. A binding agent comprising one or more antigen binding domains, wherein at least one of the one or more antigen binding domains specifically binds to trophoblast cell surface antigen-2 (TROP2) and comprises a heavy chain complementarity determining region 1 (CDRH1) having the amino acid sequence set forth in SEQ ID NO:500, a heavy chain complementarity determining region 2 (CDRH2) having the amino acid sequence set forth in SEQ ID NO:489 and a heavy chain complementarity determining region 3 (CDRH3) having the amino acid sequence set forth in SEQ ID NO:501 and an amino acid sequence at least 80% identical to the amino acid sequence set forth in any one of SEQ ID NO:503 or SEQ ID NO:502.
14. A binding agent comprising one or more antigen binding domains, wherein at least one of the one or more antigen binding domains specifically binds to trophoblast cell surface antigen-2 (TROP2) and comprises a heavy chain complementarity determining region 1 (CDRH1) having the amino acid sequence set forth in SEQ ID NO:308, a heavy chain complementarity determining region 2 (CDRH2) having the amino acid sequence set forth in SEQ ID NO:309 and a heavy chain complementarity determining region 3 (CDRH3) having the amino acid sequence set forth in SEQ ID NO:310 and an amino acid sequence at least 80% identical to the amino acid sequence set forth in any one of SEQ ID NO:311 or SEQ ID NO:389.
15. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:215, a CDRH2 having the amino acid sequence as set forth in SEQ ID NO:216 and/or a CDRH3 having the amino acid sequence as set forth in SEQ ID NO:217;
16. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:218, a CDRH2 having the amino acid sequence as set forth in SEQ ID NO:219 and/or a CDRH3 having the amino acid sequence as set forth in SEQ ID NO:220;
17. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:225, a CDRH2 having the amino acid sequence as set forth in SEQ ID NO:226 and/or a CDRH3 having the amino acid sequence as set forth in SEQ ID NO:227;
18. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:228, a CDRH2 having the amino acid sequence as set forth in SEQ ID NO:229 and/or a CDRH3 having the amino acid sequence as set forth in SEQ ID NO:230;
19. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:233, a CDRH2 having the amino acid sequence as set forth in SEQ ID NO:234 and/or a CDRH3 having the amino acid sequence as set forth in SEQ ID NO:235;
20. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:236, a CDRH2 having the amino acid sequence as set forth in SEQ ID NO:237 and/or a CDRH3 having the amino acid sequence as set forth in SEQ ID NO:238;
21. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:241, a CDRH2 having the amino acid sequence as set forth in SEQ ID NO:242 and/or a CDRH3 having the amino acid sequence as set forth in SEQ ID NO:243;
22. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:244, a CDRH2 having the amino acid sequence as set forth in SEQ ID NO:245 and/or a CDRH3 having the amino acid sequence as set forth in SEQ ID NO:246;
23. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:298, a CDRH2 having the amino acid sequence as set forth in SEQ ID NO:299 and/or a CDRH3 having the amino acid sequence as set forth in SEQ ID NO:300;
24. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:301, a CDRH2 having the amino acid sequence as set forth in SEQ ID NO:302 and/or a CDRH3 having the amino acid sequence as set forth in SEQ ID NO:303;
25. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:305, a CDRH2 having the amino acid sequence as set forth in SEQ ID NO:306 and/or a CDRH3 having the amino acid sequence as set forth in SEQ ID NO:307;
26. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:308, a CDRH2 having the amino acid sequence as set forth in SEQ ID NO:309 and/or a CDRH3 having the amino acid sequence as set forth in SEQ ID NO:310;
27. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:312, a CDRH2 having the amino acid sequence as set forth in SEQ ID NO:313 and/or a CDRH3 having the amino acid sequence as set forth in SEQ ID NO:314;
28. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:315, a CDRH2 having the amino acid sequence as set forth in SEQ ID NO:316 and/or a CDRH3 having the amino acid sequence as set forth in SEQ ID NO:317;
29. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:319, a CDRH2 having the amino acid sequence as set forth in SEQ ID NO:320 and/or a CDRH3 having the amino acid sequence as set forth in SEQ ID NO:321;
30. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:322, a CDRH2 having the amino acid sequence as set forth in SEQ ID NO:323 and/or a CDRH3 having the amino acid sequence as set forth in SEQ ID NO:324;
31. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:326, a CDRH2 having the amino acid sequence as set forth in SEQ ID NO:327 and/or a CDRH3 having the amino acid sequence as set forth in SEQ ID NO:328;
32. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:329, a CDRH2 having the amino acid sequence as set forth in SEQ ID NO:330 and/or a CDRH3 having the amino acid sequence as set forth in SEQ ID NO:331;
33. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:333, a CDRH2 having the amino acid sequence as set forth in SEQ ID NO:334 and/or a CDRH3 having the amino acid sequence as set forth in SEQ ID NO:335;
34. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:336, a CDRH2 having the amino acid sequence as set forth in SEQ ID NO:337 and/or a CDRH3 having the amino acid sequence as set forth in SEQ ID NO:338;
35. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:340, a CDRH2 having the amino acid sequence as set forth in SEQ ID NO:341 and/or a CDRH3 having the amino acid sequence as set forth in SEQ ID NO:342;
36. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:343, a CDRH2 having the amino acid sequence as set forth in SEQ ID NO:344 and/or a CDRH3 having the amino acid sequence as set forth in SEQ ID NO:345;
37. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:347, a CDRH2 having the amino acid sequence as set forth in SEQ ID NO:348 and/or a CDRH3 having the amino acid sequence as set forth in SEQ ID NO:349;
38. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:350, a CDRH2 having the amino acid sequence as set forth in SEQ ID NO:351 and/or a CDRH3 having the amino acid sequence as set forth in SEQ ID NO:352;
39. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:391, a CDRH2 having the amino acid sequence as set forth in SEQ ID NO:392 and/or a CDRH3 having the amino acid sequence as set forth in SEQ ID NO:393;
40. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:394, a CDRH2 having the amino acid sequence as set forth in SEQ ID NO:395 and/or a CDRH3 having the amino acid sequence as set forth in SEQ ID NO:396;
41. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:399, a CDRH2 having the amino acid sequence as set forth in SEQ ID NO:400 and/or a CDRH3 having the amino acid sequence as set forth in SEQ ID NO:401;
42. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:402, a CDRH2 having the amino acid sequence as set forth in SEQ ID NO:403 and/or a CDRH3 having the amino acid sequence as set forth in SEQ ID NO:404;
43. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:406, a CDRH2 having the amino acid sequence as set forth in SEQ ID NO:407 and/or a CDRH3 having the amino acid sequence as set forth in SEQ ID NO:408;
44. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:409, a CDRH2 having the amino acid sequence as set forth in SEQ ID NO:410 and/or a CDRH3 having the amino acid sequence as set forth in SEQ ID NO:411;
45. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:413, a CDRH2 having the amino acid sequence as set forth in SEQ ID NO:414 and/or a CDRH3 having the amino acid sequence as set forth in SEQ ID NO:415;
46. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:416, a CDRH2 having the amino acid sequence as set forth in SEQ ID NO:417 and/or a CDRH3 having the amino acid sequence as set forth in SEQ ID NO:418;
47. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:420, a CDRH2 having the amino acid sequence as set forth in SEQ ID NO:421 and/or a CDRH3 having the amino acid sequence as set forth in SEQ ID NO:422;
48. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:423, a CDRH2 having the amino acid sequence as set forth in SEQ ID NO:424 and/or a CDRH3 having the amino acid sequence as set forth in SEQ ID NO:425;
49. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:427, a CDRH2 having the amino acid sequence as set forth in SEQ ID NO:428 and/or a CDRH3 having the amino acid sequence as set forth in SEQ ID NO:429;
50. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:430, a CDRH2 having the amino acid sequence as set forth in SEQ ID NO:431 and/or a CDRH3 having the amino acid sequence as set forth in SEQ ID NO:432;
51. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:434, a CDRH2 having the amino acid sequence as set forth in SEQ ID NO:435 and/or a CDRH3 having the amino acid sequence as set forth in SEQ ID NO:436;
52. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:437, a CDRH2 having the amino acid sequence as set forth in SEQ ID NO:438 and/or a CDRH3 having the amino acid sequence as set forth in SEQ ID NO:439;
53. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:441, a CDRH2 having the amino acid sequence as set forth in SEQ ID NO:442 and/or a CDRH3 having the amino acid sequence as set forth in SEQ ID NO:443;
54. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:444, a CDRH2 having the amino acid sequence as set forth in SEQ ID NO:445 and/or a CDRH3 having the amino acid sequence as set forth in SEQ ID NO:446;
55. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:448, a CDRH2 having the amino acid sequence as set forth in SEQ ID NO:449 and/or a CDRH3 having the amino acid sequence as set forth in SEQ ID NO:450;
56. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:451, a CDRH2 having the amino acid sequence as set forth in SEQ ID NO:452 and/or a CDRH3 having the amino acid sequence as set forth in SEQ ID NO:453;
57. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:455, a CDRH2 having the amino acid sequence as set forth in SEQ ID NO:456 and/or a CDRH3 having the amino acid sequence as set forth in SEQ ID NO:457;
58. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:458, a CDRH2 having the amino acid sequence as set forth in SEQ ID NO:459 and/or a CDRH3 having the amino acid sequence as set forth in SEQ ID NO:460;
59. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:462, a CDRH2 having the amino acid sequence as set forth in SEQ ID NO:463 and/or a CDRH3 having the amino acid sequence as set forth in SEQ ID NO:464, or;
60. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:465, a CDRH2 having the amino acid sequence as set forth in SEQ ID NO:466 and/or a CDRH3 having the amino acid sequence as set forth in SEQ ID NO:467.
61. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:485, a CDRH2 having the amino acid sequence as set forth in SEQ ID NO:486 and/or a CDRH3 having the amino acid sequence as set forth in SEQ ID NO:487.
62. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:488, a CDRH2 having the amino acid sequence as set forth in SEQ ID NO:489 and/or a CDRH3 having the amino acid sequence as set forth in SEQ ID NO:490.
63. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:485, a CDRH2 having the amino acid sequence as set forth in SEQ ID NO:492 and/or a CDRH3 having the amino acid sequence as set forth in SEQ ID NO:487.
64. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:485, a CDRH2 having the amino acid sequence as set forth in SEQ ID NO:495 and/or a CDRH3 having the amino acid sequence as set forth in SEQ ID NO:487.
65. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:485, a CDRH2 having the amino acid sequence as set forth in SEQ ID NO:498 and/or a CDRH3 having the amino acid sequence as set forth in SEQ ID NO:487.
66. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:500, a CDRH2 having the amino acid sequence as set forth in SEQ ID NO:489 and/or a CDRH3 having the amino acid sequence as set forth in SEQ ID NO:501.
67. A binding agent comprising one or more antigen binding domains, wherein at least one of the one or more antigen binding domains specifically binds to trophoblast cell surface antigen-2 (TROP2) and comprises:
   a CDRH1 having the amino acid sequence as set forth in SEQ ID NO:485, a CDRH2 having the amino acid sequence as set forth in SEQ ID NO:486 and/or a CDRH3 having the amino acid sequence as set forth in SEQ ID NO:499.
68. The binding agent of any of the preceding embodiments, wherein the one or more antigen binding domains is one or more heavy chain variable regions.
69. The binding agent of any of the preceding embodiments, wherein the one or more heavy chain variable regions are partially or fully humanized.
70. The binding agent of any of the preceding embodiments, wherein the one or more heavy chain variable regions comprise human or humanized framework regions.
71. The binding agent of any of the preceding embodiments, wherein the at least one of the one or more antigen binding domains comprises the humanized heavy chain variable region backbone comprising the amino acid sequence set forth in the amino acid sequence set forth in SEQ ID NO:211, SEQ ID NO:477 or SEQ ID NO:478.
72. The binding agent of any of the preceding embodiments, wherein the at least one of the one or more antigen binding domains comprises the humanized heavy chain variable region backbone comprising the amino acid sequence set forth in the amino acid sequence set forth in SEQ ID NO:213, SEQ ID NO:479 or SEQ ID NO:470.
73. The binding agent of any of the preceding embodiments, wherein the one or more heavy chain variable regions comprise an amino acid sequence at least 80% identical, at least 85% identical, at least 90% identical, at least 95% identical, at least 99% identical or identical to the amino acid sequence set forth in SEQ ID NO:221, SEQ ID NO:222, SEQ ID NO:223, SEQ ID NO:224, SEQ ID NO:231, SEQ ID NO:232, SEQ ID NO:239, SEQ ID NO:240, SEQ ID NO:247, SEQ ID NO:295, SEQ ID NO:296, SEQ ID NO:297, SEQ ID NO:304, SEQ ID NO:311, SEQ ID NO:318, SEQ ID NO:325, SEQ ID NO: 332, SEQ ID NO:339, SEQ ID NO:346, SEQ ID NO:353, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503.
74. The binding agent of any of the preceding embodiments, wherein the one or more heavy chain variable regions comprise amino acid substitutions, additions or deletions in the amino acid sequence set forth in SEQ ID NO:221, SEQ ID NO:222, SEQ ID NO:223, SEQ ID NO:224, SEQ ID NO:231, SEQ ID NO:232, SEQ ID NO:239, SEQ ID NO:240, SEQ ID NO:247, SEQ ID NO:295, SEQ ID NO:296, SEQ ID NO:297, SEQ ID NO:304, SEQ ID NO:311, SEQ ID NO:318, SEQ ID NO:325, SEQ ID NO: 332, SEQ ID NO:339, SEQ ID NO:346, SEQ ID NO:353, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503.
75. The binding agent of any of the preceding embodiments, wherein the amino acid substitution is at a position selected from position 1, position 5, position 11, position 12, position 13, position 14, position 44, position 45, position 50, position 60, position 62, position 74, position 81, position 84, position 86, position 87, position 88, position 92, position 94, position 115 or combination thereof.
76. The binding agent of any of the preceding embodiments, wherein the amino acid substitution is at a position selected from position 1, position 5, position 11, position 14, position 44, position 45, position 50, position 60, position 62, position 74, position 81, position 84, position 86, position 87, position 92, position 94, position 115 or combination thereof.
77. The binding agent of any of the preceding embodiments, wherein the amino acid substitution is at a position selected from position 1, position 5, position 12, position 13, position 14, position 45, position 50, position 60, position 62, position 74, position 86, position 87, position 88, position 92, position 94, position 115 or combination thereof.
78. The binding agent of any of the preceding embodiments, wherein the one or more heavy chain variable regions comprise amino acid substitutions in the amino acid sequence set forth in SEQ ID NO:221, SEQ ID NO:222, SEQ ID NO:223, SEQ ID NO:224, SEQ ID NO:231, SEQ ID NO:232, SEQ ID NO:239, SEQ ID NO:240, SEQ ID NO:247, SEQ ID NO:295, SEQ ID NO:296, SEQ ID NO:297, SEQ ID NO:304, SEQ ID NO:311, SEQ ID NO:318, SEQ ID NO:325, SEQ ID NO: 332, SEQ ID NO:339, SEQ ID NO:346, SEQ ID NO:353, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503 and wherein the amino acid substitutions are selected from:
   t) E at position 1, V at position 5, L at position 11, P at position 14, A at position 60, T at position 62, Q at position 81, S at position 84, R at position 86, A at position 87, V at position 92, Y at position 94,
   u) V at position 5, L at position 11, P at position 14, T at position 62, Q at position 81, S at position 84, R at position 86, A at position 87, V at position 92, Y at position 94,
   v) V at position 5, L at position 11, P at position 14, T at position 62, Q at position 81, S at position 84, R at position 86, A at position 87, V at position 92,
   w) V at position 5, L at position 11, T at position 62, Q at position 81, S at position 84, V at position 92,
   x) T at position 62, Q at position 81, S at position 84, V at position 92, y) Q at position 81, S at position 84, V at position 92,
   z) E at position 1, L at position 5, L at position 11, P at position 14, L at position 45, V at position 50, A at position 60, T at position 62, S at position 74, Q at position 81, S at position 84, R at position 86, A at position 87, V at position 92, Y at position 94, M at position 115,
   aa) L at position 11, P at position 14, T at position 62, S at position 74, Q at position 81, R at position 86, A at position 87, M at position 115,
   bb) L at position 11, P at position 14, L at position 45, A at position 60, T at position 62, S at position 74, Q at position 81, S at position 84, R at position 86, A at position 87, V at position 92, M at position 115,
   cc) E at position 1, L at position 11, P at position 14, L at position 45, A at position 60, T at position 62, S at position 74, Q at position 81, S at position 84, R at position 86, A at position 87, V at position 92, M at position 115,
   dd) L at position 5, L at position 11, P at position 14, L at position 45, A at position 60, T at position 62, S at position 74, Q at position 81, S at position 84, R at position 86, A at position 87, V at position 92, M at position 115,
   ee) L at position 11, P at position 14, L at position 45, V at position 50, A at position 60, T at position 62, S at position 74, Q at position 81, S at position 84, R at position 86, A at position 87, V at position 92, M at position 115,
   ff) L at position 11, P at position 14, L at position 45, A at position 60, T at position 62, S at position 74, Q at position 81, S at position 84, R at position 86, A at position 87, V at position 92, Y at position 94, M at position 115,
   gg) L at position 11, P at position 14, A at position 60, T at position 62, S at position 74, Q at position 81, S at position 84, R at position 86, A at position 87, V at position 92, M at position 115,
   hh) L at position 11, P at position 14, L at position 45, T at position 62, S at position 74, Q at position 81, S at position 84, R at position 86, A at position 87, V at position 92, M at position 115,
   ii) L at position 11, P at position 14, L at position 45, A at position 60, T at position 62, S at position 74, Q at position 81, R at position 86, A at position 87, V at position 92, M at position 115,
   jj) L at position 11, P at position 14, L at position 45, A at position 60, T at position 62, S at position 74, Q at position 81, S at position 84, R at position 86, A at position 87, M at position 115,
   kk) P at position 14, L at position 45, A at position 60, T at position 62, S at position 74, Q at position 81, S at position 84, R at position 86, A at position 87, V at position 92 or,
   ll) L at position 11, P at position 14, Q at position 44, R at position 45, A at position 60, T at position 62, S at position 74, Q at position 81, S at position 84, R at position 86, A at position 87, V at position 92, M at position 115.
79. The binding agent of any of the preceding embodiments, wherein the one or more heavy chain variable regions comprise amino acid substitutions in the amino acid sequence set forth in SEQ ID NO:221, SEQ ID NO:222, SEQ ID NO:223, SEQ ID NO:224, SEQ ID NO:231, SEQ ID NO:232, SEQ ID NO:239, SEQ ID NO:240, SEQ ID NO:247, SEQ ID NO:295, SEQ ID NO:296, SEQ ID NO:297, SEQ ID NO:304, SEQ ID NO:311, SEQ ID NO:318, SEQ ID NO:325, SEQ ID NO: 332, SEQ ID NO:339, SEQ ID NO:346, SEQ ID NO:353, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:397, SEQ ID NO:398, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461 or SEQ ID NO:468, SEQ ID NO:469, SEQ ID NO:470, SEQ ID NO:471, SEQ ID NO:472, SEQ ID NO:473, SEQ ID NO:491, SEQ ID NO:493, SEQ ID NO:496, SEQ ID NO:502, or SEQ ID NO:503 and wherein the amino acid substitutions are selected from:
   a. E at position 1, L at position 5, Q at position, 13 P at position 14, L at position 45, A at position 50, A at position 60, T at position 62, S at position 74, R at position 86, A at position 87, E at position 88, V at position 92, Y at position 94, L at position 115,
   b. E at position 1, L at position 15, Q at position 13, P at position 14, L at position 45, A at position 60, T at position 62, S at position 74, R at position 86, A at position 87, E at position 88, V at position 92, Y at position 94, L at position 115,
   c. E at position 1, L at position 5, Q at position 13, P at position 14, L at position 45, A at position 60, T at position 62, S at position 74, R at position 86, A at position 87, E at position 88, V at position 92, L at position 115,
   d. L at position 5, Q at position 13, P at position 14, A at position 60, T at position 62, S at position 74, R at position 86, A at position 87, E at position 88, V at position 92, Y at position 94, L at position 115,
   e. L at position 5, Q at position 13, P at position 14, A at position 60, T at position 62, S at position 74, R at position 86, A at position 87, E at position 88, V at position 92, L at position 115,
   f. Q at position 13, P at position 14, T at position 62, S at position 74, R at position 86, A at position 87, E at position 88, V at position 92, Y at position 94,
   g. Q at position 13, P at position 14, T at position 62, S at position 74, R at position 86, A at position 87, E at position 88, V at position 92,
   h. Q at position 13, P at position 14, T at position 62, S at position 74, V at position 92,
   i. Q at position 13, P at position 14, T at position 62,
   j. E at position 1, V at position 5, I at position 12, Q at position 13, P at position 14, L at position 45, V at position 50, A at position 60, T at position 62, S at position 74, R at position 86, A at position 87, E at position 88, V at position 92, Y at position 94, L at position 115,
   k. I at position 12, Q at position 13, P at position 14, T at position 62, S at position 74, R at position 86, A at position 87, L at position 115,
   l. I at position 12, Q at position 13, P at position 14, L at position 45, A at position 60, T at position 62, S at position 74, R at position 86, A at position 87, E at position 88, V at position 92, L at position 115,
   m. E at position 1, I at position 12, Q at position 13, P at position 14, L at position 45, A at position 60, T at position 62, S at position 74, R at position 86, A at position 87, E at position 88, V at position 92, L at position 115,
   n. V at position 5, I at position 12, Q at position 13, P at position 14, L at position 45, A at position 60, T at position 62, S at position 74, R at position 86, A at position 87, E at position 88, V at position 92, L at position 115,
   o. I at position 12, Q at position 13, P at position 14, L at position 45, V at position 50, A at position 60, T at position 62, S at position 74, R at position 86, A at position 87, E at position 88, V at position 92, L at position 115,
   p. I at position 12, Q at position 13, P at position 14, L at position 45, A at position 60, T at position 62, S at position 74, R at position 86, A at position 87, E at position 88, V at position 92, Y at position 94, L at position 115,
   q. I at position 12, Q at position 13, P at position 14, A at position 60, T at position 62, S at position 74, R at position 86, A at position 87, E at position 88, V at position 92, L at position 115,
   r. I at position 12, Q at position 13, P at position 14, L at position 45, T at position 62, S at position 74, R at position 86, A at position 87, E at position 88, V at position 92, L at position 115,
   s. I at position 12, Q at position 13, P at position 14, L at position 45, A at position 60, T at position 62, S at position 74, R at position 86, A at position 87, V at position 92, L at position 115,
   t. I at position 12, Q at position 13, P at position 14, L at position 45, A at position 60, T at position 62, S at position 74, R at position 86, A at position 87, E at position 88, L at position 115 or,
   u. I at position 12, P at position 14, L at position 45, A at position 60, T at position 62, S at position 74, R at position 86, A at position 87, E at position 88, V at position 92.
80. The binding agent of any of the preceding embodiments, wherein the at least one of the one or more antigen binding domains comprises the humanized heavy chain variable region backbone comprising the amino acid sequence set forth in SEQ ID NO: 248, SEQ ID NO:249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, 266, SEQ ID NO: 267, SEQ ID NO: 268, SEQ ID NO: 269, SEQ ID NO: 270 or SEQ ID NO: 271 or comprising from one to five amino acid substitutions, additions and/or deletions thereto.
81. The binding agent of any of the preceding embodiments, wherein the at least one of the one or more antigen binding domains comprises the humanized heavy chain variable region backbone comprising the amino acid sequence set forth in SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293 and SEQ ID NO: 294 or comprising from one to five amino acid substitutions, additions and/or deletions thereto.
82. The binding agent of any of the preceding embodiments, wherein the one or more heavy chain variable regions comprise an amino acid sequence as set forth in SEQ ID NO:221, SEQ ID NO:222, SEQ ID NO:223, SEQ ID NO:224, SEQ ID NO:231, SEQ ID NO:232, SEQ ID NO:239, SEQ ID NO:240, SEQ ID NO:247, SEQ ID NO:295, SEQ ID NO:296, SEQ ID NO:297, SEQ ID NO:304, SEQ ID NO:311, SEQ ID NO:318, SEQ ID NO:325, SEQ ID NO: 332, SEQ ID NO:339, SEQ ID NO:346, SEQ ID NO:353 or comprises from one to five amino acid substitution, addition and/or deletion in said amino acid sequence thereof.
83. The binding agent of any of the preceding embodiments, wherein the amino acid sequence of the one or more heavy chain variable region comprises amino acid residue V at position 37, amino acid residue Q at position 39, amino acid residue P at position 45, amino acid residue W at position 47, amino acid residue Y at position 50, amino acid residue Y at position 58, amino acid residue R at position 99, amino acid residue L at position 100, amino acid residue T at position 101, amino acid residue D at position 102, amino acid residue S at position 103, amino acid residue H at position 104 i, amino acid residue Y at position 105, amino acid residue V at position 106, amino acid residue E at position 107, amino acid residue D at position 108, amino acid residue A at position 109 or combination thereof.
84. The binding agent of any of the preceding embodiments, wherein the amino acid sequence of the one or more heavy chain variable regions comprises amino acid residue Q at position 1, amino acid residue V at position 2, amino acid residue V at position 37, amino acid residue Q at position 39, amino acid residue P at position 45, amino acid residue W at position 47, amino acid residue Y at position 50, amino acid residue Y at position 58, amino acid residue R at position 99, amino acid residue L at position 100, amino acid residue T at position 101, amino acid residue D at position 102, amino acid residue S at position 103, amino acid residue H at position 104, amino acid residue Y at position 105, amino acid residue V at position 106, amino acid residue E at position 107, amino acid residue D at position 108, amino acid residue A at position 109, amino acid residue W at position 110, amino acid residue Q at position 112 or combination thereof.
85. The binding agent of any of the preceding embodiments, wherein the amino acid sequence of the heavy chain variable regions comprises amino acid residue Q at position 1 Q, amino acid residue V at position 2, amino acid residue Q at position 3, amino acid residue L at position 4, amino acid residue V at position 37 is V, amino acid residue Q at position 39, amino acid residue P at position 45, amino acid residue E at position 46, amino acid residue W at position 47, amino acid residue Y at position 50, amino acid residue Y at position 58, amino acid residue R at position 99, amino acid residue L at position 100, amino acid residue T at position 101, amino acid residue D at position 102, amino acid residue S at position 103, amino acid residue H at position 104, amino acid residue Y at position 105, amino acid residue V at position 106, amino acid residue E at position 107, amino acid residue D at position 108, amino acid residue A at position 109, amino acid residue W at position 110, amino acid residue G at position 111, amino acid residue Q at position 112 is Q or combination thereof.
86. The binding agent of any of the preceding embodiments wherein the amino acid substitution is outside a CDR sequence.
87. The binding agent of any of the preceding embodiments, wherein the amino acid substitution is outside a CDR3 sequence.
88. The binding agent of any of the preceding embodiments, wherein the amino acid substitution is outside framework region 2 (FR2) sequence.
89. The binding agent of any of the preceding embodiments, wherein the heavy chain variable region framework region 2 (FR2) comprises the sequence set forth in SEQ ID NO:116, SEQ ID NO:117, SEQ ID NO:118, SEQ ID NO:119, SEQ ID NO:120, SEQ ID NO:354, SEQ ID NO:355, SEQ ID NO:356, SEQ ID NO:360, SEQ ID NO:361 or SEQ ID NO:505.
90. The binding agent of any of the preceding embodiments, wherein the FR2 sequence is as set forth in SEQ ID NO:116.
91. The binding agent of any of the preceding embodiments, wherein the one or more heavy chain variable regions are from one or more single domain antibodies.
92. The binding agent of any of the preceding embodiments, wherein the one or more heavy chain variable regions are from a camelid VH.
93. The binding agent of any of the preceding embodiments, wherein the one or more heavy chain variable regions are from a camelid VHH.
94. The binding agent of any of the preceding embodiments, wherein the binding agent comprises two antigen binding domains or more.
95. The binding agent of any of the preceding embodiments, wherein the binding agent is monospecific.
96. The binding agent of any of the preceding embodiments, wherein the binding agent is bispecific.
97. The binding agent of any of the preceding embodiments, wherein the binding agent is multispecific.
98. The binding agent of any of the preceding embodiments, wherein the binding agent comprises one or more antigen binding domain that binds to CD47 and/or one or more antigen binding domain that binds to PD-1.
99. The binding agent of any of the preceding embodiments, wherein the binding agent comprises two polypeptide chains and wherein each polypeptide chain comprises in a N- to C-terminal fashion, a) the one or more antigen binding domains, b) optionally a linker and c) a dimerization domain.
100. The binding agent of any of the preceding embodiments, wherein the binding agent comprises two polypeptide chains and wherein each polypeptide chain comprises in a N- to C-terminal fashion an amino acid sequence of formula I:

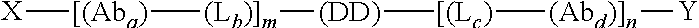

Wherein m is 0, 1, 2 or an integer greater than 2;
Wherein n is 0, 1, 2 or an integer greater than 2;
Wherein m and n are not 0 simultaneously;
Wherein Abₐ, Ab_{d}, each represents an antigen binding domain wherein at least one of the antigen binding domains is an antigen binding domain 1 (ABD1), an antigen binding domain 2 (ABD2) or an antigen binding domain 3 (ABD3);
Wherein X or Y are independently present or absent and comprises an amino acid sequence;
Wherein L_{b}, L_{c}, each independently comprises one or more linkers; and
Wherein DD represents a dimerization domain
101. The binding agent of any of the preceding embodiments, wherein the binding agent comprises a single domain antibody.
102. The binding agent of any of the preceding embodiments, wherein the binding agent is a single domain antibody.
103. The binding agent of any of the preceding embodiments, wherein the binding agent comprises a dimerization domain.
104. The binding agent of any of the preceding embodiments, wherein the dimerization domain comprises a CH3 domain of an antibody or a portion thereof.
105. The binding agent of any of the preceding embodiments, wherein the CH3 domain is a natural CH3 domain or a mutated CH3 domain.
106. The binding agent of any of the preceding embodiments, wherein the CH3 domain is from an IgG1, an IgG2, an IgG3 or an IgG4 heavy chain.
107. The binding agent of any of the preceding embodiments, wherein the CH3 domain is from a human IgG1, human IgG2, human IgG3 or human IgG4 heavy chain.
108. The binding agent of any of the preceding embodiments, wherein the dimerization domain comprises a CH2 domain of an antibody or a portion thereof.
109. The binding agent of any of the preceding embodiments, wherein the CH2 domain is a natural CH2 domain or a mutated CH2 domain.
110. The binding agent of any of the preceding embodiments, wherein the CH2 domain is from an IgG1, an IgG2, an IgG3 or an IgG4 heavy chain.
111. The binding agent of any of the preceding embodiments, wherein the CH2 domain is from a human IgG1, human IgG2, human IgG3 or human IgG4 heavy chain.
112. The binding agent of any of the preceding embodiments, wherein the dimerization domain also comprises a hinge and/or spacer.
113. The binding agent of any of the preceding embodiments, wherein the dimerization domain comprises an amino acid sequence at least 80% identical to the amino acid sequence set forth in SEQ ID NO:212.
114. The binding agent of any of the preceding embodiments, wherein the dimerization domain comprises an amino acid sequence at least 80% identical to the amino acid sequence set forth in SEQ ID NO:506.
115. The binding agent of any of the preceding embodiments, wherein the dimerization domain comprises an amino acid sequence at least 80% identical to the amino acid sequence set forth in SEQ ID NO:214.
116. The binding agent of any of the preceding embodiments, wherein the binding agent comprises at least two antigen binding domains.
117. The binding agent of any of the preceding embodiments, wherein the binding agent comprises at least two identical antigen binding domains.
118. The binding agent of any of the preceding embodiments, wherein each of the two antigen binding domains are on same polypeptide chains.
119. The binding agent of any of the preceding embodiments, wherein each of the two antigen binding domains are on separate polypeptide chains.
120. The binding agent of any of the preceding embodiments, wherein the at least two antigen binding domains have the same specificity.
121. The binding agent of any of the preceding embodiments, wherein the at least two antigen binding domains have different specificities.
122. The binding agent of any of the preceding embodiments, wherein the binding agent comprises a Fc or Fc portion that is capable of binding to an Fc receptor of monocytes and/or macrophages.
123. The binding agent of any of the preceding embodiments, wherein the one or more antigen binding domains are in tandem.
124. The binding agent of any of the preceding embodiments, wherein the binding agent is naked.
125. The binding agent of any of the preceding embodiments, wherein the binding agent is conjugated to a therapeutic moiety.
126. The binding agent of any of the preceding embodiments, wherein the binding agent is conjugated to a detectable moiety.
127. The binding agent of any of the preceding embodiments, wherein the one or more antigen binding domains specifically bind to human TROP2.
128. The binding agent of any of the preceding embodiments, wherein the binding agent specifically binds human TROP2.
129. The binding agent of any of the preceding embodiments, wherein the binding agent is a single domain antibody comprising two identical heavy chain variable regions.
130. The binding agent of any of the preceding embodiments, wherein the binding agent is a single domain antibody comprising two identical heavy chains.
131. A composition comprising the binding agent or single domain antibody of any of the preceding embodiments.
132. A pharmaceutical composition comprising the binding agent or single domain antibody of any of the preceding embodiments and a pharmaceutically acceptable carrier.
133. A nucleic acid or set of nucleic acids encoding the binding agent or single domain antibody of any of the preceding embodiments.
134. A vector comprising the nucleic acid of any of the preceding embodiments.
135. A cell expressing the binding agent or single domain antibody of any of the preceding embodiments.
136. A cell comprising the nucleic acid or the vector of any of the preceding embodiments.
137. A kit comprising the binding agent or single domain antibody of any of the preceding embodiments.
138. A kit comprising the nucleic acid, vector, or cell of any of the preceding embodiments.
139. A method of treating a disorder or disease comprising administering the binding agent or single domain antibody of any one of the preceding embodiments or a pharmaceutical composition comprising the binding agent or single domain antibody of any of the preceding embodiments.
140. The method of any of the preceding embodiments, wherein the disorder or disease is associated with TROP2 expression or overexpression.
141. The method of any of the preceding embodiments, wherein the disorder or disease is cancer, solid tumor, epithetical cancer, lung cancer, metastatic lung cancer, small cell lung cancer, non-small cell lung cancer, myeloma, prostate cancer, breast cancer, triple negative breast cancer, rectal cancer, pancreatic cancer, glioblastoma, cervical cancer, colorectal cancer, gastric cancer, ovarian cancer, thyroid cancer, stomach cancer, urinary bladder cancer, cancer of the uterus, esophageal cancer, blood cancer, or head and neck cancer.

The embodiments and examples described herein are illustrative and are not meant to limit the scope of the claims. Variations of the foregoing embodiments, including alternatives, modifications and equivalents, are intended by the inventors to be encompassed by the claims.

Citations listed in the present application are incorporated herein by reference.

### REFERENCES

The entire content of all patents, patent applications and publications referred to throughout the application are incorporated herein by reference.
Angal, S. et al., A Single Amino Acid Substitution Abolishes the Heterogeneity of Chimeric Mouse/human IgG4 Antibody, Mol Immunol 30, 105-108, 1993
Cardillo et al., Clinical Cancer Research, Humanized Anti-Trop-2 IgG-SN-38 Conjugate for Effective Treatment of Diverse Epithelial Cancers: Preclinical Studies in Human Cancer Xenograft Models and Monkeys, May 2011 17:10
Chen X et al., Fusion Protein Linkers: Properties, Design and Functionality, Adv Drug Deliv Rev. 2013; 65(10):1357-1369.
De Vlieger et al., Single-domain Antibodies and their Formatting to Combat Viral Infections, Antibodies 8(1), 1-22, 2019
Deyev, S et al., Multivalency: the hallmark of antibodies used for optimization of tumor targeting by design, BioEssays, 30:904-918, 2008.
Godar M et al. Therapeutic Bispecific Antibody Formats: a Patent Applications Review (1994-2017) Expert Opinion on Therapeutic patents, 2018; 28(3):251-276
Goldenberg et al., The emergence of trophoblast cell-surface antigen 2 (TROP-2) as a novel cancer target, Oncotarget, 2018; 9:28989-29006
Goldenberg et al., Trop-2 is a novel target for solid cancer therapy with Sacituzumab govitecan (IMMU-132), antibody-drug conjugate (ADC), Oncotarget, 2015, 6:22496-22512
Ha, J-H et al. Immunoglobulin Fc Heterodimer Platform Technology: From Design to Applications in Therapeutic Antibodies and Proteins, Front Immunol, 2016; 7:394.
Hamers-Casterman C, et al., Naturally-occurring Antibodies Devoid of Light-chains. Nature 1993, 363:446-448.
Ikeda, M. et al., PrIE11, a novel anti-TROP2 antibody isolated by adenovirus-based antibody screening, recognizes a unique epitope, Biochemical and Biophysical Research Communications, 458 (2015) 877-882.
Kovalenko O V et al. Atypical Antigen Recognition Mode of a Shark Immunoglobulin New Antigen Receptor (IgNAR) Variable Domain Characterized by Humanization and Structural Analysis, J Biol Chem. 2013, 288:17408-17419
Muyldermans, S and Smider, 2016. Distinct Antibody Species: Structural Differences Creating Therapeutic Opportunities. Current Opinion in Immunology 2016, 40:7-13
Muyldermans, S et al., 1994. Sequence and Structure of VH Domain From Naturally Occurring Camel Heavy Chain Immunoglobulins Lacking Light Chains. Protein Eng. 7: 1129-1135.
Perrone et al., Frontiers in Oncology, Preclinical Activity of Sacituzumab Govitecan, an Antibody-Drug Conjugate Targeting Trophoblast Cell-Surface Antigen 2 (Trop-2) Linked to the Active Metabolite of Irinotecan (SN-38), in Ovarian Cancer, Feb. 12, 2020
Saunders K. O., Conceptual Approach to Modulating Antibody Effector Functions and Circulation Half-Life, Front. Immunol. 10:1296, 2019.
Stepan L. P., et al., Expression of Trop2 cell surface glycoprotein in normal and tumor tissues: Potential implications as a cancer therapeutic target, Journal of Histochemistry and Cytochemistry, 2011; 59:701-710
Tatusova, T. A, et al. (1999), BLAST 2 Sequences, a New Tool for Comparing Protein and Nucleotide Sequences, FEMS Microbiol Lett. 174:247-250)
Truong, A. et al., AR47A6.4.2, a naked monoclonal antibody targeting Trop-2, exhibits anti-tumor efficacy in multiple human cancer models as a monotherapeutic agent and demonstrates efficacy in combination therapy, Experimental and Molecular Therapeutics, AACR Annual Meeting, volume 68 (9), Abstract 3990, 2008.
Vincke C. et al. General Strategy to Humanize a Camelid Single-Domain Antibody and Identification of a Universal Humanized Nanobody Scaffold, J.Biol Chem. 2009, 284(5):3273-3284
Vu, K. B., et al., 1997. Comparison of Llama VH Sequences from Conventional and Heavy Chain Antibodies. Mol. Immunol. 34: 1121-1131.

### SEQUENCE TABLES

TABLE 15A
anti-TROP2 sequences

| Anti-TROP2 | CDR1 | CDR2 | CDR3 |
|---|---|---|---|
| KD001 | GSDMS | YITSGGTTYYPDSV | ARLTDSHYVEDA |
| Kabat CDRs | (SEQ ID NO: 1) | KG | (SEQ ID NO: 3) |
| | | (SEQ ID NO: 2) | |
| KD001 | GFTFSGSD | ITSGGTT | AKARLTDSHYVED |
| IMGT CDRs | (SEQ ID NO: 52) | (SEQ ID NO: 53) | A |
| | | | (SEQ ID NO: 54) |
| KD008 (KD001 | QVQLQESGGGMVHPGGSLRLSCAASGFTFSGSDMSWVRQAPGKG | | |
| variable region) | PEWVSYITSGGTTYYPDSVKGRFTISRDNAKNTLYLQMNSLTPDD | | |
| (SEQ ID NO: 22) | TALYRCAKARLTDSHYVEDAWGQGTQVTVSS | | |
| KD001 heavy | QVQLQESGGGMVHPGGSLRLSCAASGFTFSGSDMSWVRQAPGKG | | |
| chain | PEWVSYITSGGTTYYPDSVKGRFTISRDNAKNTLYLQMNSLTPDD | | |
| (SEQ ID NO: 29) | TALYRCAKARLTDSHYVEDAWGQGTQVTVSSEPKSCDKTHTCPP | | |
| | CPAPELLGGPSVFLFPPKPKDTLMISRTPEVTCVVVDVSHEDPEVK | | |
| | FNWYVDGVEVHNAKTKPREEQYNSTYRVVSVLTVLHQDWLNGK | | |
| | EYKCKVSNKALPAPIEKTISKAKGQPREPQVYTLPPSREEMTKNQ | | |
| | VSLTCLVKGFYPSDIAVEWESNGQPENNYKTTPPVLDSDGSFFLYS | | |
| | KLTVDKSRWQQGNVFSCSVMHEALHNHYTQKSLSLSPGK | | |
| KD002 | SADMS | YINADGSKTYYPDS | AKLTDTHYVEDY |
| Kabat CDRs | (SEQ ID NO: 4) | VKG | (SEQ ID NO: 6) |
| | | (SEQ ID NO: 5) | |
| KD002 | GFPFSSADMS | INADGSKT | ARAKLTDTHYVED |
| IMGT CDRs | (SEQ ID NO: 46) or | (SEQ ID NO: 47) | Y |
| | GFPFSSAD (SEQ ID | | (SEQ ID NO: 48) |
| | NO: 55) | | |
| KD009 (KD002 | QVQLQESGGGMVQVGGSLRLSCAASGFPFSSADMSWVRQAPGK | | |
| variable region) | GPEWVSYINADGSKTYYPDSVKGRFTISRDNAKNTLYLLMNNLKP | | |
| (SEQ ID NO: 23) | EDTALYRCARAKLTDTHYVEDYWGQGTQVTVSS | | |
| KD002 heavy | QVQLQESGGGMVQVGGSLRLSCAASGFPFSSADMSWVRQAPGK | | |
| chain | GPEWVSYINADGSKTYYPDSVKGRFTISRDNAKNTLYLLMNNLKP | | |
| (SEQ ID NO: 30) | EDTALYRCARAKLTDTHYVEDYWGQGTQVTVSSEPKSCDKTHTC | | |
| | PPCPAPELLGGPSVFLFPPKPKDTLMISRTPEVTCVVVDVSHEDPE | | |
| | VKFNWYVDGVEVHNAKTKPREEQYNSTYRVVSVLTVLHQDWLN | | |
| | GKEYKCKVSNKALPAPIEKTISKAKGQPREPQVYTLPPSREEMTK | | |
| | NQVSLTCLVKGFYPSDIAVEWESNGQPENNYKTTPPVLDSDGSFF | | |
| | LYSKLTVDKSRWQQGNVFSCSVMHEALHNHYTQKSLSLSPGK | | |
| KD003 | NYDMS | YINAGGSNTDYPDS | AKLTDTHYVEDY |
| Kabat CDRs | (SEQ ID NO: 7) | VKG | (SEQ ID NO: 9) |
| | | (SEQ ID NO: 8) | |
| KD003 | GFTFSNYD | INAGGSNT | ARAKLTDTHYVED |
| IMGT CDRs | (SEQ ID NO: 56) | (SEQ ID NO: 57) | Y |
| | | | (SEQ ID NO: 58) |
| KD010 (KD003 | QVQLQESGGGMVQPGGSLRLSCAASGFTFSNYDMSWVRQAPGK | | |
| variable region) | GPEWVSYINAGGSNTDYPDSVKGRFTISRDNAKNMLYLLMNNLK | | |
| (SEQ ID NO: 24) | PEDTALYRCARAKLTDTHYVEDYWGQGTQVTVSS | | |
| KD003 heavy | QVQLQESGGGMVQPGGSLRLSCAASGFTFSNYDMSWVRQAPGK | | |
| chain | GPEWVSYINAGGSNTDYPDSVKGRFTISRDNAKNMLYLLMNNLK | | |
| (SEQ ID NO: 31) | PEDTALYRCARAKLTDTHYVEDYWGQGTQVTVSSEPKSCDKTHT | | |
| | CPPCPAPELLGGPSVFLFPPKPKDTLMISRTPEVTCVVVDVSHEDPE | | |
| | VKFNWYVDGVEVHNAKTKPREEQYNSTYRVVSVLTVLHQDWLN | | |
| | GKEYKCKVSNKALPAPIEKTISKAKGQPREPQVYTLPPSREEMTK | | |
| | NQVSLTCLVKGFYPSDIAVEWESNGQPENNYKTTPPVLDSDGSFF | | |
| | LYSKLTVDKSRWQQGNVFSCSVMHEALHNHYTQKSLSLSPGK | | |
| KD004 | GSDMS | YITGSGTTYYPDSV | ARLTDSHYVEDA |
| Kabat CDRs | (SEQ ID NO: 10) | KG | (SEQ ID NO: 12) |
| | | (SEQ ID NO: 11) | |
| KD004 | GFTFSGSD | ITGSGTT | AKARLTDSHYVED |
| IMGT CDRs | (SEQ ID NO: 59) | (SEQ ID NO: 60) | A |
| | | | (SEQ ID NO: 61) |
| KD011 (KD004 | QVQLQESGGGLVQPGRSLRLSCAASGFTFSGSDMSWVRQAPGKG | | |
| variable region) | PEWVSYITGSGTTYYPDSVKGRFTTSRDNAKNMLYLQMNSLKPD | | |
| (SEQ ID NO: 25) | DTALYRCAKARLTDSHYVEDAWGQGTQVTVSS | | |
| KD004 heavy | QVQLQESGGGLVQPGRSLRLSCAASGFTFSGSDMSWVRQAPGKG | | |
| chain | PEWVSYITGSGTTYYPDSVKGRFTTSRDNAKNMLYLQMNSLKPD | | |
| (SEQ ID NO: 32) | DTALYRCAKARLTDSHYVEDAWGQGTQVTVSSEPKSCDKTHTCP | | |
| | PCPAPELLGGPSVFLFPPKPKDTLMISRTPEVTCVVVDVSHEDPEV | | |
| | KFNWYVDGVEVHNAKTKPREEQYNSTYRVVSVLTVLHQDWLNG | | |
| | KEYKCKVSNKALPAPIEKTISKAKGQPREPQVYTLPPSREEMTKN | | |
| | QVSLTCLVKGFYPSDIAVEWESNGQPENNYKTTPPVLDSDGSFFL | | |
| | YSKLTVDKSRWQQGNVFSCSVMHEALHNHYTQKSLSLSPGK | | |
| KD005 | GSDMS | YITSGGTTYYPDSV | SRLTDSHYVEDA |
| Kabat CDRs | (SEQ ID NO: 13) | KG | (SEQ ID NO: 15) |
| | | (SEQ ID NO: 14) | |
| KD005 | GFTFSGSDMS | ITSGGTT | AKSRLTDSHYVED |
| IMGT CDRs | (SEQ ID NO: 49) or | (SEQ ID NO: 50) | A |
| | GFTFSGSD (SEQ ID | | (SEQ ID NO: 51) |
| | NO: 62) | | |

| | |
|---|---|
| KD012 (KD005 | QVQLQESGGGLVEAGGSLRLSCAASGFTFSGSDMSWVRQAPGKG |
| variable region) | PEWVSYITSGGTTYYPDSVKGRFTISRDNAKNTLYLQMNSLKPDD |
| (SEQ ID NO: 26) | TALYRCAKSRLTDSHYVEDAWGQGTQVTVSS |
| KD005 heavy | QVQLQESGGGLVEAGGSLRLSCAASGFTFSGSDMSWVRQAPGKG |
| chain | PEWVSYITSGGTTYYPDSVKGRFTISRDNAKNTLYLQMNSLKPDD |
| (SEQ ID NO: 33) | TALYRCAKSRLTDSHYVEDAWGQGTQVTVSSEPKSCDKTHTCPP |
| | CPAPELLGGPSVFLFPPKPKDTLMISRTPEVTCVVVDVSHEDPEVK |
| | FNWYVDGVEVHNAKTKPREEQYNSTYRVVSVLTVLHQDWLNGK |
| | EYKCKVSNKALPAPIEKTISKAKGQPREPQVYTLPPSREEMTKNQ |
| | VSLTCLVKGFYPSDIAVEWESNGQPENNYKTTPPVLDSDGSFFLYS |
| | KLTVDKSRWQQGNVFSCSVMHEALHNHYTQKSLSLSPGK |
| KD015 heavy | QVQLQESGGGMVQVGGSLRLSCAASGFPFSSADMSWVRQAPGK |
| chain | GPEWVSYINADGSKTYYPDSVKGRFTISRDNAKNTLYLLMNNLKP |
| (SEQ ID NO: 44) | EDTALYRCARAKLTDTHYVEDYWGQGTQVTVSSESKYGPPCPPC |
| IgG4 version of | PAPEAAGGPSVFLFPPKPKDTLMISRTPEVTCVVVDVSQEDPEVQF |
| KD002 | NWYVDGVEVHNAKTKPREEQFNSTYRVVSVLTVLHQDWLNGKE |
| | YKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRL |
| | TVDKSRWQEGNVFSCSVMHEALHNHYTQKSLSLSLGK |
| KD016 heavy | QVQLQESGGGLVQPGRSLRLSCAASGFTFSGSDMSWVRQAPGKG |
| chain | PEWVSYITGSGTTYYPDSVKGRFTTSRDNAKNMLYLQMNSLKPD |
| (SEQ ID NO: 45) | DTALYRCAKARLTDSHYVEDAWGQGTQVTVSSESKYGPPCPPCP |
| IgG4 version of | APEAAGGPSVFLFPPKPKDTLMISRTPEVTCVVVDVSQEDPEVQFN |
| KD004 | WYVDGVEVHNAKTKPREEQFNSTYRVVSVLTVLHQDWLNGKEY |
| | KCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLT |
| | VDKSRWQEGNVFSCSVMHEALHNHYTQKSLSLSLGK |
| KD017 heavy | QVQLQESGGGMVQVGGSLRLSCAASGFPFSSADMSWVRQAPGK |
| chain | GPEWVSYINADGSKTYYPDSVKGRFTISRDNAKNTLYLLMNNLKP |
| (SEQ ID NO: | EDTALYRCARAKLTDTHYVEDYWGQGTQVTVSSESKYGPPCPPC |
| 177) | PAPEAAGGPSVFLFPPKPKDTLMISRTPEVTCVVVDVSQEDPEVQF |
| | NWYVDGVEVHNAKTKPREEQFNSTYRVVSVLTVLHQDWLNGKE |
| | YKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRL |
| | TVDKSRWQEGNVFSCSVMHEALHNHYTQKSLSLSLGK |
| KD018 heavy | QVQLQESGGGLVQPGRSLRLSCAASGFTFSGSDMSWVRQAPGKG |
| chain | PEWVSYITGSGTTYYPDSVKGRFTTSRDNAKNMLYLQMNSLKPD |
| (SEQ ID NO: | DTALYRCAKARLTDSHYVEDAWGQGTQVTVSSESKYGPPCPPCP |
| 178) | APEAAGGPSVFLFPPKPKDTLMISRTPEVTCVVVDVSQEDPEVQFN |
| | WYVDGVEVHNAKTKPREEQFNSTYRVVSVLTVLHQDWLNGKEY |
| | KCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLT |
| | VDKSRWQEGNVFSCSVMHEALHNHYTQKSLSLSLGK |
| KD019 heavy | QVQLQESGGGLVEAGGSLRLSCAASGFTFSGSDMSWVRQAPGKG |
| chain | PEWVSYITSGGTTYYPDSVKGRFTISRDNAKNTLYLQMNSLKPDD |
| (SEQ ID NO: 162 | TALYRCAKSRLTDSHYVEDAWGQGTQVTVSSESKYGPPCPPCPAP |
| | EAAGGPSVFLFPPKPKDTLMISRTPEVTCVVVDVSQEDPEVQFNW |
| | YVDGVEVHNAKTKPREEQFNSTYRVVSVLTVLHQDWLNGKEYK |
| | CKVSNKGLPSSIEKTISKAKGQPREPQVYTLPPSQEEMTKNQVSLT |
| | CLVKGFYPSDIAVEWESNGQPENNYKTTPPVLDSDGSFFLYSRLT |
| | VDKSRWQEGNVFSCSVMHEALHNHYTQKSLSLSLGK |

TABLE 15B
anti-TROP2 sequences - sharing CDRs with KD001

| Anti-TROP2 | CDR1 | CDR2 | CDR3 |
|---|---|---|---|
| KD071, KD072, | GSDMS | YITSGGTTYYPDSV | ARLTDSHYVEDA |
| KD073, KD074, | (SEQ ID NO: 215) | KG | (SEQ ID NO: 217) |
| KD087, KD088, | | (SEQ ID NO: 216) | |
| KD089, KD090, | | | |
| KD091, KD092, | | | |
| KD093, KD094, | | | |
| KD095, KD096 | | | |
| Kabat CDRs | | | |
| KD071, KD072, | GFTFSGSD | ITSGGTT | AKARLTDSHYVED |
| KD073, KD074, | (SEQ ID NO: 218) | (SEQ ID NO: 219) | A |
| KD087, KD088, | | | (SEQ ID NO: 220) |
| KD089, KD090, | | | |
| KD091, KD092, | | | |
| KD093, KD094, | | | |
| KD095, KD096 | | | |
| IMGT CDRs | | | |

| | |
|---|---|
| KD071 variable | QVQLQESGGGLVQAGGSLRLSCAASGFTFSGSDMSWVRQAPGKG |
| region | PEWVSYITSGGTTYYPDSVKGRFTISRDNAKNTLYLQMNSLKPDD |
| (SEQ ID NO: 221) | TALYRCAKARLTDSHYVEDAWGQGTQVTVSS |
| KD072 variable | QVQLQESGGGLVQPGGSLRLSCAASGFTFSGSDMSWVRQAPGKG |
| region | PEWVSYITSGGTTYYPDSVKGRFTISRDNAKNTLYLQMNSLKPDD |
| (SEQ ID NO: 222) | TALYRCAKARLTDSHYVEDAWGQGTQVTVSS |
| KD073 variable | QVQLQESGGGLVQPGGSLRLSCAASGFTFSGSDMSWVRQAPGKG |
| region | PEWVSYITSGGTTYYPDSVKGRFTISRDNAKNTLYLQMNSLTPDD |
| (SEQ ID NO: 223) | TALYRCAKARLTDSHYVEDAWGQGTQVTVSS |
| KD074 variable | QVQLQESGGGLVHPGGSLRLSCAASGFTFSGSDMSWVRQAPGKG |
| region | PEWVSYITSGGTTYYPDSVKGRFTISRDNAKNTLYLQMNSLKPDD |
| (SEQ ID NO: 224) | TALYRCAKARLTDSHYVEDAWGQGTQVTVSS |
| KD087 variable | QVQLQESGGGMVHPGGSLRLSCAASGFTFSGSDMSWVRQAPGKG |
| region | PEWVSYITSGGTTYYPDSVKGRFTISRDNAKNTLYLQMNSLTPDD |
| (SEQ ID NO: 362) | TALYRCAKARLTDSHYVEDAWGQGTQVTVSS |
| KD088 variable | QVQLQESGGGMVHPGGSLRLSCAASGFTFSGSDMSWVRQAPGKG |
| region | PEWVSYITSGGTTYYPDSVKGRFTISRDNAKNTLYLQMNSLKPDD |
| (SEQ ID NO: 363) | TALYRCAKARLTDSHYVEDAWGQGTQVTVSS |
| KD089 variable | QVQLQESGGGMVDPGGSLRLSCVASGFTFSGSDMSWVRQAPGKG |
| region | PEWVSYITSGGTTYYPDSVKGRFTISRDNAKNTLYLQMNSLKPDD |
| (SEQ ID NO: 364) | TALYRCAKARLTDSHYVEDAWGQGTQVTVSS |
| KD090 variable | QVQLQESGGGLVQPGGSLRLSCAASGFTFSGSDMSWVRQAPGKG |
| region | PEWVSYITSGGTTYYPDSVKGRFTISRDNANNMLYLQMNSLKPED |
| (SEQ ID NO: 365) | TALYRCAKARLTDSHYVEDAWGQGTQVTVSS |
| KD091 variable | QVQLQESGGGLVQPGDSLRLSCAASGFTFSGSDMSWVRQAPGKG |
| region | PEWVSYITSGGTTYYPDSVKGRFTISRDNAKNTLYLQMNSLKPDD |
| (SEQ ID NO: 366) | TALYRCAKARLTDSHYVEDAWGQGTQVTVSS |
| KD092 variable | QVQLQESGGGMVHPGGSLRLSCAASGFTFSGSDMSWVRQAPGKG |
| region | PEWVSYITSGGTTYYPDSVKGRFTISRDNAKNTLYLQMNSLKPDD |
| (SEQ ID NO: 367) | TALDRCAKARLTDSHYVEDAWGQGTQVTVSS |
| KD093 variable | QVQLQESGGGMVHPGGSLRLSCAASGFTFSGSDMSWVRQAPGKG |
| region | PEWVSYITSGGTTYYPDSVKGRFTISRDNANNMLYLQMNSLKPED |
| (SEQ ID NO: 368) | TALYRCAKARLTDSHYVEDAWGQGTQVTVSS |
| KD094 variable | QVQLQESGGGLVHPGGSLRLSCAASGFTFSGSDMSWVRQAPGKG |
| region | PEWVSYITSGGTTYYPDSVKGRFTISRDNAKNTLYLQMNSLKPDD |
| (SEQ ID NO: 369) | TALYRCAKARLTDSHYVEDAQGQGTQVTVSS |
| KD095 variable | QVQLQESGGGMVHPGGSLRLSCAASGFTFSGSDMSWVRQAPGKG |
| region | PEWVSYITSGGTTYYPDSVKGRFTISRDNAKNTLYLQMNSLKPDD |
| (SEQ ID NO: 370) | TALYRCAKARLTDSHYVEDAQGQGTQVTVSS |
| KD096 variable | QVQLQESGGGMVHPGGSLRLSCAASGFTFSGSDMSWVRQAPGKG |
| region | PEWVSYITSGGTTYYPDSVKGRFTISRDNAKNTLYLQMNSLTPDD |
| (SEQ ID NO: 371) | TALYRCAKARLTDSHYVEDAQGQGTQVTVSS |

XVQLXESGGGXXXXGXSLRLSCXASGFTFSGSDMSWVRQAPGKGXEWVSYITSGGT
TYYXDXVKGRFTISRDNXXNXLYLQMNSLXXXDTAXXRCAKARLTDSHYVEDAXG
QGTXVTVSS (SEQ ID NOs: 507-511)

TABLE 15C
Exemplary humanized version of KD008

| | SEQUENCE |
|---|---|
| SEQ ID NO: | IMGT CDRs |
| KD132 variable | EVQLLESGGGMVQPGGSLRLSCAASGFTFSGSDMSWVRQAPGKG |
| region | LEWVSYITSGGTTYYADTVKGRFTISRDNSKNTLYLQMNSLRAED |
| SEQ ID NO: 469 | TAVYRCAKARLTDSHYVEDAWGQGTLVTVSS |
| KD133 variable | EVQLLESGGGLVQPGGSLRLSCAASGFTFSGSDMSWVRQAPGKG |
| region | LEWVSYITSGGTTYYADTVKGRFTISRDNSKNTLYLQMNSLRAED |
| SEQ ID NO: 470 | TAVYRCAKARLTDSHYVEDAWGQGTLVTVSS |
| KD133 | EVQLLESGGGLVQPGGSLRLSCAASGFTFSGSDMSWVRQAPGKG |
| SEQ ID NO: 482 | LEWVSYITSGGTTYYADTVKGRFTISRDNSKNTLYLQMNSLRAED |
| | TAVYRCAKARLTDSHYVEDAWGQGTLVTVSSEPKSCDKTHTCPP |
| | CPAPELLGGPSVFLFPPKPKDTLMISRTPEVTCVVVDVSHEDPEVK |
| | FNWYVDGVEVHNAKTKPREEQYNSTYRVVSVLTVLHQDWLNGK |
| | EYKCKVSNKALPAPIEKTISKAKGQPREPQVYTLPPSREEMTKNQ |
| | VSLTCLVKGFYPSDIAVEWESNGQPENNYKTTPPVLDSDGSFFLYS |
| | KLTVDKSRWQQGNVFSCSVMHEALHNHYTQKSLSLSPGK |
| KD134 variable | QVQLQESGGGMIQPGGSLRLSCAASGFTFSGSDMSWVRQAPGKG |
| region | PEWVSYITSGGTTYYPDTVKGRFTISRDNSKNTLYLQMNSLRADD |
| SEQ ID NO: 471 | TALYRCAKARLTDSHYVEDAWGQGTLVTVSS |
| KD135 variable | QVQLQESGGGLIQPGGSLRLSCAASGFTFSGSDMSWVRQAPGKGP |
| region | EWVSYITSGGTTYYPDTVKGRFTISRDNSKNTLYLQMNSLRADDT |
| SEQ ID NO: 472 | ALYRCAKARLTDSHYVEDAWGQGTLVTVSS |
| KD135 | QVQLQESGGGLIQPGGSLRLSCAASGFTFSGSDMSWVRQAPGKGP |
| SEQ ID NO: 483 | EWVSYITSGGTTYYPDTVKGRFTISRDNSKNTLYLQMNSLRADDT |
| | ALYRCAKARLTDSHYVEDAWGQGTLVTVSSEPKSCDKTHTCPPC |
| | PAPELLGGPSVFLFPPKPKDTLMISRTPEVTCVVVDVSHEDPEVKF |
| | NWYVDGVEVHNAKTKPREEQYNSTYRVVSVLTVLHQDWLNGKE |
| | YKCKVSNKALPAPIEKTISKAKGQPREPQVYTLPPSREEMTKNQVS |
| | LTCLVKGFYPSDIAVEWESNGQPENNYKTTPPVLDSDGSFFLYSKL |
| | TVDKSRWQQGNVFSCSVMHEALHNHYTQKSLSLSPGK |
| KD136 VHH | QVQLQESGGGLVHPGGSLRLSCAASGFTFSGSDMSWVRQAPGKG |
| SEQ ID NO: 473 | PEWVSYITSGGTTYYPDTVKGRFTISRDNAKNTLYLQMNSLTPED |
| | TALYRCAKARLTDSHYVEDAWGQGTQVTVSS |
| KD136 | QVQLQESGGGLVHPGGSLRLSCAASGFTFSGSDMSWVRQAPGKG |
| SEQ ID NO: 484 | PEWVSYITSGGTTYYPDTVKGRFTISRDNAKNTLYLQMNSLTPED |
| | TALYRCAKARLTDSHYVEDAWGQGTQVTVSSEPKSCDKTHTCPP |
| | CPAPELLGGPSVFLFPPKPKDTLMISRTPEVTCVVVDVSHEDPEVK |
| | FNWYVDGVEVHNAKTKPREEQYNSTYRVVSVLTVLHQDWLNGK |
| | EYKCKVSNKALPAPIEKTISKAKGQPREPQVYTLPPSREEMTKNQ |
| | VSLTCLVKGFYPSDIAVEWESNGQPENNYKTTPPVLDSDGSFFLYS |
| | KLTVDKSRWQQGNVFSCSVMHEALHNHYTQKSLSLSPGK |

XVQLXESGGGXXXXGXSLRLSCXASGFTFSGSDMSWVRQAPGKGXEWVSYITSGGT
TYYXDXVKGRFTISRDNXXNXLYLQMNSLXXXDTAXXRCAKARLTDSHYVEDAXG
QGTXVTVSS (SEQ ID NOs: 507-511)

TABLE 15D
anti-TROP2 sequences sharing CDRs with KD011

| Anti-TROP2 | CDR1 | CDR2 | CDR3 |
|---|---|---|---|
| KD080 | GSDMS | YITGSGTTYYPDSV | ARLTDSHYVEDA |
| Kabat CDRs | (SEQ ID NO: 298) | KG | (SEQ ID NO: 300) |
| | | (SEQ ID NO: 299) | |
| KD080 | GFTFSGSD | ITGSGTT | AKARLTDSHYVED |
| IMGT CDRs | (SEQ ID NO: 301) | (SEQ ID NO: 302) | A |
| | | | (SEQ ID NO: 303) |

| | |
|---|---|
| KD080 variable | QVQLQESGGGLVQPGGSLRLSCAASGFTFSGSDMSWVRQAPGKG |
| region | PEWVSYITGSGTTYYPDSVKGRFTISRDNAKNTLYLQMNSLKPDD |
| (SEQ ID NO: 304) | TALYRCAKARLTDSHYVEDAWGQGTQVTVSS |

QVQLQESGGGLVQPGXSLRLSCAASGFTFSGSDMSWVRQAPGKGPEWVSYITGSGTT
YYPDSVKGRFTXSRDNAKNXLYLQMNSLKPDDTALYRCAKARLTDSHYVEDAWGQ
GTQVTVSS (SEQ ID NO: 512-513)

TABLE 15E
anti-TROP2 sequences sharing CDRs

| Anti-TROP2 | CDR1 | CDR2 | CDR3 |
|---|---|---|---|
| KD075, KD076, | GSDMS | YITGGGTTYYPDSV | ARLTDSHYVEDA |
| KD097, KD098, | (SEQ ID NO: 225) | KG | (SEQ ID NO: 227) |
| KD099, KD100, | | (SEQ ID NO: 226) | |
| KD101, KD102, | | | |
| KD103, KD104, | | | |
| KD105, KD106, | | | |
| KD107, KD108, | | | |
| KD109, KD110 | | | |
| Kabat CDRs | | | |
| KD075, KD076, | GFTFSGSD | ITGGGTT | AKARLTDSHYVED |
| KD097, KD098, | (SEQ ID NO: 228) | (SEQ ID NO: 229) | A |
| KD099, KD100, | | | (SEQ ID NO: 230) |
| KD101, KD102, | | | |
| KD103, KD104, | | | |
| KD105, KD106, | | | |
| KD107, KD108, | | | |
| KD109, KD110 | | | |
| IMGT CDRs | | | |

| | |
|---|---|
| KD075 variable | QVQLQESGGGLVQAGGSLRLSCAASGFTFSGSDMSWVRQAPGKG |
| region | PEWVSYITGGGTTYYPDSVKGRFTISRDNAKNTLYLQMNSLKPDD |
| (SEQ ID NO: 231) | TALYRCAKARLTDSHYVEDAWGQGTQVTVSS |
| KD076 | QVQLQESGGGLVQPGGSLRLSCAASGFTFSGSDMSWVRQAPGKG |
| variable region | PEWVSYITGGGTTYYPDSVKGRFTISRDNAKNTLYLQMNSLKPDD |
| (SEQ ID NO: 232) | TALYRCAKARLTDSHYVEDAWGQGTQVTVSS |
| KD097 | QVQLQESGGGLVQPGGSLRLSCAASGFTFSGSDMSWVRQAPGKG |
| variable region | PEWVSYITGGGTTYYPDSVKGRFTISRDNANNMLYLQMNSLKPE |
| (SEQ ID NO: 295) | DTALYRCAKARLTDSHYVEDAWGQGTQVTVSS |
| KD098 | QVQLQESGGGMVQPGGSLRLSCAASGFTFSGSDMSWVRQAPGKG |
| variable region | PEWVSYITGGGTTYYPDSVKGRFTISRDNANNMLYLQMNSLKPE |
| (SEQ ID NO: 296) | DTALYRCAKARLTDSHYVEDAWGQGTQVTVSS |
| KD099 | QVQLQESGGGMVHPGGSLRLSCAASGFTFSGSDMSWVRQAPGKG |
| variable region | PEWVSYITGGGTTYYPDSVKGRFTISRDNANNMLYLQMNSRKPE |
| (SEQ ID NO: 297) | DTAVYRCAKARLTDSHYVEDAWGQGTQVTVSS |
| KD100 | QVQLQESGGGMVHPGGSLRLSCVASGFTFSGSDMSWVRQAPGKG |
| variable region | PEWVSYITGGGTTYYPDSVKGRFTISRDNAKNTLYLQMNSLKPDD |
| (SEQ ID NO: 372) | TALYRCAKARLTDSHYVEDAWGQGTQVTVSS |
| KD101 | QVQLQESGGGMVHPGGSLRLSCAASGFTFSGSDMSWVRQAPGKG |
| variable region | PEWVSYITGGGTTYYPDSVKGRFTISRDNAKNTLYLQMNSLKPDD |
| (SEQ ID NO: 373) | TALYRCAKARLTDSHYVEDAWGQGTQVTVSS |
| KD102 | QVQLQESGGGMVHPGGSLRLSCAASGFTFSGSDMSWVRQAPGKG |
| variable region | PEWVSYITGGGTTYYPDSVKGRFTISRDNANNMLYLQMNSLKPE |
| (SEQ ID NO: 374) | DTALYRCAKARLTDSHYVEDAWGQGTQVTVSS |
| KD103 | QVQLQESGGGMVHPGGSLRLSCAASGFTFSGSDMSWVRQAPGKG |
| variable region | PEWGSYITGGGTTYYPDSVKGRFTISRDNAKNTLYLQMNSLKPDD |
| (SEQ ID NO: 375) | TALYRCAKARLTDSHYVEDAWGQGTQVTVSS |
| KD104 | QVQLQESGGGLVHPGGSLRLSCAASGFTFSGSDMSWVRQAPGKG |
| variable region | PEWVSYITGGGTTYYPDSVKGRFTISRDNAKNTLYLQMNSLKPDD |
| (SEQ ID NO: 376) | TALYRCAKARLTDSHYVEDAWGQGTQVTVSS |
| KD105 | QVQLQESGGGLVQAGGSLRLSCVASGFTFSGSDMSWVRQAPGKG |
| variable region | PEWVSYITGGGTTYYPDSVKGRFTISRDNAKNTLYLQMNSLKPDD |
| (SEQ ID NO: 377) | TALYRCAKARLTDSHYVEDAWGQGTQVTVSS |
| KD106 | QVQLQESGGGLVQAGGSLRLSCAASGFTFSGSDMSWVRQAPGKG |
| variable region | PEWVSYITGGGTTYYPDSVKGRFTISRDNANNMLYLQMNSLKPE |
| (SEQ ID NO: 378) | DTALYRCAKARLTDSHYVEDAWGQGTQVTVSS |
| KD107 | QVQLQESGGGLVQPGGSLRLSCVASGFTFSGSDMSWVRQAPGKG |
| variable region | PEWVSYITGGGTTYYPDSVKGRFTISRDNAKNTLYLQMNSLKPDD |
| (SEQ ID NO: 379) | TALYRCAKARLTDSHYVEDAWGQGTQVTVSS |
| KD108 | QVQLQESGGGMVHPGGSLRLSCAASGFTFSGSDMSWVRQAPGKG |
| variable region | PEWVSYITGGGTTYYPDSVKGRFTISRDNAKNTLYLQMNSLKPDD |
| (SEQ ID NO: 380) | TALYRCAKARLTDSHYVEDAQGQGTQVTVSS |
| KD109 | QVQLQESGGGMVQPGGSLRLSCAASGFTFSGSDMSWVRQAPGKG |
| variable region | PEWVSYITGGGTTYYPDSVKGRFTISRDNAKNTLYLQMNSLKPDD |
| (SEQ ID NO: 381) | TALYRCAKARLTDSHYVEDAQGQGTQVTVSS |
| KD110 | QVQLQESGGGMVHPGGSLRLSCVASGFTFSGSDMSWVRQAPGKG |
| variable region | PEWVSYITGGGTTYYPDSVKGRFTISRDNAKNTLYLQMNSLKPDD |
| (SEQ ID NO: 382) | TALYRCAKARLTDSHYVEDAQGQGTQVTVSS |

QVQLQESGGGXVXXGGSLRLSCXASGFTFSGSDMSWVRQAPGKGPEWXSYITGGGT
TYYPDSVKGRFTISRDNAXNXLYLQMNSXKPXDTAXYRCAKARLTDSHYVEDAXGQ
GTQVTVSS (SEEQ ID NOs: 514-516)

TABLE 15F
anti-TROP2 sequences sharing CDRs

| Anti-TROP2 | CDR1 | CDR2 | CDR3 |
|---|---|---|---|
| KD077, KD078, | GSDMS | YITAGGTTYYPDSV | ARLTDSHYVEDA |
| KD111, KD112, | (SEQ ID NO: 233) | KG | (SEQ ID NO: 235) |
| KD113, KD114, | | (SEQ ID NO: 234) | |
| KD115 | | | |
| Kabat CDRs | | | |
| KD077, KD078, | GFTFSGSD | ITAGGTT | AKARLTDSHYVED |
| KD111, KD112, | (SEQ ID NO: 236) | (SEQ ID NO: 237) | A |
| KD113, KD114, | | | (SEQ ID NO: 238) |
| KD115 | | | |
| IMGT CDRs | | | |

| | |
|---|---|
| KD077 | QVQLQESGGGLVQAGGSLRLSCAASGFTFSGSDMSWVRQAPGKG |
| variable region | PEWVSYITAGGTTYYPDSVKGRFTISRDNAKNTLYLQMNSLKPDD |
| (SEQ ID NO: 239) | TALYRCAKARLTDSHYVEDAWGQGTQVTVSS |
| KD078 | QVQLQESGGGLVQPGGSLRLSCAASGFTFSGSDMSWVRQAPGKG |
| variable region | PEWVSYITAGGTTYYPDSVKGRFTISRDNAKNTLYLQMNSLKPDD |
| (SEQ ID NO: 240) | TALYRCAKARLTDSHYVEDAWGQGTQVTVSS |
| KD111 variable | QVQLQESGGGMVHPGGSLRLSCAASGFTFSGSDMSWVRQAPGKG |
| region | PEWVSYITAGGTTYYPDSVKGRFTISRDNAKNTLYLQMNSLKPDD |
| (SEQ ID NO: 383) | TALYRCAKARLTDSHYVEDAQGQGTQVTVSS |
| KD112 variable | QVQLQESGGGLVQPGGSLRLSCAASGFTFSGSDMSWVRQAPGKG |
| region | PEWVSYITAGGTTYYPDSVKGRFTISRDNAKNTLYLQMNSLKPDD |
| (SEQ ID NO: 384) | TALYRCAKARLTDSHYVEDAQGQGTQVTVSS |
| KD113 variable | QVQLQESGGGLVQPGGSLRLSCVASGFTFSGSDMSWVRQAPGKG |
| region | PEWVSYITAGGTTYYPDSVKGRFTISRDNAKNTLYLQMNSLKPDD |
| (SEQ ID NO: 385) | TALYRCAKARLTDSHYVEDAWGQGTQVTVSS |
| KD114 variable | QVQLQESGGGLVHPGGSLRLSCAASGFTFSGSDMSWVRQAPGKG |
| region | PEWVSYITAGGTTYYPDSVKGRFTISRDNAKNTLYLQMNSLKPDD |
| (SEQ ID NO: 386) | TALYRCAKARLTDSHYVEDAWGQGTQVTVSS |
| KD115 variable | QVQLQESGGGMVHPGGSLRLSCAASGFTFSGSDMSWVRQAPGKG |
| region | PEWVSYITAGGTTYYPDSVKGRFTISRDNAKNTLYLQMNSLKPDD |
| (SEQ ID NO: 387) | TALYRCAKARLTDSHYVEDAWGQGTQVTVSS |

QVQLQESGGGXVXXGGSLRLSCXASGFTFSGSDMSWVRQAPGKGPEWVSYITAGGT
TYYPDSVKGRFTISRDNAKNTLYLQMNSLKPDDTALYRCAKARLTDSHYVEDAXGQ
GTQVTVSS (SEQ ID NOs: 517-519)

TABLE 15G
anti-TROP2 sequences sharing CDRs

| Anti-TROP2 | CDR1 | CDR2 | CDR3 |
|---|---|---|---|
| KD079, KD116 | GSDMS | YITSGGGTYYPDSV | ARLTDSHYVEDA |
| Kabat CDRs | (SEQ ID NO: 241) | KG | (SEQ ID NO: 243) |
| | | (SEQ ID NO: 242) | |
| KD079, KD116 | GFTFSGSD | ITSGGGT | AKARLTDSHYVED |
| IMGT CDRs | (SEQ ID NO: 244) | (SEQ ID NO: 245) | A |
| | | | (SEQ ID NO: 246) |

| | |
|---|---|
| KD079 variable | QVQLQESGGGLVQPGGSLRLSCAASGFTFSGSDMSWVRQAPGKG |
| region | PEWVSYITSGGGTYYPDSVKGRFTISRDNAKNTLYLQMNSLKPED |
| (SEQ ID NO: 247) | TALYRCAKARLTDSHYVEDAWGQGTQVTVSS |
| KD116 variable | QVQLQESGGGMVHPGGSLRLSCAASGFTFSGSDMSWVRQAPGKG |
| region | PEWVSYITSGGGTYYPDSVKGRFTISRDNAKNTLYLQMNSLKPED |
| (SEQ ID NO: 388) | TALYRCAKARLTDSHYVEDAWGQGTQVTVSS |

QVQLQESGGGXVXPGGSLRLSCAASGFTFSGSDMSWVRQAPGKGPEWVSYITSGGGT
YYPDSVKGRFTISRDNAKNTLYLQMNSLKPEDTALYRCAKARLTDSHYVEDAWGQG
TQVTVSS (SEQ ID NOs: 520-522)

TABLE 15H
anti-TROP2 sequences sharing CDRs

| Anti-TROP2 | CDR1 | CDR2 | CDR3 |
|---|---|---|---|
| KD082, KD118 | GSDMS | YITGGGGTYYADSV | ARLTDSHYVEDA |
| Kabat CDRs | (SEQ ID NO: 319) | KG | (SEQ ID NO: 321) |
| | | (SEQ ID NO: 320) | |
| KD082, KD118 | GFTFSGSD | ITGGGGT | AKARLTDSHYVED |
| IMGT CDRs | (SEQ ID NO: 322) | (SEQ ID NO: 323) | A |
| | | | (SEQ ID NO: 324) |

| | |
|---|---|
| KD082 variable | QVQLQESGGGMVHPGGSLRLSCAASGFTFSGSDMSWVRQAPGKG |
| region | PEWVSYITGGGGTYYADSVKGRFTISRDHAKNMLYLQMNSLKPE |
| (SEQ ID NO: 325) | DTALYRCAKARLTDSHYVEDAWGQGTQVTVSS |
| KD118 variable | QVQLQESGGGMVHPGGSLRLSCAASGFTFSGSDMSWVRQAPGKG |
| region | PEWVSYITGGGGTYYADSVKGRFTISRDNAKNTLYLQMNSLKPE |
| (SEQ ID NO: 390) | DTALYRCAKARLTDSHYVEDAWGQGTQVTVSS |

QVQLQESGGGMVHPGGSLRLSCAASGFTFSGSDMSWVRQAPGKGPEWVSYITGGGG
TYYADSVKGRFTISRDXAKNXLYLQMNSLKPEDTALYRCAKARLTDSHYVEDAWGQ
GTQVTVSS (SEQ ID NOS:523-525)

TABLE 15I
anti-TROP2 sequences sharing CDRs

| Anti-TROP2 | CDR1 | CDR2 | CDR3 |
|---|---|---|---|
| KD119, KD120 | GSDMS | YITAGGSTEYADSV | ARLTDSHYVEDA |
| Kabat CDRs | (SEQ ID NO: 391) | KG | (SEQ ID NO: 393) |
| | | (SEQ ID NO: 392) | |
| KD119, KD120 | GFTFSGSD | ITAGGST | AKARLTDSHYVED |
| IMGT CDRs | (SEQ ID NO: 394) | (SEQ ID NO: 395) | A |
| | | | (SEQ ID NO: 396) |

| | |
|---|---|
| KD119 variable | QVQLQESGGGMVLPGGSLRLSCAASGFTFSGSDMSWVRQAPGKG |
| region | PEWVSYITAGGSTEYADSVKGRFTISRDNAKKKLYLQMNSLKPED |
| (SEQ ID NO: 397) | TALYRCAKARLTDSHYVEDAWGQGTQVTVSS |
| KD120 variable | QVQLQESGGGLVQPGGSLRLSCAASGFTFSGSDMSWVRQAPGKG |
| region | PEWVSYITAGGSTEYADSVKGRFTISRDNAKKKLYLQMNSLKPED |
| (SEQ ID NO: 398) | TALYRCAKARLTDSHYVEDAWGQGTQVTVSS |

QVQLQESGGGXVXPGGSLRLSCAASGFTFSGSDMSWVRQAPGKGPEWVSYITAGGST
EYADSVKGRFTISRDNAKKKLYLQMNSLKPEDTALYRCAKARLTDSHYVEDAWGQG
TQVTVSS (SEQ ID NOs: 526-528)

TABLE 15J
anti-TROP2 sequences sharing CDRs

| Anti-TROP2 | CDR1 | CDR2 | CDR3 |
|---|---|---|---|
| KD081, KD117 | SYDMS | TINTGGGSTYYADS | ARLTDSHYVEDA |
| Kabat CDRs | (SEQ ID NO: 305) | VKG | (SEQ ID NO: 307) |
| | | (SEQ ID NO: 306) | |
| KD081, KD117 | GFTFSSYD | INTGGGST | AKARLTDSHYVED |
| IMGT CDRs | (SEQ ID NO: 308) | (SEQ ID NO: 309) | A |
| | | | (SEQ ID NO: 310) |

| | |
|---|---|
| KD081 variable | QVQLQESGGGMVQPGGSLRLSCAASGFTFSSYDMSWVRQAPGKG |
| region | PEWVSTINTGGGSTYYADSVKGRFTISRDNANNMLYLQMNSLKP |
| (SEQ ID NO: 311) | EDTALYRCAKARLTDSHYVEDAWGQGTQVTVSS |
| KD117 variable | QVQLQESGGGLVQPGGSLRLSCAASGFTFSSYDMSWVRQAPGKG |
| region | PEWVSTINTGGGSTYYADSVKGRFTISRDNAKNTLYLQMNSLKPD |
| (SEQ ID NO: 389) | DTALYRCAKARLTDSHYVEDAWGQGTQVTVSS |

QVQLQESGGGXVQPGGSLRLSCAASGFTFSSYDMSWVRQAPGKGPEWVSTINTGGGS
TYYADSVKGRFTISRDNAXNXLYLQMNSLKPXDTALYRCAKARLTDSHYVEDAWGQ
GTQVTVSS (SEQ ID NOs: 529-531)

TABLE 15K
anti-TROP2 sequences unique CDR combinations

| Anti-TROP2 | CDR1 | CDR2 | CDR3 |
|---|---|---|---|
| KD131 | SYDMS | TINTDGGSTYYADS | ARLTDSHYVEDA |
| Kabat CDRs | (SEQ ID NO: 312) | VKD | (SEQ ID NO: 314) |
| | | (SEQ ID NO: 313) | |
| KD131 | GFTFSSYD | INTDGGST | AKARLTDSHYVED |
| IMGT CDRs | (SEQ ID NO: 315) | (SEQ ID NO: 316) | A |
| | | | (SEQ ID NO: 317) |
| KD131 variable | QVQLQESGGGMVQPGGSLRLSCAASGFTFSSYDMSWVRQAPGKG | | |
| region | PEWLSTINTDGGSTYYADSVKDRFTISRDNAKNTLYLQMNSLTPD | | |
| (SEQ ID NO: 318) | DTALYRCAKARLTDSHYVEDAWGQGTQVTVSS | | |
| KD083 | DSDMS | YITGGGGTYYADSV | ARLTDSHYVEDA |
| Kabat CDRs | (SEQ ID NO: 326) | KG | (SEQ ID NO: 328) |
| | | (SEQ ID NO: 327) | |
| KD083 | GFTFSDSD | ITGGGGT | AKARLTDSHYVED |
| IMGT CDRs | (SEQ ID NO: 329) | (SEQ ID NO: 330) | A |
| | | | (SEQ ID NO: 331) |
| KD083 variable | QVQLQESGGGLVQPGGSLRLSCAASGFTFSDSDMSCVRQAPGTGP | | |
| region | EWVSYITGGGGTYYADSVKGRFTISRDNAKNMLYLQMNSLKPED | | |
| (SEQ ID NO: 332) | TALYRCAKARLTDSHYVEDAWGQGTQVTVSS | | |
| KD084 | SADMS | YINADGSKTYYPDS | ARLTDSHYVEDA |
| Kabat CDRs | (SEQ ID NO: 333) | VKG | (SEQ ID NO: 335) |
| | | (SEQ ID NO: 334) | |
| KD084 | GFPFSSAD | INADGSKT | AKARLTDSHYVED |
| IMGT CDRs | (SEQ ID NO: 336) | (SEQ ID NO: 337) | A |
| | | | (SEQ ID NO: 338) |
| KD084 variable | QVQLQESGGGMVQVGGSLRLSCAASGFPFSSADMSWVRQAPGK | | |
| region | GPEWVSYINADGSKTYYPDSVKGRFTISRDNAKNTLYLLMNNLRP | | |
| (SEQ ID NO: 339) | EDTALYRCAKARLTDSHYVEDAWGQGTQVTVSS | | |
| KD085 | GSDMS | SLTSGGTTYYPDSV | ARLTDSHYVEDA |
| Kabat CDRs | (SEQ ID NO: 340) | KG | (SEQ ID NO: 342) |
| | | (SEQ ID NO: 341) | |
| KD085 | GFTFSGSD | LTSGGTT | AKARLTDSHYVED |
| IMGT CDRs | (SEQ ID NO: 343) | (SEQ ID NO: 344) | A |
| | | | (SEQ ID NO: 345) |
| KD085 variable | QVQLQESGGGMVHPGGSLRLSCAASGFTFSGSDMSWVRQVPGQG | | |
| region | PEWVSSLTSGGTTYYPDSVKGRFSISRDNAKNTLYLQMNSLKPDD | | |
| (SEQ ID NO: 346) | TALYRCAKARLTDSHYVEDAWGQGTHVTVSS | | |
| KD086 | NYAMS | YITSGGGTYYPDSV | ARLTDSHYVEDA |
| Kabat CDRs | (SEQ ID NO: 347) | KG | (SEQ ID NO: 349) |
| | | (SEQ ID NO: 348) | |
| KD086 | GFSFNNYA | ITSGGGT | AKARLTDSHYVED |
| IMGT CDRs | (SEQ ID NO: 350) | (SEQ ID NO: 351) | A |
| | | | (SEQ ID NO: 352) |
| KD086 variable | QVQLQESGGGLVQPGGSLRLSCAASGFSFNNYAMSWVRQAPGKG | | |
| region | PEWVSYITSGGGTYYPDSVKGRFTISRDNAKNTLYLQMNSLKPED | | |
| (SEQ ID NO: 353) | TALYRCAKARLTDSHYVEDAWGQGTQVTVSS | | |
| KD121 | GSDMS | YITGGGTTYYPDSV | ARLTDSHYVEDA |
| Kabat CDRs | (SEQ ID NO: 399) | KG | (SEQ ID NO: 401) |
| | | (SEQ ID NO: 400) | |
| KD121 IMGT | GFAFSGSD | ITGGGTT | AKARLTDSHYVED |
| CDRs | (SEQ ID NO: 402) | (SEQ ID NO: 403) | A |
| | | | (SEQ ID NO: 404) |
| KD121 variable | QVQLQESGGGMVHPGGSLRLSCAASGFAFSGSDMSWVRQAPGK | | |
| region | GPEWVSYITGGGTTYYPDSVKGRFTISRDNAKNTLYLQMNSLKPD | | |
| (SEQ ID NO: 405) | DTALYRCAKARLTDSHYVEDAWGQGTQVTVSS | | |
| KD122 | GSDMS | YITGGGSTYYADSV | ARLTDSHYVEDA |
| Kabat CDRs | (SEQ ID NO: 406) | KG | (SEQ ID NO: 408) |
| | | (SEQ ID NO: 407) | |
| KD122 IMGT | GFTFSGSD | ITGGGST | AKARLTDSHYVED |
| CDRs | (SEQ ID NO: 409) | (SEQ ID NO: 410) | A |
| | | | (SEQ ID NO: 411) |
| KD122 variable | QVQLQESGGGMVHPGGSLRLSCASSGFTFSGSDMSWVRQAPGKG | | |
| region | PEWVSYITGGGSTYYADSVKGRFTISRDNAKNTLYLQMNNLKPE | | |
| (SEQ ID NO: 412) | DTALYRCAKARLTDSHYVEDAWGQGTQVTVSS | | |
| KD123 | GSDMS | YITAGGTTYYPDSV | ARLTDSHYVEDA |
| Kabat CDRs | (SEQ ID NO: 413) | KG | (SEQ ID NO: 415) |
| | | (SEQ ID NO: 414) | |
| KD123 | GRTFSGSD | ITAGGTT | AKARLTDSHYVED |
| IMGT CDRs | (SEQ ID NO: 416) | (SEQ ID NO: 417) | A |
| | | | (SEQ ID NO: 418) |
| KD123 variable | QVQLQESGGGLVQAGGSLRLSCAASGRTFSGSDMSWVRQAPGKG | | |
| region | PEWVSYITAGGTTYYPDSVKGRFTISRDNAKNTLYLQMNSLKPDD | | |
| (SEQ ID NO: 419) | TALYRCAKARLTDSHYVEDAWGQGTQVTVSS | | |
| KD124 | GSDMS | YITSGGTTYYPDSV | VRLTDSHYVEDA |
| Kabat CDRs | (SEQ ID NO: 420) | KG | (SEQ ID NO: 422) |
| | | (SEQ ID NO: 421) | |
| KD124 | GFTFSGSD | ITSGGTT | AKVRLTDSHYVED |
| IMGT CDRs | (SEQ ID NO: 423) | (SEQ ID NO: 424) | A |
| | | | (SEQ ID NO: 425) |
| KD124 variable | QVQLQESGGGMVHPGGSLRLSCAASGFTFSGSDMSWVRQAPGKG | | |
| region | PEWVSYITSGGTTYYPDSVKGRFTISRDNAKNTLYLQMNSLKPDD | | |
| (SEQ ID NO: 426) | TALYRCAKVRLTDSHYVEDAWGQGTQVTVSS | | |
| KD125 | GSDMS | YITGGGTTYYPDSV | ARLTDGHYVEDA |
| Kabat CDRs | (SEQ ID NO: 427) | KG | (SEQ ID NO: 429) |
| | | (SEQ ID NO: 428) | |
| KD125 | GFTFSGSD | ITGGGTT | AKARLTDGHYVED |
| IMGT CDRs | (SEQ ID NO: 430) | (SEQ ID NO: 431) | A |
| | | | (SEQ ID NO: 432) |
| KD125 variable | QVQLQESGGGMVHPGGSLRLSCAASGFTFSGSDMSWVRQAPGKG | | |
| region | PEWVSYITGGGTTYYPDSVKGRFTISRDNAKNTLYLQMNSLKPDD | | |
| (SEQ ID NO: 433) | TALYRCAKARLTDGHYVEDAWGQGTQVTVSS | | |
| KD126 | GSDMS | YITGGGTTYYPDSV | ARLTDSHYVEDA |
| Kabat CDRs | (SEQ ID NO: 434) | KG | (SEQ ID NO: 436) |
| | | (SEQ ID NO: 435) | |
| KD126 | GLTFSGSD | ITGGGTT | AKARLTDSHYVED |
| IMGT CDRs | (SEQ ID NO: 437) | (SEQ ID NO: 438) | A |
| | | | (SEQ ID NO: 439) |
| KD126 variable | QVQLQESGGGLVQPGGSLRLSCAASGLTFSGSDMSWVRQAPGKG | | |
| region | PEWVSYITGGGTTYYPDSVKGRFTISRDNAKNTLYLQMNSLKPDD | | |
| (SEQ ID NO: 440) | TALYRCAKARLTDSHYVEDAQGQGTQVTVSS | | |
| KD127 | SADMS | YINTGGSSTYYADS | ARLTDSHYVEDY |
| Kabat CDRs | (SEQ ID NO: 441) | VKG | (SEQ ID NO: 443) |
| | | (SEQ ID NO: 442) | |
| KD127 | GFTFSSAD | INTGGSST | AKARLTDSHYVED |
| IMGT CDRs | (SEQ ID NO: 444) | (SEQ ID NO: 445) | Y |
| | | | (SEQ ID NO: 446) |
| KD127 variable | QVQLQESGGGLVQPGGSLRLSCAASGFTFSSADMSWVRQAPGKG | | |
| region | PEWVSYINTGGSSTYYADSVKGRFTISRDDAKSTLYLQMNSLKPE | | |
| (SEQ ID NO: 447) | DTALYRCAKARLTDSHYVEDYWGQGTQVTVSS | | |
| KD128 | SYDMS | TINTGGGTTYYADS | ARLTDSHYVEDA |
| Kabat CDRs | (SEQ ID NO: 448) | VKG | (SEQ ID NO: 450) |
| | | (SEQ ID NO: 449) | |
| KD128 | GFTFSSYD | INTGGGTT | AKARLTDSHYVED |
| IMGT CDRs | (SEQ ID NO: 451) | (SEQ ID NO: 452) | A |
| | | | (SEQ ID NO: 453) |
| KD128 variable | QVQLQESGGGMVQPGGSLRLSCAASGFTFSSYDMSWVRQAPGKG | | |
| region | PEWVSTINTGGGTTYYADSVKGRFTISRDNAKNTLYLQMNSLKPD | | |
| (SEQ ID NO: 454) | DTALYRCAKARLTDSHYVEDAWGQGTQVTVSS | | |
| KD129 | SNDMS | TIKGDGGATYYADS | ARLTDSHYVEDA |
| Kabat CDRs | (SEQ ID NO: 455) | VKG | (SEQ ID NO: 457) |
| | | (SEQ ID NO: 456) | |
| KD129 | GFTFSSND | IKGDGGAT | AKARLTDSHYVED |
| IMGT CDRs | (SEQ ID NO: 458) | (SEQ ID NO: 459) | A |
| | | | (SEQ ID NO: 460) |
| KD129 variable | QVQLQESGGGMVQPGGSLRLSCAASGFTFSSNDMSWVRQAPGKG | | |
| region | PEWVSTIKGDGGATYYADSVKGRFTISRDNAKNTLYLQMNSLKP | | |
| (SEQ ID NO: 461) | DDTALYRCAKARLTDSHYVEDAWGQGTQVTVSS | | |
| KD130 | DSDMS | YITAGGITYYADSV | ARLTETHYVEDA |
| Kabat CDRs | (SEQ ID NO: 462) | KG | (SEQ ID NO: 464) |
| | | (SEQ ID NO: 463) | |
| KD130 | GFTFSDSD | ITAGGIT | AKARLTETHYVED |
| IMGT CDRs | (SEQ ID NO: 465) | (SEQ ID NO: 466) | A |
| | | | (SEQ ID NO: 467) |
| KD130 variable | QVQLQESGGGMVHPGGSLRLSCAASGFTFSDSDMSWVRQAPGKG | | |
| region | PEWVSYITAGGITYYADSVKGRFTISRDNADHTLYLQMNSLKPED | | |
| (SEQ ID NO: 468) | TALYRCAKARLTETHYVEDAWGQGTQVTVSS | | |
| KD140 | GSDMS | YITGSGTTYYPDSV | ARLTDSHYVEDA |
| Kabat CDRs | (SEQ ID NO: 485) | KG | (SEQ ID NO: 487) |
| | | (SEQ ID NO: 486) | |
| KD140 | GFTLSGSD | ITGSGTT | AKARLTDSHYVED |
| IMGT CDRS | (SEQ ID NO: 488) | (SEQ ID NO: 489) | A |
| | | | (SEQ ID NO: 490) |
| KD140 variable | QVQLQESGGGLVQPGRSLRLSCAASGFTLSGSDMSWVRQAPGKW | | |
| region | PEWVSYITGSGTTYYPDSVKGRFTTSRDNAKNMLYLQMNSLKPD | | |
| (SEQ ID NO: 491) | DTALYRCAKARLTDSHYVEDAWGQGTQVTVSS | | |
| KD137 | GSDMS | YITGSGTTYYADTV | ARLTDSHYVEDA |
| Kabat CDRs | (SEQ ID NO: 485) | KG | (SEQ ID NO: 487) |
| | | (SEQ ID NO: 492) | |
| KD137 | GFTLSGSD | ITGSGTT | AKARLTDSHYVED |
| IMGT CDRs | (SEQ ID NO: 488) | (SEQ ID NO: 489) | A |
| | | | (SEQ ID NO: 490) |
| KD137 variable | EVQLLESGGGLVQPGGSLRLSCAASGFTLSGSDMSWVRQAPGKW | | |
| region | PEWVSYITGSGTTYYADTVKGRFTTSRDNAKNTLYLQMNSLRAE | | |
| (SEQ ID NO: 493) | DTAVYRCAKARLTDSHYVEDAWGQGTLVTVSS | | |
| Humanized | | | |
| KD140 | | | |
| KD137 heavy | EVQLLESGGGLVQPGGSLRLSCAASGFTLSGSDMSWVRQAPGKW | | |
| chain | PEWVSYITGSGTTYYADTVKGRFTTSRDNAKNTLYLQMNSLRAE | | |
| (SEQ ID NO: 494) | DTAVYRCAKARLTDSHYVEDAWGQGTLVTVSSEPKSCDKTHTCP | | |
| Humanized | PCPAPELLGGPSVFLFPPKPKDTLMISRTPEVTCVVVDVSHEDPEV | | |
| KD140 | KFNWYVDGVEVHNAKTKPREEQYNSTYRVVSVLTVLHQDWLNG | | |
| | KEYKCKVSNKALPAPIEKTISKAKGQPREPQVYTLPPSREEMTKN | | |
| | QVSLTCLVKGFYPSDIAVEWESNGQPENNYKTTPPVLDSDGSFFL | | |
| | YSKLTVDKSRWQQGNVFSCSVMHEALHNHYTQKSLSLSPGK | | |
| KD138 | GSDMS | YITGSGTTYYADSV | ARLTDSHYVEDA |
| Kabat CDRs | (SEQ ID NO: 485) | KG | (SEQ ID NO: 487) |
| | | (SEQ ID NO: 495) | |
| KD138 | GFTLSGSD | ITGSGTT | AKARLTDSHYVED |
| IMGT CDRs | (SEQ ID NO: 488) | (SEQ ID NO: 489) | A |
| | | | (SEQ ID NO: 490) |
| KD138 variable | EVQLLESGGGLVQPGRSLRLSCAASGFTLSGSDMSWVRQAPGKW | | |
| region | PEWVSYITGSGTTYYADSVKGRFTTSRDNAKNTLYLQMNSLRAE | | |
| (SEQ ID NO: 496) | DTAVYRCAKARLTDSHYVEDAWGQGTLVTVSS | | |
| Humanized | | | |
| KD140 | | | |
| KD138 heavy | EVQLLESGGGLVQPGRSLRLSCAASGFTLSGSDMSWVRQAPGKW | | |
| chain | PEWVSYITGSGTTYYADSVKGRFTTSRDNAKNTLYLQMNSLRAE | | |
| (SEQ ID NO: 497) | DTAVYRCAKARLTDSHYVEDAWGQGTLVTVSSEPKSCDKTHTCP | | |
| Humanized | PCPAPELLGGPSVFLFPPKPKDTLMISRTPEVTCVVVDVSHEDPEV | | |
| KD140 | KFNWYVDGVEVHNAKTKPREEQYNSTYRVVSVLTVLHQDWLNG | | |
| | KEYKCKVSNKALPAPIEKTISKAKGQPREPQVYTLPPSREEMTKN | | |
| | QVSLTCLVKGFYPSDIAVEWESNGQPENNYKTTPPVLDSDGSFFL | | |
| | YSKLTVDKSRWQQGNVFSCSVMHEALHNHYTQKSLSLSPGK | | |
| KD141 | GSDMS | YITGSGTTYYPDSA | ARLTDGHYVEDA |
| Kabat CDRs | (SEQ ID NO: 485) | KG | (SEQ ID NO: 499) |
| | | (SEQ ID NO: 498) | |
| KD141 | GFTFSGSD | ITGSGTT | AKARLTDGHYVED |
| IMGT CDRs | (SEQ ID NO: 500) | (SEQ ID NO: 489) | A |
| | | | (SEQ ID NO: 501) |
| KD141 | QVQLQESGGGLVQPGRSLRLSCAASGFTFSGSDMSWVRQAPGKG | | |
| variable region | PEWVSYITGSGTTYYPDSAKGRFTTSRDNAKNMLYLQMNNLKPD | | |
| (SEQ ID NO: 502) | DTALYRCAKARLTDGHYVEDAWGQGTQVTVSS | | |
| KD139 | GSDMS | YITGSGTTYYPDSV | ARLTDGHYVEDA |
| Kabat CDRs | (SEQ ID NO: 485) | KG | (SEQ ID NO: 499) |
| | | (SEQ ID NO: 486) | |
| KD139 | GFTFSGSD | ITGSGTT | AKARLTDGHYVED |
| IMGT CDRs | (SEQ ID NO: 500) | (SEQ ID NO: 489) | A |
| | | | (SEQ ID NO: 501) |

| | |
|---|---|
| KD139 variable | EVQLVESGGGLVQPGRSLRLSCAASGFTFSGSDMSWVRQAPGKG |
| region | PEWVSYITGSGTTYYPDSVKGRFTISRDNSKNTLYLQMNSLRAED |
| (SEQ ID NO: 503) | TALYRCAKARLTDGHYVEDAWGQGTLVTVSS |
| Humanized | |
| KD141 | |
| KD139 heavy | EVQLVESGGGLVQPGRSLRLSCAASGFTFSGSDMSWVRQAPGKG |
| chain | PEWVSYITGSGTTYYPDSVKGRFTISRDNSKNTLYLQMNSLRAED |
| (SEQ ID NO: 504) | TALYRCAKARLTDGHYVEDAWGQGTLVTVSSEPKSCDKTHTCPP |
| Humanized | CPAPELLGGPSVFLFPPKPKDTLMISRTPEVTCVVVDVSHEDPEVK |
| KD141 | FNWYVDGVEVHNAKTKPREEQYNSTYRVVSVLTVLHQDWLNGK |
| | EYKCKVSNKALPAPIEKTISKAKGQPREPQVYTLPPSREEMTKNQ |
| | VSLTCLVKGFYPSDIAVEWESNGQPENNYKTTPPVLDSDGSFFLYS |
| | KLTVDKSRWQQGNVFSCSVMHEALHNHYTQKSLSLSPGK |

TABLE 16
anti-TROP2 sequences
(KD006, KD007: Exemplary CDR sequences are provided)

| Anti-TROP2 | CDR1 | CDR2 | CDR3 |
|---|---|---|---|
| KD006 | TKAMG | AISESGSTYYADSV | KWYSGSFDDTRSY |
| Kabat CDRs | (SEQ ID NO: 16) | KD | DY |
| | | (SEQ ID NO: 17) | (SEQ ID NO: 18) |
| KD006 | VRTFSTKA | ISESGST | GVKWYSGSFDDTR |
| IMGT CDRs | (SEQ ID NO: 63) | (SEQ ID NO: 64) | SYDY |
| | | | (SEQ ID NO: 65) |
| KD013 | QVQLQESGGGLVQAGNSLRLSCVASVRTFSTKAMGWFRQAPGKE | | |
| (KD006 variable | REFTAAISESGSTYYADSVKDRFTISRDNVKNTVYLQMNSLKPED | | |
| region) | TAVYYCGVKWYSGSFDDTRSYDYWGQGTQVTVSS | | |
| (SEQ ID NO: 27) | | | |
| KD006 heavy | QVQLQESGGGLVQAGNSLRLSCVASVRTFSTKAMGWFRQAPGKE | | |
| chain | REFTAAISESGSTYYADSVKDRFTISRDNVKNTVYLQMNSLKPED | | |
| (SEQ ID NO: 34) | TAVYYCGVKWYSGSFDDTRSYDYWGQGTQVTVSSEPKSCDKTH | | |
| | TCPPCPAPELLGGPSVFLFPPKPKDTLMISRTPEVTCVVVDVSHEDP | | |
| | EVKFNWYVDGVEVHNAKTKPREEQYNSTYRVVSVLTVLHQDWL | | |
| | NGKEYKCKVSNKALPAPIEKTISKAKGQPREPQVYTLPPSREEMT | | |
| | KNQVSLTCLVKGFYPSDIAVEWESNGQPENNYKTTPPVLDSDGSF | | |
| | FLYSKLTVDKSRWQQGNVFSCSVMHEALHNHYTQKSLSLSPGK | | |
| KD007 | FYAMA | TITAYGNTNYADSV | NLPSASHNY |
| Kabat CDRs | (SEQ ID NO: 19) | KG | (SEQ ID NO: 21) |
| | | (SEQ ID NO: 20) | |
| KD007 | GFAFSFYA | ITAYGNT | NANLPSASHNY |
| IMGT CDRs | (SEQ ID NO: 66) | (SEQ ID NO: 67) | (SEQ ID NO: 68) |

| | |
|---|---|
| KD014 | QVQLQESGGGLVQPGGSLRLSCAASGFAFSFYAMAWYRQAPGKQ |
| (KD007 variable | RDMVATITAYGNTNYADSVKGRFTTSRDNAKNTVYLQMNSLKPE |
| region) | DTAVYVCNANLPSASHNYWGQGTQVTVSS |
| (SEQ ID NO: 28) | |
| KD007 heavy | QVQLQESGGGLVQPGGSLRLSCAASGFAFSFYAMAWYRQAPGKQ |
| chain | RDMVATITAYGNTNYADSVKGRFTTSRDNAKNTVYLQMNSLKPE |
| (SEQ ID NO: 35) | DTAVYVCNANLPSASHNYWGQGTQVTVSSEPKSCDKTHTCPPCP |
| | APELLGGPSVFLFPPKPKDTLMISRTPEVTCVVVDVSHEDPEVKEN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGK |

TABLE 17A
exemplary anti-CD47 sequences

| Anti-CD47 | CDR1 | CDR2 | CDR3 |
|---|---|---|---|
| KD070 | VNDIR | RITGGGRTDYADSV | WGRGY |
| Kabat CDRs | (SEQ ID NO: 156) | KG | (SEQ ID NO: 158) |
| | | (SEQ ID NO: 157) | |
| KD070 | RFDFSVND | ITGGGRT | WGRGY |
| IMGT CDRs | (SEQ ID NO: 159) | (SEQ ID NO: 160) | (SEQ ID NO: 161) |

| | |
|---|---|
| KD070 variable | QVQLQESGGGLVQPGGSLRLSCAASRFDFSVNDIRWYRQAPGNE |
| region | RELVARITGGGRTDYADSVKGRFTISRDNAKNTVYLQMNNLKPE |
| (SEQ ID NO: 155) | DTAVYYCWGRGYWGQGTQVTVSS |

TABLE 17B
exemplary anti-PD-1 VHHs

| Anti-PD-1 | | | |
|---|---|---|---|
| VHHs | CDR1 | CDR2 | CDR3 |
| (Kabat CDRs) | RSSMS | IISSSGIITHYSDSVK | VLDDDSGDY |
| | (SEQ ID NO: 532) | G | (SEQ ID NO: 534) |
| | | (SEQ ID NO: 533) | |
| (IMGT CDRs) | GFTFSRSS | ISSSGIIT | NAVLDDDSGDY |
| | (SEQ ID NO: 535) | (SEQ ID NO: 536) | (SEQ ID NO: 537) |

| | |
|---|---|
| variable region | QVQLQESGGGWVQPGGSLRLSCVASGFTFSRSSMSWHRQAPGKE |
| (SEQ ID | RDLVAIISSSGIITHYSDSVKGRFTISRDNAKDTVYLQMNSLKPEDT |
| NO: 538) | AVYVCNAVLDDDSGDYWGQGTQVTVSS |

TABLE 18
KD002 and variants amino acid sequence

| | SEQUENCE |
|---|---|
| | IMGT CDRs: bold and underlined |
| Code No. | FR2: twice underlined |
| KD002 heavy | QVQLQESGGGMVQVGGSLRLSCAASGFPFSSADMSWVRQAPGK |
| chain | GPEWVSYINADGSKTYYPDSVKGRFTISRDNAKNTLYLLMNNLK |
| (SEQ ID NO: 30) | PEDTALYRCARAKLTDTHYVEDYWGQGTQVTVSSEPKSCDKTH |
| | TCPPCPAPELLGGPSVFLFPPKPKDTLMISRTPEVTCVVVDVSHEDP |
| | EVKFNWYVDGVEVHNAKTKPREEQYNSTYRVVSVLTVLHQDWL |
| | NGKEYKCKVSNKALPAPIEKTISKAKGQPREPQVYTLPPSREEMT |
| | KNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| KD020 variable | EVQLVESGGGLVQPGGSLRLSCAASGFPFSSADMHWVRQAPGKG |
| region | LVWVSRINADGSKTSYADTVKGRFTISRDNAKNTLYLQMNSLRA |
| (SEQ ID NO: 71) | EDTAVYYCARAKLTDTHYVEDYWGQGTLVTVSS |
| KD021 variable | EVQLVESGGGLVQPGGSLRLSCAASGFPFSSADMSWVRQAPGKG |
| region | LEWVSYINADGSKTSYADTVKGRFTISRDNAKNTLYLQMNSLRA |
| (SEQ ID NO: 72) | EDTAVYYCARAKLTDTHYVEDYWGQGTLVTVSS |
| KD022 variable | EVQLVESGGGLVQPGGSLRLSCAASGFPFSSADMSWVRQAPGKG |
| region | PEWVSYINADGSKTYYADTVKGRFTISRDNAKNTLYLQMNSLRA |
| (SEQ ID NO: 73) | EDTAVYYCARAKLTDTHYVEDYWGQGTQVTVSS |
| KD023 variable | QVQLVESGGGLVQPGGSLRLSCAASGFPFSSADMSWVRQAPGKG |
| region | PEWVSYINADGSKTYYPDTVKGRFTISRDNAKNTLYLQMNSLRA |
| (SEQ ID NO: 74) | EDTAVYYCARAKLTDTHYVEDYWGQGTQVTVSS |
| KD024 variable | QVQLVESGGGLVQPGGSLRLSCAASGFPFSSADMSWVROAPGKG |
| region | PEWVSYINADGSKTYYPDTVKGRFTISRDNAKNTLYLQMNSLRA |
| (SEQ ID NO: 75) | EDTAVYRCARAKLTDTHYVEDYWGQGTQVTVSS |
| KD025 variable | QVQLVESGGGLVQVGGSLRLSCAASGFPFSSADMSWVRQAPGK |
| region | GPEWVSYINADGSKTYYPDTVKGRFTISRDNAKNTLYLQMNSLK |
| (SEQ ID NO: 76) | PEDTAVYRCARAKLTDTHYVEDYWGQGTQVTVSS |
| KD026 variable | QVQLQESGGGMVQVGGSLRLSCAASGFPFSSADMSWVRQAPGK |
| region | GPEWVSYINADGSKTYYPDTVKGRFTISRDNAKNTLYLQMNSLK |
| (SEQ ID NO: 77) | PEDTAVYRCARAKLTDTHYVEDYWGQGTQVTVSS |
| KD027 variable | QVQLQESGGGMVQVGGSLRLSCAASGFPFSSADMSWVRQAPGK |
| region | GPEWVSYINADGSKTYYPDSVKGRFTISRDNAKNTLYLQMNSLK |
| (SEQ ID NO: 78) | PEDTAVYRCARAKLTDTHYVEDYWGQGTQVTVSS |
| KD028 variable | EVQLLESGGGLVQPGGSLRLSCAASGFPFSSADMSWVRQAPGKG |
| region | LEWVSVINADGSKTYYADTVKGRFTISRDNSKNTLYLQMNSLRA |
| (SEQ ID NO: 79) | EDTAVYYCARAKLTDTHYVEDYWGQGTMVTVSS |
| KD029 variable | QVQLQESGGGLVQPGGSLRLSCAASGFPFSSADMSWVRQAPGKG |
| region | PEWVSYINADGSKTYYPDTVKGRFTISRDNSKNTLYLQMNNLRA |
| (SEQ ID NO: 80) | EDTALYRCARAKLTDTHYVEDYWGQGTMVTVSS |
| KD030 variable | QVQLQESGGGLVQPGGSLRLSCAASGFPFSSADMSWVRQAPGKG |
| region | LEWVSYINADGSKTYYADTVKGRFTISRDNSKNTLYLQMNSLRA |
| (SEQ ID NO: 81) | EDTAVYRCARAKLTDTHYVEDYWGQGTMVTVSS |
| KD031 variable | EVQLQESGGGLVQPGGSLRLSCAASGFPFSSADMSWVRQAPGKG |
| region | LEWVSYINADGSKTYYADTVKGRFTISRDNSKNTLYLQMNSLRA |
| (SEQ ID NO: 82) | EDTAVYRCARAKLTDTHYVEDYWGQGTMVTVSS |
| KD032 variable | QVQLLESGGGLVQPGGSLRLSCAASGFPFSSADMSWVRQAPGKG |
| region | LEWVSYINADGSKTYYADTVKGRFTISRDNSKNTLYLQMNSLRA |
| (SEQ ID NO: 83) | EDTAVYRCARAKLTDTHYVEDYWGQGTMVTVSS |
| KD033 | QVQLQESGGGLVQPGGSLRLSCAASGFPFSSADMSWVRQAPGKG |
| variable region | LEWVSVINADGSKTYYADTVKGRFTISRDNSKNTLYLQMNSLRA |
| (SEQ ID NO: 84) | EDTAVYRCARAKLTDTHYVEDYWGQGTMVTVSS |
| KD034 variable | QVQLQESGGGLVQPGGSLRLSCAASGFPFSSADMSWVRQAPGKG |
| region | LEWVSYINADGSKTYYADTVKGRFTISRDNSKNTLYLQMNSLRA |
| (SEQ ID NO: 85) | EDTAVYYCARAKLTDTHYVEDYWGQGTMVTVSS |
| KD035 variable | QVQLQESGGGLVQPGGSLRLSCAASGFPFSSADMSWVRQAPGKG |
| region | PEWVSYINADGSKTYYADTVKGRFTISRDNSKNTLYLQMNSLRA |
| (SEQ ID NO: 86) | EDTAVYRCARAKLTDTHYVEDYWGQGTMVTVSS |
| KD036 variable | QVQLQESGGGLVQPGGSLRLSCAASGFPFSSADMSWVRQAPGKG |
| region | LEWVSYINADGSKTYYPDTVKGRFTISRDNSKNTLYLQMNSLRA |
| (SEQ ID NO: 87) | EDTAVYRCARAKLTDTHYVEDYWGQGTMVTVSS |
| KD037 variable | QVQLQESGGGLVQPGGSLRLSCAASGFPFSSADMSWVRQAPGKG |
| region | LEWVSYINADGSKTYYADTVKGRFTISRDNSKNTLYLQMNNLRA |
| (SEQ ID NO: 88) | EDTAVYRCARAKLTDTHYVEDYWGQGTMVTVSS |
| KD038 variable | QVQLQESGGGLVQPGGSLRLSCAASGFPFSSADMSWVRQAPGKG |
| region | LEWVSYINADGSKTYYADTVKGRFTISRDNSKNTLYLQMNSLRA |
| (SEQ ID NO: 89) | EDTALYRCARAKLTDTHYVEDYWGQGTMVTVSS |
| KD039 variable | QVQLQESGGGMVQPGGSLRLSCAASGFPFSSADMSWVRQAPGK |
| region | GLEWVSYINADGSKTYYADTVKGRFTISRDNSKNTLYLQMNSLR |
| (SEQ ID NO: 90) | AEDTAVYRCARAKLTDTHYVEDYWGQGTQVTVSS |
| KD040 variable | QVQLQESGGGLVQPGGSLRLSCAASGFPFSSADMSWVRQAPGKQ |
| region | REWVSYINADGSKTYYADTVKGRFTISRDNSKNTLYLQMNSLRA |
| (SEQ ID NO: 91) | EDTAVYRCARAKLTDTHYVEDYWGQGTMVTVSS |
| KD041 variable | QVQLQESGGGLVQPGGSLRLSCAASGFPFSSADMSWVRQAPGKG |
| region | LEWVSYINAEGSKTYYADTVKGRFTISRDNSKNTLYLQMNSLRA |
| (SEQ ID NO: 92) | EDTAVYRCARAKLTDTHYVEDYWGQGTMVTVSS |
| KD042 variable | QVQLQESGGGLVQPGGSLRLSCAASGFPFSSADMSWVRQAPGKG |
| region | LEWVSYINANGSKTYYADTVKGRFTISRDNSKNTLYLQMNSLRA |
| (SEQ ID NO: 93) | EDTAVYRCARAKLTDTHYVEDYWGQGTMVTVSS |

TABLE 19
KD005 and variants amino acid sequence

| | SEQUENCE |
|---|---|
| | IMGT CDRs: bold and underlined |
| Code | FR2: twice underlined |
| KD005 heavy | QVQLQESGGGLVEAGGSLRLSCAASGFTFSGSDMSWVRQAPGK |
| chain | GPEWVSYITSGGTTYYPDSVKGRFTISRDNAKNTLYLQMNSLKP |
| (SEQ ID NO: 33) | DDTALYRCAKSRLTDSHYVEDAWGQGTQVTVSSEPKSCDKTHT |
| | CPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSREEMTK |
| | NQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| KD043 variable | EVQLLESGGGLVQPGGSLRLSCAASGFTFSGSDMSWVRQAPGKG |
| region | LEWVSAITSGGTTYYADTVKGRFTISRDNSKNTLYLQMNSLRAE |
| (SEQ ID NO: 94) | DTAVYYCAKSRLTDSHYVEDAWGQGTLVTVSS |
| KD044 variable | EVQLLESGGGLVQPGGSLRLSCAASGFTFSGSDMSWVRQAPGKG |
| region | LEWVSYITSGGTTYYADTVKGRFTISRDNSKNTLYLQMNSLRAE |
| (SEQ ID NO: 95) | DTAVYYCAKSRLTDSHYVEDAWGQGTLVTVSS |
| KD045 variable | EVQLLESGGGLVQPGGSLRLSCAASGFTFSGSDMSWVRQAPGKG |
| region | LEWVSYITSGGTTYYADTVKGRFTISRDNSKNTLYLQMNSLRAE |
| (SEQ ID NO: 96) | DTAVYRCAKSRLTDSHYVEDAWGQGTLVTVSS |
| KD046 variable | QVQLLESGGGLVQPGGSLRLSCAASGFTFSGSDMSWVRQAPGKG |
| region | PEWVSYITSGGTTYYADTVKGRFTISRDNSKNTLYLQMNSLRAE |
| (SEQ ID NO: 97) | DTAVYYCAKSRLTDSHYVEDAWGQGTLVTVSS |
| KD047 variable | QVQLLESGGGLVQPGGSLRLSCAASGFTFSGSDMSWVRQAPGKG |
| region (SEQ ID | PEWVSYITSGGTTYYADTVKGRFTISRDNSKNTLYLQMNSLRAE |
| NO: 98) | DTAVYRCAKSRLTDSHYVEDAWGQGTLVTVSS |
| KD048 variable | QVQLQESGGGLVQPGGSLRLSCAASGFTFSGSDMSWVRQAPGKG |
| region (SEQ ID | PEWVSYITSGGTTYYPDTVKGRFTISRDNSKNTLYLQMNSLRAED |
| NO: 99) | TAVYYCAKSRLTDSHYVEDAWGQGTQVTVSS |
| KD049 variable | QVQLQESGGGLVQPGGSLRLSCAASGFTFSGSDMSWVRQAPGKG |
| region | PEWVSYITSGGTTYYPDTVKGRFTISRDNSKNTLYLQMNSLRAED |
| (SEQ ID NO: 100) | TAVYRCAKSRLTDSHYVEDAWGQGTQVTVSS |
| KD050 variable | QVQLQESGGGLVQPGGSLRLSCAASGFTFSGSDMSWVRQAPGKG |
| region | PEWVSYITSGGTTYYPDTVKGRFTISRDNSKNTLYLQMNSLKPDD |
| (SEQ ID NO: 101) | TAVYRCAKSRLTDSHYVEDAWGQGTQVTVSS |
| KD051 variable | QVQLQESGGGLVQPGGSLRLSCAASGFTFSGSDMSWVRQAPGKG |
| region | PEWVSYITSGGTTYYPDTVKGRFTISRDNAKNTLYLQMNSLKPD |
| (SEQ ID NO: 102) | DTALYRCAKSRLTDSHYVEDAWGQGTQVTVSS |
| KD052 variable | EVQLVESGGGLIQPGGSLRLSCAASGFTFSGSDMSWVRQAPGKG |
| region | LEWVSVITSGGTTYYADTVKGRFTISRDNSKNTLYLQMNSLRAE |
| (SEQ ID NO: 103) | DTAVYYCAKSRLTDSHYVEDAWGQGTLVTVSS |
| KD053 variable | QVQLQESGGGLIQPGGSLRLSCAASGFTFSGSDMSWVRQAPGKG |
| region | PEWVSYITSGGTTYYPDTVKGRFTISRDNSKNTLYLQMNSLRAD |
| (SEQ ID NO: 104) | DTALYRCAKSRLTDSHYVEDAWGQGTLVTVSS |
| KD054 variable | QVQLQESGGGLIQPGGSLRLSCAASGFTFSGSDMSWVRQAPGKG |
| region | LEWVSYITSGGTTYYADTVKGRFTISRDNSKNTLYLQMNSLRAE |
| (SEQ ID NO: 105) | DTAVYRCAKSRLTDSHYVEDAWGQGTLVTVSS |
| KD055 variable | EVQLQESGGGLIQPGGSLRLSCAASGFTFSGSDMSWVRQAPGKG |
| region | LEWVSYITSGGTTYYADTVKGRFTISRDNSKNTLYLQMNSLRAE |
| (SEQ ID NO: 106) | DTAVYRCAKSRLTDSHYVEDAWGQGTLVTVSS |
| KD056 variable | QVQLVESGGGLIQPGGSLRLSCAASGFTFSGSDMSWVRQAPGKG |
| region | LEWVSYITSGGTTYYADTVKGRFTISRDNSKNTLYLQMNSLRAE |
| (SEQ ID NO: 107) | DTAVYRCAKSRLTDSHYVEDAWGQGTLVTVSS |
| KD057 variable | QVQLQESGGGLIQPGGSLRLSCAASGFTFSGSDMSWVRQAPGKG |
| region | LEWVSVITSGGTTYYADTVKGRFTISRDNSKNTLYLQMNSLRAE |
| (SEQ ID NO: 108) | DTAVYRCAKSRLTDSHYVEDAWGQGTLVTVSS |
| KD058 variable | QVQLQESGGGLIQPGGSLRLSCAASGFTFSGSDMSWVRQAPGKG |
| region | LEWVSYITSGGTTYYADTVKGRFTISRDNSKNTLYLQMNSLRAE |
| (SEQ ID NO: 109) | DTAVYYCAKSRLTDSHYVEDAWGQGTLVTVSS |
| KD059 variable | QVQLQESGGGLIQPGGSLRLSCAASGFTFSGSDMSWVRQAPGKG |
| region | PEWVSYITSGGTTYYADTVKGRFTISRDNSKNTLYLQMNSLRAE |
| (SEQ ID NO: 110) | DTAVYRCAKSRLTDSHYVEDAWGQGTLVTVSS |
| KD060 variable | QVQLQESGGGLIQPGGSLRLSCAASGFTFSGSDMSWVRQAPGKG |
| region | LEWVSYITSGGTTYYPDTVKGRFTISRDNSKNTLYLQMNSLRAED |
| (SEQ ID NO: 111) | TAVYRCAKSRLTDSHYVEDAWGQGTLVTVSS |
| KD061 variable | QVQLQESGGGLIQPGGSLRLSCAASGFTFSGSDMSWVRQAPGKG |
| region | LEWVSYITSGGTTYYADTVKGRFTISRDNSKNTLYLQMNSLRAD |
| (SEQ ID NO: 112) | DTAVYRCAKSRLTDSHYVEDAWGQGTLVTVSS |
| KD062 variable | QVQLQESGGGLIQPGGSLRLSCAASGFTFSGSDMSWVRQAPGKG |
| region | LEWVSYITSGGTTYYADTVKGRFTISRDNSKNTLYLQMNSLRAE |
| (SEQ ID NO: 113) | DTALYRCAKSRLTDSHYVEDAWGQGTLVTVSS |
| KD063 variable | QVQLQESGGGLIEPGGSLRLSCAASGFTFSGSDMSWVRQAPGKG |
| region | LEWVSYITSGGTTYYADTVKGRFTISRDNSKNTLYLQMNSLRAE |
| (SEQ ID NO: 114) | DTAVYRCAKSRLTDSHYVEDAWGQGTQVTVSS |
| KD064 variable | QVQLQESGGGLIQPGGSLRLSCAASGFTFSGSDMSWYRQAPGKG |
| region | REWVSYITSGGTTYYADTVKGRFTISRDNSKNTLYLQMNSLRAE |
| (SEQ ID NO: 115) | DTAVYRCAKSRLTDSHYVEDAWGQGTLVTVSS |

Table 20A-D: Alignment of IMGT CDRs or FR2 sequences of KD001-KD005 and variants

TABLE 20A

| | |
|---|---|
| IMGT | KD001-KD005 original sequences |
| CDRH1 | GFTFSGSD (SEQ ID NOs: 52, 59 and 62) |
| | GFPFSSAD (SEQ ID NO: 55) |
| | GFTFSNYD (SEQ ID NO: 56) |
| | GFTLSGSD (SEQ ID NO: 488) |
| IMGT | Alignment |
| CDRH1 | 52 GFTFSGSD 8 |
| Alignment, | 56 GFTFSNYD 8 |
| Consensus, | 55 GFPFSSAD 8 |
| Examples | 488 GFTLSGSD 8 |
| | Consensus |
| | GFX₇ₐX_{7d}SX_{7b}X_{7c}D (SEQ ID NO: 122) |
| | Examples |
| | a) Wherein any one of X₇ₐ to X_{7d} is any amino acid. |
| | b) Wherein X₇ₐ is any amino acid; |
| | Wherein X_{7b} is G, N, S or D; |
| | Wherein X_{7c} is any amino acid and/or |
| | Wherein X_{7d} is F, V, L, I, or M. |
| | (SEQ ID NO: 181) |
| | c) Wherein X₇ₐ is T or P; |
| | Wherein X_{7b} is G, N or S; |
| | Wherein X_{7c} is S, Y or A and/or |
| | Wherein X_{7d} is F or L. |
| | (SEQ ID NO: 182) |

TABLE 20B

| | |
|---|---|
| IMGT | KD001-KD005 original sequences |
| CDRH2 | INADGSKT (SEQ ID NO: 47) |
| | ITSGGTT (SEQ ID NOs: 50 and 53) |
| | INAGGSNT (SEQ ID NO: 57) |
| | ITGSGTT (SEQ ID NO: 60) |
| | KD002 Variants |
| | INANGSKT (SEQ ID NO: 123) |
| | INAEGSKT (SEQ ID NO: 124) |
| IMGT | Alignment |
| CDRH2 | 47 INADGSKT 8 |
| Alignment, | 124 INANGSKT 8 |
| Consensus, | 123 INAEGSKT 8 |
| Examples | 57 INAGGSNT 8 |
| | 50 ITSGGTT- 7 |
| | 60 ITGSGTT- 7 |
| | Consensus |
| | IX₈ₐX_{8b}X_{8c}GX_{8d}X₈ₑX_{8f} (SEQ ID NO: 125) |
| | Examples |
| | a) Wherein any one of X₈ₐ to X_{8f} is any amino acid. |
| | b) Wherein X₈ₐ is S, T, Nor K; |
| | Wherein X_{8b} is A, S or G; |
| | Wherein X_{8c} is any amino acid; |
| | Wherein X_{8d} is S, T or A; |
| | Wherein X₈ₑ is S, T, N or K and/or |
| | Wherein X_{8f} is any amino acid or absent. (SEQ ID NO: 183) |
| | c) Wherein X₈ₐ is N or T; |
| | Wherein X_{8b} is A, S or G; |
| | Wherein X_{8c} is D, N, E, G or S; |
| | Wherein X_{8d} is S or T; |
| | Wherein X₈ₑ is K, N or T and/or |
| | Wherein X_{8f} is T or absent. (SEQ ID NO: 184) |

TABLE 20C

| | |
|---|---|
| IMGT | KD001-KD005 original sequences |
| CDRH3 | ARAKLTDTHYVEDY (SEQ ID NOs: 48 and 58) |
| | AKSRLTDSHYVEDA (SEQ ID NO: 51) |
| | AKARLTDSHYVEDA (SEQ ID NO: 54 and 61) |
| | AKARLTDGHYVEDA (SEQ ID NO: 432) |
| IMGT | Alignment |
| CDRH3 | 48 ARAKLTDTHYVEDY 14 |
| Alignment, | 51 AKSRLTDSHYVEDA 14 |
| Consensus, Examples | 54 AKARLTDSHYVEDA 14 |
| | 61 AKARLTDSHYVEDA 14 |
| | 432 AKARLTDGHYVEDA 14 |
| | *:::*** ***** |
| | Consensus |
| | AX₉ₐX_{9b}X_{9c}LTDX_{9d}HYVEDX₉ₑ (SEQ ID NO: 126) |
| | Examples |
| | a) Wherein any one of X₉ₐ to X₉ₑ is any amino acid. |
| | b) Wherein X₉ₐ is Q, H, R or K; |
| | Wherein X_{9b} is S, T or A; |
| | Wherein X_{9c} is Q, H, R or K; |
| | Wherein X_{9d} is S, T or A or any amino acid and/or |
| | Wherein X₉ₑ is any amino acid or absent. |
| | (SEQ ID NO: 185) |
| | c) Wherein X₉ₐ is R or K; |
| | Wherein X_{9b} is A or S; |
| | Wherein X_{9c} is K or R; |
| | Wherein X_{9d} is T, S or G and/or |
| | Wherein X₉ₑ is Y, A or absent. |
| | (SEQ ID NO: 186) |

TABLE 20D

| | |
|---|---|
| IMGT FR2 | KD001-KD005 original sequences (e.g., |
| | aa 34-50 of SEQ ID NO: 33) |
| | MSWVRQAPGKGPEWVSY (SEQ ID: 116) |
| | KD002 variants |
| | MSWVRQAPGKGLEWVSY (SEQ ID: 117) |
| | MSWVRQAPGKGLEWVSV (SEQ ID: 118) |
| | MSWVRQAPGKQREWVSY (SEQ ID: 119) |
| | KD005 variants |
| | MSWVRQAPGKGLEWVSA (SEQ ID: 120) |
| | MSWVRQAPGKGLEWVSV (SEQ ID: 118) |
| | MSWVRQAPGKGLEWVSY (SEQ ID: 117) |
| | Other sequences |
| | MSWVRQAPGKGPEWLST (SEQ ID NO: 354) |
| | MSCVRQAPGTGPEWVSY (SEQ ID NO: 355) |
| | MSWVRQVPGQGPEWVSS (SEQ ID NO: 356) |
| | MSWVRQAPGKGPEWVST (SEQ ID NO: 360) |
| | MSWVRQAPGKGPEWGSY (SEQ ID NO: 361) |
| | MSWVRQAPGKWPEWVSY (SEQ ID NO: 505) |
| IMGT FR2 | Alignment |
| Alignment, | 119 MSWVRQAPGKQREWVSY 17 |
| Consensus, Examples | 120 MSWVRQAPGKGLEWVSA 17 |
| | 118 MSWVRQAPGKGLEWVSV 17 |
| | 116 MSWVRQAPGKGPEWVSY 17 |
| | 117 MSWVRQAPGKGLEWVSY 17 |
| | ********** **** |
| | Consensus |
| | MSWVRQAPGKX₆ₐX_{6b}EWVSX_{6c} (consensus SEQ ID NO: 121) |
| | Examples |
| | a) Wherein any one of X₆ₐ to X_{6c} is any amino acid. |
| | b) Wherein X₆ₐ is Q, G or W; |
| | Wherein X_{6b} is R, L or P and/or |
| | Wherein X_{6c} is Y, A or V. |
| | (SEQ ID NO: 180) |

Table 21A-C: Alignment of Kabat CDRs or FR2 sequences of KD001-KD005 and variants

TABLE 21A

| | |
|---|---|
| Kabat | KD001-KD005 original sequences |
| CDRH1 | GSDMS (SEQ ID NOs: 1, 10 and 13) |
| | SADMS (SEQ ID NO: 4) |
| | NYDMS (SEQ ID NO: 7) |
| | KD002 variants |
| | SADMH (SEQ ID NO: 127) |
| Kabat | Alignment |
| CDRH1 | 7 NYDMS 5 |
| Alignment, | 1 GSDMS 5 |
| Consensus, Examples | 4 SADMS 5 |
| | 127 SADMH 5 |
| | .** |
| | Consensus |
| | X₁₀ₐX_{10b}DMX_{10c} (SEQ ID NO: 128) |
| | Examples |
| | a) Wherein any one of X₁₀ₐ to X_{10c} is any amino acid. |
| | b) Wherein X₁₀ₐ is S, G, N or D; |
| | Wherein X_{10b} is any amino acid and/or |
| | Wherein X_{10c} is any amino acid. |
| | (SEQ ID NO: 187) |
| | c) Wherein X₁₀ₐ is N, G or S; |
| | Wherein X_{10b} is Y, S or A and/or |
| | Wherein X_{10c} is S or H. |
| | (SEQ ID NO: 188) |

TABLE 21B

| | |
|---|---|
| Kabat | KD001-KD005 original sequences |
| CDRH2 | YITSGGTTYYPDSVKG (SEQ ID NOs: 2 and 14) |
| | YINADGSKTYYPDSVKG (SEQ ID NO: 5) |
| | YINAGGSNTDYPDSVKG (SEQ ID NO: 8) |
| | YITGSGTTYYPDSVKG (SEQ ID NO: 11) |
| | KD002 variants |
| | RINADGSKTSYADTVKG (SEQ ID NO: 129) |
| | YINADGSKTSYADTVKG (SEQ ID NO: 130) |
| | YINADGSKTYYPDTVKG (SEQ ID NO: 131) |
| | VINADGSKTYYADTVKG (SEQ ID NO: 132) |
| | YINADGSKTYYADTVKG (SEQ ID NO: 133) |
| | YINAEGSKTYYADTVKG (SEQ ID NO: 134) |
| | YINANGSKTYYADTVKG (SEQ ID NO: 135) |
| | KD005 variants |
| | AITSGGTTYYADTVKG (SEQ ID NO: 136) |
| | YITSGGTTYYADTVKG (SEQ ID NO: 137) |
| | YITSGGTTYYPDTVKG (SEQ ID NO: 138) |
| | VITSGGTTYYADTVKG (SEQ ID NO: 139) |
| Kabat | Alignment |
| CDRH2 | 11 YI-TGSGTTYYPDSVKG 16 |
| Alignment, | 2 YI-TSGGTTYYPDSVKG 16 |
| Consensus, | 138 YI-TSGGTTYYPDTVKG 16 |
| Examples | 136 AI-TSGGTTYYADTVKG 16 |
| | 135 YI-TSGGTTYYADTVKG 16 |
| | 139 VI-TSGGTTYYADTVKG 16 |
| | 8 YINAGGSNTDYPDSVKG 17 |
| | 129 RINADGSKTSYADTVKG 17 |
| | 130 YINADGSKTSYADTVKG 17 |
| | 5 YINADGSKTYYPDSVKG 17 |
| | 131 YINADGSKTYYPDTVKG 17 |
| | 135 YINANGSKTYYADTVKG 17 |
| | 134 YINAEGSKTYYADTVKG 17 |
| | 132 VINADGSKTYYADTVKG 17 |
| | 133 YINADGSKTYYADTVKG 17 |
| | *:...* * *:*** |
| | Consensus |
| | X₁₁ₐIX_{11b}X_{11c}X_{11d}X₁₁ₑX_{11f}X_{11g}TX₁₁ₕYX₁₁ᵢDX₁₁ⱼVKG (SEQ ID NO: 140) |
| | Examples |
| | a) Wherein any one of X₁₁ₐ to X₁₁ⱼ is any amino acid. |
| | b) Wherein X₁₁ₐ is any amino acid; |
| | Wherein X_{11b} is any amino acid or absent; |
| | Wherein X_{11c} is S, T or A; |
| | Wherein X_{11d} is any amino acid; |
| | Wherein X₁₁ₑ is S, A or G; |
| | Wherein X_{11f} S, A or G; |
| | Wherein X_{11g} is S, T, N or K; |
| | Wherein X₁₁ₕ is any amino acid; |
| | Wherein X₁₁ᵢ is any amino acid; |
| | Wherein X₁₁ⱼ is S, T, or A. |
| | (SEQ ID NO: 189) |
| | c) Wherein X₁₁ₐ is Y, A, V or R; |
| | Wherein X_{11b} is absent or N; |
| | Wherein X_{11c} is T or A; |
| | Wherein X_{11d} is G, S, D, N or E; |
| | Wherein X₁₁ₑ is S or G; |
| | Wherein X_{11f} is G or S; |
| | Wherein X_{11g} is T, N or K; |
| | Wherein X₁₁ₕ is Y, D or S; |
| | Wherein X₁₁ᵢ is P or A and/or |
| | Wherein X₁₁ⱼ is S or T. |
| | (SEQ ID NO: 190) |

TABLE 21C

| | |
|---|---|
| Kabat | KD001-KD005 original sequences |
| CDRH3 | ARLTDSHYVED (SEQ ID NO: 3) |
| | AKLTDTHYVEDY (SEQ ID NOs: 6 and 9) |
| | ARLTDSHYVEDA (SEQ ID NO: 12) |
| | SRLTDSHYVED (SEQ ID NO: 15) |
| | KD005 variants |
| | SRLTDSHYVEDA (SEQ ID NO: 141) |
| Kabat | Alignment |
| CDRH3 | 6 AKLTDTHYVEDY 12 |
| Alignment, | 3 ARLTDSHYVED-11 |
| Consensus, Examples | 12 ARLTDSHYVEDA 12 |
| | 15 SRLTDSHYVED-11 |
| | 140 SRLTDSHYVEDA 12 |
| | ::***.***** |
| | Consensus |
| | X₁₂ₐX_{12b}LTDX_{12c}HYVEDX_{12d} (SEQ ID NO: 142) |
| | Examples |
| | a) Wherein any one of X₁₂ₐ to X_{12d} is any amino acid. |
| | b) Wherein X₁₂ₐ is S, T or A; |
| | Wherein X_{12b} is Q, H, R or K; |
| | Wherein X_{12c} is S, T or A and/or |
| | Wherein X_{12d} is any amino acid or absent. |
| | (SEQ ID NO: 191) |
| | c) Wherein X₁₂ₐ is A or S; |
| | Wherein X_{12b} is K or R; |
| | Wherein X_{12c} is T or S and/or |
| | Wherein X_{12d} is Y, A or absent. |
| | (SEQ ID NO: 192) |

TABLE 21D

| | |
|---|---|
| Kabat FR2 | WVRQAPGKGPEWVS (SEQ ID NO: 170) |
| | WVRQAPGKGLEWVS (SEQ ID NO: 171) |
| | WVRQAPGKQREWVS (SEQ ID NO: 172) |
| | WYRQAPGKGREWVS (SEQ ID NO: 173) |
| | WVRQAPGKGLVWVS (SEQ ID NO: 174) |
| | WVRQAPGKGPEWLS (SEQ ID NO: 357) |
| | CVRQAPGTGPEWVS (SEQ ID NO: 358) |
| | WVRQVPGQGPEWVS (SEQ ID NO: 359) |
| Kabat FR2 | Alignment |
| Alignment, | 173 WYRQAPGKGREWVS |
| Consensus, Examples | 172 WVRQAPGKQREWVS |
| | 174 WVRQAPGKGLVWVS |
| | 170 WVRQAPGKGPEWVS |
| | 171 WVRQAPGKGLEWVS |
| | * ****** *** |
| | Consensus |
| | WX₁₃ₐRQAPGKX_{13b}X_{13c}X_{13d}WVS (SEQ ID NO: 175) |
| | Examples |
| | a) Wherein any one of X₁₃ₐ to X_{13d} is any amino acid. |
| | b) Wherein X₁₃ₐ is V or Y; |
| | wherein X_{13b} is G or Q; |
| | wherein X_{13b} is P, L or R and; |
| | wherein X_{13d} is E or V |
| | (SEQ ID NO: 193) |
| | c) Wherein X₁₃ₐ is V; |
| | wherein X_{13b} is G or Q and; |
| | wherein X_{13b} is P, L or R |
| | wherein X_{13d} is E |
| | (SEQ ID NO: 194) |

SEQ ID NO: 36 Linker-Human IgG1 hinge AA sequences:
EPKSCDKTHTCPPCP
SEQ ID NO: 481: Linker
EPKSSDKTHTCPPCP
SEQ ID NO: 37 Human IgG1 CH2 AA sequences:
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAK
SEQ ID NO: 38 Human IgG1 CH3 AA sequences:
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 39 human TROP2
MARGPGLAPPPLRLPLLLLVLAAVTGHTAAQDNCTCPTNKMTVCSPDGPGGRCQCRAL
GSGMAVDCSTLTSKCLLLKARMSAPKNARTLVRPSEHALVDNDGLYDPDCDPEGRFKA
RQCNQTSVCWCVNSVGVRRTDKGDLSLRCDELVRTHHILIDLRHRPTAGAFNHSDLDA
ELRRLFRERYRLHPKFVAAVHYEQPTIQIELRQNTSQKAAGDVDIGDAAYYFERDIKGES
LFQGRGGLDLRVRGEPLQVERTLIYYLDEIPPKFSMKRLTAGLIAVIVVVVVALVAGMA
VLVITNRRKSGKYKKVEIKELGELRKEPSL
SEQ ID NO: 40 cynomolgus TROP2
MARGPGLAPPPLRLPLLLLLLAAVTGHTAAQDNCTCPTNKMTVCSPDGPGGRCQCRAL
GSGVAVDCSTLTSKCLLLKARMSAPKNARTLVRPNEHALVDNDGLYDPDCDPEGRFKA
RQCNQTSVCWCVNSVGVRRTDKGDLSLRCDELVRTHHILIDLRHRPTAGAFNHSDLDA
ELRRLFRERYRLHPKFVAAVHYEQPTIQIELRQNTSQKAAGDVDIGDAAYYFERDVKGE
SLFQGRGGLDLRVRGEPLQVERTLIYYLDEIPPKFSMKRLTAGLIAVIVVVVVALVAGVA
VLVISNRRKSGKYKKVEIKELGELRKEPSL
SEQ ID NO: 41 mouse TROP2
MARGLDLAPLLLLLLAMATRFCTAQSNCTCPTNKMTVCDTNGPGGVCQCRAMGSQVL
VDCSTLTSKCLLLKARMSARKSGRSLVMPSEHAILDNDGLYDPECDDKGRFKARQCNQ
TSVCWCVNSVGVRRTDKGDQSLRCDEVVRTHHILIELRHRPTDRAFNHSDLDSELRRLF
QERYKLHPSFLSAVHYEEPTIQIELRQNASQKGLRDVDIADAAYYFERDIKGESLFMGRR
GLDVQVRGEPLHVERTLIYYLDEKPPQFSMKRLTAGVIAVIAVVSVAVVAGVVVLVVT
KRRKSGKYKKVELKELGEMRSEPSL
SEQ ID NO: 42 Linker-Human IgG4 hinge AA sequences:
ESKYGPPCPPCP
SEQ ID NO: 179 Human IgG4 CH2 AA sequences:
APEAAGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAK
SEQ ID NO: 43 Human IgG4 CH3 AA sequences:
GQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 143 (KD002 consensus)
X₁ₐVQLX_{1b}ESGGGX_{1c}VQX_{1d}GGSLRLSCAASGFPFSSADMSWVRQAPGKX₁ₑX_{1f}EWVSX_{1g}
INAXINGSKTYYX₁ᵢDX₁ⱼVKGRFTISRDNX₁ₖKNTLYLX₁ₗMNX₁ₘLX₁ₙX₁ₒEDTAX₁ₚYX_{1q}CA
RAKLTDTHYVEDYWGQGTX₁ᵣVTVSS
SEQ ID NO: 144 (KD005 consensus)
X₂ₐVQLX_{2b}ESGGGLX_{2c}X_{2d}X₂ₑGGSLRLSCAASGFTFSGSDMSWVRQAPGKGX_{2f}EWVSX_{2g}
ITSGGTTYYX₂ₕDX₂ᵢVKGRFTISRDNX₂ⱼKNTLYLQMNSLX₂ₖX₂ₗX₂ₘDTAX₂ₙYX₂ₒCAKSRL
TDSHYVEDAWGQGTX₂ₚVTVSS
SEQ ID NO: 145 (Consensus from KD001 to KD005)
QVQLQESGGGX₃ₐVX_{3b}X_{3c}GX_{3d}SLRLSCAAS [symbol] DMSWVRQAPGKGPEWVSY [symbol]
X₃ₙYPDSVKGRFTX₃ₒSRDNAKNX₃ₚLYLX_{3q}MNX₃ᵣLX₃ₛPX₃ₜDTALYR
C [symbol] WGQGTQVTVSS
SEQ ID NO: 146 (Consensus for KD001-KD005 and selected variants)
X₄ₐVQLX_{4b}ESGGGX_{4c}X_{4d}X₄ₑX_{4f}GX_{4g}SLRLSCAASGFX₄ₕFSX₄ᵢX₄ⱼDMX₄ₖWX₄ₗRQAPGKX₄ₘ
X₄ₙX₄ₒWVSX₄ₚIX_{4q}X₄ᵣX₄ₛGX₄ₜX₄ᵤTX₄ᵥYX_{4w}DX₄ₓVKGRFTX_{4y}SRDNX_{4z}KNX₅ₐLYLX_{5b}MNX_{5c}
LX_{5d}X₅ₑX_{5f}DTAX_{5g}YX₅ₕCAX₅ᵢX₅ⱼX₅ₖLTDX₅ₗHYVEDX₅ₘWGQGTX₅ₙVTVSS.
SEQ ID NO: 147 (TROP2 delta 27-36)
MARGPGLAPPPLRLPLLLLVLAAVTGPTNKMTVCSPDGPGGRCQCRALGSGMAVDCST
LTSKCLLLKARMSAPKNARTLVRPSEHALVDNDGLYDPDCDPEGRFKARQCNQTSVCW
CVNSVGVRRTDKGDLSLRCDDLVRTHHILIDLRHRPTAGAFNHSDLDAELRRLFRERYR
LHPKFVAAVHYEQPTIQIELRQNTSQKAAGEVDIGDAAYYFERDIKGESLFQGRGGLDL
RVRGEPLQVERTLIYYLDEIPPKFSMKRLTAGLIAVIVVVVVALVAGMAVLVITNRRKSG
KYKKVEIKELGELRKEPSL
SEQ ID NO: 148 (TROP2 delta 27-44)
MARGPGLAPPPLRLPLLLLVLAAVTGSPDGPGGRCQCRALGSGMAVDCSTLTSKCLLLK
ARMSAPKNARTLVRPSEHALVDNDGLYDPDCDPEGRFKARQCNQTSVCWCVNSVGVR
RTDKGDLSLRCDDLVRTHHILIDLRHRPTAGAFNHSDLDAELRRLFRERYRLHPKFVAA
VHYEQPTIQIELRQNTSQKAAGEVDIGDAAYYFERDIKGESLFQGRGGLDLRVRGEPLQ
VERTLIYYLDEIPPKFSMKRLTAGLIAVIVVVVVALVAGMAVLVITNRKSGKYKKVEIKE
LGELRKEPSL
SEQ ID NO: 149 (TROP2 delta 27-56)
MARGPGLAPPPLRLPLLLLVLAAVTGALGSGMAVDCSTLTSKCLLLKARMSAPKNART
LVRPSEHALVDNDGLYDPDCDPEGRFKARQCNQTSVCWCVNSVGVRRTDKGDLSLRC
DDLVRTHHILIDLRHRPTAGAFNHSDLDAELRRLFRERYRLHPKFVAAVHYEQPTIQIEL
RQNTSQKAAGEVDIGDAAYYFERDIKGESLFQGRGGLDLRVRGEPLQVERTLIYYLDEIP
PKFSMKRLTAGLIAVIVVVVVALVAGMAVLVITNRRKSGKYKKVEIKELGELRKEPSL
SEQ ID NO: 150 (KD065: TROP2-CD47 bispecific, TROP2 variable region at the N-terminal-
variable regions underlined)
QVQLQESGGGLVQPGRSLRLSCAASGFTESGSDMSWVRQAPGKGPEWVSYITGSGTTY
YPDSVKGRFTTSRDNAKNMLYLOMNSLKPDDTALYRCAKARLTDSHYVEDAWGQGT
QVTVSSEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSL
SPGKAPAPAPAPAPAPAPAPAPAPKAQVQLQESGGGLVQPGGSLRLSCAASREDESVNDI
RWYRQAPGNERELVARITGGGRTDYADSVKGRFTISRDNAKNTVYLOMNNLKPEDTA
VYYCWGRGYWGQGTQVTVSS
SEQ ID NO: 151 (KD066: TROP2-CD47 bispecific, TROP2 variable region at the C-terminal--
variable regions underlined)
QVQLQESGGGLVQPGGSLRLSCAASREDESVNDIRWYRQAPGNERELVARITGGGRTD
YADSVKGRFTISRDNAKNTVYLQMNNLKPEDTAVYYCWGRGYWGOGTQVTVSSEPKS
CDKTHTCPPCPAPELLGGPSVFLFPPKPKDTLMISRTPEVTCVVVDVSHEDPEVKENWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGKAPAPAPA
PAPAPAPAPAPAPKAQVQLQESGGGLVQPGRSLRLSCAASGFTESGSDMSWVRQAPGK
GPEWVSYITGSGTTYYPDSVKGRFTTSRDNAKNMLYLOMNSLKPDDTALYRCAKARLT
DSHYVEDAWGOGTQVTVSS
SEQ ID NO: 152: KD067 (variable region 1-140-mouse hinge-mouse constant region-IMGT
CDRH1, CDRH2 and CDRH3 in bold and underlined)
QVQLQESGGGMVQVGGSLRLSCAASGFPFSSADMSWVRQAPGKGPEWVSYINADGSK
TYYPDSVKGRFTISRDNAKNTLYLLMNNLKPEDTALYRCARAKLTDTHYVEDYWGQG
TQVTVSSVPRDCGCKPCICTVPEVSSVFIFPPKPKDVLTITLTPKVTCVVVDISKDDPEVQ
FSWFVDDVEVHTAQTQPREEQFNSTFRSVSELPIMHQDWLNGKEFKCRVNSAAFPAPIE
KTISKTKGRPKAPQVYTIPPPKEQMAKDKVSLTCMITDFFPEDITVEWQWNGQPAENYK
NTQPIMNTNGSYFVYSKLNVQKSNWEAGNTFTCSVLHEGLHNHHTEKSLSHSPGK
SEQ ID NO: 153: KD068 (variable region 1-139-mouse hinge-mouse constant region-IMGT
CDRH1, CDRH2 and CDRH3 in bold and underlined)
QVQLQESGGGLVEAGGSLRLSCAASGFTFSGSDMSWVRQAPGKGPEWVSYITSGGTT
YYPDSVKGRFTISRDNAKNTLYLQMNSLKPDDTALYRCAKSRLTDSHYVEDAWGQGT
QVTVSSVPRDCGCKPCICTVPEVSSVFIFPPKPKDVLTITLTPKVTCVVVDISKDDPEVQFS
WFVDDVEVHTAQTQPREEQFNSTFRSVSELPIMHQDWLNGKEFKCRVNSAAFPAPIEKT
ISKTKGRPKAPQVYTIPPPKEQMAKDKVSLTCMITDFFPEDITVEWQWNGQPAENYKNT
QPIMNTNGSYFVYSKLNVQKSNWEAGNTFTCSVLHEGLHNHHTEKSLSHSPGK
SEQ ID NO: 154: KD069 (variable region 1-146-mouse hinge-mouse constant region-IMGT
CDRH1, CDRH2 and CDRH3 in bold and underlined)
DVQLVESGGGSVQAGGSLRLSCAASGSTDSIEYMTWFRQAPGKAREGVAALYTHTGN
TYYTDSVKGRFTISQDKAKNMAYLRMDSVKSEDTAIYTCGATRKYVPVRFALDOSSY
DYWGQGTQVTVSSVPRDCGCKPCICTVPEVSSVFIFPPKPKDVLTITLTPKVTCVVVDISK
DDPEVQFSWFVDDVEVHTAQTQPREEQFNSTFRSVSELPIMHQDWLNGKEFKCRVNSA
AFPAPIEKTISKTKGRPKAPQVYTIPPPKEQMAKDKVSLTCMITDFFPEDITVEWQWNGQ
PAENYKNTQPIMNTNGSYFVYSKLNVQKSNWEAGNTFTCSVLHEGLHNHHTEKSLSHS
PGK
SEQ ID NO: 163 (linker)
APAPAPAPAPAPAPAPAPAPKA
SEQ ID NO: 164 (amino acid sequence 1 to 274 of human TROP2)
MARGPGLAPPPLRLPLLLLVLAAVTGHTAAQDNCTCPTNKMTVCSPDGPGGRCQCRAL
GSGMAVDCSTLTSKCLLLKARMSAPKNARTLVRPSEHALVDNDGLYDPDCDPEGRFKA
RQCNQTSVCWCVNSVGVRRTDKGDLSLRCDELVRTHHILIDLRHRPTAGAFNHSDLDA
ELRRLFRERYRLHPKFVAAVHYEQPTIQIELRQNTSQKAAGDVDIGDAAYYFERDIKGES
LFQGRGGLDLRVRGEPLQVERTLIYYLDEIPPKFSMKRLT

TABLE 22
Exemplary nucleotide sequence encoding the heavy chains of KD001-KD005

| Anti-TROP2 | Nucleotide Sequence |
|---|---|
| KD001 | CAGGTGCAATTGCAAGAGTCTGGCGGCGGAATGGTGCACCCTG |
| Heavy chain | GTGGATCTCTGAGACTGTCTTGTGCCGCCTCCGGCTTCACCTTC |
| SEQ ID NO: 165 | TCTGGCTCTGATATGTCCTGGGTCCGACAGGCTCCTGGCAAGG |
| | GACCTGAATGGGTGTCCTACATCACCTCCGGCGGCACCACCTA |
| | CTATCCCGACTCTGTGAAGGGCAGATTCACCATCTCTCGGGAC |
| | AACGCCAAGAACACCCTGTACCTGCAGATGAACTCTCTGACCC |
| | CTGACGACACAGCCCTGTACAGATGTGCCAAGGCCAGACTGAC |
| | CGACAGCCACTACGTGGAAGATGCTTGGGGCCAGGGAACACA |
| | GGTCACCGTGTCTAGTGAACCCAAGTCCTGCGACAAGACCCAC |
| | ACCTGTCCTCCATGTCCTGCTCCAGAACTGCTCGGCGGACCTTC |
| | CGTGTTCCTGTTTCCTCCAAAGCCTAAGGACACCCTGATGATCT |
| | CTCGGACCCCTGAAGTGACCTGCGTGGTGGTGGATGTGTCTCA |
| | CGAGGATCCCGAAGTGAAGTTCAATTGGTACGTGGACGGCGTG |
| | GAAGTGCACAACGCCAAGACCAAGCCTAGAGAGGAACAGTAC |
| | AACTCCACCTACAGAGTGGTGTCCGTGCTGACCGTGCTGCACC |
| | AGGATTGGCTGAACGGCAAAGAGTACAAGTGCAAGGTGTCCA |
| | ACAAGGCCCTGCCTGCTCCTATCGAAAAGACCATCTCCAAGGC |
| | CAAGGGCCAGCCTAGGGAACCCCAGGTTTACACCCTGCCTCCA |
| | AGCCGGGAAGAGATGACCAAGAACCAGGTGTCCCTGACCTGCC |
| | TGGTCAAGGGCTTCTACCCTTCCGATATCGCCGTGGAATGGGA |
| | GAGCAATGGCCAGCCTGAGAACAACTACAAGACAACCCCTCCT |
| | GTGCTGGACTCCGACGGCTCATTCTTCCTGTACTCCAAGCTGAC |
| | AGTGGACAAGTCCAGATGGCAGCAGGGCAACGTGTTCTCCTGC |
| | TCCGTGATGCACGAGGCCCTGCACAATCACTACACCCAGAAGT |
| | CCCTGTCTCTGTCCCCTGGCAAA |
| KD002 | CAGGTGCAATTGCAAGAGTCTGGCGGCGGAATGGTGCAAGTCG |
| Heavy chain | GCGGATCTCTGAGACTGTCTTGTGCCGCCTCTGGCTTCCCTTTC |
| SEQ ID NO: 166 | TCCTCCGCCGATATGTCCTGGGTCCGACAGGCTCCTGGAAAGG |
| | GACCTGAGTGGGTGTCCTACATCAACGCCGACGGCTCCAAGAC |
| | CTACTATCCCGACTCTGTGAAGGGCAGATTCACCATCTCTCGGG |
| | ACAACGCCAAGAACACCCTGTACCTGCTGATGAACAACCTGAA |
| | GCCTGAGGACACAGCCCTGTACAGATGCGCCAGAGCCAAGCTG |
| | ACCGACACACACTACGTGGAAGATTACTGGGGCCAGGGCACAC |
| | AGGTCACCGTGTCTAGTGAACCCAAGTCCTGCGACAAGACCCA |
| | CACCTGTCCTCCATGTCCTGCTCCAGAACTGCTCGGCGGACCTT |
| | CCGTGTTCCTGTTTCCTCCAAAGCCTAAGGACACCCTGATGATC |
| | TCTCGGACCCCTGAAGTGACCTGCGTGGTGGTGGATGTGTCTC |
| | ACGAGGATCCCGAAGTGAAGTTCAATTGGTACGTGGACGGCGT |
| | GGAAGTGCACAACGCCAAGACCAAGCCTAGAGAGGAACAGTA |
| | CAACTCCACCTACAGAGTGGTGTCCGTGCTGACCGTGCTGCAC |
| | CAGGATTGGCTGAACGGCAAAGAGTACAAGTGCAAGGTGTCC |
| | AACAAGGCCCTGCCTGCTCCTATCGAAAAGACCATCTCCAAGG |
| | CCAAGGGCCAGCCTAGGGAACCCCAGGTTTACACCCTGCCTCC |
| | AAGCCGGGAAGAGATGACCAAGAACCAGGTGTCCCTGACCTG |
| | CCTGGTCAAGGGCTTCTACCCTTCCGATATCGCCGTGGAATGG |
| | GAGAGCAATGGCCAGCCTGAGAACAACTACAAGACAACCCCT |
| | CCTGTGCTGGACTCCGACGGCTCATTCTTCCTGTACTCCAAGCT |
| | GACAGTGGACAAGTCCAGATGGCAGCAGGGCAACGTGTTCTCC |
| | TGCTCCGTGATGCACGAGGCCCTGCACAATCACTACACCCAGA |
| | AGTCCCTGTCTCTGTCCCCTGGCAAA |
| KD003 | CAGGTGCAATTGCAAGAGTCTGGCGGCGGAATGGTGCAGCCTG |
| Heavy chain | GTGGATCTCTGAGACTGTCTTGTGCCGCCTCCGGCTTCACCTTC |
| SEQ ID NO: 167 | TCCAACTACGACATGTCCTGGGTCCGACAGGCTCCTGGAAAGG |
| | GACCTGAGTGGGTGTCCTACATCAACGCTGGCGGCTCCAACAC |
| | CGACTATCCCGACTCTGTGAAGGGCAGATTCACCATCTCTCGG |
| | GACAACGCCAAGAACATGCTGTACCTGCTGATGAACAACCTGA |
| | AGCCTGAGGACACAGCCCTGTACAGATGCGCCAGAGCCAAGCT |
| | GACCGACACACACTACGTGGAAGATTACTGGGGCCAGGGCAC |
| | ACAGGTCACCGTGTCTAGTGAACCCAAGTCCTGCGACAAGACC |
| | CACACCTGTCCTCCATGTCCTGCTCCAGAACTGCTCGGCGGACC |
| | TTCCGTGTTCCTGTTTCCTCCAAAGCCTAAGGACACCCTGATGA |
| | TCTCTCGGACCCCTGAAGTGACCTGCGTGGTGGTGGATGTGTCT |
| | CACGAGGATCCCGAAGTGAAGTTCAATTGGTACGTGGACGGCG |
| | TGGAAGTGCACAACGCCAAGACCAAGCCTAGAGAGGAACAGT |
| | ACAACTCCACCTACAGAGTGGTGTCCGTGCTGACCGTGCTGCA |
| | CCAGGATTGGCTGAACGGCAAAGAGTACAAGTGCAAGGTGTCC |
| | AACAAGGCCCTGCCTGCTCCTATCGAAAAGACCATCTCCAAGG |
| | CCAAGGGCCAGCCTAGGGAACCCCAGGTTTACACCCTGCCTCC |
| | AAGCCGGGAAGAGATGACCAAGAACCAGGTGTCCCTGACCTG |
| | CCTGGTCAAGGGCTTCTACCCTTCCGATATCGCCGTGGAATGG |
| | GAGAGCAATGGCCAGCCTGAGAACAACTACAAGACAACCCCT |
| | CCTGTGCTGGACTCCGACGGCTCATTCTTCCTGTACTCCAAGCT |
| | GACAGTGGACAAGTCCAGATGGCAGCAGGGCAACGTGTTCTCC |
| | TGCTCCGTGATGCACGAGGCCCTGCACAATCACTACACCCAGA |
| | AGTCCCTGTCTCTGTCCCCTGGCAAA |
| KD004 | CAGGTGCAATTGCAAGAGTCTGGCGGCGGACTGGTGCAGCCTG |
| Heavy chain | GAAGATCTCTGAGACTGTCTTGTGCCGCCTCCGGCTTCACCTTC |
| SEQ ID NO: 168 | TCTGGCTCTGATATGTCCTGGGTCCGACAGGCTCCTGGCAAGG |
| | GACCTGAATGGGTGTCCTACATCACCGGCTCCGGCACCACCTA |
| | CTATCCCGACTCTGTGAAGGGCAGATTCACCACCTCCAGAGAC |
| | AACGCCAAGAACATGCTGTACCTGCAGATGAACTCCCTGAAGC |
| | CTGACGACACAGCCCTGTACAGATGCGCCAAGGCCAGACTGAC |
| | CGACAGCCACTACGTGGAAGATGCTTGGGGCCAGGGAACACA |
| | GGTCACCGTGTCTAGTGAACCCAAGTCCTGCGACAAGACCCAC |
| | ACCTGTCCTCCATGTCCTGCTCCAGAACTGCTCGGCGGACCTTC |
| | CGTGTTCCTGTTTCCTCCAAAGCCTAAGGACACCCTGATGATCT |
| | CTCGGACCCCTGAAGTGACCTGCGTGGTGGTGGATGTGTCTCA |
| | CGAGGATCCCGAAGTGAAGTTCAATTGGTACGTGGACGGCGTG |
| | GAAGTGCACAACGCCAAGACCAAGCCTAGAGAGGAACAGTAC |
| | AACTCCACCTACAGAGTGGTGTCCGTGCTGACCGTGCTGCACC |
| | AGGATTGGCTGAACGGCAAAGAGTACAAGTGCAAGGTGTCCA |
| | ACAAGGCCCTGCCTGCTCCTATCGAAAAGACCATCTCCAAGGC |
| | CAAGGGCCAGCCTAGGGAACCCCAGGTTTACACCCTGCCTCCA |
| | AGCCGGGAAGAGATGACCAAGAACCAGGTGTCCCTGACCTGCC |
| | TGGTCAAGGGCTTCTACCCTTCCGATATCGCCGTGGAATGGGA |
| | GAGCAATGGCCAGCCTGAGAACAACTACAAGACAACCCCTCCT |
| | GTGCTGGACTCCGACGGCTCATTCTTCCTGTACTCCAAGCTGAC |
| | AGTGGACAAGTCCAGATGGCAGCAGGGCAACGTGTTCTCCTGC |
| | TCCGTGATGCACGAGGCCCTGCACAATCACTACACCCAGAAGT |
| | CCCTGTCTCTGTCCCCTGGCAAA |
| KD005 | CAGGTGCAATTGCAAGAGTCTGGCGGCGGACTGGTGGAAGCTG |
| Heavy chain | GTGGATCTCTGAGACTGTCTTGTGCCGCCTCCGGCTTCACCTTC |
| SEQ ID NO: 169 | TCTGGCTCTGATATGTCCTGGGTCCGACAGGCTCCTGGCAAGG |
| | GACCTGAATGGGTGTCCTACATCACCTCCGGCGGCACCACCTA |
| | CTATCCCGACTCTGTGAAGGGCAGATTCACCATCTCTCGGGAC |
| | AACGCCAAGAACACCCTGTACCTGCAGATGAACTCCCTGAAGC |
| | CTGACGACACAGCCCTGTACAGATGCGCCAAGTCTCGGCTGAC |
| | CGACAGCCACTACGTGGAAGATGCTTGGGGCCAGGGAACACA |
| | GGTCACCGTGTCTAGTGAACCCAAGTCCTGCGACAAGACCCAC |
| | ACCTGTCCTCCATGTCCTGCTCCAGAACTGCTCGGCGGACCTTC |
| | CGTGTTCCTGTTTCCTCCAAAGCCTAAGGACACCCTGATGATCT |
| | CTCGGACCCCTGAAGTGACCTGCGTGGTGGTGGATGTGTCTCA |
| | CGAGGATCCCGAAGTGAAGTTCAATTGGTACGTGGACGGCGTG |
| | GAAGTGCACAACGCCAAGACCAAGCCTAGAGAGGAACAGTAC |
| | AACTCCACCTACAGAGTGGTGTCCGTGCTGACCGTGCTGCACC |
| | AGGATTGGCTGAACGGCAAAGAGTACAAGTGCAAGGTGTCCA |
| | ACAAGGCCCTGCCTGCTCCTATCGAAAAGACCATCTCCAAGGC |
| | CAAGGGCCAGCCTAGGGAACCCCAGGTTTACACCCTGCCTCCA |
| | AGCCGGGAAGAGATGACCAAGAACCAGGTGTCCCTGACCTGCC |
| | TGGTCAAGGGCTTCTACCCTTCCGATATCGCCGTGGAATGGGA |
| | GAGCAATGGCCAGCCTGAGAACAACTACAAGACAACCCCTCCT |
| | GTGCTGGACTCCGACGGCTCATTCTTCCTGTACTCCAAGCTGAC |
| | AGTGGACAAGTCCAGATGGCAGCAGGGCAACGTGTTCTCCTGC |
| | TCCGTGATGCACGAGGCCCTGCACAATCACTACACCCAGAAGT |
| | CCCTGTCTCTGTCCCCTGGCAAA |

SEQ ID NO: 176 (CD47 Antigen)
MWPLVAALLLGSACCGSAQLLENKTKSVEFTFCNDTVVIPCFVTNMEAQ
NTTEVYVKWKFKGRDIYTFDGALNKSTVPTDFSSAKIEVSQLLKGDASL
KMDKSDAVSHTGNYTCEVTELTREGETIIELKYRVVSWFSP
SEQ ID NO: 212: Dimerisation domain based on IgG1
constant region (hinge portion optional or
substitutable by other linkers)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 506: Dimerisation domain based on IgG1
constant region (hinge portion optional or
substitutable by other linkers)
EPKSSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 214 Dimerisation domain based on IgG4
constant region (hinge portion optional or
substitutable by other linkers)
ESKYGPPCPPCPAPEAAGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

TABLE 23
Exemplary humanized heavy chain variable region backbones

| | SEQUENCE |
|---|---|
| SEQ ID NO: | IMGT CDRs identified as [CDRH1], [CDRH2] and [CDRH3] |
| SEQ ID NO: 248 | QVQLQESGGGMVQVGGSLRLSCAAS[CDRH1]MSWVRQAPGKGP |
| | EWVSY[CDRH2]YYPDSVKGRFTISRDNAKNTLYLLMNNLKPEDT |
| | ALYRC[CDRH3]WGQGTQVTVSS |
| SEQ ID NO: 249 | EVOLVESGGGLVQPGGSLRLSCAAS[CDRH1]MHWVRQAPGKGL |
| | VWVSR[CDRH2]SYADTVKGRFTISRDNAKNTLYLQMNSLRAEDT |
| | AVYYC[CDRH3]WGQGTLVTVSS |
| SEQ ID NO: 250 | EVOLVESGGGLVQPGGSLRLSCAAS[CDRH1]MSWVRQAPGKGLE |
| | WVSY[CDRH2]SYADTVKGRFTISRDNAKNTLYLQMNSLRAEDTA |
| | VYYC[CDRH3]WGQGTLVTVSS |
| SEQ ID NO: 251 | EVOLVESGGGLVQPGGSLRLSCAAS[CDRH1]MSWVRQAPGKGPE |
| | WVSY[CDRH2]YYADTVKGRFTISRDNAKNTLYLQMNSLRAEDT |
| | AVYYC[CDRH3]WGQGTQVTVSS |
| SEQ ID NO: 252 | QVQLVESGGGLVQPGGSLRLSCAAS[CDRH1]MSWVRQAPGKGPE |
| | WVSY[CDRH2]YYPDTVKGRFTISRDNAKNTLYLQMNSLRAEDTA |
| | VYYC[CDRH3]WGQGTQVTVSS |
| SEQ ID NO: 253 | QVQLVESGGGLVQPGGSLRLSCAAS[CDRH1]MSWVRQAPGKGPE |
| | WVSY[CDRH2]YYPDTVKGRFTISRDNAKNTLYLQMNSLRAEDTA |
| | VYRC[CDRH3]WGQGTQVTVSS |
| SEQ ID NO: 254 | QVQLVESGGGLVQVGGSLRLSCAAS[CDRH1]MSWVRQAPGKGP |
| | EWVSY[CDRH2]YYPDTVKGRFTISRDNAKNTLYLQMNSLKPEDT |
| | AVYRC[CDRH3]WGQGTQVTVSS |
| SEQ ID NO: 255 | QVQLQESGGGMVQVGGSLRLSCAAS[CDRH1]MSWVRQAPGKGP |
| | EWVSY[CDRH2]YYPDTVKGRFTISRDNAKNTLYLQMNSLKPEDT |
| | AVYRC[CDRH3]WGQGTQVTVSS |
| SEQ ID NO: 256 | QVQLQESGGGMVQVGGSLRLSCAAS[CDRH1]MSWVRQAPGKGP |
| | EWVSY[CDRH2]YYPDSVKGRFTISRDNAKNTLYLQMNSLKPEDT |
| | AVYRC[CDRH3]WGQGTQVTVSS |
| SEQ ID NO: 257 | EVQLLESGGGLVQPGGSLRLSCAAS[CDRH1]MSWVRQAPGKGLE |
| | WVSV[CDRH2]YYADTVKGRFTISRDNSKNTLYLQMNSLRAEDTA |
| | VYYC[CDRH3]WGQGTMVTVSS |
| SEQ ID NO: 258 | QVQLQESGGGLVQPGGSLRLSCAAS[CDRH1] |
| | MSWVRQAPGKGPEWVSY[CDRH2]YYPDTVKGRFTISRDNSKNTL |
| | YLQMNNLRAEDTALYRC[CDRH3]WGQGTMVTVSS |
| SEQ ID NO: 259 | QVQLQESGGGLVQPGGSLRLSCAAS[CDRH1] |
| | MSWVRQAPGKGLEWVSY[CDRH2]YYADTVKGRFTISRDNSKNT |
| | LYLQMNSLRAEDTAVYRC[CDRH3]WGQGTMVTVSS |
| SEQ ID NO: 260 | EVQLQESGGGLVQPGGSLRLSCAAS[CDRH1]MSWVRQAPGKGLE |
| | WVSY[CDRH2]YYADTVKGRFTISRDNSKNTLYLQMNSLRAEDTA |
| | VYRC[CDRH3]WGQGTMVTVSS |
| SEQ ID NO: 261 | QVQLLESGGGLVQPGGSLRLSCAAS[CDRH1]MSWVRQAPGKGLE |
| | WVSY[CDRH2]YYADTVKGRFTISRDNSKNTLYLQMNSLRAEDTA |
| | VYRC[CDRH3]WGQGTMVTVSS |
| SEQ ID NO: 262 | QVQLQESGGGLVQPGGSLRLSCAAS[CDRH1]MSWVRQAPGKGL |
| | EWVSV[CDRH2]YYADTVKGRFTISRDNSKNTLYLQMNSLRAEDT |
| | AVYRC[CDRH3]WGQGTMVTVSS |
| SEQ ID NO: 263 | QVQLQESGGGLVQPGGSLRLSCAAS[CDRH1]MSWVRQAPGKGL |
| | EWVSY[CDRH2]YYADTVKGRFTISRDNSKNTLYLQMNSLRAEDT |
| | AVYYC[CDRH3] WGQGTMVTVSS |
| SEQ ID NO: 264 | QVQLQESGGGLVQPGGSLRLSCAAS[CDRH1]MSWVRQAPGKGPE |
| | WVSY[CDRH2]YYADTVKGRFTISRDNSKNTLYLQMNSLRAEDTA |
| | VYRC[CDRH3]WGQGTMVTVSS |
| SEQ ID NO: 265 | QVQLQESGGGLVQPGGSLRLSCAAS[CDRH1]MSWVRQAPGKGL |
| | EWVSY[CDRH2]YYPDTVKGRFTISRDNSKNTLYLQMNSLRAEDT |
| | AVYRC[CDRH3]WGQGTMVTVSS |
| SEQ ID NO: 266 | QVQLQESGGGLVQPGGSLRLSCAAS[CDRH1]MSWVRQAPGKGL |
| | EWVSY[CDRH2]YYADTVKGRFTISRDNSKNTLYLQMNNLRAEDT |
| | AVYRC[CDRH3]WGQGTMVTVSS |
| SEQ ID NO: 267 | QVQLQESGGGLVQPGGSLRLSCAAS[CDRH1]MSWVRQAPGKGL |
| | EWVSY[CDRH2]YYADTVKGRFTISRDNSKNTLYLQMNSLRAEDT |
| | ALYRC[CDRH3]WGQGTMVTVSS |
| SEQ ID NO: 268 | QVQLQESGGGMVQPGGSLRLSCAAS[CDRH1]MSWVRQAPGKGL |
| | EWVSY[CDRH2]YYADTVKGRFTISRDNSKNTLYLQMNSLRAEDT |
| | AVYRC[CDRH3]WGQGTQVTVSS |
| SEQ ID NO: 269 | QVQLQESGGGLVQPGGSLRLSCAAS[CDRH1]MSWVRQAPGKQR |
| | EWVSY[CDRH2]YYADTVKGRFTISRDNSKNTLYLQMNSLRAEDT |
| | AVYRC[CDRH3]WGQGTMVTVSS |
| SEQ ID NO: 270 | QVQLQESGGGLVQPGGSLRLSCAAS[CDRH1]MSWVRQAPGKGL |
| | EWVSY[CDRH2]YYADTVKGRFTISRDNSKNTLYLQMNSLRAEDT |
| | AVYRC[CDRH3] WGQGTMVTVSS |
| SEQ ID NO: 271 | QVQLQESGGGLVQPGGSLRLSCAAS[CDRH1]MSWVRQAPGKGL |
| | EWVSY[CDRH2]YYADTVKGRFTISRDNSKNTLYLQMNSLRAEDT |
| | AVYRC[CDRH3]WGQGTMVTVSS |

TABLE 24
Exemplary humanized heavy chain variable region backbones

| | SEQUENCE |
|---|---|
| SEQ ID NO: | IMGT CDRs |
| SEQ ID NO: 272 | QVQLQESGGGLVEAGGSLRLSCAAS[CDRH1]MSWVRQAPGKGP |
| | EWVSY[CDRH2]YYPDSVKGRFTISRDNAKNTLYLQMNSLKPDDTALY |
| | RC[CDRH3]WGQGTQVTVSS |
| SEQ ID NO: 273 | EVQLLESGGGLVQPGGSLRLSCAAS[CDRH1]MSWVRQAPGKGLE |
| | WVSA[CDRH2]YYADTVKGRFTISRDNSKNTLYLQMNSLRAEDTAVYY |
| | C[CDRH3]WGQGTLVTVSS |
| SEQ ID NO: 274 | EVQLLESGGGLVQPGGSLRLSCAAS[CDRH1]MSWVRQAPGKGLE |
| | WVSY[CDRH2]YYADTVKGRFTISRDNSKNTLYLQMNSLRAEDTAVYY |
| | C[CDRH3]WGQGTLVTVSS |
| SEQ ID NO: 275 | EVQLLESGGGLVQPGGSLRLSCAAS[CDRH1]MSWVRQAPGKGLE |
| | WVSY[CDRH2]YYADTVKGRFTISRDNSKNTLYLQMNSLRAEDTAVYR |
| | C[CDRH3]WGQGTLVTVSS |
| SEQ ID NO: 276 | QVQLLESGGGLVQPGGSLRLSCAAS[CDRH1]MSWVRQAPGKGPE |
| | WVSY[CDRH2]YYADTVKGRFTISRDNSKNTLYLQMNSLRAEDTAVYY |
| | C[CDRH3]WGQGTLVTVSS |
| SEQ ID NO: 277 | QVQLLESGGGLVQPGGSLRLSCAAS[CDRH1]MSWVRQAPGKGPE |
| | WVSY[CDRH2]YYADTVKGRFTISRDNSKNTLYLQMNSLRAEDTAVYR |
| | C[CDRH3]WGQGTLVTVSS |
| SEQ ID NO: 278 | QVQLQESGGGLVQPGGSLRLSCAAS[CDRH1]MSWVRQAPGKGPE |
| | WVSY[CDRH2]YYPDTVKGRFTISRDNSKNTLYLQMNSLRAEDTAVYY |
| | C[CDRH3]WGQGTQVTVSS |
| SEQ ID NO: 279 | QVQLQESGGGLVQPGGSLRLSCAAS[CDRH1]MSWVRQAPGKGPE |
| | WVSY[CDRH2]YYPDTVKGRFTISRDNSKNTLYLQMNSLRAEDTAVYR |
| | C[CDRH3]WGQGTQVTVSS |
| SEQ ID NO: 280 | QVQLQESGGGLVQPGGSLRLSCAAS[CDRH1]MSWVRQAPGKGPE |
| | WVSY[CDRH2]YYPDTVKGRFTISRDNSKNTLYLQMNSLKPDDTAVYR |
| | C[CDRH3]WGQGTQVTVSS |
| SEQ ID NO: 281 | QVQLQESGGGLVQPGGSLRLSCAAS[CDRH1]MSWVRQAPGKGPE |
| | WVSY[CDRH2]YYPDTVKGRFTISRDNAKNTLYLQMNSLKPDDTALYR |
| | C[CDRH3]WGQGTQVTVSS |
| SEQ ID NO: 282 | EVOLVESGGGLIQPGGSLRLSCAAS[CDRH1]MSWVRQAPGKGLE |
| | WVSV[CDRH2]YYADTVKGRFTISRDNSKNTLYLQMNSLRAEDTAVYY |
| | C[CDRH3]WGQGTLVTVSS |
| SEQ ID NO: 283 | QVQLQESGGGLIQPGGSLRLSCAAS[CDRH1]MSWVRQAPGKGPE |
| | WVSY[CDRH2]YYPDTVKGRFTISRDNSKNTLYLQMNSLRADDTALYR |
| | C[CDRH3]WGQGTLVTVSS |
| SEQ ID NO: 284 | QVQLQESGGGLIQPGGSLRLSCAAS[CDRH1]MSWVRQAPGKGLE |
| | WVSY[CDRH2]YYADTVKGRFTISRDNSKNTLYLQMNSLRAEDTAVYR |
| | C[CDRH3]WGQGTLVTVSS |
| SEQ ID NO: 285 | EVQLQESGGGLIQPGGSLRLSCAAS[CDRH1]MSWVRQAPGKGLE |
| | WVSY[CDRH2]YYADTVKGRFTISRDNSKNTLYLQMNSLRAEDTAVYR |
| | C[CDRH3]WGQGTLVTVSS |
| SEQ ID NO: 286 | QVQLVESGGGLIQPGGSLRLSCAAS[CDRH1]MSWVRQAPGKGLE |
| | WVSY[CDRH2]YYADTVKGRFTISRDNSKNTLYLQMNSLRAEDTAVYR |
| | C[CDRH3]WGQGTLVTVSS |
| SEQ ID NO: 287 | QVQLQESGGGLIQPGGSLRLSCAAS[CDRH1]MSWVRQAPGKGLE |
| | WVSV[CDRH2]YYADTVKGRFTISRDNSKNTLYLQMNSLRAEDTAVYR |
| | C[CDRH3]WGQGTLVTVSS |
| SEQ ID NO: 288 | QVQLQESGGGLIQPGGSLRLSCAAS[CDRH1]MSWVRQAPGKGLE |
| | WVSY[CDRH2]YYADTVKGRFTISRDNSKNTLYLQMNSLRAEDTAVYY |
| | C[CDRH3]WGQGTLVTVSS |
| SEQ ID NO: 289 | QVQLQESGGGLIQPGGSLRLSCAAS[CDRH1]MSWVRQAPGKGPE |
| | WVSY[CDRH2]YYADTVKGRFTISRDNSKNTLYLQMNSLRAEDTAVYR |
| | C[CDRH3]WGQGTLVTVSS |
| SEQ ID NO: 290 | QVQLQESGGGLIQPGGSLRLSCAAS[CDRH1]MSWVRQAPGKGLE |
| | WVSY[CDRH2]YYPDTVKGRFTISRDNSKNTLYLQMNSLRAEDTAVYR |
| | C[CDRH3]WGQGTLVTVSS |
| SEQ ID NO: 291 | QVQLQESGGGLIQPGGSLRLSCAAS[CDRH1]MSWVRQAPGKGLE |
| | WVSY[CDRH2]YYADTVKGRFTISRDNSKNTLYLQMNSLRADDTAVYR |
| | C[CDRH3]WGQGTLVTVSS |
| SEQ ID NO: 292 | QVQLQESGGGLIQPGGSLRLSCAAS[CDRH1]MSWVRQAPGKGLE |
| | WVSY[CDRH2]YYADTVKGRFTISRDNSKNTLYLQMNSLRAEDTALYR |
| | C[CDRH3]WGQGTLVTVSS |
| SEQ ID NO: 293 | QVQLQESGGGLIEPGGSLRLSCAAS[CDRH1]MSWVRQAPGKGLE |
| | WVSY[CDRH2]YYADTVKGRFTISRDNSKNTLYLQMNSLRAEDTAVYR |
| | C[CDRH3]WGQGTQVTVSS |
| SEQ ID NO: 294 | QVQLQESGGGLIQPGGSLRLSCAAS[CDRH1]MSWYRQAPGKGRE |
| | WVSY[CDRH2]YYADTVKGRFTISRDNSKNTLYLQMNSLRAEDTAVYR |
| | C[CDRH3]WGQGTLVTVSS |
| SEQ ID NO: 474 | EVQLLESGGGMVQPGGSLRLSCAAS[CDRH1]MSWVRQAPGKGL |
| | EWVSY[CDRH2]YYADTVKGRFTISRDNSKNTLYLQMNSLRAEDTAVY |
| | RC[CDRH3]WGQGTLVTVSS |
| SEQ ID NO: 475 | QVQLQESGGGMIQPGGSLRLSCAAS[CDRH1]MSWVRQAPGKGPE |
| | WVSY[CDRH2]YYPDTVKGRFTISRDNSKNTLYLQMNSLRADDTALYR |
| | C[CDRH3]WGQGTLVTVSS |
| SEQ ID NO: 476 | QVQLQESGGGLVHPGGSLRLSCAAS[CDRH1]MSWVRQAPGKGPE |
| | WVSY[CDRH2]YYPDTVKGRFTISRDNAKNTLYLQMNSLTPEDTALYR |
| | C[CDRH3]WGQGTQVTVSS |

1. A binding agent comprising one or more antigen binding domains, wherein at least one of the one or more antigen binding domains binds to trophoblast cell surface antigen-2 (TROP2) and comprises:
a. a heavy chain complementarity determining region 1 (CDRH1) having the amino acid sequence set forth in SEQ ID NO:500, a heavy chain complementarity determining region 2 (CDRH2) having the amino acid sequence set forth in SEQ ID NO:489 and a heavy chain complementarity determining region 3 (CDRH3) having the amino acid sequence set forth in SEQ ID NO:501,
b. a CDRH1 having the amino acid sequence set forth in SEQ ID NO:485, a CDRH2 having the amino acid sequence set forth in SEQ ID NO:498 or in SEQ ID NO:486 and a CDRH3 having the amino acid sequence set forth in SEQ ID NO:499,
c. A CDRH1 having the amino acid sequence set forth in SEQ ID NO:225, a CDRH2 having the amino acid sequence set forth in SEQ ID NO:226, SEQ ID NO:234, SEQ ID NO:242, SEQ ID NO:320, SEQ ID NO: 392 and a CDRH3 having the amino acid sequence set forth in SEQ ID NO:227-er;
d. A CDRH1 having the amino acid sequence set forth in SEQ ID NO:228, a CDRH2 having the amino acid sequence set forth in SEQ ID NO:229, SEQ ID NO:237, SEQ ID NO:245 or SEQ ID NO:323 or SEQ ID NO:395 and a CDRH3 having the amino acid sequence set forth in SEQ ID NO:230;
e. a CDRH1 having the amino acid sequence set forth in SEQ ID NO:488, a CDRH2 having the amino acid sequence set forth in SEQ ID NO:489 and a CDRH3 having the amino acid sequence set forth in SEQ ID NO:490,
f. a CDRH1 having the amino acid sequence set forth in SEQ ID NO:485, a CDRH2 having the amino acid sequence set forth in SEQ ID NO:486, SEQ ID NO:492 or SEQ ID NO:495 and a CDRH3 having the amino acid sequence set forth in SEQ ID NO:487,
g. a CDRH1 having the amino acid sequence as set forth in SEQ ID NO:305, a CDRH2 having the amino acid sequence as set forth in SEQ ID NO:306 and a CDRH3 having the amino acid sequence as set forth in SEQ ID NO:307;
h. a CDRH1 having the amino acid sequence as set forth in SEQ ID NO:308, a CDRH2 having the amino acid sequence as set forth in SEQ ID NO:309 and a CDRH3 having the amino acid sequence as set forth in SEQ ID NO:310;
i. a CDRH1 having the amino acid sequence as set forth in SEQ ID NO:312, a CDRH2 having the amino acid sequence as set forth in SEQ ID NO:313 and a CDRH3 having the amino acid sequence as set forth in SEQ ID NO:314;
j. a CDRH1 having the amino acid sequence as set forth in SEQ ID NO:315, a CDRH2 having the amino acid sequence as set forth in SEQ ID NO:316 and a CDRH3 having the amino acid sequence as set forth in SEQ ID NO:317;
k. a CDRH1 having the amino acid sequence as set forth in SEQ ID NO:326, a CDRH2 having the amino acid sequence as set forth in SEQ ID NO:327 and a CDRH3 having the amino acid sequence as set forth in SEQ ID NO:328;
l. a CDRH1 having the amino acid sequence as set forth in SEQ ID NO:329, a CDRH2 having the amino acid sequence as set forth in SEQ ID NO:330 and a CDRH3 having the amino acid sequence as set forth in SEQ ID NO:331;
m. a CDRH1 having the amino acid sequence as set forth in SEQ ID NO:333, a CDRH2 having the amino acid sequence as set forth in SEQ ID NO:334 and a CDRH3 having the amino acid sequence as set forth in SEQ ID NO:335;
n. a CDRH1 having the amino acid sequence as set forth in SEQ ID NO:336, a CDRH2 having the amino acid sequence as set forth in SEQ ID NO:337 and a CDRH3 having the amino acid sequence as set forth in SEQ ID NO:338;
o. a CDRH1 having the amino acid sequence as set forth in SEQ ID NO:340, a CDRH2 having the amino acid sequence as set forth in SEQ ID NO:341 and a CDRH3 having the amino acid sequence as set forth in SEQ ID NO:342;
p. a CDRH1 having the amino acid sequence as set forth in SEQ ID NO:343, a CDRH2 having the amino acid sequence as set forth in SEQ ID NO:344 and a CDRH3 having the amino acid sequence as set forth in SEQ ID NO:345;
q. a CDRH1 having the amino acid sequence as set forth in SEQ ID NO:347, a CDRH2 having the amino acid sequence as set forth in SEQ ID NO:348 and a CDRH3 having the amino acid sequence as set forth in SEQ ID NO:349;
r. a CDRH1 having the amino acid sequence as set forth in SEQ ID NO:350, a CDRH2 having the amino acid sequence as set forth in SEQ ID NO:351 and a CDRH3 having the amino acid sequence as set forth in SEQ ID NO:352;
s. a CDRH1 having the amino acid sequence as set forth in SEQ ID NO:399, a CDRH2 having the amino acid sequence as set forth in SEQ ID NO:400 and a CDRH3 having the amino acid sequence as set forth in SEQ ID NO:401;
t. a CDRH1 having the amino acid sequence as set forth in SEQ ID NO:402, a CDRH2 having the amino acid sequence as set forth in SEQ ID NO:403 and a CDRH3 having the amino acid sequence as set forth in SEQ ID NO:404;
u. a CDRH1 having the amino acid sequence as set forth in SEQ ID NO:406, a CDRH2 having the amino acid sequence as set forth in SEQ ID NO:407 and a CDRH3 having the amino acid sequence as set forth in SEQ ID NO:408;
v. a CDRH1 having the amino acid sequence as set forth in SEQ ID NO:409, a CDRH2 having the amino acid sequence as set forth in SEQ ID NO:410 and a CDRH3 having the amino acid sequence as set forth in SEQ ID NO:411;
w. a CDRH1 having the amino acid sequence as set forth in SEQ ID NO:413, a CDRH2 having the amino acid sequence as set forth in SEQ ID NO:414 and a CDRH3 having the amino acid sequence as set forth in SEQ ID NO:415;
x. a CDRH1 having the amino acid sequence as set forth in SEQ ID NO:416, a CDRH2 having the amino acid sequence as set forth in SEQ ID NO:417 and a CDRH3 having the amino acid sequence as set forth in SEQ ID NO:418;
y. a CDRH1 having the amino acid sequence as set forth in SEQ ID NO:420, a CDRH2 having the amino acid sequence as set forth in SEQ ID NO:421 and a CDRH3 having the amino acid sequence as set forth in SEQ ID NO:422;
z. a CDRH1 having the amino acid sequence as set forth in SEQ ID NO:423, a CDRH2 having the amino acid sequence as set forth in SEQ ID NO:424 and a CDRH3 having the amino acid sequence as set forth in SEQ ID NO:425;
aa. a CDRH1 having the amino acid sequence as set forth in SEQ ID NO:427, a CDRH2 having the amino acid sequence as set forth in SEQ ID NO:428 and a CDRH3 having the amino acid sequence as set forth in SEQ ID NO:429;
bb. a CDRH1 having the amino acid sequence as set forth in SEQ ID NO:430, a CDRH2 having the amino acid sequence as set forth in SEQ ID NO:431 and a CDRH3 having the amino acid sequence as set forth in SEQ ID NO:432;
cc. a CDRH1 having the amino acid sequence as set forth in SEQ ID NO:434, a CDRH2 having the amino acid sequence as set forth in SEQ ID NO:435 and a CDRH3 having the amino acid sequence as set forth in SEQ ID NO:436;
dd. a CDRH1 having the amino acid sequence as set forth in SEQ ID NO:437, a CDRH2 having the amino acid sequence as set forth in SEQ ID NO:438 and a CDRH3 having the amino acid sequence as set forth in SEQ ID NO:439;
ee. a CDRH1 having the amino acid sequence as set forth in SEQ ID NO:441, a CDRH2 having the amino acid sequence as set forth in SEQ ID NO:442 and a CDRH3 having the amino acid sequence as set forth in SEQ ID NO:443;
ff. a CDRH1 having the amino acid sequence as set forth in SEQ ID NO:444, a CDRH2 having the amino acid sequence as set forth in SEQ ID NO:445 and a CDRH3 having the amino acid sequence as set forth in SEQ ID NO:446;
gg. a CDRH1 having the amino acid sequence as set forth in SEQ ID NO:448, a CDRH2 having the amino acid sequence as set forth in SEQ ID NO:449 and a CDRH3 having the amino acid sequence as set forth in SEQ ID NO:450;
hh. a CDRH1 having the amino acid sequence as set forth in SEQ ID NO:451, a CDRH2 having the amino acid sequence as set forth in SEQ ID NO:452 and a CDRH3 having the amino acid sequence as set forth in SEQ ID NO:453;
ii. a CDRH1 having the amino acid sequence as set forth in SEQ ID NO:455, a CDRH2 having the amino acid sequence as set forth in SEQ ID NO:456 and a CDRH3 having the amino acid sequence as set forth in SEQ ID NO:457;
jj. a CDRH1 having the amino acid sequence as set forth in SEQ ID NO:458, a CDRH2 having the amino acid sequence as set forth in SEQ ID NO:459 and a CDRH3 having the amino acid sequence as set forth in SEQ ID NO:460;
kk. a CDRH1 having the amino acid sequence as set forth in SEQ ID NO:462, a CDRH2 having the amino acid sequence as set forth in SEQ ID NO:463 and a CDRH3 having the amino acid sequence as set forth in SEQ ID NO:464; or
ll. a CDRH1 having the amino acid sequence as set forth in SEQ ID NO:465, a CDRH2 having the amino acid sequence as set forth in SEQ ID NO:466 and a CDRH3 having the amino acid sequence as set forth in SEQ ID NO:467.

2.-9. (canceled)

10. The binding agent of claim 1, wherein the at least one of the one or more antigen binding domains comprises:
a. a CDRH1 having the amino acid sequence set forth in SEQ ID NO:500, a CDRH2 having the amino acid sequence set forth in SEQ ID NO:489 and a CDRH3 having the amino acid sequence set forth in SEQ ID NO:501 and an amino acid sequence at least 80% identical to the amino acid sequence set forth in any one of SEQ ID NO:503, SEQ ID NO:502 or SEQ ID NO: 504;
b. a CDRH1 having the amino acid sequence set forth in SEQ ID NO:488, a CDRH2 having the amino acid sequence set forth in SEQ ID NO:489 and a CDRH3 having the amino acid sequence set forth in SEQ ID NO:490 and an amino acid sequence at least 80% identical to the amino acid sequence set forth in any one of SEQ ID NO: 493, SEQ ID NO:496 or SEQ ID NO:491;
c. a CDRH1 having the amino acid sequence set forth in SEQ ID NO:308, a (CDRH2 having the amino acid sequence set forth in SEQ ID NO:309 and a CDRH3 having the amino acid sequence set forth in SEQ ID NO:310 and an amino acid sequence at least 80% identical to the amino acid sequence set forth in any one of SEQ ID NO:311 or SEQ ID NO:389; or
d. an amino acid sequence at least 80% identical to the amino acid sequence set forth in SEQ ID NO:373, SEQ ID NO:232, SEQ ID NO:231, SEQ ID NO:295, SEQ ID NO:296, SEQ ID NO:297, SEQ ID NO:372, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:239, SEQ ID NO:240, SEQ ID NO:383, SEQ ID NO:384, SEQ ID NO:385, SEQ ID NO:386, SEQ ID NO:387, SEQ ID NO:247, SEQ ID NO:388, SEQ ID NO:325, SEQ ID NO:390, SEQ ID NO:397, or SEQ ID NO:398.

11.-15. (canceled)

16. The binding agent of claim 1, wherein the at least one of the one or more antigen binding domains comprises humanized framework regions.

17. (canceled)

18. The binding agent of claim 1, wherein the at least one of the one or more antigen binding domains comprises:
a. a humanized heavy chain variable region backbone comprising the amino acid sequence set forth in SEQ ID NO:211, SEQ ID NO:477 or SEQ ID NO:478;
b. a humanized heavy chain variable region backbone comprising the amino acid sequence set forth in SEQ ID NO:213, SEQ ID NO:479 or SEQ ID NO:480;
c. a humanized heavy chain variable region backbone comprising the amino acid sequence set forth in SEQ ID NO: 248, SEQ ID NO:249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, 266, SEQ ID NO: 267, SEQ ID NO: 268, SEQ ID NO: 269, SEQ ID NO: 270 or SEQ ID NO: 271 or comprising from one to five amino acid substitutions, additions and/or deletions thereto; or
d. a humanized heavy chain variable region backbone comprising the amino acid sequence set forth in SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 474, SEQ ID NO: 475 and SEQ ID NO: 476 or comprising from one to five amino acid substitutions, additions and/or deletions thereto.

19.-21. (canceled)

22. The binding agent of claim 1, wherein the heavy chain variable region comprises a framework region 2 (FR2) comprising the sequence set forth in SEQ ID NO:116, SEQ ID NO:117, SEQ ID NO:118, SEQ ID NO:119, SEQ ID NO:120, SEQ ID NO:354, SEQ ID NO:355, SEQ ID NO:356, SEQ ID NO:360, SEQ ID NO:361 or SEQ ID NO:505.

23. A binding agent comprising one or more antigen binding domains, wherein at least one of the one or more antigen binding domains binds to trophoblast cell surface antigen-2 (TROP2) and comprises the amino acid sequence set forth in SEQ ID NO:502, SEQ ID NO:503, SEQ ID NO:504, SEQ ID NO:373, SEQ ID NO: 493, SEQ ID NO:221, SEQ ID NO:222, SEQ ID NO:223, SEQ ID NO:224, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:469, SEQ ID NO:470, SEQ ID NO:471, SEQ ID NO:472, SEQ ID NO:473, SEQ ID NO:304, SEQ ID NO:231, SEQ ID NO:232, SEQ ID NO:295, SEQ ID NO:296, SEQ ID NO:297, SEQ ID NO:372, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:239, SEQ ID NO:240, SEQ ID NO:383, SEQ ID NO:384, SEQ ID NO:385, SEQ ID NO:386, SEQ ID NO:387, SEQ ID NO:247, SEQ ID NO:388, SEQ ID NO:325, SEQ ID NO:390, SEQ ID NO:397, SEQ ID NO:398, SEQ ID NO:311, SEQ ID NO:389, SEQ ID NO:318, SEQ ID NO:332, SEQ ID NO:339, SEQ ID NO:346, SEQ ID NO:353, SEQ ID NO:405, SEQ ID NO:412, SEQ ID NO:419, SEQ ID NO:426, SEQ ID NO:433, SEQ ID NO:440, SEQ ID NO:447, SEQ ID NO:454, SEQ ID NO:461, SEQ ID NO:468, SEQ ID NO:491, or SEQ ID NO:496.

24. (canceled)

25. The binding agent of claim 1, wherein the at least one of the one or more antigen binding domains is a heavy chain variable region of a heavy chain only antibody or comprises a heavy chain variable region of heavy chain only antibody.

26. (canceled)

27. The binding agent of claim 25, wherein the one or mere heavy chain variable regions are from a camelid VH or VHH.

28. The binding agent of claim 1, wherein the binding agent comprises a) two antigen binding domains or more and/or is monospecific, or b) comprises two antigen binding domains or more and/or is bispecific or multispecific.

29.-31. (canceled)

32. The binding agent of claim 1, wherein the binding agent comprises one or more antigen binding domain that binds to CD47 and/or one or more antigen binding domain that binds to PD-1.

33. The binding agent of claim 1, wherein the binding agent comprises:
a. two polypeptide chains and wherein each polypeptide chain comprises in a N- to C-terminal fashion, a) the one or more antigen binding domains, b) optionally a linker and c) a dimerization domain or,
b. two polypeptide chains and wherein each polypeptide chain comprises in a N- to C-terminal fashion an amino acid sequence of formula I: 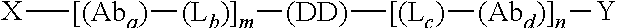
wherein m is 0, 1, 2 or an integer greater than 2;
wherein n is 0, 1, 2 or an integer greater than 2;
wherein m and n are not 0 simultaneously;
wherein Abₐ, Ab_{d}, each represents an antigen binding domain wherein at least one of Abₐ, or Ab_{d} is the one or more antigen binding domains;
wherein X or Y are independently present or absent and comprises an amino acid sequence;
wherein L_{b}, L_{c}, each independently comprises one or more linkers; and
wherein DD represents a dimerization domain, optionally wherein the dimerization domain comprises a CH3 domain of an antibody or a portion thereof and optionally a CH2 domain of an antibody or a portion thereof.

34.-36. (canceled)

37. The binding agent of claim 33, wherein the CH3 domain and/or CH2 domain is from a human IgG1, human IgG2, human IgG3 or human IgG4 heavy chain, or wherein the dimerization domain comprises an amino acid sequence at least 80% identical to the amino acid sequence set forth in SEQ ID NO:212 or SEQ ID NO:506.

38. (canceled)

39. (canceled)

40. The binding agent of claim 1, wherein the binding agent comprises a Fc or Fc portion that is capable of binding to an Fc receptor of monocytes and/or macrophages.

41. (canceled)

42. (canceled)

43. The binding agent of claim 1, wherein the binding agent is a heavy chain only antibody or comprises a heavy chain only antibody.

44. The binding agent of claim 1, wherein the binding agent is naked, or wherein the binding agent is conjugated with a therapeutic moiety and/or with a detectable moiety.

45. (canceled)

46. (canceled)

47. The binding agent of claim 1, wherein the one or more antigen binding domains or the binding agent specifically binds to human TROP2.

48. The binding agent of claim 1, wherein the binding agent is a heavy chain only antibody comprising two identical heavy chain variable regions or two identical heavy chains.

49. A pharmaceutical composition comprising the binding agent of claim 1 and a pharmaceutically acceptable carrier.

50. A nucleic acid or set of nucleic acids encoding the binding agent of claim 1.

51. A vector comprising the nucleic acid of claim 50.

52. A cell expressing the binding agent of claim 1.

53.-55. (canceled)

56. A method of treating a disorder or disease associated with TROP2 expression or overexpression comprising administering the binding agent of claim 1 or a pharmaceutical composition comprising the binding agent.

57. (canceled)

58. The method of claim 56, wherein the disorder or disease is cancer, solid tumor, epithelial cancer, lung cancer, metastatic lung cancer, small cell lung cancer, non-small cell lung cancer, myeloma, prostate cancer, breast cancer, triple negative breast cancer, rectal cancer, pancreatic cancer, glioblastoma, cervical cancer, colorectal cancer, gastric cancer, ovarian cancer, thyroid cancer, stomach cancer, urinary bladder cancer, cancer of the uterus, esophageal cancer, blood cancer, or head and neck cancer.
